(19) United States
(12) Patent Application Publication
(10) Pub. No.: US 2019/0372023 A1
(43) Pub. Date: Dec. 5, 2019

(54) ORGANIC ELECTROLUMINESCENT ELEMENT

(71) Applicants: KWANSEI GAKUIN EDUCATIONAL FOUNDATION, Nishinomiya-shi, Hyogo (JP); JNC CORPORATION, Tokyo (JP)

(72) Inventors: Takuji HATAKEYAMA, Sanda-shi (JP); Yukihiro FUJITA, Ichihara-shi (JP); Kazushi SHIREN, Ichihara-shi (JP)

(73) Assignees: Kwansei Gakuin Education Foundation, Nishinomiya-shi, Hyogo (JP); JNC Corporation, Tokyo (JP)

(21) Appl. No.: 16/477,943

(22) PCT Filed: Dec. 25, 2017
(86) PCT No.: PCT/JP2017/046373

§ 371 Date: Jul. 15, 2019

(30) Foreign Application Priority Data
Feb. 9, 2017 (JP) 2017-021790

(51) Int. Cl. H01L 51/00

(52) U.S. Cl. CPC H01L 51/0073; H01L 51/0072; H01L 51/0074; C07D 209/82; H01L 51/0056; H01L 51/008; H01L 51/0061; H01L 51/0035

(57) ABSTRACT

An object of the present invention is to provide an organic electroluminescent element having optimal light emitting characteristics. The above problem is solved by an organic electroluminescent element having a light emitting layer containing a compound of formula (1) or a multimer compound having a plurality of structures of formula (1) and a compound of formula (2).

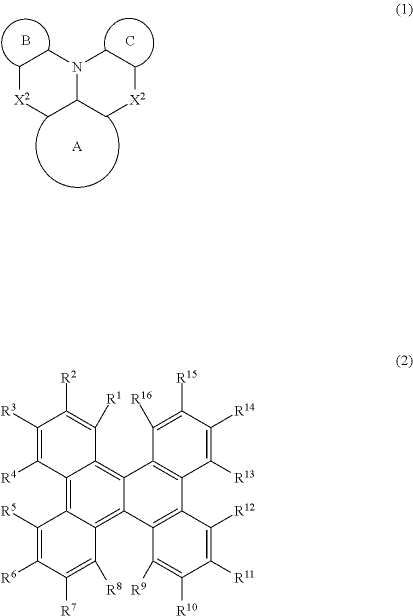

(In formula (1), ring A, ring B, and ring C each represent an aryl ring or the like, X¹ and X² each independently represent O or N—R, and the R represents an aryl or the like. In formula (2), R¹ to R¹⁶ each represent an aryl or the like.)

### TECHNICAL FIELD

The present invention relates to an organic electroluminescent element having a light emitting layer containing a specific compound as a dopant material and a specific compound as a host material, and a display apparatus and a lighting apparatus using the same.

### BACKGROUND ART

Conventionally, a display apparatus employing a luminescent element that is electroluminescent can be subjected to reduction of power consumption and thickness reduction, and therefore various studies have been conducted thereon. Furthermore, an organic electroluminescent element (hereinafter, referred to as an organic EL element) formed from an organic material has been studied actively because weight reduction or size expansion can be easily achieved. Particularly, active studies have been hitherto conducted on development of an organic material having light emitting characteristics for blue light which is one of the primary colors of light, or the like, and a combination of a plurality of materials having optimum light emitting characteristics, irrespective of whether the organic material is a high molecular weight compound or a low molecular weight compound.

An organic EL element has a structure having a pair of electrodes composed of a positive electrode and a negative electrode, and a single layer or a plurality of layers disposed between the pair of electrodes and containing an organic compound. The layer containing an organic compound includes a light emitting layer and a charge transport/injection layer for transporting or injecting charges such as holes or electrons. Various organic materials suitable for these layers have been developed.

As a material for a light emitting layer, for example, a benzofluorene-based compound has been developed (WO 2004/061047 A). Furthermore, as a hole transport material, for example, a triphenylamine-based compound has been developed (JP 2001-172232 A). Furthermore, as an electron transport material, for example, an anthracene-based compound has been developed (JP 2005-170911 A).

Furthermore, in recent years, a compound having a plurality of aromatic rings fused with a boron atom or the like as a central atom has also been reported (WO 2015/102118 A). This literature has evaluated an organic EL element in a case where the compound having a plurality of aromatic rings fused is selected as a dopant material of a light emitting layer, and particularly an anthracene-based compound (BH1 on page 442) or the like is selected among a very large number of materials described as a host material. However, a combination other than the above combination has not been specifically verified. Furthermore, if a combination constituting the light emitting layer is different, light emitting characteristics are also different. Therefore, characteristics obtained from another combination have not been found.

### CITATION LIST

### Patent Literature

Patent Literature 1: WO 2004/061047 A

Patent Literature 2: JP 2001-172232 A

Patent Literature 3: JP 2005-170911 A

Patent Literature 4: WO 2015/102118 A

### SUMMARY OF INVENTION

### Technical Problem

As described above, various materials used in an organic EL element have been developed. However, in order to further enhance light emitting characteristics or to increase options of a material for a light emitting layer, it is desired to develop a combination of materials different from a conventional combination. Particularly, organic EL characteristics (particularly optimal light emitting characteristics) obtained from a combination other than the specific combination of host and dopant reported in Examples of WO 2015/102118 A have not been found.

### Solution to Problem

As a result of intensive studies to solve the above problems, the present inventors have found that an excellent organic EL element can be obtained by disposing a light emitting layer containing a polycyclic aromatic compound having a plurality of aromatic rings linked with a boron atom and a nitrogen atom or an oxygen atom and a specific compound between a pair of electrodes to constitute an organic EL element, and have completed the present invention.

Item 1. An organic electroluminescent element comprising a pair of electrodes composed of a positive electrode and a negative electrode and a light emitting layer disposed between the pair of electrodes, in which

the light emitting layer comprises at least one selected from the group consisting of a compound represented by the following general formula (1) and a multimer having a plurality of structures represented by the following general formula (1), and a compound represented by the following general formula (2).

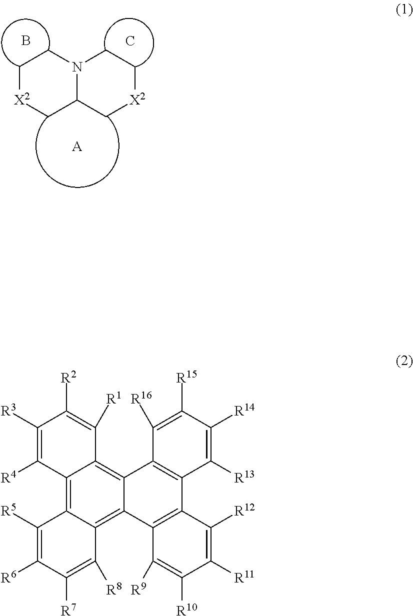

(In the above formula (1),

ring A, ring B and ring C each independently represent an aryl ring or a heteroaryl ring, while at least one hydrogen atom in these rings may be substituted,

X¹ and X² each independently represent O or N—R, R of the N—R is an optionally substituted aryl, an optionally substituted heteroaryl or an optionally substituted alkyl, R of the N—R may be bonded to the ring A, ring B, and/or ring C with a linking group or a single bond, and

at least one hydrogen atom in a compound or a structure represented by formula (1) may be substituted by a halogen atom, a cyano or a deuterium atom.)

(In the above formula (2),

R¹ to R¹⁶ each independently represent a hydrogen atom, an aryl, a heteroaryl (the heteroaryl may be bonded to the dibenzochrysene skeleton in the above formula (2) via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl,

adjacent groups among R¹ to R¹⁶ may be bonded to each other to form a fused ring, and at least one hydrogen atom in the formed ring may be substituted by an aryl, a heteroaryl (the heteroaryl may be bonded to the formed ring via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, and

at least one hydrogen atom in the compound represented by formula (2) may be substituted by a halogen atom, a cyano, or a deuterium atom.)

Item 2. The organic electroluminescent element described in item 1, in which

in the above formula (2),

R¹, R⁴, R⁵, R⁸, R⁹, R¹², R¹³, and R¹⁶ each represent a hydrogen atom,

R², R³, R⁶, R⁷, R¹⁰, R¹¹, R¹⁴, and R¹⁵ each independently represent a hydrogen atom, an aryl, a heteroaryl (the heteroaryl may be bonded to the dibenzochrysene skeleton in the above formula (2) via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, and

at least one hydrogen atom in the compound represented by the above formula (2) may be substituted by a halogen atom, a cyano, or a deuterium atom.

Item 3. The organic electroluminescent element described in item 1, in which

in the above formula (2),

R¹, R⁴, R⁵, R⁸, R⁹, R¹², R¹³, and R¹⁶ each represent a hydrogen atom, R², R³, R⁶, R⁷, R¹⁰, R¹¹, R¹⁴, and R¹⁵ each independently represent a hydrogen atom, an aryl having 6 to 30 carbon atoms, a heteroaryl having 2 to 30 carbon atoms (the heteroaryl may be bonded to the dibenzochrysene skeleton in the above formula (2) via a linking group), a diarylamino having 8 to 30 carbon atoms, a diheteroarylamino having 4 to 30 carbon atoms, an arylheteroarylamino having 4 to 30 carbon atoms, an alkyl having 1 to 30 carbon atoms, an alkenyl having 1 to 30 carbon atoms, an alkoxy having 1 to 30 carbon atoms, or an aryloxy having 1 to 30 carbon atoms, while at least one hydrogen atom in these may be substituted by an aryl having 6 to 14 carbon atoms, a heteroaryl having 2 to 20 carbon atoms, or an alkyl having 1 to 12 carbon atoms, and

at least one hydrogen atom in the compound represented by the above formula (2) may be substituted by a halogen atom, a cyano, or a deuterium atom.

Item 4. The organic electroluminescent element described in item 1, in which

in the above formula (2),

R¹, R⁴, R⁵, R⁸, R⁹, R¹², R¹³, and R¹⁶ each represent a hydrogen atom,

R², R³, R⁶, R⁷, R¹⁰, R¹¹, R¹⁴, and R¹⁵ each represent a hydrogen atom, a phenyl, a biphenylyl, a naphthyl, an anthracenyl, a phenanthrenyl, a monovalent group having a structure of the following formula (2-Ar1), (2-Ar2), (2-Ar3), (2-Ar4), or (2-Ar5) (the monovalent group having the structure may be bonded to the dibenzochrysene skeleton in the above formula (2) via a phenylene, a biphenylene, a naphthylene, an anthracenylene, a methylene, an ethylene, —OCH₂CH₂—, —CH₂CH₂O—, or —OCH₂CH₂O—), a methyl, an ethyl, a propyl, or a butyl, while at least one hydrogen atom in these may be substituted by a phenyl, a biphenylyl, a naphthyl, an anthracenyl, a phenanthrenyl, a monovalent group having a structure of the following formula (2-Ar1), (2-Ar2), (2-Ar3), (2-Ar4), or (2-Ar5), a methyl, an ethyl, a propyl, or a butyl, and

at least one hydrogen atom in the compound represented by the above formula (2) may be substituted by a halogen atom, a cyano, or a deuterium atom.

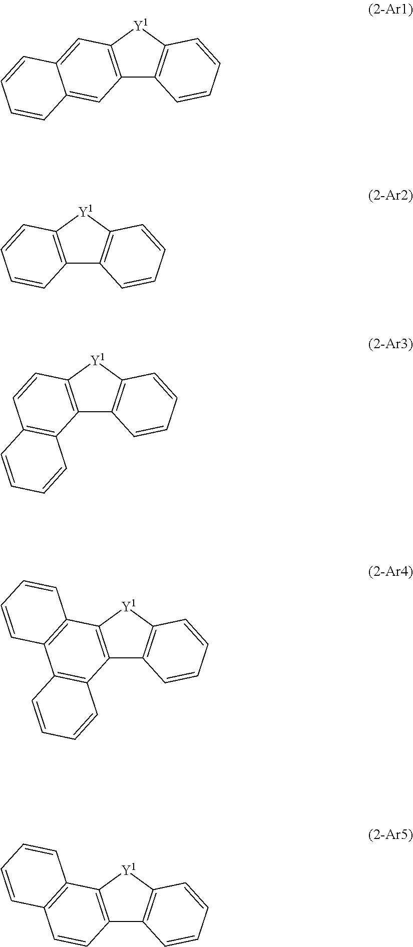

(In the above formulas (2-Ar1) to (2-Ar5), Y¹'s each independently represent O, S, or N—R, and R represents a phenyl, a biphenylyl, a naphthyl, an anthracenyl, or a hydrogen atom,

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl, and

at least one hydrogen atom in the structures represented by the above formulas (2-Ar1) to (2-Ar5) may be bonded to any one of R¹ to R¹⁶ in the above formula (2) to form a single bond.)

Item 5. The organic electroluminescent element described in item 1, in which

in the above formula (2),

R¹, R², R⁴, R⁵, R⁷, R⁸, R⁹, R¹⁰, R¹², R¹³, R¹⁵, and R¹⁶ each represent a hydrogen atom,

at least one of R³, R⁶, R¹¹, and R¹⁴ represents a monovalent group having a structure of the following formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,

a group other than the at least one represents a hydrogen atom, a phenyl, a biphenylyl, a naphthyl, an anthracenyl, a methyl, an ethyl, a propyl, or a butyl, while at least one hydrogen atom in these may be substituted by a phenyl, a biphenylyl, a naphthyl, an anthracenyl, a methyl, an ethyl, a propyl, or a butyl, and

at least one hydrogen atom in the compound represented by the above formula (2) may be substituted by a halogen atom, a cyano, or a deuterium atom.

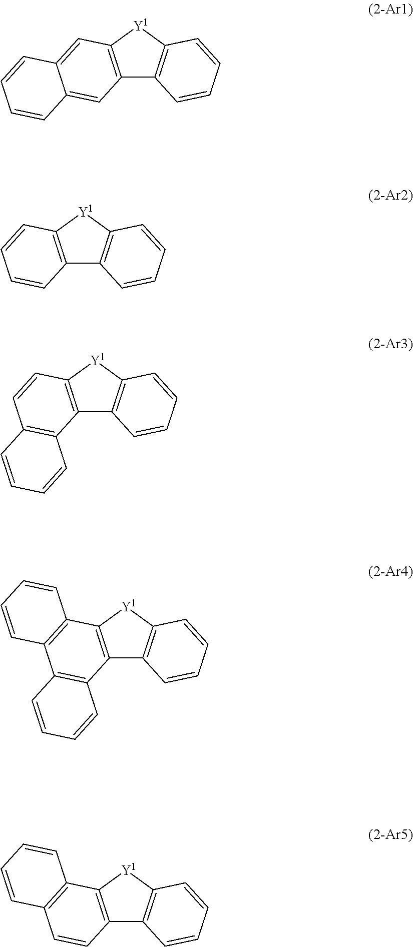

(In the formulas (2-Ar1) to (2-Ar5), Y¹'s each independently represent O, S, or N—R, and R represents a phenyl, a biphenylyl, a naphthyl, an anthracenyl, or a hydrogen atom, and

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.)

Item 6. The organic electroluminescent element described in item 5, in which

in the above formula (2),

R¹, R², R⁴, R⁵, R⁷, R⁸, R⁹, R¹⁰, R¹², R¹³, R¹⁵, and R¹⁶ each represent a hydrogen atom,

at least one of R³, R⁶, R¹¹, and R¹⁴ represents a monovalent group having a structure of the above formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,

a group other than the at least one represents a hydrogen atom, a phenyl, a biphenylyl, a naphthyl, an anthracenyl, a methyl, an ethyl, a propyl, or a butyl,

at least one hydrogen atom in the compound represented by the above formula (2) may be substituted by a halogen atom, a cyano, or a deuterium atom,

in the above formulas (2-Ar1) to (2-Ar5), Y¹'s each independently represent O, S, or N—R, and R represents a phenyl, a biphenylyl, a naphthyl, an anthracenyl, or a hydrogen atom, and

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.

Item 7. The organic electroluminescent element described in item 1, wherein the compound represented by the above formula (2) is a compound represented by any of the following structural formulas.

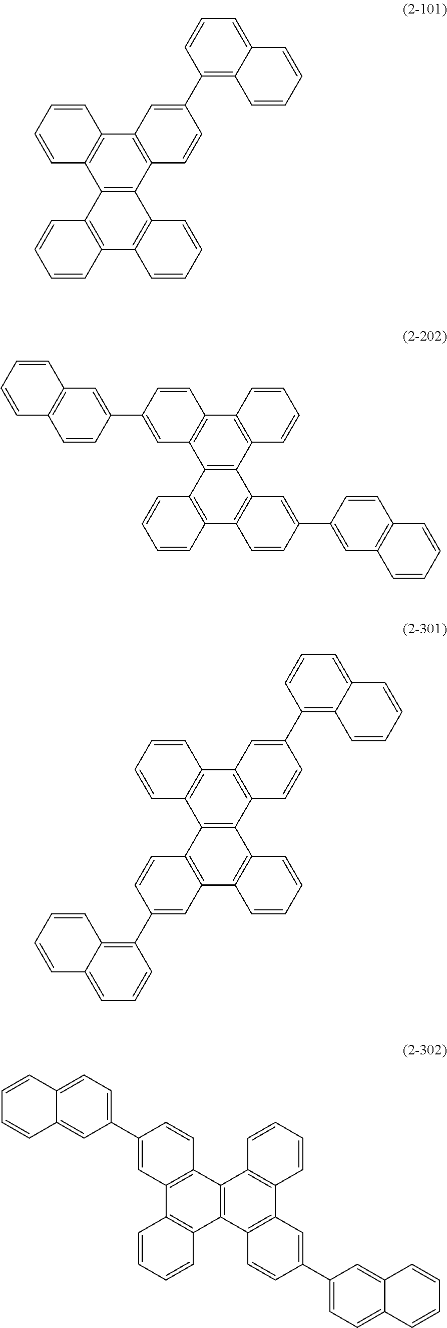

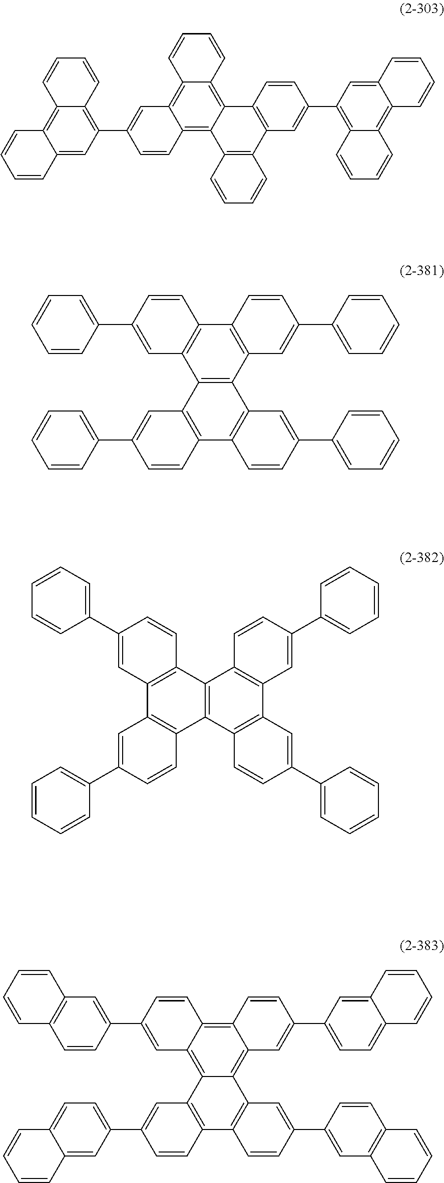

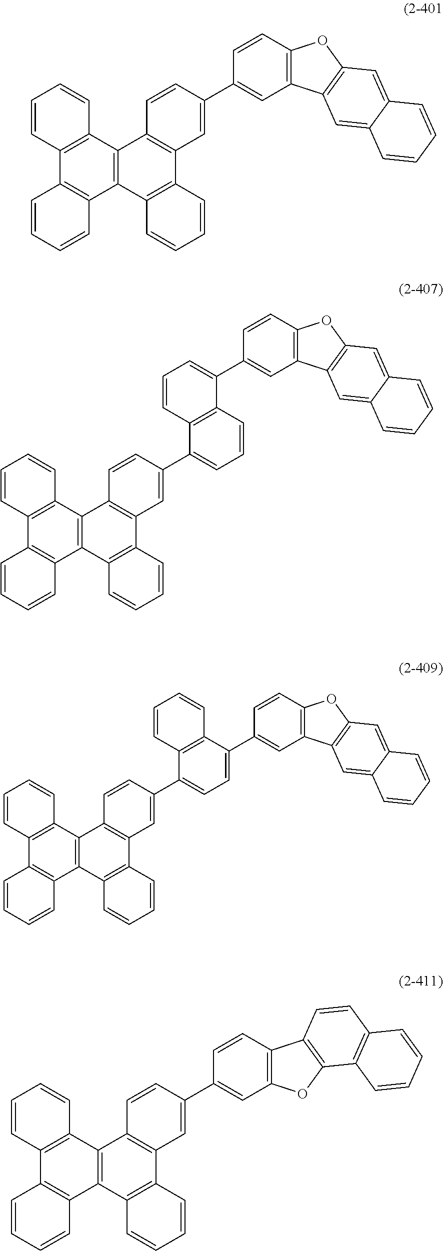 [SMILES: c1ccc2cc3c(cc2c1)oc1ccc(-c2ccc4c(c2)c2ccccc2c2c5ccccc5c5ccccc5c42)cc13 ; c1ccc2cc3c(cc2c1)oc1ccc(-c2cccc4c(-c5ccc6c(c5)c5ccccc5c5c7ccccc7c7ccccc7c65)cccc24)cc13 ; c1ccc2cc3c(cc2c1)oc1ccc(-c2ccc(-c4ccc5c(c4)c4ccccc4c4c6ccccc6c6ccccc6c54)c4ccccc24)cc13 ; c1ccc2c(c1)ccc1c3ccc(-c4ccc5c(c4)c4ccccc4c4c6ccccc6c6ccccc6c54)cc3oc21]

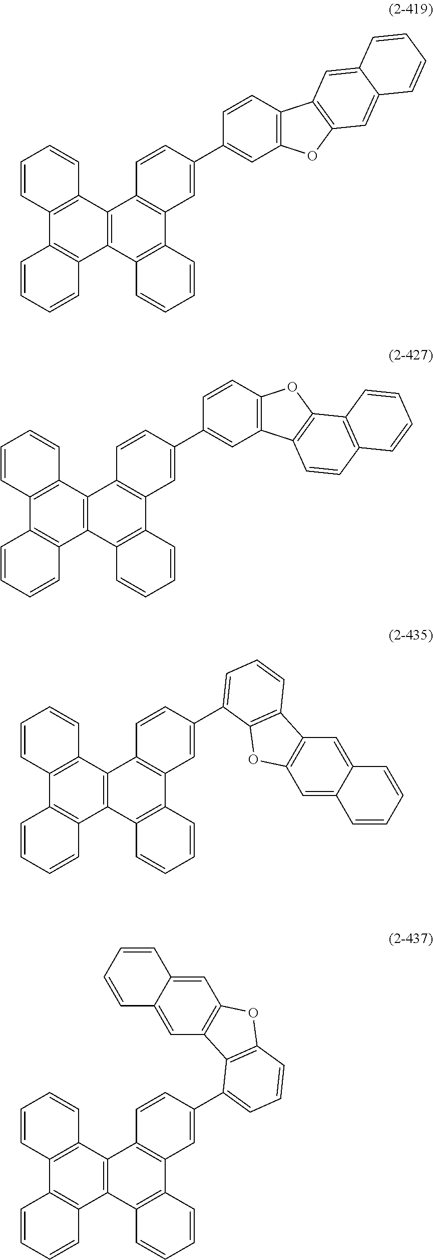 [SMILES: c1ccc2cc3c(cc2c1)oc1cc(-c2ccc4c(c2)c2ccccc2c2c5ccccc5c5ccccc5c42)ccc13 ; c1ccc2c(c1)ccc1c3cc(-c4ccc5c(c4)c4ccccc4c4c6ccccc6c6ccccc6c54)ccc3oc21 ; c1ccc2cc3c(cc2c1)oc1c(-c2ccc4c(c2)c2ccccc2c2c5ccccc5c5ccccc5c42)cccc13 ; c1ccc2cc3c(cc2c1)oc1cccc(-c2ccc4c(c2)c2ccccc2c2c5ccccc5c5ccccc5c42)c13]

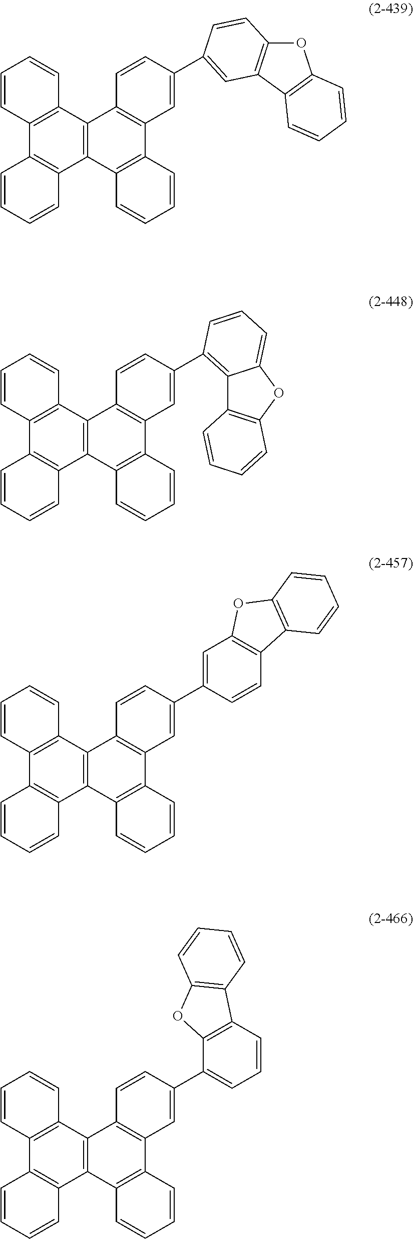 [SMILES: c1ccc2c(c1)oc1ccc(-c3ccc4c(c3)c3ccccc3c3c5ccccc5c5ccccc5c43)cc12 ; c1ccc2c(c1)oc1cccc(-c3ccc4c(c3)c3ccccc3c3c5ccccc5c5ccccc5c43)c12 ; c1ccc2c(c1)oc1cc(-c3ccc4c(c3)c3ccccc3c3c5ccccc5c5ccccc5c43)ccc12 ; c1ccc2c(c1)oc1c(-c3ccc4c(c3)c3ccccc3c3c5ccccc5c5ccccc5c43)cccc12]

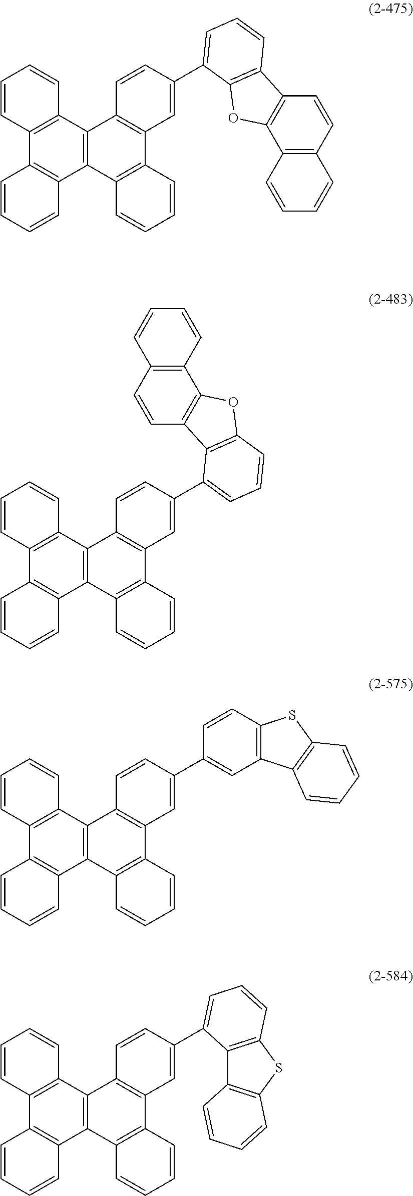 [SMILES: c1ccc2c(c1)ccc1c3cccc(-c4ccc5c(c4)c4ccccc4c4c6ccccc6c6ccccc6c54)c3oc21 ; c1ccc2c(c1)ccc1c2oc2cccc(-c3ccc4c(c3)c3ccccc3c3c5ccccc5c5ccccc5c43)c21 ; c1ccc2c(c1)sc1ccc(-c3ccc4c(c3)c3ccccc3c3c5ccccc5c5ccccc5c43)cc12 ; c1ccc2c(c1)sc1cccc(-c3ccc4c(c3)c3ccccc3c3c5ccccc5c5ccccc5c43)c12]

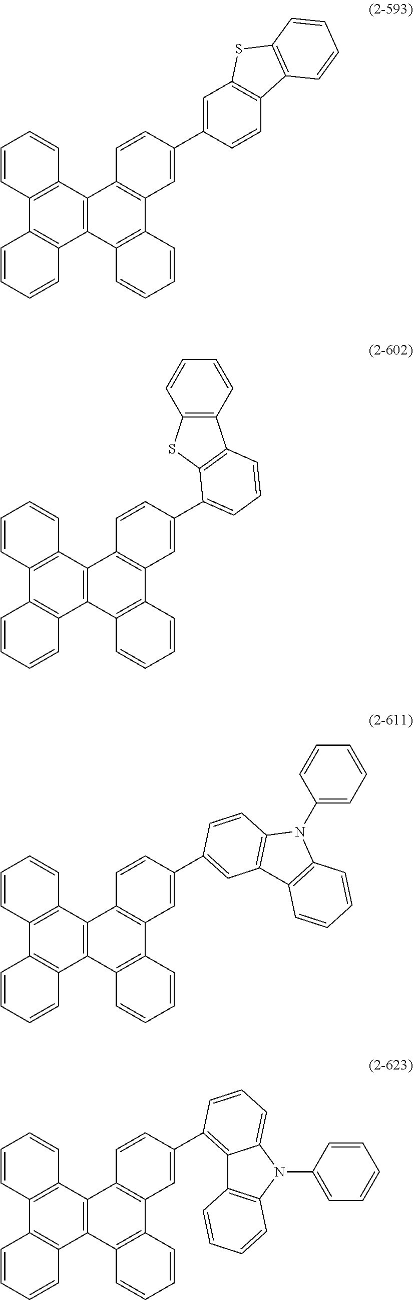 [SMILES: c1ccc2c(c1)sc1cc(-c3ccc4c(c3)c3ccccc3c3c5ccccc5c5ccccc5c43)ccc12 ; c1ccc2c(c1)sc1c(-c3ccc4c(c3)c3ccccc3c3c5ccccc5c5ccccc5c43)cccc12 ; c1ccc(-n2c3ccccc3c3cc(-c4ccc5c(c4)c4ccccc4c4c6ccccc6c6ccccc6c54)ccc32)cc1 ; c1ccc(-n2c3ccccc3c3c(-c4ccc5c(c4)c4ccccc4c4c6ccccc6c6ccccc6c54)cccc32)cc1]

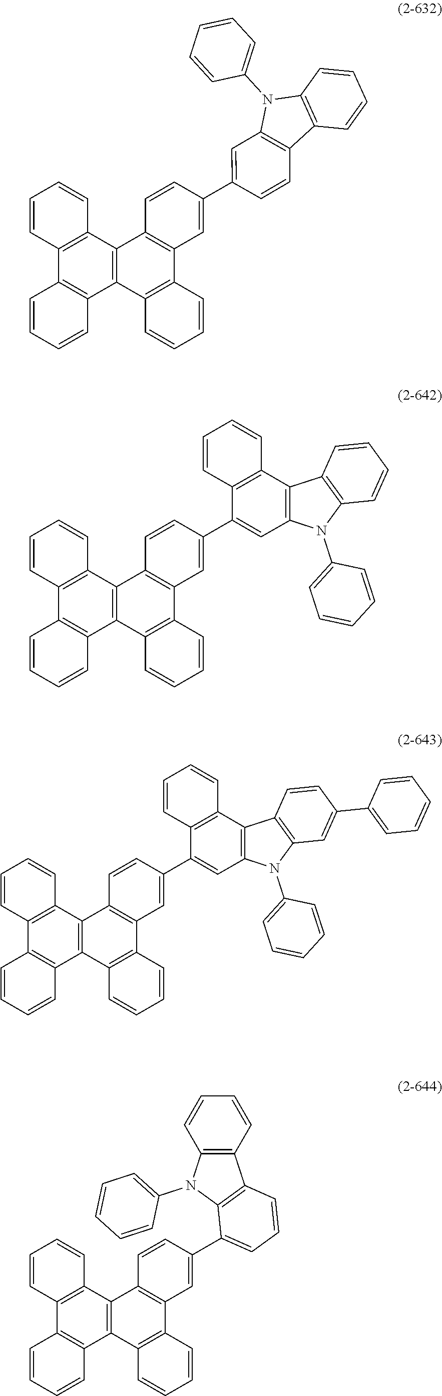 [SMILES: c1ccc(-n2c3ccccc3c3ccc(-c4ccc5c(c4)c4ccccc4c4c6ccccc6c6ccccc6c54)cc32)cc1 ; c1ccc(-n2c3ccccc3c3c4ccccc4c(-c4ccc5c(c4)c4ccccc4c4c6ccccc6c6ccccc6c54)cc32)cc1 ; c1ccc(-c2ccc3c4c5ccccc5c(-c5ccc6c(c5)c5ccccc5c5c7ccccc7c7ccccc7c65)cc4n(-c4ccccc4)c3c2)cc1 ; c1ccc(-n2c3ccccc3c3cccc(-c4ccc5c(c4)c4ccccc4c4c6ccccc6c6ccccc6c54)c32)cc1]

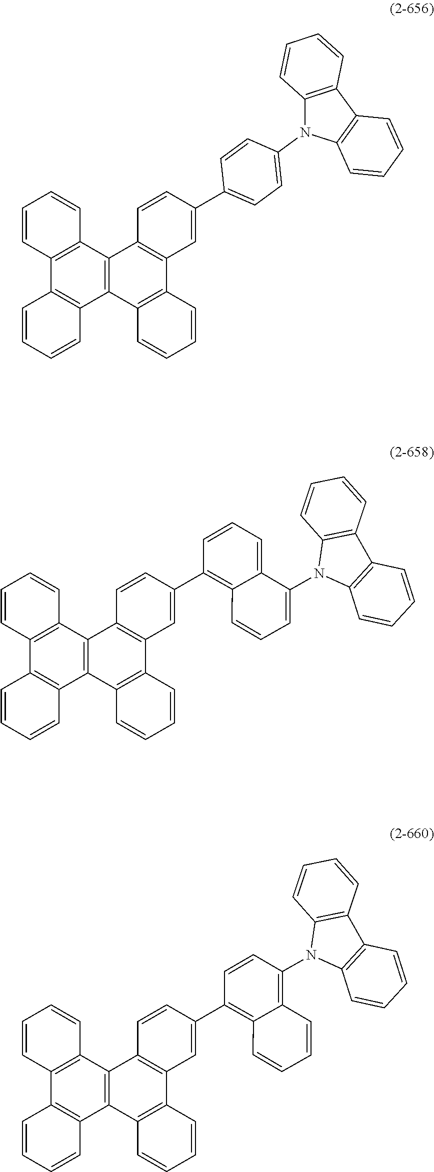 [SMILES: c1ccc2c(c1)c1ccccc1c1c3ccc(-c4ccc(-n5c6ccccc6c6ccccc65)cc4)cc3c3ccccc3c21 ; c1cc(-n2c3ccccc3c3ccccc32)c2cccc(-c3ccc4c(c3)c3ccccc3c3c5ccccc5c5ccccc5c43)c2c1 ; c1ccc2c(-n3c4ccccc4c4ccccc43)ccc(-c3ccc4c(c3)c3ccccc3c3c5ccccc5c5ccccc5c43)c2c1]

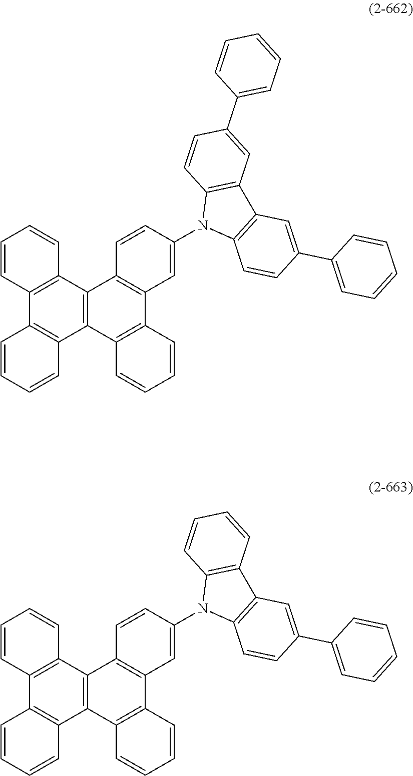 [SMILES: c1ccc(-c2ccc3c(c2)c2cc(-c4ccccc4)ccc2n3-c2ccc3c(c2)c2ccccc2c2c4ccccc4c4ccccc4c32)cc1 ; c1ccc(-c2ccc3c(c2)c2ccccc2n3-c2ccc3c(c2)c2ccccc2c2c4ccccc4c4ccccc4c32)cc1]

Item 8. The organic electroluminescent element described in any one of items 1 to 7, in which

in the above formula (1), the ring A, ring B, and ring C each independently represent an aryl ring or a heteroaryl ring, while at least one hydrogen atom in these rings may be substituted by a substituted or unsubstituted aryl, a substituted or unsubstituted heteroaryl, a substituted or unsubstituted diarylamino, a substituted or unsubstituted diheteroarylamino, a substituted or unsubstituted arylheteroarylamino, a substituted or unsubstituted alkyl, a substituted or unsubstituted alkoxy, or a substituted or unsubstituted aryloxy, each of these rings has a 5-membered or 6-membered ring sharing a bond with a fused bicyclic structure at the center of the above formula constructed by B, X¹, and X²,

X¹ and X² each independently represent O or N—R, R of the N—R each independently represents an aryl which may be substituted by an alkyl, a heteroaryl which may be substituted by an alkyl or alkyl, R of the N—R may be bonded to the ring A, ring B, and/or ring C with —O—, —S—, —C(—R)₂— or a single bond, R of the —C(—R)₂— represents a hydrogen atom or an alkyl,

at least one hydrogen atom in a compound or structure represented by formula (1) may be substituted by a halogen atom, a cyano or a deuterium atom, and in a case of a multimer, the multimer is a dimer or a trimer having two or three structures represented by formula (1).

Item 9. The organic electroluminescent element described in any one of items 1 to 8, wherein the compound represented by the above general formula (1) is a compound represented by the following general formula (1′).

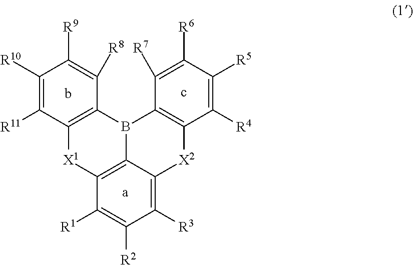

(In the above formula (1′),

R¹ to R¹¹ each independently represent a hydrogen atom, an aryl, a heteroaryl, a diarylamino, a diheteroarylamino, an arylheteroarylamino, an alkyl, an alkoxy, or an aryloxy, while at least one hydrogen atom in these may be substituted by an aryl, a heteroaryl, or an alkyl, adjacent groups among R¹ to R¹¹ may be bonded to each other to form an aryl ring or a heteroaryl ring together with ring a, ring b, or ring c, at least one hydrogen atom in the ring thus formed may be substituted by an aryl, a heteroaryl, a diarylamino, a diheteroarylamino, an arylheteroarylamino, an alkyl, an alkoxy, or an aryloxy, at least one hydrogen atom in these may be substituted by an aryl, a heteroaryl or an alkyl,

X¹ and X² each independently represent N—R, R of the N—R represents an aryl having 6 to 12 carbon atoms, a heteroaryl having 2 to 15 carbon atoms, or an alkyl having 1 to 6 carbon atoms, R of the N—R may be bonded to the ring a, ring b and/or ring c with —O—, —S—, —C(—R)₂—, or a single bond, R of the —C(—R)₂— represents an alkyl having 1 to 6 carbon atoms, and

at least one hydrogen atom in a compound represented by formula (1′) may be substituted by a halogen atom or a deuterium atom.)

Item 10. The organic electroluminescent element described in item 9, in which

in the above formula (1′),

R¹ to R¹¹ each independently represent a hydrogen atom, an aryl having 6 to 30 carbon atoms, a heteroaryl having 2 to 30 carbon atoms or a diarylamino (the aryl is an aryl having 6 to 12 carbon atoms), adjacent groups among R¹ to R¹¹ may be bonded to each other to form an aryl ring having 9 to 16 carbon atoms or a heteroaryl ring having 6 to 15 carbon atoms together with the ring a, ring b, or ring c, at least one hydrogen atom in the ring thus formed may be substituted by an aryl having 6 to 10 carbon atoms,

X¹ and X² each independently represent N—R, R of the N—R is an aryl having 6 to 10 carbon atoms, and

at least one hydrogen atom in a compound represented by formula (1′) may be substituted by a halogen atom or a deuterium atom.

Item 11. The organic electroluminescent element described in any one of items 1 to 10, wherein the compound represented by the above formula (1) is a compound represented by any of the following structural formulas.

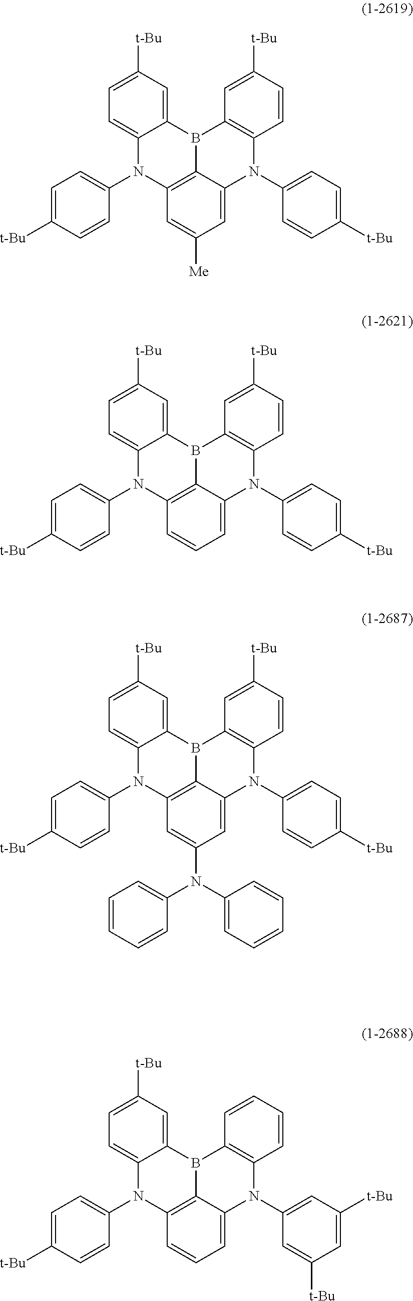 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4ccccc4N(c4cc(C(C)(C)C)cc(C(C)(C)C)c4)c4cccc2c43)cc1]

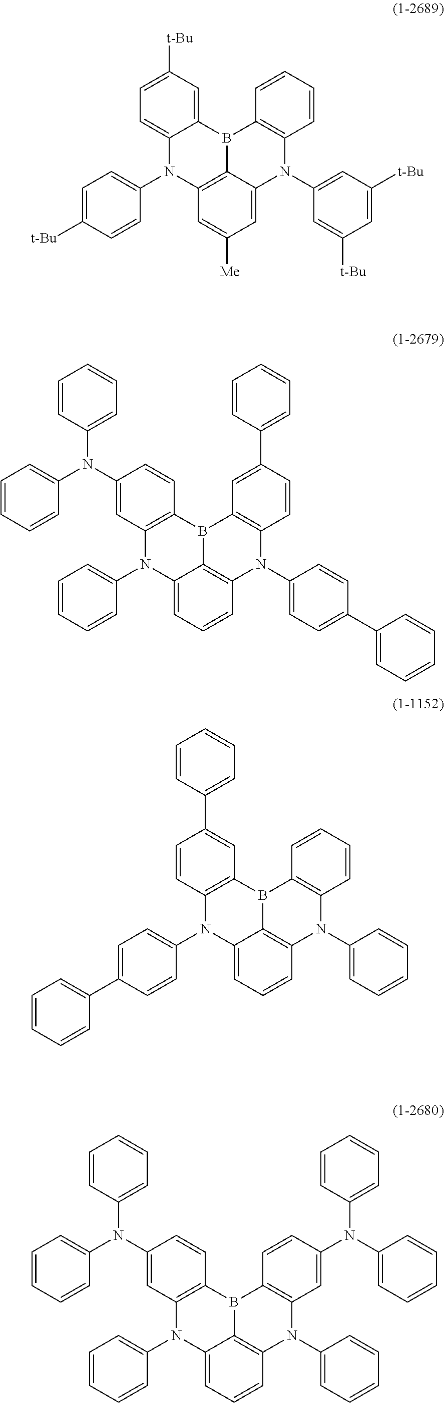 [SMILES: Cc1cc2c3c(c1)N(c1ccc(C(C)(C)C)cc1)c1ccc(C(C)(C)C)cc1B3c1ccccc1N2c1cc(C(C)(C)C)cc(C(C)(C)C)c1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

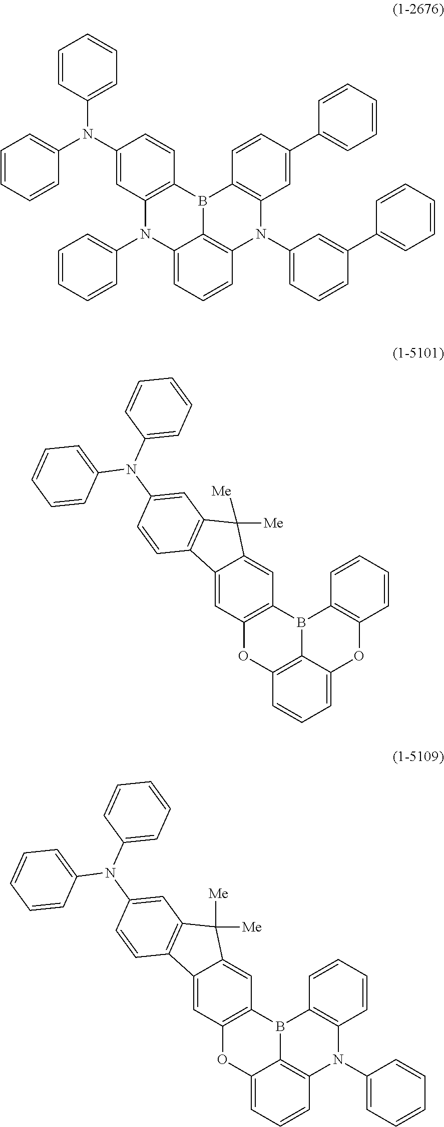 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; CC1(C)c2cc(N(c3ccccc3)c3ccccc3)ccc2-c2cc3c(cc21)B1c2ccccc2Oc2cccc(c21)O3 ; CC1(C)c2cc(N(c3ccccc3)c3ccccc3)ccc2-c2cc3c(cc21)B1c2ccccc2N(c2ccccc2)c2cccc(c21)O3]

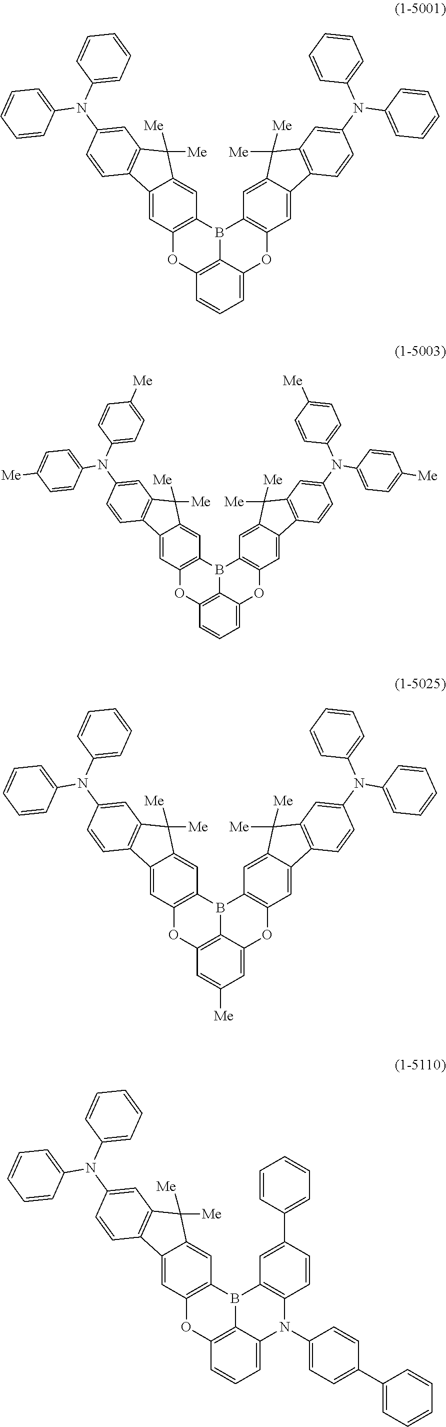 [SMILES: CC1(C)c2cc(N(c3ccccc3)c3ccccc3)ccc2-c2cc3c(cc21)B1c2cc4c(cc2Oc2cccc(c21)O3)-c1ccc(N(c2ccccc2)c2ccccc2)cc1C4(C)C ; Cc1ccc(N(c2ccc(C)cc2)c2ccc3c(c2)C(C)(C)c2cc4c(cc2-3)Oc2cccc3c2B4c2cc4c(cc2O3)-c2ccc(N(c3ccc(C)cc3)c3ccc(C)cc3)cc2C4(C)C)cc1 ; Cc1cc2c3c(c1)Oc1cc4c(cc1B3c1cc3c(cc1O2)-c1ccc(N(c2ccccc2)c2ccccc2)cc1C3(C)C)C(C)(C)c1cc(N(c2ccccc2)c2ccccc2)ccc1-4 ; CC1(C)c2cc(N(c3ccccc3)c3ccccc3)ccc2-c2cc3c(cc21)B1c2cc(-c4ccccc4)ccc2N(c2ccc(-c4ccccc4)cc2)c2cccc(c21)O3]

Item 12. The organic electroluminescent element described in any one of items 1 to 11, further comprising an electron transport layer and/or an electron injection layer disposed between the negative electrode and the light emitting layer, in which at least one of the electron transport layer and the electron injection layer comprises at least one selected from the group consisting of a borane derivative, a pyridine derivative, a fluoranthene derivative, a BO-based derivative, an anthracene derivative, a benzofluorene derivative, a phosphine oxide derivative, a pyrimidine derivative, a carbazole derivative, a triazine derivative, a benzimidazole derivative, a phenanthroline derivative, and a quinolinol-based metal complex.
Item 13. The organic electroluminescent element described in item 12, in which the electron transport layer and/or electron injection layer further comprise/comprises at least one selected from the group consisting of an alkali metal, an alkaline earth metal, a rare earth metal, an oxide of an alkali metal, a halide of an alkali metal, an oxide of an alkaline earth metal, a halide of an alkaline earth metal, an oxide of a rare earth metal, a halide of a rare earth metal, an organic complex of an alkali metal, an organic complex of an alkaline earth metal, and an organic complex of a rare earth metal.
Item 14. A display apparatus comprising the organic electroluminescent element described in any one of items 1 to 13.
Item 15. A lighting apparatus comprising the organic electroluminescent element described in any one of items 1 to 14.
Item 16. A compound represented by the following formula (2).

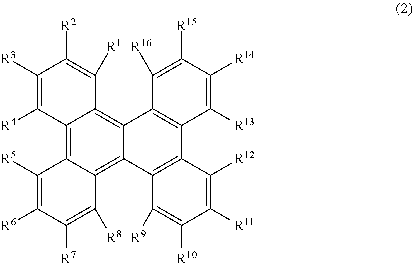

(In the above formula (2),

R¹, R², R⁴, R⁵, R⁷, R⁸, R⁹, R¹⁰, R¹², R¹³, R¹⁵, and R¹⁶ each represent a hydrogen atom,

at least one of R³, R⁶, R¹¹, and R¹⁴ represents a monovalent group having a structure of the following formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,

a group other than the at least one represents a hydrogen atom, a phenyl, a biphenylyl, a naphthyl, an anthracenyl, a methyl, an ethyl, a propyl, or a butyl, while at least one hydrogen atom in these may be substituted by a phenyl, a biphenylyl, a naphthyl, an anthracenyl, a methyl, an ethyl, a propyl, or a butyl, and

at least one hydrogen atom in the compound represented by the above formula (2) may be substituted by a halogen atom, a cyano, or a deuterium atom.

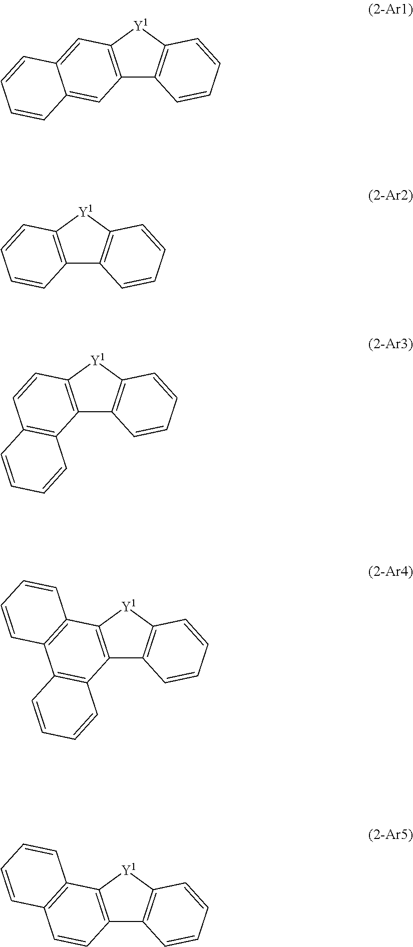

(In the formulas (2-Ar1) to (2-Ar5), Y¹'s each independently represent O, S, or N—R, and R represents a phenyl, a biphenylyl, a naphthyl, an anthracenyl, or a hydrogen atom, and

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.)

Item 17. The compound described in item 16, in which

in the above formula (2),

R¹, R², R⁴, R⁵, R⁷, R⁸, R⁹, R¹⁰, R¹², R¹³, R¹⁵, and R¹⁶ each represent a hydrogen atom,

at least one of R³, R⁶, R¹¹, and R¹⁴ represents a monovalent group having a structure of the above formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,

a group other than the at least one represents a hydrogen atom, a phenyl, a biphenylyl, a naphthyl, an anthracenyl, a methyl, an ethyl, a propyl, or a butyl,

at least one hydrogen atom in the compound represented by the above formula (2) may be substituted by a halogen atom, a cyano, or a deuterium atom,

in the above formulas (2-Ar1) to (2-Ar5), Y¹'s each independently represent O, S, or N—R, and R represents a phenyl, a biphenylyl, a naphthyl, an anthracenyl, or a hydrogen atom, and

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.

Item 18. A compound represented by any of the following structural formulas.

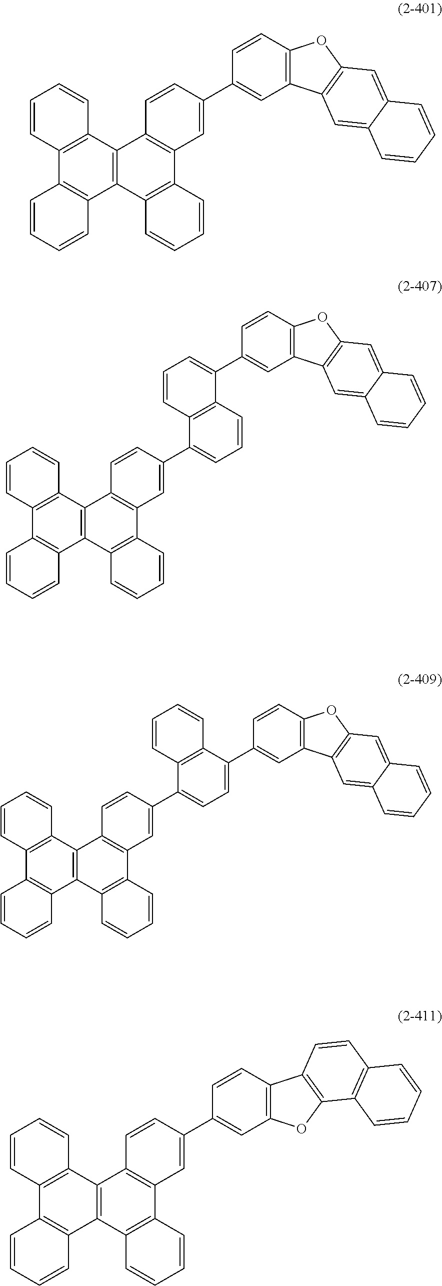 [SMILES: c1ccc2cc3c(cc2c1)oc1ccc(-c2ccc4c(c2)c2ccccc2c2c5ccccc5c5ccccc5c42)cc13 ; c1ccc2cc3c(cc2c1)oc1ccc(-c2cccc4c(-c5ccc6c(c5)c5ccccc5c5c7ccccc7c7ccccc7c65)cccc24)cc13 ; c1ccc2cc3c(cc2c1)oc1ccc(-c2ccc(-c4ccc5c(c4)c4ccccc4c4c6ccccc6c6ccccc6c54)c4ccccc24)cc13 ; c1ccc2c(c1)ccc1c3ccc(-c4ccc5c(c4)c4ccccc4c4c6ccccc6c6ccccc6c54)cc3oc21]

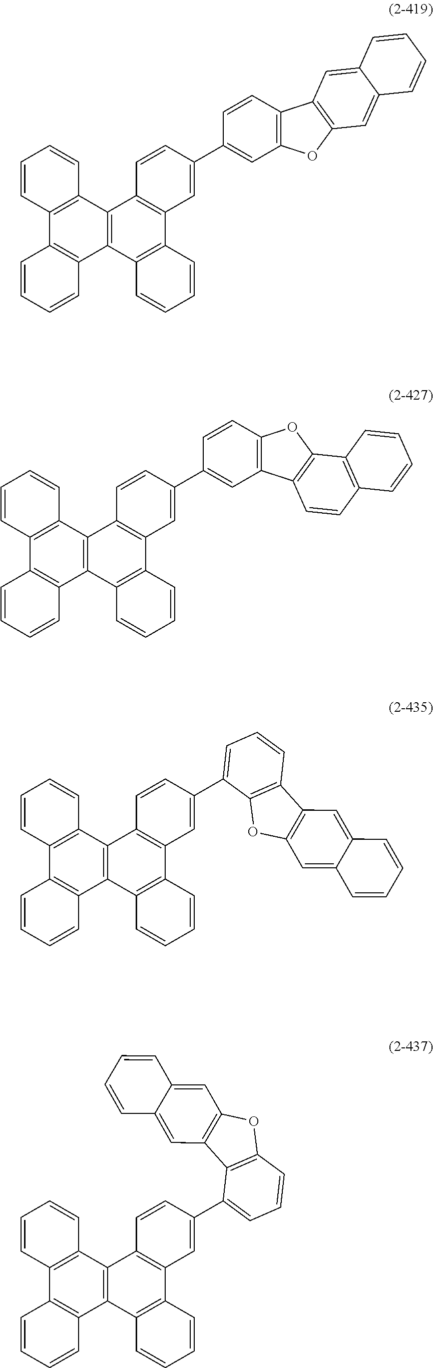 [SMILES: c1ccc2cc3c(cc2c1)oc1cc(-c2ccc4c(c2)c2ccccc2c2c5ccccc5c5ccccc5c42)ccc13 ; c1ccc2c(c1)ccc1c3cc(-c4ccc5c(c4)c4ccccc4c4c6ccccc6c6ccccc6c54)ccc3oc21 ; c1ccc2cc3c(cc2c1)oc1c(-c2ccc4c(c2)c2ccccc2c2c5ccccc5c5ccccc5c42)cccc13 ; c1ccc2cc3c(cc2c1)oc1cccc(-c2ccc4c(c2)c2ccccc2c2c5ccccc5c5ccccc5c42)c13]

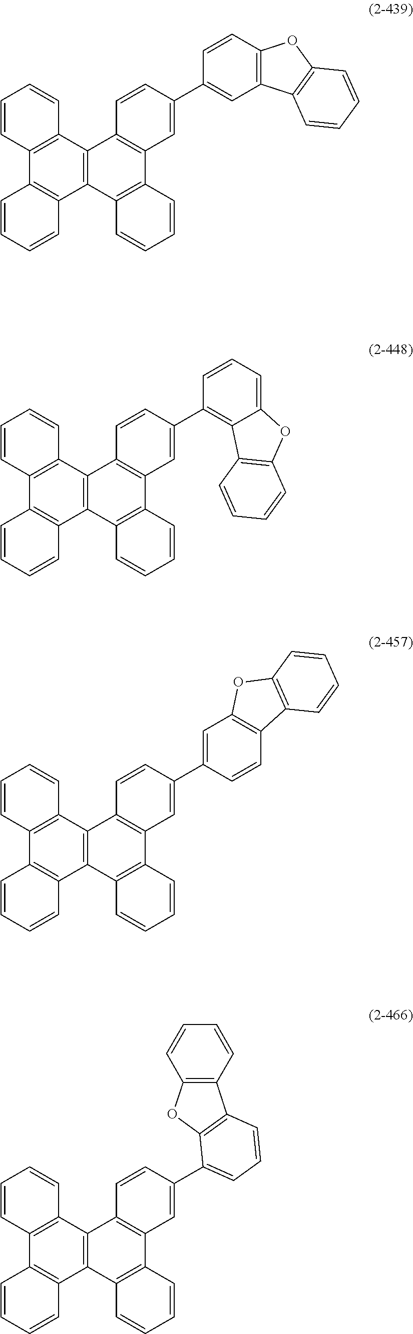 [SMILES: c1ccc2c(c1)oc1ccc(-c3ccc4c(c3)c3ccccc3c3c5ccccc5c5ccccc5c43)cc12 ; c1ccc2c(c1)oc1cccc(-c3ccc4c(c3)c3ccccc3c3c5ccccc5c5ccccc5c43)c12 ; c1ccc2c(c1)oc1cc(-c3ccc4c(c3)c3ccccc3c3c5ccccc5c5ccccc5c43)ccc12 ; c1ccc2c(c1)oc1c(-c3ccc4c(c3)c3ccccc3c3c5ccccc5c5ccccc5c43)cccc12]

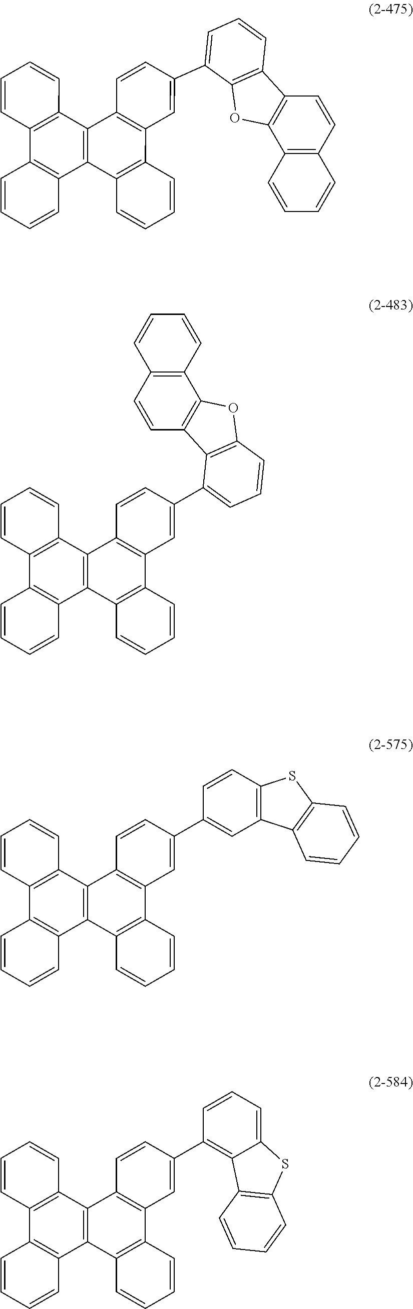 [SMILES: c1ccc2c(c1)ccc1c3cccc(-c4ccc5c(c4)c4ccccc4c4c6ccccc6c6ccccc6c54)c3oc21 ; c1ccc2c(c1)ccc1c2oc2cccc(-c3ccc4c(c3)c3ccccc3c3c5ccccc5c5ccccc5c43)c21 ; c1ccc2c(c1)sc1ccc(-c3ccc4c(c3)c3ccccc3c3c5ccccc5c5ccccc5c43)cc12 ; c1ccc2c(c1)sc1cccc(-c3ccc4c(c3)c3ccccc3c3c5ccccc5c5ccccc5c43)c12]

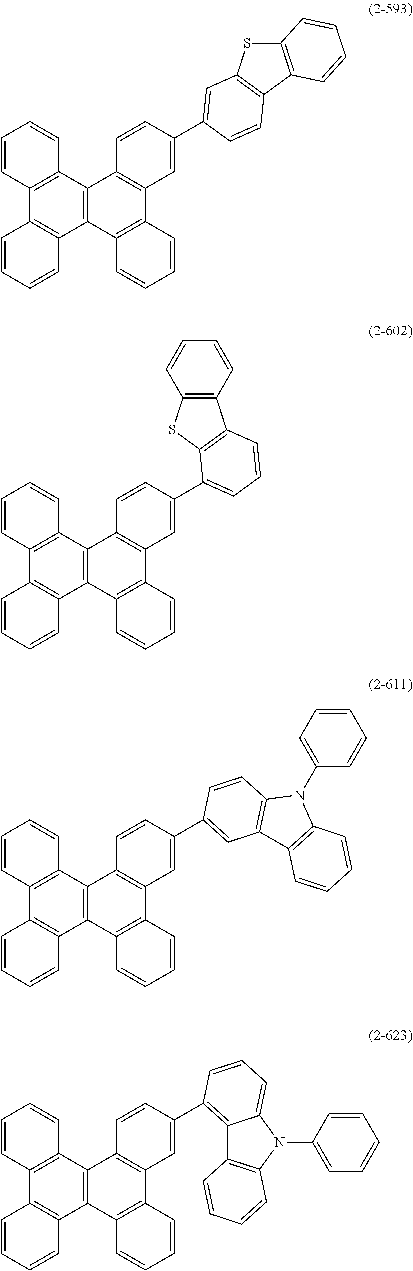 [SMILES: c1ccc2c(c1)sc1cc(-c3ccc4c(c3)c3ccccc3c3c5ccccc5c5ccccc5c43)ccc12 ; c1ccc2c(c1)sc1c(-c3ccc4c(c3)c3ccccc3c3c5ccccc5c5ccccc5c43)cccc12 ; c1ccc(-n2c3ccccc3c3cc(-c4ccc5c(c4)c4ccccc4c4c6ccccc6c6ccccc6c54)ccc32)cc1 ; c1ccc(-n2c3ccccc3c3c(-c4ccc5c(c4)c4ccccc4c4c6ccccc6c6ccccc6c54)cccc32)cc1]

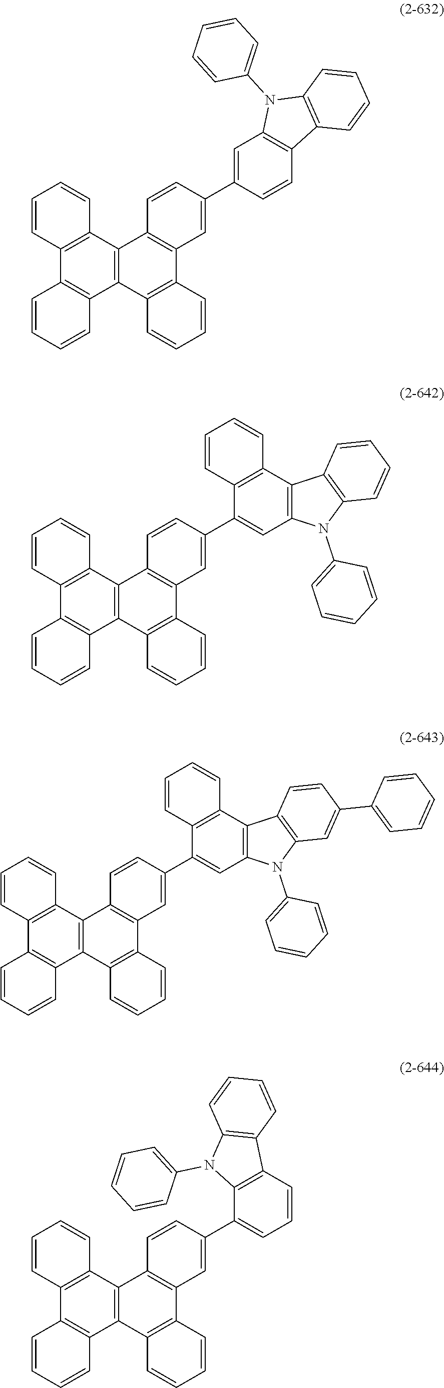 [SMILES: c1ccc(-n2c3ccccc3c3ccc(-c4ccc5c(c4)c4ccccc4c4c6ccccc6c6ccccc6c54)cc32)cc1 ; c1ccc(-n2c3ccccc3c3c4ccccc4c(-c4ccc5c(c4)c4ccccc4c4c6ccccc6c6ccccc6c54)cc32)cc1 ; c1ccc(-c2ccc3c4c5ccccc5c(-c5ccc6c(c5)c5ccccc5c5c7ccccc7c7ccccc7c65)cc4n(-c4ccccc4)c3c2)cc1 ; c1ccc(-n2c3ccccc3c3cccc(-c4ccc5c(c4)c4ccccc4c4c6ccccc6c6ccccc6c54)c32)cc1]

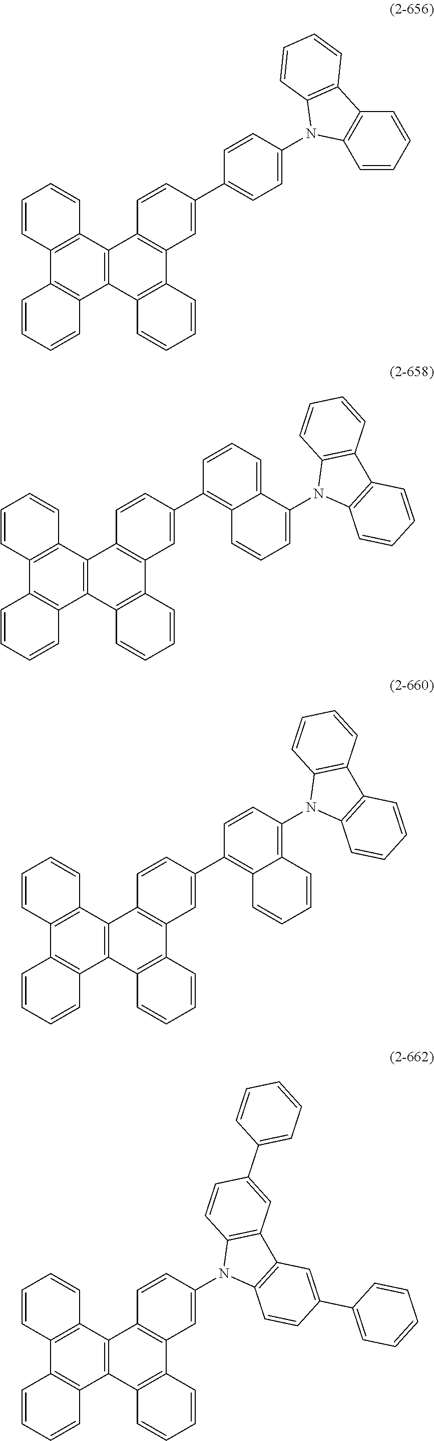 [SMILES: c1ccc2c(c1)c1ccccc1c1c3ccc(-c4ccc(-n5c6ccccc6c6ccccc65)cc4)cc3c3ccccc3c21 ; c1cc(-n2c3ccccc3c3ccccc32)c2cccc(-c3ccc4c(c3)c3ccccc3c3c5ccccc5c5ccccc5c43)c2c1 ; c1ccc2c(-n3c4ccccc4c4ccccc43)ccc(-c3ccc4c(c3)c3ccccc3c3c5ccccc5c5ccccc5c43)c2c1 ; c1ccc(-c2ccc3c(c2)c2cc(-c4ccccc4)ccc2n3-c2ccc3c(c2)c2ccccc2c2c4ccccc4c4ccccc4c32)cc1]

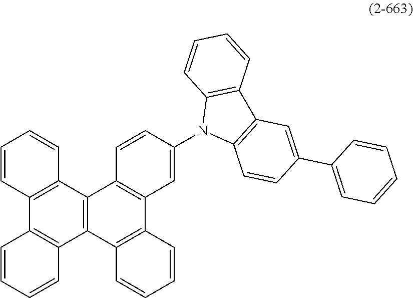 [SMILES: c1ccc(-c2ccc3c(c2)c2ccccc2n3-c2ccc3c(c2)c2ccccc2c2c4ccccc4c4ccccc4c32)cc1]

### Advantageous Effects of Invention

According to a preferable embodiment of the present invention, it is possible to provide a compound represented by formula (1) and a compound represented by formula (2), capable of obtaining optimum light emitting characteristics in combination with the compound represented by formula (1). By manufacturing an organic EL element using a material for a light emitting layer obtained by combining these compounds, it is possible to provide an organic EL element that is excellent in at least one of chromaticity, driving voltage, and quantum efficiency.

### BRIEF DESCRIPTION OF DRAWINGS

FIG. 1 is a schematic cross-sectional view illustrating an organic EL element according to the present embodiment.

### DESCRIPTION OF EMBODIMENTS

1. Characteristic Light Emitting Layer in the Organic EL Device

The present invention relates to an organic electroluminescent element including a pair of electrodes composed of a positive electrode and a negative electrode and a light emitting layer disposed between the pair of electrodes, in which the light emitting layer includes at least one selected from the group consisting of a polycyclic aromatic compound represented by the following general formula (1) and a multimer thereof having a plurality of structures each represented by the following general formula (1), and at least one compound represented by the following general formula (2).

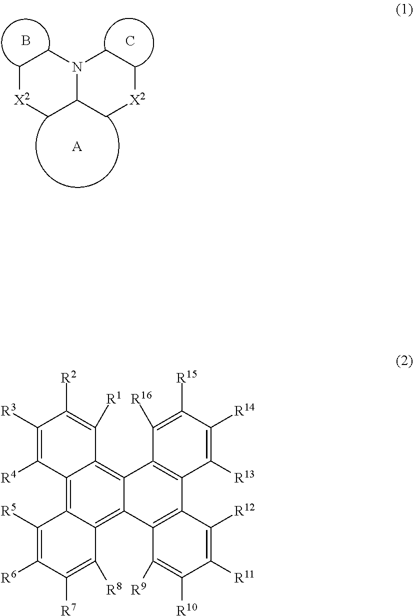

1-1. Compound Represented by Formula (1) and Multimer Thereof

Each of a compound represented by general formula (1) and a multimer having a plurality of structures represented by general formula (1) basically functions as a dopant. The compound and multimer thereof are preferably a compound represented by the following general formula (1′) or a multimer having a plurality of structures represented by the following general formula (1′). Note that in the formula (1), “B” of the central atom means a boron atom, and “B” together with “A” and “C” in the rings are a code indicating a ring structure represented by the ring.

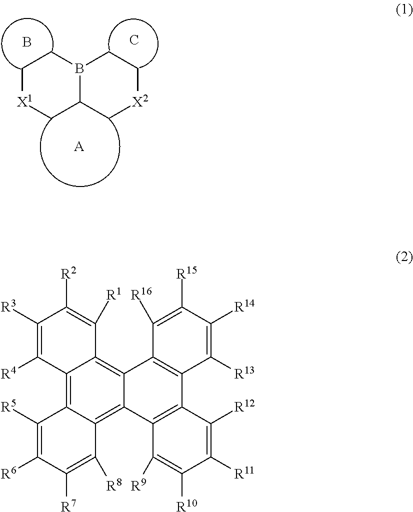

The ring A, ring B and ring C in general formula (1) each independently represent an aryl ring or a heteroaryl ring, and at least one hydrogen atom in these rings may be substituted by a substituent. This substituent is preferably a substituted or unsubstituted aryl, a substituted or unsubstituted heteroaryl, a substituted or unsubstituted diarylamino, a substituted or unsubstituted diheteroarylamino, a substituted or unsubstituted arylheteroarylamino (an amino group having an aryl and a heteroaryl), a substituted or unsubstituted alkyl, a substituted or unsubstituted alkoxy, or a substituted or unsubstituted aryloxy. In a case where these groups have substituents, examples of the substituents include an aryl, a heteroaryl, and an alkyl. Furthermore, the aryl ring or heteroaryl ring preferably has a 5-membered ring or 6-membered ring sharing a bond with a fused bicyclic structure at the center of general formula (1) constituted by “B”, “X^{1”}, and “X²” (hereinafter, this structure is also referred to as “structure D”).

Here, the “fused bicyclic structure (structure D)” means a structure in which two saturated hydrocarbon rings that are configured to include “B”, “X¹” and “X²” and indicated at the center of general formula (1) are fused. Furthermore, a “6-membered ring sharing a bond with the fused bicyclic structure” means, for example, ring a (benzene ring (6-membered ring)) fused to the structure D as represented by the above general formula (1′). Furthermore, the phrase “aryl ring or heteroaryl ring (which is ring A) has this 6-membered ring” means that the ring A is formed only from this 6-membered ring, or the ring A is formed such that other rings are further fused to this 6-membered ring so as to include this 6-membered ring. In other words, the “aryl ring or heteroaryl ring (which is ring A) having a 6-membered ring” as used herein means that the 6-membered ring that constitutes the entirety or a portion of the ring A is fused to the structure D. The same description applies to the “ring B (ring b)”, “ring C (ring c)”, and the “5-membered ring”.

The ring A (or ring B or ring C) in general formula (1) corresponds to ring a and its substituents R¹ to R³ in general formula (1′) (or ring b and its substituents R⁴ to R⁷, or ring c and its substituents R⁸ to R¹¹). That is, general formula (1′) corresponds to a structure in which “rings A to C having 6-membered rings” have been selected as the rings A to C of general formula (1). For this meaning, the rings of general formula (1′) are represented by small letters a to c.

In general formula (1′), adjacent groups among the substituents R¹ to R¹¹ of the ring a, ring b, and ring c may be bonded to each other to form an aryl ring or a heteroaryl ring together with the ring a, ring b, or ring c, and at least one hydrogen atom in the ring thus formed may be substituted by an aryl, a heteroaryl, a diarylamino, a diheteroarylamino, an arylheteroarylamino, an alkyl, an alkoxy or an aryloxy, while at least one hydrogen atom in these may be substituted by an aryl, a heteroaryl, or an alkyl. Therefore, in a compound represented by general formula (1′), a ring structure constituting the compound changes as represented by the following formulas (1′-1) and (1′-2) according to a mutual bonding form of substituents in the ring a, ring b or ring c. Ring A′, ring B′ and ring C′ in each formula correspond to the ring A, ring B and ring C in general formula (1), respectively. Note that R¹ to R¹¹, a, b, c, X¹, and X² in each formula are defined in the same manner as those in formula (1′).

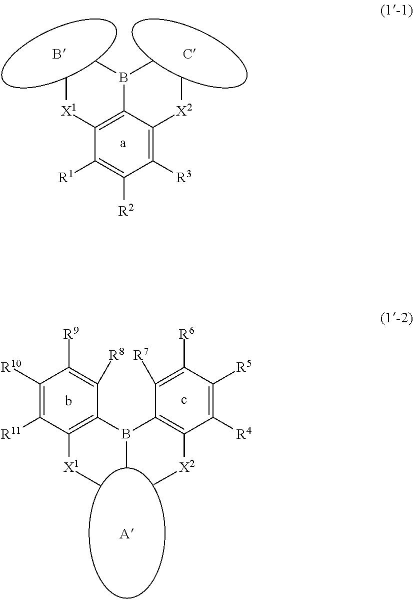

The ring A′, ring B′ and, ring C′ in the above formulas (1′-1) and (1′-2) each represent, to be described in connection with general formula (1′), an aryl ring or a heteroaryl ring formed by bonding adjacent groups among the substituents R¹ to R¹¹ together with the ring a, ring b, and ring c, respectively (may also be referred to as a fused ring obtained by fusing another ring structure to the ring a, ring b, or ring c). Incidentally, although not indicated in the formula, there is also a compound in which all of the ring a, ring b, and ring c have been changed to the ring A′, ring B′ and ring C′. Furthermore, as apparent from the above formulas (1′-1) and (1′-2), for example, R⁸ of the ring b and R⁷ of the ring c, R¹¹ of the ring b and R¹ of the ring a, R⁴ of the ring c and R³ of the ring a, and the like do not correspond to “adjacent groups”, and these groups are not bonded to each other. That is, the term “adjacent groups” means adjacent groups on the same ring.

A compound represented by the above formula (1′-1) or (1′-2) corresponds to, for example, a compound represented by any one of formulas (1-402) to (1-409) and (1-412) to (1-419) listed as specific compounds that are described below. That is, for example, the compound represented by formula (1′-1) or (1′-2) is a compound having ring A′ (or ring B′ or ring C′) that is formed by fusing a benzene ring, an indole ring, a pyrrole ring, a benzofuran ring, a benzothiophene ring or the like to a benzene ring which is ring a (or ring b or ring c), and the fused ring A′ (or fused ring B′ or fused ring C′) that has been formed is a naphthalene ring, a carbazole ring, an indole ring, a dibenzofuran ring, a dibenzothiophene ring or the like.

X¹ and X² in general formula (1) each independently represent “O” or “N—R”, while R of the N—R represents an optionally substituted aryl, or an optionally substituted heteroaryl or an optionally substituted alkyl, and R of the N—R may be bonded to the ring B and/or ring C with a linking group or a single bond. The linking group is preferably —O—, —S— or —C(—R)₂—. Incidentally, R of the “—C(—R)₂—” represents a hydrogen atom or an alkyl. This description also applies to X¹ and X² in general formula (1′).

Here, the provision that “R of the N—R is bonded to the ring A, ring B and/or ring C with a linking group or a single bond” for general formula (1) corresponds to the provision that “R of the N—R is bonded to the ring a, ring b and/or ring c with —O—, —S—, —C(—R)₂— or a single bond” for general formula (1′).

This provision can be expressed by a compound having a ring structure represented by the following formula (1′-3-1), in which X¹ or X² is incorporated into the fused ring B′ or C′. That is, for example, the compound is a compound having ring B′ (or ring C′) formed by fusing another ring to a benzene ring which is ring b (or ring c) in general formula (1′) so as to incorporate X¹ (or X²). This compound corresponds to, for example, a compound represented by any one of formulas (1-451) to (1-462) or a compound represented by any one of formulas (1-1401) to (1-1460), listed as specific examples that are described below, and the fused ring B′ (or fused ring C′) that has been formed is, for example, a phenoxazine ring, a phenothiazine ring, or an acridine ring.

The above provision can be expressed by a compound having a ring structure in which X¹ and/or X² are/is incorporated into the fused ring A′, represented by the following formula (1′-3-2) or (1′-3-3). That is, for example, the compound is a compound having ring A′ formed by fusing another ring to a benzene ring which is ring a in general formula (1′) so as to incorporate X¹ (and/or X²) This compound corresponds to, for example, a compound represented by any one of formulas (1-471) to (1-479) listed as specific examples that are described below, and the fused ring A′ that has been formed is, for example, a phenoxazine ring, a phenothiazine ring, or an acridine ring. Note that R¹ to R¹¹, a, b, c, X¹, and X² in formulas (1′-3-1), (1′-3-2) and (1′-3-3) are defined in the same manner as those in formula (1′).

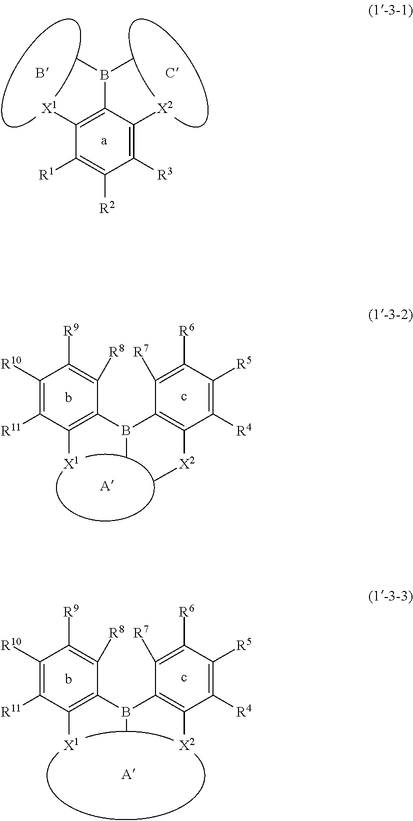

The “aryl ring” as the ring A, ring B or ring C of the general formula (1) is, for example, an aryl ring having 6 to 30 carbon atoms, and the aryl ring is preferably an aryl ring having 6 to 16 carbon atoms, more preferably an aryl ring having 6 to 12 carbon atoms, and particularly preferably an aryl ring having 6 to 10 carbon atoms. Incidentally, this “aryl ring” corresponds to the “aryl ring formed by bonding adjacent groups among R¹ to R¹¹ together with the ring a, ring b, or ring c” defined by general formula (1′). Ring a (or ring b or ring c) is already constituted by a benzene ring having 6 carbon atoms, and therefore the carbon number of 9 in total of a fused ring obtained by fusing a 5-membered ring to this benzene ring becomes a lower limit of the carbon number.

Specific examples of the “aryl ring” include a benzene ring which is a monocyclic system; a biphenyl ring which is a bicyclic system; a naphthalene ring which is a fused bicyclic system; a terphenyl ring (m-terphenyl, o-terphenyl, or p-terphenyl) which is a tricyclic system; an acenaphthylene ring, a fluorene ring, a phenalene ring and a phenanthrene ring which are fused tricyclic systems; a triphenylene ring, a pyrene ring and a naphthacene ring which are fused tetracyclic systems; and a perylene ring and a pentacene ring which are fused pentacyclic systems.

The “heteroaryl ring” as the ring A, ring B or ring C of general formula (1) is, for example, a heteroaryl ring having 2 to 30 carbon atoms, and the heteroaryl ring is preferably a heteroaryl ring having 2 to 25 carbon atoms, more preferably a heteroaryl ring having 2 to 20 carbon atoms, still more preferably a heteroaryl ring having 2 to 15 carbon atoms, and particularly preferably a heteroaryl ring having 2 to 10 carbon atoms. In addition, examples of the “heteroaryl ring” include a heterocyclic ring containing 1 to 5 heteroatoms selected from an oxygen atom, a sulfur atom, and a nitrogen atom in addition to a carbon atom as a ring-constituting atom. Incidentally, this “heteroaryl ring” corresponds to the “heteroaryl ring formed by bonding adjacent groups among the R¹ to R¹¹ together with the ring a, ring b, or ring c” defined by general formula (1′). The ring a (or ring b or ring c) is already constituted by a benzene ring having 6 carbon atoms, and therefore the carbon number of 6 in total of a fused ring obtained by fusing a 5-membered ring to this benzene ring becomes a lower limit of the carbon number.

Specific examples of the “heteroaryl ring” include a pyrrole ring, an oxazole ring, an isoxazole ring, a thiazole ring, an isothiazole ring, an imidazole ring, an oxadiazole ring, a thiadiazole ring, a triazole ring, a tetrazole ring, a pyrazole ring, a pyridine ring, a pyrimidine ring, a pyridazine ring, a pyrazine ring, a triazine ring, an indole ring, an isoindole ring, a 1H-indazole ring, a benzimidazole ring, a benzoxazole ring, a benzothiazole ring, a 1H-benzotriazole ring, a quinoline ring, an isoquinoline ring, a cinnoline ring, a quinazoline ring, a quinoxaline ring, a phthalazine ring, a naphthyridine ring, a purine ring, a pteridine ring, a carbazole ring, an acridine ring, a phenoxathiin ring, a phenoxazine ring, a phenothiazine ring, a phenazine ring, an indolizine ring, a furan ring, a benzofuran ring, an isobenzofuran ring, a dibenzofuran ring, a thiophene ring, a benzothiophene ring, a dibenzothiophene ring, a furazane ring, an oxadiazole ring, and a thianthrene ring.

At least one hydrogen atom in the above “aryl ring” or “heteroaryl ring” may be substituted by a substituted or unsubstituted “aryl”, a substituted or unsubstituted “heteroaryl”, a substituted or unsubstituted “diarylamino”, a substituted or unsubstituted “diheteroarylamino”, a substituted or unsubstituted “arylheteroarylamino”, a substituted or unsubstituted “alkyl”, a substituted or unsubstituted “alkoxy”, or a substituted or unsubstituted “aryloxy”, which is a primary substituent. Examples of the aryl of the “aryl”, “heteroaryl” and “diarylamino”, the heteroaryl of the “diheteroarylamino”, the aryl and the heteroaryl of the “arylheteroarylamino”, and the aryl of the “aryloxy” as these primary substituents include a monovalent group of the “aryl ring” or “heteroaryl ring” described above.

Furthermore, the “alkyl” as the primary substituent may be either linear or branched, and examples thereof include a linear alkyl having 1 to 24 carbon atoms and a branched alkyl having 3 to 24 carbon atoms. An alkyl having 1 to 18 carbon atoms (branched alkyl having 3 to 18 carbon atoms) is preferable, an alkyl having 1 to 12 carbon atoms (branched alkyl having 3 to 12 carbon atoms) is more preferable, an alkyl having 1 to 6 carbon atoms (branched alkyl having 3 to 6 carbon atoms) is still more preferable, and an alkyl having 1 to 4 carbon atoms (branched alkyl having 3 to 4 carbon atoms) is particularly preferable.

Specific examples of the alkyl include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl, 2-ethylbutyl, n-heptyl, l-methylhexyl, n-octyl, t-octyl, 1-methylheptyl, 2-ethylhexyl, 2-propylpentyl, n-nonyl, 2,2-dimethylheptyl, 2,6-dimethyl-4-heptyl, 3,5,5-trimethylhexyl, n-decyl, n-undecyl, 1-methyldecyl, n-dodecyl, n-tridecyl, 1-hexylheptyl, n-tetradecyl, n-pentadecyl, n-hexadecyl, n-heptadecyl, n-octadecyl, and n-eicosyl.

Furthermore, the “alkoxy” as a primary substituent may be, for example, a linear alkoxy having 1 to 24 carbon atoms or a branched alkoxy having 3 to 24 carbon atoms. The alkoxy is preferably an alkoxy having 1 to 18 carbon atoms (branched alkoxy having 3 to 18 carbon atoms), more preferably an alkoxy having 1 to 12 carbon atoms (branched alkoxy having 3 to 12 carbon atoms), still more preferably an alkoxy having 1 to 6 carbon atoms (branched alkoxy having 3 to 6 carbon atoms), and particularly preferably an alkoxy having 1 to 4 carbon atoms (branched alkoxy having 3 to 4 carbon atoms).

Specific examples of the alkoxy include a methoxy, an ethoxy, a propoxy, an isopropoxy, a butoxy, an isobutoxy, a s-butoxy, a t-butoxy, a pentyloxy, a hexyloxy, a heptyloxy, and an octyloxy.

In the substituted or unsubstituted “aryl”, substituted or unsubstituted “heteroaryl”, substituted or unsubstituted “diarylamino”, substituted or unsubstituted “diheteroarylamino”, substituted or unsubstituted “arylheteroarylamino”, substituted or unsubstituted “alkyl”, substituted or unsubstituted “alkoxy”, or substituted or unsubstituted “aryloxy”, which is the primary substituent, at least one hydrogen atom may be substituted by a secondary substituent, as described to be substituted or unsubstituted. Examples of this secondary substituent include an aryl, a heteroaryl, and an alkyl, and for the details thereof, reference can be made to the above description on the monovalent group of the “aryl ring” or “heteroaryl ring” and the “alkyl” as the primary substituent. Furthermore, regarding the aryl or heteroaryl as the secondary substituent, an aryl or heteroaryl in which at least one hydrogen atom is substituted by an aryl such as phenyl (specific examples are described above), or an alkyl such as methyl (specific examples are described above), is also included in the aryl or heteroaryl as the secondary substituent. For instance, when the secondary substituent is a carbazolyl group, a carbazolyl group in which at least one hydrogen atom at the 9-position is substituted by an aryl such as phenyl, or an alkyl such as methyl, is also included in the heteroaryl as the secondary substituent.

Examples of the aryl, the heteroaryl, the aryl of the diarylamino, the heteroaryl of the diheteroarylamino, the aryl and the heteroaryl of the arylheteroarylamino, or the aryl of the aryloxy for R¹ to R¹¹ of general formula (1′) include the monovalent groups of the “aryl ring” or “heteroaryl ring” described in general formula (1). Furthermore, regarding the alkyl or alkoxy for R¹ to R¹¹, reference can be made to the description on the “alkyl” or “alkoxy” as the primary substituent in the above description of general formula (1). In addition, the same also applies to the aryl, heteroaryl or alkyl as the substituents for these groups. Furthermore, the same also applies to the heteroaryl, diarylamino, diheteroarylamino, arylheteroarylamino, alkyl, alkoxy, or aryloxy in a case of forming an aryl ring or a heteroaryl ring by bonding adjacent groups among R¹ to R¹¹ together with the ring a, ring b or ring c, and the aryl, heteroaryl, or alkyl as a further substituent.

R of the N—R for X¹ and X² of general formula (1) represents an aryl, a heteroaryl, or an alkyl which may be substituted by the secondary substituent described above, and at least one hydrogen atom in the aryl or heteroaryl may be substituted by, for example, an alkyl. Examples of this aryl, heteroaryl or alkyl include those described above. Particularly, an aryl having 6 to 10 carbon atoms (for example, a phenyl or a naphthyl), a heteroaryl having 2 to 15 carbon atoms (for example, carbazolyl), and an alkyl having 1 to 4 carbon atoms (for example, methyl or ethyl) are preferable. This description also applies to X¹ and X² in general formula (1′).

R of the “—C(—R)₂—” as a linking group for general formula (1) represents a hydrogen atom or an alkyl, and examples of this alkyl include those described above. Particularly, an alkyl having 1 to 4 carbon atoms (for example, methyl or ethyl) is preferable. This description also applies to “—C(—R)₂—” as a linking group for general formula (1′).

Furthermore, the light emitting layer may contain a multimer having a plurality of unit structures each represented by general formula (1), and preferably a multimer having a plurality of unit structures each represented by general formula (1′). The multimer is preferably a dimer to a hexamer, more preferably a dimer to a trimer, and a particularly preferably a dimer. The multimer may be in a form having a plurality of unit structures described above in one compound, and for example, the multimer may be in a form in which a plurality of unit structures are bonded with a linking group such as a single bond, an alkylene group having 1 to 3 carbon atoms, a phenylene group, or a naphthylene group. In addition, the multimer may be in a form in which a plurality of unit structures are bonded such that any ring contained in the unit structure (ring A, ring B or ring C, or ring a, ring b or ring c) is shared by the plurality of unit structures, or may be in a form in which the unit structures are bonded such that any rings contained in the unit structures (ring A, ring B or ring C, or ring a, ring b or ring c) are fused.

Examples of such a multimer include multimer compounds represented by the following formula (1′-4), (1′-4-1), (1′-4-2), (1′-5-1) to (1′-5-4), and (1′-6). A multimer compound represented by the following formula (1′-4) corresponds to, for example, a compound represented by formula (1-423) described below. That is, to be described in connection with general formula (1′), the multimer compound includes a plurality of unit structures each represented by general formula (1′) in one compound so as to share a benzene ring as ring a. Furthermore, a multimer compound represented by the following formula (1′-4-1) corresponds to, for example, a compound represented by the following formula (1-2665). That is, to be described in connection with general formula (1′), the multimer compound includes two unit structures each represented by general formula (1′) in one compound so as to share a benzene ring as ring a. Furthermore, a multimer compound represented by the following formula (1′-4-2) corresponds to, for example, a compound represented by the following formula (1-2666). That is, to be described in connection with general formula (1′), the multimer compound includes two unit structures each represented by general formula (1′) in one compound so as to share a benzene ring as ring a. Furthermore, multimer compounds represented by the following formulas (1′-5-1) to (1′-5-4) correspond to, for example, compounds represented by the following formulas (1-421), (1-422), (1-424), and (1-425). That is, to be described in connection with general formula (1′), the multimer compound includes a plurality of unit structures each represented by general formula (1′) in one compound so as to share a benzene ring as ring b (or ring c). Furthermore, a multimer compound represented by the following formula (1′-6) corresponds to, for example, a compound represented by any one of the following formulas (1-431) to (1-435). That is, to be described in connection with general formula (1′), for example, the multimer compound includes a plurality of unit structures each represented by general formula (1′) in one compound such that a benzene ring as ring b (or ring a or ring c) of a certain unit structure and a benzene ring as ring b (or ring a or ring c) of a certain unit structure are fused. Note that each code in the following structural formulas are defined in the same manner as those in formula (1′).

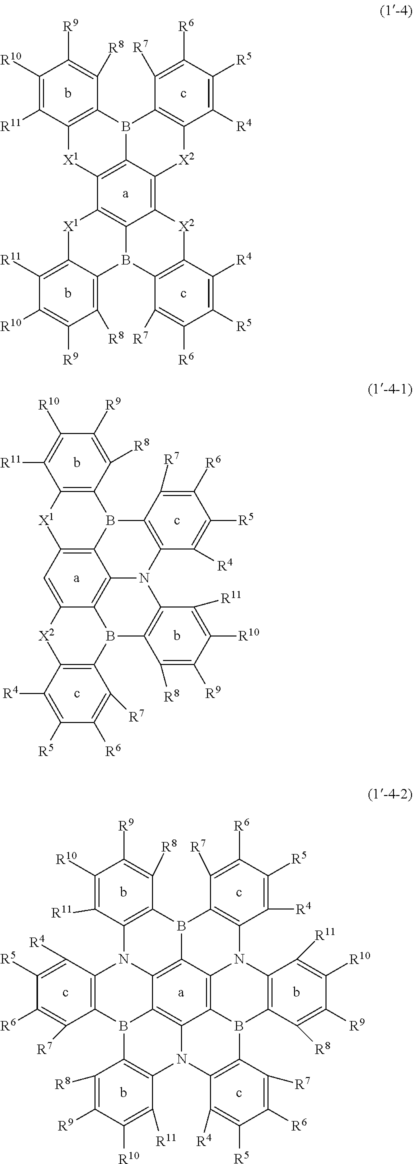

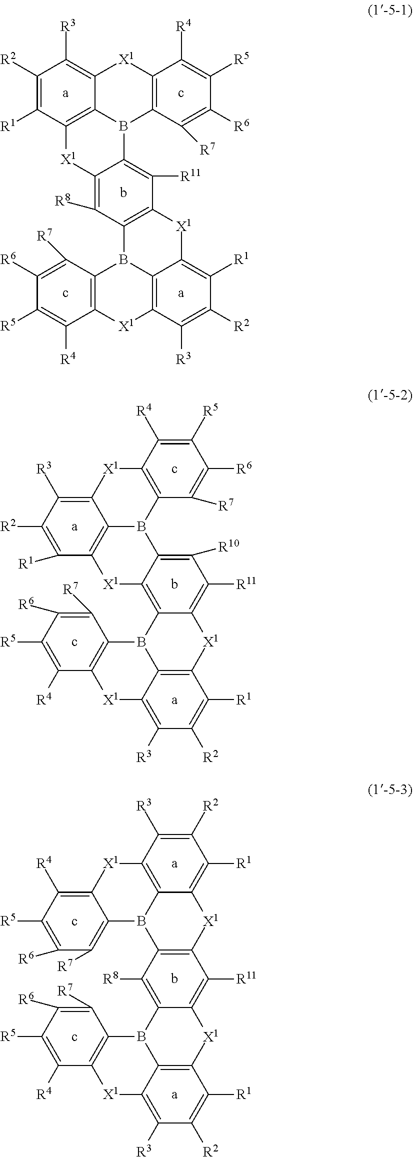

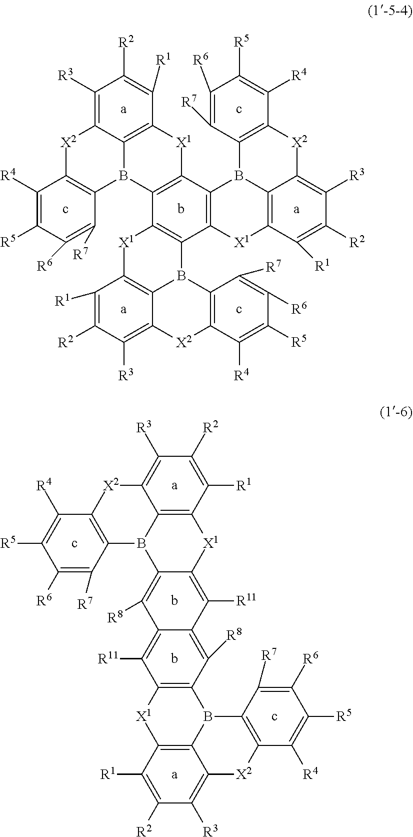

The multimer compound may be a multimer in which a multimer form represented by formula (1′-4), (1′-4-1) or (1′-4-2) and a multimer form represented by any one of formula (1′-5-1) to (1′-5-4) or (1′-6) are combined, may be (16) a multimer in which a multimer form represented by any one of formula (1′-5-1) to (1′-5-4) and a multimer form represented by formula (1′-6) are combined, or may be a multimer in which a multimer form represented by formula (1′-4), (1′-4-1) or (1′-4-2), a multimer form represented by any one of formulas (1′-5-1) to (1′-5-4), and a multimer form represented by formula (1′-6) are combined.

Furthermore, all or a portion of the hydrogen atoms in the chemical structures of the compound represented by general formula (1) or (1′) and a multimer thereof may be substituted by halogen atoms, cyanos or deuterium atoms. For example, in regard to formula (1), the hydrogen atoms in the ring A, ring B, ring C (ring A to ring C are aryl rings or heteroaryl rings), substituents on the ring A to ring C, and R (=alkyl or aryl) when X¹ and X² each represent N—R, may be substituted by halogen atoms, cyanos or deuterium atoms and among these, a form in which all or a portion of the hydrogen atoms in the aryl or heteroaryl are substituted by halogen atoms, cyanos or deuterium atoms may be mentioned. The halogen is fluorine, chlorine, bromine, or iodine, preferably fluorine, chlorine, or bromine, and more preferably chlorine.

More specific examples of the compound represented by formula (1) and a multimer thereof include compounds represented by the following structural formulas. Note that, in each structural formula, “Me” is a methyl group, “tBu” is a t-butyl group, “iPr” is an isopropyl group, and “Ph” is a phenyl group.

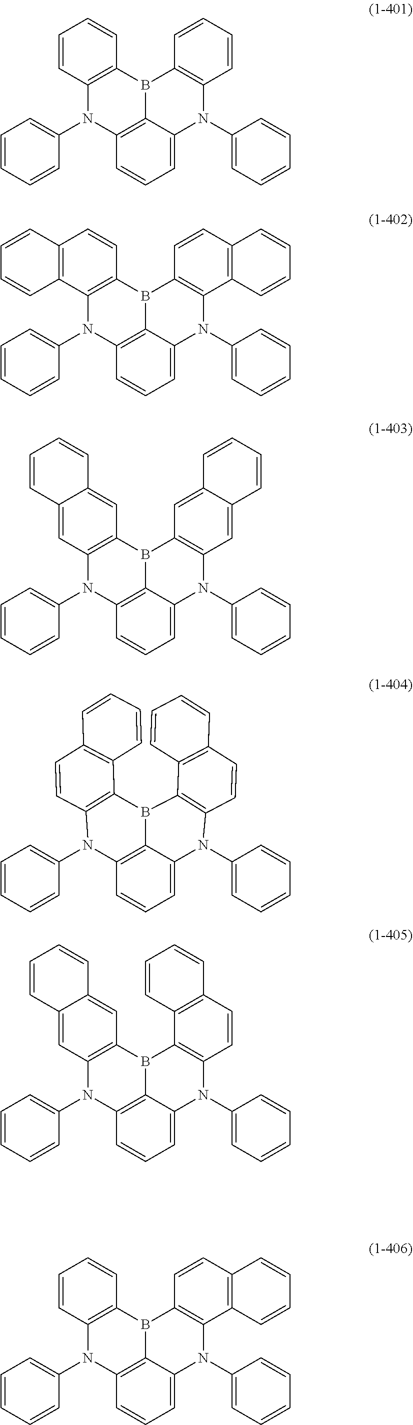

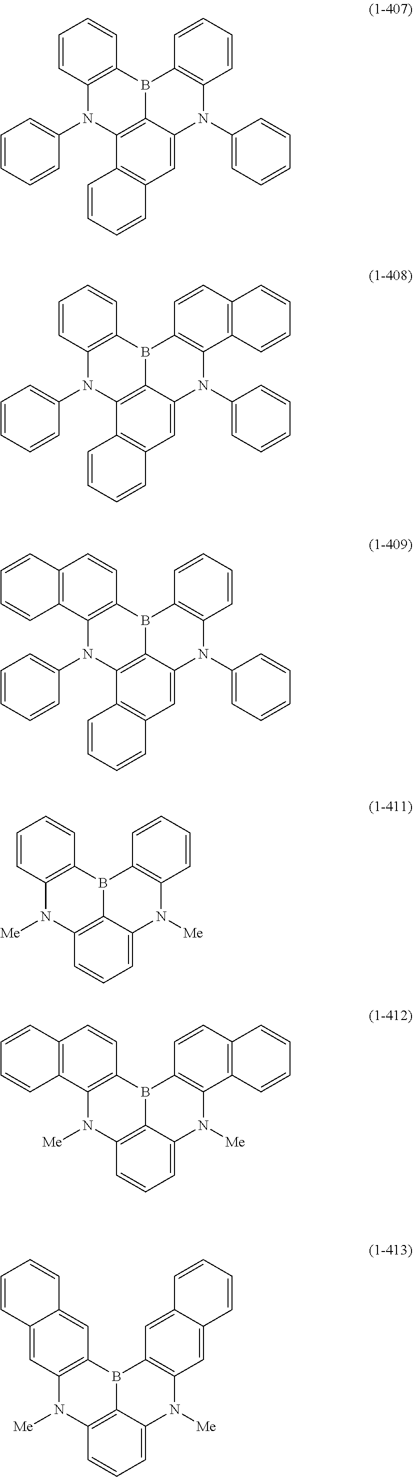

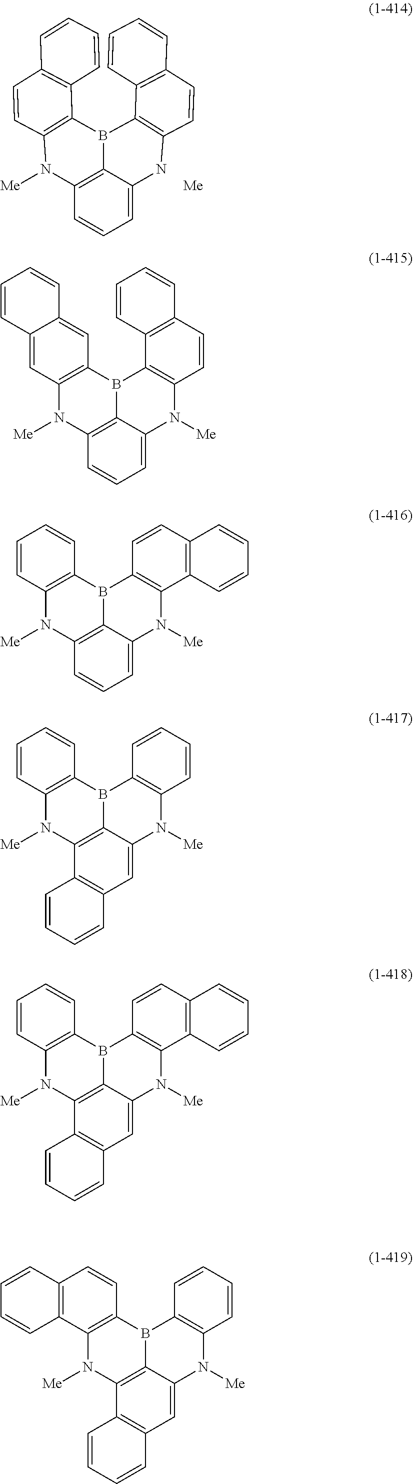

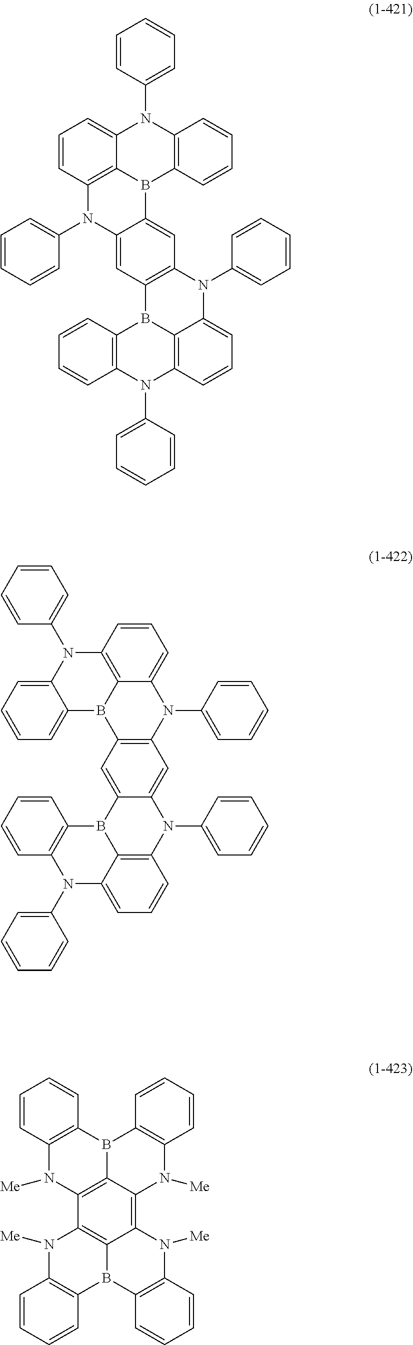 [SMILES: c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1 ; CN1c2ccccc2B2c3ccccc3N(C)c3c2c1c1c2c3N(C)c3ccccc3B2c2ccccc2N1C]

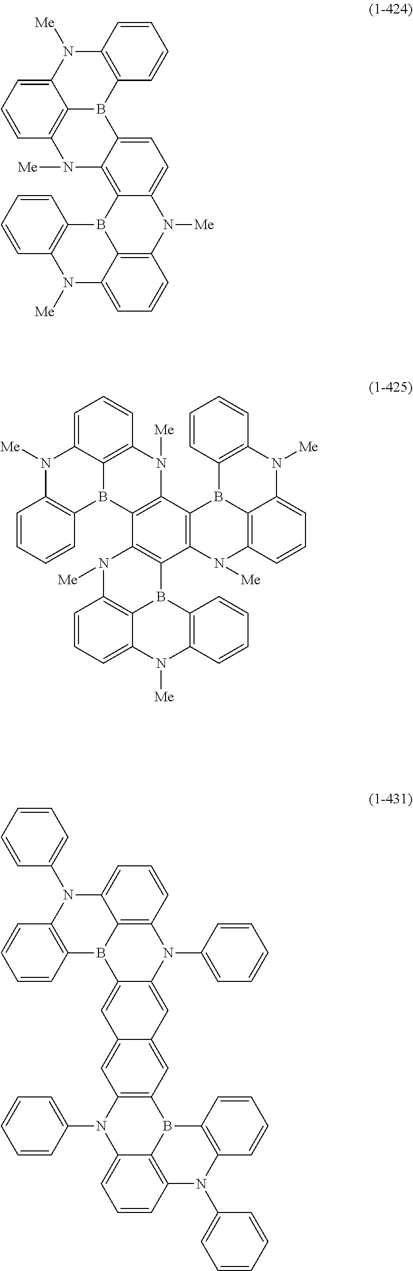 [SMILES: CN1c2ccccc2B2c3c1cccc3N(C)c1ccc3c(c12)N(C)c1cccc2c1B3c1ccccc1N2C ; CN1c2ccccc2B2c3c1cccc3N(C)c1c3c(c4c(c12)N(C)c1cccc2c1B4c1ccccc1N2C)N(C)c1cccc2c1B3c1ccccc1N2C ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1]

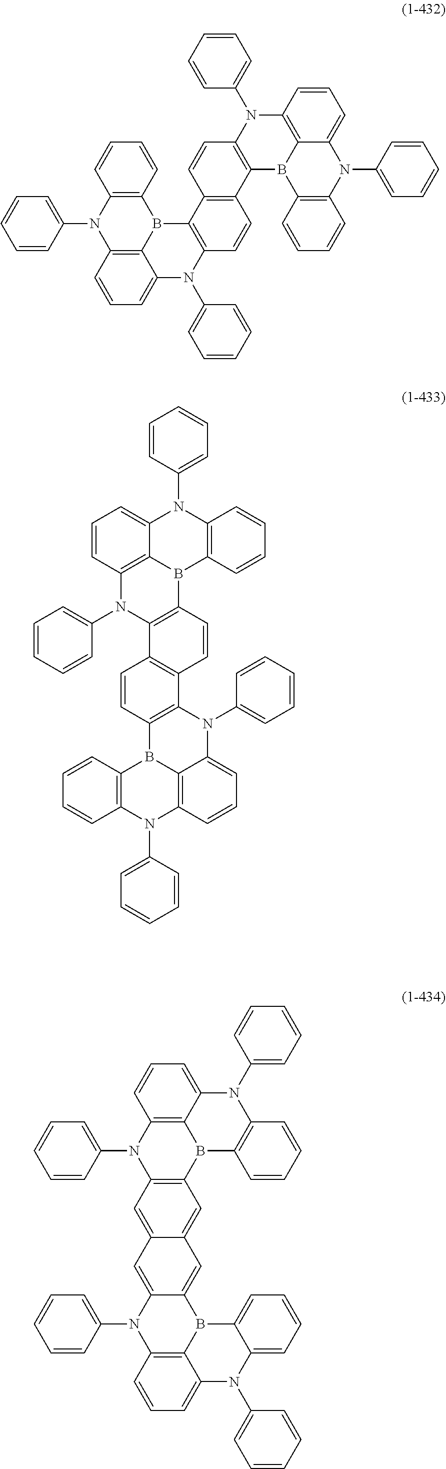 [SMILES: c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1 ; c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

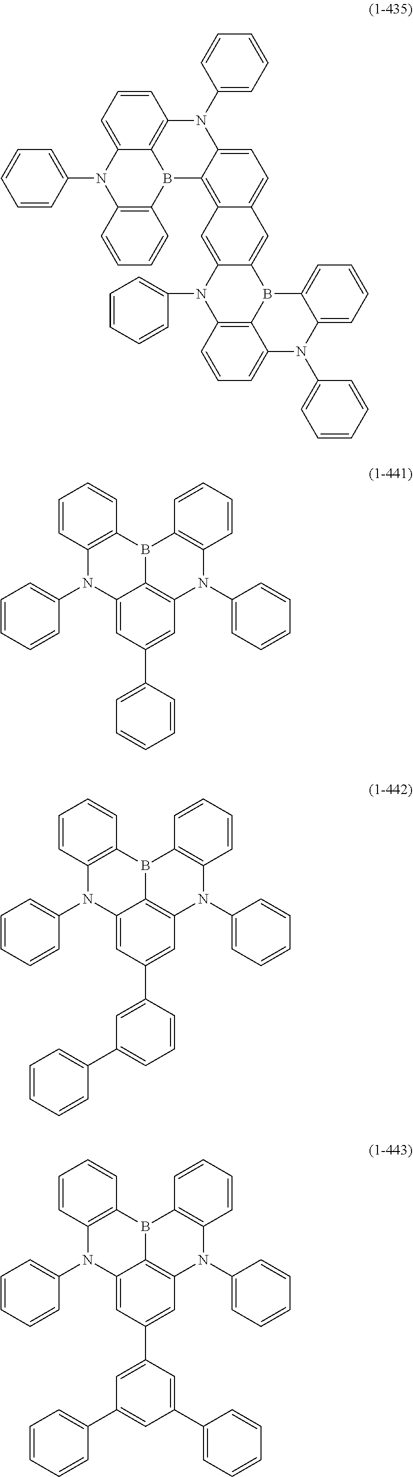 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc(-c2cc3c4c(c2)N(c2ccccc2)c2ccccc2B4c2ccccc2N3c2ccccc2)cc1 ; c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1]

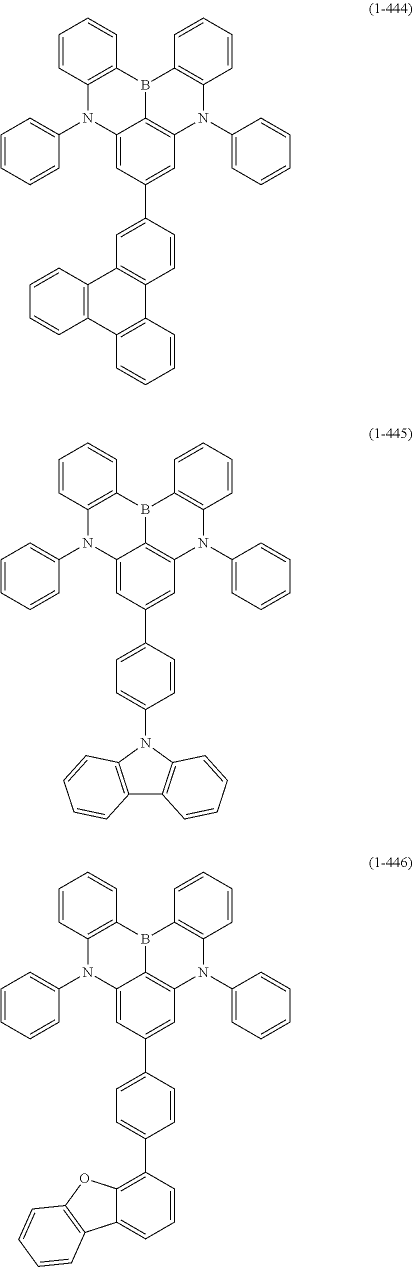 [SMILES: c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1]

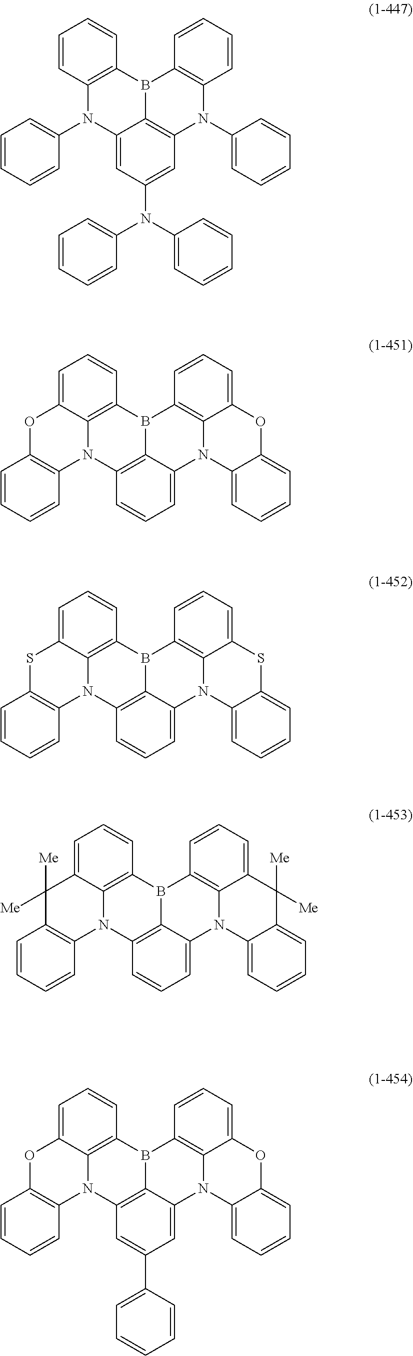 [SMILES: c1ccc(N(c2ccccc2)c2cc3c4c(c2)N(c2ccccc2)c2ccccc2B4c2ccccc2N3c2ccccc2)cc1 ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1]

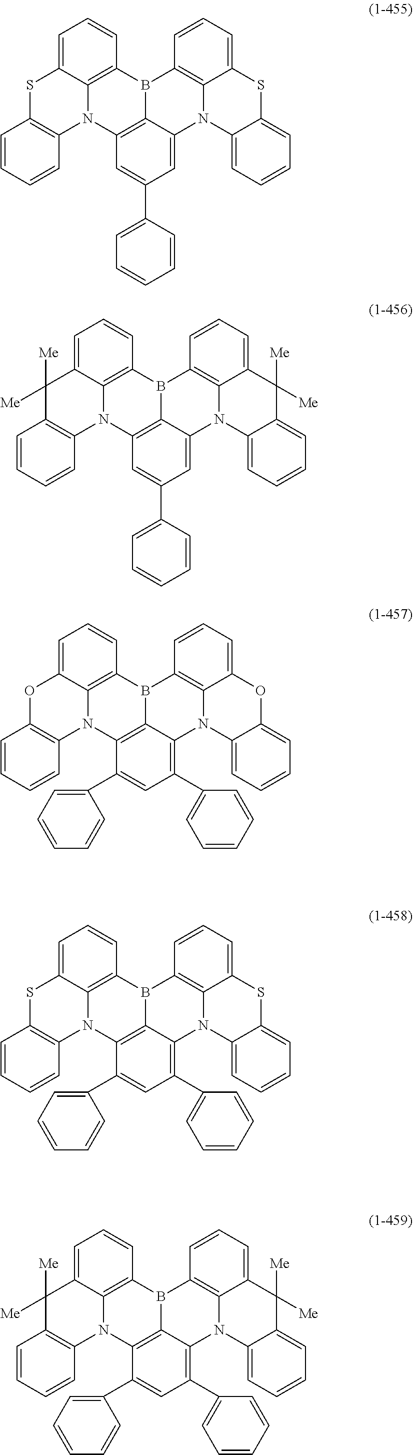 [SMILES: c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C ; c1ccc(-c2cc(-c3ccccc3)c3c4c2N2c5ccccc5Oc5cccc(c52)B4c2cccc4c2N3c2ccccc2O4)cc1 ; c1ccc(-c2cc(-c3ccccc3)c3c4c2N2c5ccccc5Sc5cccc(c52)B4c2cccc4c2N3c2ccccc2S4)cc1 ; CC1(C)c2ccccc2N2c3c(cccc31)B1c3cccc4c3N(c3ccccc3C4(C)C)c3c(-c4ccccc4)cc(-c4ccccc4)c2c31]

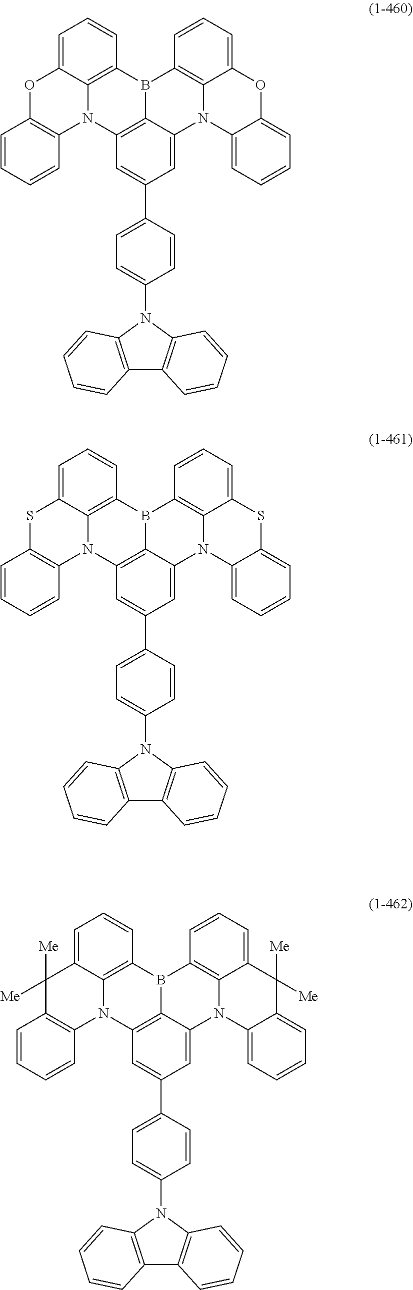 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C]

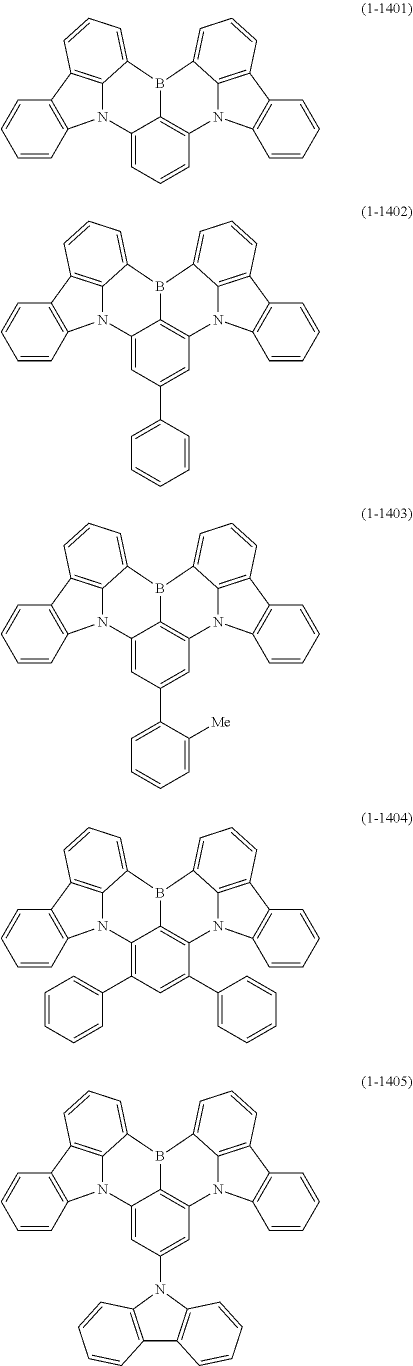 [SMILES: c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13 ; c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13]

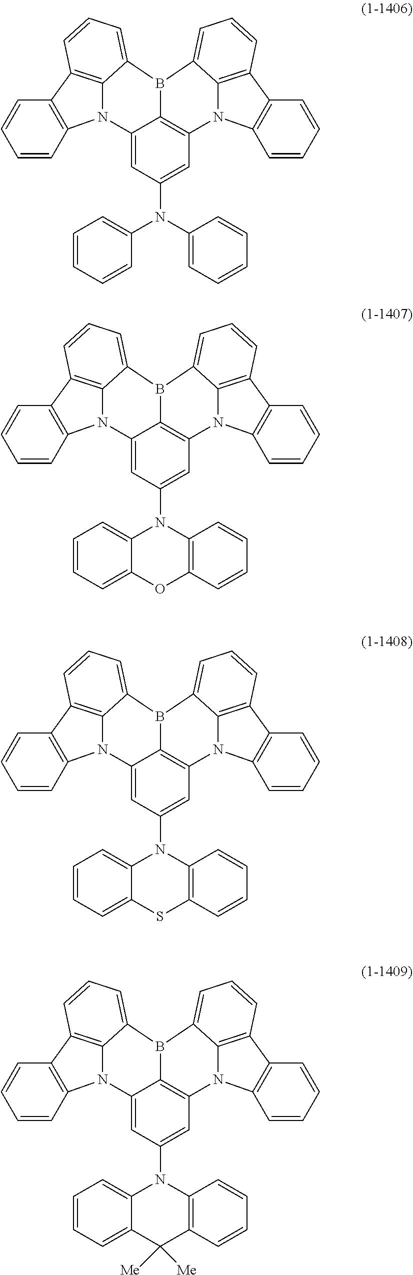

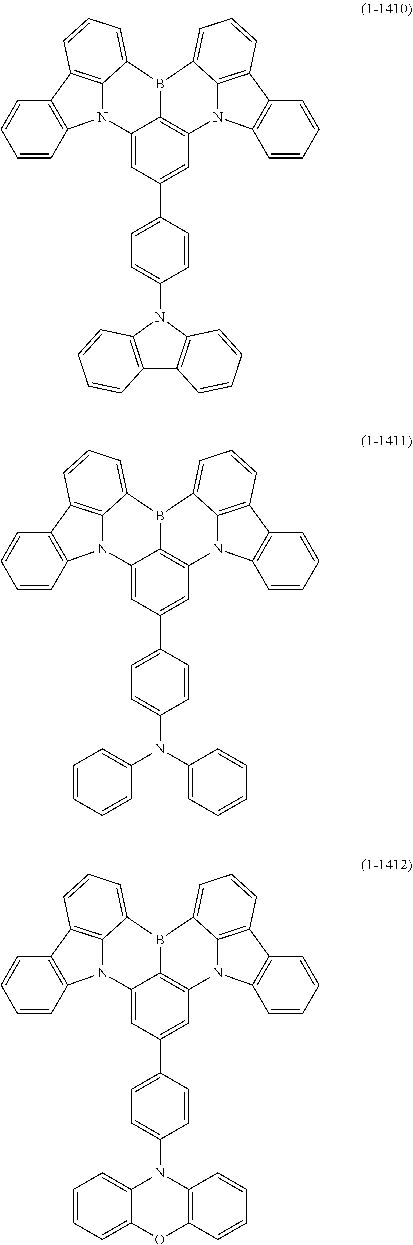 [SMILES: c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1]

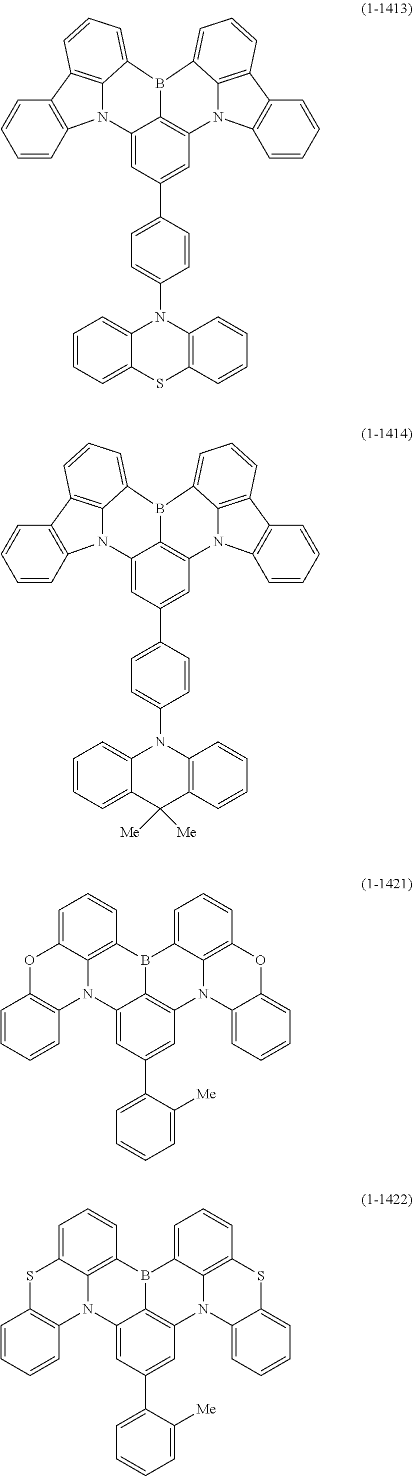 [SMILES: c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3]

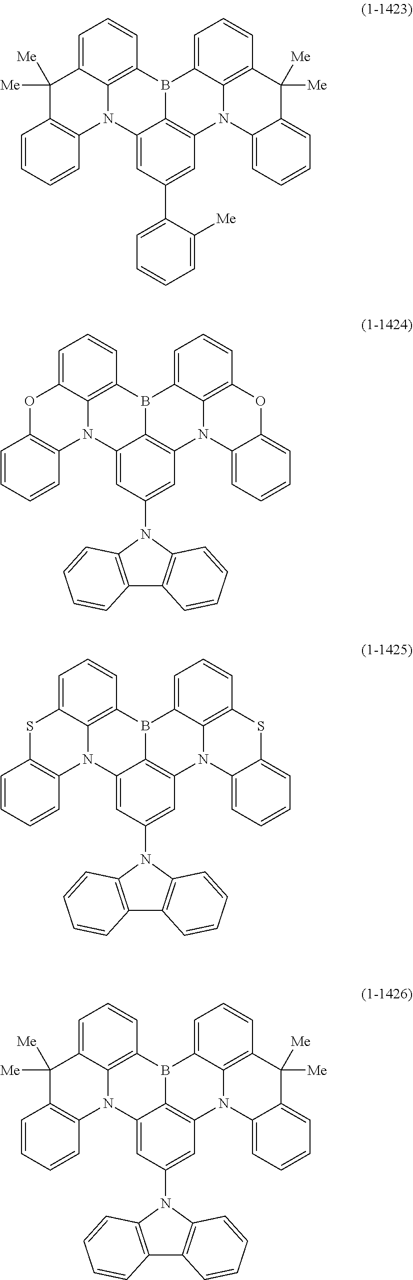 [SMILES: Cc1ccccc1-c1cc2c3c(c1)N1c4ccccc4C(C)(C)c4cccc(c41)B3c1cccc3c1N2c1ccccc1C3(C)C ; c1ccc2c(c1)Oc1cccc3c1N2c1cc(-n2c4ccccc4c4ccccc42)cc2c1B3c1cccc3c1N2c1ccccc1O3 ; c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C]

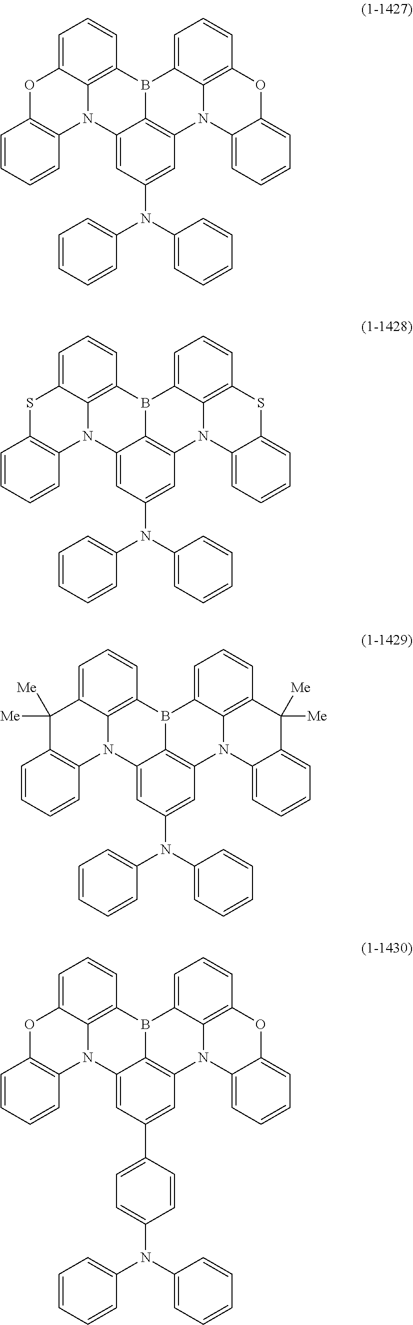

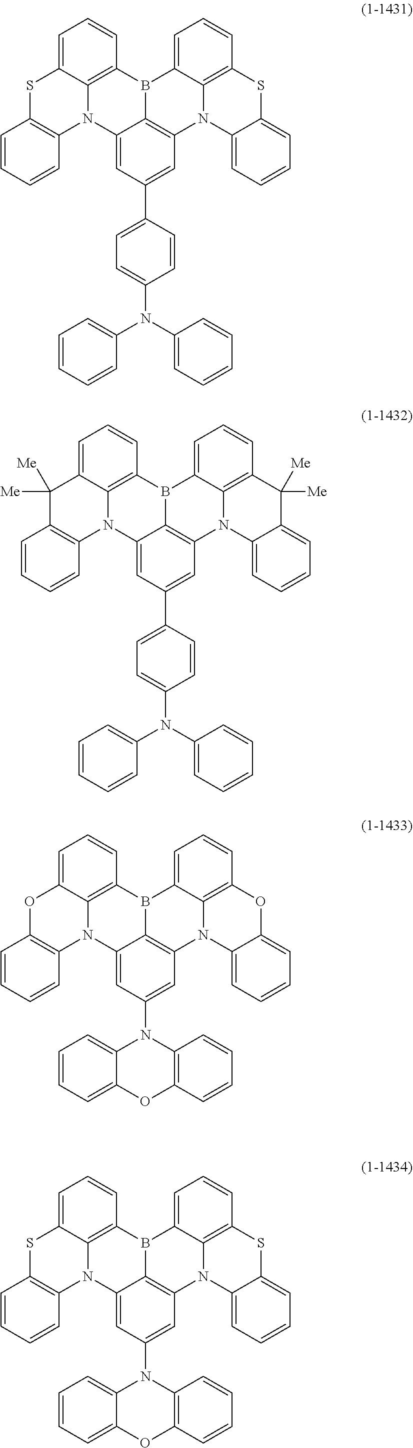

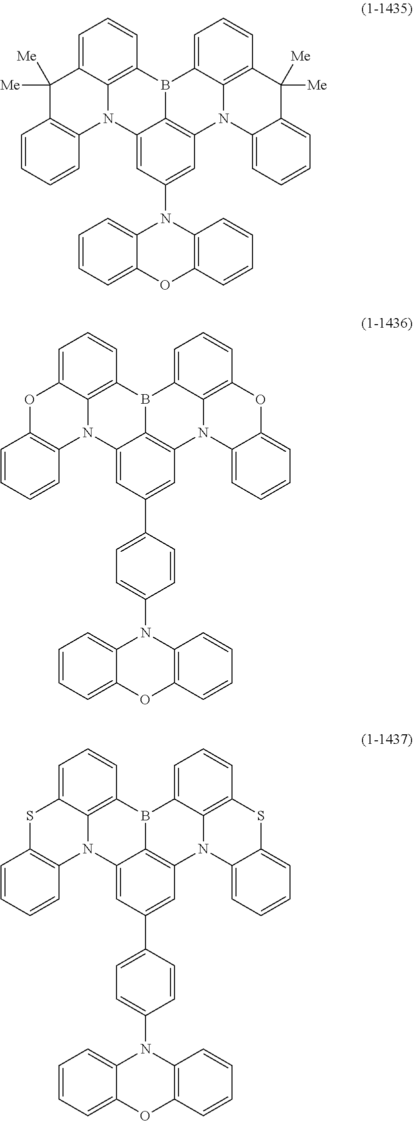 [SMILES: CC1(C)c2ccccc2N2c3cc(N4c5ccccc5Oc5ccccc54)cc4c3B(c3cccc1c32)c1cccc2c1N4c1ccccc1C2(C)C ; c1ccc2c(c1)Oc1ccccc1N2c1ccc(-c2cc3c4c(c2)N2c5ccccc5Oc5cccc(c52)B4c2cccc4c2N3c2ccccc2O4)cc1 ; c1ccc2c(c1)Oc1ccccc1N2c1ccc(-c2cc3c4c(c2)N2c5ccccc5Sc5cccc(c52)B4c2cccc4c2N3c2ccccc2S4)cc1]

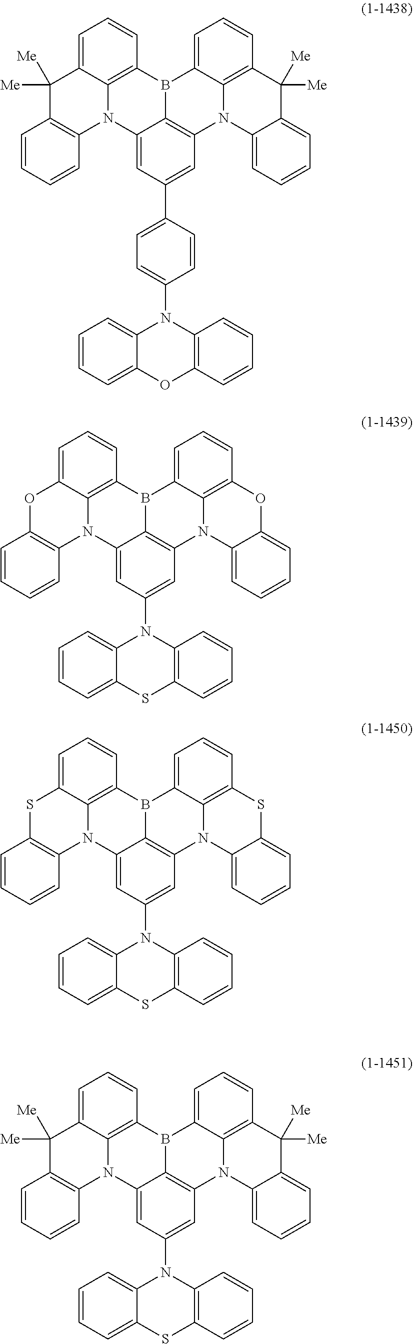

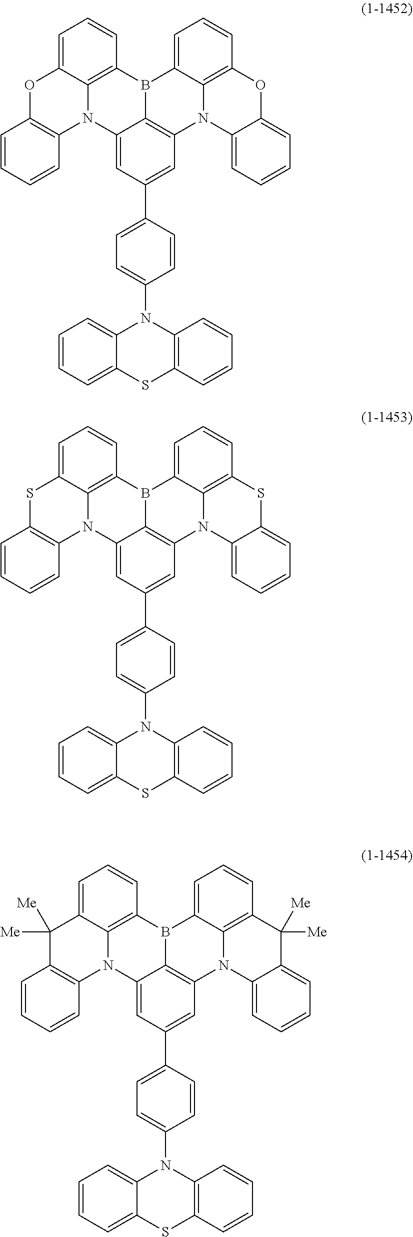

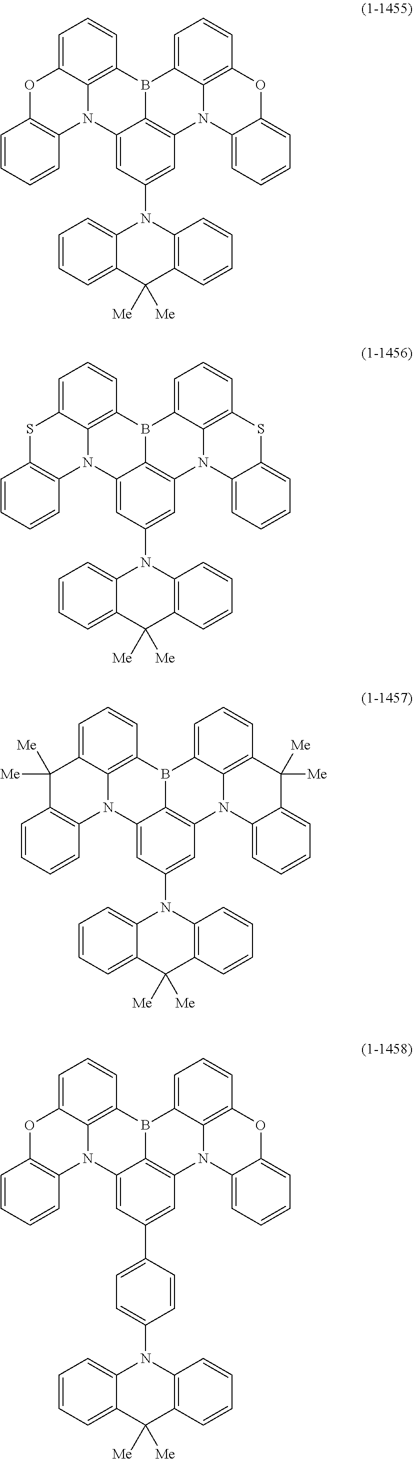 [SMILES: CC1(C)c2ccccc2N(c2cc3c4c(c2)N2c5ccccc5Oc5cccc(c52)B4c2cccc4c2N3c2ccccc2O4)c2ccccc21 ; CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

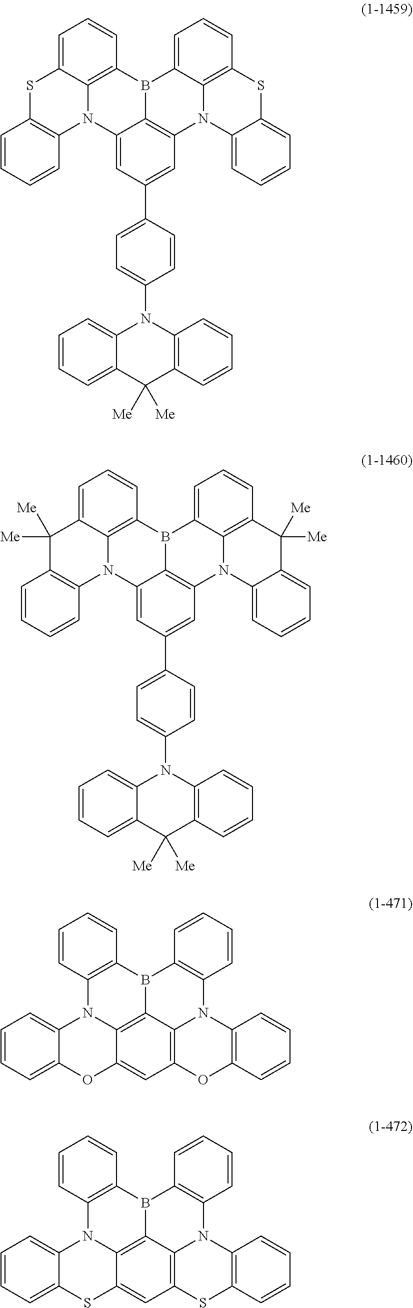 [SMILES: CC1(C)c2ccccc2N(c2ccc(-c3cc4c5c(c3)N3c6ccccc6Sc6cccc(c63)B5c3cccc5c3N4c3ccccc3S5)cc2)c2ccccc21 ; CC1(C)c2ccccc2N(c2ccc(-c3cc4c5c(c3)N3c6ccccc6C(C)(C)c6cccc(c63)B5c3cccc5c3N4c3ccccc3C5(C)C)cc2)c2ccccc21 ; c1ccc2c(c1)B1c3ccccc3-n3c4ccccc4oc4cc5oc6ccccc6n-2c5c1c43 ; c1ccc2c(c1)B1c3ccccc3-n3c4ccccc4sc4cc5sc6ccccc6n-2c5c1c43]

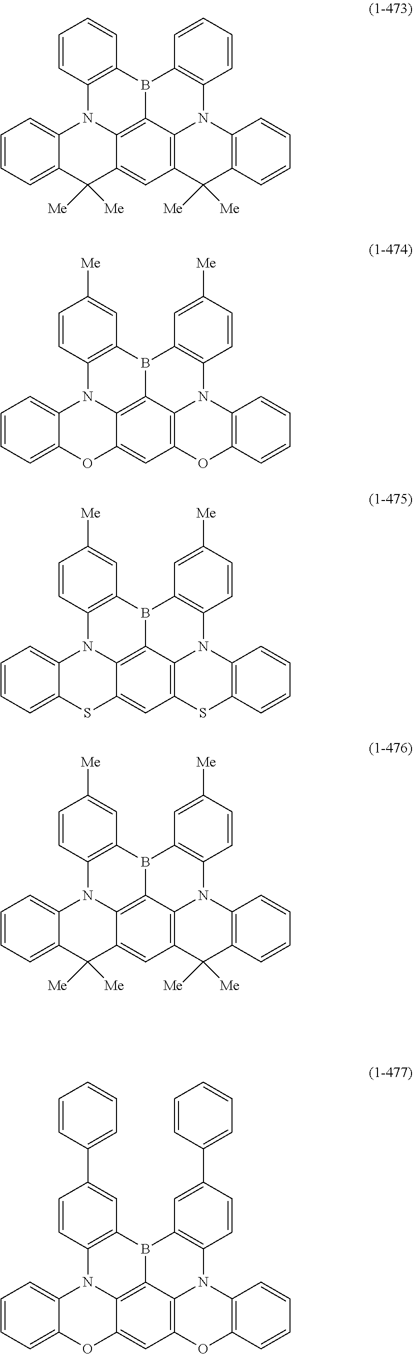 [SMILES: CC1(C)c2ccccc2N2c3ccccc3B3c4ccccc4N4c5ccccc5C(C)(C)c5cc1c2c3c54 ; Cc1ccc2c(c1)B1c3cc(C)ccc3-n3c4ccccc4oc4cc5oc6ccccc6n-2c5c1c43 ; Cc1ccc2c(c1)B1c3cc(C)ccc3-n3c4ccccc4sc4cc5sc6ccccc6n-2c5c1c43 ; Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1]

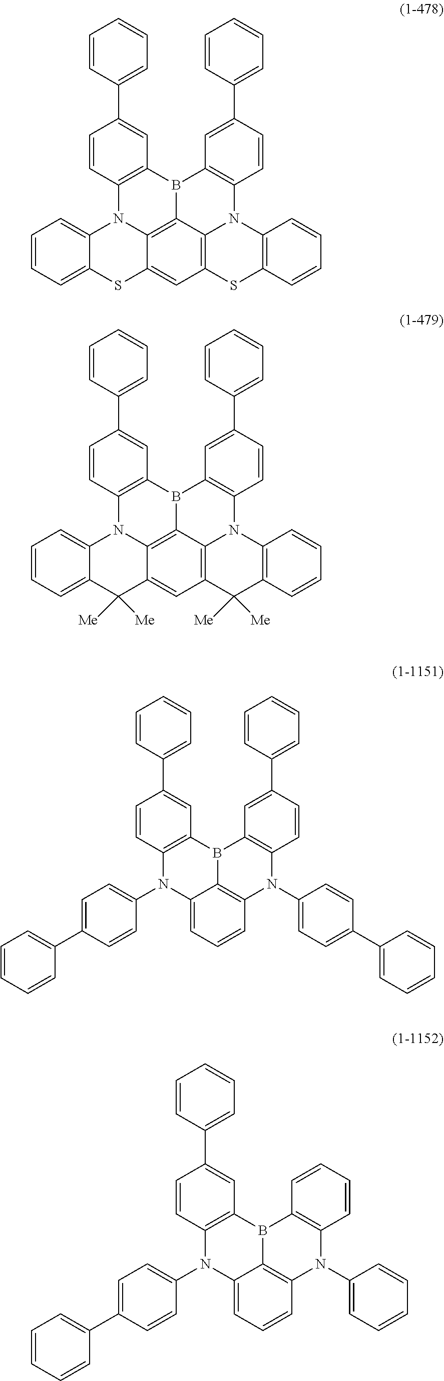

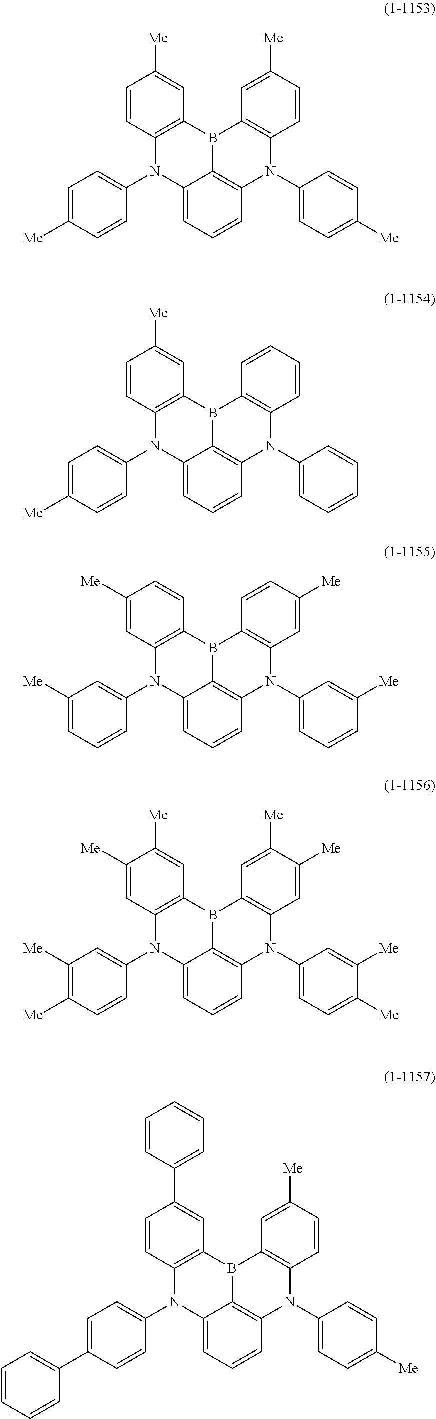 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1]

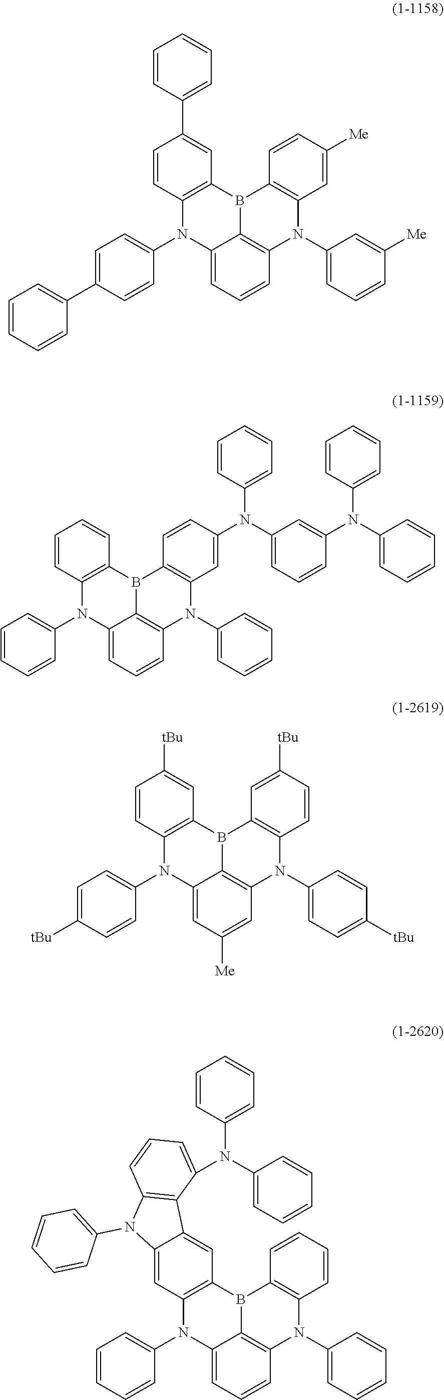 [SMILES: Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

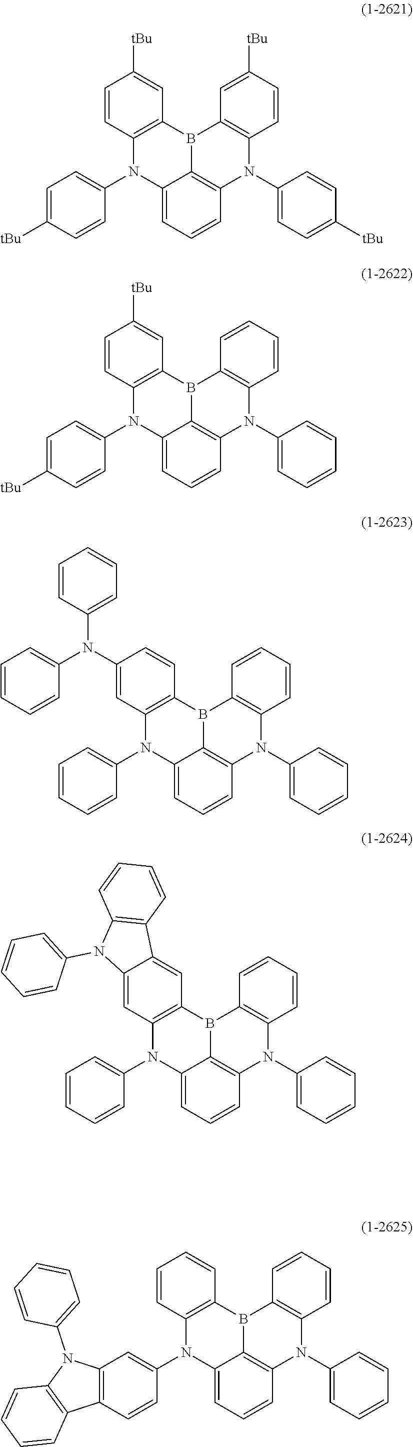 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1]

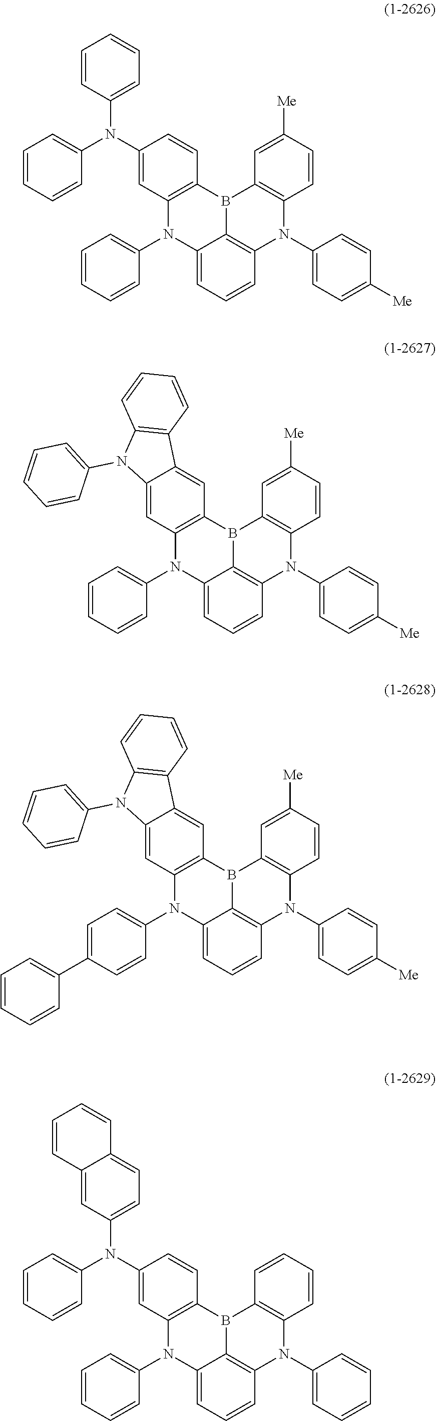 [SMILES: Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3ccccc3c2)cc1]

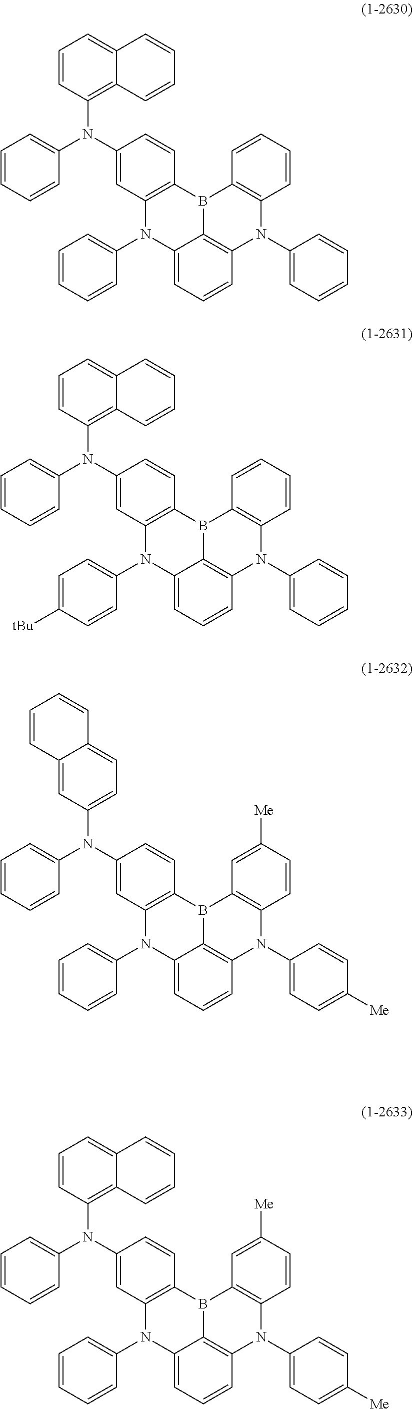 [SMILES: c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccc(C)cc3B3c4ccc(N(c5ccccc5)c5cccc6ccccc56)cc4N(c4ccccc4)c4cccc2c43)cc1]

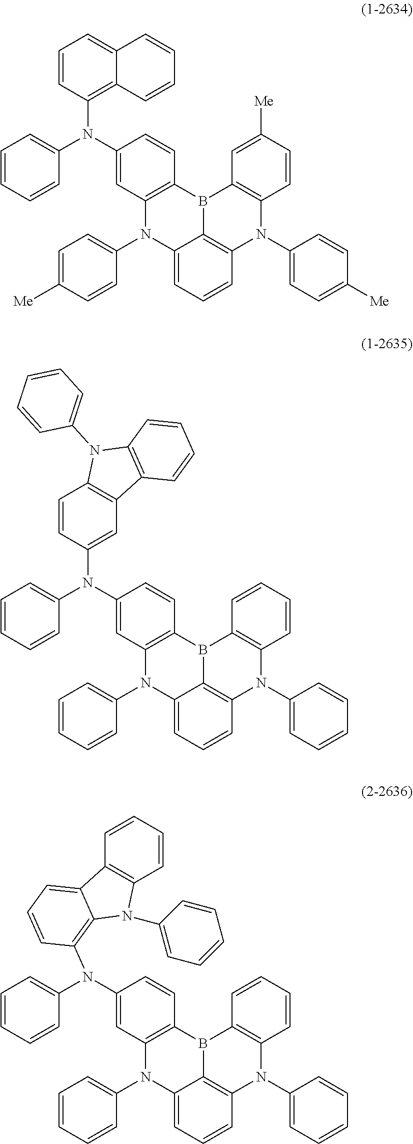 [SMILES: Cc1ccc(N2c3ccc(C)cc3B3c4ccc(N(c5ccccc5)c5cccc6ccccc56)cc4N(c4ccc(C)cc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

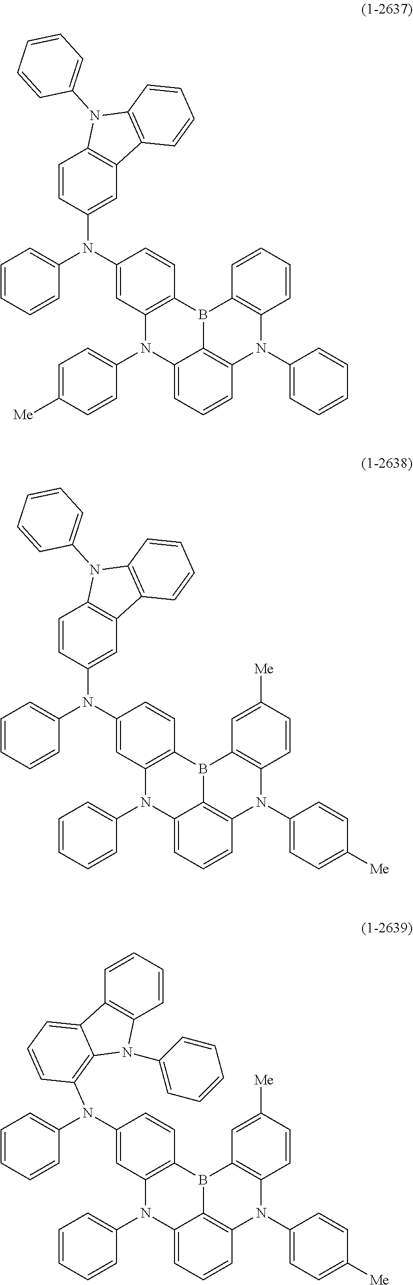 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

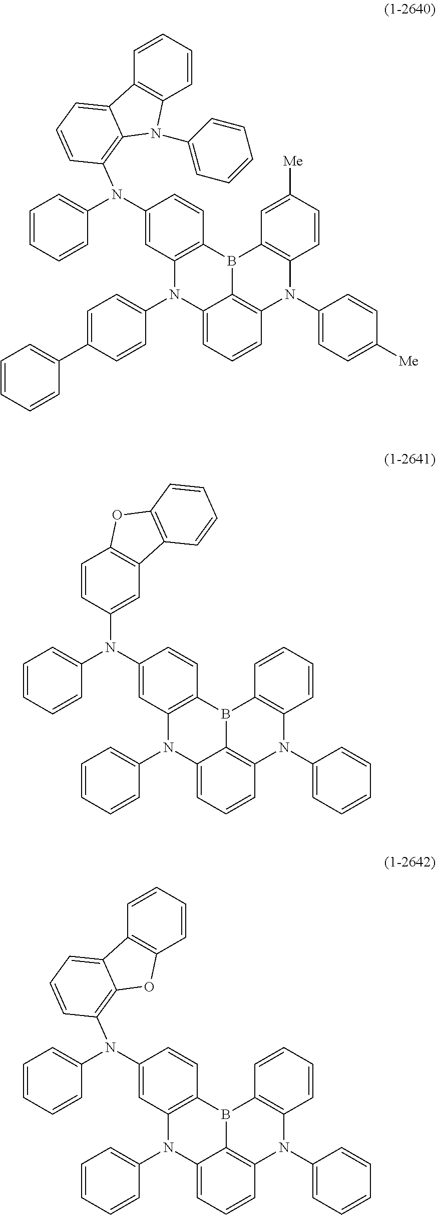 [SMILES: Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1]

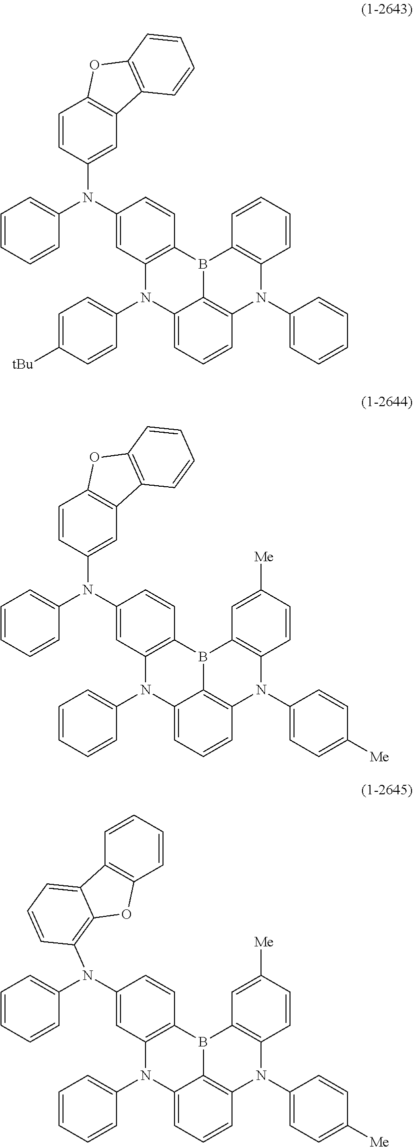 [SMILES: CC(C)(C)c1ccc(N2c3cc(N(c4ccccc4)c4ccc5oc6ccccc6c5c4)ccc3B3c4ccccc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1]

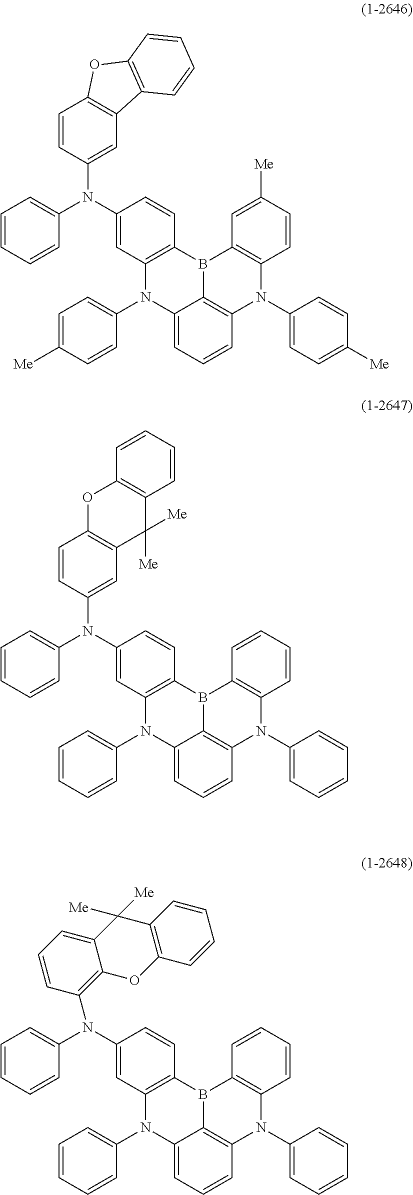 [SMILES: Cc1ccc(N2c3ccc(C)cc3B3c4ccc(N(c5ccccc5)c5ccc6oc7ccccc7c6c5)cc4N(c4ccc(C)cc4)c4cccc2c43)cc1 ; CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21]

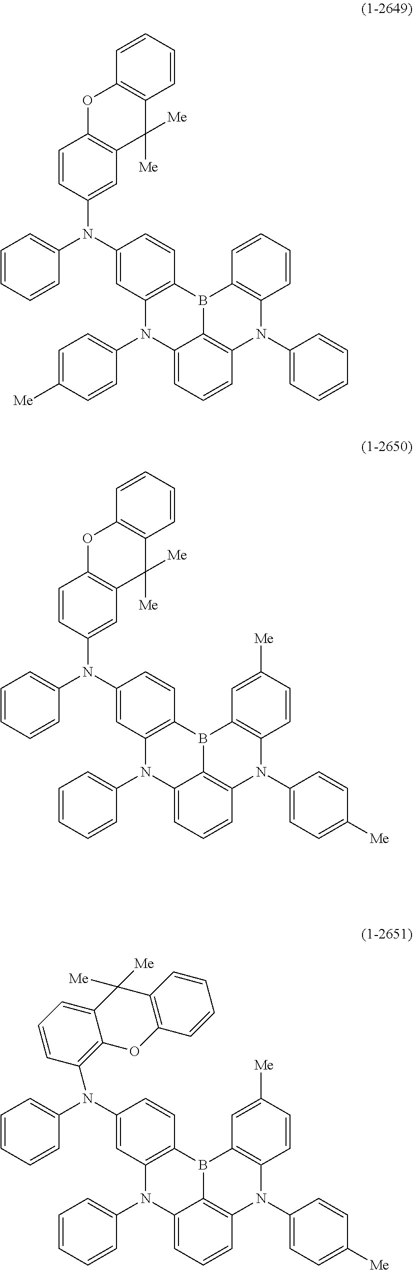 [SMILES: Cc1ccc(N2c3cc(N(c4ccccc4)c4ccc5c(c4)C(C)(C)c4ccccc4O5)ccc3B3c4ccccc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1]

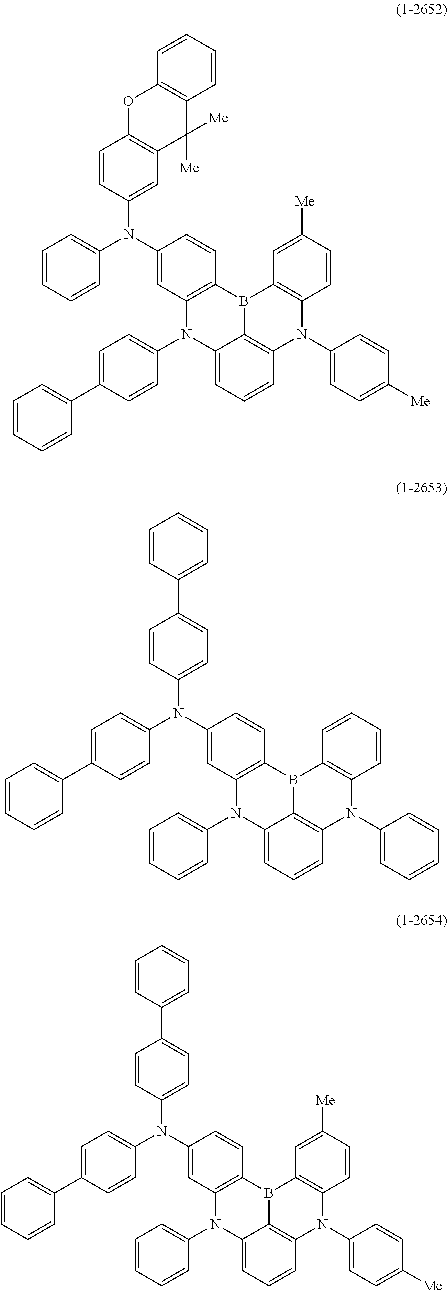 [SMILES: Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1 ; c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

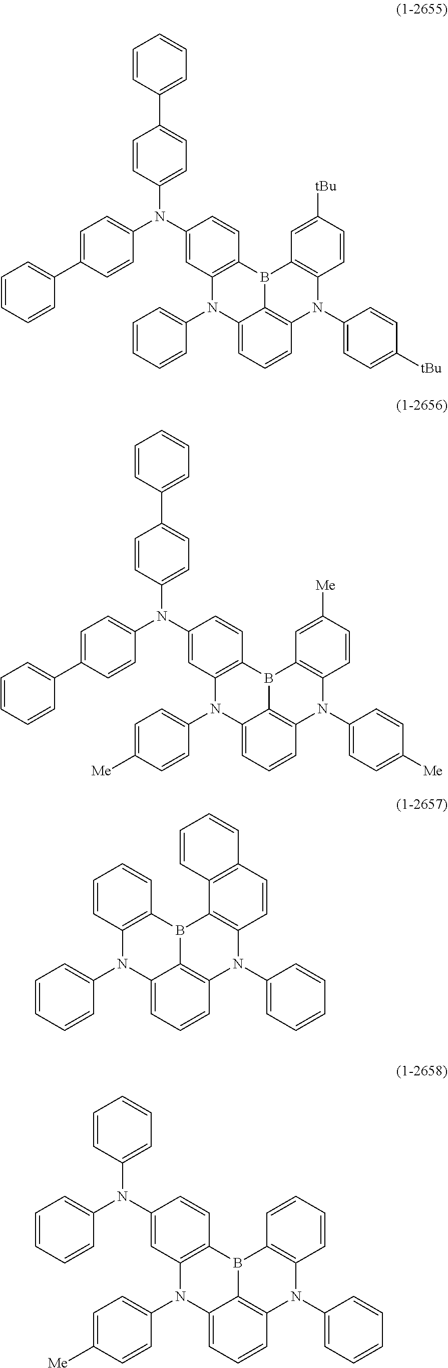 [SMILES: CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1]

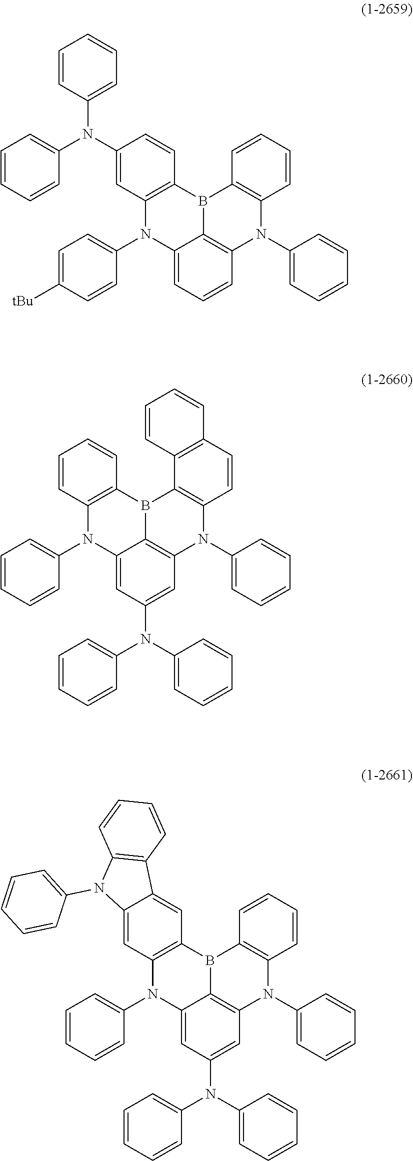 [SMILES: CC(C)(C)c1ccc(N2c3cc(N(c4ccccc4)c4ccccc4)ccc3B3c4ccccc4N(c4ccccc4)c4cccc2c43)cc1 ; c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1]

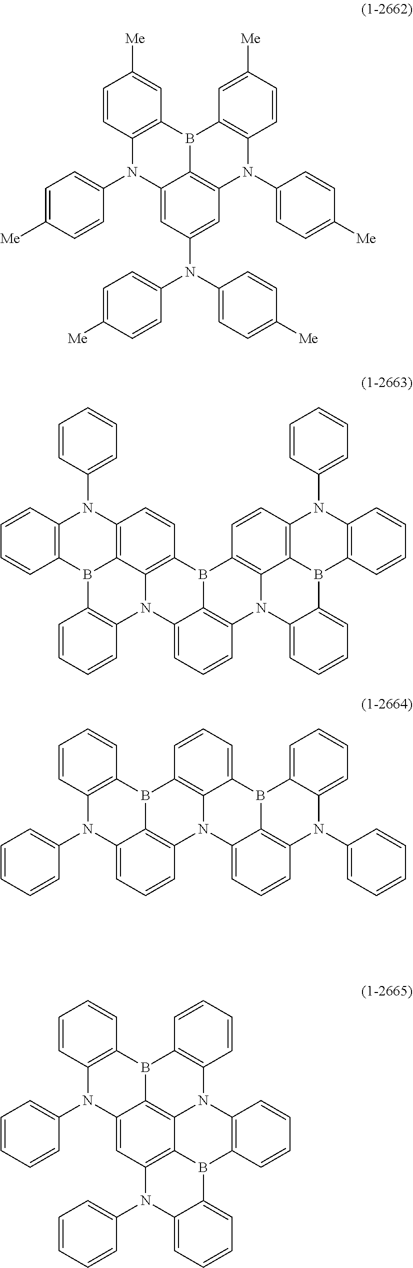 [SMILES: Cc1ccc(N(c2ccc(C)cc2)c2cc3c4c(c2)N(c2ccc(C)cc2)c2ccc(C)cc2B4c2cc(C)ccc2N3c2ccc(C)cc2)cc1 ; c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1 ; c1ccc(N2c3ccccc3B3c4ccccc4N4c5ccccc5B5c6ccccc6N(c6ccccc6)c6cc2c3c4c65)cc1]

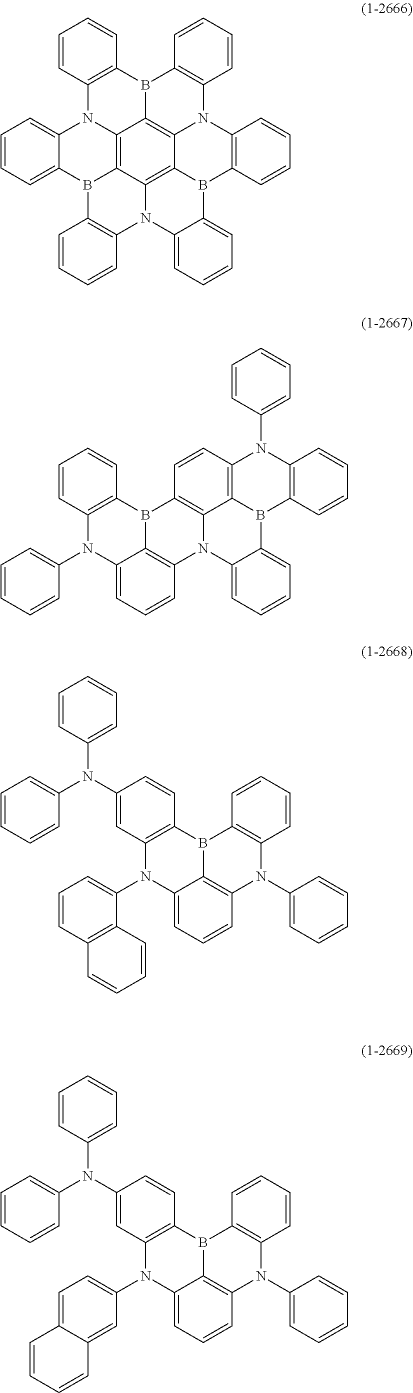 [SMILES: c1ccc2c(c1)B1c3ccccc3N3c4ccccc4B4c5ccccc5N5c6ccccc6B6c7ccccc7N2c2c1c3c4c5c26 ; c1ccc(N2c3ccccc3B3c4ccc5c6c4N(c4ccccc4B6c4ccccc4N5c4ccccc4)c4cccc2c43)cc1 ; c1ccc(N(c2ccccc2)c2ccc3c(c2)N(c2cccc4ccccc24)c2cccc4c2B3c2ccccc2N4c2ccccc2)cc1 ; c1ccc(N(c2ccccc2)c2ccc3c(c2)N(c2ccc4ccccc4c2)c2cccc4c2B3c2ccccc2N4c2ccccc2)cc1]

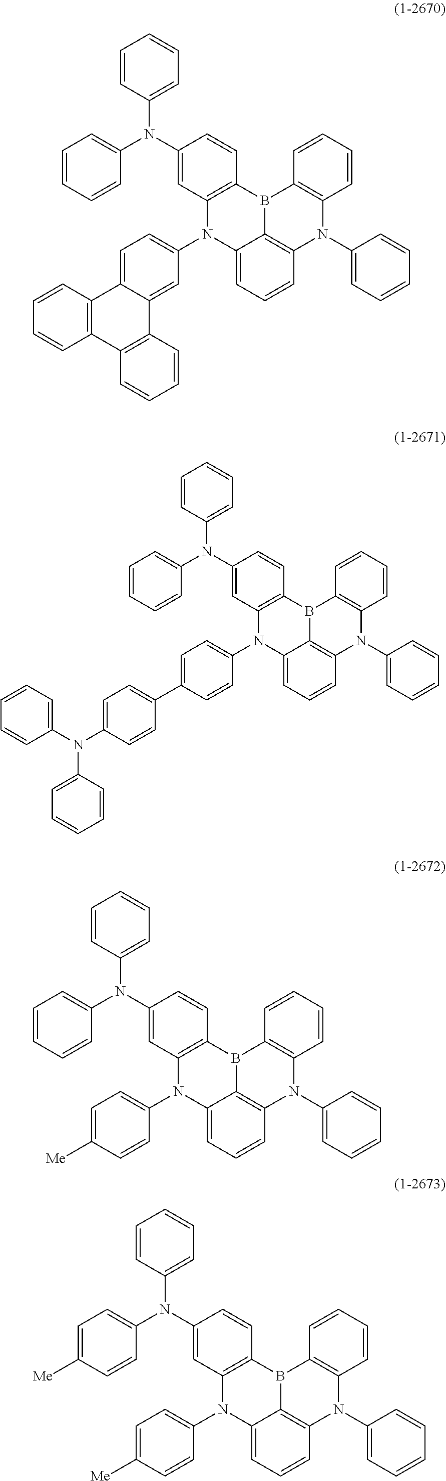 [SMILES: c1ccc(N(c2ccccc2)c2ccc3c(c2)N(c2ccc4c5ccccc5c5ccccc5c4c2)c2cccc4c2B3c2ccccc2N4c2ccccc2)cc1 ; c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1 ; Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1]

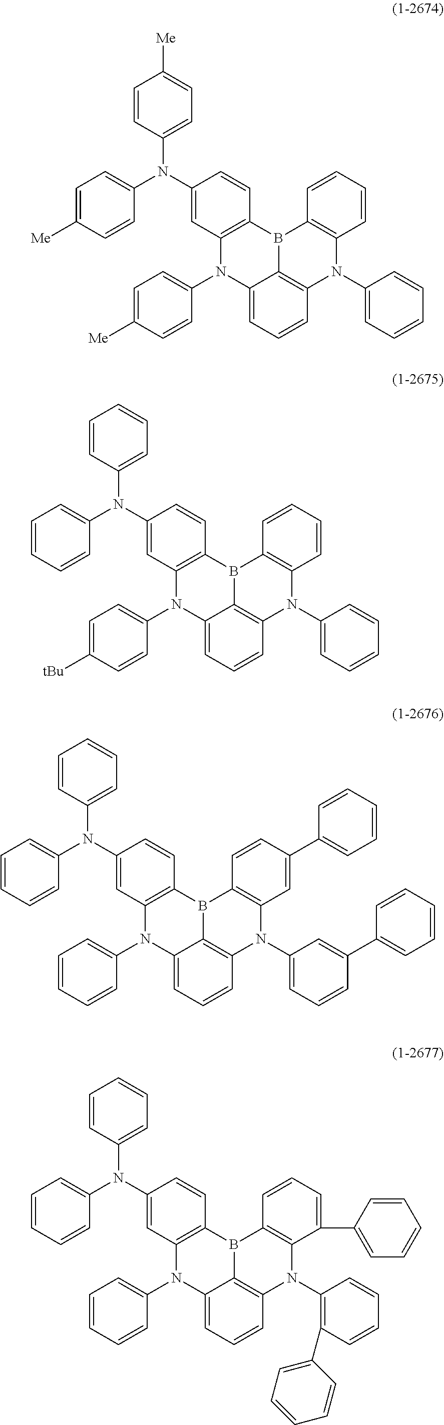 [SMILES: Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1]

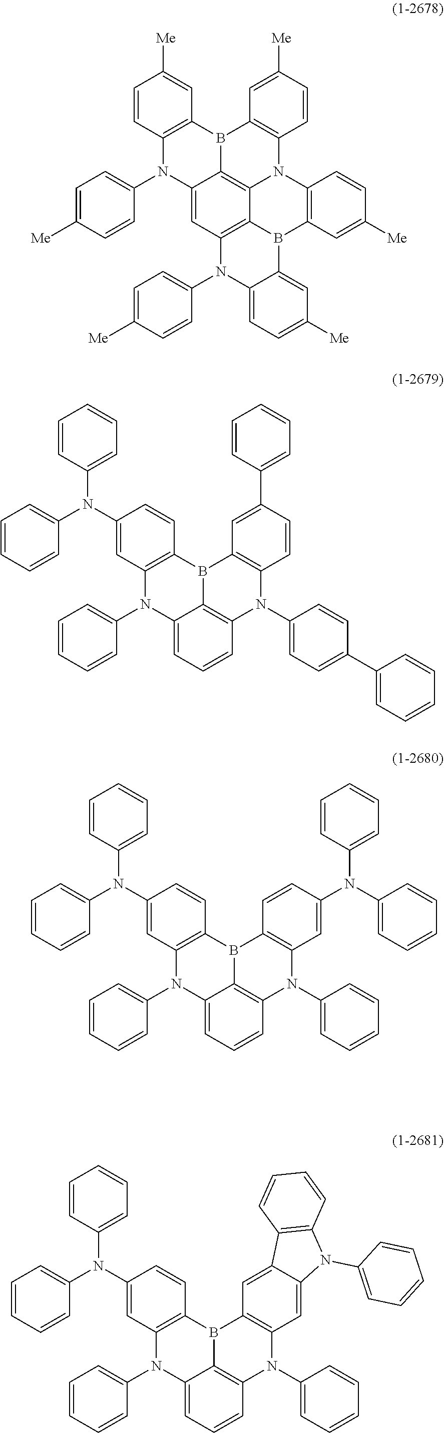 [SMILES: Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1]

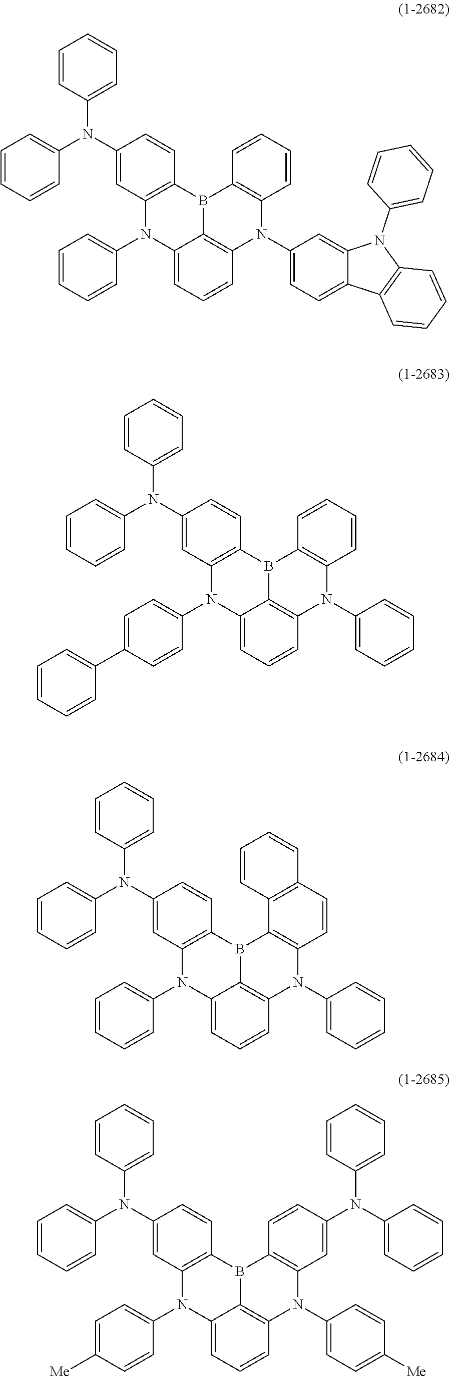 [SMILES: c1ccc(N(c2ccccc2)c2ccc3c(c2)N(c2ccccc2)c2cccc4c2B3c2ccccc2N4c2ccc3c4ccccc4n(-c4ccccc4)c3c2)cc1 ; c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2c(ccc3ccccc23)N4c2ccccc2)cc1 ; Cc1ccc(N2c3cc(N(c4ccccc4)c4ccccc4)ccc3B3c4ccc(N(c5ccccc5)c5ccccc5)cc4N(c4ccc(C)cc4)c4cccc2c43)cc1]

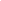

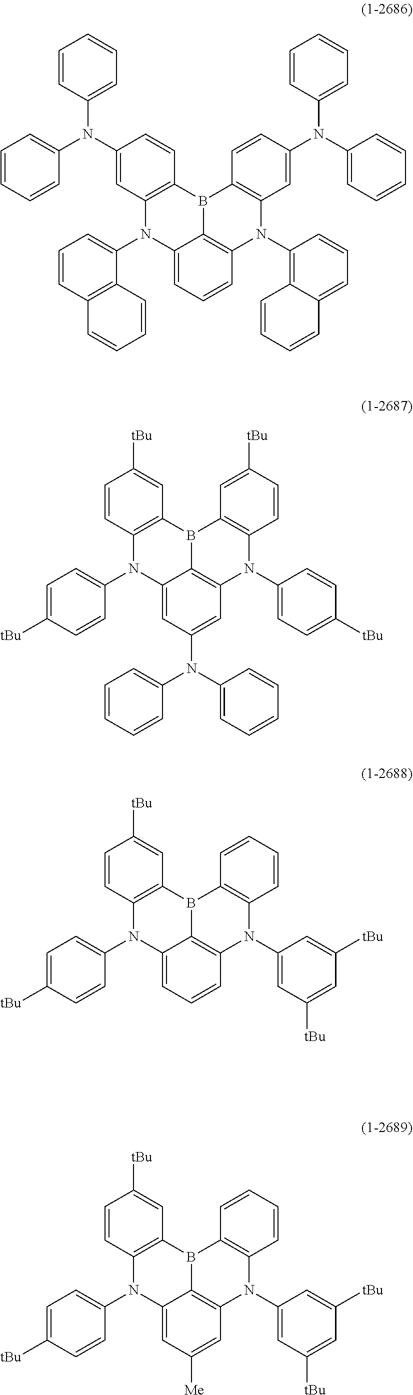 [SMILES: c1ccc(N(c2ccccc2)c2ccc3c(c2)N(c2cccc4ccccc24)c2cccc4c2B3c2ccc(N(c3ccccc3)c3ccccc3)cc2N4c2cccc3ccccc23)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4ccccc4N(c4cc(C(C)(C)C)cc(C(C)(C)C)c4)c4cccc2c43)cc1 ; Cc1cc2c3c(c1)N(c1ccc(C(C)(C)C)cc1)c1ccc(C(C)(C)C)cc1B3c1ccccc1N2c1cc(C(C)(C)C)cc(C(C)(C)C)c1]

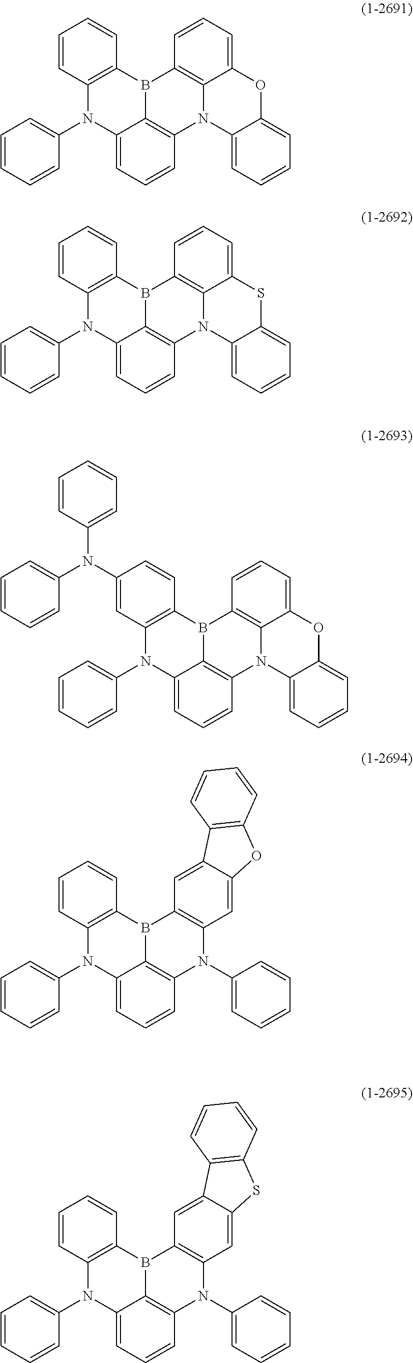

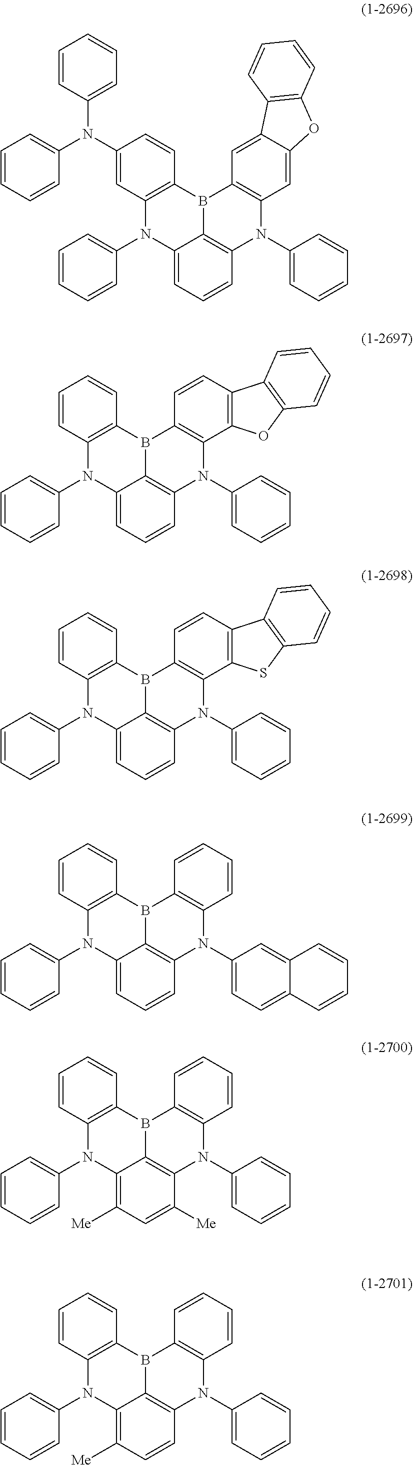 [SMILES: c1ccc(N(c2ccccc2)c2ccc3c(c2)N(c2ccccc2)c2cccc4c2B3c2cc3c(cc2N4c2ccccc2)oc2ccccc23)cc1 ; c1ccc(N2c3ccccc3B3c4ccc5c(oc6ccccc65)c4N(c4ccccc4)c4cccc2c43)cc1 ; c1ccc(N2c3ccccc3B3c4ccc5c(sc6ccccc65)c4N(c4ccccc4)c4cccc2c43)cc1 ; c1ccc(N2c3ccccc3B3c4ccccc4N(c4ccc5ccccc5c4)c4cccc2c43)cc1 ; Cc1cc(C)c2c3c1N(c1ccccc1)c1ccccc1B3c1ccccc1N2c1ccccc1 ; Cc1ccc2c3c1N(c1ccccc1)c1ccccc1B3c1ccccc1N2c1ccccc1]

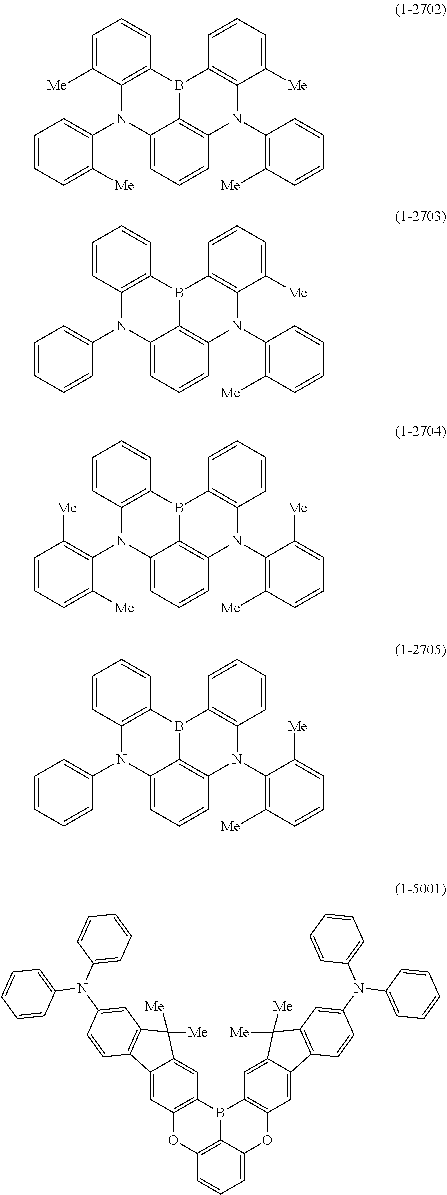

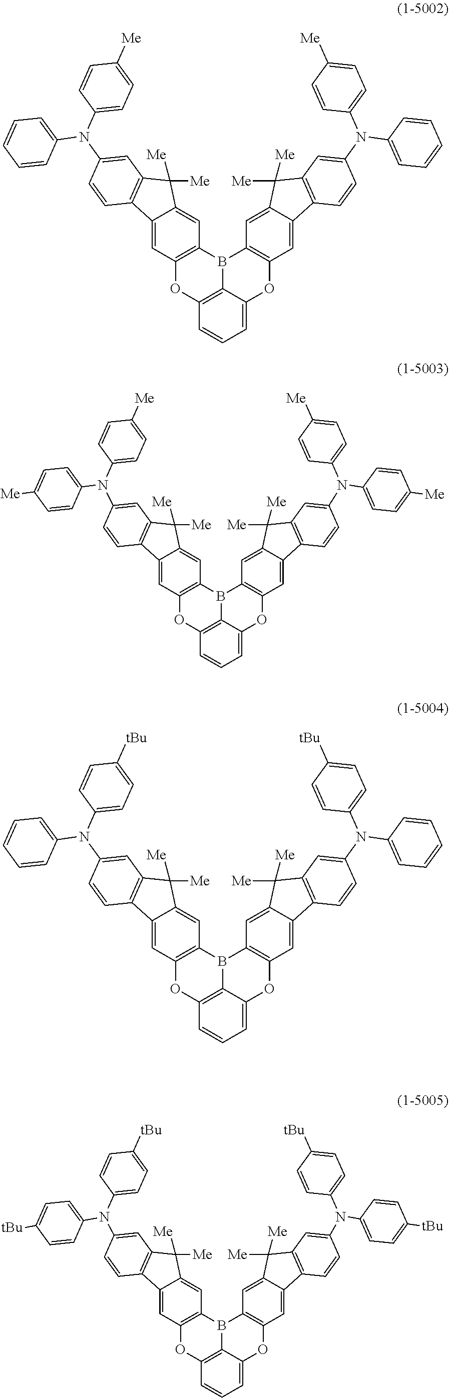

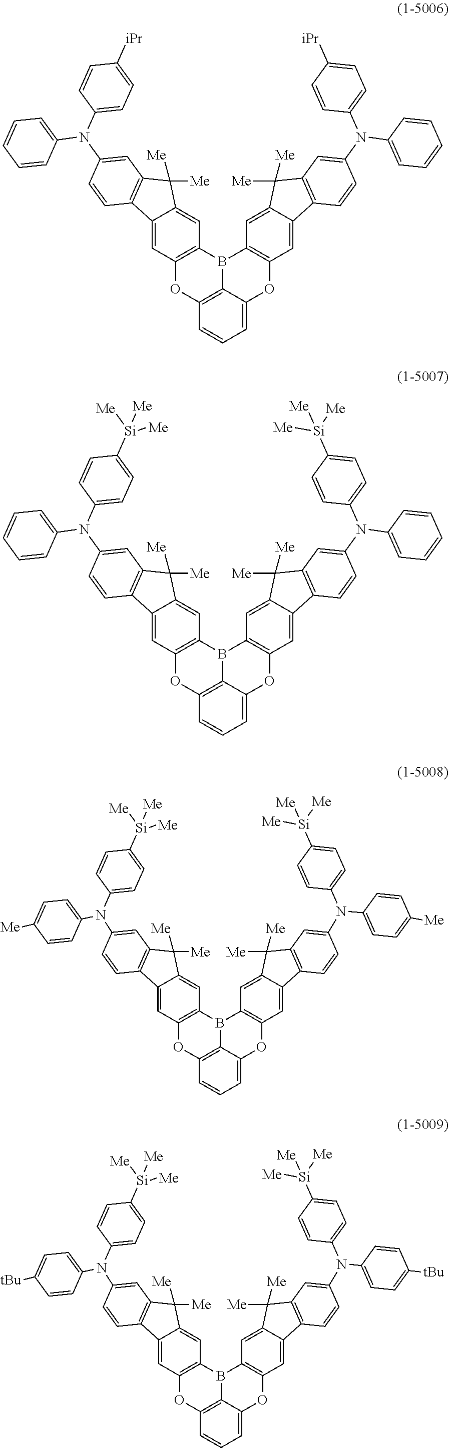

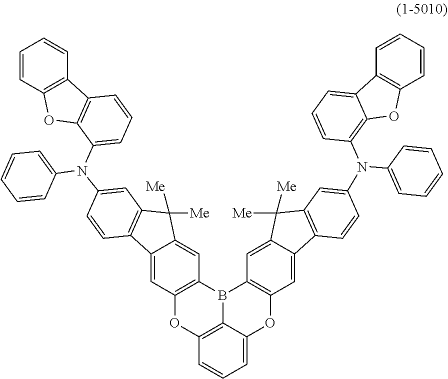 [SMILES: CC1(C)c2cc(N(c3ccccc3)c3cccc4c3oc3ccccc34)ccc2-c2cc3c(cc21)B1c2cc4c(cc2Oc2cccc(c21)O3)-c1ccc(N(c2ccccc2)c2cccc3c2oc2ccccc23)cc1C4(C)C]

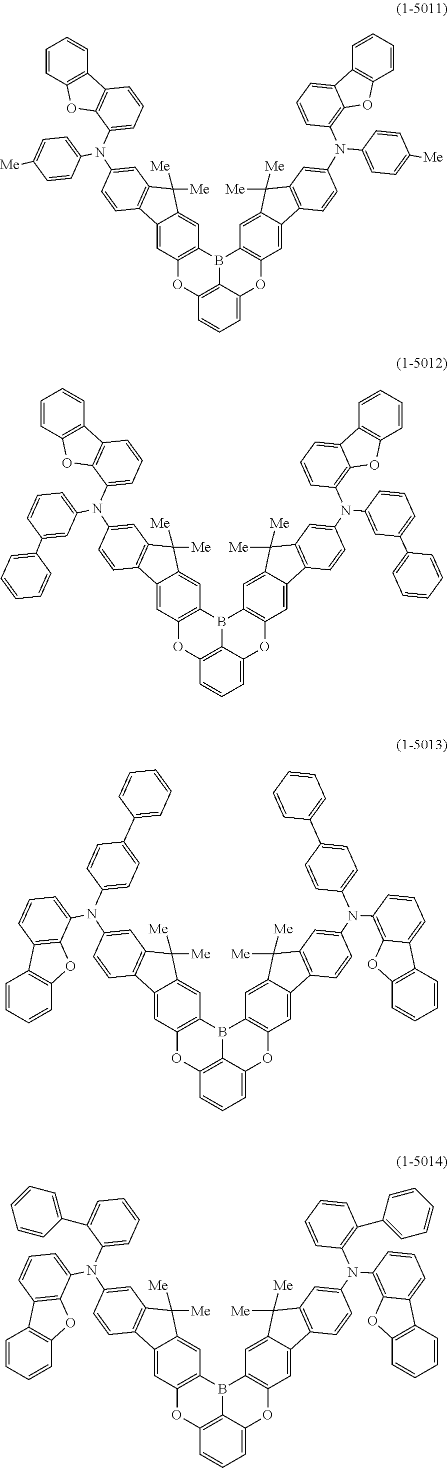

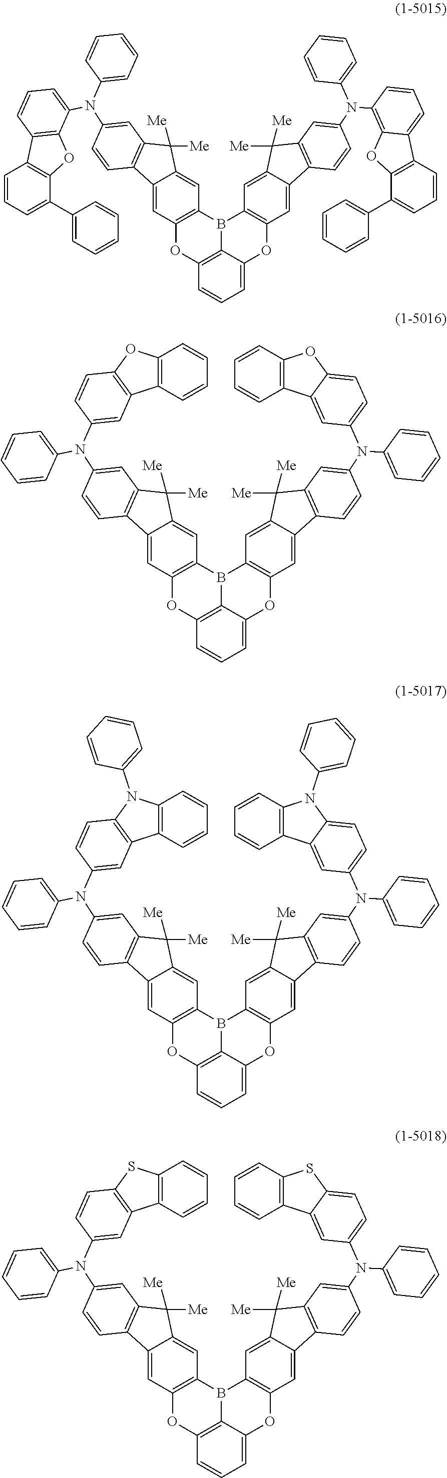

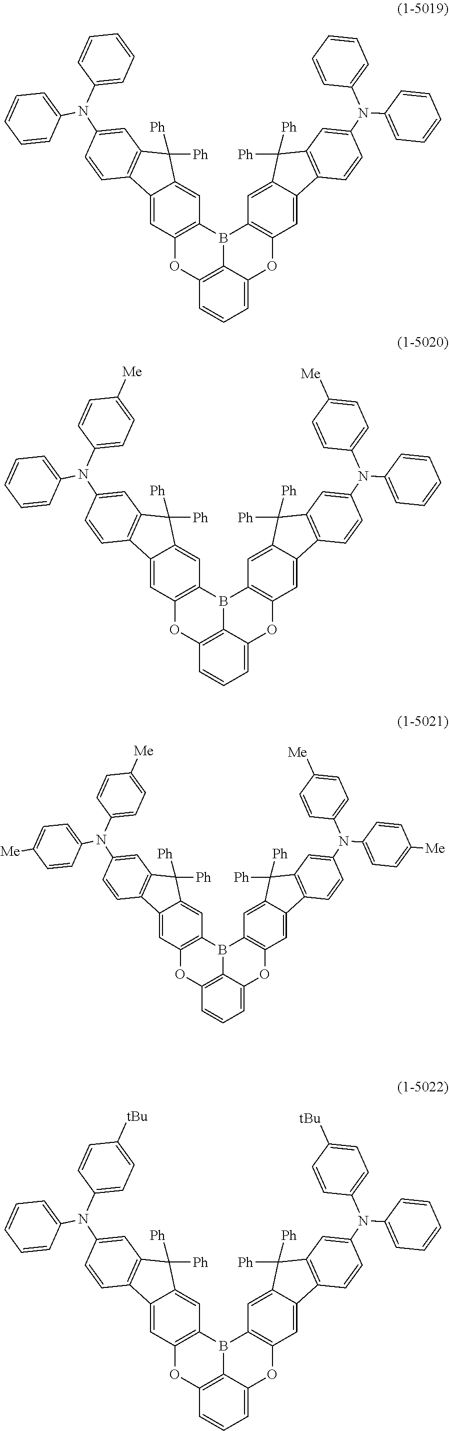

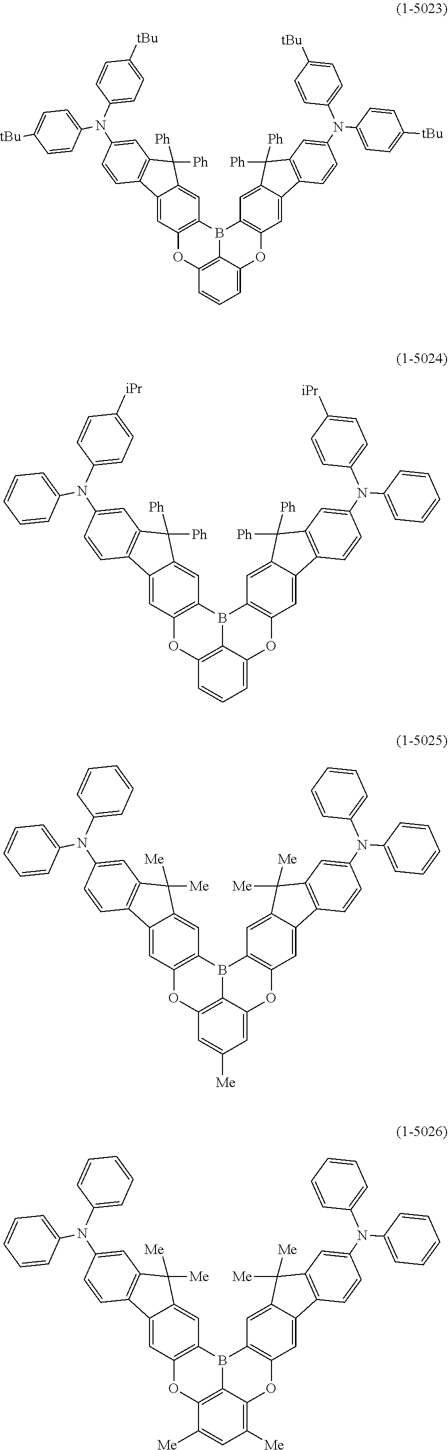

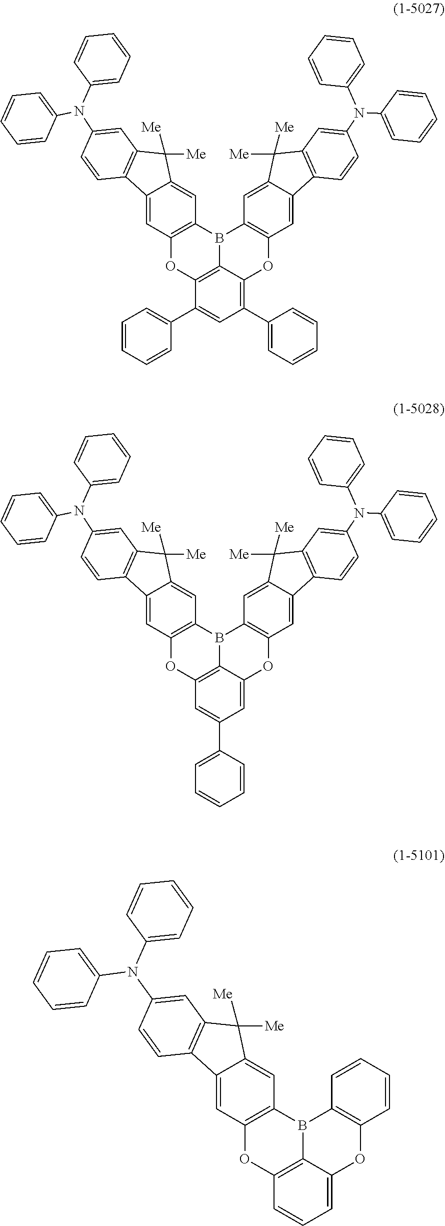 [SMILES: CC1(C)c2cc(N(c3ccccc3)c3ccccc3)ccc2-c2cc3c(cc21)B1c2cc4c(cc2Oc2c(-c5ccccc5)cc(-c5ccccc5)c(c21)O3)-c1ccc(N(c2ccccc2)c2ccccc2)cc1C4(C)C ; CC1(C)c2cc(N(c3ccccc3)c3ccccc3)ccc2-c2cc3c(cc21)B1c2cc4c(cc2Oc2cc(-c5ccccc5)cc(c21)O3)-c1ccc(N(c2ccccc2)c2ccccc2)cc1C4(C)C ; CC1(C)c2cc(N(c3ccccc3)c3ccccc3)ccc2-c2cc3c(cc21)B1c2ccccc2Oc2cccc(c21)O3]

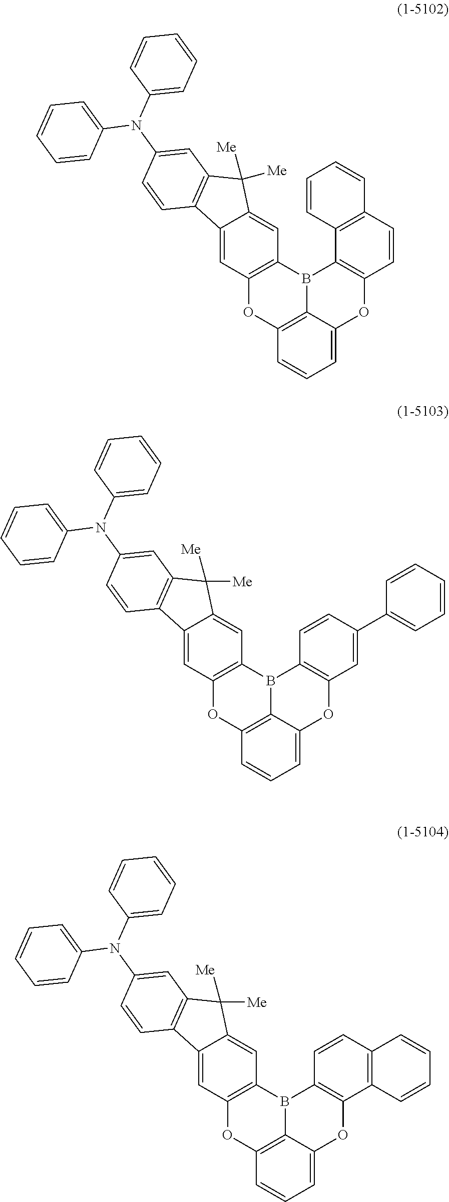 [SMILES: CC1(C)c2cc(N(c3ccccc3)c3ccccc3)ccc2-c2cc3c(cc21)B1c2c(cccc2Oc2ccc4ccccc4c21)O3 ; CC1(C)c2cc(N(c3ccccc3)c3ccccc3)ccc2-c2cc3c(cc21)B1c2ccc(-c4ccccc4)cc2Oc2cccc(c21)O3 ; CC1(C)c2cc(N(c3ccccc3)c3ccccc3)ccc2-c2cc3c(cc21)B1c2ccc4ccccc4c2Oc2cccc(c21)O3]

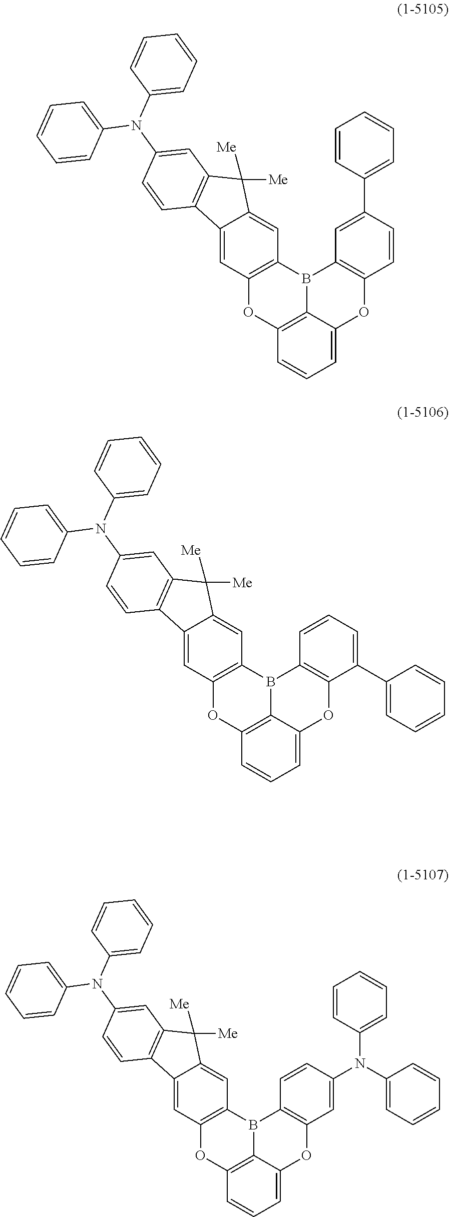 [SMILES: CC1(C)c2cc(N(c3ccccc3)c3ccccc3)ccc2-c2cc3c(cc21)B1c2cc(-c4ccccc4)ccc2Oc2cccc(c21)O3 ; CC1(C)c2cc(N(c3ccccc3)c3ccccc3)ccc2-c2cc3c(cc21)B1c2cccc(-c4ccccc4)c2Oc2cccc(c21)O3 ; CC1(C)c2cc(N(c3ccccc3)c3ccccc3)ccc2-c2cc3c(cc21)B1c2ccc(N(c4ccccc4)c4ccccc4)cc2Oc2cccc(c21)O3]

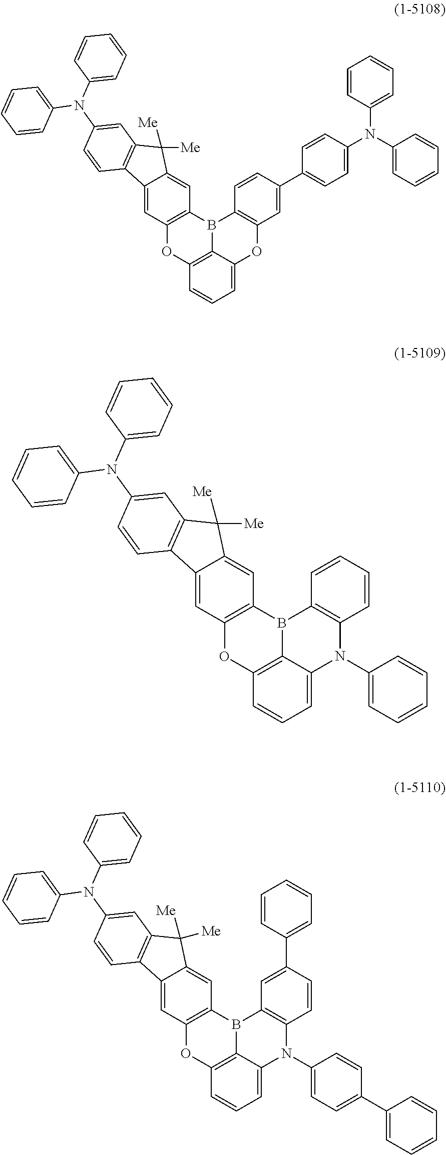 [SMILES: CC1(C)c2cc(N(c3ccccc3)c3ccccc3)ccc2-c2cc3c(cc21)B1c2ccc(-c4ccc(N(c5ccccc5)c5ccccc5)cc4)cc2Oc2cccc(c21)O3 ; CC1(C)c2cc(N(c3ccccc3)c3ccccc3)ccc2-c2cc3c(cc21)B1c2ccccc2N(c2ccccc2)c2cccc(c21)O3 ; CC1(C)c2cc(N(c3ccccc3)c3ccccc3)ccc2-c2cc3c(cc21)B1c2cc(-c4ccccc4)ccc2N(c2ccc(-c4ccccc4)cc2)c2cccc(c21)O3]

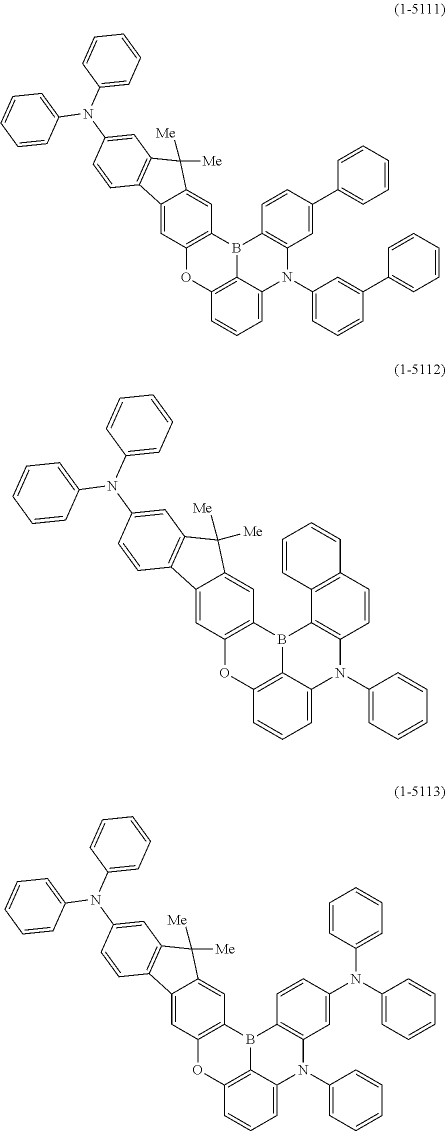 [SMILES: CC1(C)c2cc(N(c3ccccc3)c3ccccc3)ccc2-c2cc3c(cc21)B1c2ccc(-c4ccccc4)cc2N(c2cccc(-c4ccccc4)c2)c2cccc(c21)O3 ; CC1(C)c2cc(N(c3ccccc3)c3ccccc3)ccc2-c2cc3c(cc21)B1c2c(cccc2N(c2ccccc2)c2ccc4ccccc4c21)O3 ; CC1(C)c2cc(N(c3ccccc3)c3ccccc3)ccc2-c2cc3c(cc21)B1c2ccc(N(c4ccccc4)c4ccccc4)cc2N(c2ccccc2)c2cccc(c21)O3]

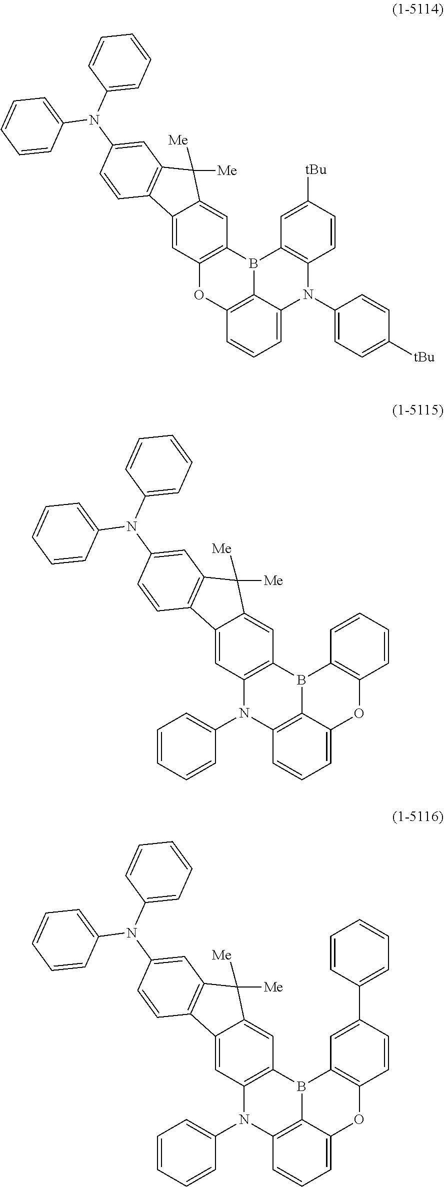 [SMILES: CC(C)(C)c1ccc(N2c3ccc(C(C)(C)C)cc3B3c4cc5c(cc4Oc4cccc2c43)-c2ccc(N(c3ccccc3)c3ccccc3)cc2C5(C)C)cc1 ; CC1(C)c2cc(N(c3ccccc3)c3ccccc3)ccc2-c2cc3c(cc21)B1c2ccccc2Oc2cccc(c21)N3c1ccccc1 ; CC1(C)c2cc(N(c3ccccc3)c3ccccc3)ccc2-c2cc3c(cc21)B1c2cc(-c4ccccc4)ccc2Oc2cccc(c21)N3c1ccccc1]

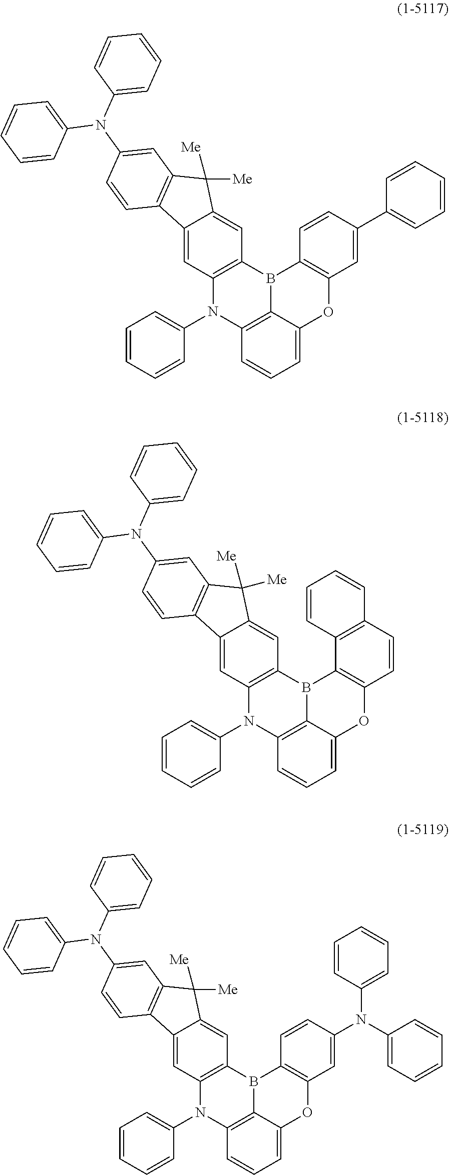 [SMILES: CC1(C)c2cc(N(c3ccccc3)c3ccccc3)ccc2-c2cc3c(cc21)B1c2ccc(-c4ccccc4)cc2Oc2cccc(c21)N3c1ccccc1 ; CC1(C)c2cc(N(c3ccccc3)c3ccccc3)ccc2-c2cc3c(cc21)B1c2c(cccc2N3c2ccccc2)Oc2ccc3ccccc3c21 ; CC1(C)c2cc(N(c3ccccc3)c3ccccc3)ccc2-c2cc3c(cc21)B1c2ccc(N(c4ccccc4)c4ccccc4)cc2Oc2cccc(c21)N3c1ccccc1]

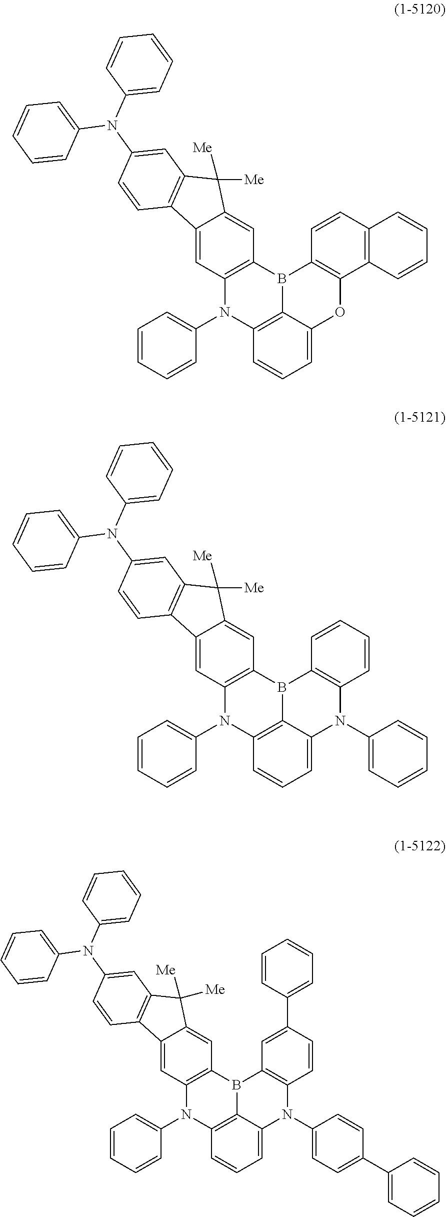 [SMILES: CC1(C)c2cc(N(c3ccccc3)c3ccccc3)ccc2-c2cc3c(cc21)B1c2ccc4ccccc4c2Oc2cccc(c21)N3c1ccccc1 ; CC1(C)c2cc(N(c3ccccc3)c3ccccc3)ccc2-c2cc3c(cc21)B1c2ccccc2N(c2ccccc2)c2cccc(c21)N3c1ccccc1 ; CC1(C)c2cc(N(c3ccccc3)c3ccccc3)ccc2-c2cc3c(cc21)B1c2cc(-c4ccccc4)ccc2N(c2ccc(-c4ccccc4)cc2)c2cccc(c21)N3c1ccccc1]

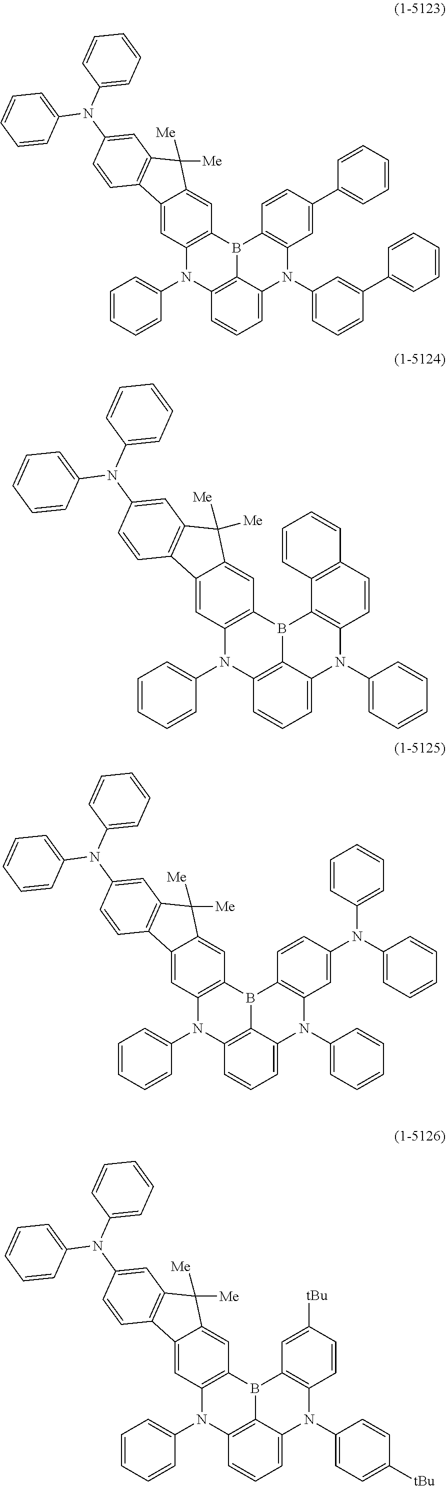 [SMILES: CC1(C)c2cc(N(c3ccccc3)c3ccccc3)ccc2-c2cc3c(cc21)B1c2ccc(-c4ccccc4)cc2N(c2cccc(-c4ccccc4)c2)c2cccc(c21)N3c1ccccc1 ; CC1(C)c2cc(N(c3ccccc3)c3ccccc3)ccc2-c2cc3c(cc21)B1c2c(cccc2N(c2ccccc2)c2ccc4ccccc4c21)N3c1ccccc1 ; CC1(C)c2cc(N(c3ccccc3)c3ccccc3)ccc2-c2cc3c(cc21)B1c2ccc(N(c4ccccc4)c4ccccc4)cc2N(c2ccccc2)c2cccc(c21)N3c1ccccc1 ; CC(C)(C)c1ccc(N2c3ccc(C(C)(C)C)cc3B3c4cc5c(cc4N(c4ccccc4)c4cccc2c43)-c2ccc(N(c3ccccc3)c3ccccc3)cc2C5(C)C)cc1]

In regard to the compound represented by formula (1) and a multimer thereof, an increase in the T1 energy (an increase by approximately 0.01 to 0.1 eV) can be expected by introducing a phenyloxy group, a carbazolyl group or a diphenylamino group into the para-position with respect to central atom “B” (boron atom) in at least one of the ring A, ring B and ring C (ring a, ring b and ring c). Particularly, when a phenyloxy group is introduced into the para-position with respect to B (boron), the HOMO on the benzene rings which are the ring A, ring B and ring C (ring a, ring b and ring c) is more localized to the meta-position with respect to the boron, while the LUMO is localized to the ortho-position and the para-position with respect to the boron. Therefore, particularly, an increase in the T1 energy can be expected.

Specific examples of such a compound include compounds represented by the following formulas (1-4501) to (1-4522).

Note that R in the formulas represents an alkyl, and may be either linear or branched. Examples thereof include a linear alkyl having 1 to 24 carbon atoms and a branched alkyl having 3 to 24 carbon atoms. An alkyl having 1 to 18 carbon atoms (branched alkyl having 3 to 18 carbon atoms) is preferable, an alkyl having 1 to 12 carbon atoms (branched alkyl having 3 to 12 carbon atoms) is more preferable, an alkyl having 1 to 6 carbon atoms (branched alkyl having 3 to 6 carbon atoms) is still more preferable, and an alkyl having 1 to 4 carbon atoms (branched alkyl having 3 to 4 carbon atoms) is particularly preferable. Other examples of R include phenyl.

Furthermore, “PhO—” represents a phenyloxy group, and this phenyl may be substituted by a linear or branched alkyl. For example, the phenyl may be substituted by a linear alkyl having 1 to 24 carbon atoms or a branched alkyl having 3 to 24 carbon atoms, an alkyl having 1 to 18 carbon atoms (a branched alkyl having 3 to 18 carbon atoms), an alkyl having 1 to 12 carbon atoms (a branched alkyl having 3 to 12 carbon atoms), an alkyl having 1 to 6 carbon atoms (a branched alkyl having 3 to 6 carbon atoms), or an alkyl having 1 to 4 carbon atoms (a branched alkyl having 3 or 4 carbon atoms).

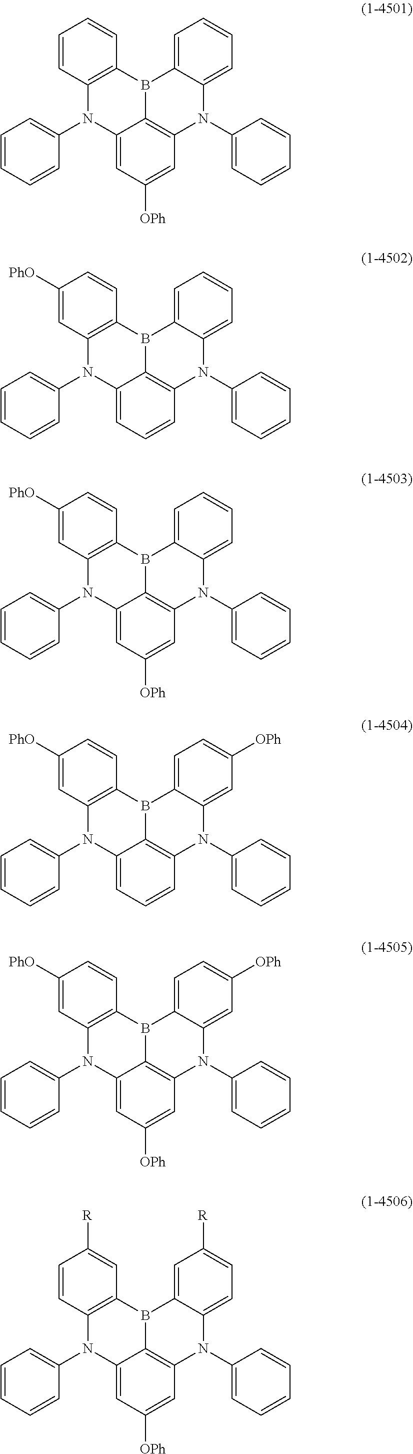

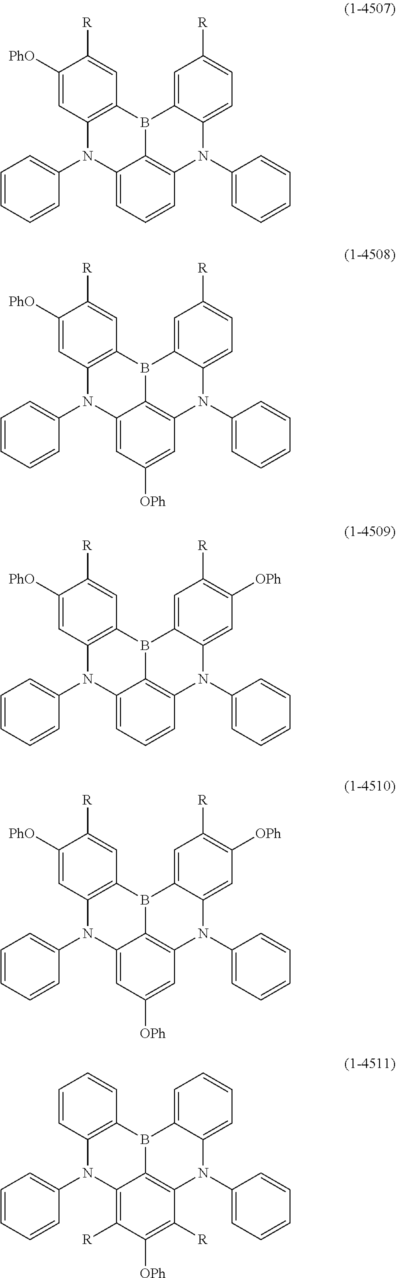

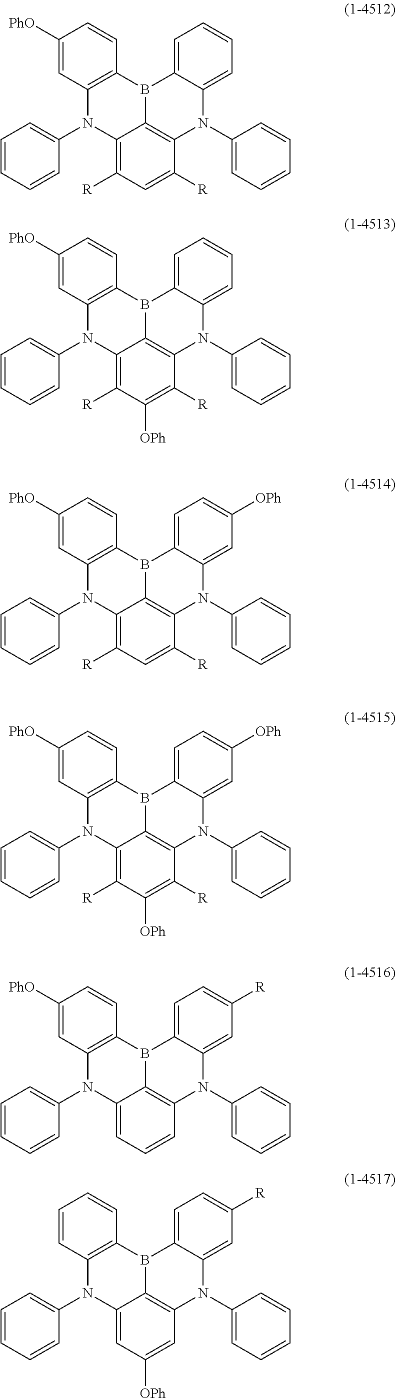

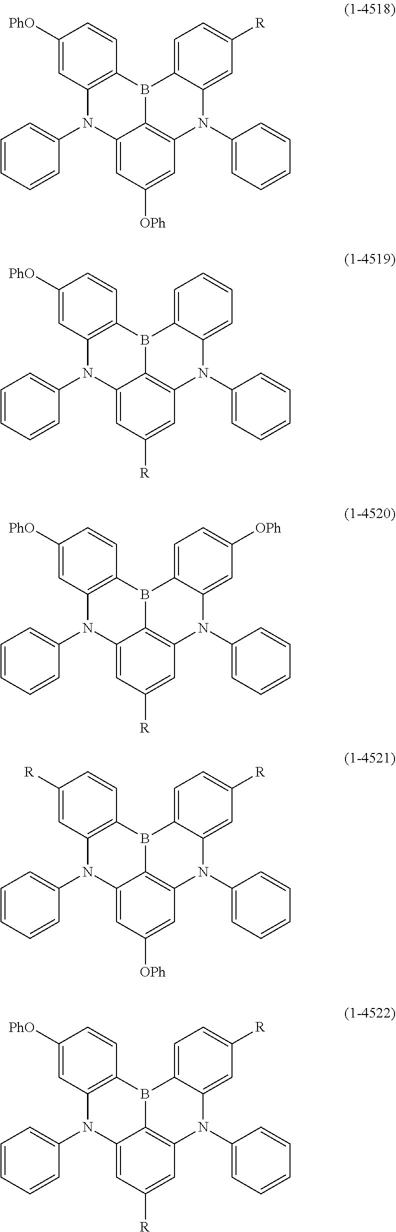

Specific examples of the compound represented by formula (1) and a multimer thereof include the above compounds in which at least one hydrogen atom in one or more aromatic rings in the compound is substituted by one or more alkyls or aryls. More preferable examples thereof include a compound substituted by 1 or 2 of alkyls each having 1 to 12 carbon atoms and aryls each having 6 to 10 carbon atoms.

Specific examples thereof include the following compounds. R's in the following formulas each independently represent an alkyl having 1 to 12 carbon atoms or an aryl having 6 to 10 carbon atoms, and preferably an alkyl or phenyl having 1 to 4 carbon atoms, and n's each independently represent 0 to 2, and preferably 1.

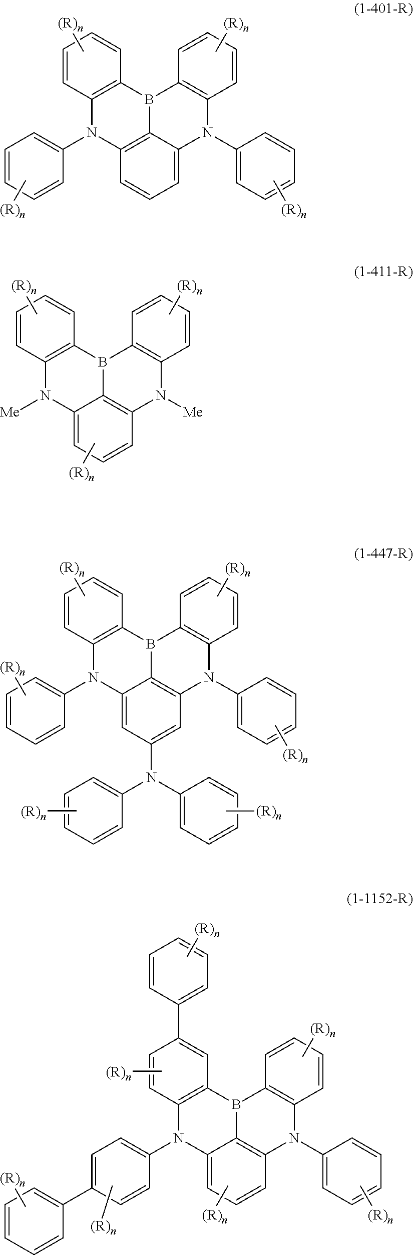

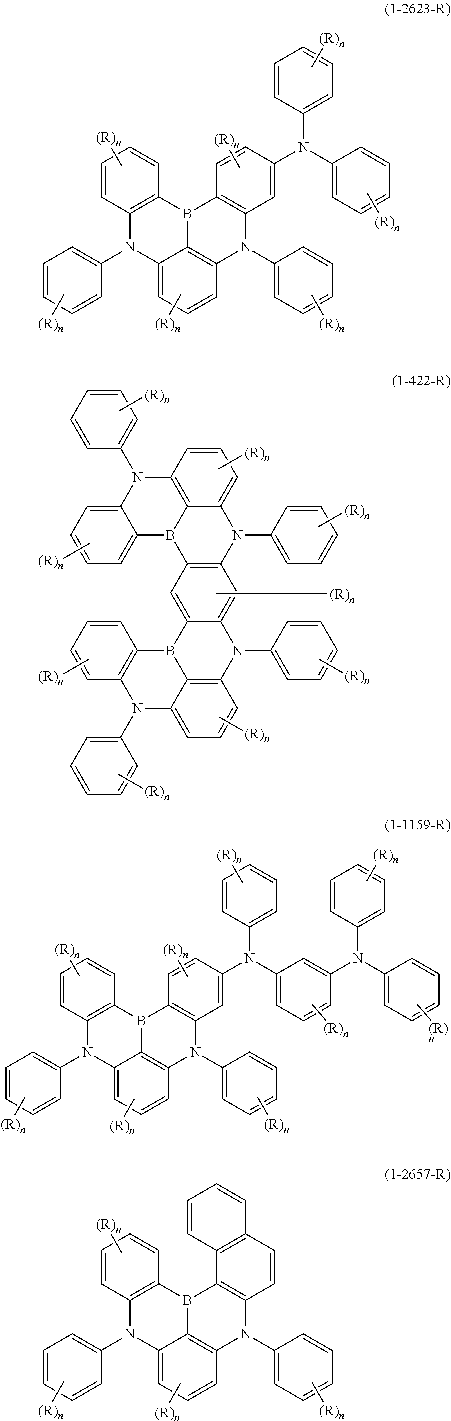

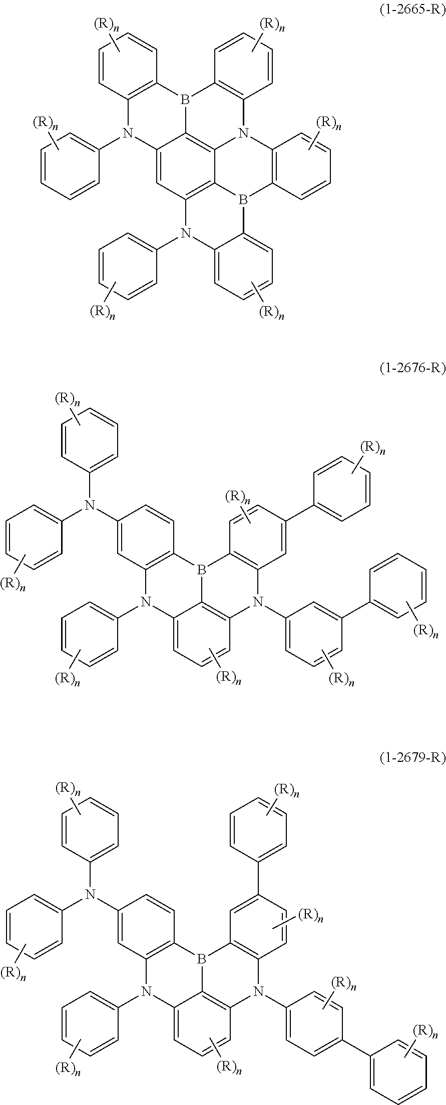

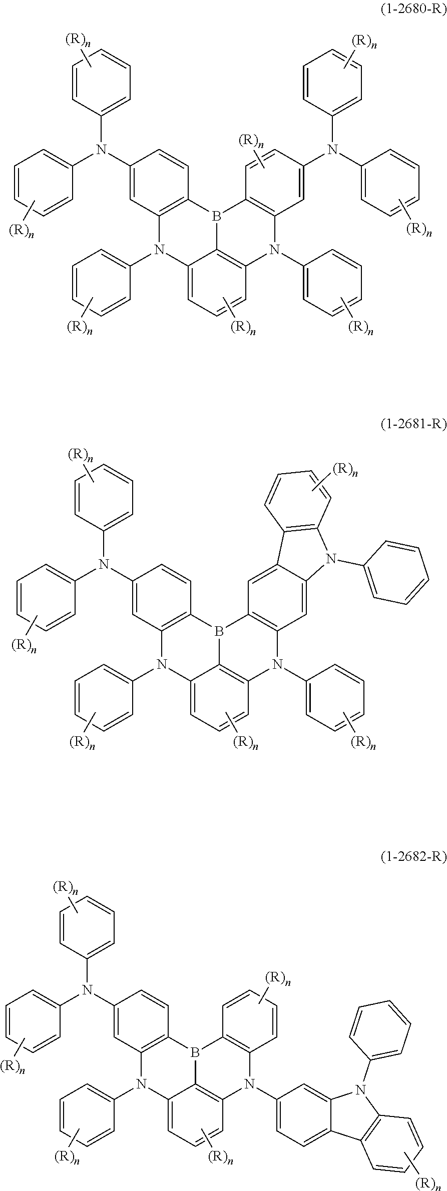

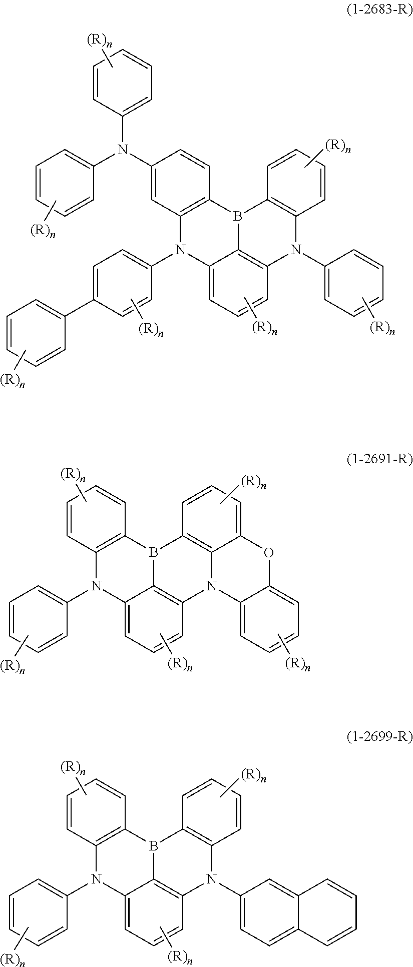

Furthermore, specific examples of the compound represented by formula (1) and a multimer thereof include a compound in which at least one hydrogen atom in one or more phenyl groups or one phenylene group in the compound is substituted by one or more alkyls each having 1 to 4 carbon atoms, and preferably one or more alkyls each having 1 to 3 carbon atoms (preferably one or more methyl groups). More preferable examples thereof include a compound in which the hydrogen atoms at the ortho-positions of one phenyl group (both of the two sites, preferably any one site) or the hydrogen atoms at the ortho-positions of one phenylene group (all of the four sites at maximum, preferably any one site) are substituted by methyl groups.

By substitution of at least one hydrogen atom at the ortho-position of a phenyl group or a p-phenylene group at a terminal in the compound by a methyl group or the like, adjacent aromatic rings are likely to intersect each other perpendicularly, and conjugation is weakened. As a result, triplet excitation energy (E_{T}) can be increased.

1-2. Method for Manufacturing a Compound Represented by Formula (1) and Multimer Thereof

In regard to the compound represented by general formula (1) or (1′) and a multimer thereof, basically, an intermediate is manufactured by first bonding the ring A (ring a), ring B (ring b) and ring C (ring c) with bonding groups (groups containing X¹ or X²) (first reaction), and then a final product can be manufactured by bonding the ring A (ring a), ring B (ring b) and ring C (ring c) with bonding groups (groups containing a central atom “B” (boron)) (second reaction). In the first reaction, a general reaction such as a Buchwald-Hartwig reaction can be utilized in a case of an amination reaction. In the second reaction, a Tandem Hetero-Friedel-Crafts reaction (continuous aromatic electrophilic substitution reaction, the same hereinafter) can be utilized. In addition, in the schemes (1) to (13) described later, although the case of “N—R” as X¹ and X² is described, the same applies to the case of “O”. Note that each code in the structural formulas the following schemes (1) to (13) are defined in the same manner as those in formulas (1) and (1′).

As illustrated in the following schemes (1) and (2), the second reaction is a reaction for introducing a central atom “B” (boron) which bonds the ring A (ring a), ring B (ring b) and ring C (ring c). First, a hydrogen atom between X¹ and X² (>N—R) is ortho-metalated with n-butyllithium, sec-butyllithium, t-butyllithium, or the like. Subsequently, boron trichloride, boron tribromide, or the like is added thereto to perform lithium-boron metal exchange, and then a Brønsted base such as N,N-diisopropylethylamine is added thereto to induce a Tandem Bora-Friedel-Crafts reaction. Thus, a desired product can be obtained. In the second reaction, a Lewis acid such as aluminum trichloride may be added in order to accelerate the reaction.

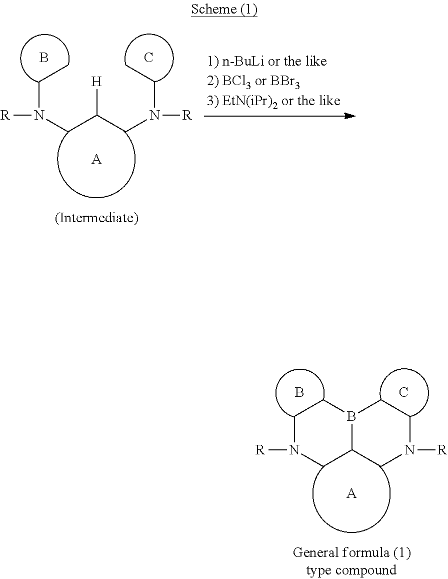

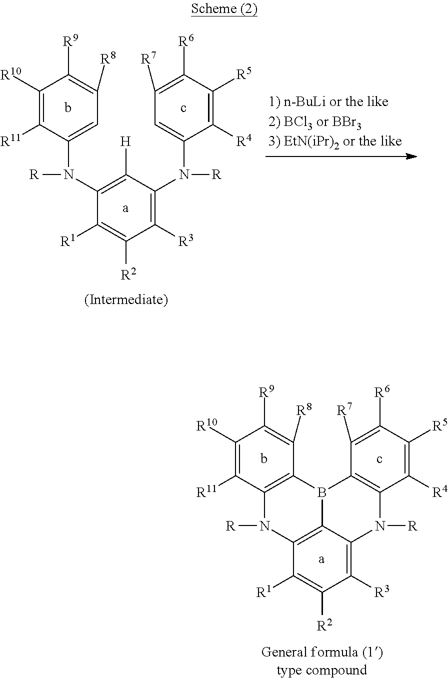

Incidentally, the scheme (1) or (2) mainly illustrates a method for manufacturing a compound represented by general formula (1) or (1′). However, a multimer thereof can be manufactured using an intermediate having a plurality of ring A's (ring a's), ring B's (ring b's) and ring C's (ring c's). More specifically, the manufacturing method will be described by the following schemes (3) to (5). In this case, a desired product may be obtained by increasing the amount of the reagent used therein such as butyllithium to a double amount or a triple amount.

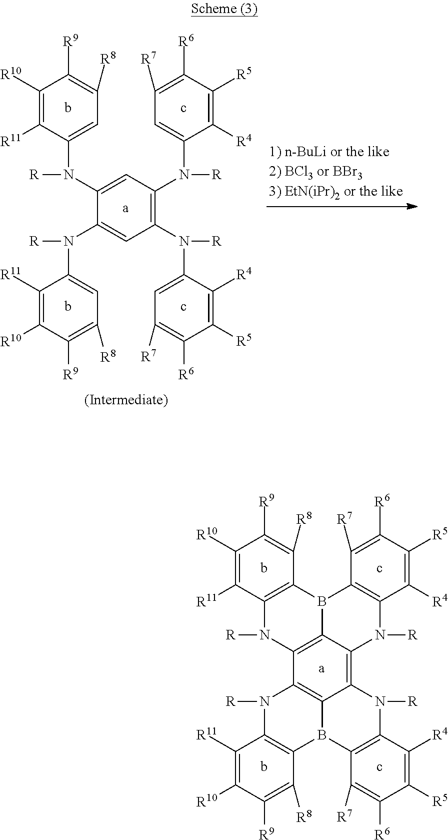

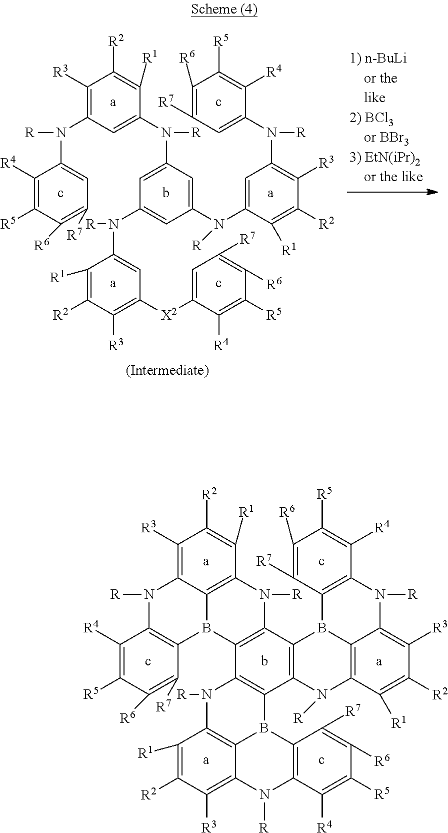

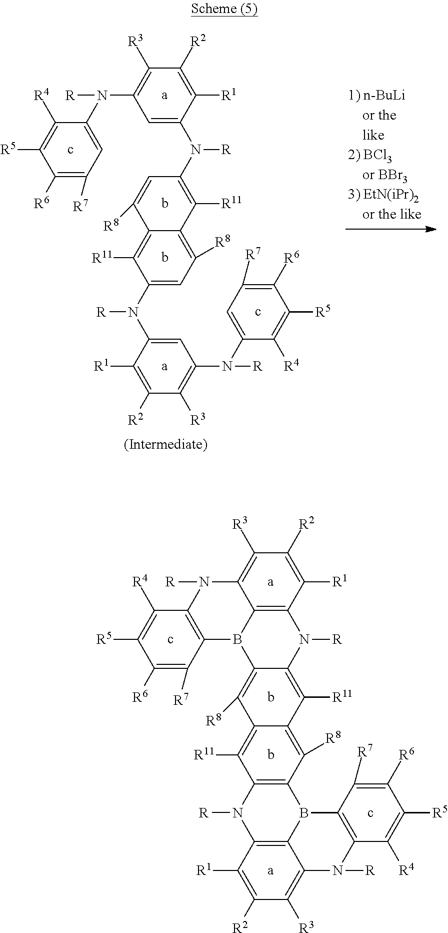

In the above schemes, lithium is introduced into a desired position by ortho-metalation. However, lithium can also be introduced into a desired position by halogen-metal exchange by introducing a bromine atom or the like to a position to which it is wished to introduce lithium, as in the following schemes (6) and (7).

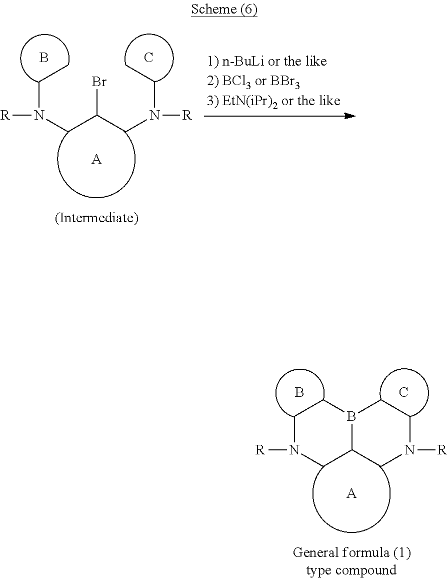

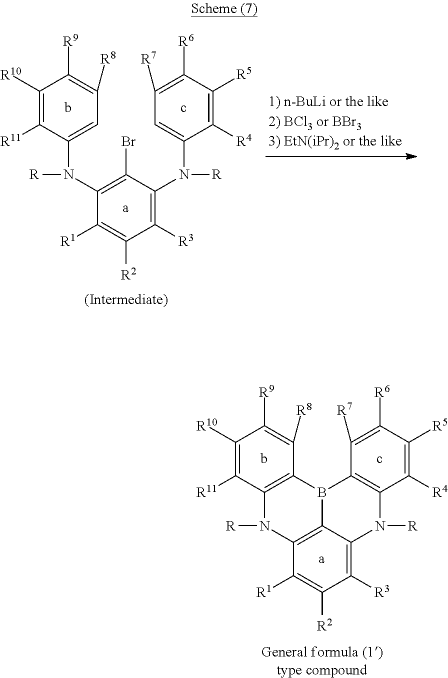

Furthermore, also in regard to the method for manufacturing a multimer described in scheme (3), a lithium atom can be introduced to a desired position also by halogen-metal exchange by introducing a halogen atom such as a bromine atom or a chlorine atom to a position to which it is wished to introduce a lithium atom, as in the above schemes (6) and (7) (the following schemes (8), (9), and (10)).

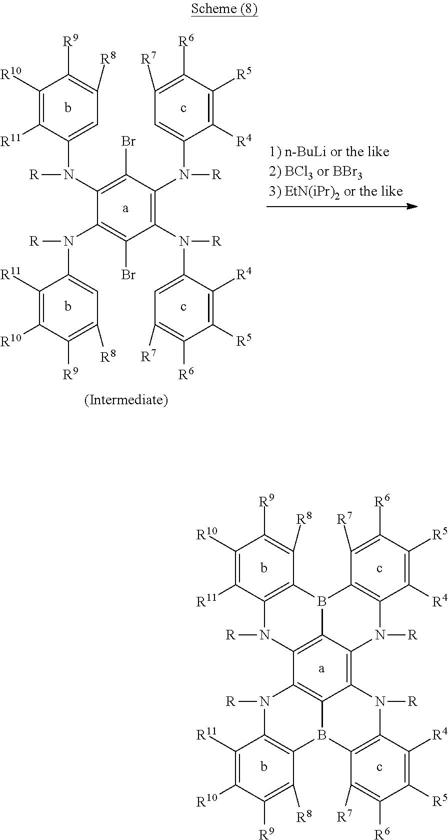

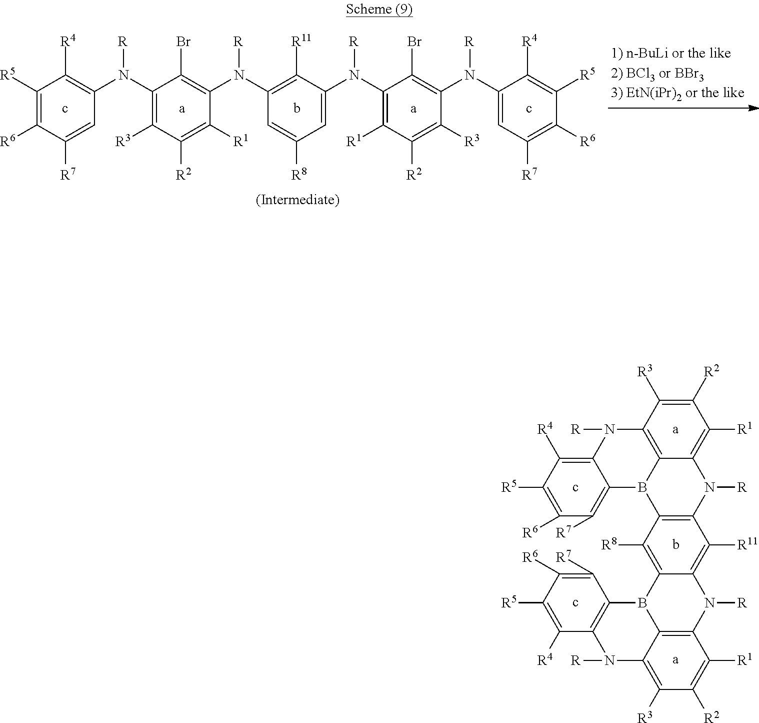

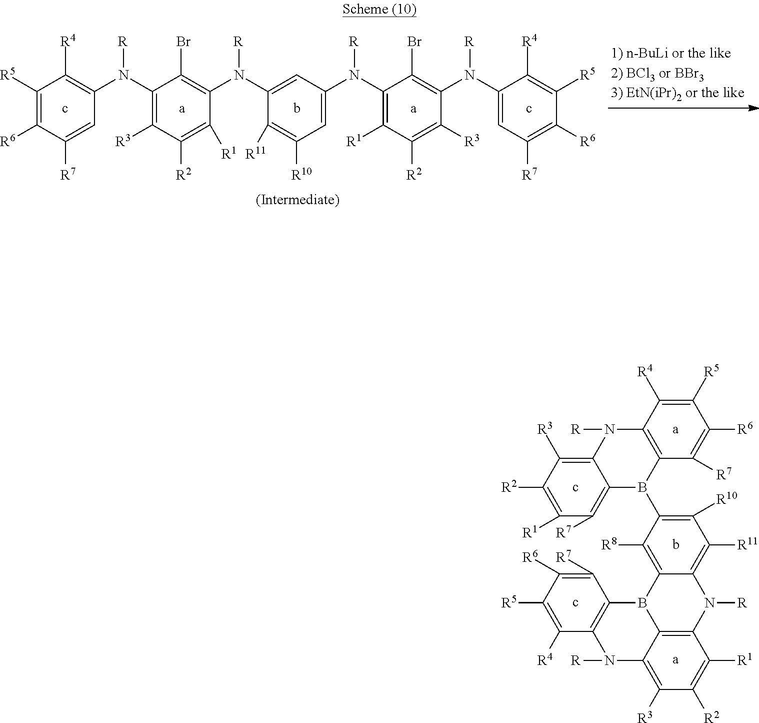

According to this method, a desired product can also be synthesized even in a case in which ortho-metalation cannot be achieved due to the influence of substituents, and therefore the method is useful.

Specific examples of the solvent used in the above reactions include t-butylbenzene and xylene.

By appropriately selecting the above synthesis method and appropriately selecting raw materials to be used, it is possible to synthesize a compound having a substituent at a desired position and a multimer thereof.

Furthermore, in general formula (1′), adjacent groups among the substituents R¹ to R¹¹ of the ring a, ring b and ring c may be bonded to each other to form an aryl ring or a heteroaryl ring together with the ring a, ring b or ring c, and at least one hydrogen atom in the ring thus formed may be substituted by an aryl or a heteroaryl. Therefore, in a compound represented by general formula (1′), a ring structure constituting the compound changes as represented by formulas (1′-1) and (1′-2) of the following schemes (11) and (12) according to a mutual bonding form of substituents in the ring a, ring b, and ring c. These compounds can be synthesized by applying synthesis methods illustrated in the above schemes (1) to (10) to intermediates illustrated in the following schemes (11) and (12).

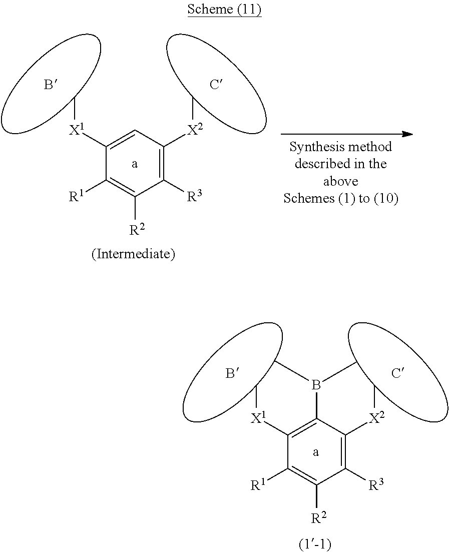

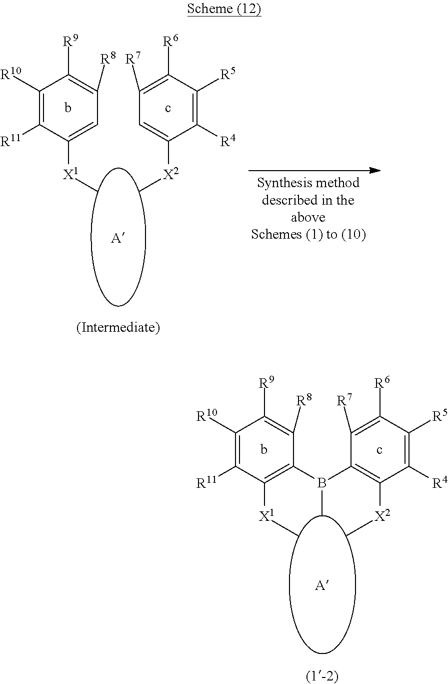

Ring A′, ring B′ and ring C′ in the above formulas (1′-1) and (1′-2) each represent an aryl ring or a heteroaryl ring formed by bonding adjacent groups among the substituents R¹ to R¹¹ together with the ring a, ring b, and ring c, respectively (may also be a fused ring obtained by fusing another ring structure to the ring a, ring b, or ring c). Incidentally, although not indicated in the formula, there is also a compound in which all of the ring a, ring b, and ring c have been changed to the ring A′, ring B′ and ring C′.

Furthermore, the provision that “R of the N—R is bonded to the ring a, ring b, and/or ring c with —O—, —S—, —C(—R)₂—, or a single bond” in general formulas (1′) can be expressed as a compound having a ring structure represented by formula (1′-3-1) of the following scheme (13), in which X¹ or X² is incorporated into the fused ring B′ or fused ring C′, or a compound having a ring structure represented by formula (1′-3-2) or (1′-3-3), in which X¹ or X² is incorporated into the fused ring A′. Such a compound can be synthesized by applying the synthesis methods illustrated in the schemes (1) to (10) to the intermediate represented by the following scheme (13).

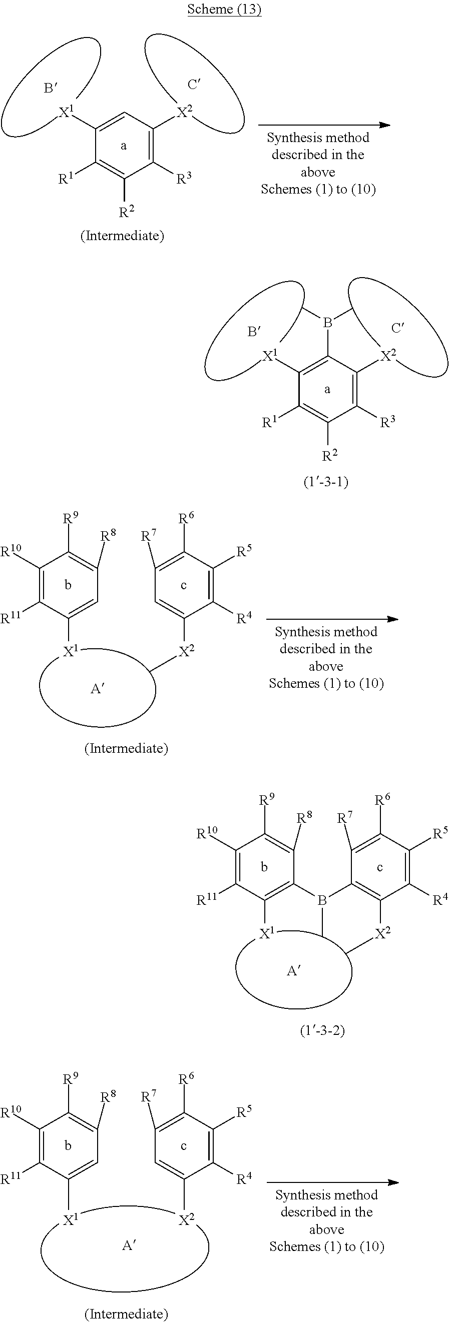

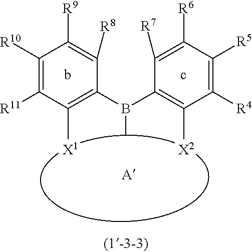

Furthermore, regarding the synthesis methods of the above schemes (1) to (13), there is shown an example of carrying out the Tandem Hetero-Friedel-Crafts reaction by ortho-metalating a hydrogen atom (or a halogen atom) between X¹ and X² with butyllithium or the like, before boron trichloride, boron tribromide or the like is added. However, the reaction may also be carried out by adding boron trichloride, boron tribromide or the like without conducting ortho-metalation using buthyllithium or the like.

Note that examples of an ortho-metalation reagent used for the above schemes (1) to (13) include an alkyllithium such as methyllithium, n-butyllithium, sec-butyllithium, or t-butyllithium; and an organic alkali compound such as lithium diisopropylamide, lithium tetramethylpiperidide, lithium hexamethyldisilazide, or potassium hexamethyldisilazide.

Incidentally, examples of a metal exchanging reagent for metal-“B” (boron) used for the above schemes (1) to (13) include a halide of boron such as trifluoride of boron, trichloride of boron, tribromide of boron, or triiodide of boron; an aminated halide of boron such as CIPN(NEt₂)₂; an alkoxylation product of boron; and an aryloxylation product of boron.

Incidentally, examples of the Brønsted base used for the above schemes (1) to (13) include N,N-diisopropylethylamine, triethylamine, 2,2,6,6-tetramethylpiperidine, 1,2,2,6,6-pentamethylpiperidine, N,N-dimethylaniline, N,N-dimethyltoluidine, 2,6-lutidine, sodium tetraphenylborate, potassium tetraphenylborate, triphenylborane, tetraphenylsilane, Ar₄BNa, Ar₄BK, Ar₃B, and Ar₄Si (Ar represents an aryl such as phenyl).

Examples of a Lewis acid used for the above schemes (1) to (13) include AlCl₃, AlBr₃, AlF₃, BF₃.OEt₂, BC₁₃, BBr₃, GaCl₃, GaBr₃, InCl₃, InBr₃, In(OTf)₃, SnCl₄, SnBr₄, AgOTf, ScCl₃, Sc(OTf)₃, ZnCl₂, ZnBr₂, Zn(OTf)₂, MgCl₂, MgBr₂, Mg(OTf)₂, LiOTf, NaOTf, KOTf, Me₃SiOTf, Cu(OTf)₂, CuCl₂, YCl₃, Y(OTf)₃, TiCl₄, TiBr₄, ZrCl₄, ZrBr₄, FeCl₃, FeBr₃, CoCl₃, and CoBr₃.

In the above schemes (1) to (13), a Brønsted base or a Lewis acid may be used in order to accelerate the Tandem Hetero Friedel-Crafts reaction. However, in a case where a halide of boron such as trifluoride of boron, trichloride of boron, tribromide of boron, or triiodide of boron is used, an acid such as hydrogen fluoride, hydrogen chloride, hydrogen bromide, or hydrogen iodide is generated along with progress of an aromatic electrophilic substitution reaction. Therefore, it is effective to use a Brønsted base that captures an acid. On the other hand, in a case where an aminated halide of boron or an alkoxylation product of boron is used, an amine or an alcohol is generated along with progress of the aromatic electrophilic substitution reaction. Therefore, in many cases, it is not necessary to use a Brønsted base. However, leaving ability of an amino group or an alkoxy group is low, and therefore it is effective to use a Lewis acid that promotes leaving of these groups.

A compound represented by formula (1) or a multimer thereof also includes compounds in which at least a portion of hydrogen atoms are substituted by deuterium atoms or substituted by cyanos or halogen atoms such as fluorine atoms or chlorine atoms. However, these compounds can be synthesized as described above using raw materials that are deuterated, fluorinated, chlorinated or cyanated at desired sites.

1-3. Compound Represented by General Formula (2)

The compound represented by general formula (2) basically functions as a host material.

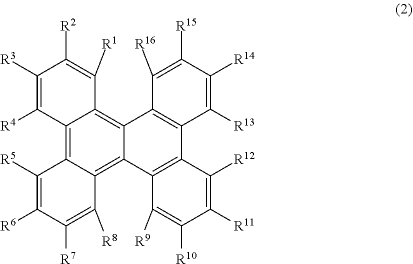

In the above formula (2),

R¹ to R¹⁶ each independently represent a hydrogen atom, an aryl, a heteroaryl (the heteroaryl may be bonded to the dibenzochrysene skeleton in the above formula (2) via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, adjacent groups among R¹ to R¹⁶ may be bonded to each other to form a fused ring, and at least one hydrogen atom in the formed ring may be substituted by an aryl, a heteroaryl (the heteroaryl may be bonded to the formed ring via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, and

at least one hydrogen atom in the compound represented by formula (2) may be substituted by a halogen atom, a cyano, or a deuterium atom.

Examples of the “aryl” R¹ to R¹⁶ include an aryl having 6 to 30 carbon atoms. An aryl having 6 to 16 carbon atoms is preferable, an aryl having 6 to 14 carbon atoms is more preferable, an aryl having 6 to 12 carbon atoms is still more preferable, and an aryl having 6 to 10 carbon atoms is particularly preferable.

Specific examples of the aryl include phenyl which is a monocyclic system; biphenylyl which is a bicyclic system; naphthyl which is a fused bicyclic system; terphenylyl (m-terphenylyl, o-terphenylyl, or p-terphenylyl) which is a tricyclic system; anthracenyl, acenaphthylenyl, fluorenyl, phenalenyl, and phenanthrenyl which are fused tricyclic systems; triphenylenyl, and naphthacenyl which are fused tetracyclic systems; and perylenyl and pentacenyl which are fused pentacyclic systems.

Examples of the “heteroaryl” in R¹ to R¹⁶ include a heteroaryl having 2 to 30 carbon atoms. A heteroaryl having 2 to 25 carbon atoms is preferable, a heteroaryl having 2 to 20 carbon atoms is more preferable, a heteroaryl having 2 to 15 carbon atoms is still more preferable, and a heteroaryl having 2 to 10 carbon atoms is particularly preferable. In addition, examples of the heteroaryl include a heterocyclic ring containing 1 to 5 heteroatoms, selected from an oxygen atom, a sulfur atom, and a nitrogen atom in addition to a carbon atom as a ring-constituting atom.

Specific examples of the heteroaryl include a pyrrolyl, an oxazolyl, an isoxazolyl, a thiazolyl, an isothiazolyl, an imidazolyl, an oxadiazolyl, a thiadiazolyl, a triazolyl, a tetrazolyl, a pyrazolyl, a pyridyl, a pyrimidinyl, a pyridazinyl, a pyrazinyl, a triazinyl, an indolyl, an isoindolyl, a 1H-indazolyl, a benzoimidazolyl, a benzoxazolyl, a benzothiazolyl, a 1H-benzotriazolyl, a quinolyl, an isoquinolyl, a cinnolyl, a quinazolyl, a quinoxalinyl, a phthalazinyl, a naphthyridinyl, a purinyl, a pteridinyl, a carbazolyl, an acridinyl, a phenoxathiinyl, a phenoxazinyl, a phenothiazinyl, a phenazinyl, an indolizinyl, a furyl, a benzofuranyl, an isobenzofuranyl, a dibenzofuranyl, a thienyl, a benzo[b]thienyl, a dibenzothienyl, a furazanyl, an oxadiazolyl, a thianthrenyl, a naphthobenzofuranyl, and a naphthobenzothienyl.

Specific examples of the heteroaryl include a monovalent group having a structure of the following formula (2-Ar1), (2-Ar2), (2-Ar3), (2-Ar4), or (2-Ar5).

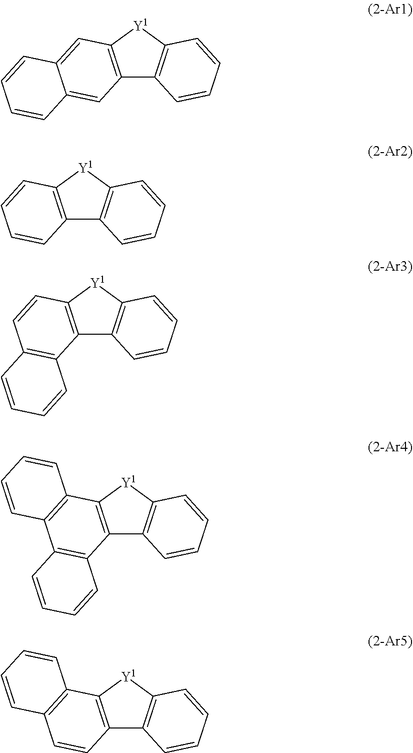

In formulas (2-Ar1) to (2-Ar5), Y¹'s each independently represent O, S, or N—R, and R represents a phenyl, a biphenylyl, a naphthyl, an anthracenyl, or a hydrogen atom, and

at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.

The heteroaryl may be bonded to a dibenzochrysene skeleton in the above formula (2) via a linking group. That is, it may be possible not only that the dibenzochrysene skeleton in formula (2) and the heteroaryl are directly bonded to each other, but also that the dibenzochrysene skeleton in formula (2) and the heteroaryl are bonded to each other via a linking group therebetween. Examples of the linking group include a phenylene, a biphenylene, a naphthylene, an anthracenylene, a methylene, an ethylene, —OCH₂CH₂—, —CH₂CH₂O—, and —OCH₂CH₂O—.

The “diarylamino”, “diheteroarylamino”, and “arylheteroarylamino” in R¹ to R¹⁶ are groups in which an amino group is substituted by two aryl groups, two heteroaryl groups, and one aryl group and one heteroaryl group, respectively. For the aryl and the heteroaryl herein, the above description of the “aryl” and “heteroaryl” can be cited.

The “alkyl” in R¹ to R¹⁶ may be either linear or branched, and examples thereof include a linear alkyl having 1 to 30 carbon atoms and a branched alkyl having 3 to 30 carbon atoms. An alkyl having 1 to 24 carbon atoms (branched alkyl having 3 to 24 carbon atoms) is preferable, an alkyl having 1 to 18 carbon atoms (branched alkyl having 3 to 18 carbon atoms) is more preferable, an alkyl having 1 to 12 carbon atoms (branched alkyl having 3 to 12 carbon atoms) is still more preferable, an alkyl having 1 to 6 carbon atoms (branched alkyl having 3 to 6 carbon atoms) is still more preferable, an alkyl having 1 to 4 carbon atoms (branched alkyl having 3 to 4 carbon atoms) is still more preferable, and an alkyl having 1 to 3 carbon atoms (branched alkyl having 3 carbon atoms) is particularly preferable.

Specific examples of the alkyl include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl, 2-ethylbutyl, n-heptyl, l-methylhexyl, n-octyl, t-octyl, 1-methylheptyl, 2-ethylhexyl, 2-propylpentyl, n-nonyl, 2,2-dimethylheptyl, 2,6-dimethyl-4-heptyl, 3,5,5-trimethylhexyl, n-decyl, n-undecyl, 1-methyldecyl, n-dodecyl, n-tridecyl, 1-hexylheptyl, n-tetradecyl, n-pentadecyl, n-hexadecyl, n-heptadecyl, n-octadecyl, n-eicosyl, and the like.

Examples of the “alkenyl” in R¹ to R¹⁶ include an alkenyl having 2 to 30 carbons. An alkenyl having 2 to 20 carbon atoms is preferable, an alkenyl having 2 to 10 carbon atoms is more preferable, an alkenyl having 2 to 6 carbon atoms is still more preferable, and an alkenyl having 2 to 4 carbon atoms is particularly preferable.

The preferable alkenyls is a vinyl, a 1-propenyl, a 2-propenyl, a 1-butenyl, a 2-butenyl, a 3-butenyl, a 1-pentenyl, a 2-pentenyl, a 3-pentenyl, a 4-pentenyl, a 1-hexenyl, a 2-hexenyl, a 3-hexenyl, a 4-hexenyl, or a 5-hexenyl.

Examples of the “alkoxy” in R¹ to R¹⁶ include a linear alkoxy having 1 to 30 carbon atoms and a branched alkoxy having 3 to 30 carbon atoms. An alkoxy having 1 to 24 carbon atoms (branched alkoxy having 3 to 24 carbon atoms) is preferable, an alkoxy having 1 to 18 carbon atoms (branched alkoxy having 3 to 18 carbon atoms) is more preferable, an alkoxy having 1 to 12 carbon atoms (branched alkoxy having 3 to 12 carbon atoms) is still more preferable, an alkoxy having 1 to 6 carbon atoms (branched alkoxy having 3 to 6 carbon atoms) is still more preferable, and an alkoxy having 1 to 4 carbon atoms (branched alkoxy having 3 to 4 carbon atoms) is particularly preferable.

Specific examples of the alkoxy include a methoxy, an ethoxy, a propoxy, an isopropoxy, a butoxy, an isobutoxy, a s-butoxy, a t-butoxy, a pentyloxy, a hexyloxy, a heptyloxy, an octyloxy, and the like.

Examples of the “aryloxy” in R¹ to R¹⁶ include a group in which a hydrogen atom of a hydroxyl group is substituted by an aryl. For this aryl, those described as the above “aryl” can be cited.

At least one hydrogen atom in the aryl, heteroaryl, diarylamino, diheteroarylamino, arylheteroarylamino, alkyl, alkenyl, alkoxy, or aryloxy as R¹ to R¹⁶ may be substituted by an aryl, a heteroaryl, or an alkyl. For the aryl, heteroaryl, or alkyl for substitution, the above description of the “aryl”, “heteroaryl”, or “alkyl” can be cited.

Adjacent groups among R¹ to R¹⁶ in formula (2) may be bonded to each other to form a fused ring. The fused ring thus formed is a ring formed by bonding R¹ and R¹⁶, R⁴ and R⁵, R⁸ and R⁹, or R¹² and R¹³ to each other, or a ring formed by bonding groups in a combination other than these combinations and fused to the four outer benzene rings in formula (2), and is an aliphatic ring or an aromatic ring. An aromatic ring is preferable, and examples of the structure including the outer benzene rings in formula (2) include a naphthalene ring and a phenanthrene ring.

At least one hydrogen atom in the fused ring thus formed may be substituted by an aryl, a heteroaryl (the heteroaryl may be bonded to the ring thus formed via a linking group), a diarylamino, a diheteroarylamino, an arylheteroarylamino, an alkyl, an alkenyl, an alkoxy, or an aryloxy, and at least one hydrogen atom in these may be substituted by an aryl, a heteroaryl, or an alkyl. For these substituents, the above description of the “aryl”, “heteroaryl”, “diarylamino”, “diheteroarylamino”, “arylheteroarylamino”, “alkyl”, “alkenyl”, “alkoxy”, or “aryloxy” can be cited.

In the compound represented by general formula (2), R¹, R⁴, R⁵, R⁸, R⁹, R¹², R¹³, and R¹⁶ preferably each represent a hydrogen atom. In this case, R², R³, R⁶, R⁷, R¹⁰, R¹¹, R¹⁴, and R¹⁵ in formula (2) preferably each independently represent a hydrogen atom, a phenyl, a biphenylyl, a naphthyl, an anthracenyl, a phenanthrenyl, a monovalent group having a structure represented by the above formula (2-Ar1), (2-Ar2), (2-Ar3), (2-Ar4), or (2-Ar5) (the monovalent group having the structure may be bonded to the dibenzochrysene skeleton in the above formula (2) via a phenylene, a biphenylene, a naphthylene, an anthracenylene, a methylene, an ethylene, —OCH₂CH₂—, —CH₂CH₂O—, or —OCH₂CH₂O—), a methyl, an ethyl, a propyl, or a butyl.

In the compound represented by general formula (2), R¹, R², R⁴, R⁵, R⁷, R⁸, R⁹, R¹⁰, R¹², R¹³, R¹⁵, and R¹⁶ more preferably each represent a hydrogen atom. In this case, at least one (preferably one or two, more preferably one) of R³, R⁶, R¹¹, and R¹⁴ in formula (2) represents a monovalent group having a structure of the above formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—, and

a group other than the at least one (that is, a group located at a position other than the substitution position of the monovalent group having the above structure) represents a hydrogen atom, a phenyl, a biphenylyl, a naphthyl, an anthracenyl, a methyl, an ethyl, a propyl, or a butyl, while at least one hydrogen atom in these may be substituted by a phenyl, a biphenylyl, a naphthyl, an anthracenyl, a methyl, an ethyl, a propyl, or a butyl.

When a monovalent group having a structure represented by any one of the formulas (2-Ar1) to (2-Ar5) is selected as R², R³, R⁶, R⁷, R¹⁰, R¹¹, R¹⁴, or R¹⁵ in formula (2), at least one hydrogen atom in the structure may be bonded to any of R¹ to R¹⁶ in formula (2) to form a single bond.

All or some of hydrogen atoms in the compound represented by formula (2) may be substituted by a halogen atom, a cyano, or a deuterium atom. For example, in formula (2), a hydrogen atom in an aryl, a heteroaryl, a diarylamino, a diheteroarylamino, an arylheteroarylamino, an alkyl, an alkenyl, an alkoxy, or an aryloxy in R¹ to R¹⁶, and a hydrogen atom in substituents for these can be substituted by a hydrogen atom, a cyano, or a deuterium atom. However, among these forms, a form in which all or some of hydrogen atoms in an aryl or a heteroaryl are substituted by a halogen atom, a cyano, or a deuterium atom can be mentioned. The halogen is fluorine, chlorine, bromine, or iodine, preferably fluorine, chlorine, or bromine, and more preferably chlorine.

More specific examples of the compound represented by formula (2) include compounds represented by the following structural formulas.

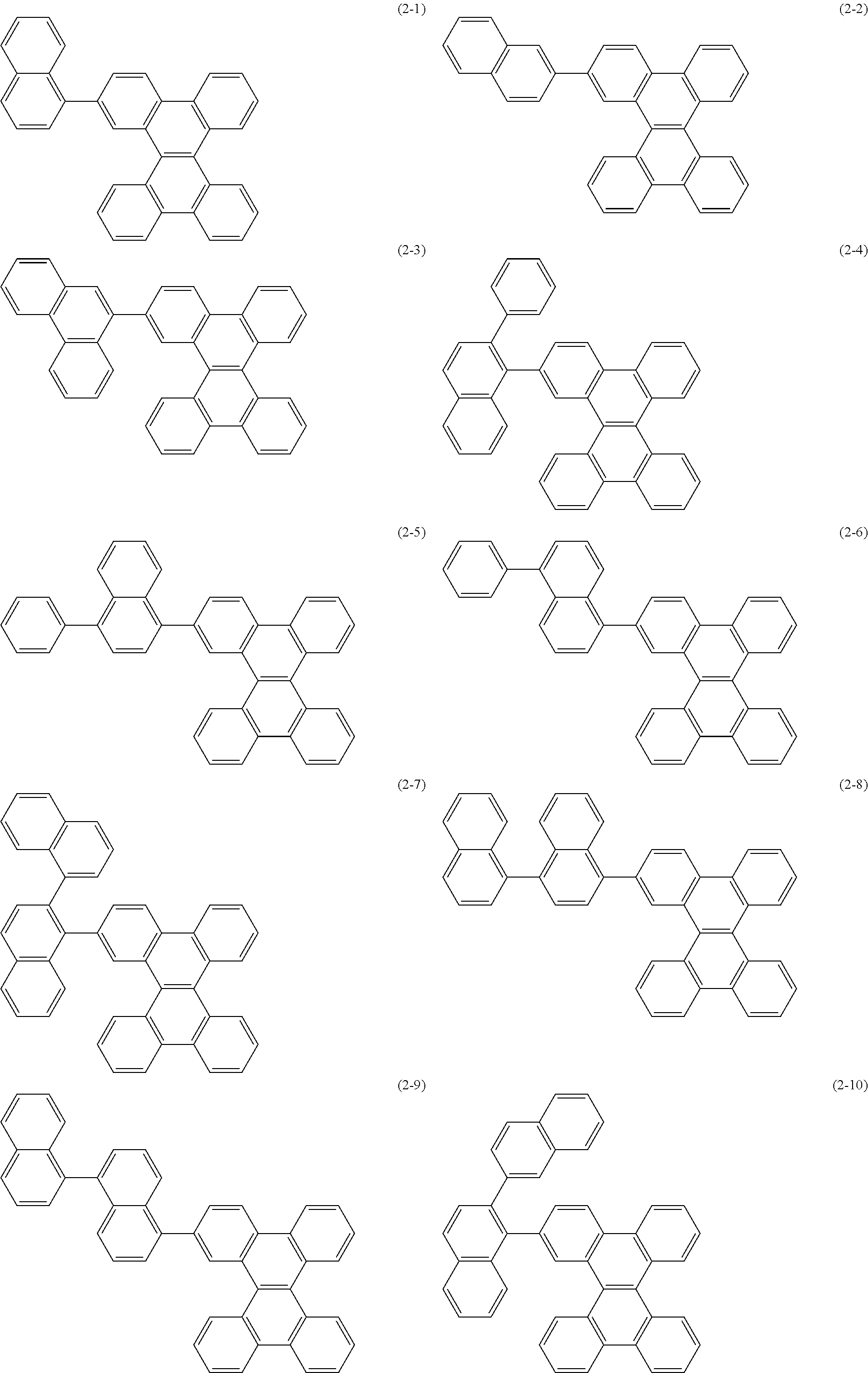

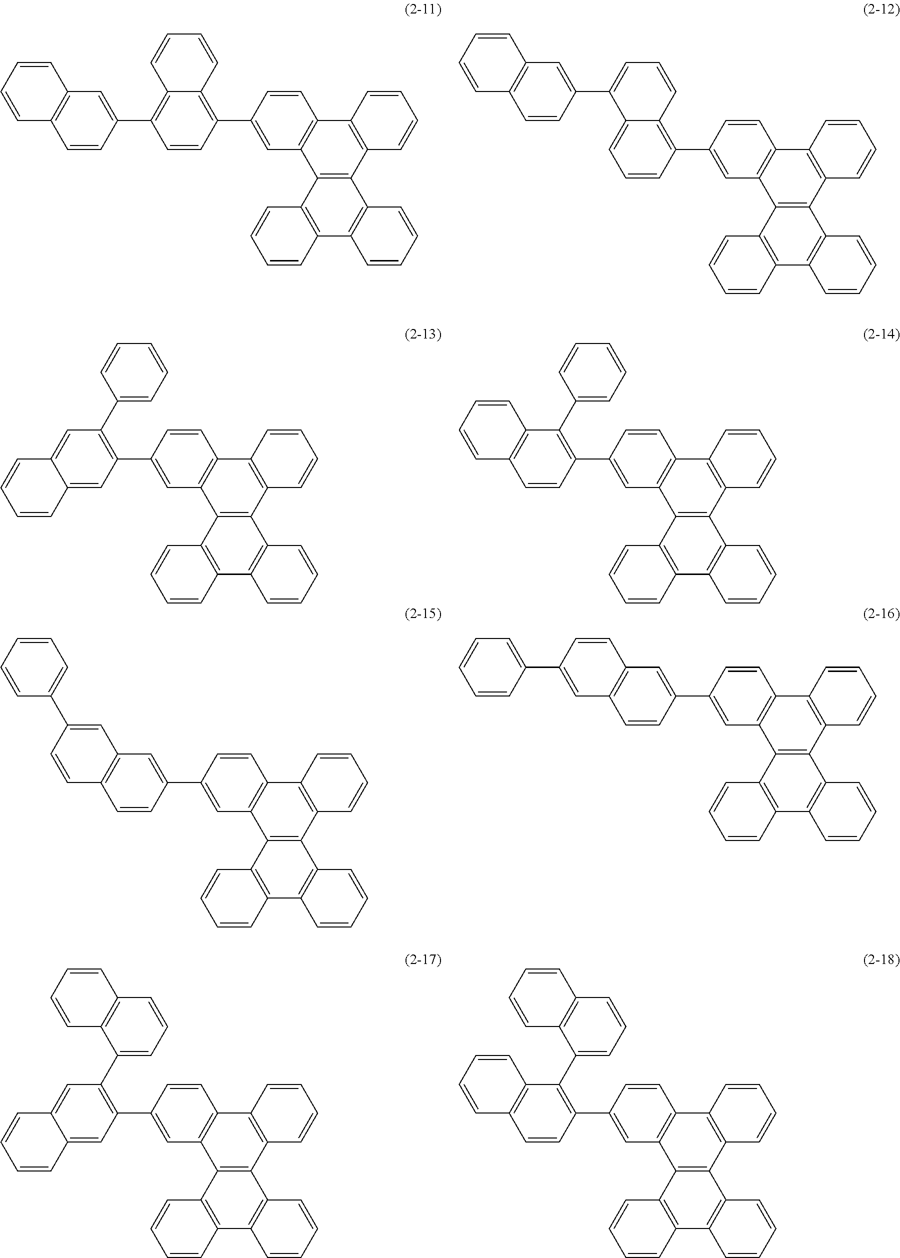

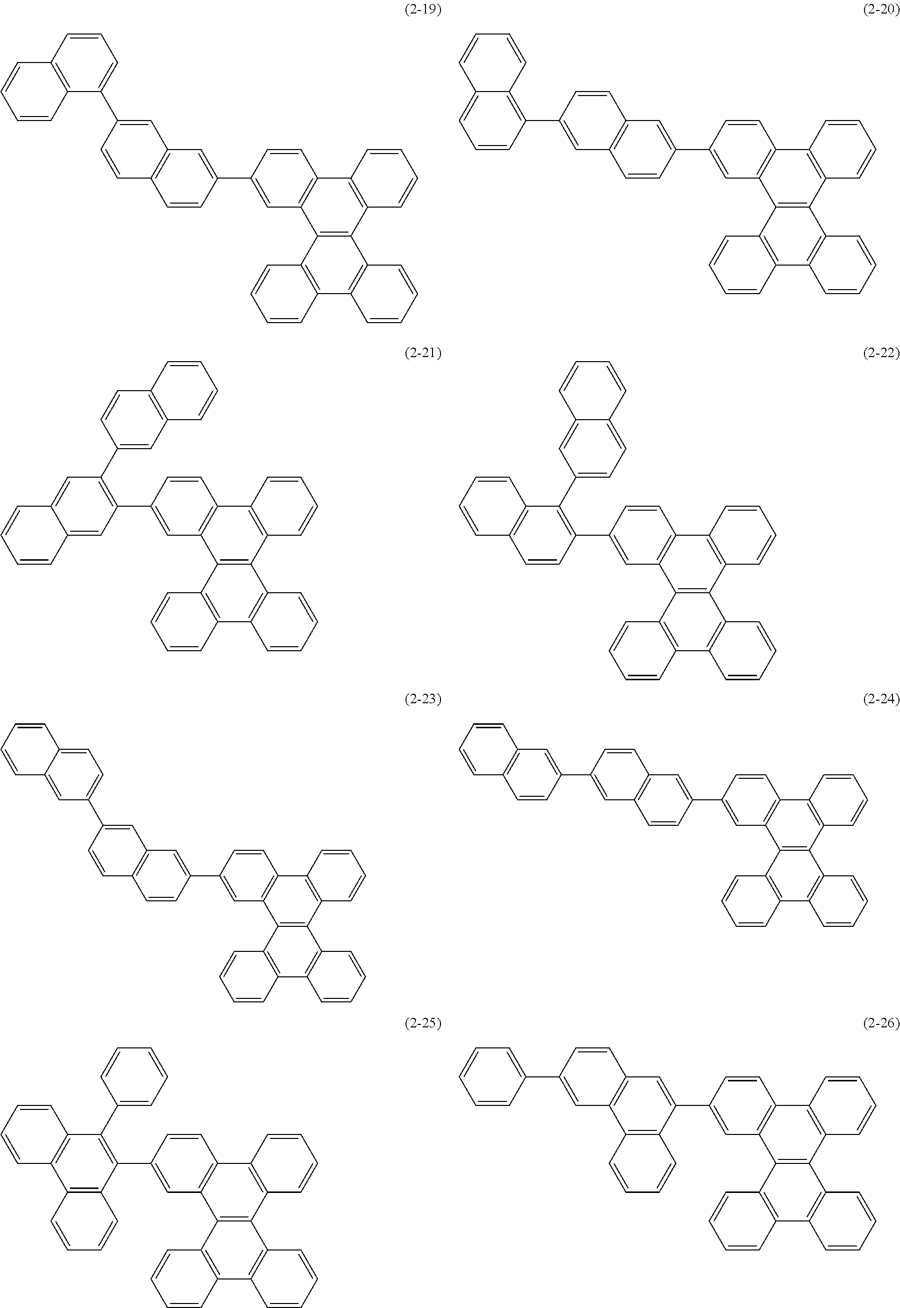

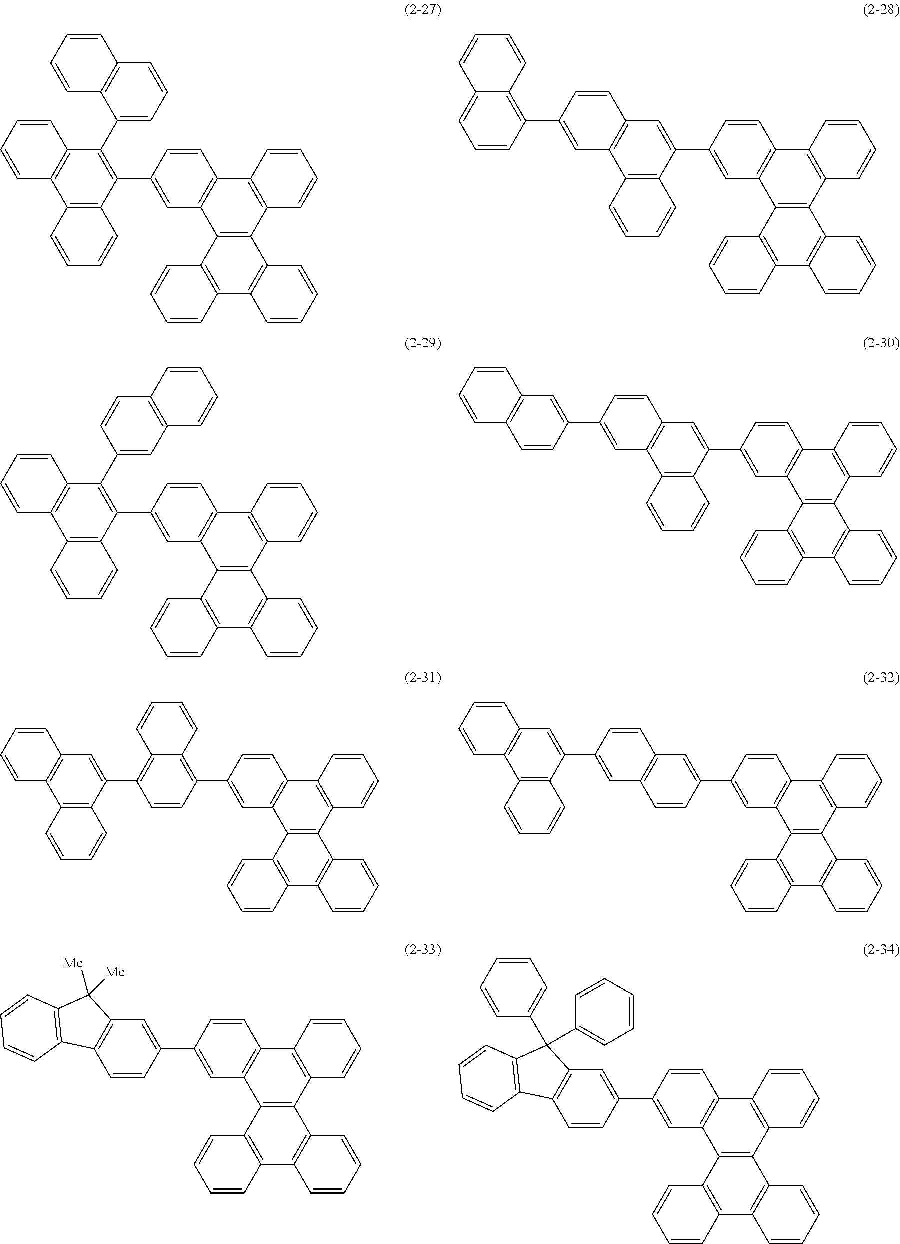

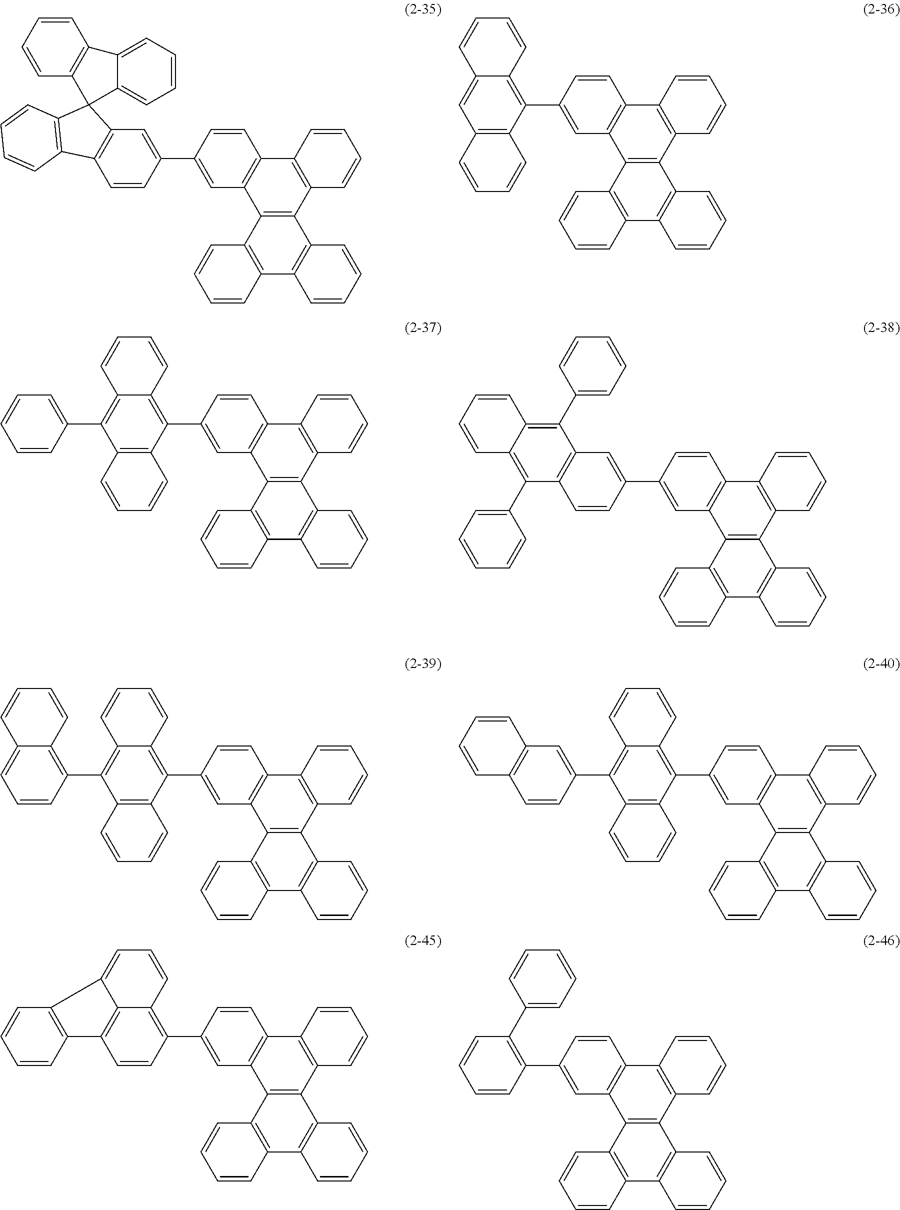

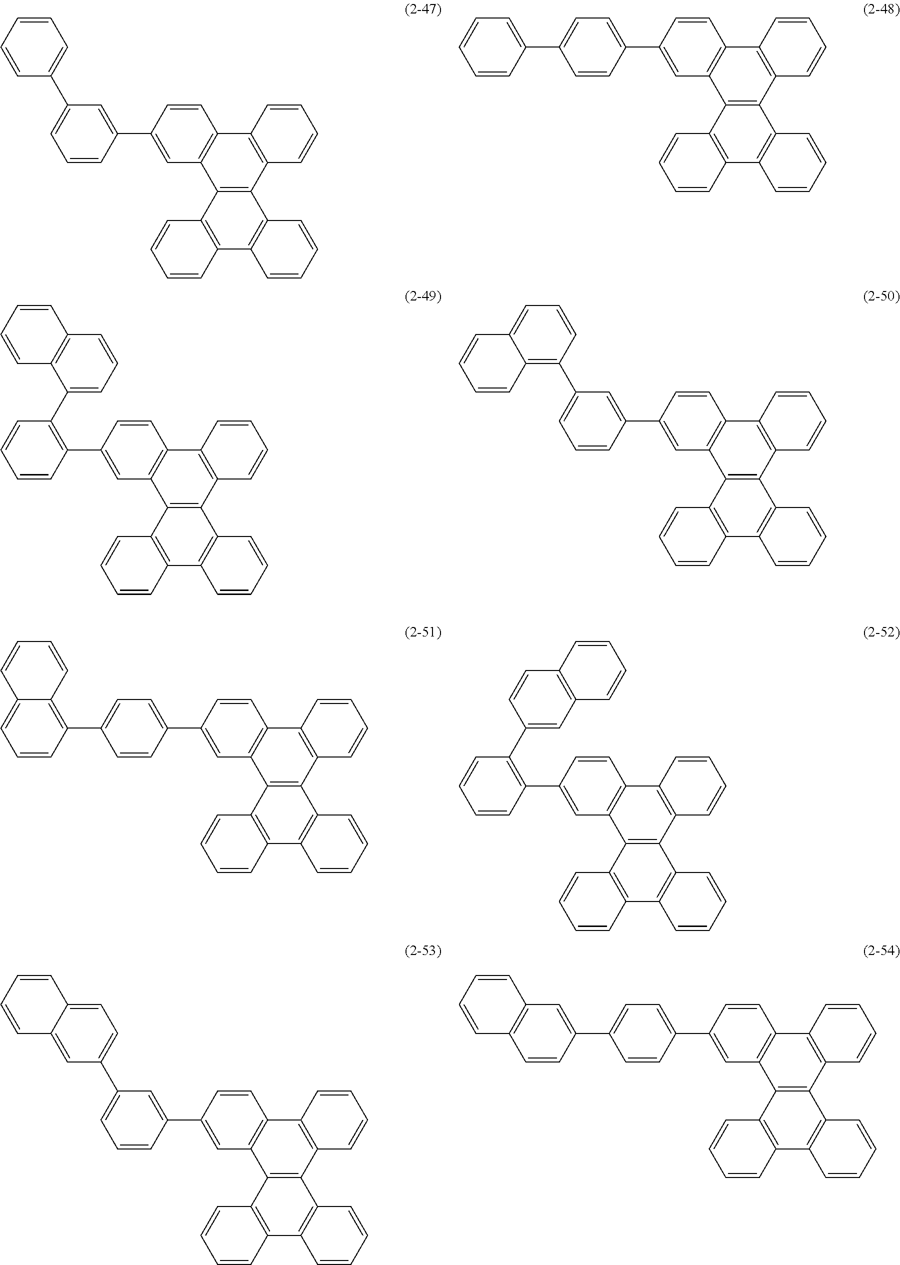

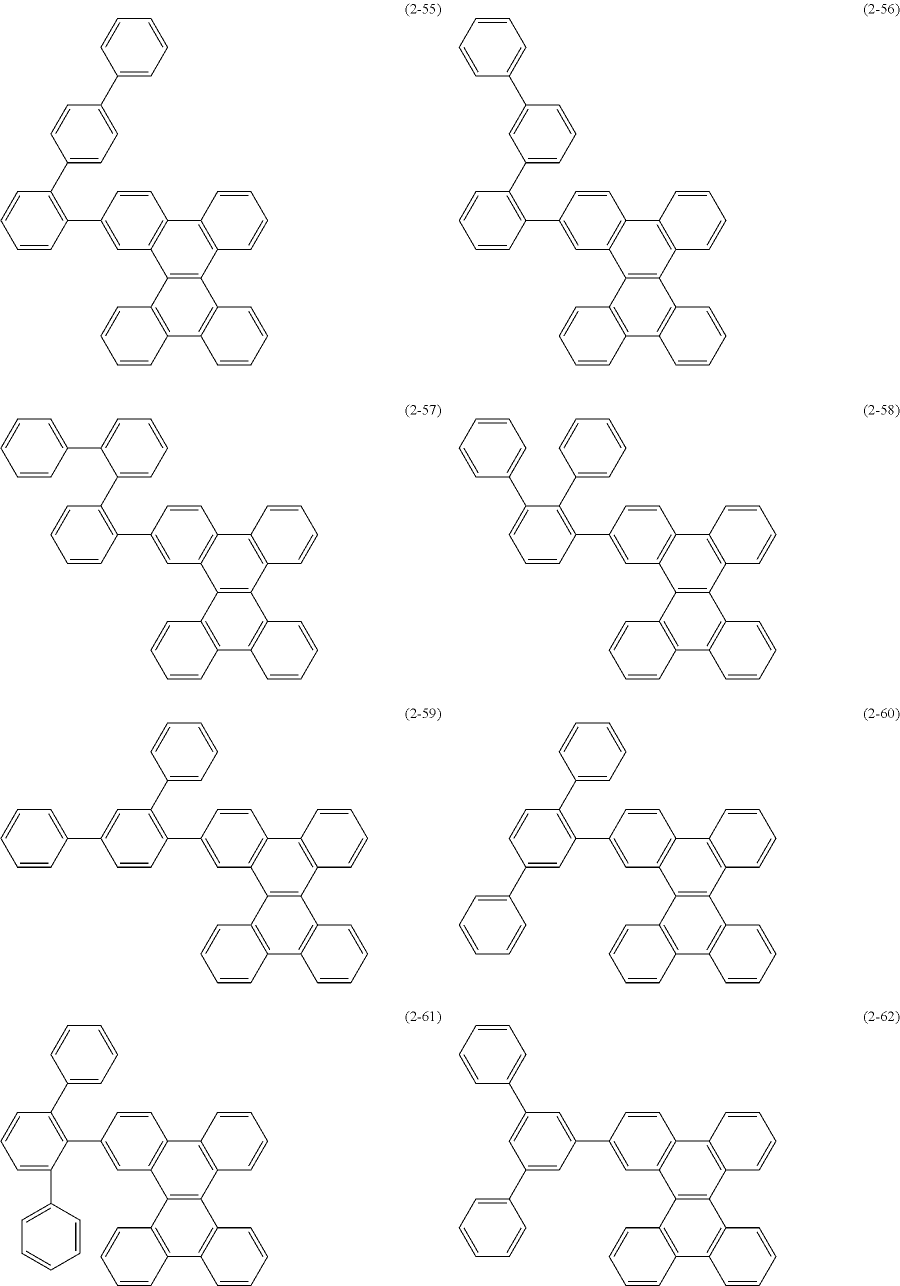

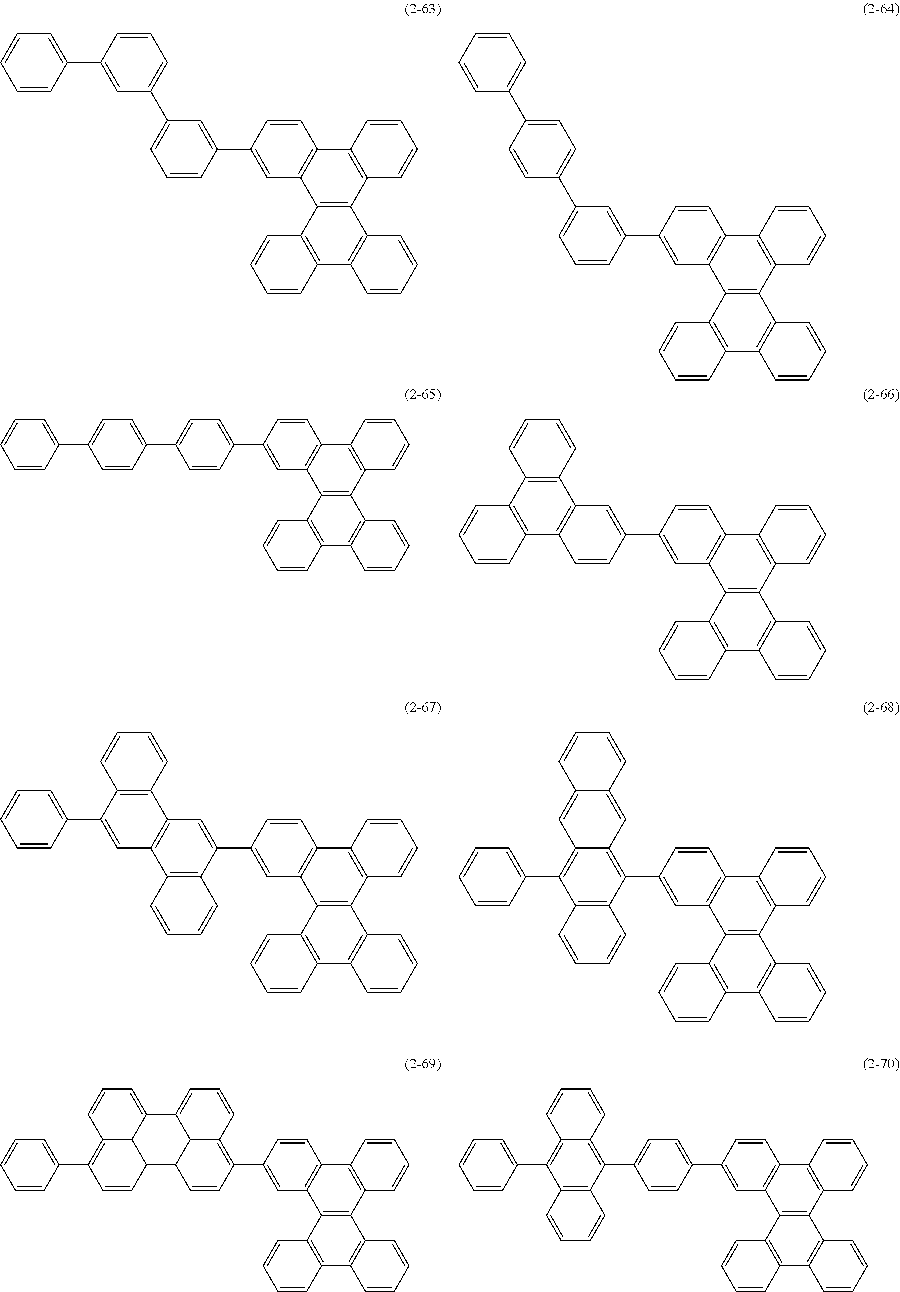

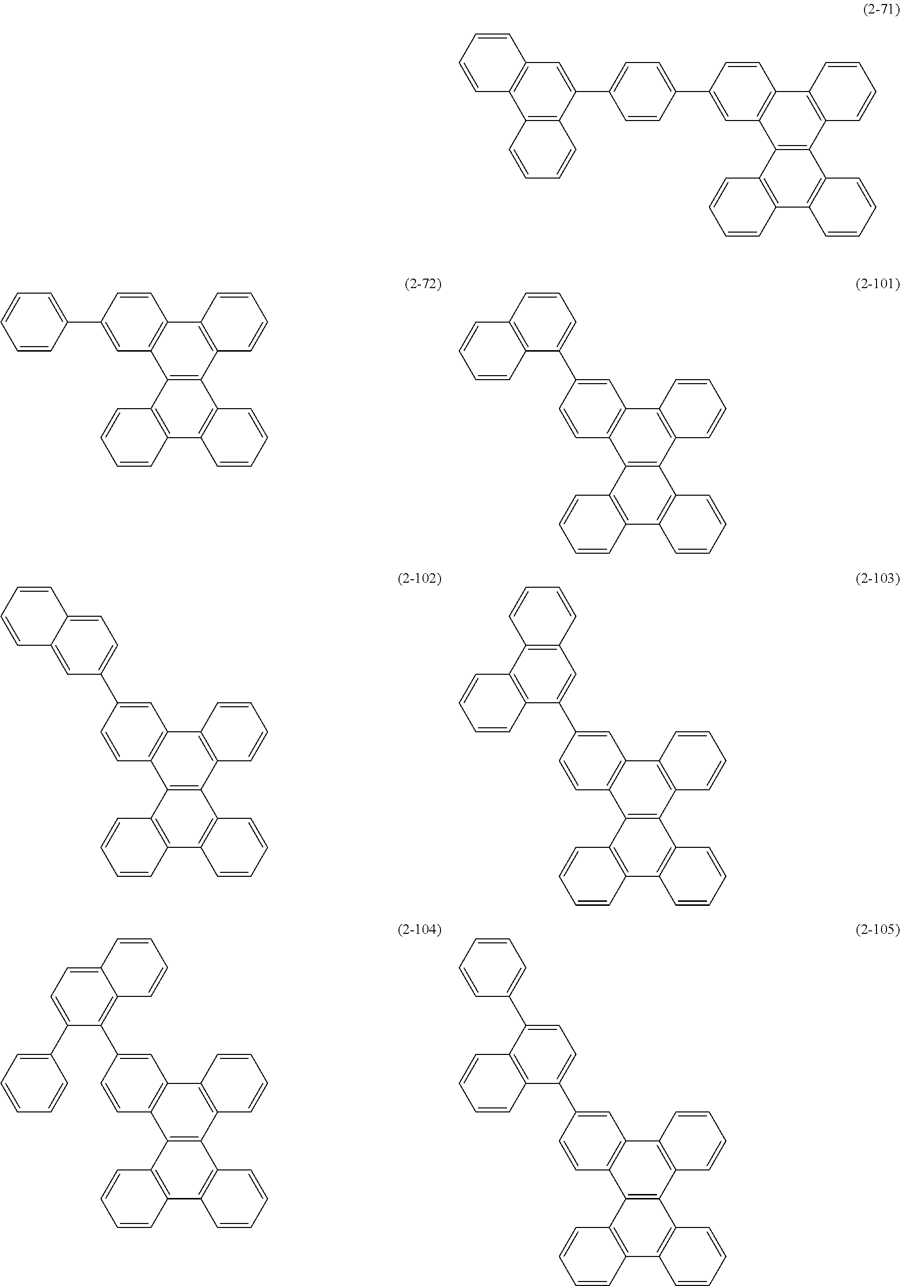

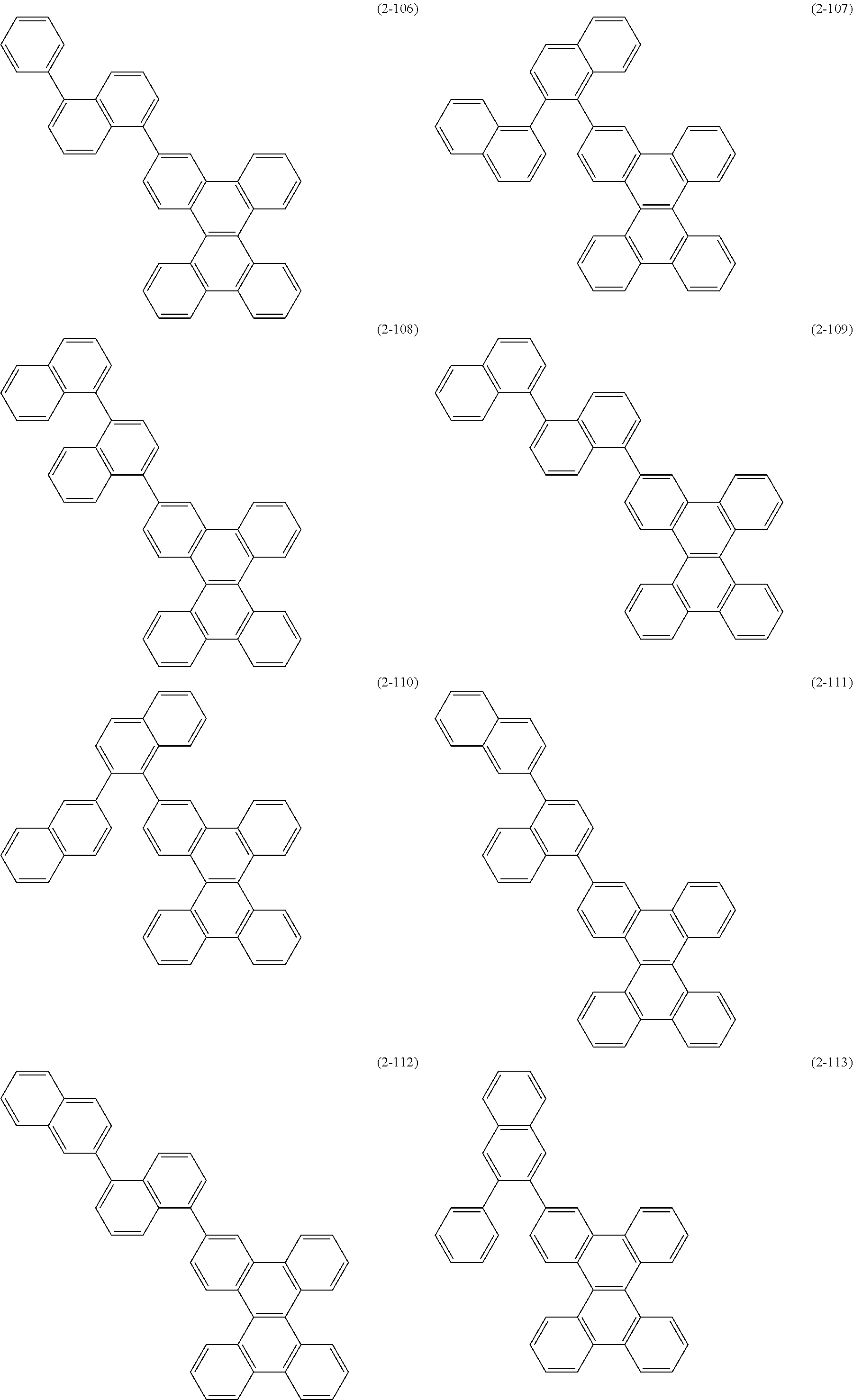

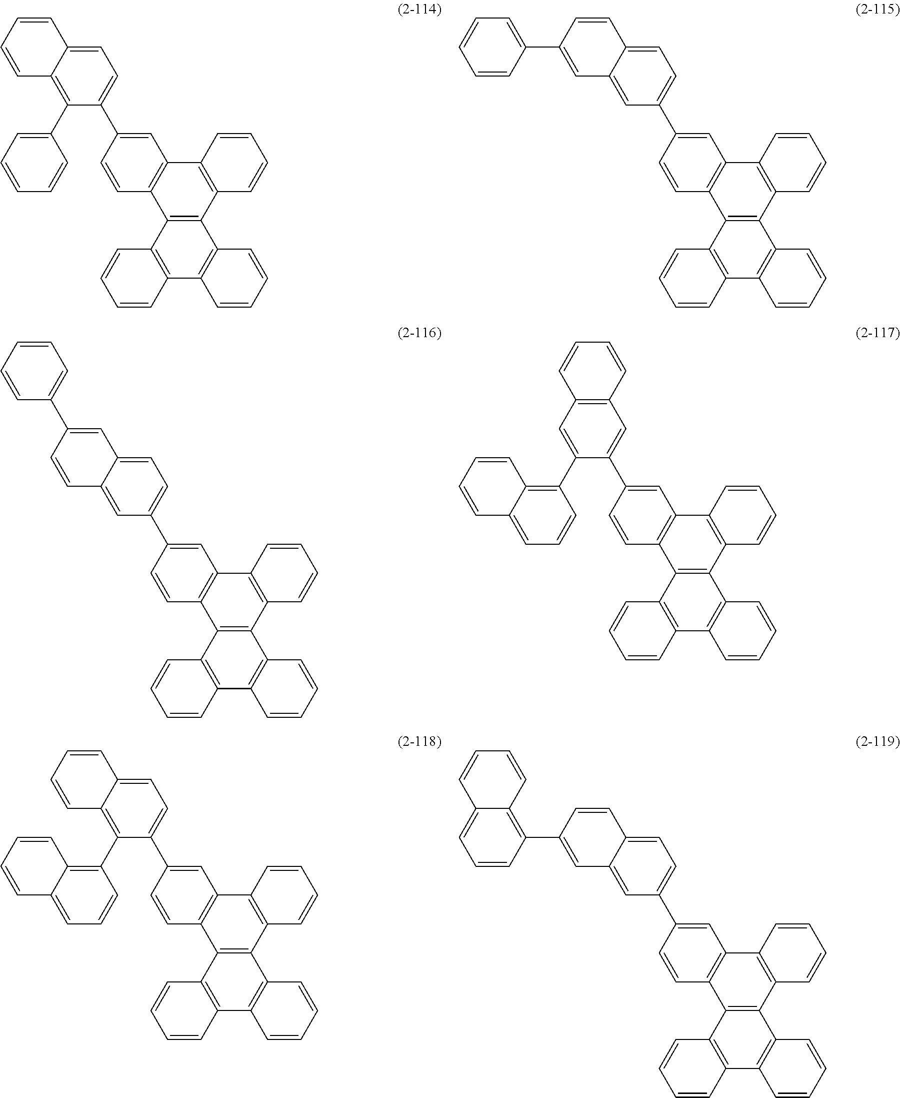

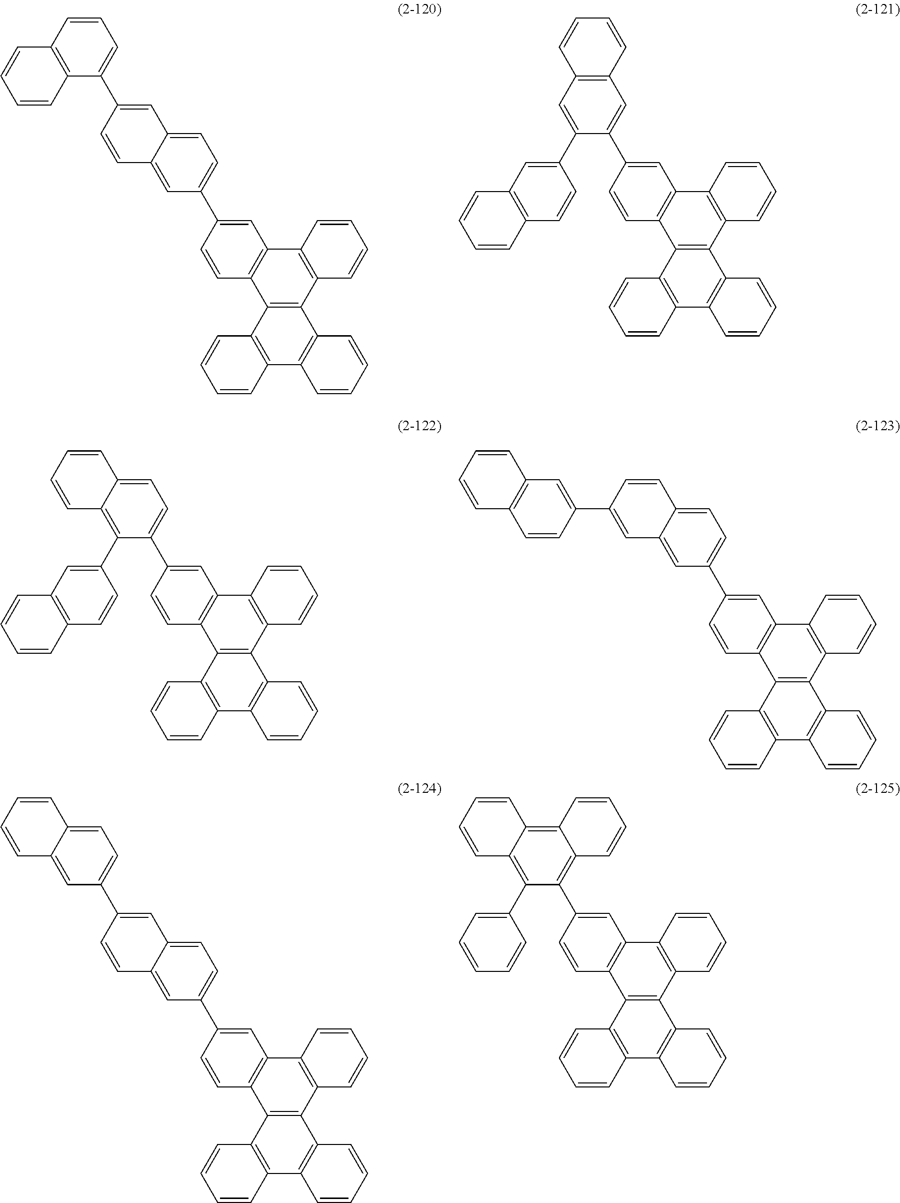

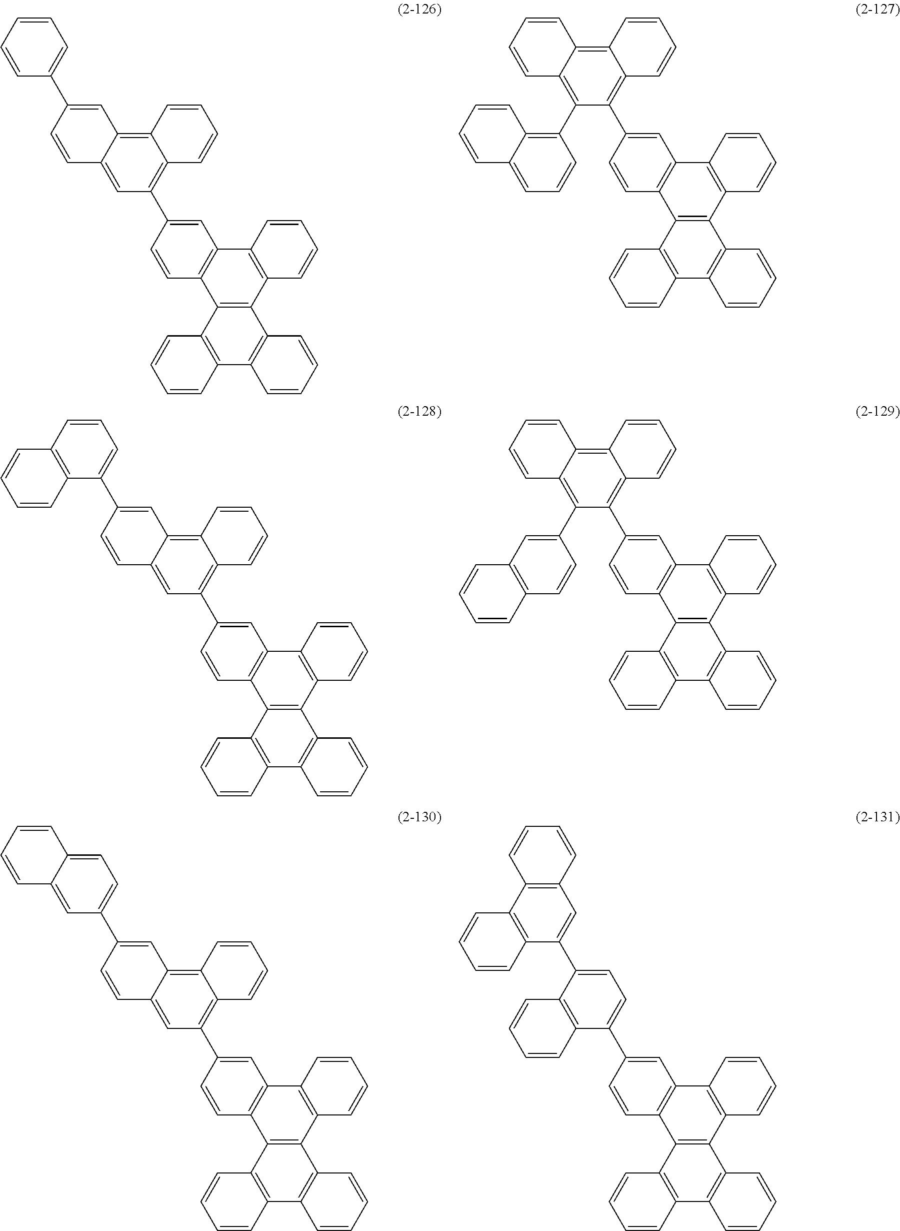

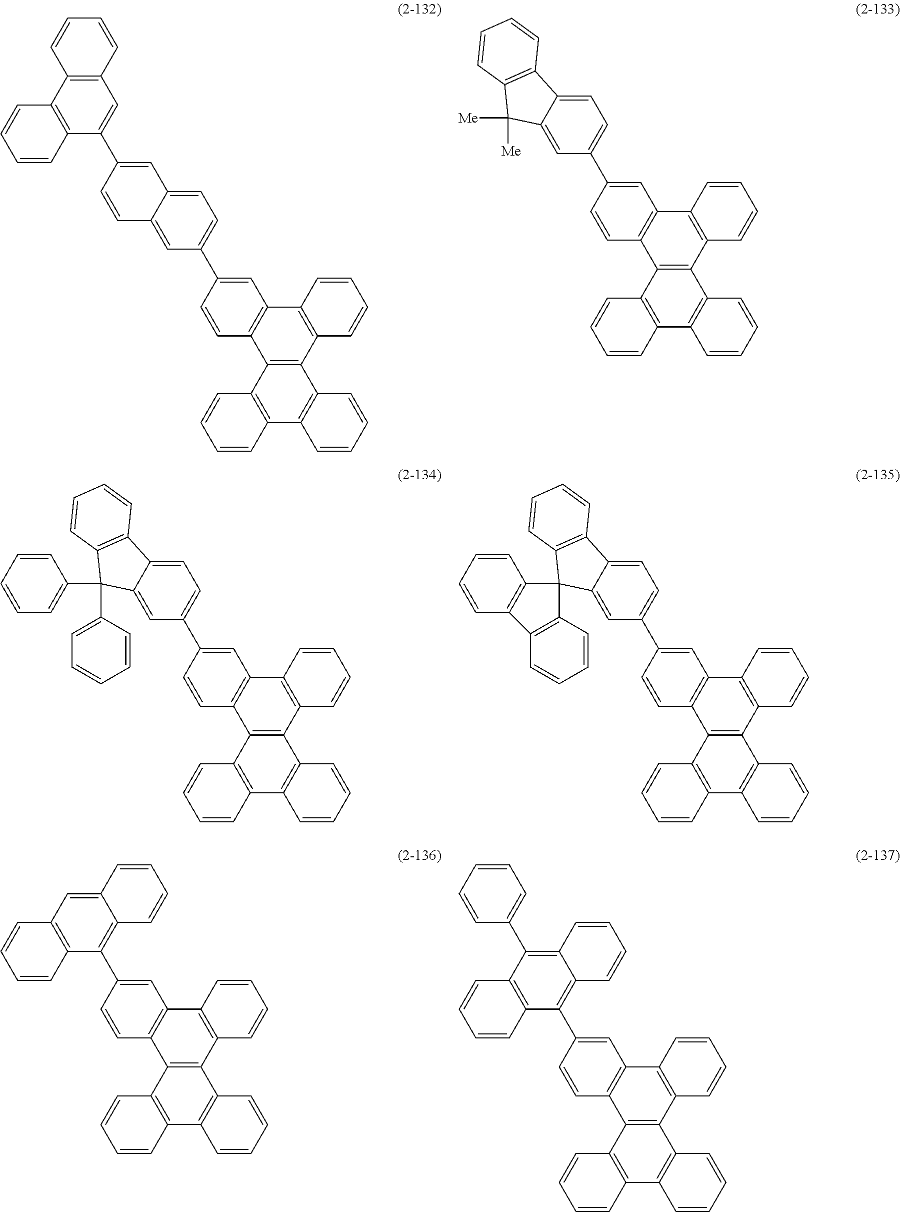

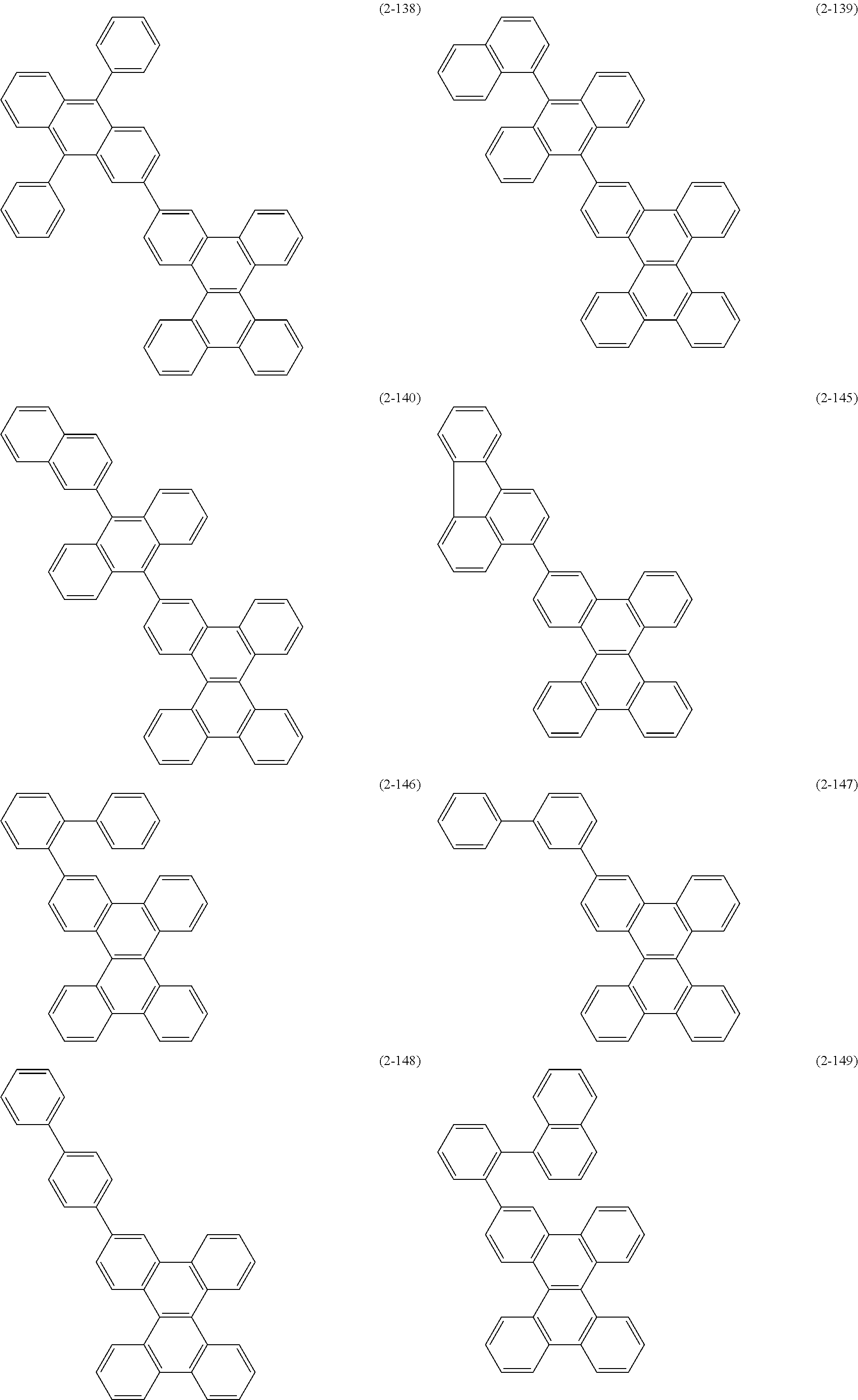

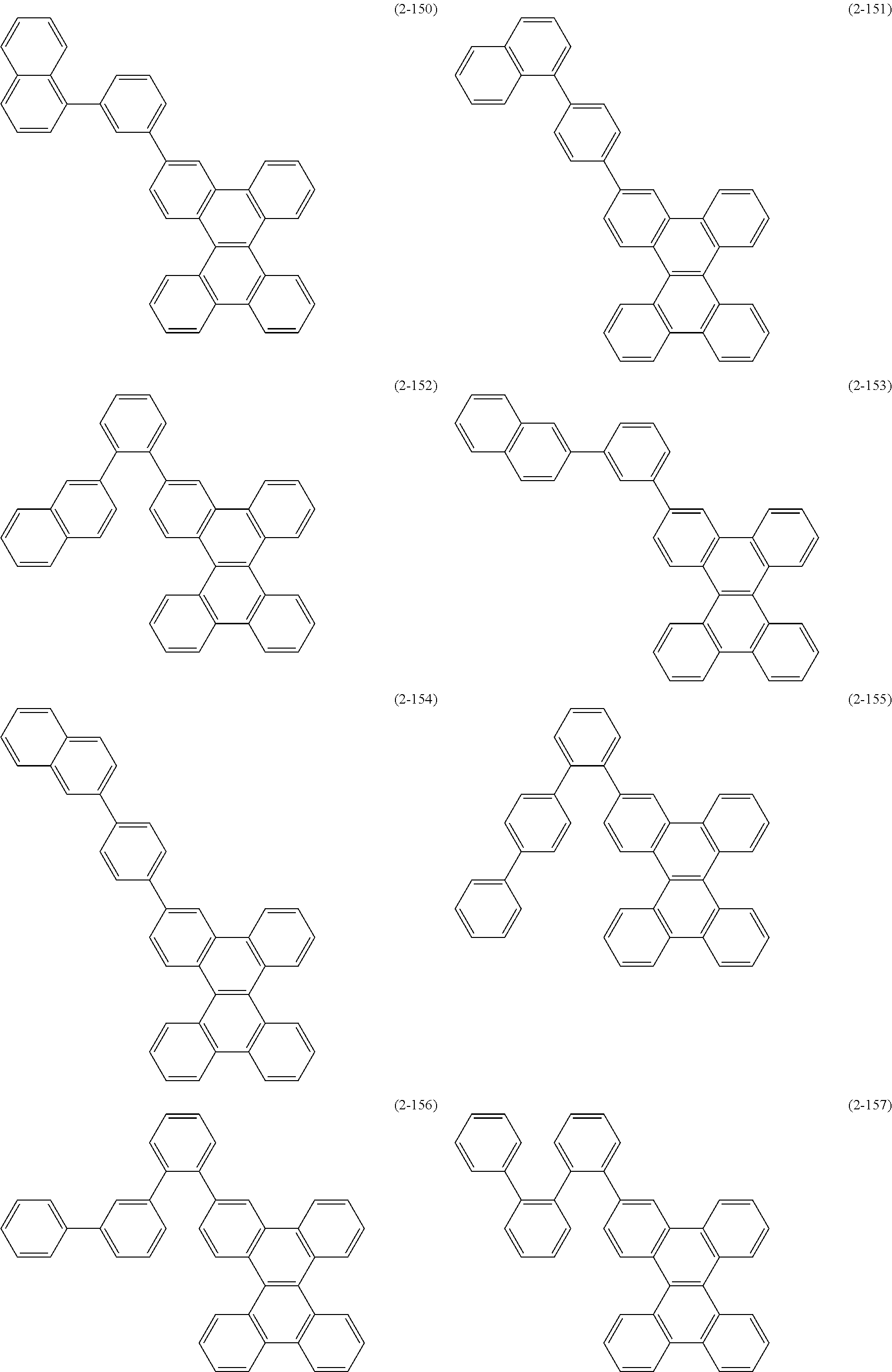

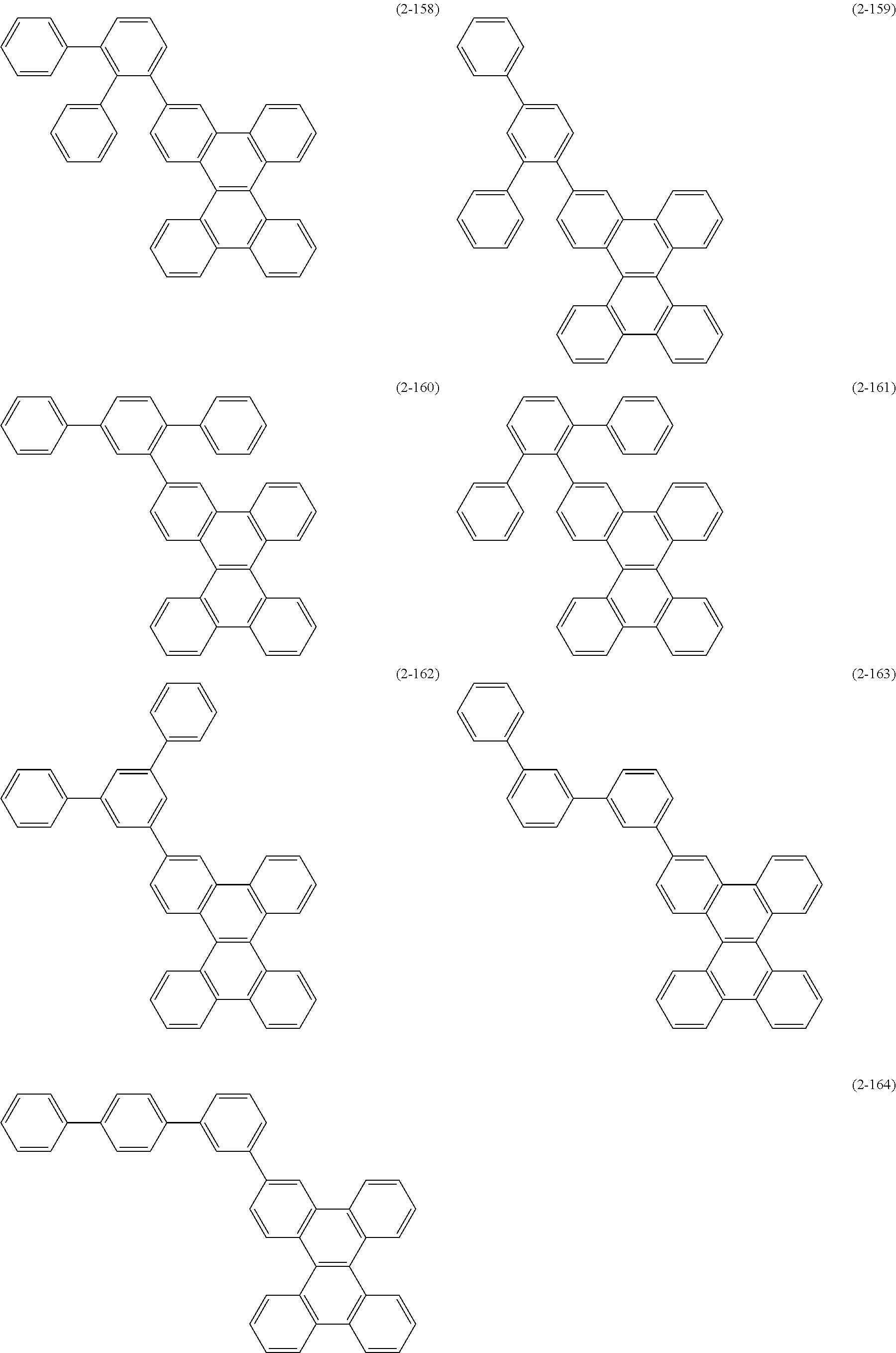

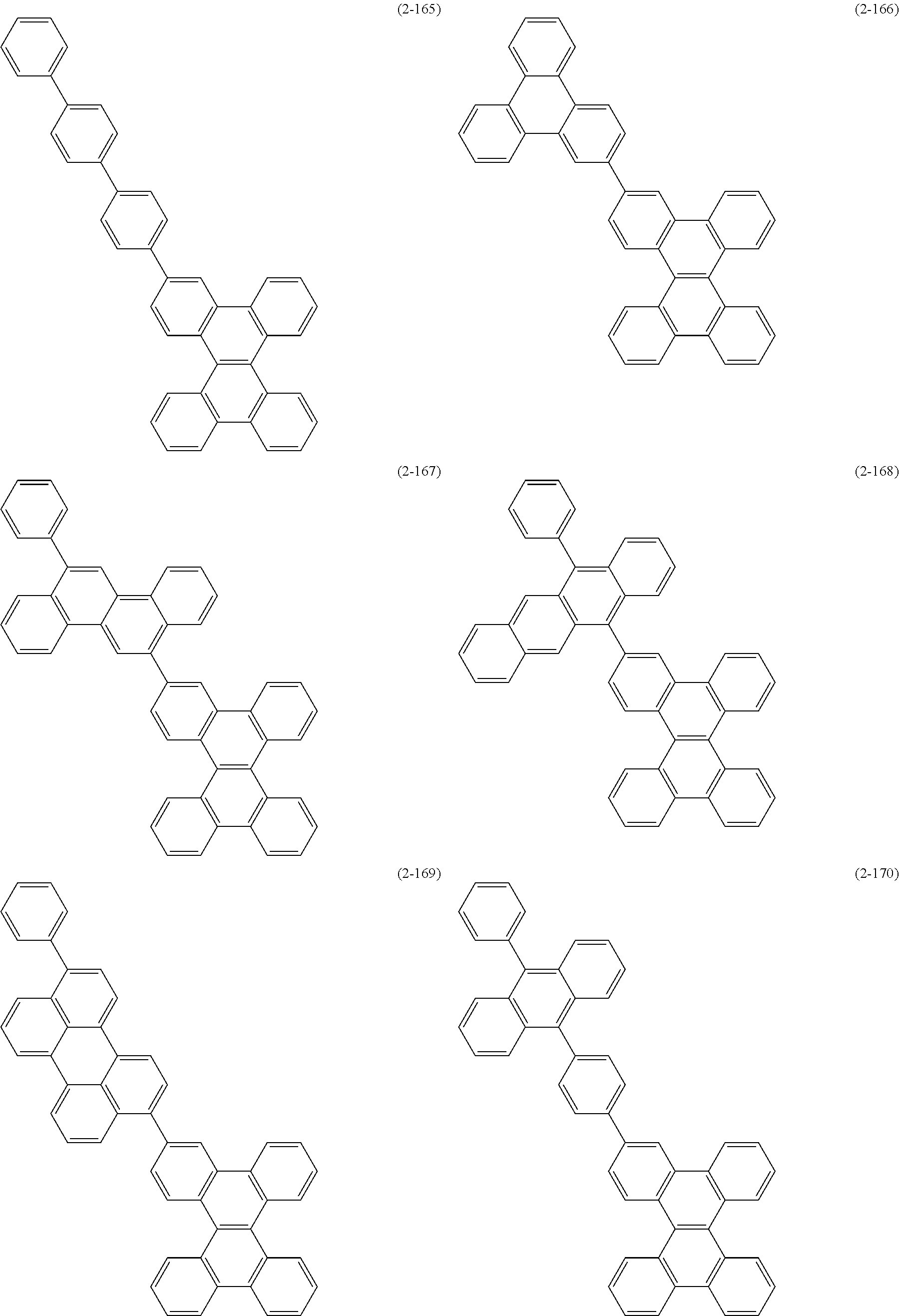

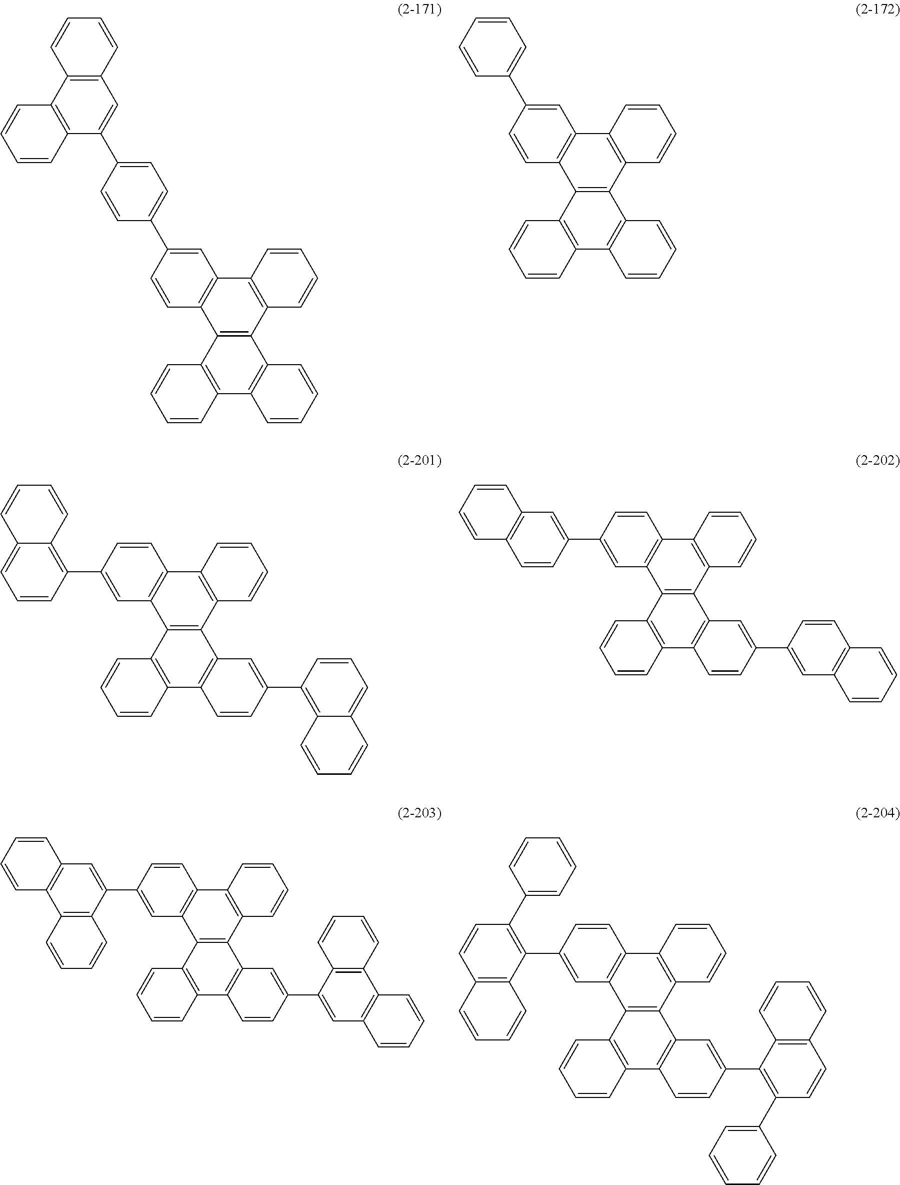

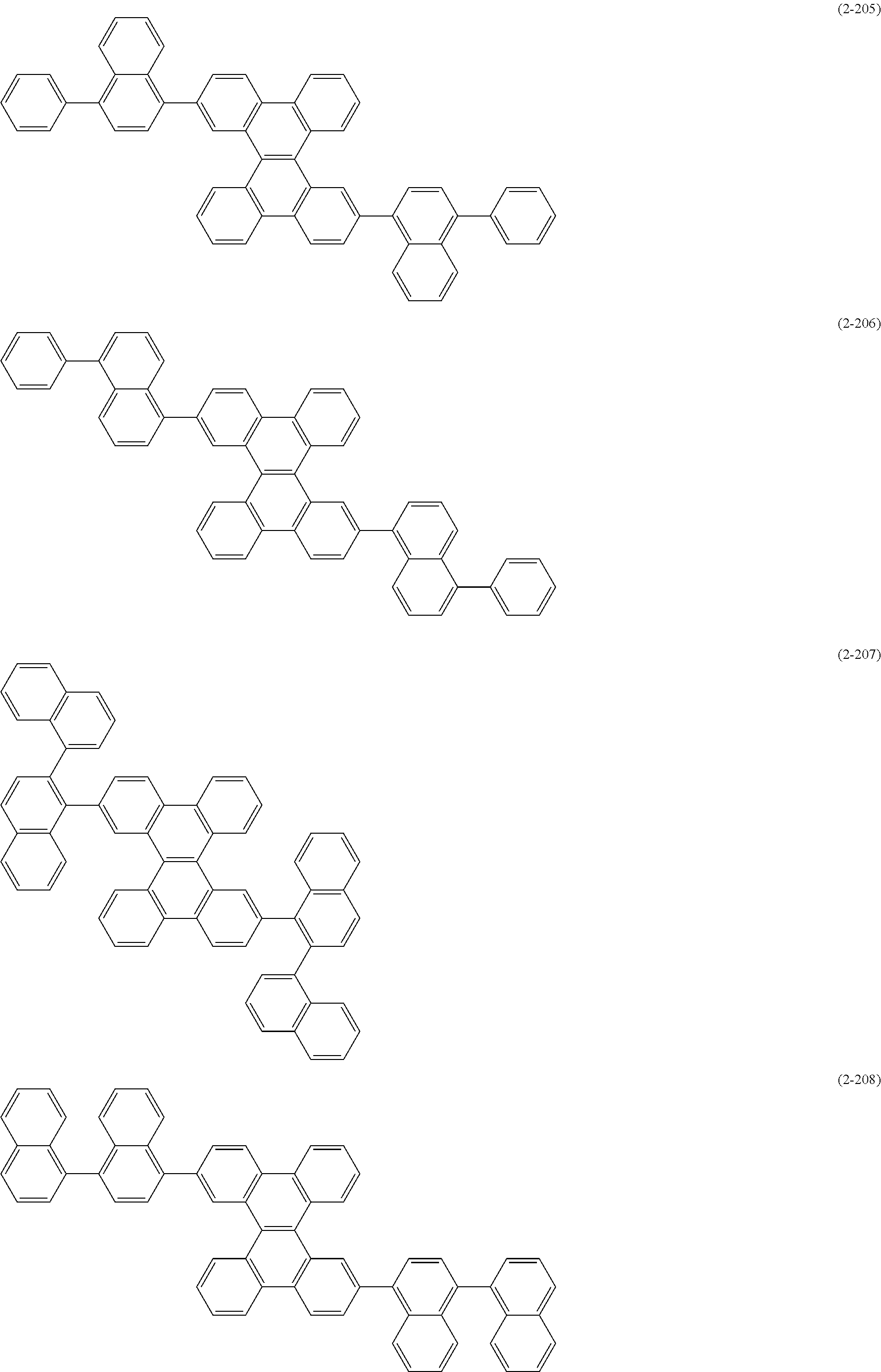

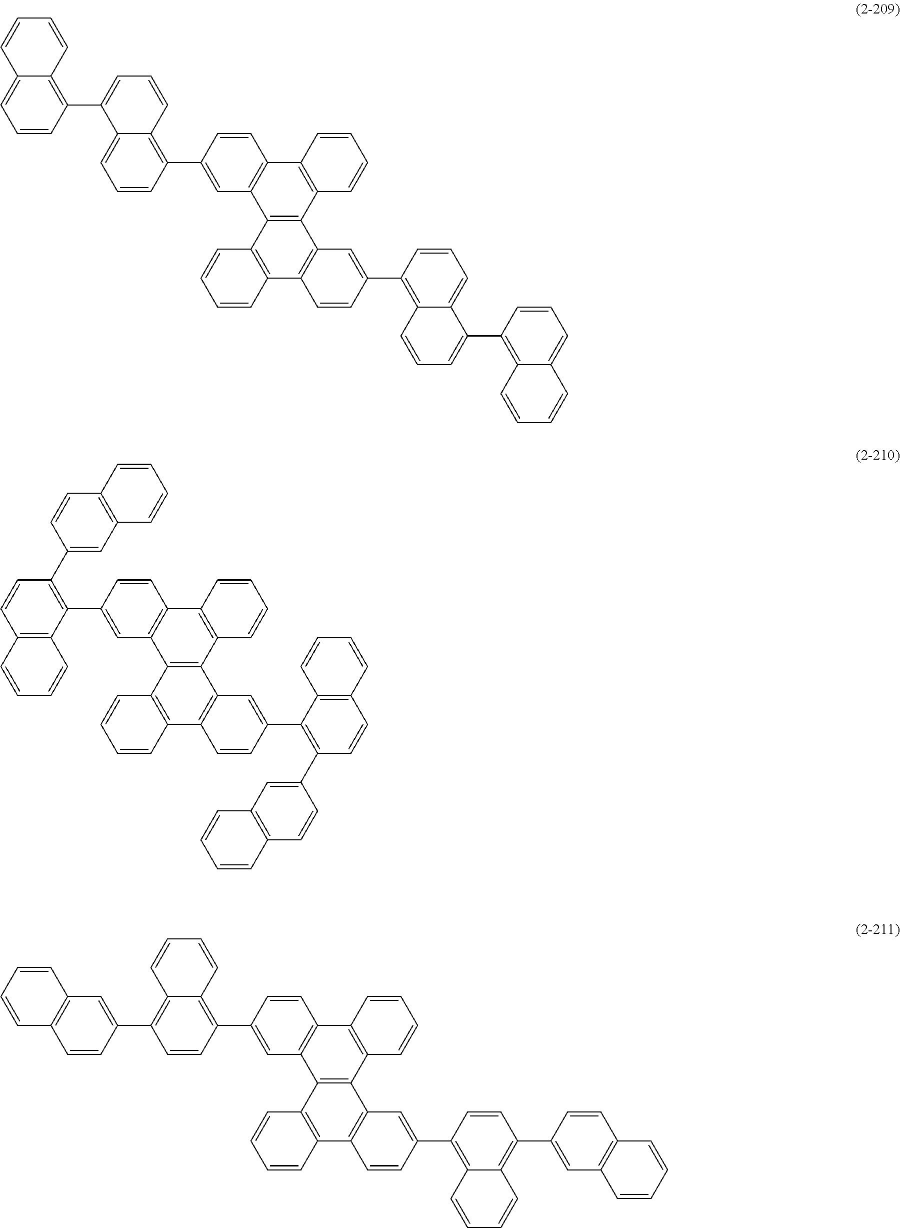

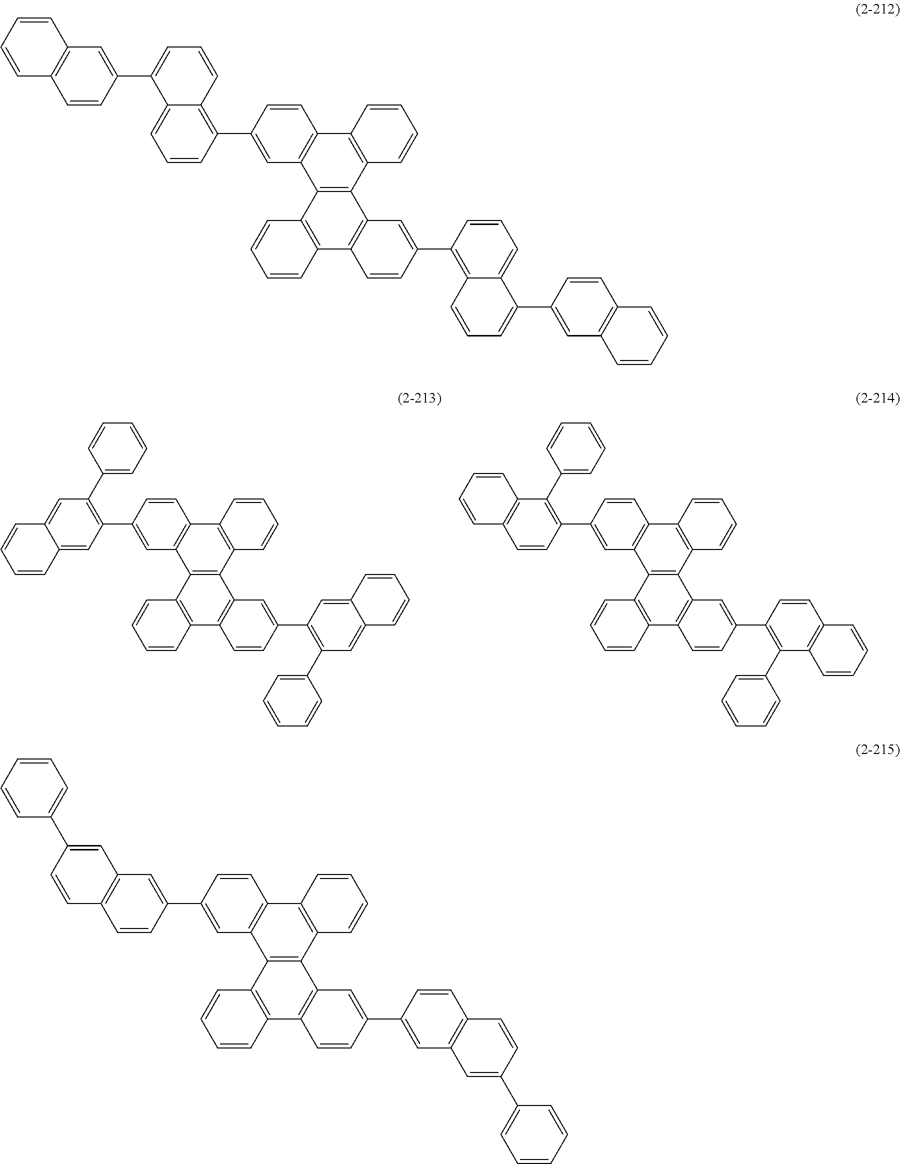

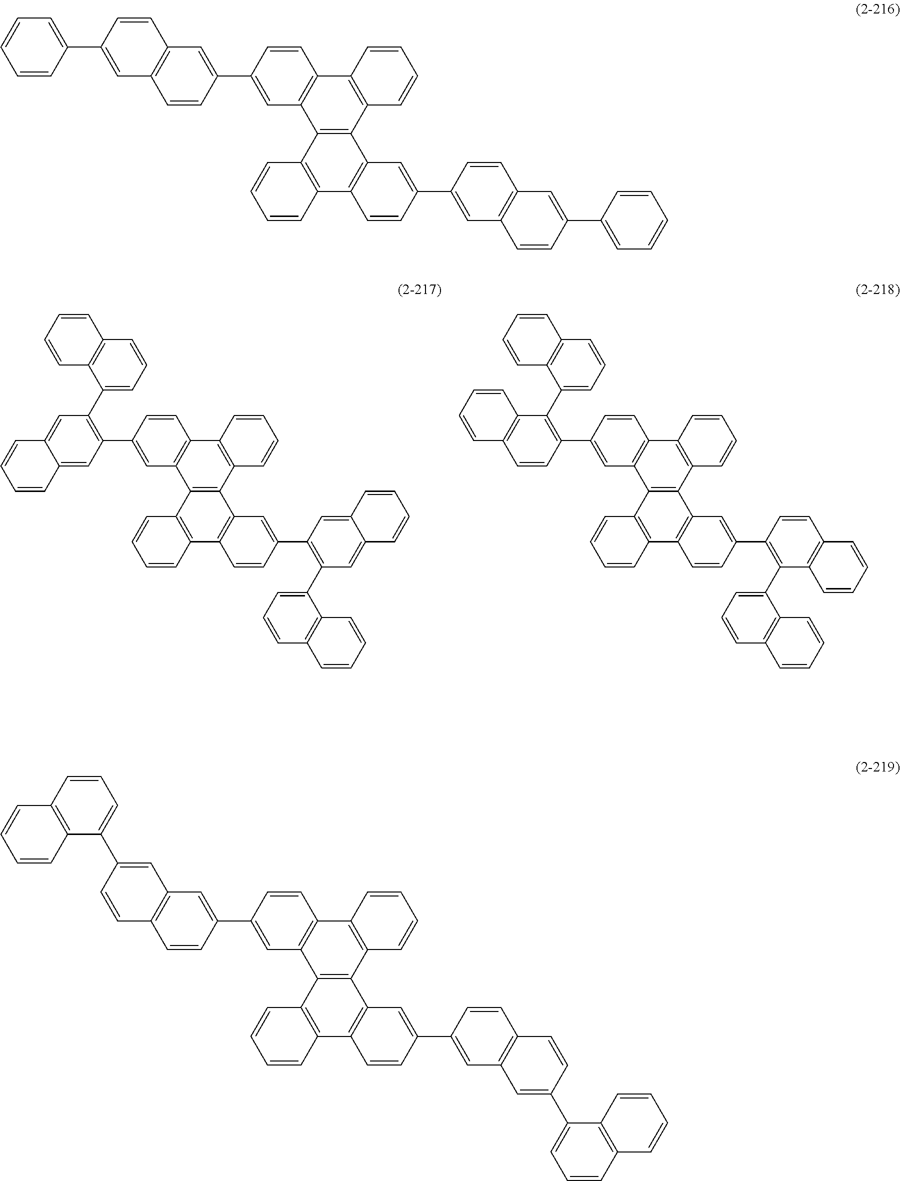

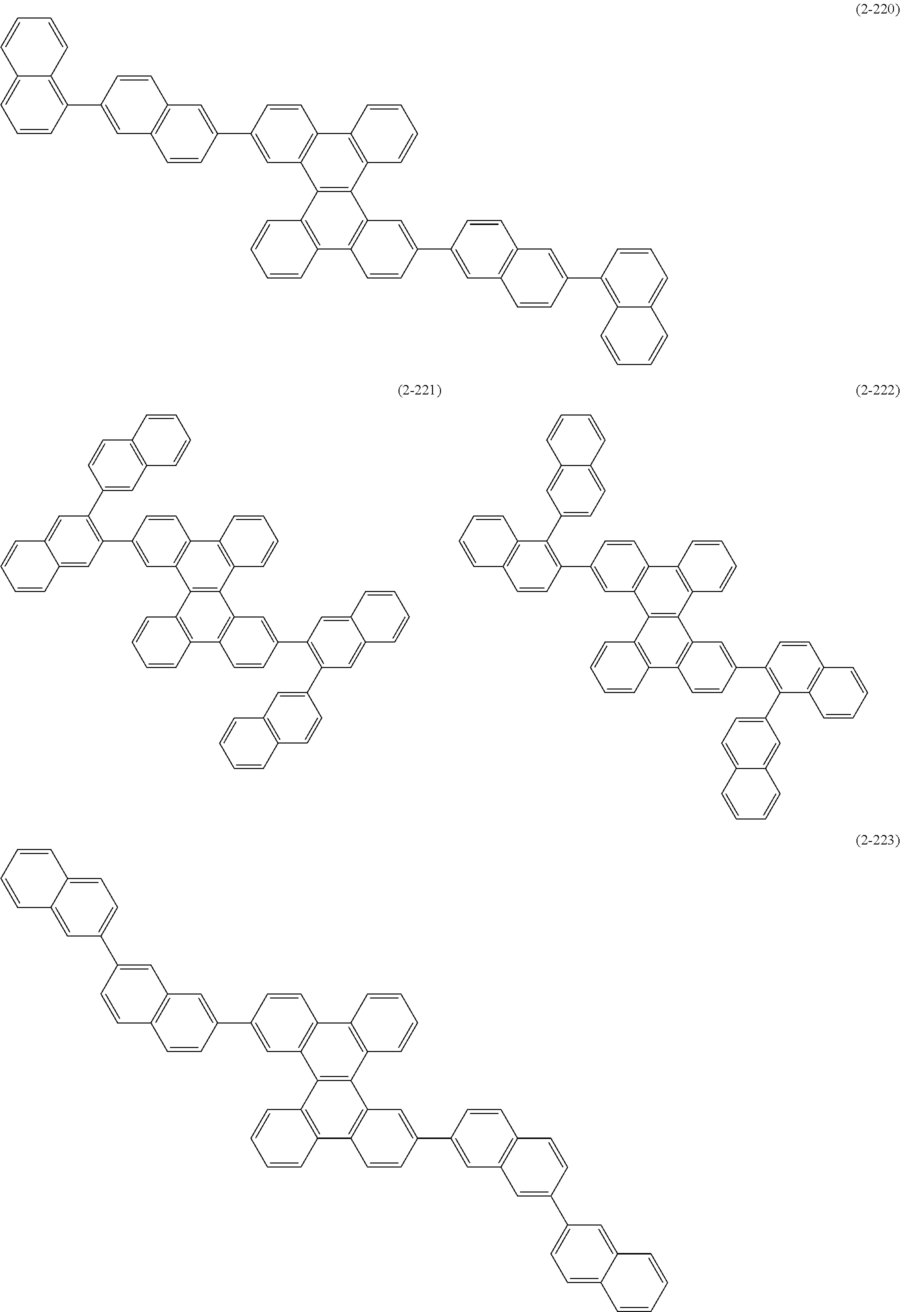

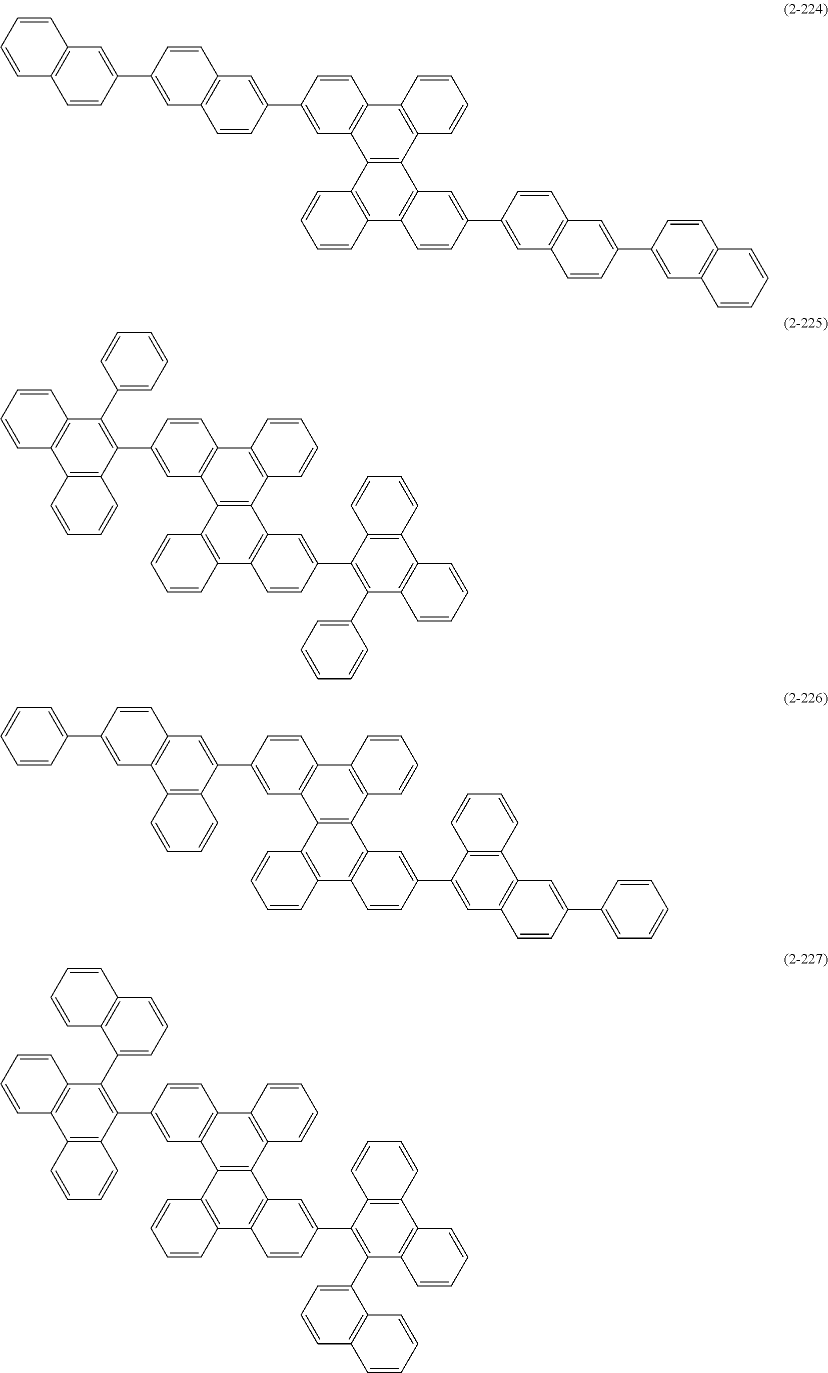

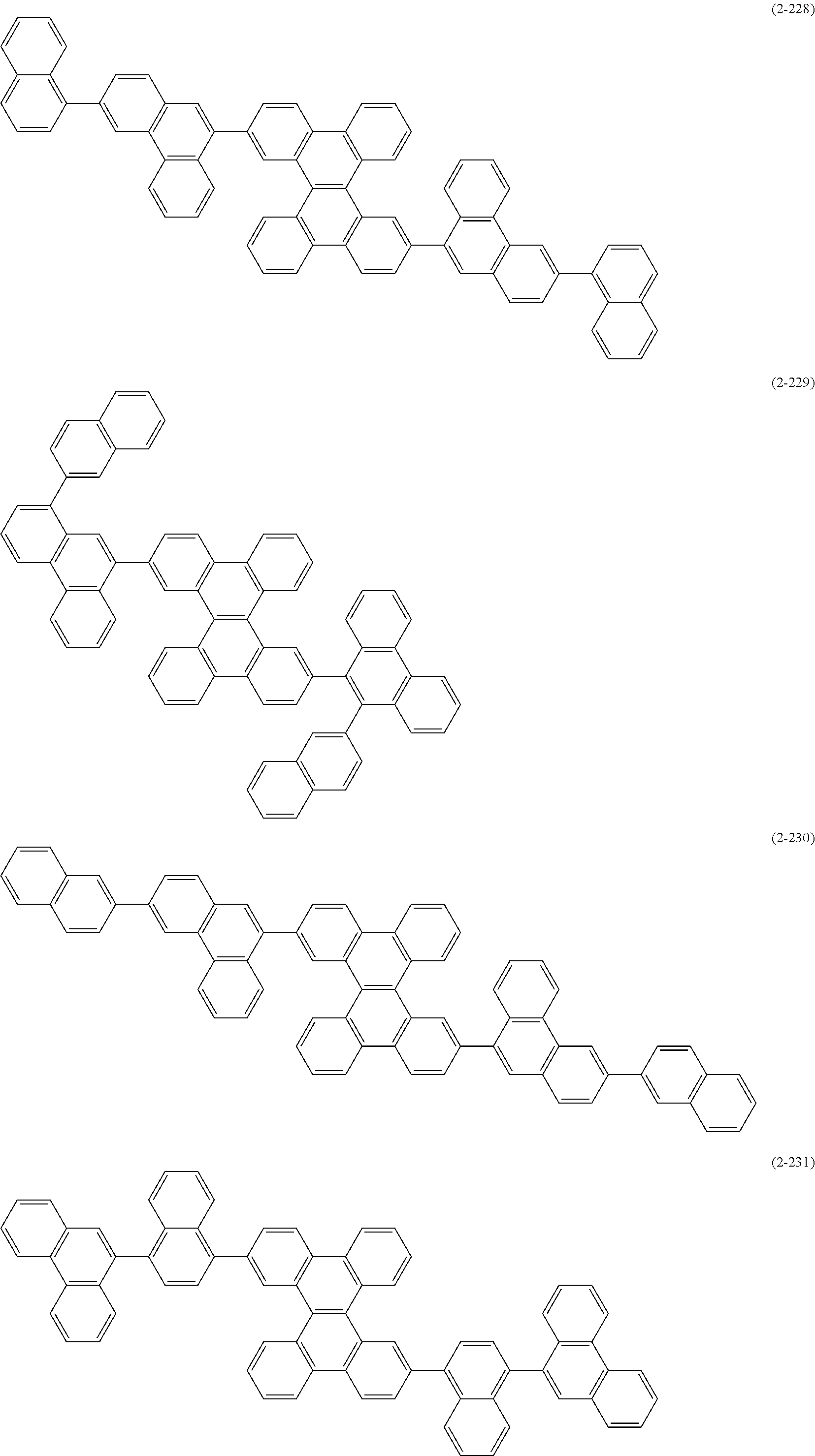

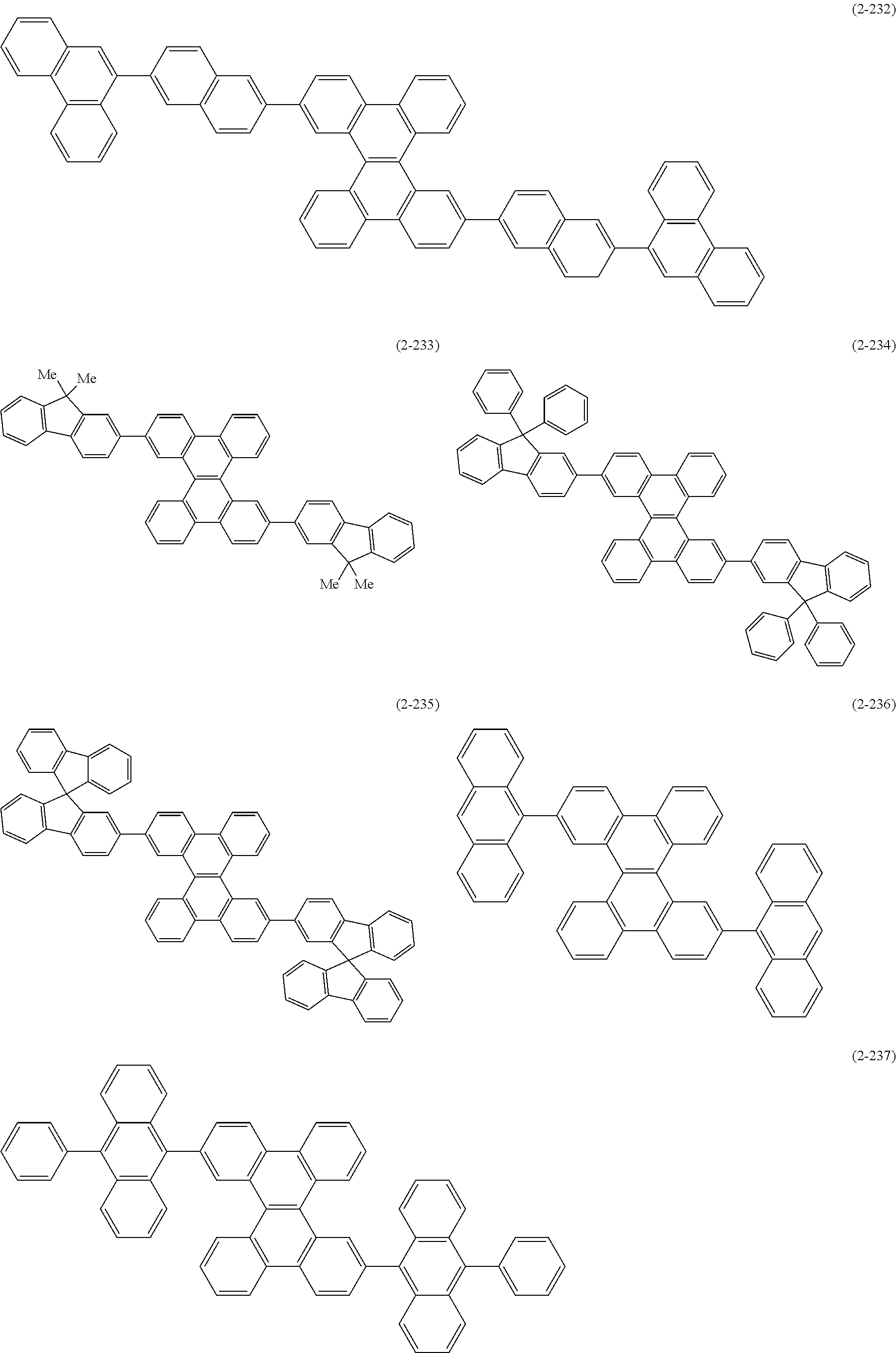

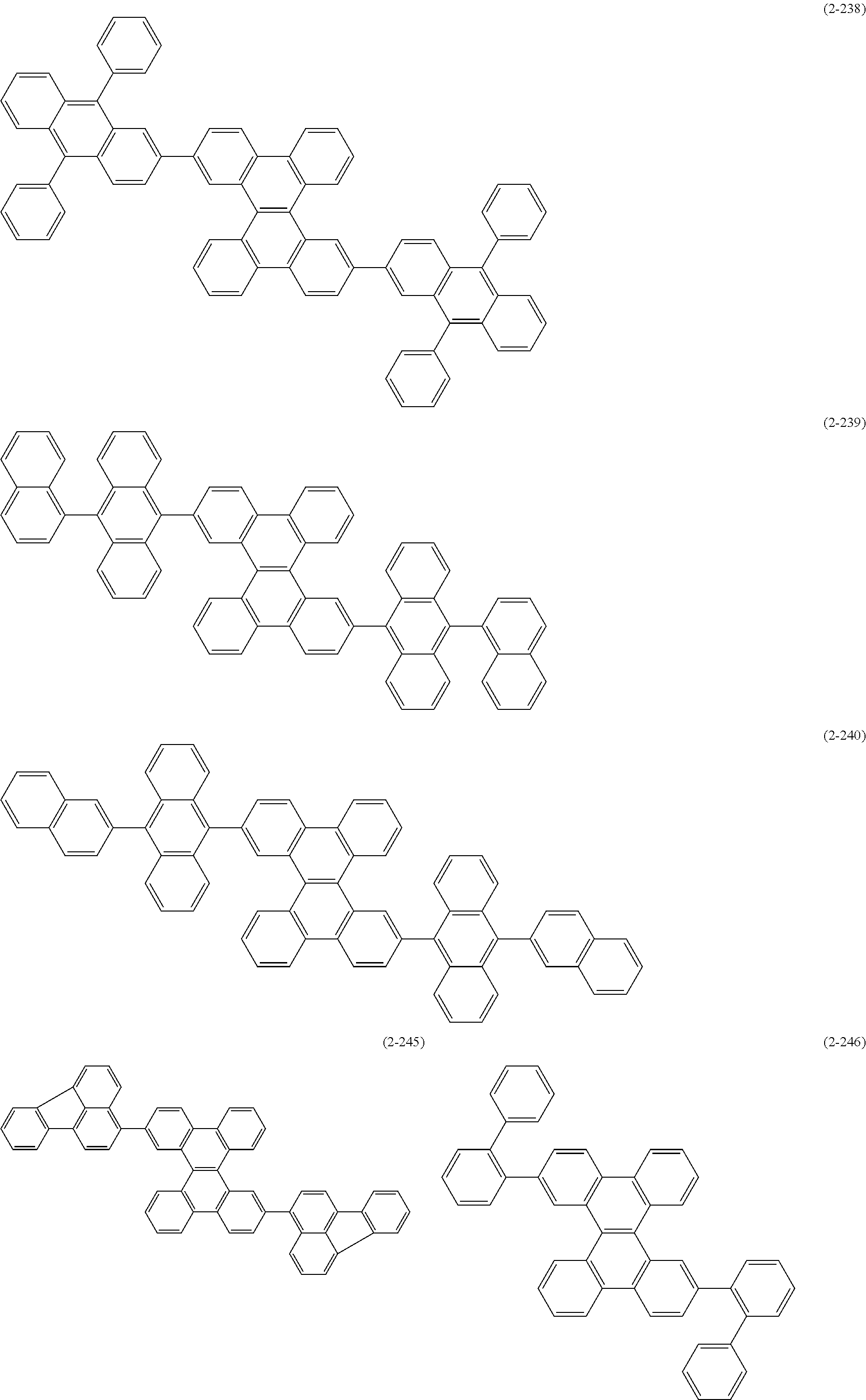

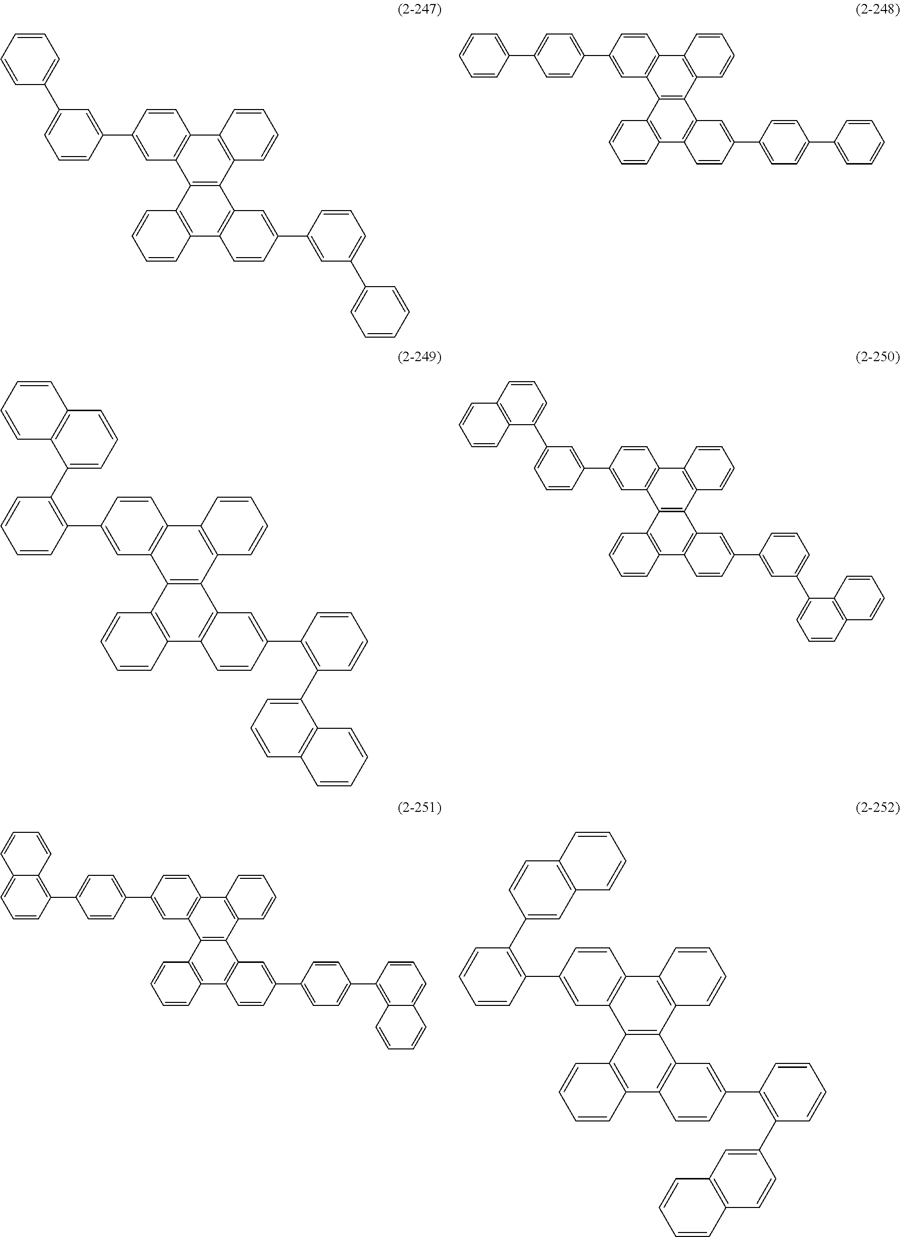

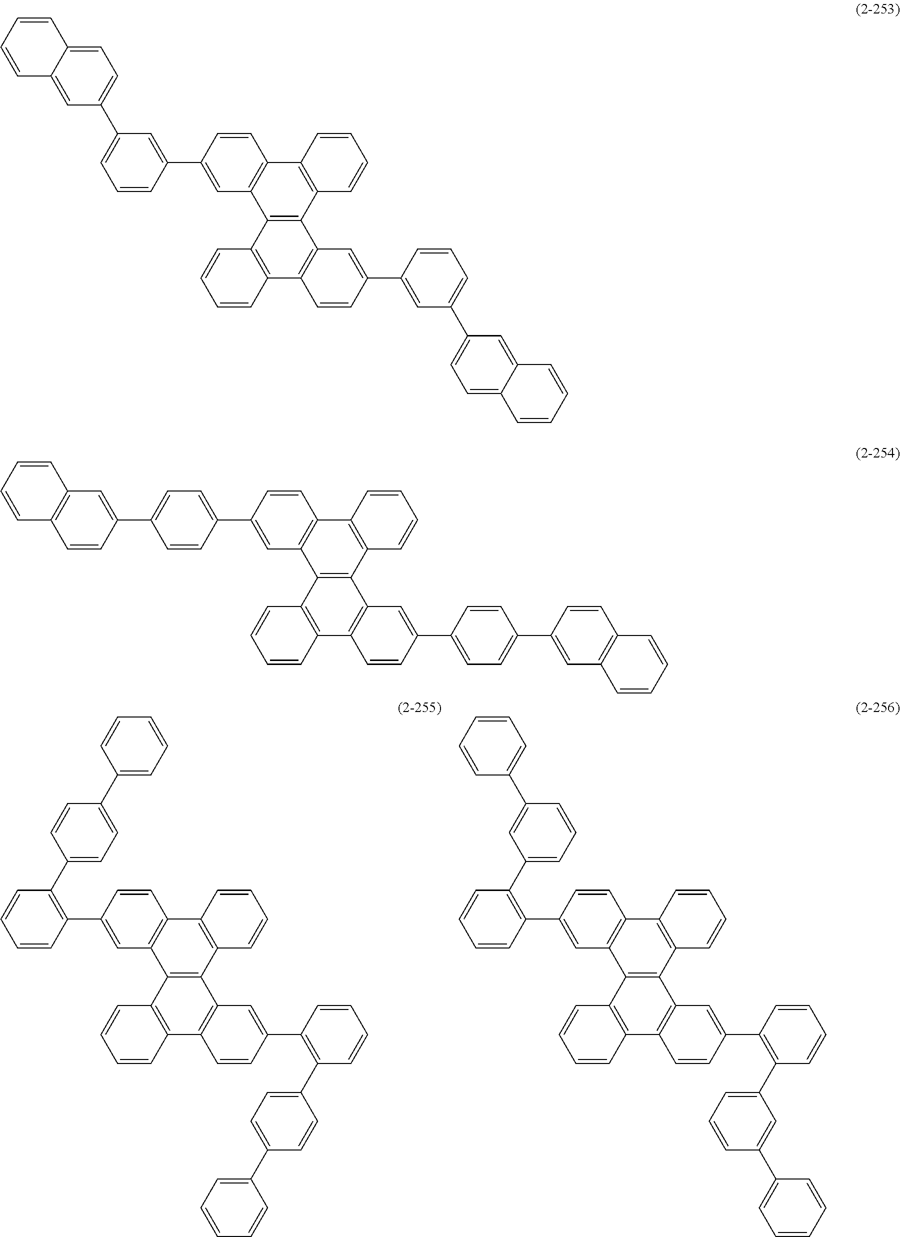

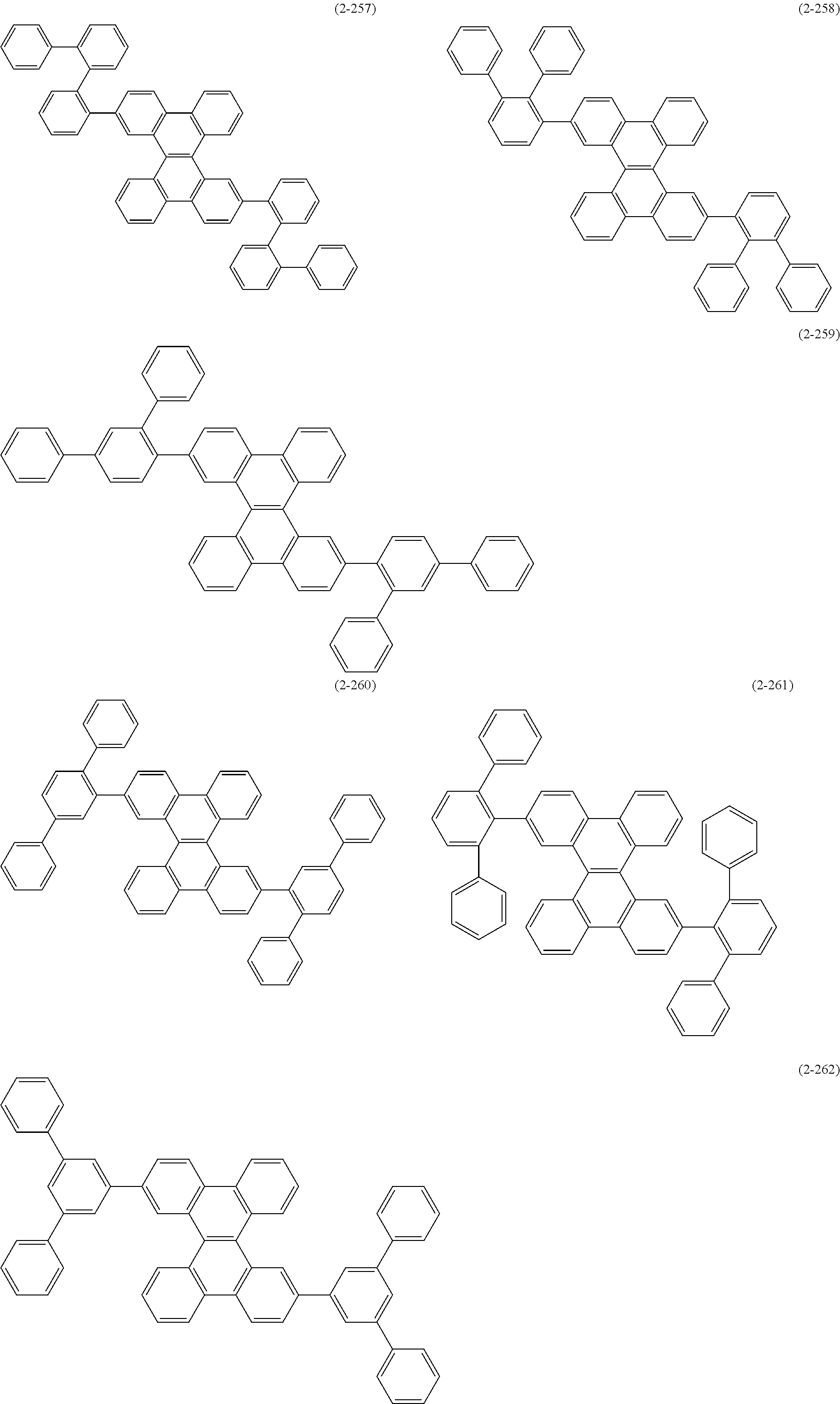

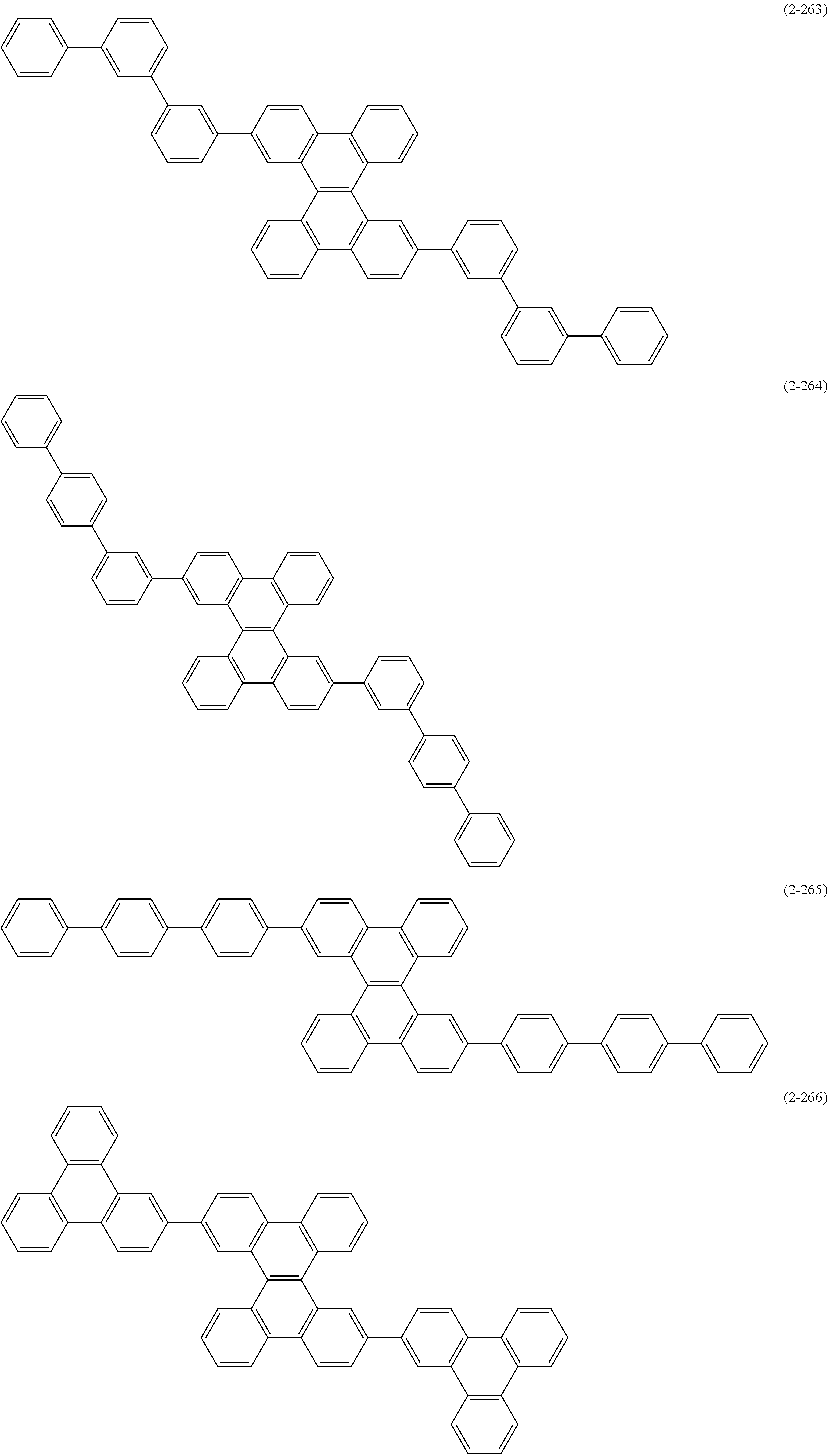

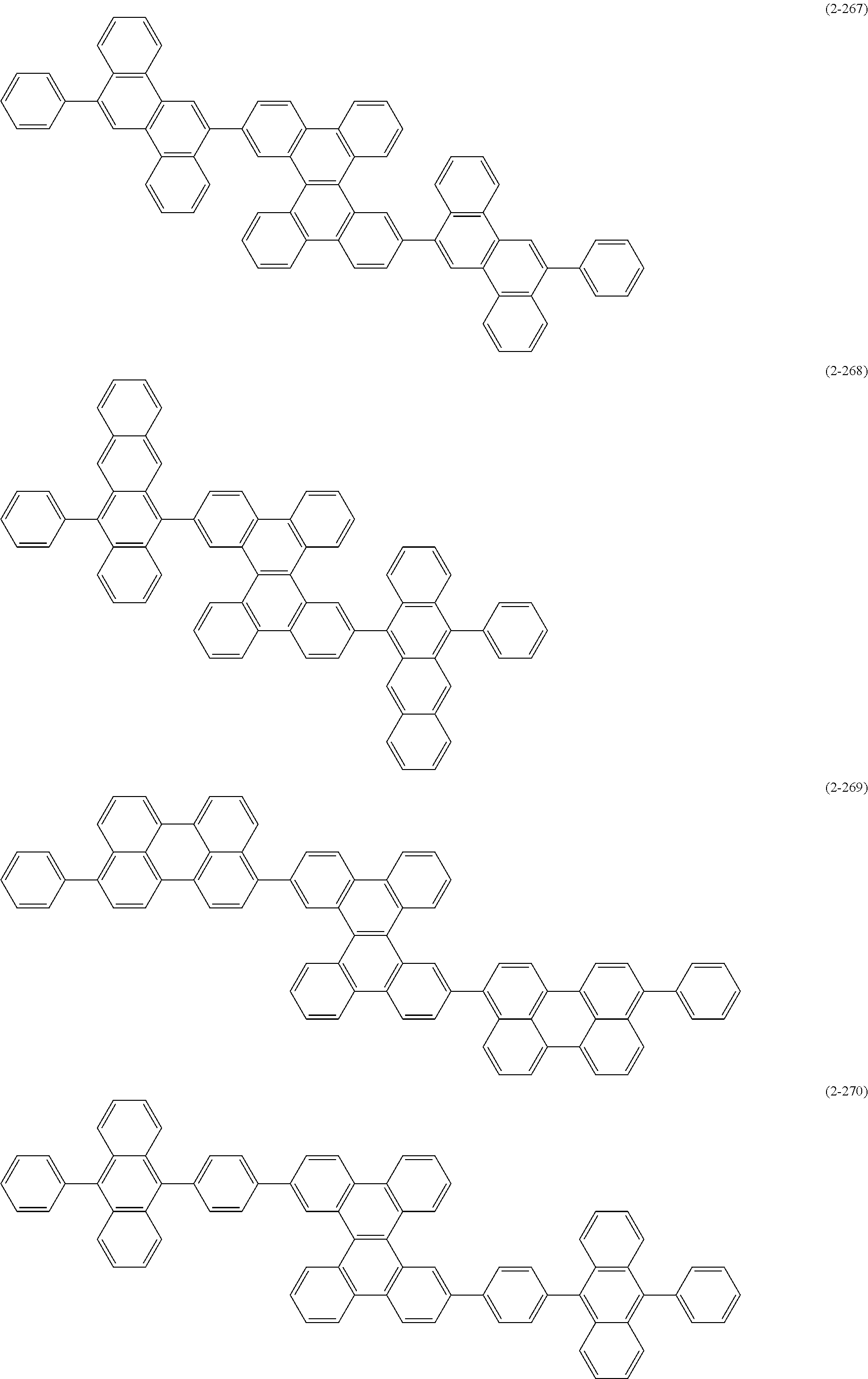

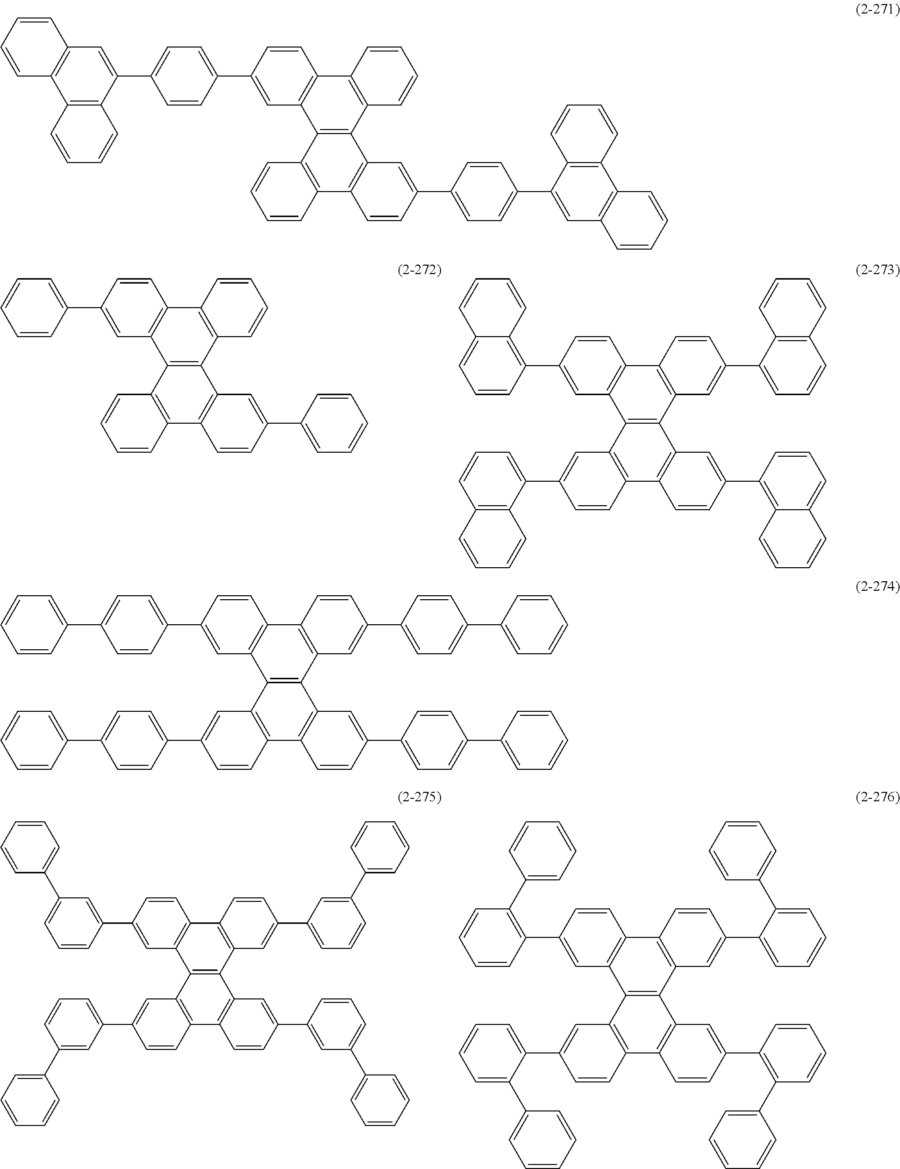

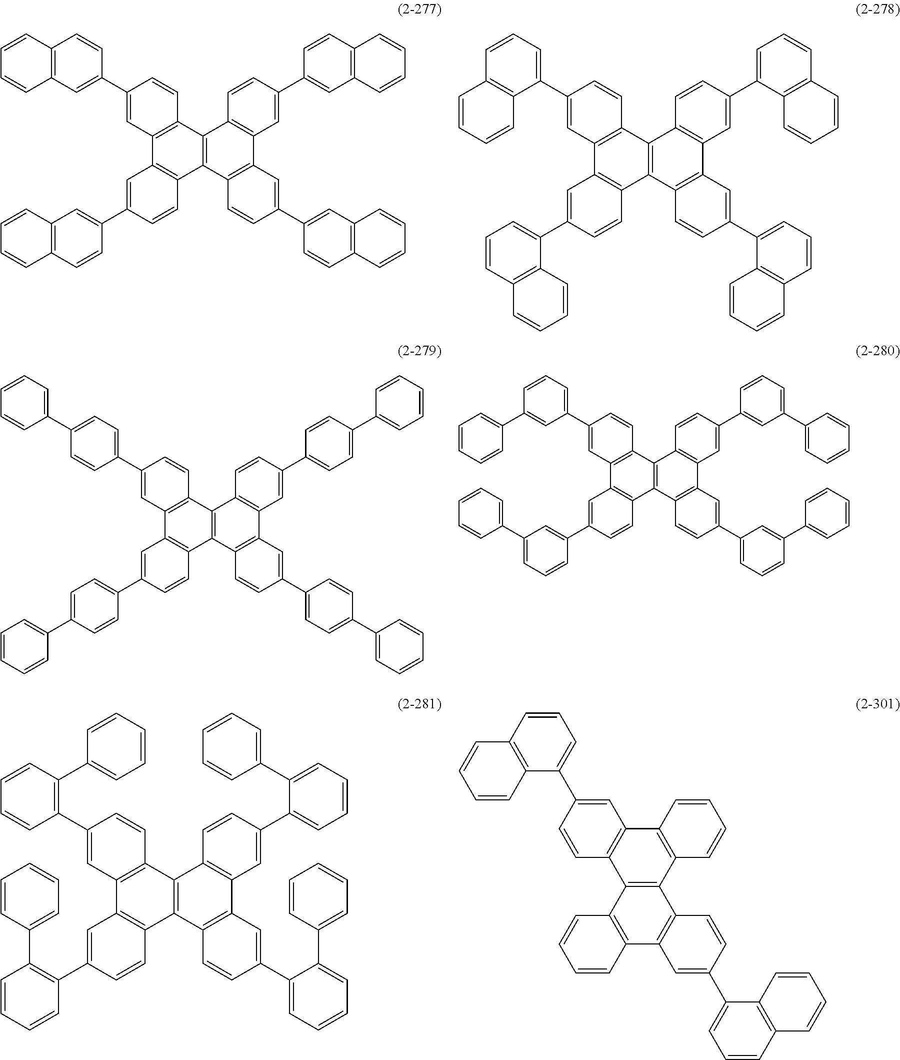

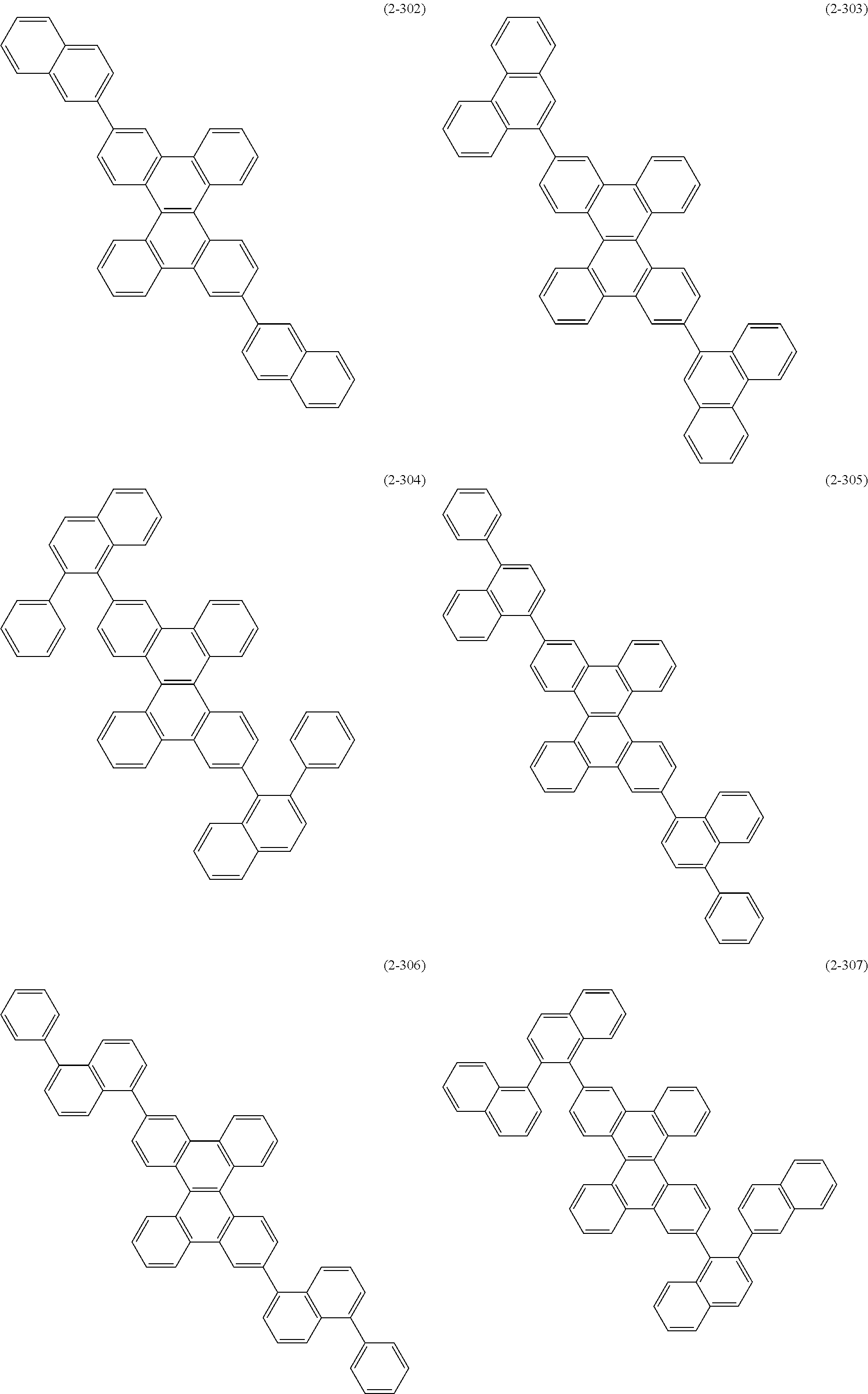

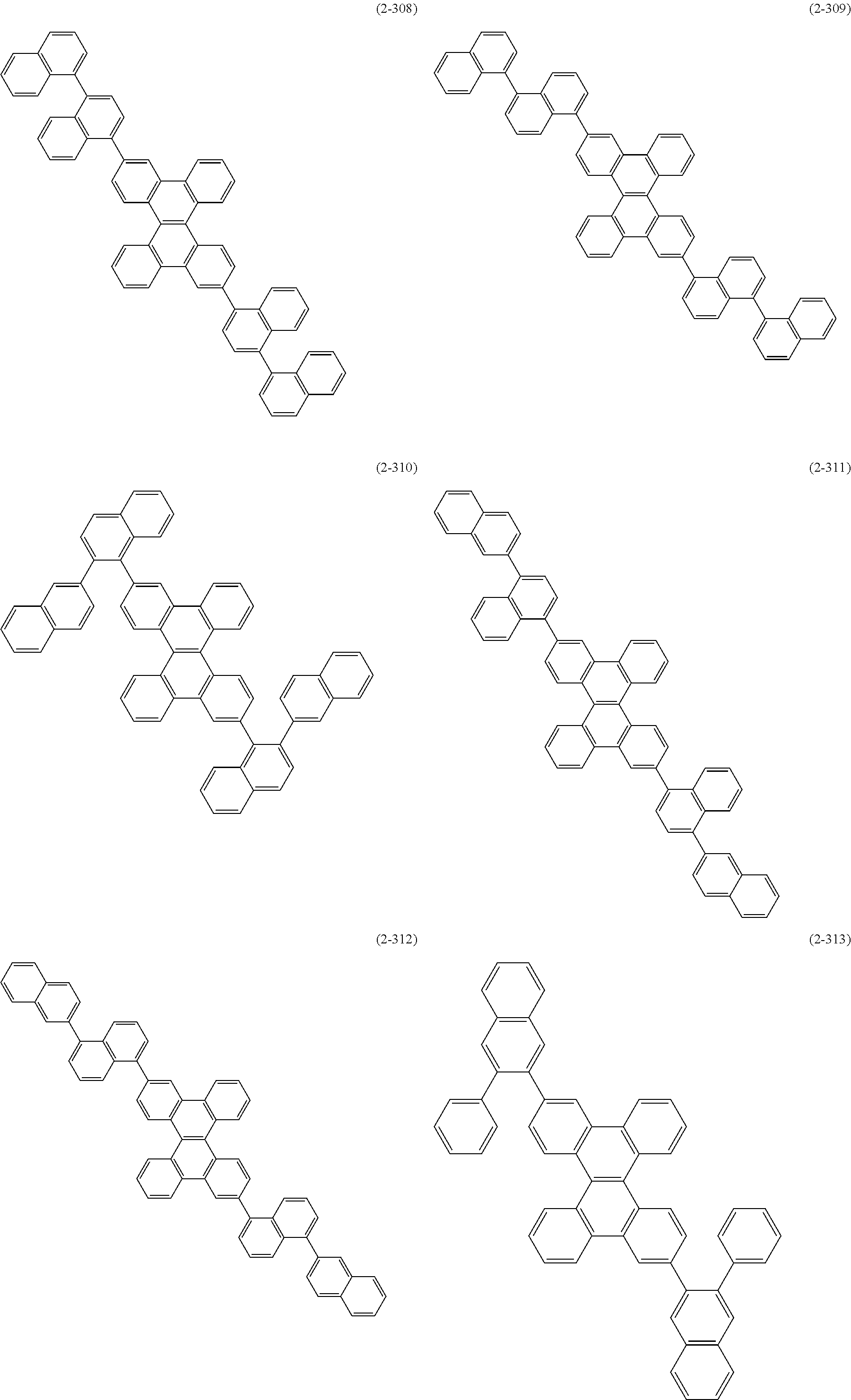

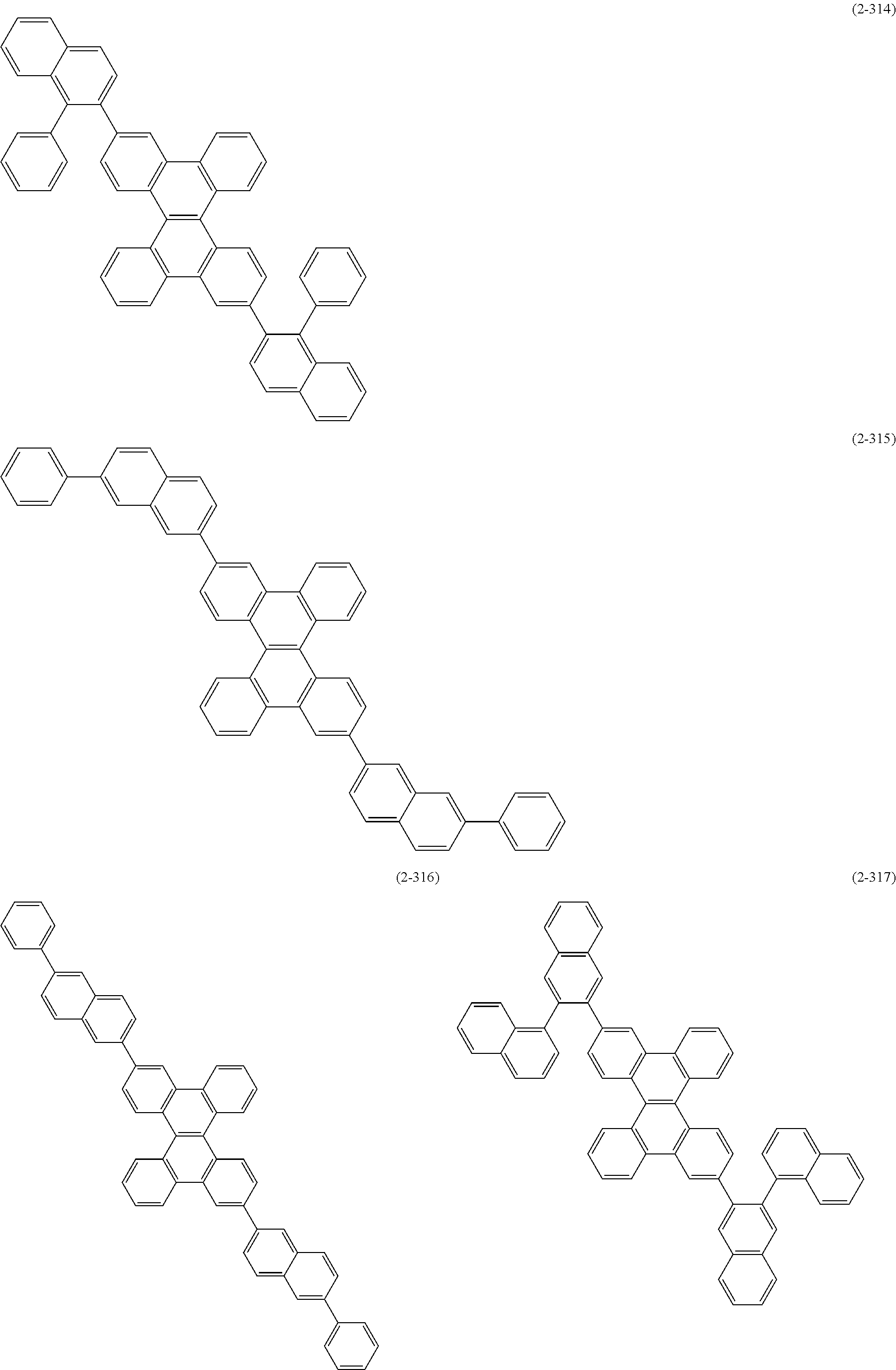

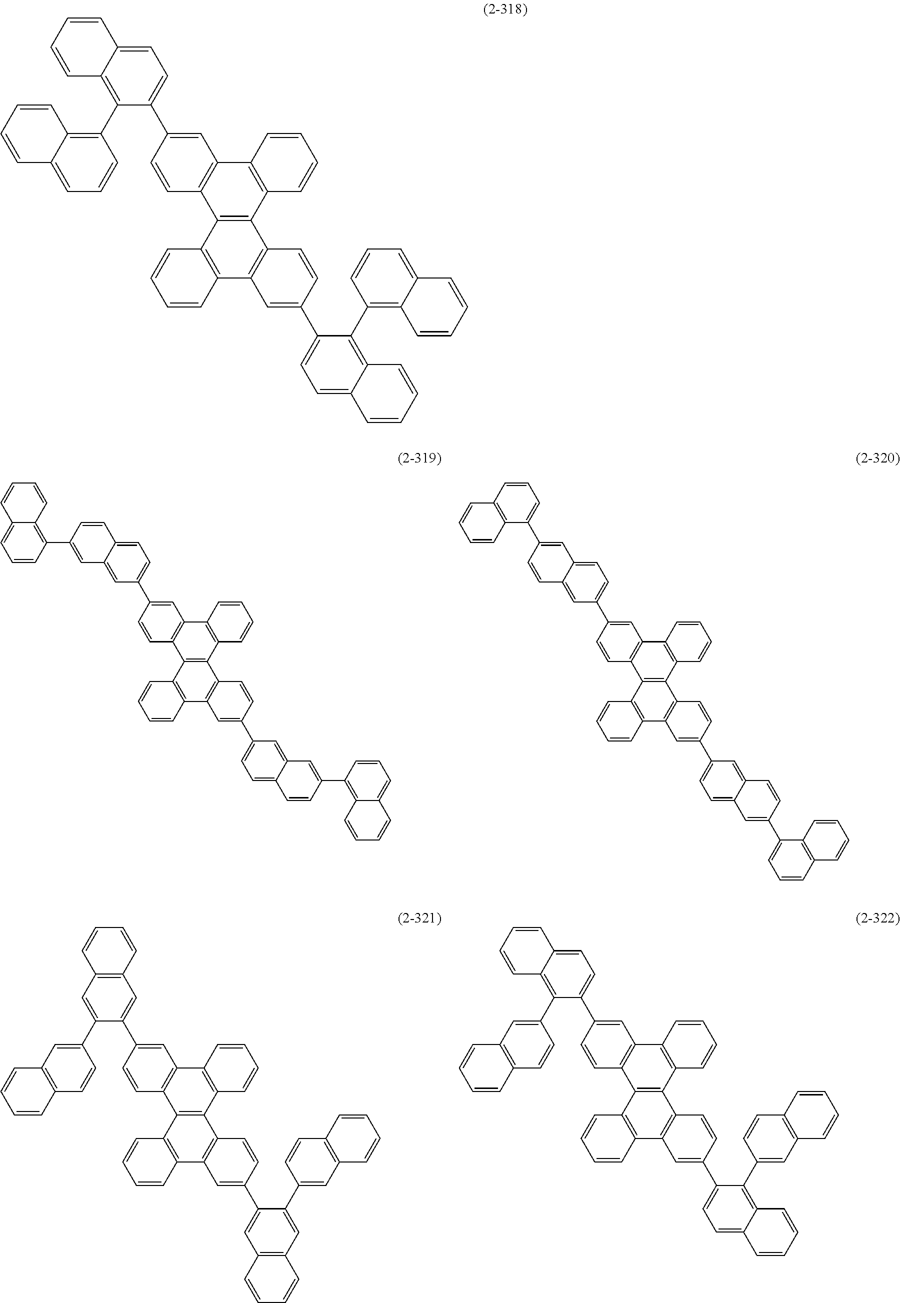

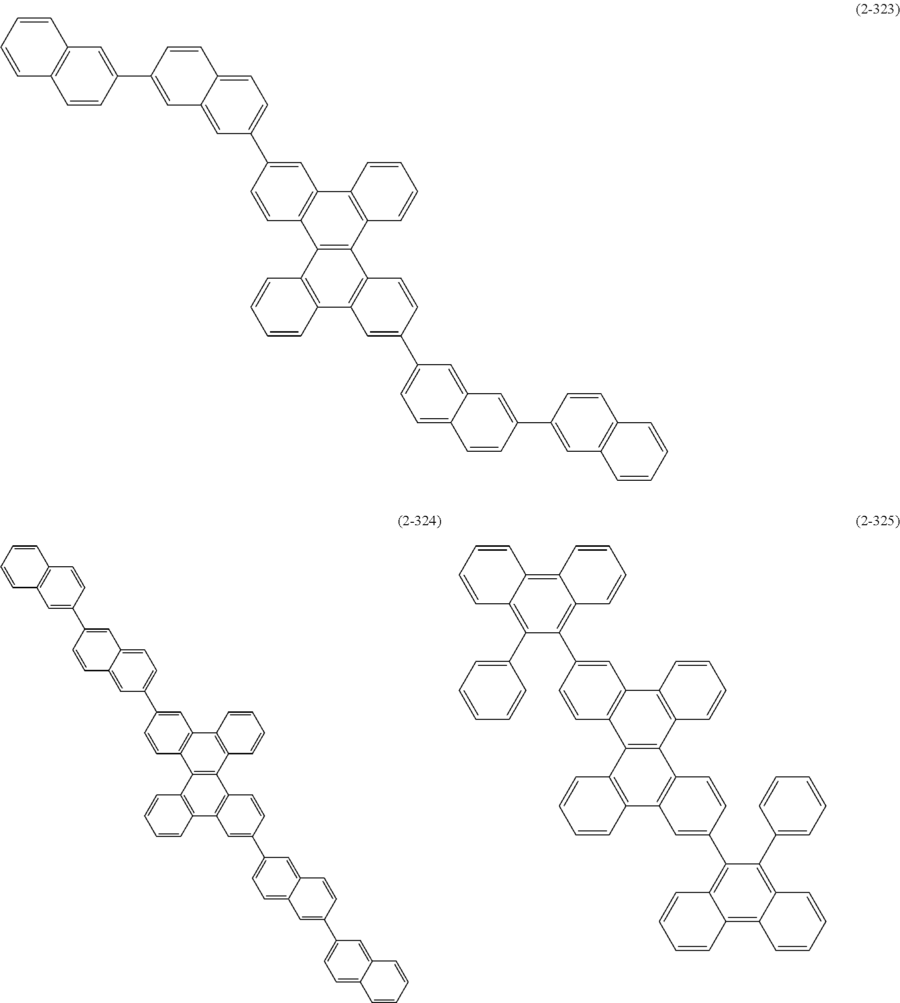

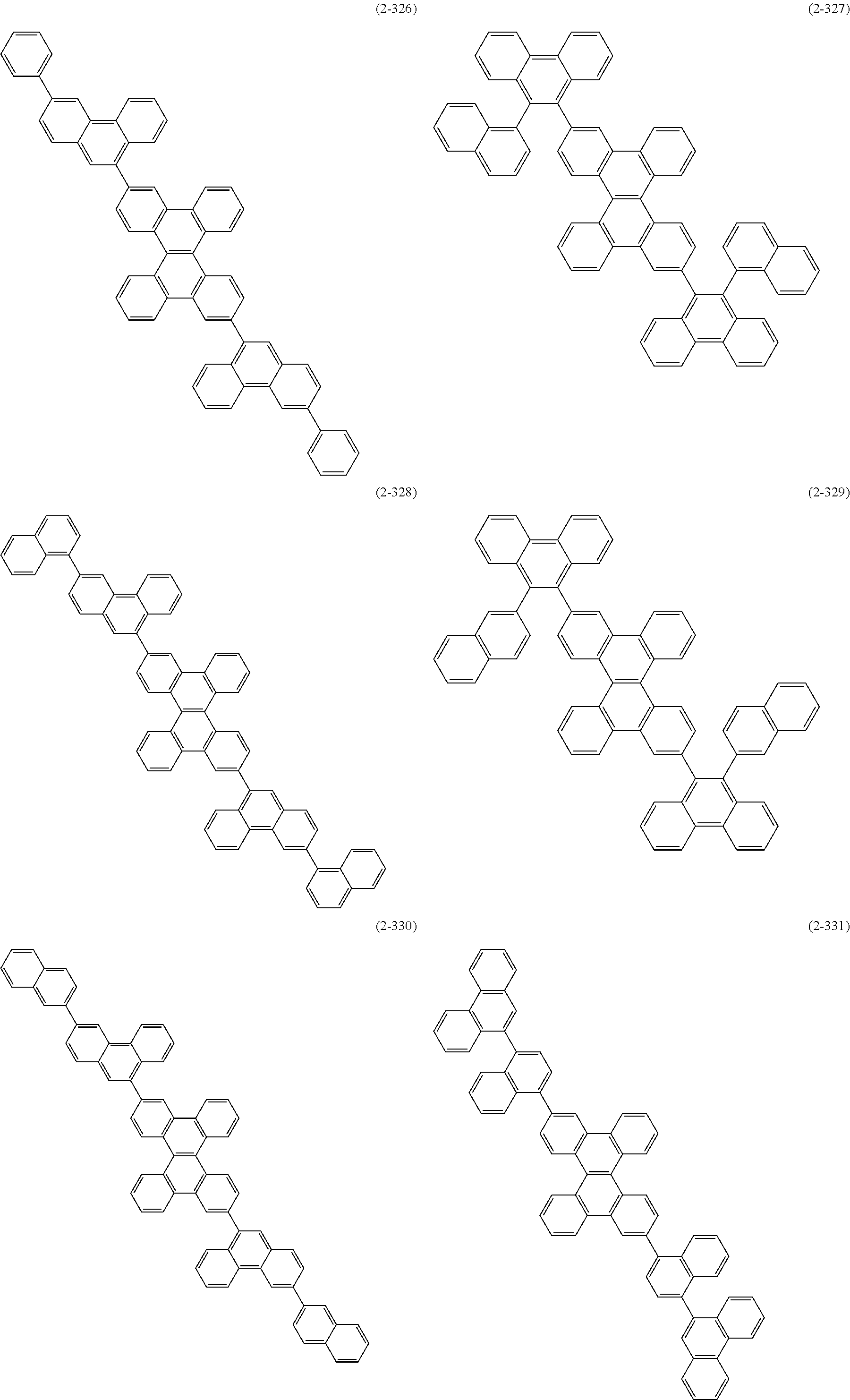

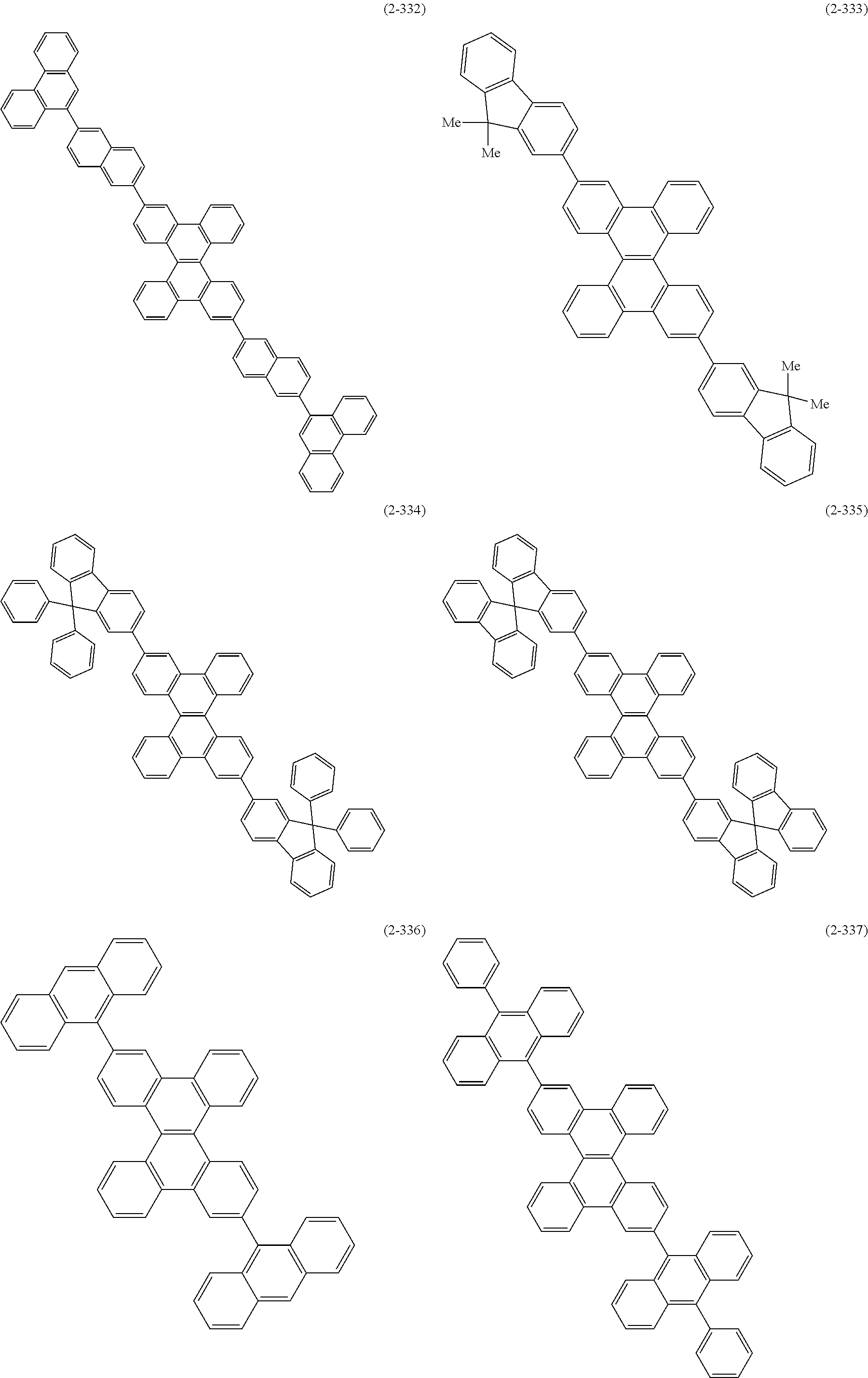

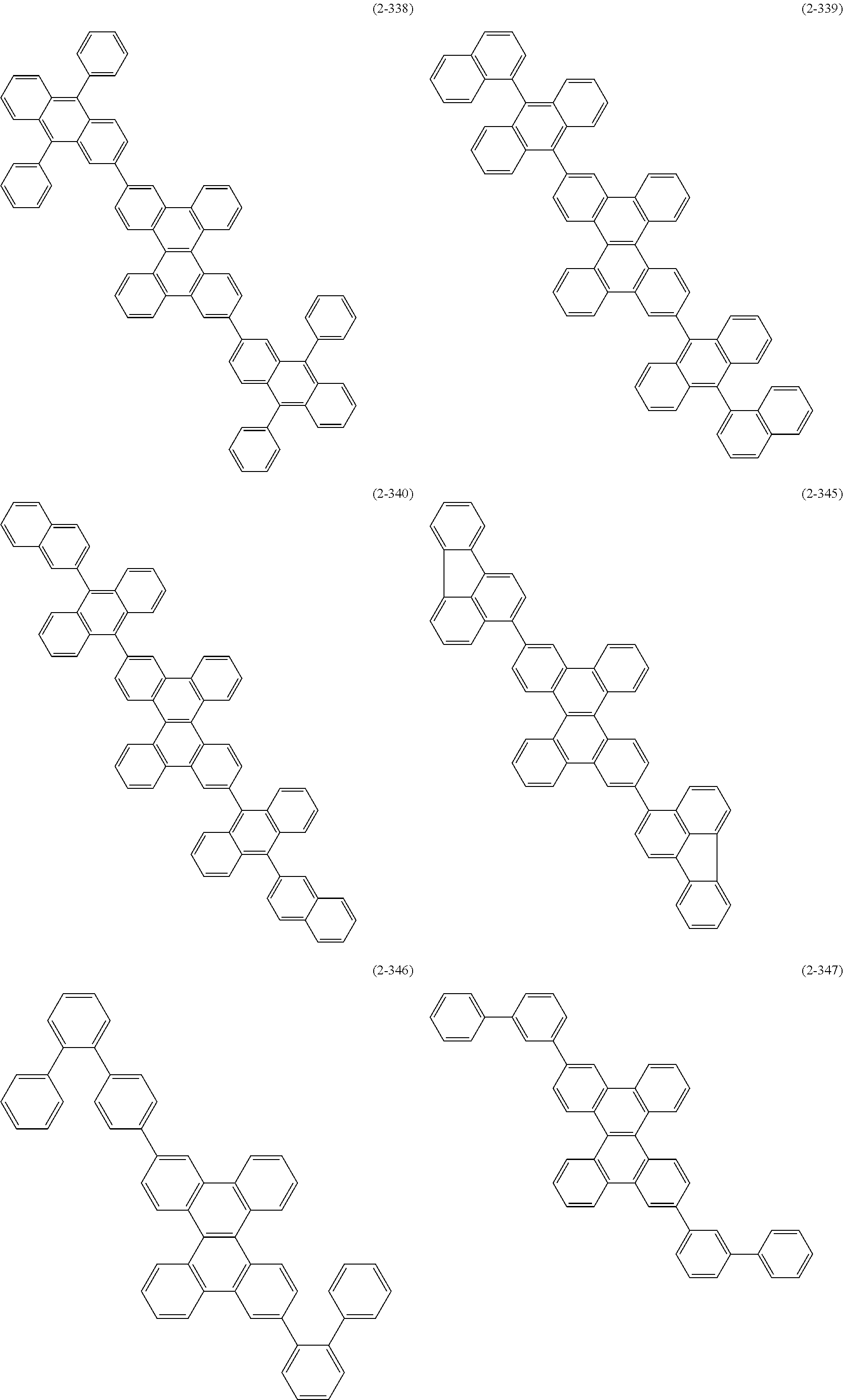

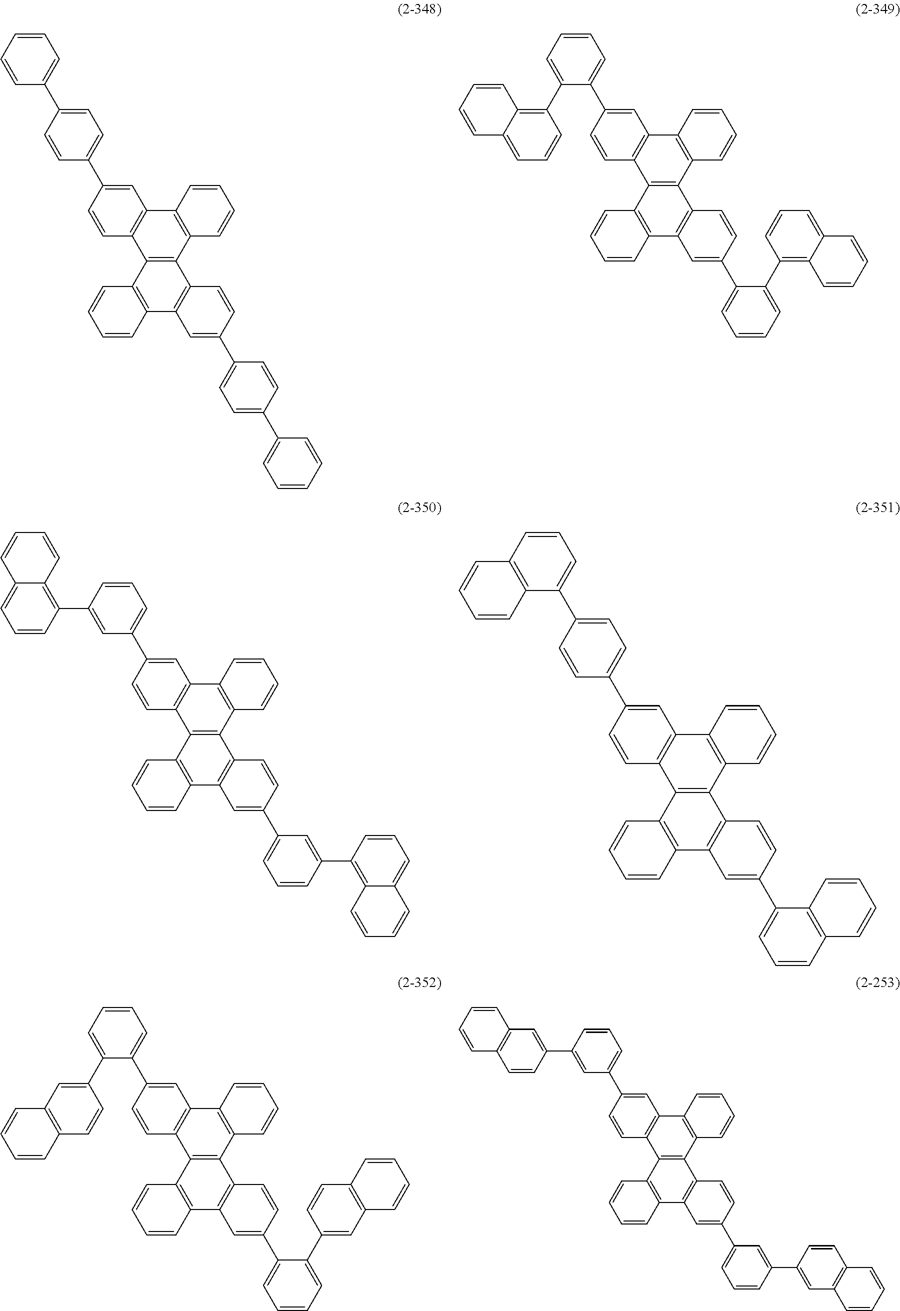

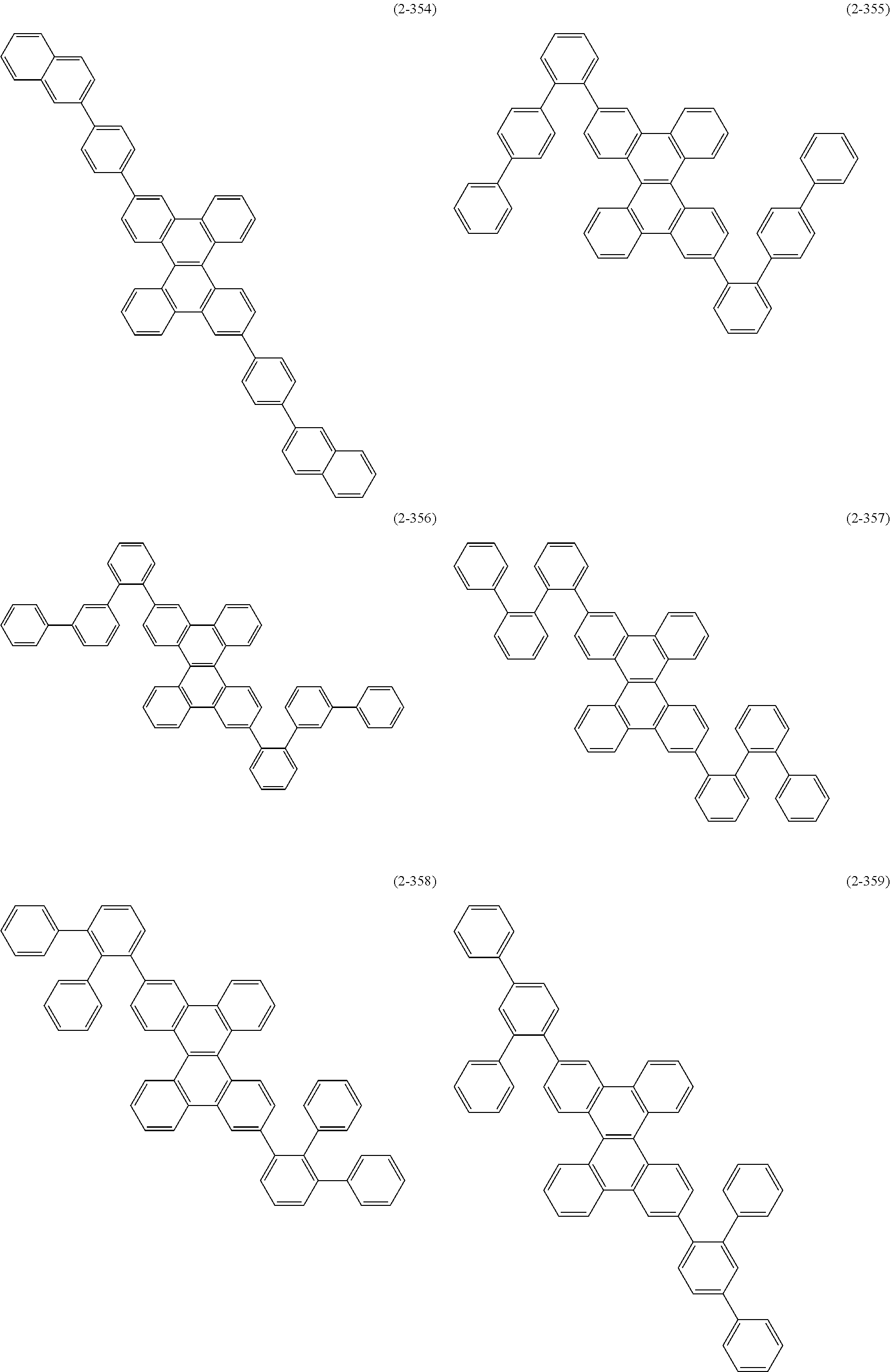

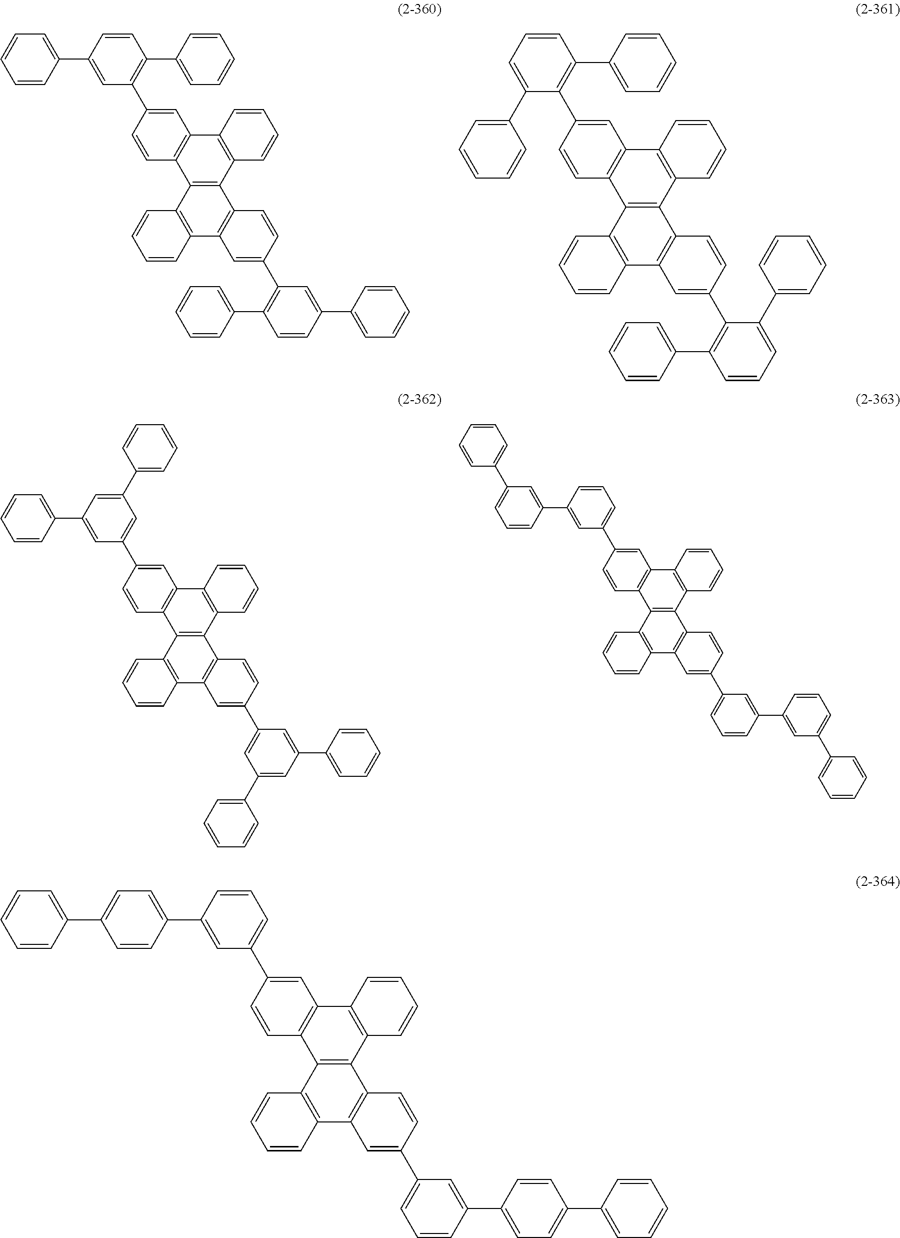

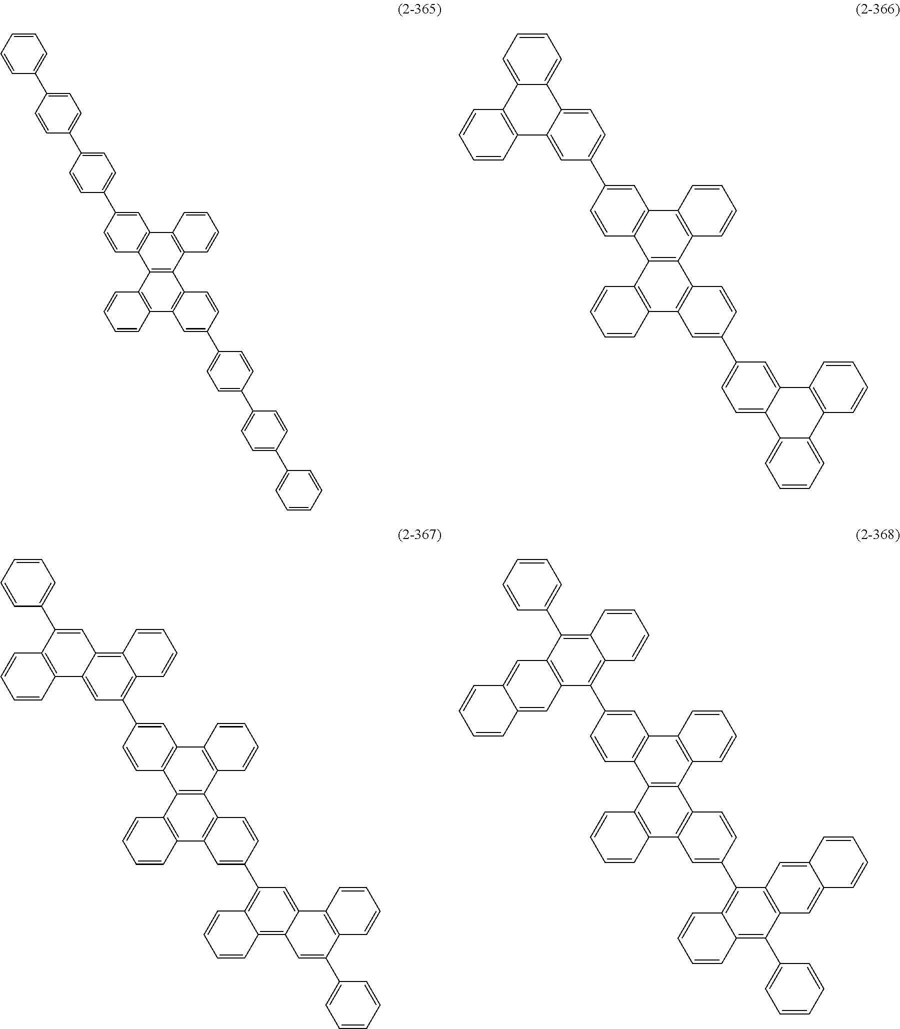

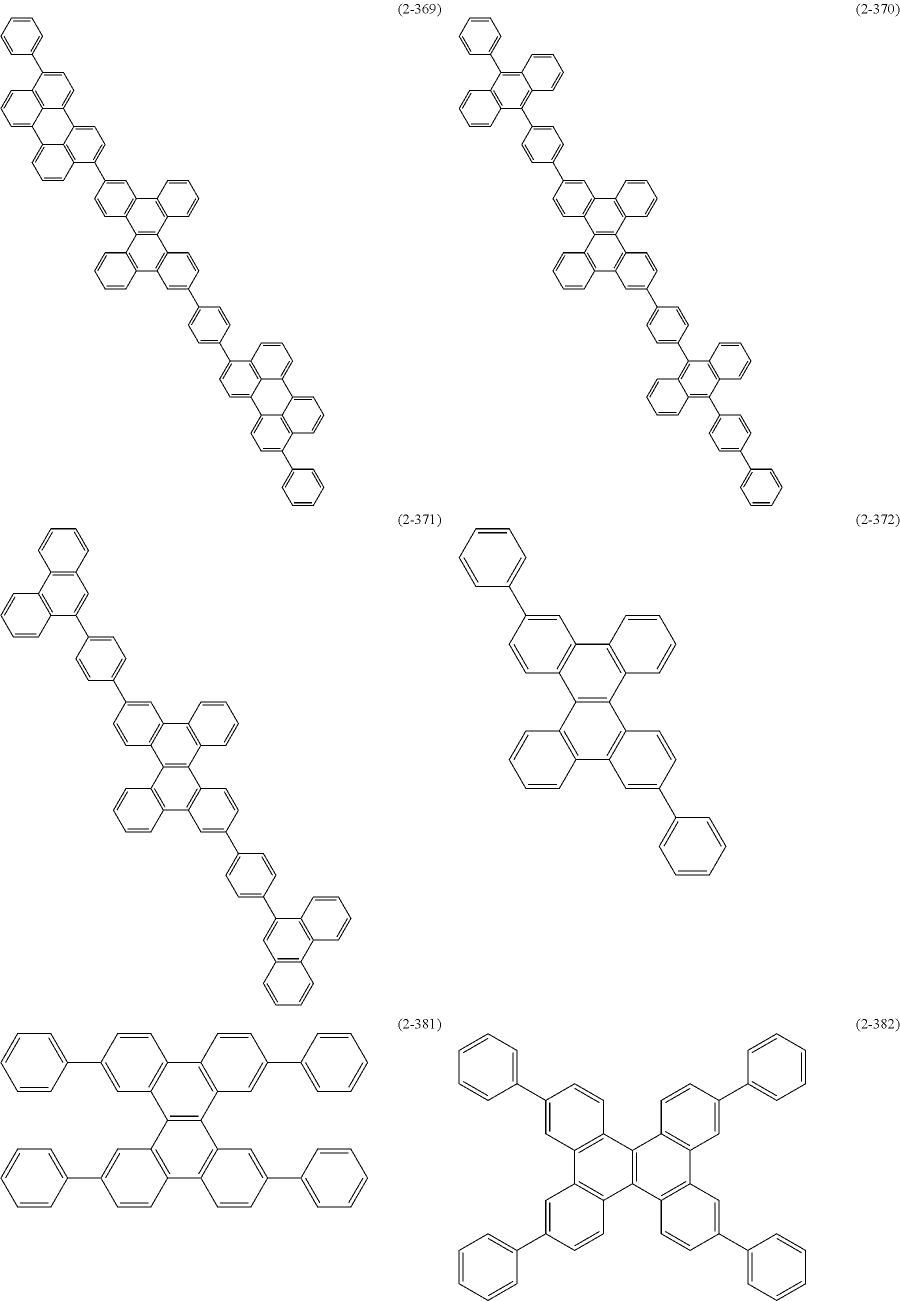

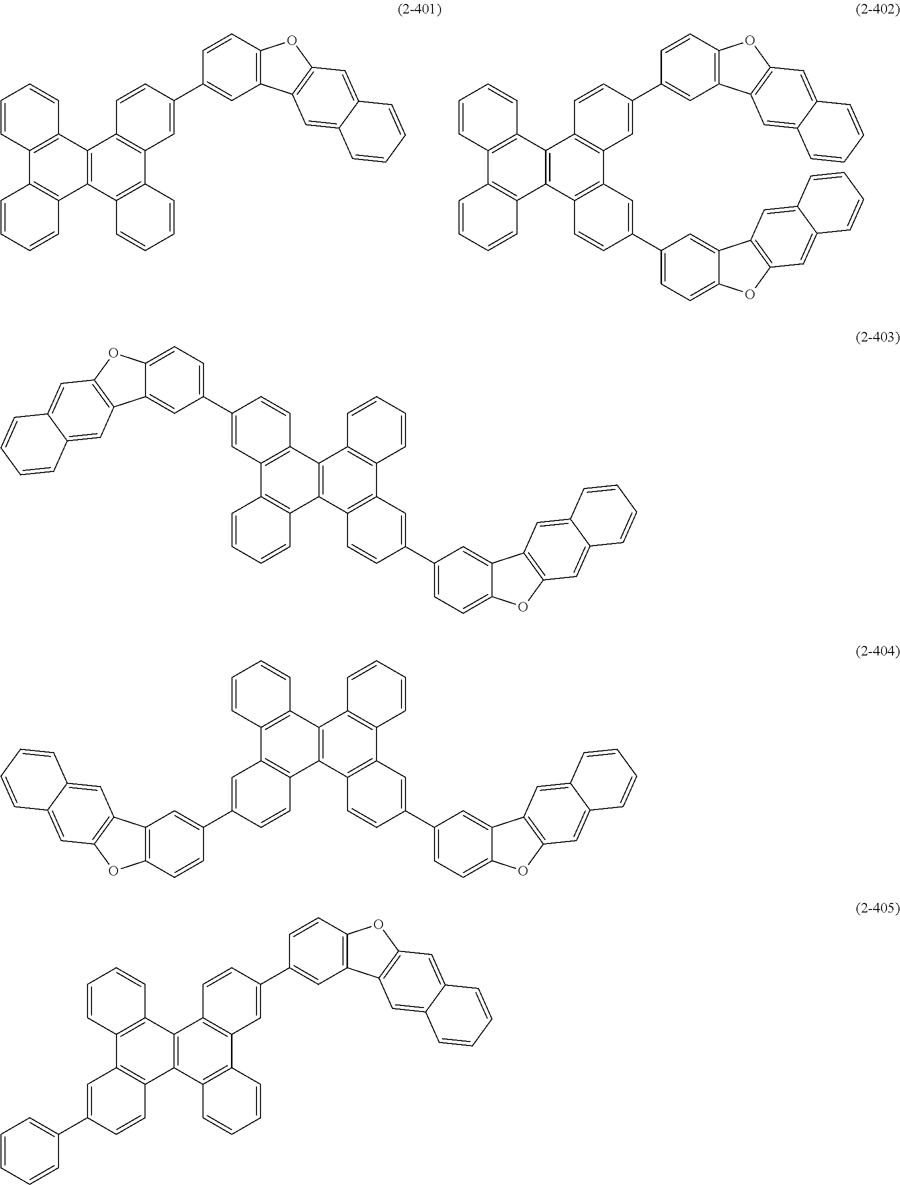 [SMILES: c1ccc2cc3c(cc2c1)oc1ccc(-c2ccc4c(c2)c2ccccc2c2c5ccccc5c5ccccc5c42)cc13 ; c1ccc2cc3c(cc2c1)oc1ccc(-c2ccc4c(c2)c2cc(-c5ccc6oc7cc8ccccc8cc7c6c5)ccc2c2c5ccccc5c5ccccc5c42)cc13 ; c1ccc2cc3c(cc2c1)oc1ccc(-c2ccc4c(c2)c2ccccc2c2c5ccc(-c6ccc7oc8cc9ccccc9cc8c7c6)cc5c5ccccc5c42)cc13 ; c1ccc2cc3c(cc2c1)oc1ccc(-c2ccc4c(c2)c2ccccc2c2c5ccccc5c5cc(-c6ccc7oc8cc9ccccc9cc8c7c6)ccc5c42)cc13 ; c1ccc(-c2ccc3c(c2)c2ccccc2c2c4ccc(-c5ccc6oc7cc8ccccc8cc7c6c5)cc4c4ccccc4c32)cc1]

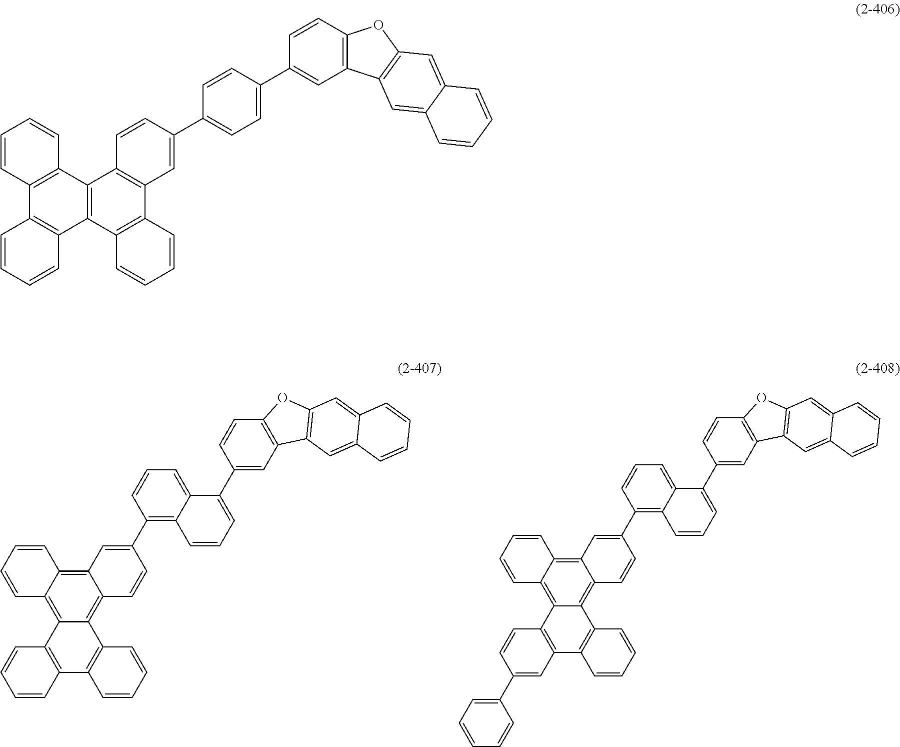 [SMILES: c1ccc2cc3c(cc2c1)oc1ccc(-c2ccc(-c4ccc5c(c4)c4ccccc4c4c6ccccc6c6ccccc6c54)cc2)cc13 ; c1ccc2cc3c(cc2c1)oc1ccc(-c2cccc4c(-c5ccc6c(c5)c5ccccc5c5c7ccccc7c7ccccc7c65)cccc24)cc13 ; c1ccc(-c2ccc3c(c2)c2ccccc2c2c4ccc(-c5cccc6c(-c7ccc8oc9cc%10ccccc%10cc9c8c7)cccc56)cc4c4ccccc4c32)cc1]

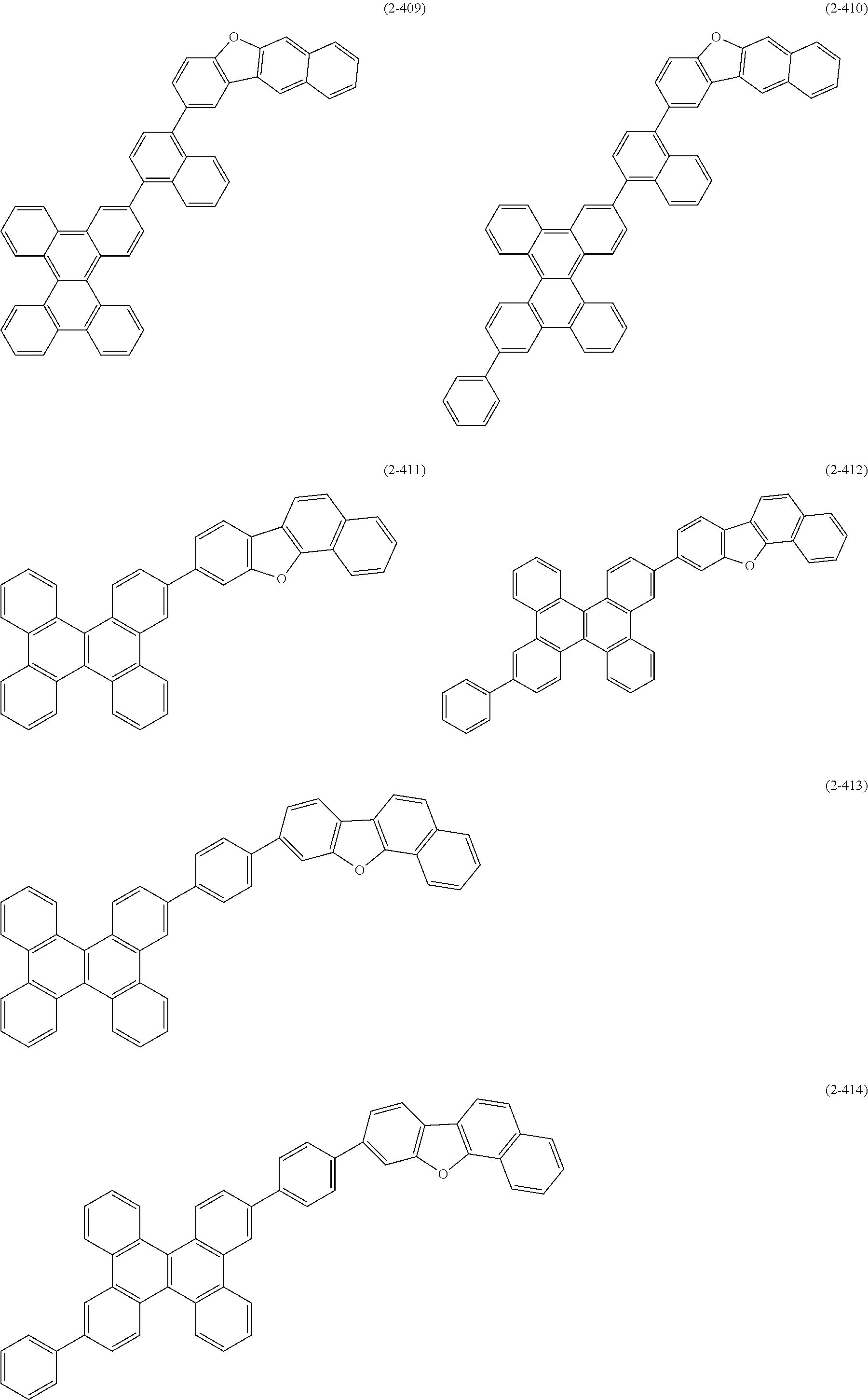

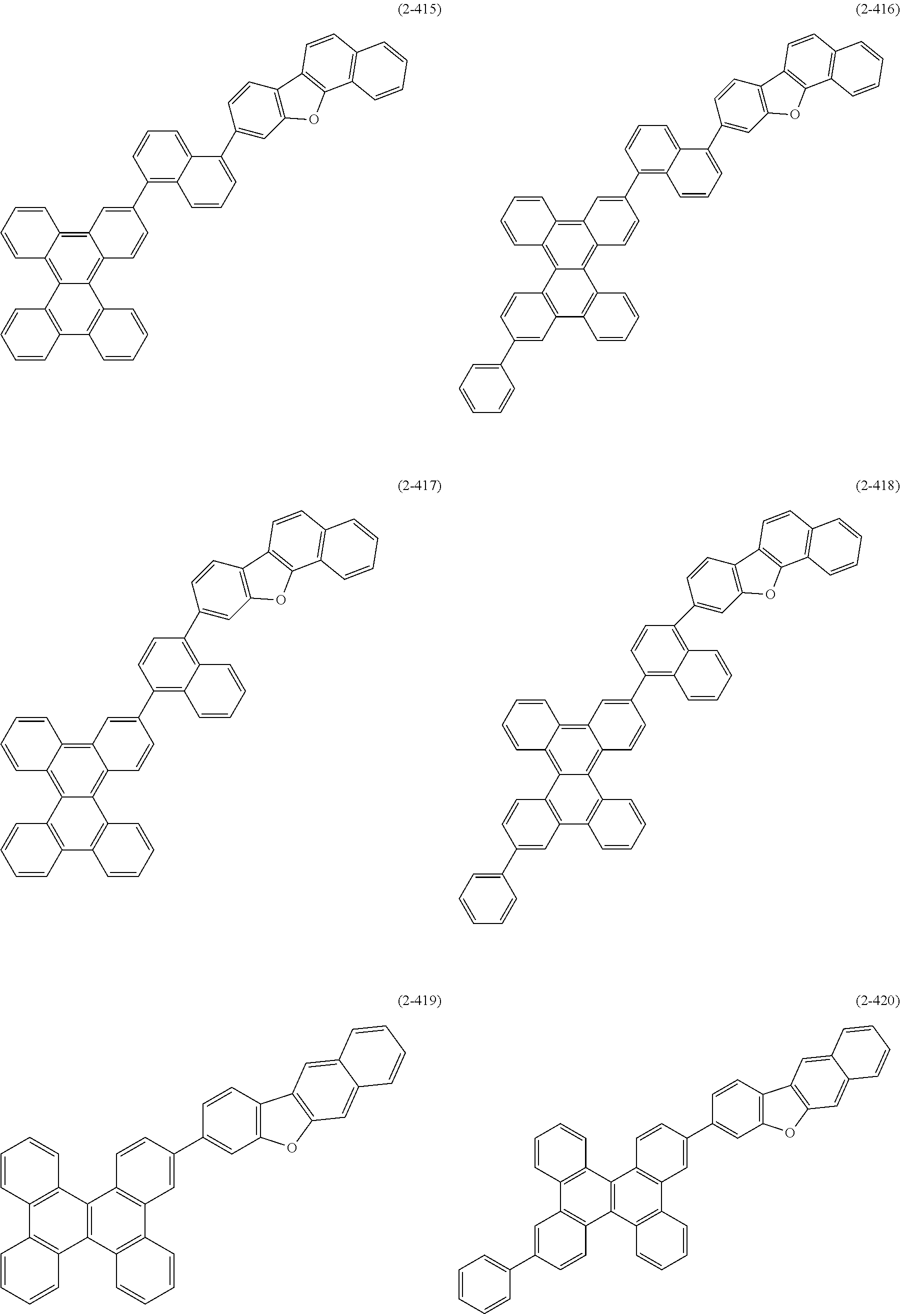

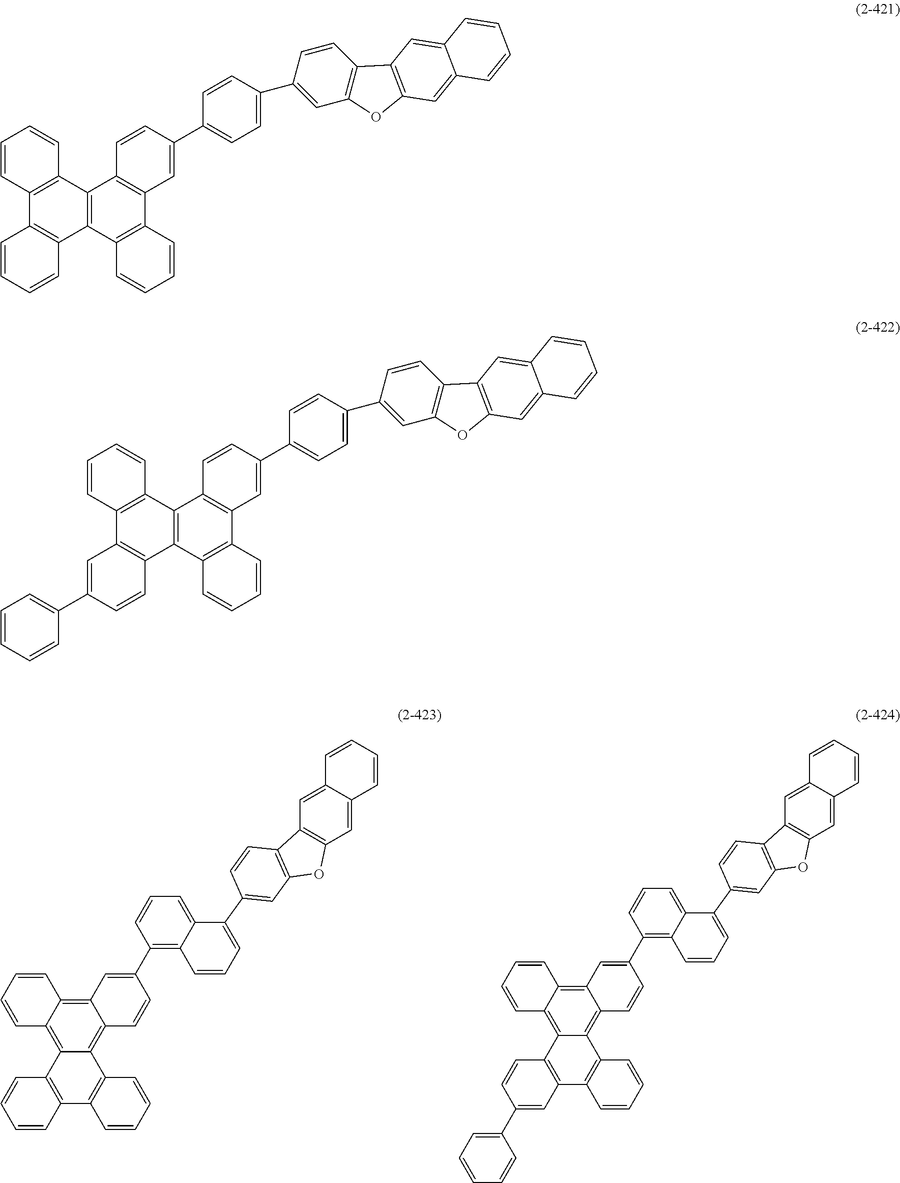 [SMILES: c1ccc2cc3c(cc2c1)oc1cc(-c2ccc(-c4ccc5c(c4)c4ccccc4c4c6ccccc6c6ccccc6c54)cc2)ccc13 ; c1ccc(-c2ccc3c(c2)c2ccccc2c2c4ccc(-c5ccc(-c6ccc7c(c6)oc6cc8ccccc8cc67)cc5)cc4c4ccccc4c32)cc1 ; c1ccc2cc3c(cc2c1)oc1cc(-c2cccc4c(-c5ccc6c(c5)c5ccccc5c5c7ccccc7c7ccccc7c65)cccc24)ccc13 ; c1ccc(-c2ccc3c(c2)c2ccccc2c2c4ccc(-c5cccc6c(-c7ccc8c(c7)oc7cc9ccccc9cc78)cccc56)cc4c4ccccc4c32)cc1]

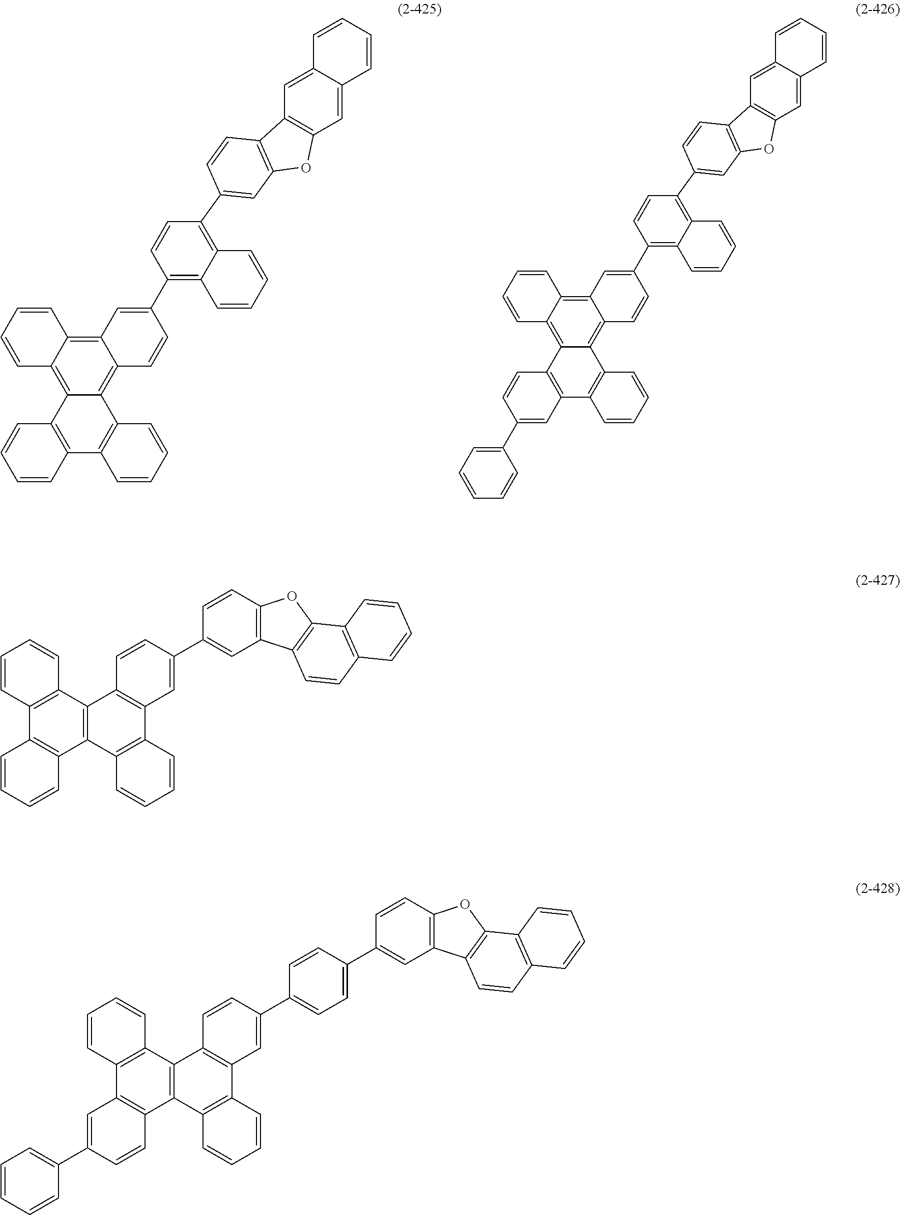 [SMILES: c1ccc2cc3c(cc2c1)oc1cc(-c2ccc(-c4ccc5c(c4)c4ccccc4c4c6ccccc6c6ccccc6c54)c4ccccc24)ccc13 ; c1ccc(-c2ccc3c(c2)c2ccccc2c2c4ccc(-c5ccc(-c6ccc7c(c6)oc6cc8ccccc8cc67)c6ccccc56)cc4c4ccccc4c32)cc1 ; c1ccc2c(c1)ccc1c3cc(-c4ccc5c(c4)c4ccccc4c4c6ccccc6c6ccccc6c54)ccc3oc21 ; c1ccc(-c2ccc3c(c2)c2ccccc2c2c4ccc(-c5ccc(-c6ccc7oc8c9ccccc9ccc8c7c6)cc5)cc4c4ccccc4c32)cc1]

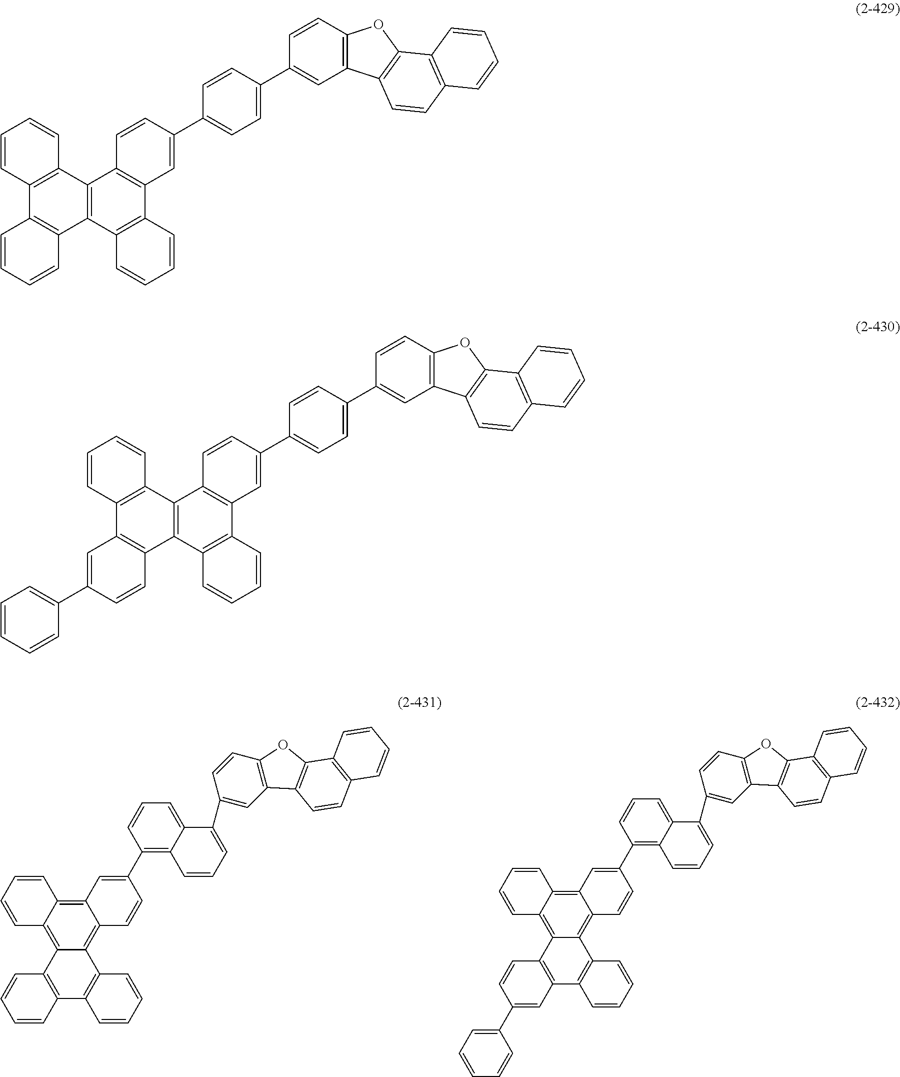 [SMILES: c1ccc2c(c1)ccc1c3cc(-c4ccc(-c5ccc6c(c5)c5ccccc5c5c7ccccc7c7ccccc7c65)cc4)ccc3oc21 ; c1ccc(-c2ccc3c(c2)c2ccccc2c2c4ccc(-c5ccc(-c6ccc7oc8c9ccccc9ccc8c7c6)cc5)cc4c4ccccc4c32)cc1 ; c1ccc2c(c1)ccc1c3cc(-c4cccc5c(-c6ccc7c(c6)c6ccccc6c6c8ccccc8c8ccccc8c76)cccc45)ccc3oc21 ; c1ccc(-c2ccc3c(c2)c2ccccc2c2c4ccc(-c5cccc6c(-c7ccc8oc9c%10ccccc%10ccc9c8c7)cccc56)cc4c4ccccc4c32)cc1]

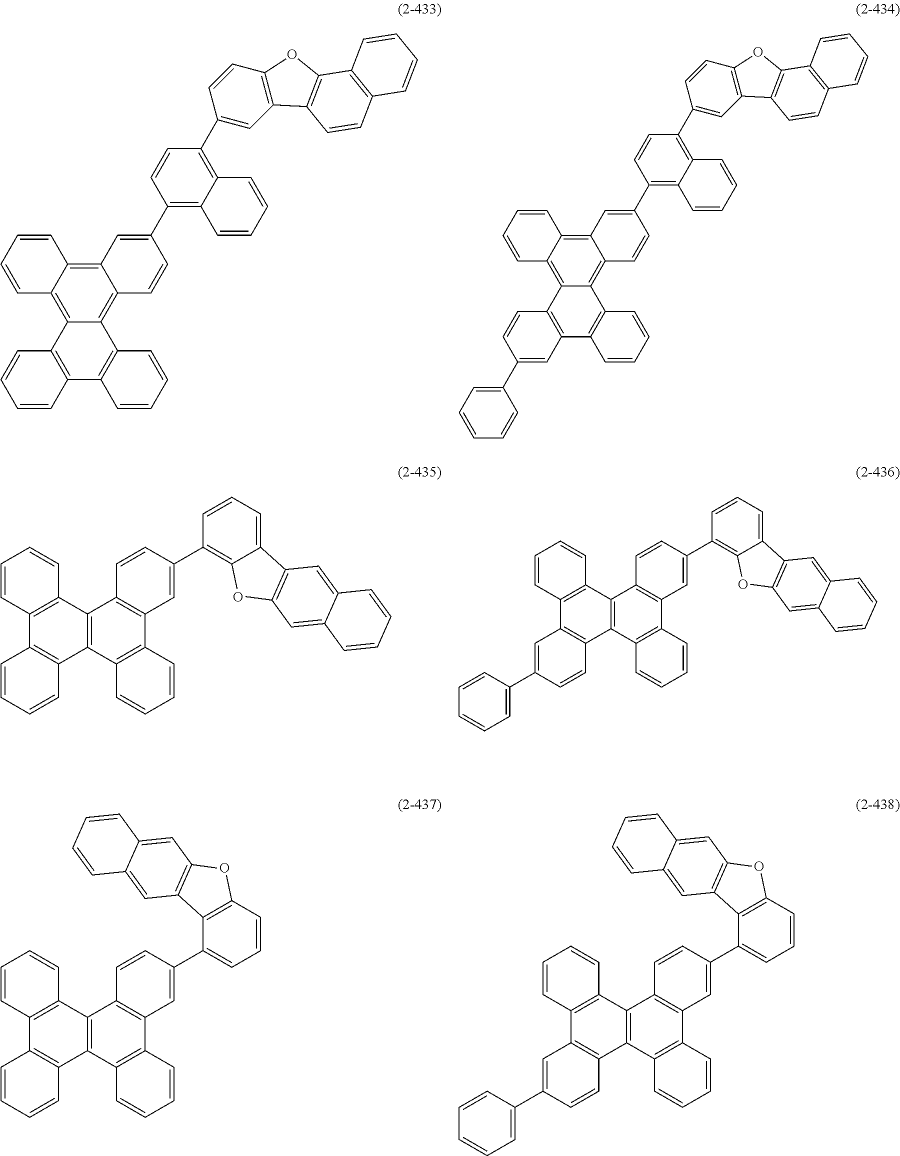 [SMILES: c1ccc2c(c1)ccc1c3cc(-c4ccc(-c5ccc6c(c5)c5ccccc5c5c7ccccc7c7ccccc7c65)c5ccccc45)ccc3oc21 ; c1ccc(-c2ccc3c(c2)c2ccccc2c2c4ccc(-c5ccc(-c6ccc7oc8c9ccccc9ccc8c7c6)c6ccccc56)cc4c4ccccc4c32)cc1 ; c1ccc2cc3c(cc2c1)oc1c(-c2ccc4c(c2)c2ccccc2c2c5ccccc5c5ccccc5c42)cccc13 ; c1ccc(-c2ccc3c(c2)c2ccccc2c2c4ccc(-c5cccc6c5oc5cc7ccccc7cc56)cc4c4ccccc4c32)cc1 ; c1ccc2cc3c(cc2c1)oc1cccc(-c2ccc4c(c2)c2ccccc2c2c5ccccc5c5ccccc5c42)c13 ; c1ccc(-c2ccc3c(c2)c2ccccc2c2c4ccc(-c5cccc6oc7cc8ccccc8cc7c56)cc4c4ccccc4c32)cc1]

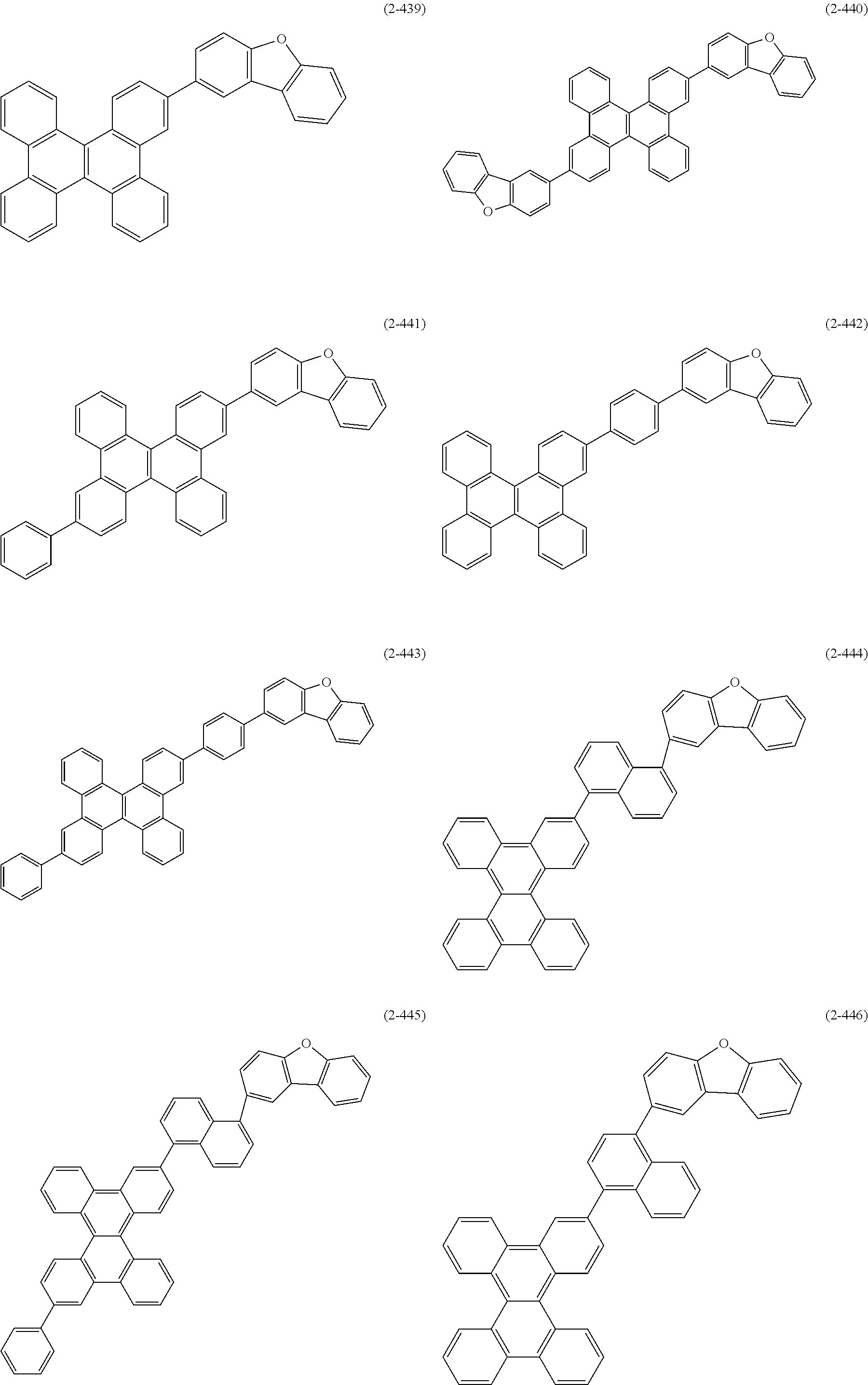

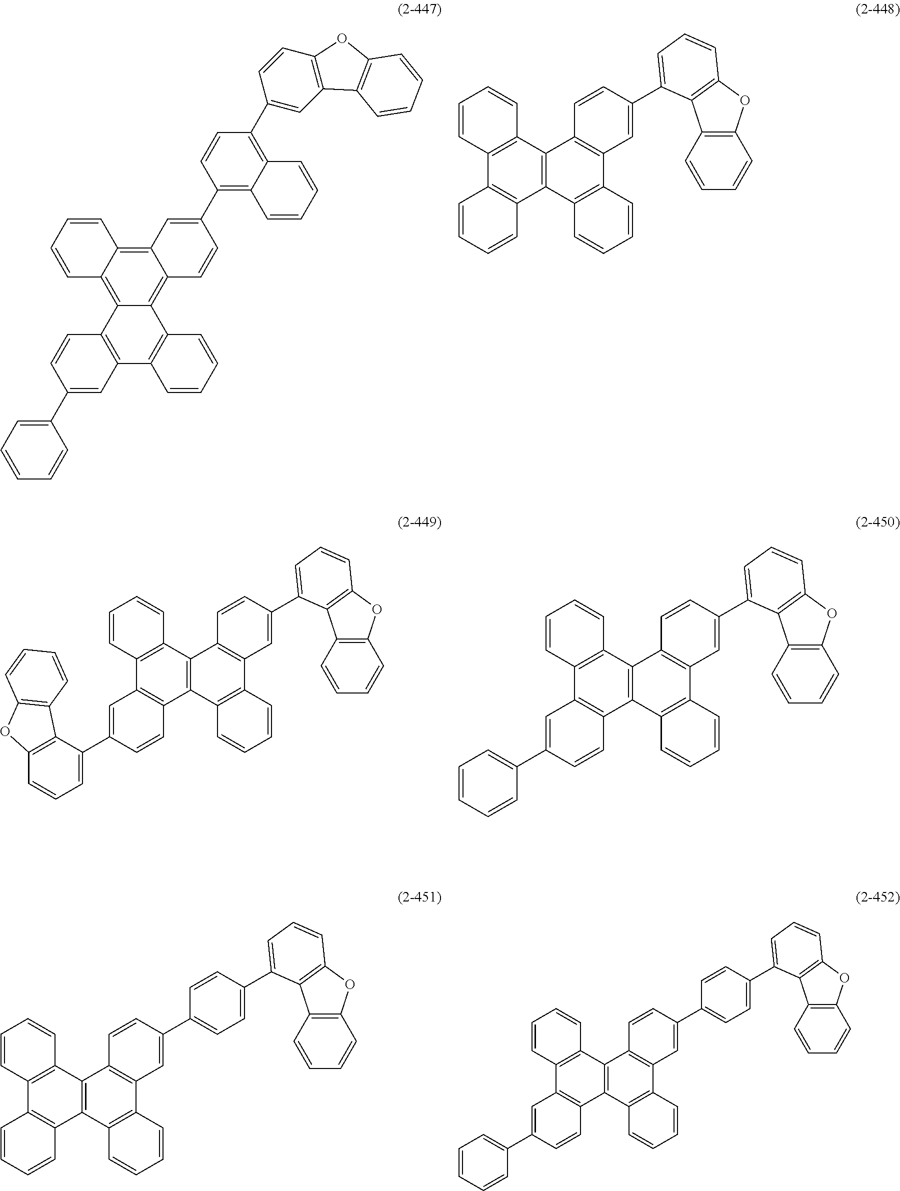

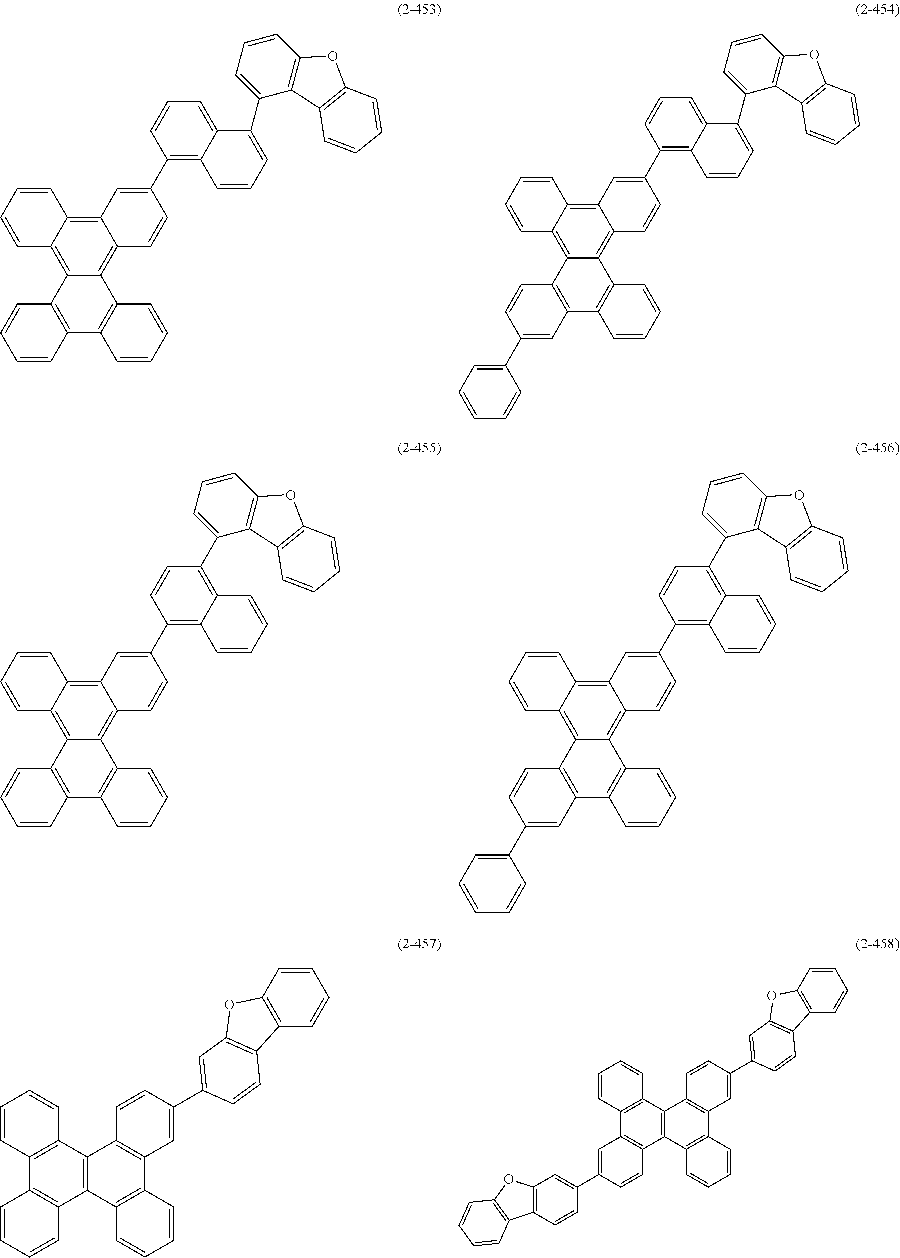

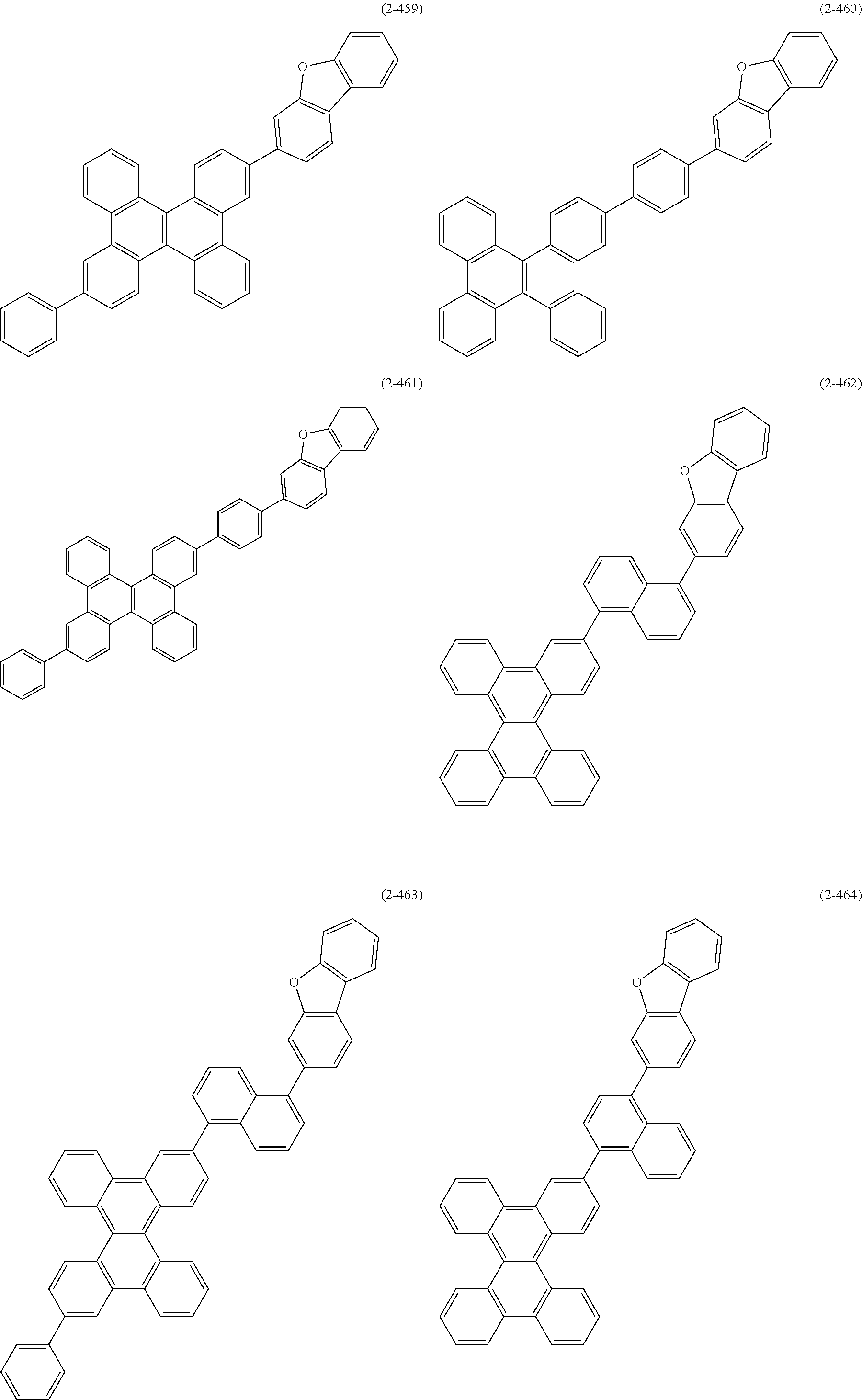

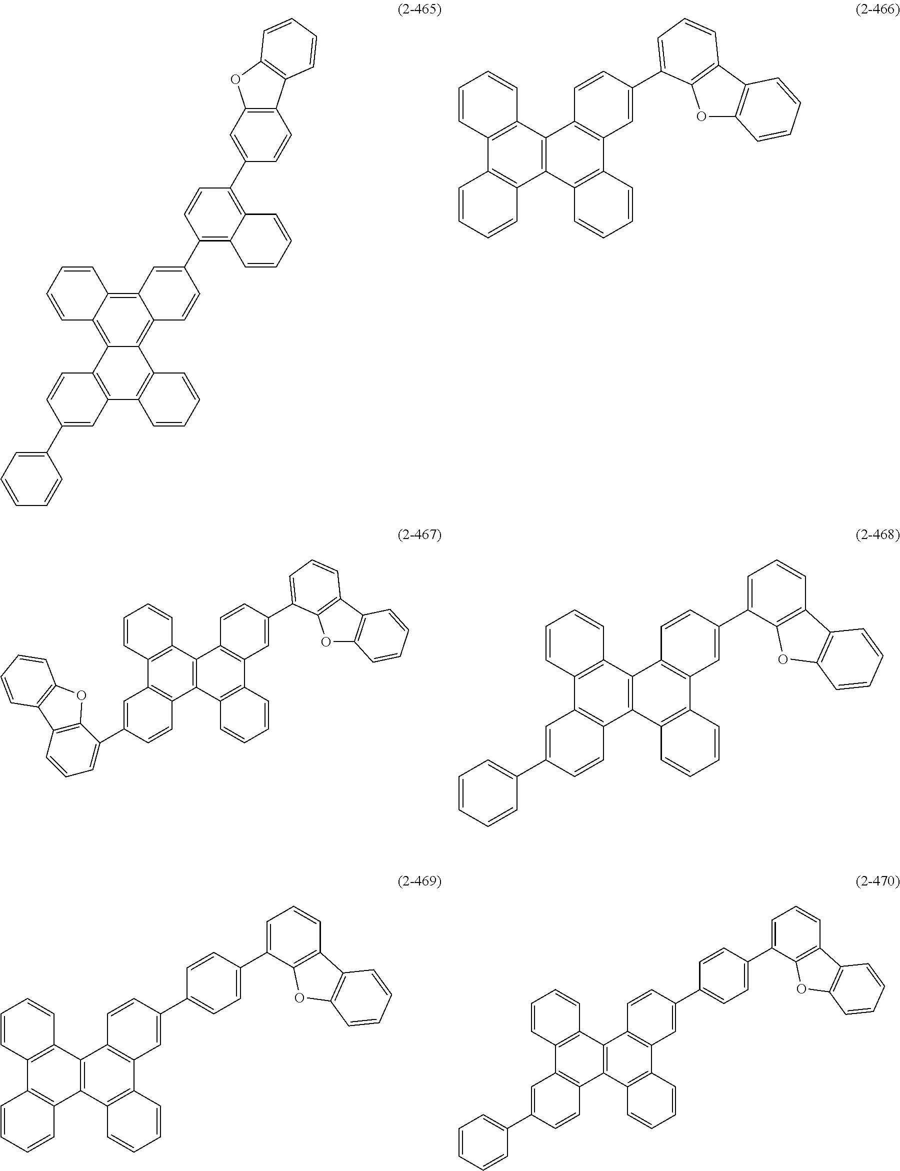 [SMILES: c1ccc(-c2ccc3c(c2)c2ccccc2c2c4ccc(-c5ccc(-c6ccc7c(c6)oc6ccccc67)c6ccccc56)cc4c4ccccc4c32)cc1 ; c1ccc2c(c1)oc1c(-c3ccc4c(c3)c3ccccc3c3c5ccccc5c5ccccc5c43)cccc12 ; c1ccc2c(c1)oc1c(-c3ccc4c(c3)c3ccccc3c3c5ccc(-c6cccc7c6oc6ccccc67)cc5c5ccccc5c43)cccc12 ; c1ccc(-c2ccc3c(c2)c2ccccc2c2c4ccc(-c5cccc6c5oc5ccccc56)cc4c4ccccc4c32)cc1 ; c1ccc2c(c1)oc1c(-c3ccc(-c4ccc5c(c4)c4ccccc4c4c6ccccc6c6ccccc6c54)cc3)cccc12 ; c1ccc(-c2ccc3c(c2)c2ccccc2c2c4ccc(-c5ccc(-c6cccc7c6oc6ccccc67)cc5)cc4c4ccccc4c32)cc1]

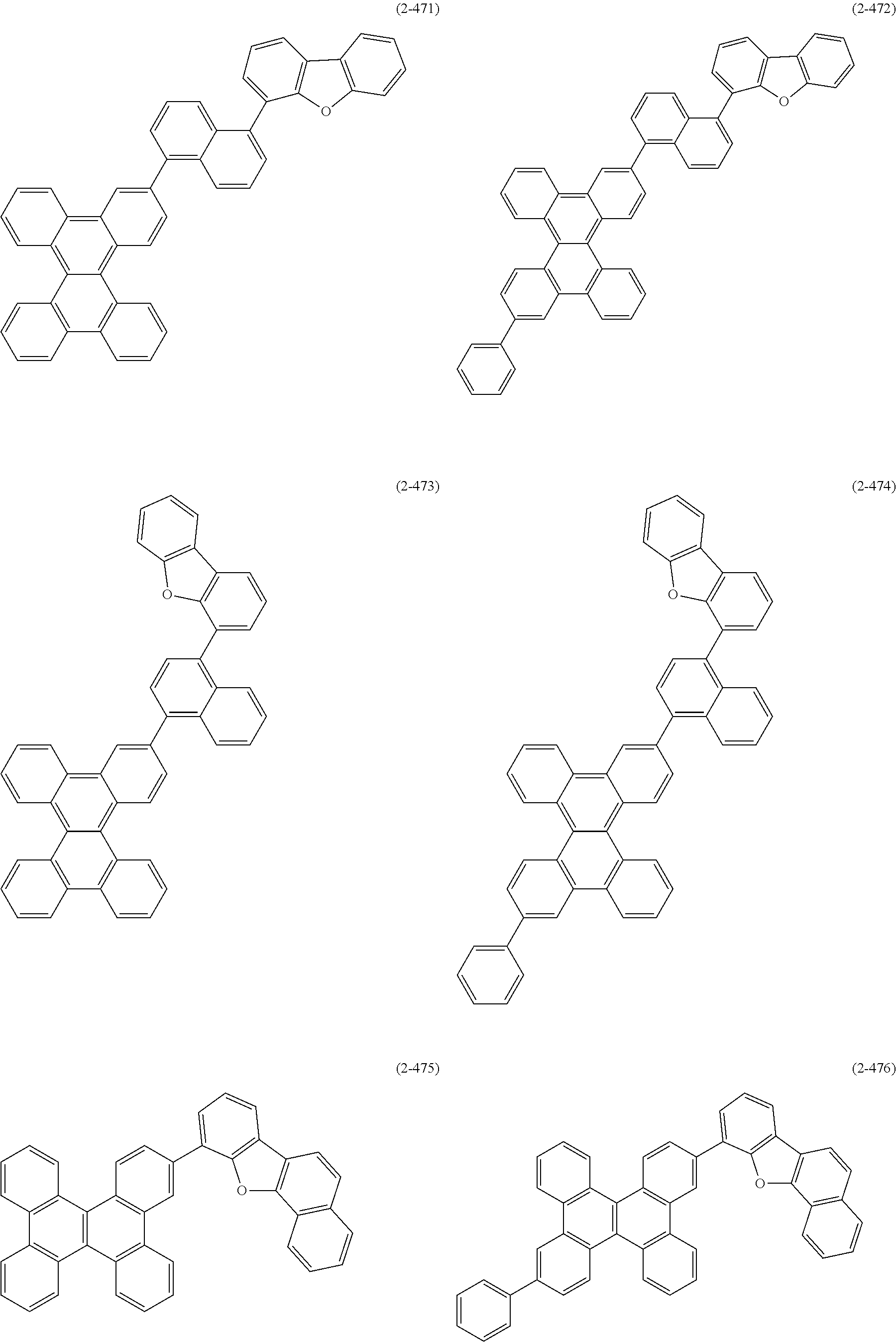

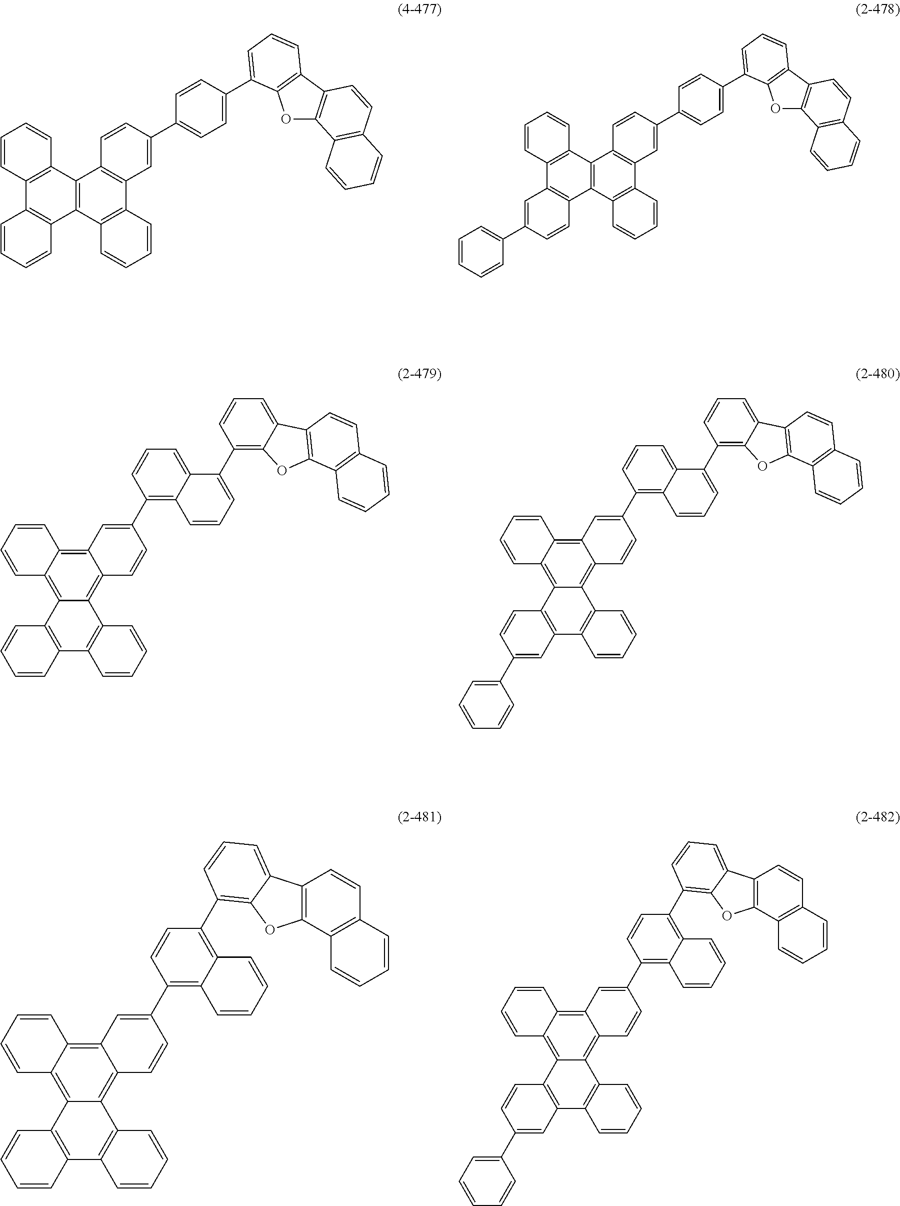

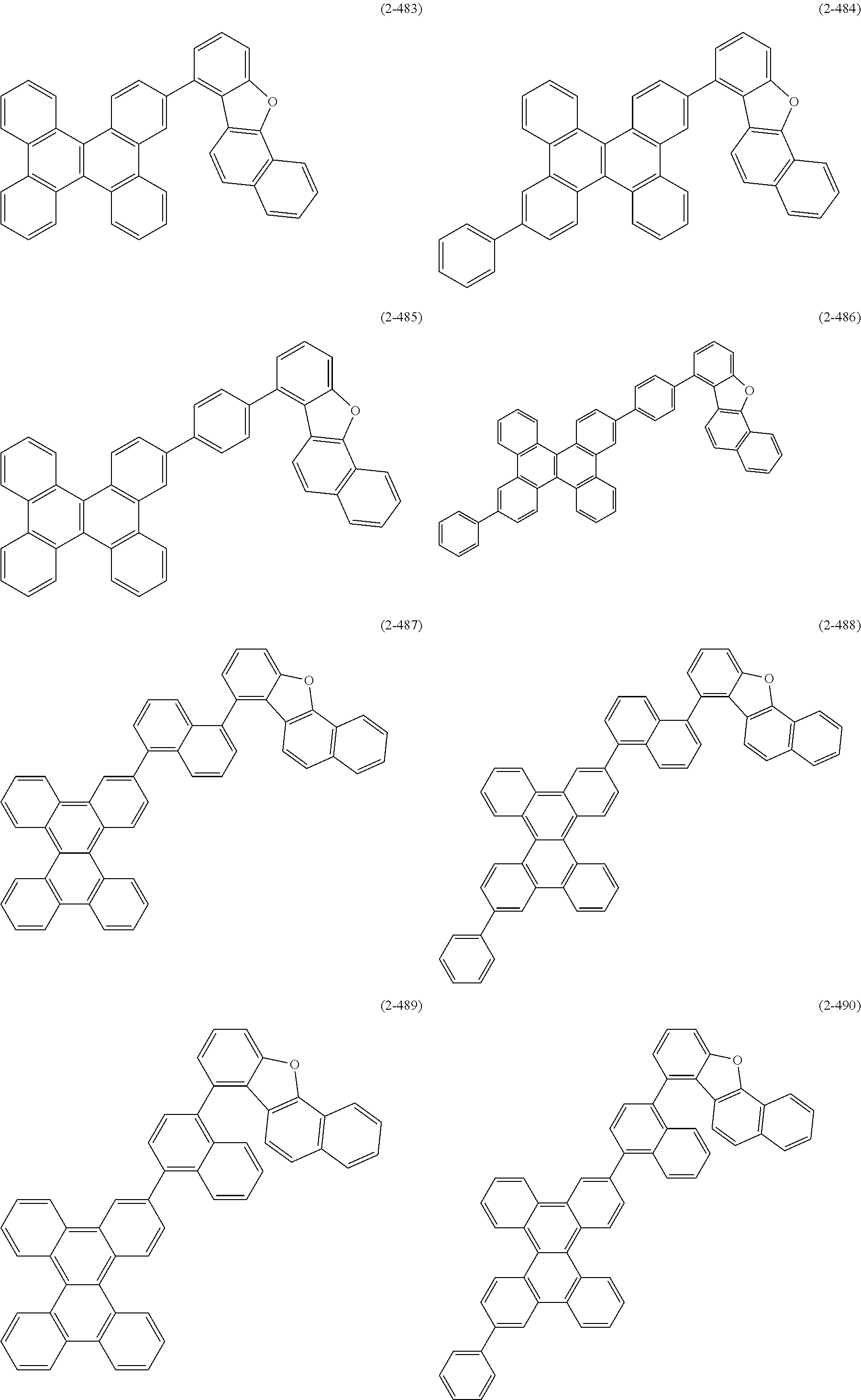

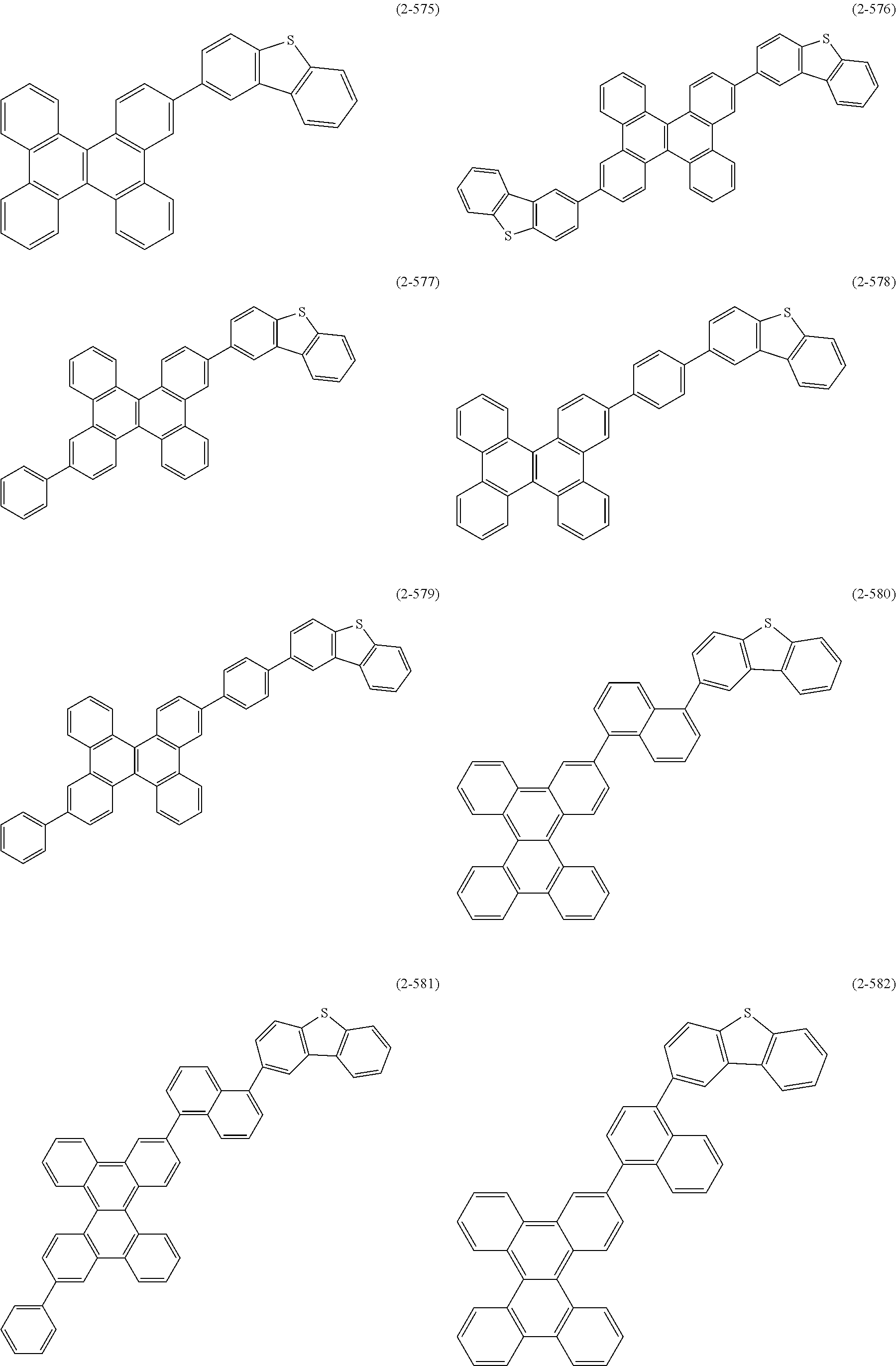

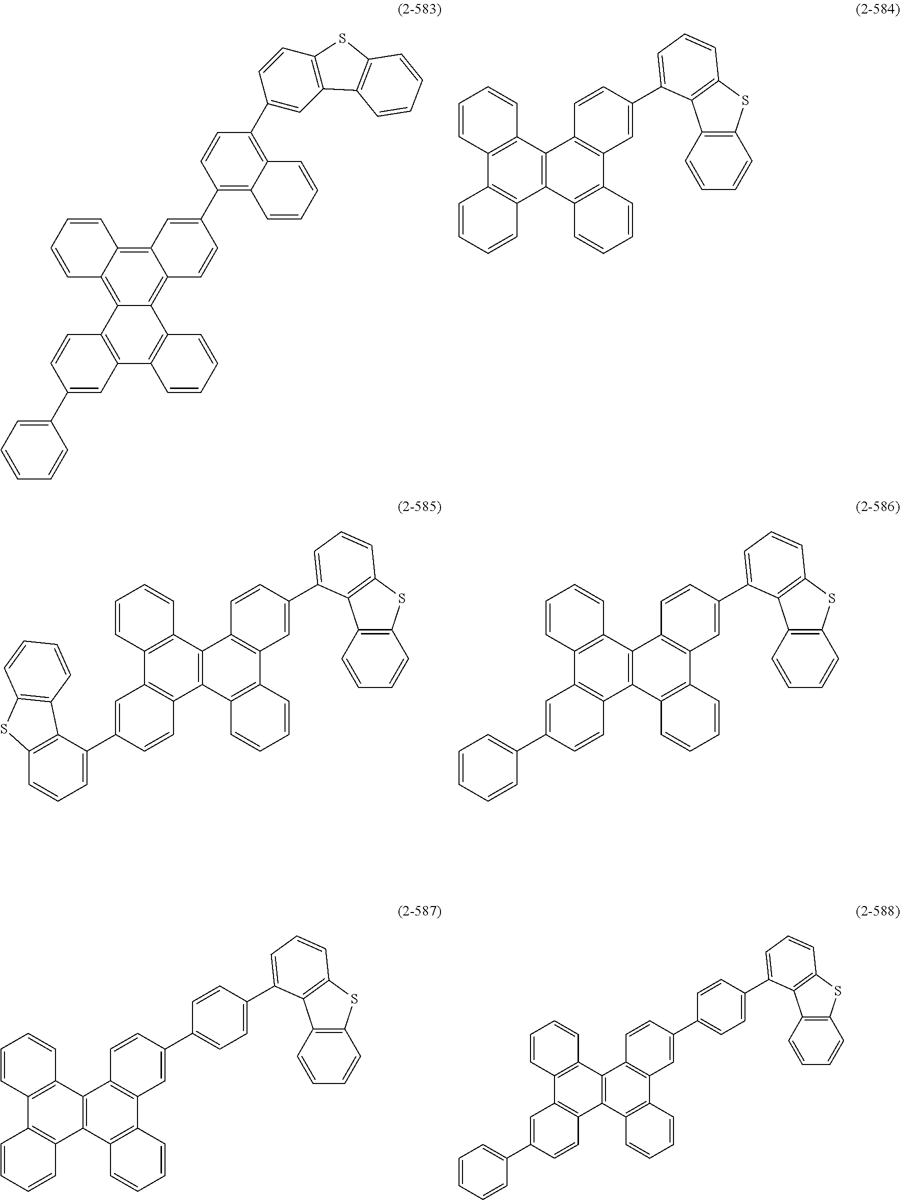

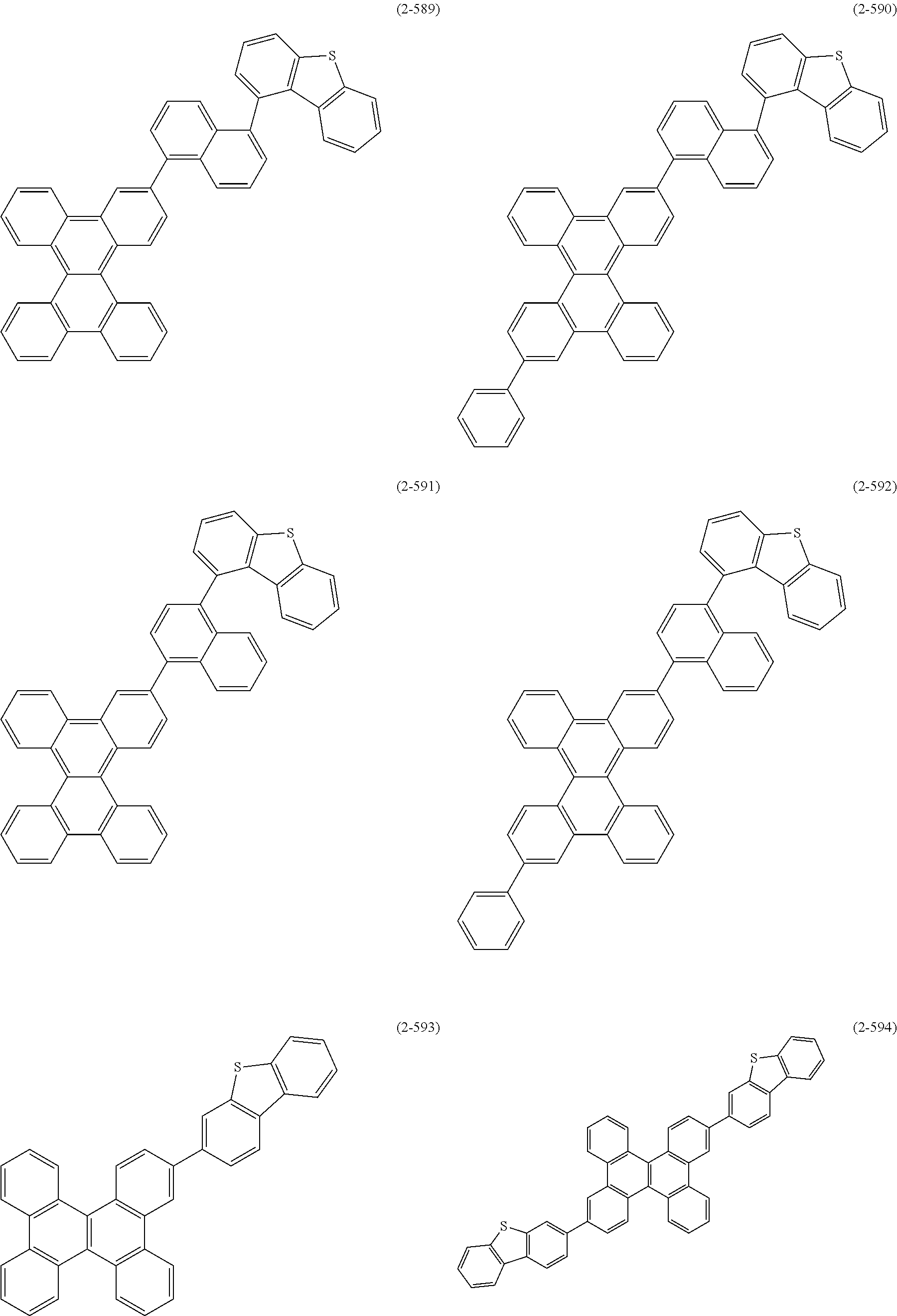

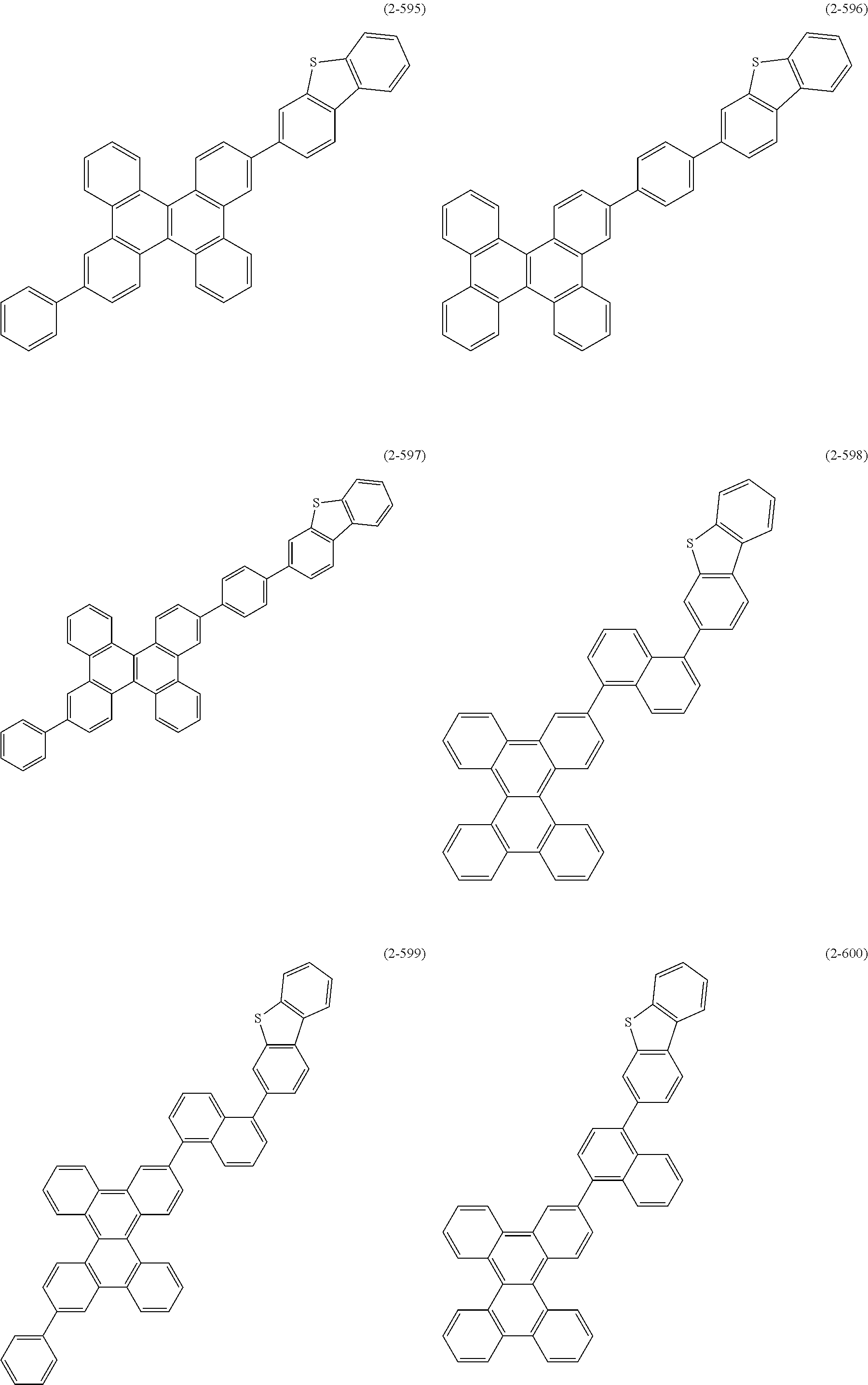

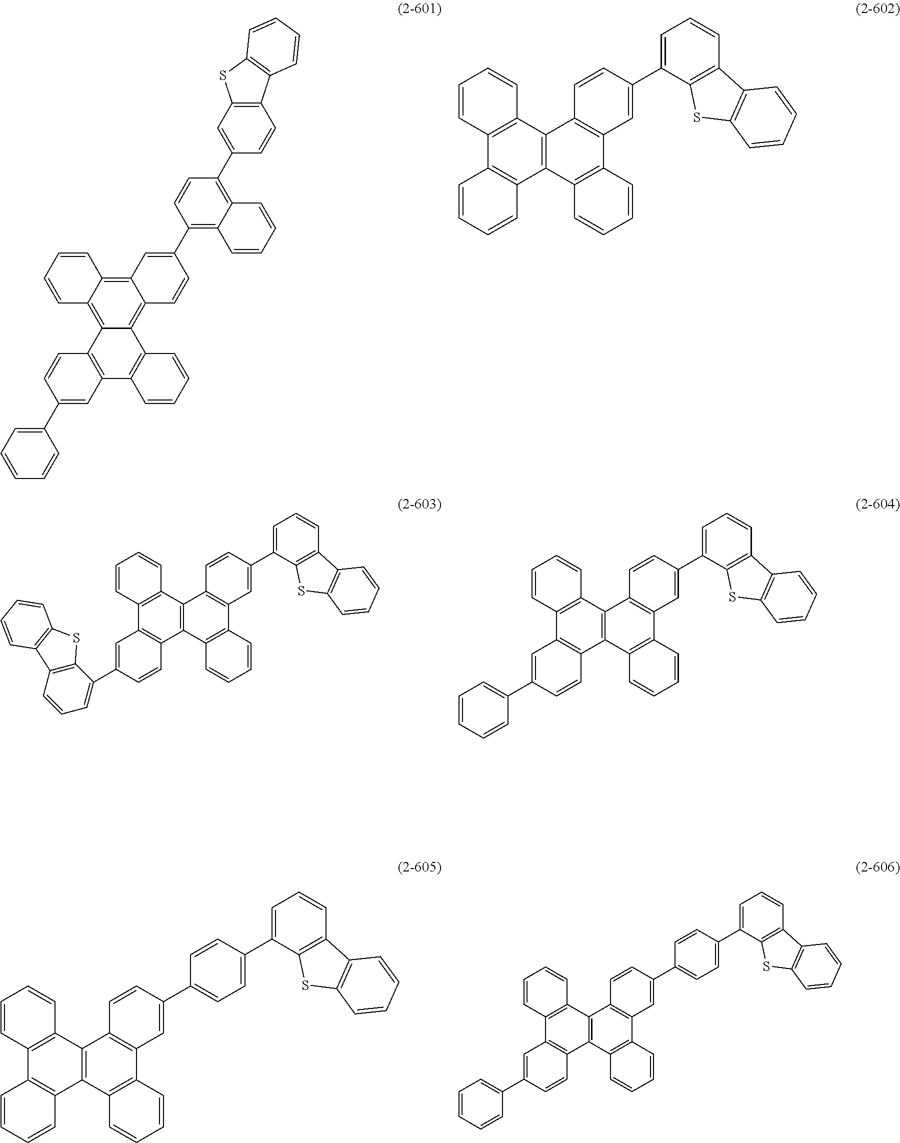 [SMILES: c1ccc(-c2ccc3c(c2)c2ccccc2c2c4ccc(-c5ccc(-c6ccc7c(c6)sc6ccccc67)c6ccccc56)cc4c4ccccc4c32)cc1 ; c1ccc2c(c1)sc1c(-c3ccc4c(c3)c3ccccc3c3c5ccccc5c5ccccc5c43)cccc12 ; c1ccc2c(c1)sc1c(-c3ccc4c(c3)c3ccccc3c3c5ccc(-c6cccc7c6sc6ccccc67)cc5c5ccccc5c43)cccc12 ; c1ccc(-c2ccc3c(c2)c2ccccc2c2c4ccc(-c5cccc6c5sc5ccccc56)cc4c4ccccc4c32)cc1 ; c1ccc2c(c1)sc1c(-c3ccc(-c4ccc5c(c4)c4ccccc4c4c6ccccc6c6ccccc6c54)cc3)cccc12 ; c1ccc(-c2ccc3c(c2)c2ccccc2c2c4ccc(-c5ccc(-c6cccc7c6sc6ccccc67)cc5)cc4c4ccccc4c32)cc1]

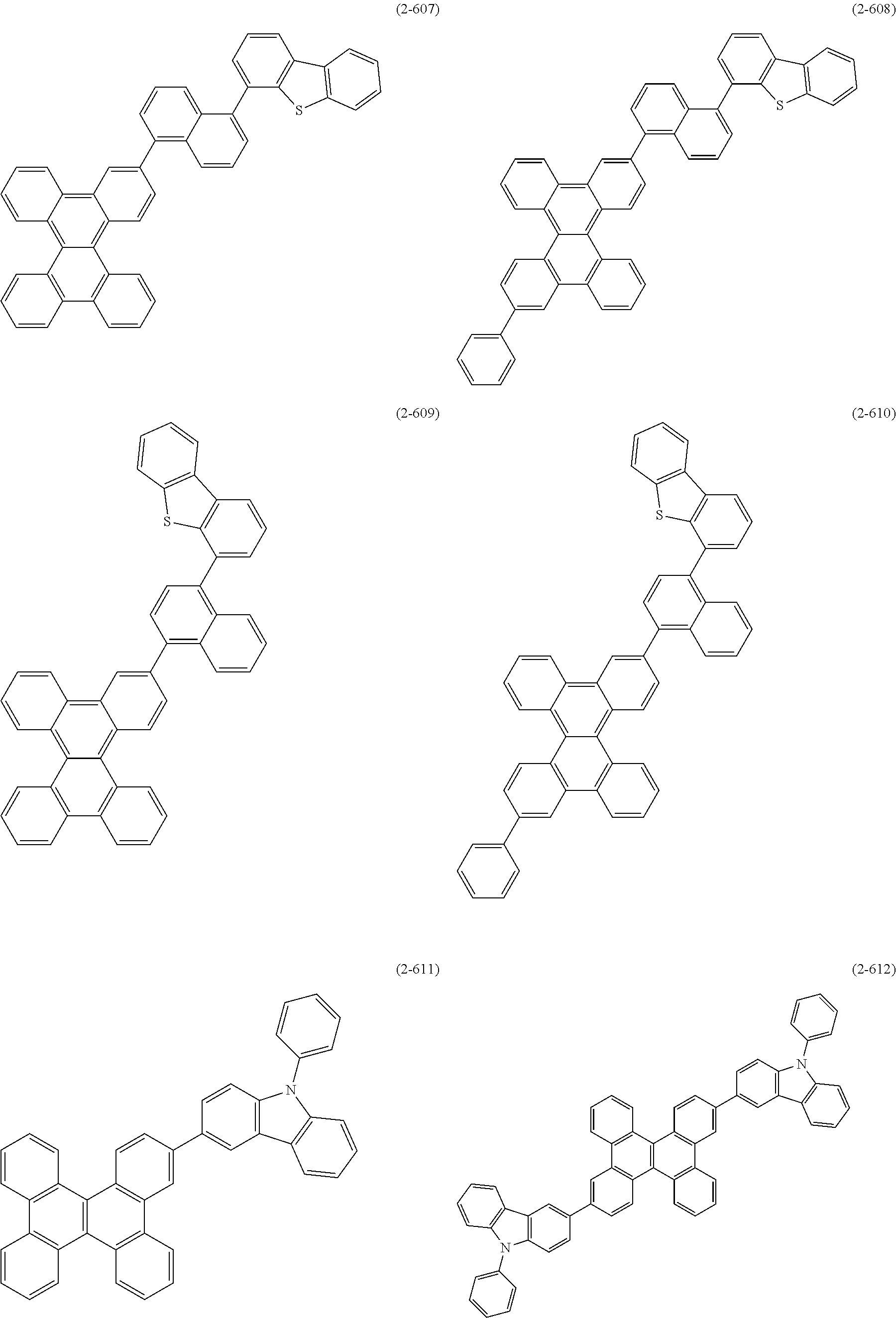

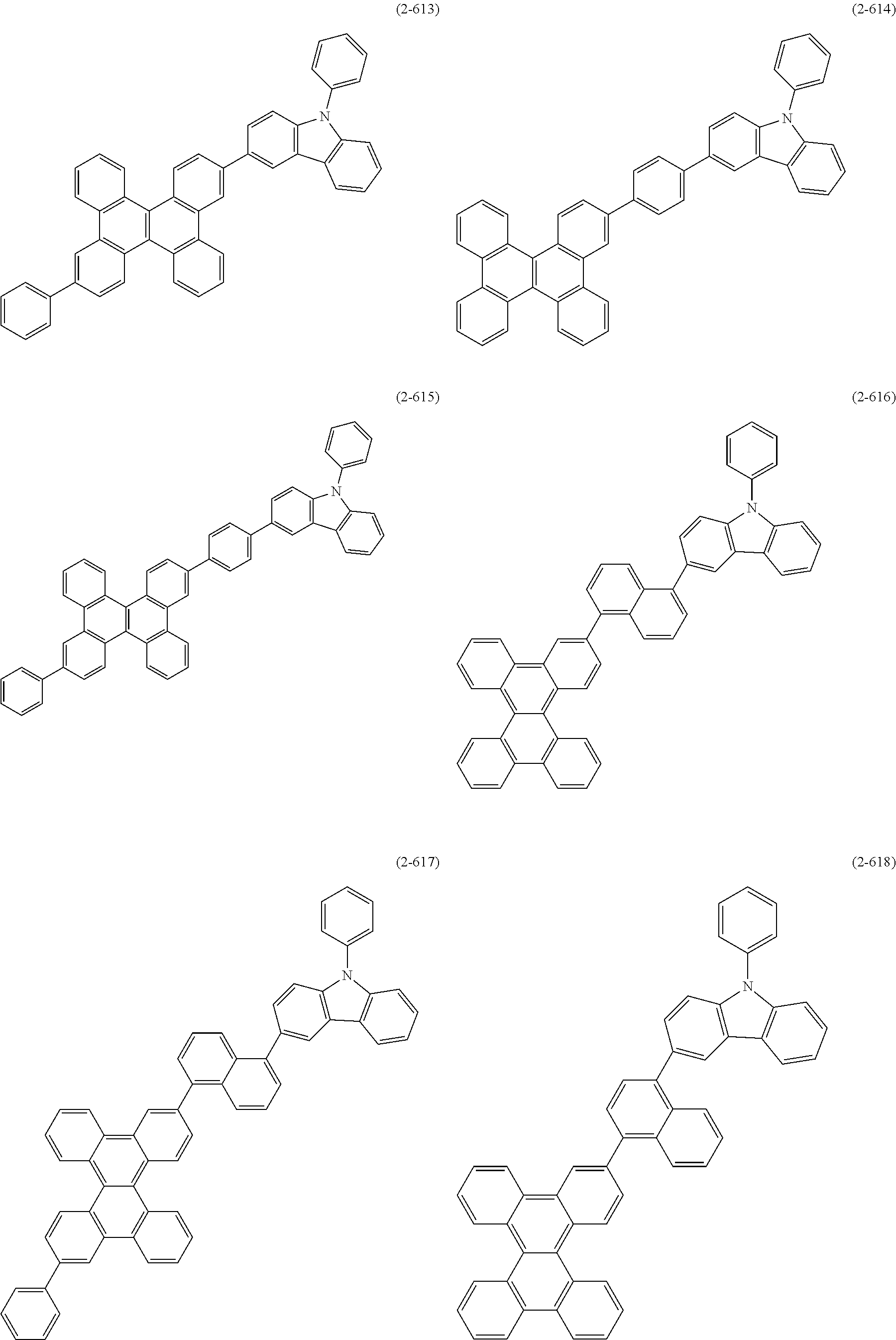

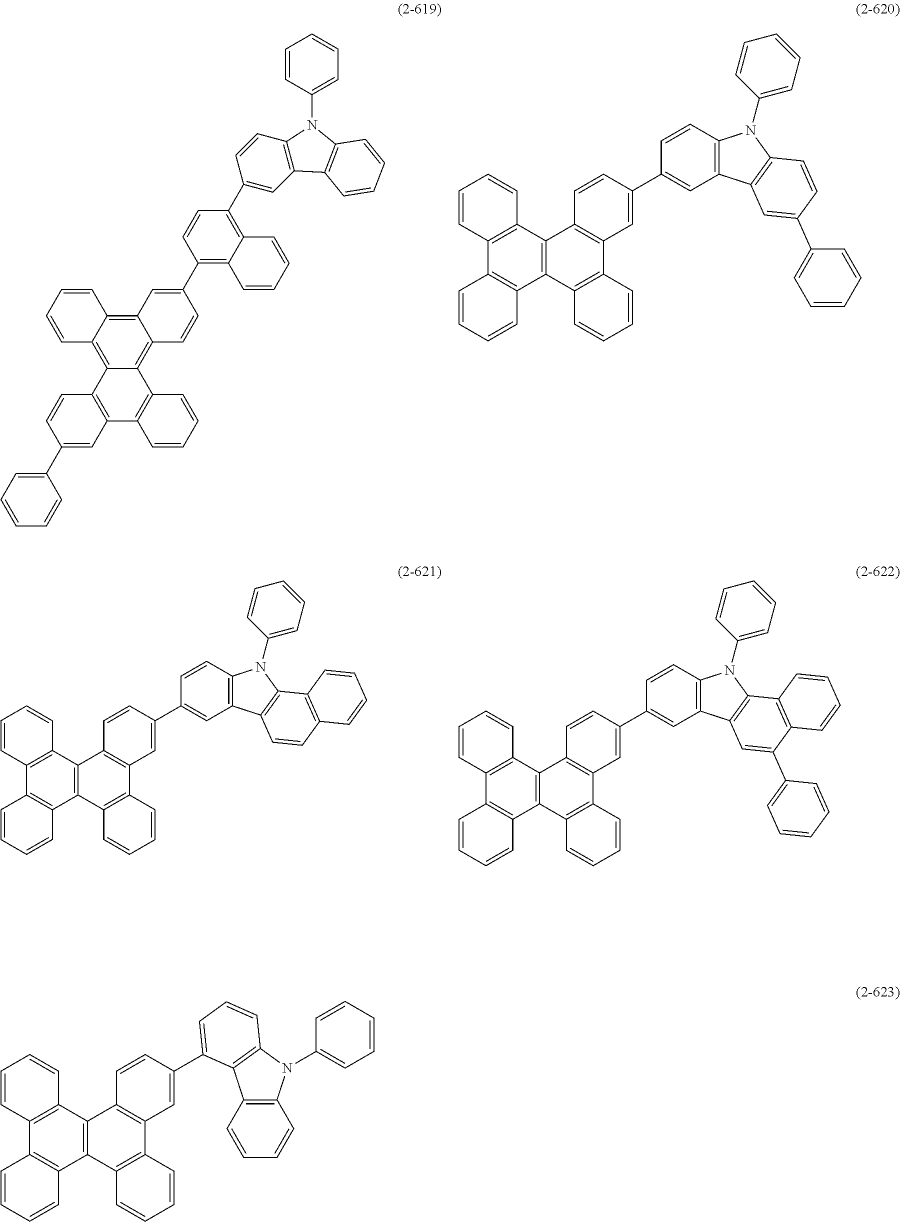 [SMILES: c1ccc(-c2ccc3c(c2)c2ccccc2c2c4ccc(-c5ccc(-c6ccc7c(c6)c6ccccc6n7-c6ccccc6)c6ccccc56)cc4c4ccccc4c32)cc1 ; c1ccc(-c2ccc3c(c2)c2cc(-c4ccc5c(c4)c4ccccc4c4c6ccccc6c6ccccc6c54)ccc2n3-c2ccccc2)cc1 ; c1ccc(-n2c3ccc(-c4ccc5c(c4)c4ccccc4c4c6ccccc6c6ccccc6c54)cc3c3ccc4ccccc4c32)cc1 ; c1ccc(-c2cc3c4cc(-c5ccc6c(c5)c5ccccc5c5c7ccccc7c7ccccc7c65)ccc4n(-c4ccccc4)c3c3ccccc23)cc1 ; c1ccc(-n2c3ccccc3c3c(-c4ccc5c(c4)c4ccccc4c4c6ccccc6c6ccccc6c54)cccc32)cc1]

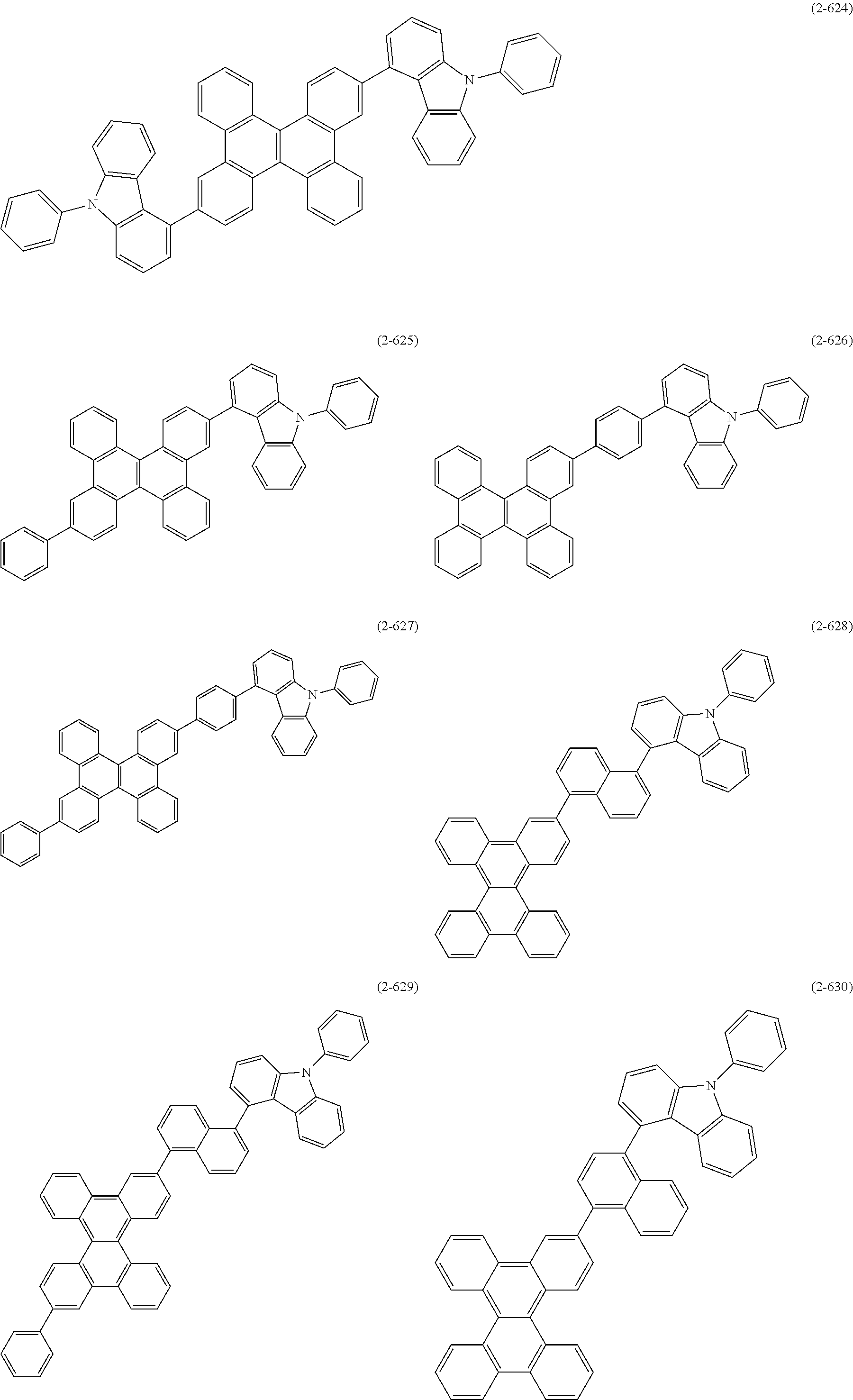

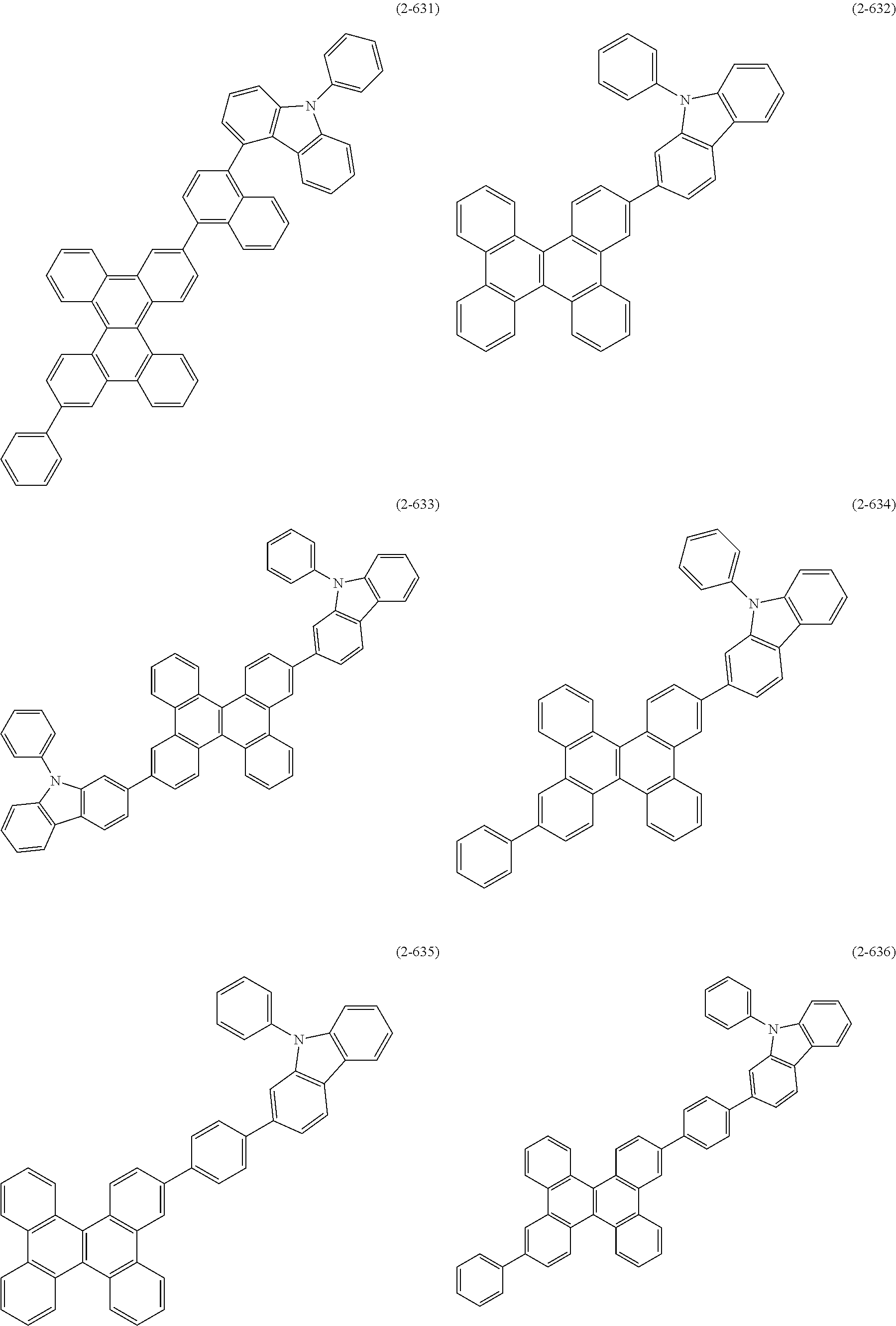

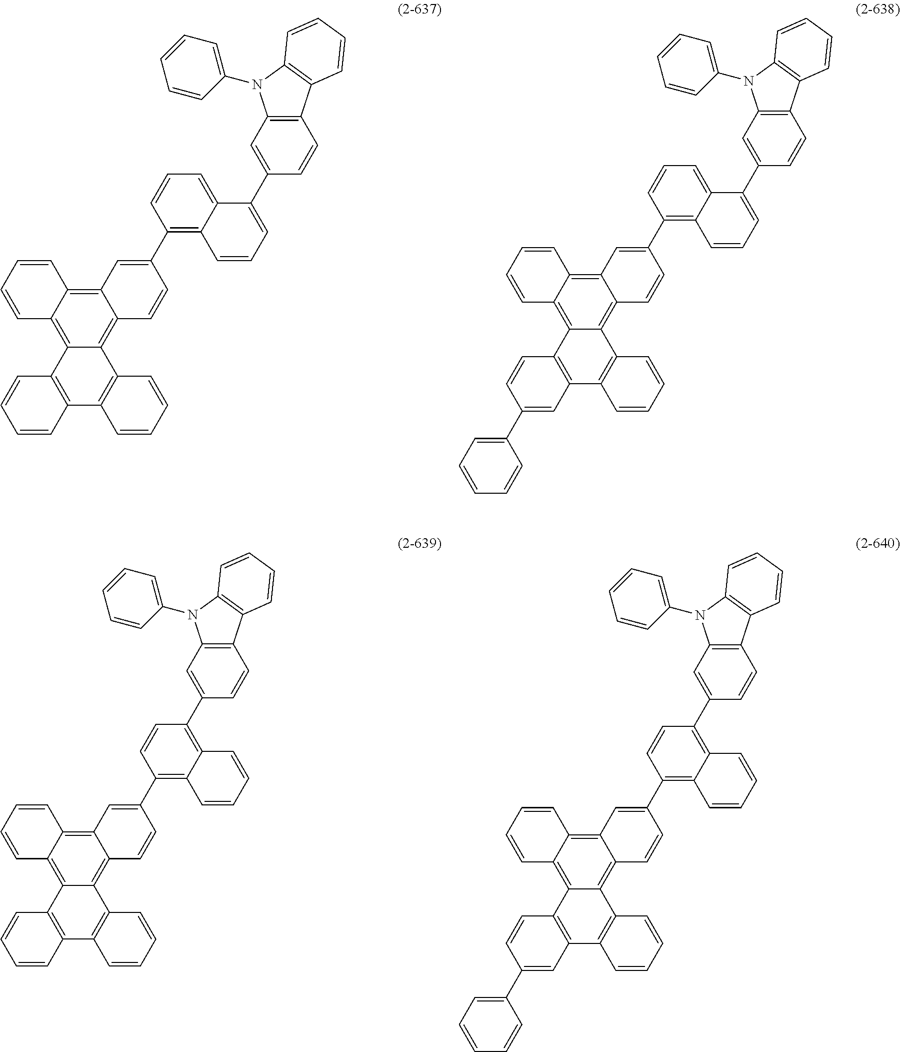 [SMILES: c1ccc(-n2c3ccccc3c3ccc(-c4cccc5c(-c6ccc7c(c6)c6ccccc6c6c8ccccc8c8ccccc8c76)cccc45)cc32)cc1 ; c1ccc(-c2ccc3c(c2)c2ccccc2c2c4ccc(-c5cccc6c(-c7ccc8c9ccccc9n(-c9ccccc9)c8c7)cccc56)cc4c4ccccc4c32)cc1 ; c1ccc(-n2c3ccccc3c3ccc(-c4ccc(-c5ccc6c(c5)c5ccccc5c5c7ccccc7c7ccccc7c65)c5ccccc45)cc32)cc1 ; c1ccc(-c2ccc3c(c2)c2ccccc2c2c4ccc(-c5ccc(-c6ccc7c8ccccc8n(-c8ccccc8)c7c6)c6ccccc56)cc4c4ccccc4c32)cc1]

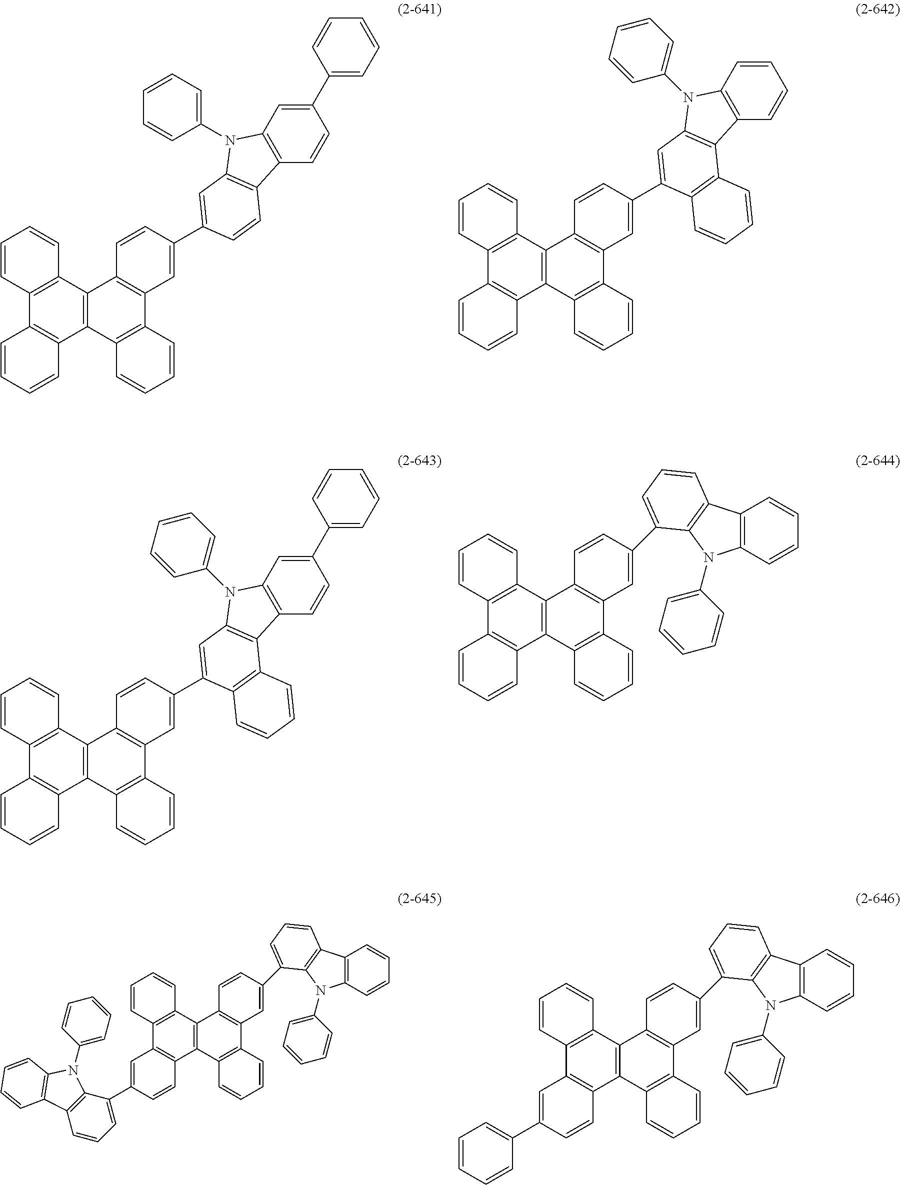

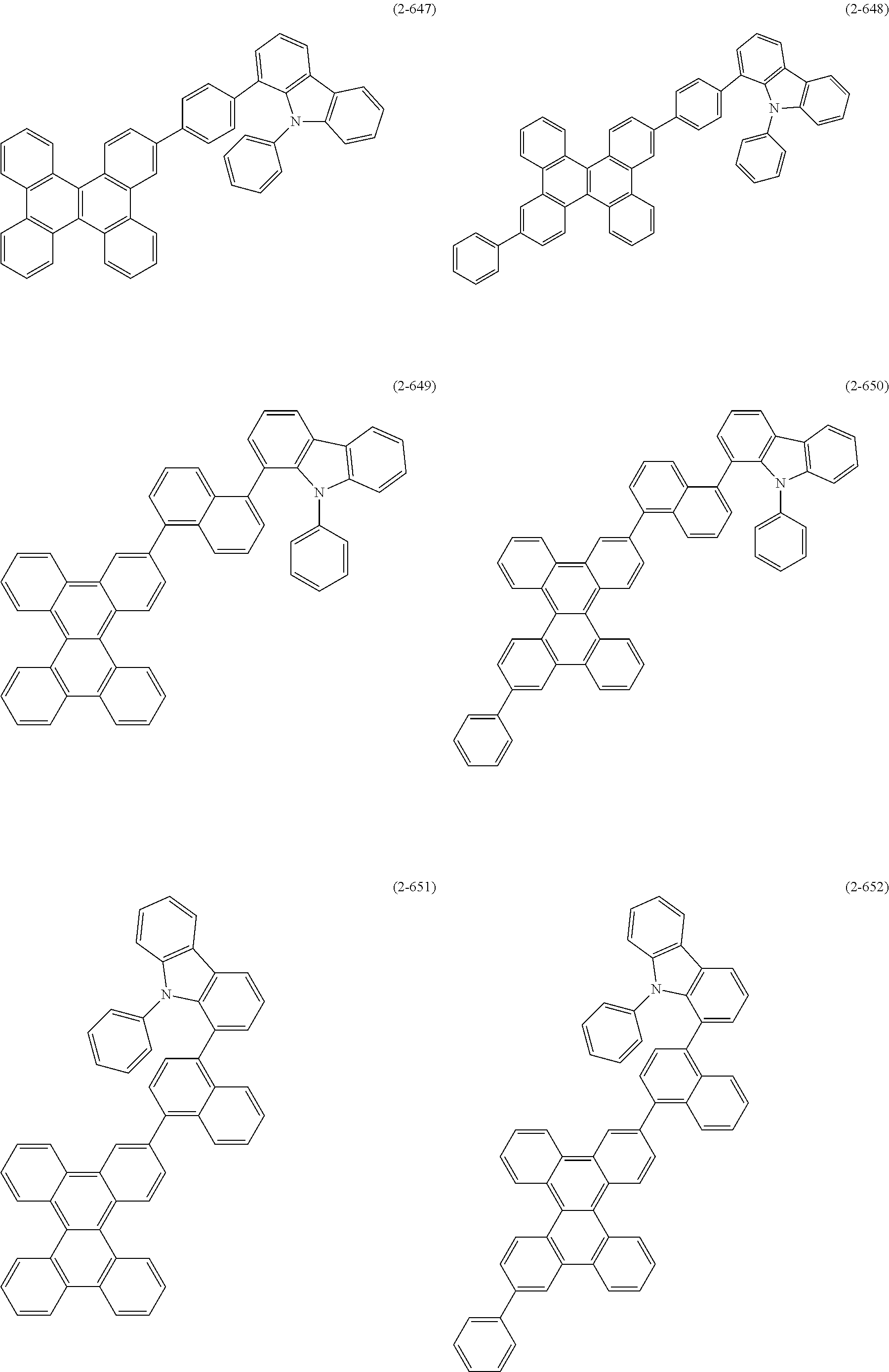

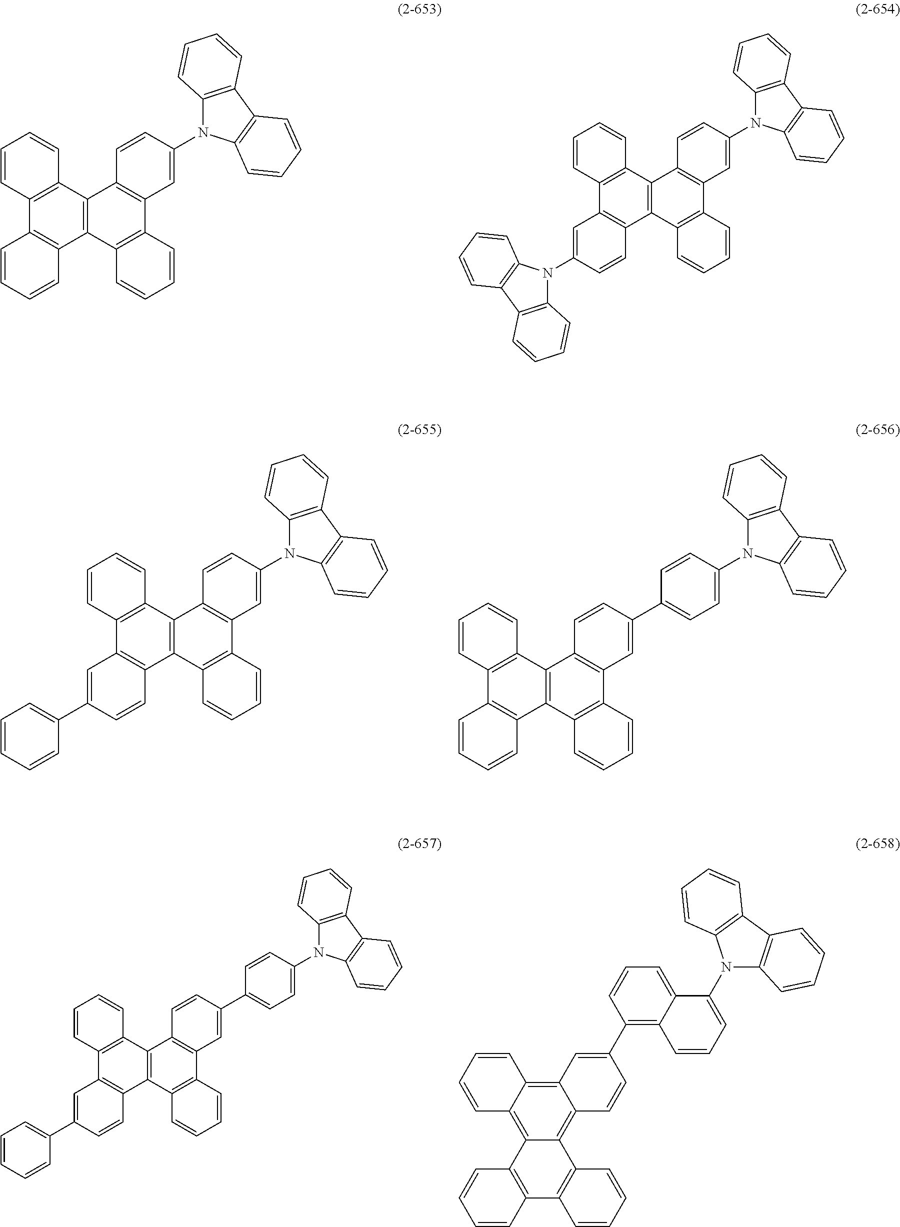 [SMILES: c1ccc2c(c1)c1ccccc1c1c3ccc(-n4c5ccccc5c5ccccc54)cc3c3ccccc3c21 ; c1ccc2c(c1)c1cc(-n3c4ccccc4c4ccccc43)ccc1c1c3ccccc3c3cc(-n4c5ccccc5c5ccccc54)ccc3c21 ; c1ccc(-c2ccc3c(c2)c2ccccc2c2c4ccc(-n5c6ccccc6c6ccccc65)cc4c4ccccc4c32)cc1 ; c1ccc2c(c1)c1ccccc1c1c3ccc(-c4ccc(-n5c6ccccc6c6ccccc65)cc4)cc3c3ccccc3c21 ; c1ccc(-c2ccc3c(c2)c2ccccc2c2c4ccc(-c5ccc(-n6c7ccccc7c7ccccc76)cc5)cc4c4ccccc4c32)cc1 ; c1cc(-n2c3ccccc3c3ccccc32)c2cccc(-c3ccc4c(c3)c3ccccc3c3c5ccccc5c5ccccc5c43)c2c1]

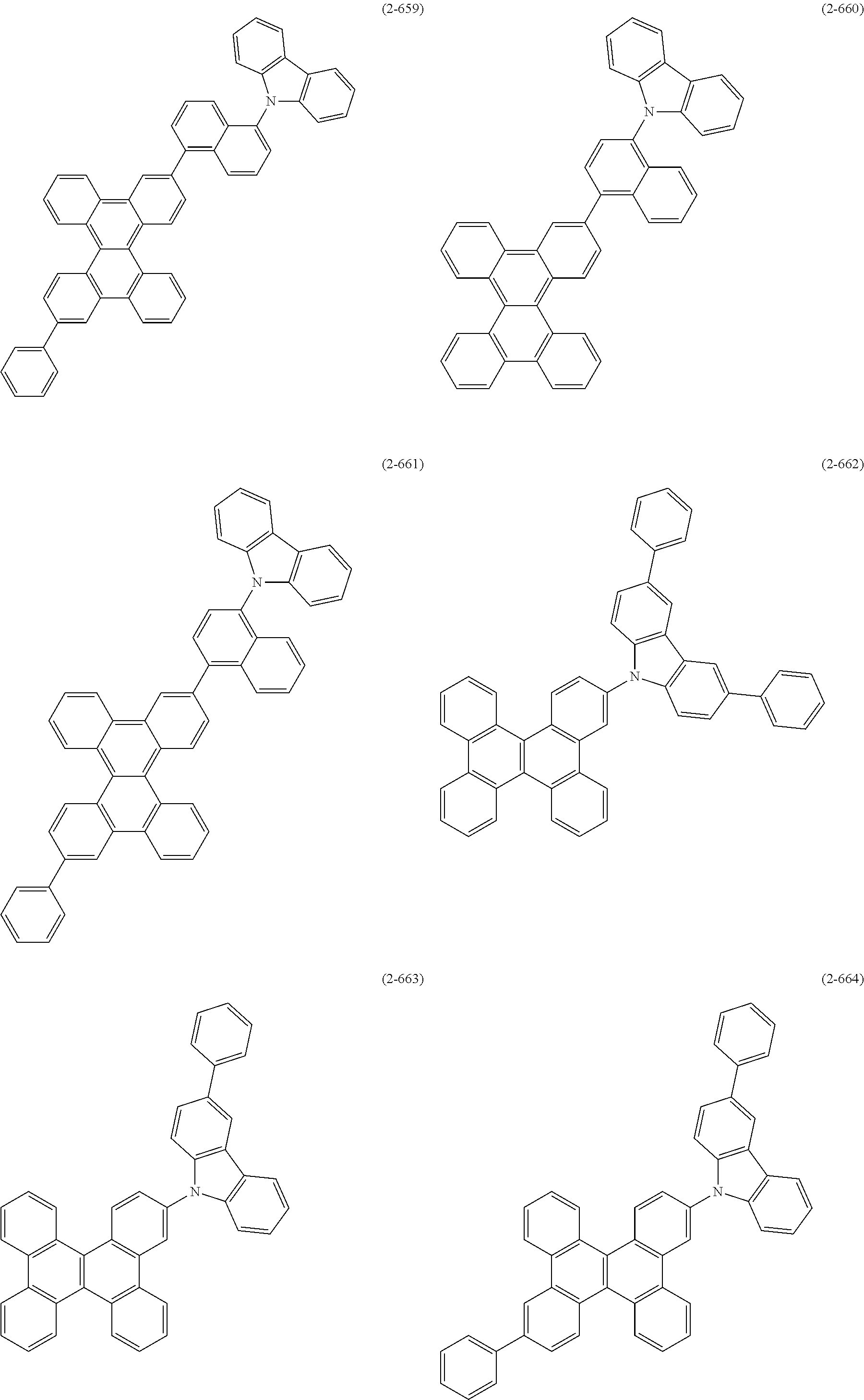

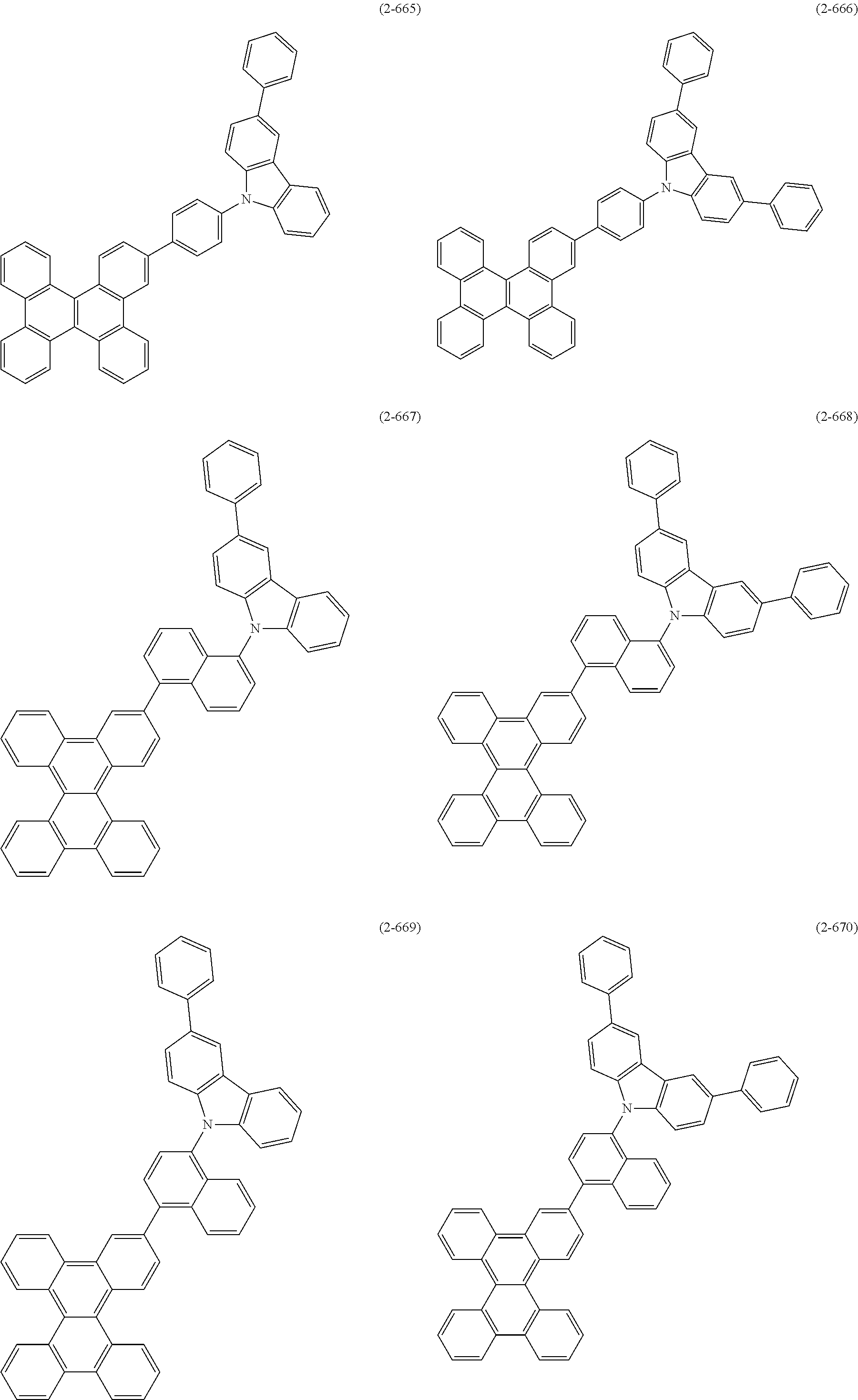

Among the above compounds, compounds represented by formulas (2-101) to (2-172), (2-201) to (2-203), (2-277) to (2-281), (2-301) to (2-372), (2-381) to (2-383), (2-401) to (2-490), (2-575), (2-577), (2-578), (2-580), (2-582), (2-584), (2-586), (2-587), (2-589), (2-591), (2-593), (2-595), (2-596), (2-598), (2-600), (2-602), (2-604), (2-605), (2-607), (2-609), (2-611), (2-613) to (2-623), (2-625) to (2-632), (2-634) to (2-644), (2-646) to (2-653), and (2-655) to (2-670) are preferable.

Compounds represented by formulas (2-101) to (2-103), (2-201) to (2-203), (2-301) to (2-303), (2-381) to (2-383), (2-401) to (2-490), and (2-611) to (2-670) are more preferable.

Compounds represented by formulas (2-301) to (2-303), (2-401), (2-411), (2-419), (2-427), (2-435), and (2-660) are particularly preferable.

Note that the present invention is not limited by the disclosure of the above specific structures.

1-4. Method for Manufacturing a Compound Represented by Formula (2)

The compound represented by formula (2) has a structure in which various substituents are bonded to a dibenzochrysene skeleton or the like, and can be manufactured by a known method. For example, the compound can be manufactured with reference to a manufacturing method (paragraphs [0066] to [0075]) and Synthesis Examples in Examples (paragraphs [0115] to [0131]) described in JP 2011-006397 A.

2. Organic Electroluminescent Element

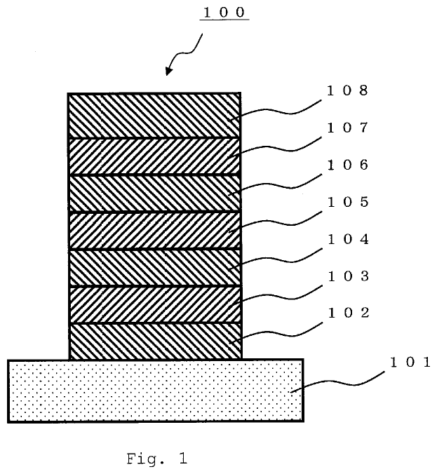

Hereinafter, an organic EL element according to the present embodiment will be described in detail based on the drawings. FIG. 1 is a schematic cross-sectional view illustrating the organic EL element according to the present embodiment.

<Structure of Organic Electroluminescent Element>

An organic EL element 100 illustrated in FIG. 1 includes a substrate 101, a positive electrode 102 provided on the substrate 101, a hole injection layer 103 provided on the positive electrode 102, a hole transport layer 104 provided on the hole injection layer 103, a light emitting layer 105 provided on the hole transport layer 104, an electron transport layer 106 provided on the light emitting layer 105, an electron injection layer 107 provided on the electron transport layer 106, and a negative electrode 108 provided on the electron injection layer 107.

Incidentally, the organic EL element 100 may be configured, by reversing the manufacturing order, to include, for example, the substrate 101, the negative electrode 108 provided on the substrate 101, the electron injection layer 107 provided on the negative electrode 108, the electron transport layer 106 provided on the electron injection layer 107, the light emitting layer 105 provided on the electron transport layer 106, the hole transport layer 104 provided on the light emitting layer 105, the hole injection layer 103 provided on the hole transport layer 104, and the positive electrode 102 provided on the hole injection layer 103.

Not all of the above layers are essential. The configuration includes the positive electrode 102, the light emitting layer 105, and the negative electrode 108 as a minimum constituent unit, and optionally includes the hole injection layer 103, the hole transport layer 104, the electron transport layer 106, and the electron injection layer 107. Each of the above layers may be formed of a single layer or a plurality of layers.

A form of layers constituting the organic EL element may be, in addition to the above structure form of “substrate/positive electrode/hole injection layer/hole transport layer/light emitting layer/electron transport layer/electron injection layer/negative electrode”, a structure form of “substrate/positive electrode/hole transport layer/light emitting layer/electron transport layer/electron injection layer/negative electrode”, “substrate/positive electrode/hole injection layer/light emitting layer/electron transport layer/electron injection layer/negative electrode”, “substrate/positive electrode/hole injection layer/hole transport layer/light emitting layer/electron injection layer/negative electrode”, “substrate/positive electrode/hole injection layer/hole transport layer/light emitting layer/electron transport layer/negative electrode”, “substrate/positive electrode/light emitting layer/electron transport layer/electron injection layer/negative electrode”, “substrate/positive electrode/hole transport layer/light emitting layer/electron injection layer/negative electrode”, “substrate/positive electrode/hole transport layer/light emitting layer/electron transport layer/negative electrode”, “substrate/positive electrode/hole injection layer/light emitting layer/electron injection layer/negative electrode”, “substrate/positive electrode/hole injection layer/light emitting layer/electron transport layer/negative electrode”, “substrate/positive electrode/light emitting layer/electron transport layer/negative electrode”, or “substrate/positive electrode/light emitting layer/electron injection layer/negative electrode”.

<Substrate in Organic Electroluminescent Element>

The substrate 101 serves as a support of the organic EL element 100, and usually, quartz, glass, metals, plastics, and the like are used therefor. The substrate 101 is formed into a plate shape, a film shape, or a sheet shape according to a purpose, and for example, a glass plate, a metal plate, a metal foil, a plastic film, and a plastic sheet are used therefor. Among these examples, a glass plate and a plate made of a transparent synthetic resin such as polyester, polymethacrylate, polycarbonate, or polysulfone are preferable. For a glass substrate, soda lime glass, alkali-free glass, and the like are used. The thickness is only required to be sufficient for maintaining mechanical strength. Therefore, the thickness is only required to be 0.2 mm or more, for example. An upper limit value of the thickness is, for example, 2 mm or less, and preferably 1 mm or less. Regarding a material of glass, glass having fewer ions eluted from the glass is desirable, and therefore alkali-free glass is preferable. However, soda lime glass which has been subjected to barrier coating with SiO₂ or the like is also commercially available, and therefore this soda lime glass can be used. Furthermore, the substrate 101 may be provided with a gas barrier film such as a dense silicon oxide film on at least one surface in order to increase a gas barrier property. Particularly in a case of using a plate, a film, or a sheet made of a synthetic resin having a low gas barrier property as the substrate 101, a gas barrier film is preferably provided.

<Positive Electrode in Organic Electroluminescent Element>

The positive electrode 102 plays a role of injecting a hole into the light emitting layer 105. Incidentally, in a case where the hole injection layer 103 and/or the hole transport layer 104 are/is disposed between the positive electrode 102 and the light emitting layer 105, a hole is injected into the light emitting layer 105 through these layers.

Examples of a material to form the positive electrode 102 include an inorganic compound and an organic compound. Examples of the inorganic compound include a metal (aluminum, gold, silver, nickel, palladium, chromium, and the like), a metal oxide (indium oxide, tin oxide, indium-tin oxide (ITO), indium-zinc oxide (IZO), and the like), a metal halide (copper iodide and the like), copper sulfide, carbon black, ITO glass, and Nesa glass. Examples of the organic compound include an electrically conductive polymer such as polythiophene such as poly(3-methylthiophene), polypyrrole, or polyaniline. In addition to these compounds, a material can be appropriately selected for use from materials used as a positive electrode of an organic EL element.

A resistance of a transparent electrode is not limited as long as a sufficient current can be supplied for light emission of a luminescent element. However, a low resistance is desirable from a viewpoint of consumption power of the luminescent element. For example, an ITO substrate having a resistance of 300Ω/□ or less functions as an element electrode. However, a substrate having a resistance of about 10Ω/□ can be also supplied at present, and therefore it is particularly desirable to use a low resistance product having a resistance of, for example, 100 to 5Ω/□, preferably 50 to 5Ω/□. The thickness of ITO can be arbitrarily selected according to a resistance value, but an ITO having a thickness of 50 to 300 nm is often used.

<Hole Injection Layer and Hole Transport Layer in Organic Electroluminescent Element>

The hole injection layer 103 plays a role of efficiently injecting a hole that migrates from the positive electrode 102 into the light emitting layer 105 or the hole transport layer 104. The hole transport layer 104 plays a role of efficiently transporting a hole injected from the positive electrode 102 or a hole injected from the positive electrode 102 through the hole injection layer 103 to the light emitting layer 105. The hole injection layer 103 and the hole transport layer 104 are each formed by laminating and mixing one or more kinds of hole injection/transport materials, or by a mixture of a hole injection/transport material and a polymer binder. Furthermore, a layer may be formed by adding an inorganic salt such as iron(III) chloride to the hole injection/transport material.

A hole injection/transport substance needs to efficiently inject/transport a hole coming from a positive electrode between electrodes to which an electric field is applied, and desirably has a high hole injection efficiency and transports an injected hole efficiently. For this purpose, a substance which has low ionization potential, large hole mobility, and further has excellent stability, and in which impurities serving as traps are not easily generated at the time of manufacturing and at the time of use, is preferable.

As a material to form the hole injection layer 103 and the hole transport layer 104, any compound can be selected for use among compounds that have been conventionally used as charge transporting materials for holes, p-type semiconductors, and known compounds used in a hole injection layer and a hole transport layer of an organic EL element. Specific examples thereof include a heterocyclic compound including a carbazole derivative (N-phenylcarbazole, polyvinylcarbazole, and the like), a biscarbazole derivative such as bis(N-arylcarbazole) or bis(N-alkylcarbazole), a triarylamine derivative (a polymer having an aromatic tertiary amino in a main chain or a side chain, 1,1-bis(4-di-p-tolylaminophenyl) cyclohexane, N,N′-diphenyl-N,N′-di(3-methylphenyl)-4,4′-diaminobiphenyl, N,N′-diphenyl-N,N′-dinaphthyl-4,4′-diaminobiphenyl, N,N′-diphenyl-N,N′-di(3-methylphenyl)-4,4′-diphenyl-1,1′-diamine, N,N′-dinaphthyl-N,N′-diphenyl-4,4′-dphenyl-1,1′-diamine, N⁴,N⁴′-diphenyl-N⁴,N⁴′-bis(9-phenyl-9H-carbazol-3-yl)-[1,1′-biphenyl]-4,4′-diamine, N⁴,N⁴,N⁴′,N⁴′-tetra[1,1′-biphenyl]-4-yl)-[1,1′-biphenyl]-4,4′-diamine, a triphenylamine derivative such as 4,4′,4″-tris(3-methylphenyl(phenyl)amino) triphenylamine, a starburst amine derivative, and the like), a stilbene derivative, a phthalocyanine derivative (non-metal, copper phthalocyanine, and the like), a pyrazoline derivative, a hydrazone-based compound, a benzofuran derivative, a thiophene derivative, an oxadiazole derivative, a quinoxaline derivative (for example, 1,4,5,8,9,12-hexaazatriphenylene-2,3,6,7,10,11-hexacarbonitrile, and the like), and a porphyrin derivative, and a polysilane. Among the polymer-based materials, a polycarbonate, a styrene derivative, a polyvinylcarbazole, a polysilane, and the like having the above monomers in side chains are preferable. However, there is no particular limitation as long as a compound can form a thin film required for manufacturing a luminescent element, can inject a hole from a positive electrode, and can further transport a hole.

Furthermore, it is also known that electroconductivity of an organic semiconductor is strongly affected by doping into the organic semiconductor. Such an organic semiconductor matrix substance is formed of a compound having a good electron-donating property, or a compound having a good electron-accepting property. For doping with an electron-donating substance, a strong electron acceptor such as tetracyanoquinonedimethane (TCNQ) or 2,3,5,6-tetrafluorotetracyano-1,4-benzoquinonedimethane (F4TCNQ) is known (see, for example, literature “M. Pfeiffer, A. Beyer, T. Fritz, K. Leo, Appl. Phys. Lett., 73(22), 3202-3204 (1998)” and literature “J. Blochwitz, M. Pheiffer, T. Fritz, K. Leo, Appl. Phys. Lett., 73(6), 729-731 (1998)”). These compounds generate a so-called hole by an electron migrating process in an electron-donating type base substance (hole transport substance). Electroconductivity of the base substance depends on the number and mobility of the holes fairly significantly. Known examples of a matrix substance having a hole transporting characteristic include a benzidine derivative (TPD and the like), a starburst amine derivative (TDATA and the like), and a specific metal phthalocyanine (particularly, zinc phthalocyanine (ZnPc) and the like) (JP 2005-167175 A).

<Light Emitting Layer in Organic Electroluminescent Element>

The light emitting layer 105 emits light by recombining a hole injected from the positive electrode 102 and an electron injected from the negative electrode 108 between electrodes to which an electric field is applied. A material to form the light emitting layer 105 is only required to be a compound which is excited by recombination between a hole and an electron and emits light (luminescent compound), and is preferably a compound which can form a stable thin film shape and exhibits a strong light emission (fluorescence) efficiency in a solid state. In the present invention, as a material for a light emitting layer, a compound represented by the above general formula (1) and a multimer thereof as a dopant material and a compound represented by the above general formula (2) as a host material can be used.

The light emitting layer may be formed of a single layer or a plurality of layers, and each layer is formed of a material for a light emitting layer (a host material and a dopant material). Each of the host material and the dopant material may be formed of a single kind, or a combination of a plurality of kinds. The dopant material may be included in the host material wholly or partially. Regarding a doping method, doping can be performed by a co-deposition method with a host material, or alternatively, a dopant material may be mixed in advance with a host material, and then vapor deposition may be performed simultaneously.

The amount of use of a host material depends on the kind of the host material, and is only required to be determined according to a characteristic of the host material. The reference of the amount of use of a host material is preferably from 50 to 99.999% by weight, more preferably from 80 to 99.95% by weight, and still more preferably from 90 to 99.9% by weight with respect to the total amount of a material for a light emitting layer.

The amount of use of a dopant material depends on the kind of the dopant material, and is only required to be determined according to a characteristic of the dopant material. The reference of the amount of use of a dopant is preferably from 0.001 to 50% by weight, more preferably from 0.05 to 20% by weight, and still more preferably from 0.1 to 10% by weight with respect to the total amount of a material for a light emitting layer. The amount of use within the above range is preferable, for example, from a viewpoint of being able to prevent a concentration quenching phenomenon.

Examples of a host material that can be used in combination with a compound represented by the above general formula (2) include a fused ring derivative such as anthracene or pyrene conventionally known as a luminous body, a bisstyryl derivative such as a bisstyrylanthracene derivative or a distyrylbenzene derivative, a tetraphenylbutadiene derivative, a cyclopentadiene derivative, a fluorene derivative, and a benzofluorene derivative.

<Electron Injection Layer and Electron Transport Layer in Organic Electroluminescent Element>

The electron injection layer 107 plays a role of efficiently injecting an electron migrating from the negative electrode 108 into the light emitting layer 105 or the electron transport layer 106. The electron transport layer 106 plays a role of efficiently transporting an electron injected from the negative electrode 108, or an electron injected from the negative electrode 108 through the electron injection layer 107 to the light emitting layer 105. The electron transport layer 106 and the electron injection layer 107 are each formed by laminating and mixing one or more kinds of electron transport/injection materials, or by a mixture of an electron transport/injection material and a polymer binder.

The electron injection/transport layer manages injection of an electron from a negative electrode and further manages transport of an electron, and desirably has a high electron injection efficiency and can efficiently transport an injected electron. For this purpose, a substance which has high electron affinity and large electron mobility, and further has excellent stability, and in which impurities serving as traps are not easily generated at the time of manufacturing and at the time of use, is preferable. However, when a transport balance between a hole and an electron is considered, in a case where the electron injection/transport layer mainly plays a role of efficiently preventing a hole coming from a positive electrode from flowing toward a negative electrode side without being recombined, even if electron transport ability is not so high, the electron injection/transport layer has an effect of enhancing a light emission efficiency equally to a material having high electron transport ability. Therefore, the electron injection/transport layer in the present embodiment may also include a function of a layer capable of efficiently preventing migration of a hole.

A material (electron transport material) for forming the electron transport layer 106 or the electron injection layer 107 can be arbitrarily selected for use from compounds conventionally used as electron transfer compounds in a photoconductive material, and known compounds that are used in an electron injection layer and an electron transport layer of an organic EL element.

A material used in an electron transport layer or an electron injection layer preferably includes at least one selected from a compound formed of an aromatic ring or a heteroaromatic ring including one or more kinds of atoms selected from carbon, hydrogen, oxygen, sulfur, silicon, and phosphorus atoms, a pyrrole derivative and a fused ring derivative thereof, and a metal complex having an electron-accepting nitrogen atom. Specific examples of the material include a fused ring-based aromatic ring derivative of naphthalene, anthracene, or the like, a styryl-based aromatic ring derivative represented by 4,4′-bis(diphenylethenyl)biphenyl, a perinone derivative, a coumarin derivative, a naphthalimide derivative, a quinone derivative such as anthraquinone or diphenoquinone, a phosphorus oxide derivative, a carbazole derivative, and an indole derivative. Examples of the metal complex having an electron-accepting nitrogen atom include a hydroxyazole complex such as a hydroxyphenyloxazole complex, an azomethine complex, a tropolone metal complex, a flavonol metal complex, and a benzoquinoline metal complex. These materials are used singly, but may also be used in a mixture with other materials.

Furthermore, specific examples of other electron transfer compounds include a pyridine derivative, a naphthalene derivative, an anthracene derivative, a phenanthroline derivative, a perinone derivative, a coumarin derivative, a naphthalimide derivative, an anthraquinone derivative, a diphenoquinone derivative, a diphenylquinone derivative, a perylene derivative, an oxadiazole derivative (1,3-bis[(4-t-butylphenyl)-1,3,4-oxadiazolyl]phenylene and the like), a thiophene derivative, a triazole derivative (N-naphthyl-2,5-diphenyl-1,3,4-triazole and the like), a thiadiazole derivative, a metal complex of an oxine derivative, a quinolinol-based metal complex, a quinoxaline derivative, a polymer of a quinoxaline derivative, a benzazole compound, a gallium complex, a pyrazole derivative, a perfluorinated phenylene derivative, a triazine derivative, a pyrazine derivative, a benzoquinoline derivative (2,2′-bis(benzo[h]quinolin-2-yl)-9,9′-spirobifluorene and the like), an imidazopyridine derivative, a borane derivative, a benzimidazole derivative (tris(N-phenylbenzimidazol-2-yl)benzene and the like), a benzoxazole derivative, a benzothiazole derivative, a quinoline derivative, an oligopyridine derivative such as terpyridine, a bipyridine derivative, a terpyridine derivative (1,3-bis(4′-(2,2′:6′2″-terpyridinyl))benzene and the like), a naphthyridine derivative (bis(1-naphthyl)-4-(1,8-naphthyridin-2-yl)phenylphosphine oxide and the like), an aldazine derivative, a carbazole derivative, an indole derivative, a phosphorus oxide derivative, and a bisstyryl derivative.

Furthermore, a metal complex having an electron-accepting nitrogen atom can be also used, and examples thereof include a quinolinol-based metal complex, a hydroxyazole complex such as a hydroxyphenyloxazole complex, an azomethine complex, a tropolone metal complex, a flavonol metal complex, and a benzoquinoline metal complex.

The materials described above are used singly, but may also be used in a mixture with other materials.

Among the above materials, a borane derivative, a pyridine derivative, a fluoranthene derivative, a BO-based derivative, an anthracene derivative, a benzofluorene derivative, a phosphine oxide derivative, a pyrimidine derivative, a carbazole derivative, a triazine derivative, a benzimidazole derivative, a phenanthroline derivative, a quinolinol-based metal complex are preferable.

<Borane Derivative>

The borane derivative is, for example, a compound represented by the following general formula (ETM-1), and specifically disclosed in JP 2007-27587 A.

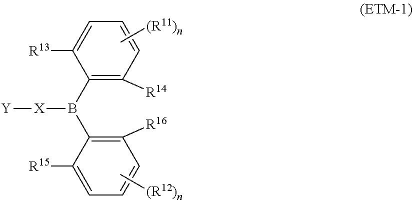

In the above formula (ETM-1), R¹¹ and R¹² each independently represent at least one of a hydrogen atom, an alkyl, an optionally substituted aryl, a substituted silyl, an optionally substituted nitrogen-containing heterocyclic ring, and a cyano, R¹³ to R¹⁶ each independently represent an optionally substituted alkyl, or an optionally substituted aryl, X represents an optionally substituted arylene, Y represents an optionally substituted aryl having 16 or fewer carbon atoms, a substituted boryl, or an optionally substituted carbazolyl, and n's each independently represent an integer of 0 to 3.

Among compounds represented by the above general formula (ETM-1), a compound represented by the following general formula (ETM-1-1) and a compound represented by the following general formula (ETM-1-2) are preferable.

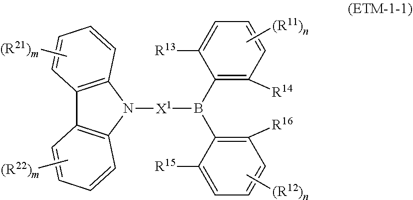

In formula (ETM-1-1), R¹¹ and R¹² each independently represent at least one of a hydrogen atom, an alkyl, an optionally substituted aryl, a substituted silyl, an optionally substituted nitrogen-containing heterocyclic ring, and a cyano, R¹³ to R¹⁶ each independently represent an optionally substituted alkyl, or an optionally substituted aryl, R²¹ and R²² each independently represent at least one of a hydrogen atom, an alkyl, an optionally substituted aryl, a substituted silyl, an optionally substituted nitrogen-containing heterocyclic ring, and a cyano, X¹ represents an optionally substituted arylene having 20 or fewer carbon atoms, n's each independently represent an integer of 0 to 3, and m's each independently represent an integer of 0 to 4.

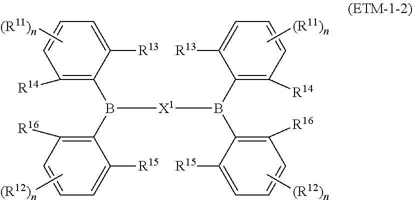

In formula (ETM-1-2), R¹¹ and R¹² each independently represent at least one of a hydrogen atom, an alkyl, an optionally substituted aryl, a substituted silyl, an optionally substituted nitrogen-containing heterocyclic ring, and cyano, R¹³ to R¹⁶ each independently represent an optionally substituted alkyl, or an optionally substituted aryl, X¹ represents an optionally substituted arylene having 20 or fewer carbon atoms, and n's each independently represent an integer of 0 to 3.

Specific examples of X¹ include divalent groups represented by the following formulas (X-1) to (X-9).

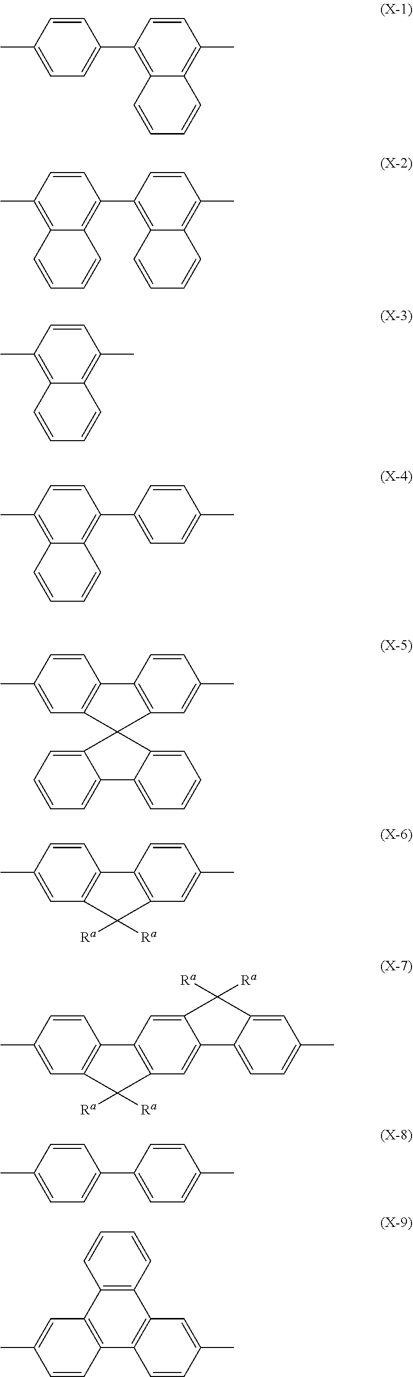

(In each formula, R^{a}'s each independently represent an alkyl group, or an optionally substituted phenyl group.)

Specific examples of this borane derivative include the following compounds.

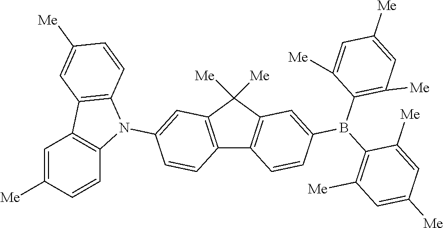 [SMILES: Cc1cc(C)c(B(c2ccc3c(c2)C(C)(C)c2cc(-n4c5ccc(C)cc5c5cc(C)ccc54)ccc2-3)c2c(C)cc(C)cc2C)c(C)c1]

This borane derivative can be manufactured using known raw materials and known synthesis methods.

<Pyridine Derivative>

A pyridine derivative is, for example, a compound represented by the following formula (ETM-2), and preferably a compound represented by formula (ETM-2-1) or (ETM-2-2).

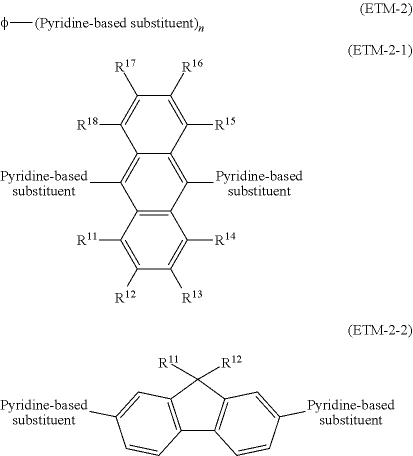

φ represents an n-valent aryl ring (preferably, an n-valent benzene ring, naphthalene ring, anthracene ring, fluorene ring, benzofluorene ring, phenalene ring, phenanthrene ring, or triphenylene ring), and n represents an integer of 1 to 4.

In the above formula (ETM-2-1), R¹¹ to R¹⁸ each independently represent a hydrogen atom, an alkyl (preferably, an alkyl having 1 to 24 carbon atoms), a cycloalkyl (preferably, a cycloalkyl having 3 to 12 carbon atoms), or an aryl (preferably, an aryl having 6 to 30 carbon atoms).

In the above formula (ETM-2-2), R¹¹ and R¹² each independently represent a hydrogen atom, an alkyl (preferably, an alkyl having 1 to 24 carbon atoms), a cycloalkyl (preferably, a cycloalkyl having 3 to 12 carbon atoms), or an aryl (preferably, an aryl having 6 to 30 carbon atoms), and R¹¹ and R¹² may be bonded to each other to form a ring.

In each formula, the “pyridine-based substituent” is any one of the following formulas (Py-1) to (Py-15), and the pyridine-based substituents may be each independently substituted by an alkyl having 1 to 4 carbon atoms. The pyridine-based substituent may be bonded to p, an anthracene ring, or a fluorene ring in each formula via a phenylene group or a naphthylene group.

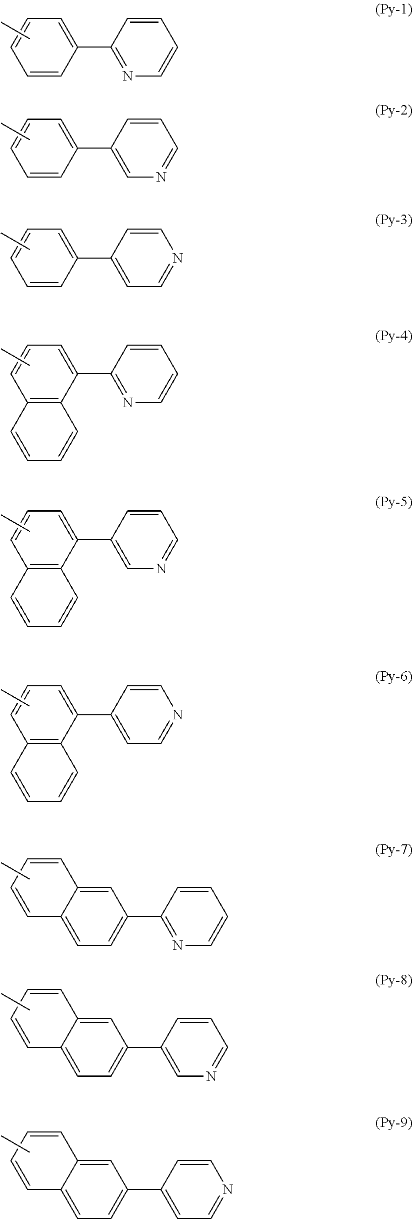

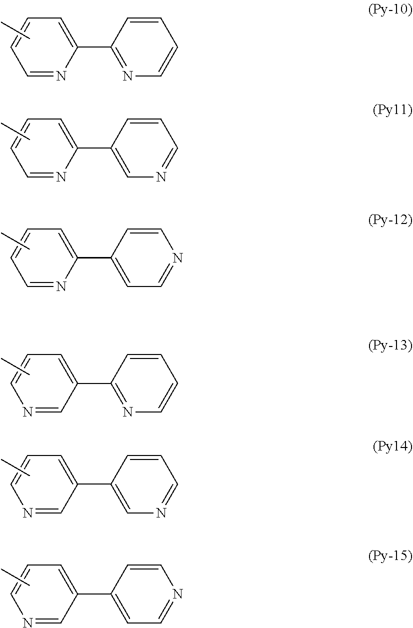

The pyridine-based substituent is any one of the above-formulas (Py-1) to (Py-15). However, among these formulas, the pyridine-based substituent is preferably any one of the following formulas (Py-21) to (Py-44).

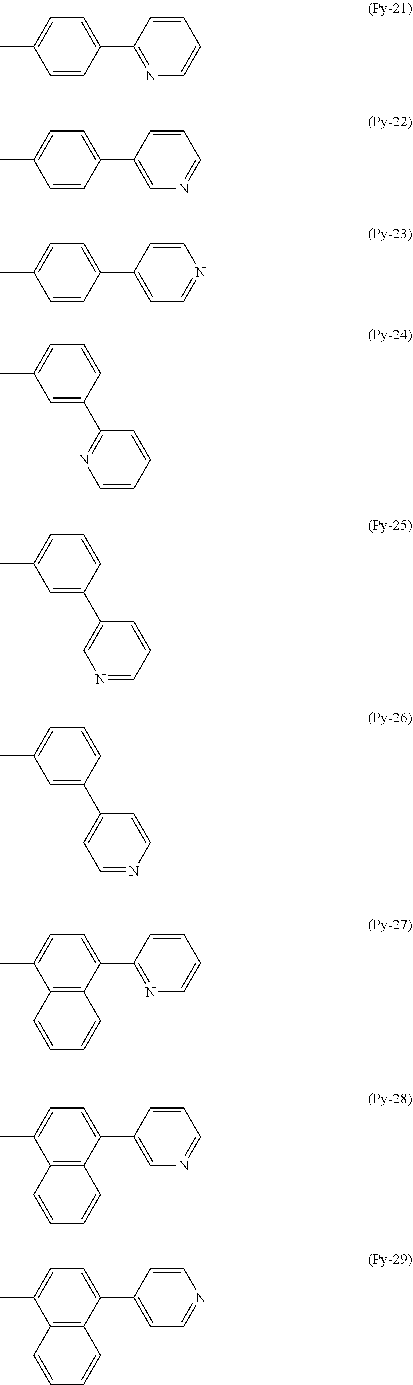

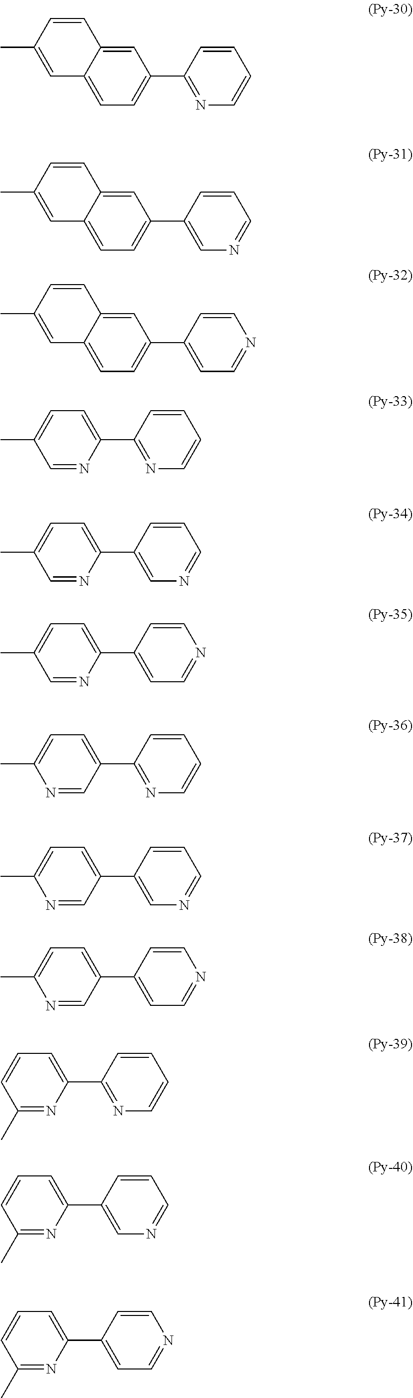

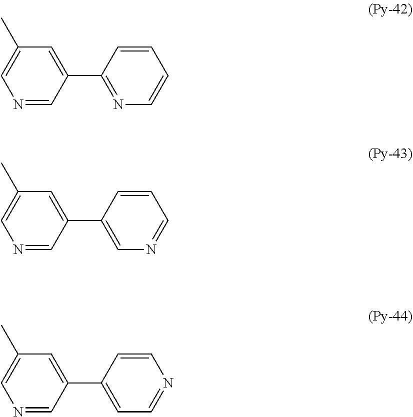

At least one hydrogen atom in each pyridine derivative may be substituted by a deuterium atom. One of the two “pyridine-based substituents” in the above formulas (ETM-2-1) and (ETM-2-2) may be substituted by an aryl.

The alkyl in R¹¹ to R¹⁸ may be either linear or branched, and examples thereof include a linear alkyl having 1 to 24 carbon atoms and a branched alkyl having 3 to 24 carbon atoms. A preferable “alkyl” is an alkyl having 1 to 18 carbon atoms (branched alkyl having 3 to 18 carbon atoms). A more preferable “alkyl” is an alkyl having 1 to 12 carbon atoms (branched alkyl having 3 to 12 carbon atoms). A still more preferable “alkyl” is an alkyl having 1 to 6 carbon atoms (branched alkyl having 3 to 6 carbon atoms). A particularly preferable “alkyl” is an alkyl having 1 to 4 carbon atoms (branched alkyl having 3 or 4 carbon atoms).

Specific examples of the “alkyl” include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl, 2-ethylbutyl, n-heptyl, 1-methylhexyl, n-octyl, t-octyl, 1-methylheptyl, 2-ethylhexyl, 2-propylpentyl, n-nonyl, 2,2-dimethylheptyl, 2,6-dimethyl-4-heptyl, 3,5,5-trimethylhexyl, n-decyl, n-undecyl, 1-methyldecyl, n-dodecyl, n-tridecyl, 1-hexylheptyl, n-tetradecyl, n-pentadecyl, n-hexadecyl, n-heptadecyl, n-octadecyl, and n-eicosyl.

As the alkyl having 1 to 4 carbon atoms by which the pyridine-based substituent is substituted, the above description of the alkyl can be cited.

Examples of the “cycloalkyl” in R¹¹ to R¹⁸ include a cycloalkyl having 3 to 12 carbon atoms. A preferable “cycloalkyl” is a cycloalkyl having 3 to 10 carbon atoms. A more preferable “cycloalkyl” is a cycloalkyl having 3 to 8 carbon atoms. A still more preferable “cycloalkyl” is a cycloalkyl having 3 to 6 carbon atoms.

Specific examples of the “cycloalkyl” include cyclopropyl, cyclobutyl, cyclopentyl, cyclohexyl, methylcyclopentyl, cycloheptyl, methylcyclohexyl, cyclooctyl, and dimethylcyclohexyl.

As the “aryl” in R¹¹ to R¹⁸, a preferable aryl is an aryl having 6 to 30 carbon atoms, a more preferable aryl is an aryl having 6 to 18 carbon atoms, a still more preferable aryl is an aryl having 6 to 14 carbon atoms, and a particularly preferable aryl is an aryl having 6 to 12 carbon atoms.

Specific examples of the “aryl having 6 to 30 carbon atoms” include phenyl which is a monocyclic aryl; (1-,2-)naphthyl which is a fused bicyclic aryl; acenaphthylene-(1-,3-,4-,5-)yl, a fluorene-(1-,2-,3-,4-,9-)yl, phenalene-(1-,2-)yl, and (1-,2-,3-,4-,9-)phenanthryl which are fused tricyclic aryls; triphenylene-(1-,2-)yl, pyrene-(1-,2-,4-)yl, and naphthacene-(1-,2-,5-)yl which are fused tetracyclic aryls; and perylene-(1-,2-,3-)yl and pentacene-(1-,2-,5-,6-)yl which are fused pentacyclic aryls.

Preferable examples of the “aryl having 6 to 30 carbon atoms” include a phenyl, a naphthyl, a phenanthryl, a chrysenyl, and a triphenylenyl. More preferable examples thereof include a phenyl, a 1-naphthyl, a 2-naphthyl, and a phenanthryl. Particularly preferable examples thereof include a phenyl, a 1-naphthyl, and a 2-naphthyl.

R¹¹ and R¹² in the above formula (ETM-2-2) may be bonded to each other to form a ring. As a result, cyclobutane, cyclopentane, cyclopentene, cyclopentadiene, cyclohexane, fluorene, indene, or the like may be spiro-bonded to a 5-membered ring of a fluorene skeleton.

Specific examples of this pyridine derivative include the following compounds.

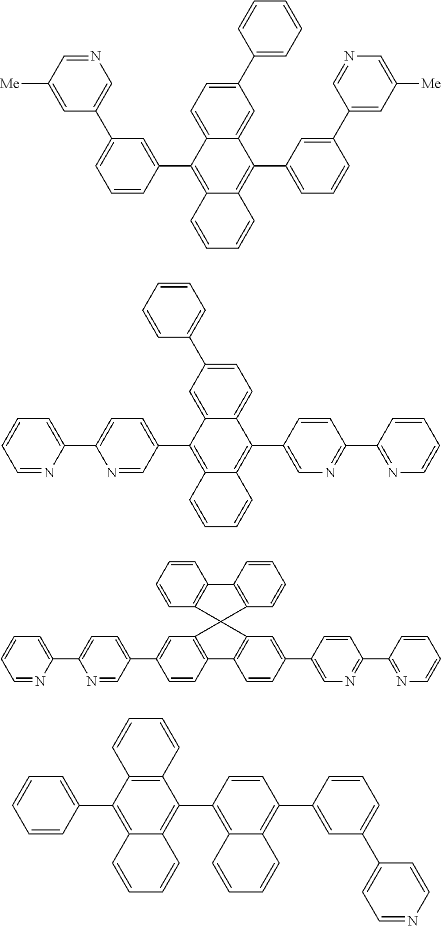

This pyridine derivative can be manufactured using known raw materials and known synthesis methods.

<Fluoranthene Derivative>

The fluoranthene derivative is, for example, a compound represented by the following general formula (ETM-3), and specifically disclosed in WO 2010/134352 A.

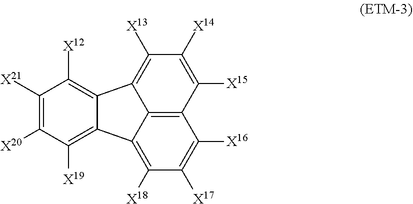

In the above formula (ETM-3), X¹² to X²¹ each represent a hydrogen atom, a halogen atom, a linear, branched or cyclic alkyl, a linear, branched or cyclic alkoxy, a substituted or unsubstituted aryl, or a substituted or unsubstituted heteroaryl.

Specific examples of this fluoranthene derivative include the following compounds.

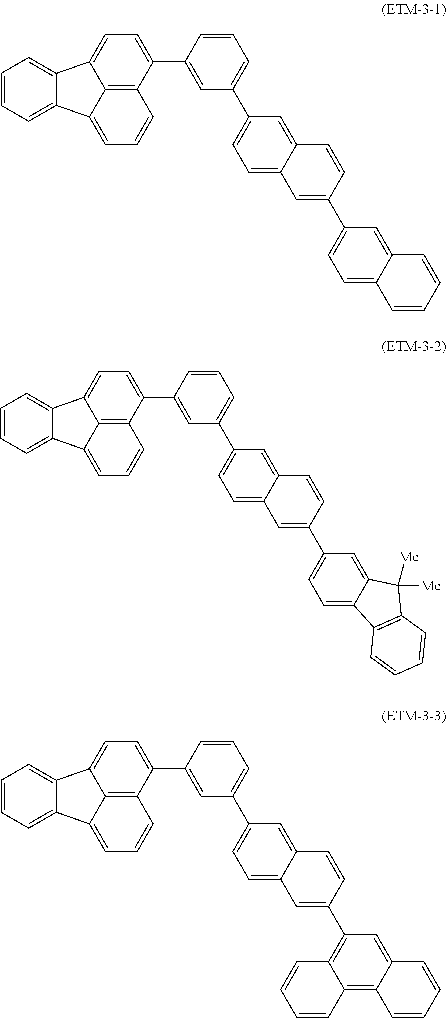

<BO-Based Derivative>

The BO-based derivative is, for example, a polycyclic aromatic compound represented by the following formula (ETM-4) or a polycyclic aromatic compound multimer having a plurality of structures represented by the following formula (ETM-4).

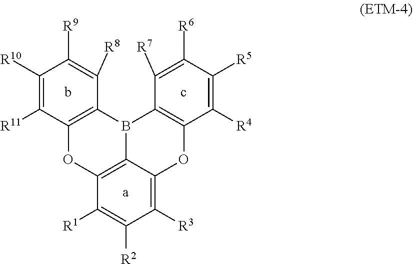

R¹ to R¹¹ each independently represent a hydrogen atom, an aryl, a heteroaryl, a diarylamino, a diheteroarylamino, an arylheteroarylamino, an alkyl, an alkoxy, or an aryloxy, and at least one hydrogen atom in these substituents may be substituted by an aryl, a heteroaryl, or an alkyl.

Adjacent groups among R¹ to R¹¹ may be bonded to each other to form an aryl ring or a heteroaryl ring together with the ring a, ring b, or ring c, and at least one hydrogen atom in the ring thus formed may be substituted by an aryl, a heteroaryl, a diarylamino, a diheteroarylamino, an arylheteroarylamino, an alkyl, an alkoxy, or an aryloxy, while at least one hydrogen atom in these substituents may be substituted by an aryl, a heteroaryl, or an alkyl.

At least one hydrogen atom in a compound or structure represented by formula (ETM-4) may be substituted by a halogen atom or a deuterium atom.

For description of a substituent and a form of ring formation in formula (ETM-4) or description of a multimer in which a plurality of structures of formula (ETM-4) are combined, the description of a compound or multimer thereof represented by the above general formula (1) or formula (1′) can be cited.

Specific examples of this BO-based derivative include the following compounds.

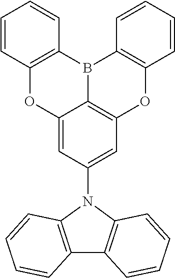 [SMILES: c1ccc2c(c1)Oc1cc(-n3c4ccccc4c4ccccc43)cc3c1B2c1ccccc1O3]

This BO-based derivative can be manufactured using known raw materials and known synthesis methods.

<Anthracene Derivative>

One of the anthracene derivatives is, for example, a compound represented by the following formula (ETM-5-1).

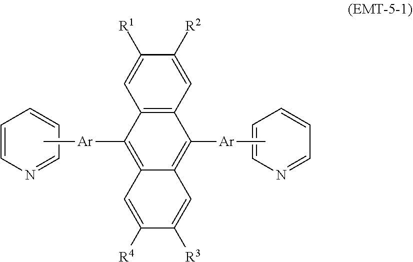

Ar's each independently represent a divalent benzene or naphthalene, R¹ to R⁴ each independently represent a hydrogen atom, an alkyl having 1 to 6 carbon atoms, a cycloalkyl having 3 to 6 carbon atoms, or an aryl having 6 to 20 carbon atoms.

Ar's can be each independently selected from a divalent benzene and naphthalene appropriately. Two Ar's may be different from or the same as each other, but are preferably the same from a viewpoint of easiness of synthesis of an anthracene derivative. Ar is bonded to pyridine to form “a moiety formed of Ar and pyridine”. For example, this moiety is bonded to anthracene as a group represented by any one of the following formulas (Py-1) to (Py-12).

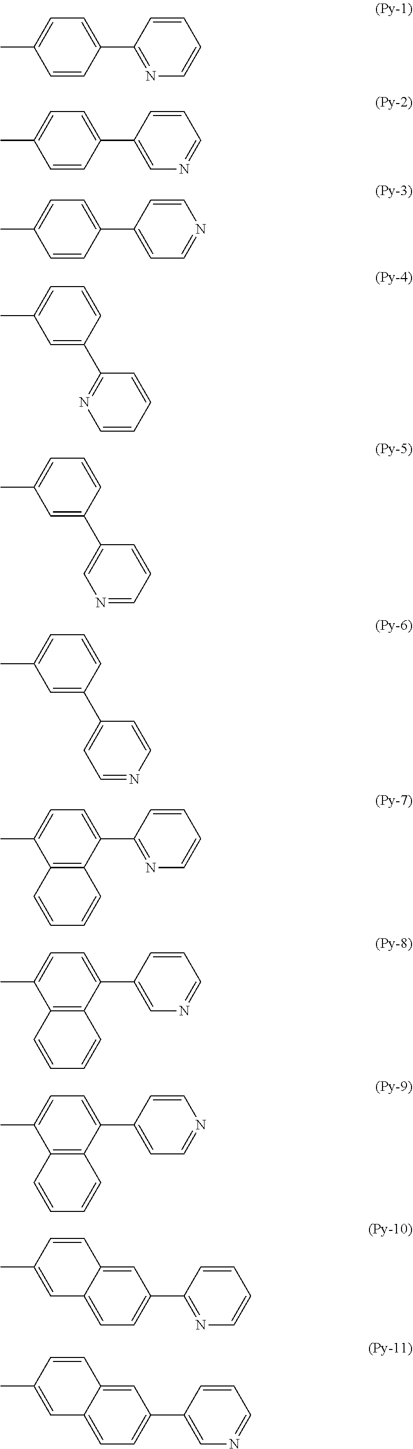

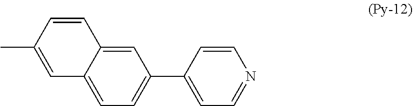 [SMILES: Cc1ccc2cc(-c3ccncc3)ccc2c1]

Among these groups, a group represented by any one of the above formulas (Py-1) to (Py-9) is preferable, and a group represented by any one of the above formulas (Py-1) to (Py-6) is more preferable. Two “moieties formed of Ar and pyridine” bonded to anthracene may have the same structure as or different structures from each other, but preferably have the same structure from a viewpoint of easiness of synthesis of an anthracene derivative. However, two “moieties formed of Ar and pyridine” preferably have the same structure or different structures from a viewpoint of element characteristics.

The alkyl having 1 to 6 carbon atoms in R¹ to R⁴ may be either linear or branched. That is, the alkyl having 1 to 6 carbon atoms is a linear alkyl having 1 to 6 carbon atoms or a branched alkyl having 3 to 6 carbon atoms. More preferably, the alkyl having 1 to 6 carbon atoms is an alkyl having 1 to 4 carbon atoms (branched alkyl having 3 to 4 carbon atoms). Specific examples thereof include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl, and 2-ethylbutyl. Methyl, ethyl, n-propyl, isopropyl, n-butyl, isobutyl, s-butyl, and t-butyl are preferable. Methyl, ethyl, and a t-butyl are more preferable.

Specific examples of the cycloalkyl having 3 to 6 carbon atoms in R¹ to R⁴ include cyclopropyl, cyclobutyl, cyclopentyl, cyclohexyl, methylcyclopentyl, cycloheptyl, methylcyclohexyl, cyclooctyl, and dimethylcyclohexyl.

For the aryl having 6 to 20 carbon atoms in R¹ to R⁴, an aryl having 6 to 16 carbon atoms is preferable, an aryl having 6 to 12 carbon atoms is more preferable, and an aryl having 6 to 10 carbon atoms is particularly preferable.

Specific examples of the “aryl having 6 to 20 carbon atoms” include phenyl, (o-, m-, p-) tolyl, (2,3-, 2,4-, 2,5-, 2,6-, 3,4-, 3,5-) xylyl, mesityl (2,4,6-trimethylphenyl), and (o-, m-, p-)cumenyl which are monocyclic aryls; (2-, 3-, 4-)biphenylyl which is a bicyclic aryl; (1-,2-)naphthyl which is a fused bicyclic aryl; terphenylyl (m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl) which is a tricyclic aryl; anthracene-(1-, 2-,9-)yl, acenaphthylene-(1-,3-,4-,5-)yl, fluorene-(1-, 2-,3-,4-,9-)yl, phenalene-(1-,2-)yl, and (1-,2-,3-, 4-,9-)phenanthryl which are fused tricyclic aryls; triphenylene-(1-,2-)yl, pyrene-(1-,2-,4-)yl, and tetracene-(1-,2-,5-)yl which are fused tetracyclic aryls; and perylene-(1-,2-,3-)yl which is a fused pentacyclic aryl.

The “aryl having 6 to 20 carbon atoms” is preferably a phenyl, a biphenylyl, a terphenylyl, or a naphthyl, more preferably a phenyl, a biphenylyl, a 1-naphthyl, a 2-naphthyl, or an m-terphenyl-5′-yl, still more preferably a phenyl, a biphenylyl, a 1-naphthyl, or a 2-naphthyl, and most preferably a phenyl.

One of the anthracene derivatives is, for example, a compound represented by the following formula (ETM-5-2).

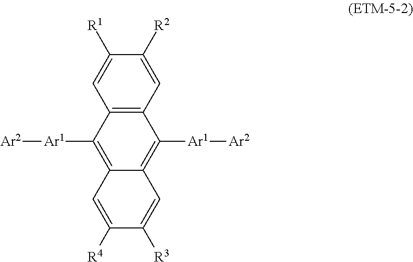

Ar¹'s each independently represent a single bond, a divalent benzene, naphthalene, anthracene, fluorene, or phenalene.

Ar²'s each independently represent an aryl having 6 to 20 carbon atoms. The same description as the “aryl having 6 to 20 carbon atoms” in the above formula (ETM-5-1) can be cited. An aryl having 6 to 16 carbon atoms is preferable, an aryl having 6 to 12 carbon atoms is more preferable, and an aryl having 6 to 10 carbon atoms is particularly preferable. Specific examples thereof include phenyl, biphenylyl, naphthyl, terphenylyl, anthracenyl, acenaphthylenyl, fluorenyl, phenalenyl, phenanthryl, triphenylenyl, pyrenyl, etracenyl, and perylenyl.

R¹ to R⁴ each independently represent a hydrogen atom, an alkyl having 1 to 6 carbon atoms, a cycloalkyl having 3 to 6 carbon atoms, or an aryl having 6 to 20 carbon atoms. The description in the above formula (ETM-5-1) can be cited.

Specific examples of these anthracene derivatives include the following compounds.

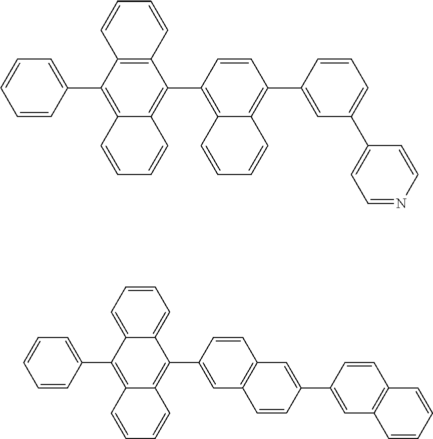

These anthracene derivatives can be manufactured using known raw materials and known synthesis methods.

<Benzofluorene Derivative>

The benzofluorene derivative is, for example, a compound represented by the following formula (ETM-6).

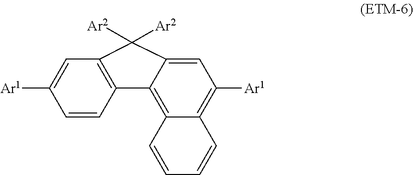

Ar¹'s each independently represent an aryl having 6 to 20 carbon atoms. The same description as the “aryl having 6 to 20 carbon atoms” in the above formula (ETM-5-1) can be cited. An aryl having 6 to 16 carbon atoms is preferable, an aryl having 6 to 12 carbon atoms is more preferable, and an aryl having 6 to 10 carbon atoms is particularly preferable. Specific examples thereof include phenyl, biphenylyl, naphthyl, terphenylyl, anthracenyl, acenaphthylenyl, fluorenyl, phenalenyl, phenanthryl, triphenylenyl, pyrenyl, etracenyl, and perylenyl.

Ar²'s each independently represent a hydrogen atom, an alkyl (preferably, an alkyl having 1 to 24 carbon atoms), a cycloalkyl (preferably, a cycloalkyl having 3 to 12 carbon atoms), or an aryl (preferably, an aryl having 6 to 30 carbon atoms), and two Ar²'s may be bonded to each other to form a ring.

The alkyl as Ar² may be either linear or branched, and examples thereof include a linear alkyl having 1 to 24 carbon atoms and a branched alkyl having 3 to 24 carbon atoms. A preferable “alkyl” is an alkyl having 1 to 18 carbon atoms (branched alkyl having 3 to 18 carbon atoms). A more preferable “alkyl” is an alkyl having 1 to 12 carbon atoms (branched alkyl having 3 to 12 carbon atoms). A still more preferable “alkyl” is an alkyl having 1 to 6 carbon atoms (branched alkyl having 3 to 6 carbon atoms). A particularly preferable “alkyl” is an alkyl having 1 to 4 carbon atoms (branched alkyl having 3 or 4 carbon atoms). Specific examples of the “alkyl” include methyl, ethyl, n-propyl, isopropyl, n-butyl, isobutyl, s-butyl, t-butyl, n-pentyl, isopentyl, neopentyl, t-pentyl, n-hexyl, 1-methylpentyl, 4-methyl-2-pentyl, 3,3-dimethylbutyl, 2-ethylbutyl, n-heptyl, and 1-methylhexyl.

Examples of the “cycloalkyl” in Ar² include a cycloalkyl having 3 to 12 carbon atoms. A preferable “cycloalkyl” is a cycloalkyl having 3 to 10 carbon atoms. A more preferable “cycloalkyl” is a cycloalkyl having 3 to 8 carbon atoms. A still more preferable “cycloalkyl” is a cycloalkyl having 3 to 6 carbon atoms. Specific examples of the “cycloalkyl” include cyclopropyl, cyclobutyl, cyclopentyl, cyclohexyl, methylcyclopentyl, cycloheptyl, methylcyclohexyl, cyclooctyl, and dimethylcyclohexyl.

As the “aryl” in Ar², a preferable aryl is an aryl having 6 to 30 carbon atoms, a more preferable aryl is an aryl having 6 to 18 carbon atoms, a still more preferable aryl is an aryl having 6 to 14 carbon atoms, and a particularly preferable aryl is an aryl having 6 to 12 carbon atoms.

Specific examples of the “aryl having 6 to 30 carbon atoms” include phenyl, naphthyl, acenaphthylenyl, fluorenyl, phenalenyl, phenanthryl, triphenylenyl, pyrenyl, naphthacenyl, perylenyl, and pentacenyl.

Two Ar²'s may be bonded to each other to form a ring. As a result, cyclobutane, cyclopentane, cyclopentene, cyclopentadiene, cyclohexane, fluorene, indene, or the like may be spiro-bonded to a 5-membered ring of a fluorene skeleton.

Specific examples of this benzofluorene derivative include the following compounds.

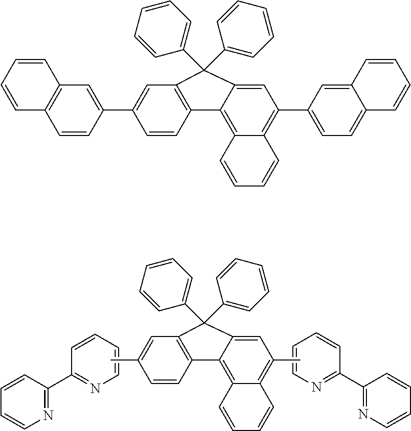

This benzofluorene derivative can be manufactured using known raw materials and known synthesis methods.

<Phosphine Oxide Derivative>

The phosphine oxide derivative is, for example, a compound represented by the following formula (ETM-7-1). Details are also described in WO 2013/079217 A.

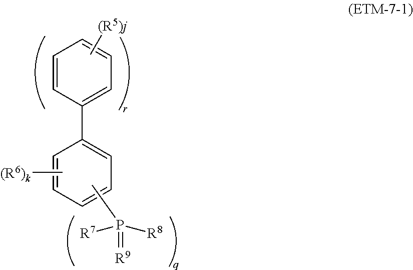

R⁵ represents a substituted or unsubstituted alkyl having 1 to 20 carbon atoms, a substituted or unsubstituted aryl having 6 to 20 carbon atoms, or a substituted or unsubstituted heteroaryl having 5 to 20 carbon atoms,

R⁶ represents CN, a substituted or unsubstituted alkyl having 1 to 20 carbon atoms, a substituted or unsubstituted heteroalkyl having 1 to 20 carbon atoms, a substituted or unsubstituted aryl having 6 to 20 carbon atoms, a substituted or unsubstituted heteroaryl having 5 to 20 carbon atoms, a substituted or unsubstituted alkoxy having 1 to 20 carbon atoms, or a substituted or unsubstituted aryloxy having 6 to 20 carbon atoms,

R⁷ and R⁸ each independently represent a substituted or unsubstituted aryl having 6 to 20 carbon atoms or a substituted or unsubstituted heteroaryl having 5 to 20 carbon atoms,

R⁹ represents an oxygen atom or a sulfur atom,

j represents 0 or 1, k represents 0 or 1, r represents an integer of 0 to 4, and q represents an integer of 1 to 3.

The phosphine oxide derivative may be, for example, a compound represented by the following formula (ETM-7-2).

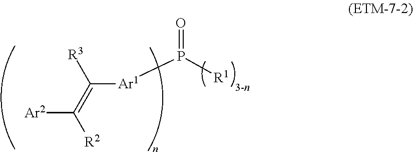

R¹ to R³ may be the same as or different from each other and are selected from a hydrogen atom, an alkyl group, a cycloalkyl group, an aralkyl group, an alkenyl group, a cycloalkenyl group, an alkynyl group, an alkoxy group, an alkylthio group, an aryl ether group, an aryl thioether group, an aryl group, a heterocyclic group, a halogen atom, a cyano group, an aldehyde group, a carbonyl group, a carboxyl group, an amino group, a nitro group, a silyl group, and a fused ring formed with an adjacent substituent.

Ar¹'s may be the same as or different from each other, and represents an arylene group or a heteroarylene group. Ar²'s may be the same as or different from each other, and represents an aryl group or a heteroaryl group. However, at least one of Ar¹ and Ar² has a substituent or forms a fused ring with an adjacent substituent. n represents an integer of 0 to 3. When n is 0, no unsaturated structure portion is present. When n is 3, R¹ is not present.

Among these substituents, the alkyl group represents a saturated aliphatic hydrocarbon group such as a methyl group, an ethyl group, a propyl group, or a butyl group. This saturated aliphatic hydrocarbon group may be unsubstituted or substituted. The substituent in a case of being substituted is not particularly limited, and examples thereof include an alkyl group, an aryl group, and a heterocyclic group, and this point is also common to the following description. The number of carbon atoms in the alkyl group is not particularly limited, but is usually in a range of 1 to 20 from a viewpoint of availability and cost.

The cycloalkyl group represents a saturated alicyclic hydrocarbon group such as a cyclopropyl, a cyclohexyl, a norbornyl, or an adamanty. This saturated alicyclic hydrocarbon group may be unsubstituted or substituted. The carbon number of the alkyl group moiety is not particularly limited, but is usually in a range of 3 to 20.

Furthermore, the aralkyl group represents an aromatic hydrocarbon group via an aliphatic hydrocarbon, such as a benzyl group or a phenylethyl group. Both the aliphatic hydrocarbon and the aromatic hydrocarbon may be unsubstituted or substituted. The carbon number of the aliphatic moiety is not particularly limited, but is usually in a range of 1 to 20.

The alkenyl group represents an unsaturated aliphatic hydrocarbon group containing a double bond, such as a vinyl group, an allyl group, or a butadienyl group. This unsaturated aliphatic hydrocarbon group may be unsubstituted or substituted. The carbon number of the alkenyl group is not particularly limited, but is usually in a range of 2 to 20.

The cycloalkenyl group represents an unsaturated alicyclic hydrocarbon group containing a double bond, such as a cyclopentenyl group, a cyclopentadienyl group, or a cyclohexene group. This unsaturated alicyclic hydrocarbon group may be unsubstituted or substituted.

The alkynyl group represents an unsaturated aliphatic hydrocarbon group containing a triple bond, such as an acetylenyl group. This unsaturated aliphatic hydrocarbon group may be unsubstituted or substituted. The carbon number of the alkynyl group is not particularly limited, but is usually in a range of 2 to 20.

The alkoxy group represents an aliphatic hydrocarbon group via an ether bond, such as a methoxy group. The aliphatic hydrocarbon group may be unsubstituted or substituted. The carbon number of the alkoxy group is not particularly limited, but is usually in a range of 1 to 20.

The alkylthio group is a group in which an oxygen atom of an ether bond of an alkoxy group is substituted by a sulfur atom.

The aryl ether group represents an aromatic hydrocarbon group via an ether bond, such as a phenoxy group. The aromatic hydrocarbon group may be unsubstituted or substituted. The carbon number of the aryl ether group is not particularly limited, but is usually in a range of 6 to 40.

The aryl thioether group is a group in which an oxygen atom of an ether bond of an aryl ether group is substituted by a sulfur atom.

Furthermore, the aryl group represents an aromatic hydrocarbon group such as a phenyl group, a naphthyl group, a biphenylyl group, a phenanthryl group, a terphenylyl group, or a pyrenyl group. The aryl group may be unsubstituted or substituted. The carbon number of the aryl group is not particularly limited, but is usually in a range of 6 to 40.

Furthermore, the heterocyclic group represents a cyclic structural group having an atom other than a carbon atom, such as a furanyl group, a thiophenyl group, an oxazolyl group, a pyridyl group, a quinolinyl group, or a carbazolyl group. This cyclic structural group may be unsubstituted or substituted. The carbon number of the heterocyclic group is not particularly limited, but is usually in a range of 2 to 30.

Halogen refers to fluorine, chlorine, bromine, and iodine.

The aldehyde group, the carbonyl group, and the amino group can include those substituted by an aliphatic hydrocarbon, an alicyclic hydrocarbon, an aromatic hydrocarbon, a heterocyclic ring, or the like.

Furthermore, the aliphatic hydrocarbon, the alicyclic hydrocarbon, the aromatic hydrocarbon, and the heterocyclic ring may be unsubstituted or substituted.

The silyl group represents, for example, a silicon compound group such as a trimethylsilyl group. This silicon compound group may be unsubstituted or substituted. The number of carbon atoms of the silyl group is not particularly limited, but is usually in a range of 3 to 20. The number of silicon atoms is usually 1 to 6.

The fused ring formed with an adjacent substituent is, for example, a conjugated or unconjugated fused ring formed between Ar¹ and R², Ar¹ and R³, Ar² and R², Ar² and R³, R² and R³, or Ar¹ and Ar². Here, when n is 1, two R¹'s may form a conjugated or nonconjugated fused ring. These fused rings may contain a nitrogen atom, an oxygen atom, or a sulfur atom in the ring structure, or may be fused with another ring.

Specific examples of this phosphine oxide derivative include the following compounds.

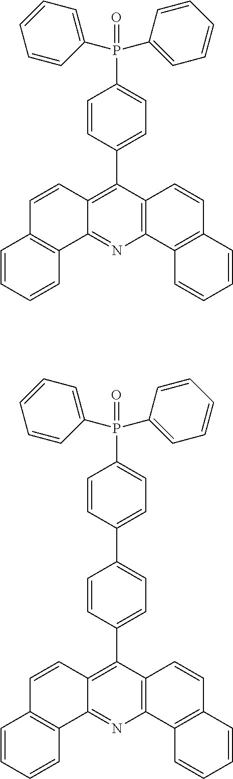 [SMILES: O=P(c1ccccc1)(c1ccccc1)c1ccc(-c2c3ccc4ccccc4c3nc3c2ccc2ccccc23)cc1 ; O=P(c1ccccc1)(c1ccccc1)c1ccc(-c2ccc(-c3c4ccc5ccccc5c4nc4c3ccc3ccccc34)cc2)cc1]

This phosphine oxide derivative can be manufactured using known raw materials and known synthesis methods.

<Pyrimidine Derivative>

The pyrimidine derivative is, for example, a compound represented by the following formula (ETM-8), and preferably a compound represented by the following formula (ETM-8-1). Details are also described in WO 2011/021689 A.

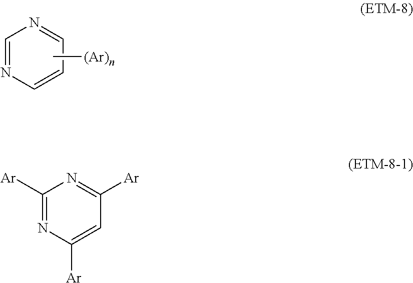

Ar's each independently represent an optionally substituted aryl or an optionally substituted heteroaryl. n represents an integer of 1 to 4, preferably an integer of 1 to 3, and more preferably 2 or 3.

Examples of the “aryl” as the “optionally substituted aryl” include an aryl having 6 to 30 carbon atoms. An aryl having 6 to 24 carbon atoms is preferable, an aryl having 6 to 20 carbon atoms is more preferable, and an aryl having 6 to 12 carbon atoms is still more preferable.

Specific examples of the “aryl” include phenyl which is a monocyclic aryl; (2-, 3-, 4-)biphenylyl which is a bicyclic aryl; (1-,2-)naphthyl which is a fused bicyclic aryl; terphenylyl (m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl) which is a tricyclic aryl; acenaphthylene-(1-,3-,4-,5-)yl, fluorene-(1-,2-,3-,4-,9-)yl, phenalene-(1-,2-)yl, and (1-,2-,3-,4-,9-)phenanthryl which are fused tricyclic aryls; quaterphenylyl-(5′-phenyl-m-terphenyl-2-yl, 5′-phenyl-m-terphenyl-3-yl, 5′-phenyl-m-terphenyl-4-yl, m-quaterphenylyl) which is a tetracyclic aryl; triphenylene-(1-,2-)yl, pyrene-(1-,2-,4-)yl, and naphthacene-(1-,2-,5-)yl which are fused tetracyclic aryls; and perylene-(1-,2-,3-)yl and pentacene-(1-,2-,5-,6-)yl which are fused pentacyclic aryls.

Examples of the “heteroaryl” as the “optionally substituted heteroaryl” include a heteroaryl having 2 to 30 carbon atoms. A heteroaryl having 2 to 25 carbon atoms is preferable, a heteroaryl having 2 to 20 carbon atoms is more preferable, a heteroaryl having 2 to 15 carbon atoms is still more preferable, and a heteroaryl having 2 to 10 carbon atoms is particularly preferable. In addition, examples of the “heteroaryl” include a heterocyclic ring containing 1 to 5 heteroatoms selected from an oxygen atom, a sulfur atom, and a nitrogen atom in addition to a carbon atom as a ring-constituting atom.

Specific examples of the “heteroaryl” include furyl, thienyl, pyrrolyl, oxazolyl, isoxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl, benzofuranyl, isobenzofuranyl, benzo[b]thienyl, indolyl, isoindolyl, 1H-indazolyl, benzoimidazolyl, benzoxazolyl, benzothiazolyl, 1H-benzotriazolyl, quinolyl, isoquinolyl, cinnolyl, quinazolyl, quinoxalinyl, phthalazinyl, naphthyridinyl, purinyl, pteridinyl, carbazolyl, acridinyl, phenoxazinyl, phenothiazinyl, phenazinyl, phenoxathiinyl, thianthrenyl, and indolizinyl.

The above aryl and heteroaryl may be substituted, and may be each substituted by, for example, the above aryl or heteroaryl.

Specific examples of this pyrimidine derivative include the following compounds.

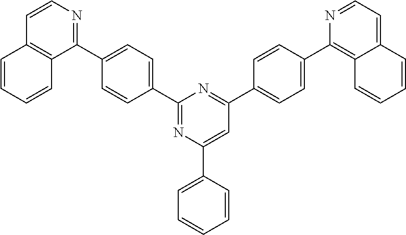 [SMILES: c1ccc(-c2cc(-c3ccc(-c4nccc5ccccc45)cc3)nc(-c3ccc(-c4nccc5ccccc45)cc3)n2)cc1]

This pyrimidine derivative can be manufactured using known raw materials and known synthesis methods.

<Carbazole Derivative>

The carbazole derivative is, for example, a compound represented by the following formula (ETM-9), or a multimer obtained by bonding a plurality of the compounds with a single bond or the like. Details are described in US 2014/0197386 A.

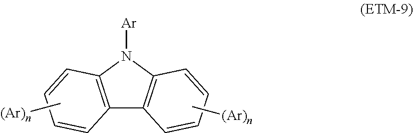

Ar's each independently represent an optionally substituted aryl or an optionally substituted heteroaryl. n's each independently represent an integer of 0 to 4, preferably an integer of 0 to 3, and more preferably 0 or 1.

Examples of the “aryl” as the “optionally substituted aryl” include an aryl having 6 to 30 carbon atoms. An aryl having 6 to 24 carbon atoms is preferable, an aryl having 6 to 20 carbon atoms is more preferable, and an aryl having 6 to 12 carbon atoms is still more preferable.

Specific examples of the “aryl” include phenyl which is a monocyclic aryl; (2-, 3-, 4-)biphenylyl which is a bicyclic aryl; (1-,2-)naphthyl which is a fused bicyclic aryl; terphenylyl (m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl) which is a tricyclic aryl; acenaphthylene-(1-,3-,4-,5-)yl, fluorene-(1-,2-,3-,4-,9-)yl, phenalene-(1-,2-)yl, and (1-,2-,3-,4-,9-)phenanthryl which are fused tricyclic aryls; quaterphenylyl-(5′-phenyl-m-terphenyl-2-yl, 5′-phenyl-m-terphenyl-3-yl, 5′-phenyl-m-terphenyl-4-yl, m-quaterphenylyl) which is a tetracyclic aryl; triphenylene-(1-,2-)yl, pyrene-(1-,2-,4-)yl, and naphthacene-(1-,2-,5-)yl which are fused tetracyclic aryls; and perylene-(1-,2-,3-)yl and pentacene-(1-,2-,5-,6-)yl which are fused pentacyclic aryls.

Examples of the “heteroaryl” as the “optionally substituted heteroaryl” include a heteroaryl having 2 to 30 carbon atoms. A heteroaryl having 2 to 25 carbon atoms is preferable, a heteroaryl having 2 to 20 carbon atoms is more preferable, a heteroaryl having 2 to 15 carbon atoms is still more preferable, and a heteroaryl having 2 to 10 carbon atoms is particularly preferable. In addition, examples of the “heteroaryl” include a heterocyclic ring containing 1 to 5 heteroatoms selected from an oxygen atom, a sulfur atom, and a nitrogen atom in addition to a carbon atom as a ring-constituting atom.

Specific examples of the “heteroaryl” include furyl, thienyl, pyrrolyl, oxazolyl, isoxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl, benzofuranyl, isobenzofuranyl, benzo[b]thienyl, indolyl, isoindolyl, 1H-indazolyl, benzoimidazolyl, benzoxazolyl, benzothiazolyl, 1H-benzotriazolyl, quinolyl, isoquinolyl, cinnolyl, quinazolyl, quinoxalinyl, phthalazinyl, naphthyridinyl, purinyl, pteridinyl, carbazolyl, acridinyl, phenoxazinyl, phenothiazinyl, phenazinyl, phenoxathiinyl, thianthrenyl, and indolizinyl.

The above aryl and heteroaryl may be substituted, and may be each substituted by, for example, the above aryl or heteroaryl.

The carbazole derivative may be a multimer obtained by bonding a plurality of compounds represented by the above formula (ETM-9) with a single bond or the like. In this case, the compounds may be bonded with an aryl ring (preferably, a polyvalent benzene ring, naphthalene ring, anthracene ring, fluorene ring, benzofluorene ring, phenalene ring, phenanthrene ring or triphenylene ring) in addition to a single bond.

Specific examples of this carbazole derivative include the following compounds.

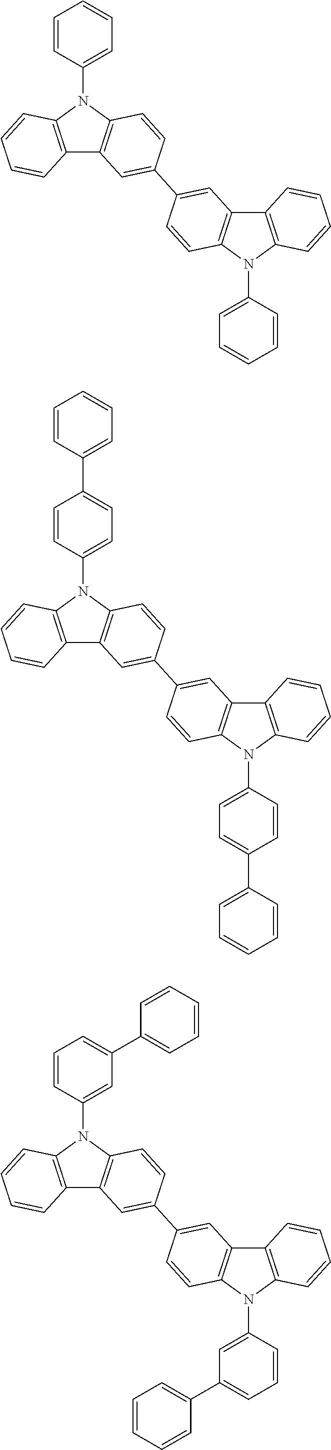 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

This carbazole derivative can be manufactured using known raw materials and known synthesis methods.

<Triazine Derivative>

The triazine derivative is, for example, a compound represented by the following formula (ETM-10), and preferably a compound represented by the following formula (ETM-10-1). Details are described in US 2011/0156013 A.

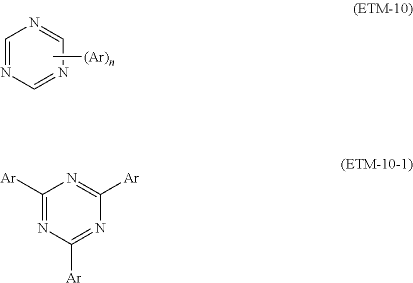

Ar's each independently represent an optionally substituted aryl or an optionally substituted heteroaryl. n represents an integer of 1 to 4, preferably 1 to 3, more preferably 2 or 3.

Examples of the “aryl” as the “optionally substituted aryl” include an aryl having 6 to 30 carbon atoms. An aryl having 6 to 24 carbon atoms is preferable, an aryl having 6 to 20 carbon atoms is more preferable, and an aryl having 6 to 12 carbon atoms is still more preferable.

Specific examples of the “aryl” include phenyl which is a monocyclic aryl; (2-, 3-, 4-)biphenylyl which is a bicyclic aryl; (1-,2-)naphthyl which is a fused bicyclic aryl; terphenylyl (m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl) which is a tricyclic aryl; acenaphthylene-(1-,3-,4-,5-)yl, fluorene-(1-,2-,3-,4-,9-)yl, phenalene-(1-,2-)yl, and (1-,2-,3-,4-,9-)phenanthryl which are fused tricyclic aryls; quaterphenylyl-(5′-phenyl-m-terphenyl-2-yl, 5′-phenyl-m-terphenyl-3-yl, 5′-phenyl-m-terphenyl-4-yl, m-quaterphenylyl) which is a tetracyclic aryl; triphenylene-(1-,2-)yl, pyrene-(1-,2-,4-)yl, and naphthacene-(1-,2-,5-)yl which are fused tetracyclic aryls; and perylene-(1-,2-,3-)yl and pentacene-(1-,2-,5-,6-)yl which are fused pentacyclic aryls.

Examples of the “heteroaryl” as the “optionally substituted heteroaryl” include a heteroaryl having 2 to 30 carbon atoms. A heteroaryl having 2 to 25 carbon atoms is preferable, a heteroaryl having 2 to 20 carbon atoms is more preferable, a heteroaryl having 2 to 15 carbon atoms is still more preferable, and a heteroaryl having 2 to 10 carbon atoms is particularly preferable. In addition, examples of the “heteroaryl” include a heterocyclic ring containing 1 to 5 heteroatoms selected from an oxygen atom, a sulfur atom, and a nitrogen atom in addition to a carbon atom as a ring-constituting atom.

Specific examples of the “heteroaryl” include furyl, thienyl, pyrrolyl, oxazolyl, isoxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl, benzofuranyl, isobenzofuranyl, benzo[b]thienyl, indolyl, isoindolyl, 1H-indazolyl, benzoimidazolyl, benzoxazolyl, benzothiazolyl, 1H-benzotriazolyl, quinolyl, isoquinolyl, cinnolyl, quinazolyl, quinoxalinyl, phthalazinyl, naphthyridinyl, purinyl, pteridinyl, carbazolyl, acridinyl, phenoxazinyl, phenothiazinyl, phenazinyl, phenoxathiinyl, thianthrenyl, and indolizinyl.

The above aryl and heteroaryl may be substituted, and may be each substituted by, for example, the above aryl or heteroaryl.

Specific examples of this triazine derivative include the following compounds.

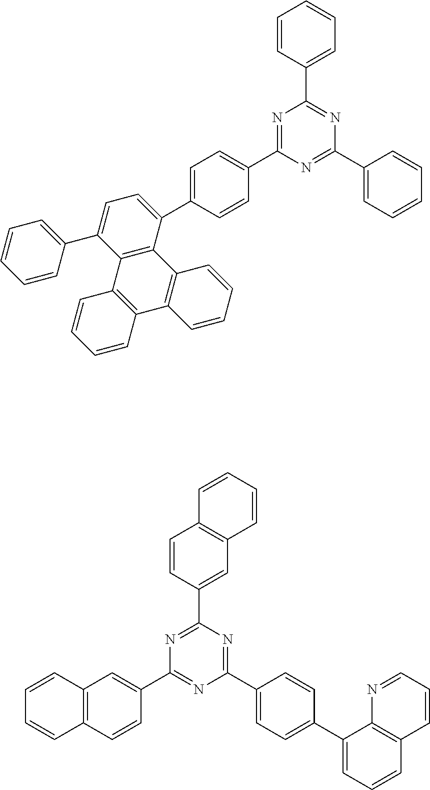 [SMILES: c1ccc(-c2nc(-c3ccccc3)nc(-c3ccc(-c4ccc(-c5ccccc5)c5c6ccccc6c6ccccc6c45)cc3)n2)cc1 ; c1ccc2cc(-c3nc(-c4ccc(-c5cccc6cccnc56)cc4)nc(-c4ccc5ccccc5c4)n3)ccc2c1]

This triazine derivative can be manufactured using known raw materials and known synthesis methods.

<Benzimidazole Derivative>

The benzimidazole derivative is, for example, a compound represented by the following formula (ETM-11).

ϕ-(Benzimidazole-based substituent)ₙ (ETM-11)

φ represents an n-valent aryl ring (preferably, an n-valent benzene ring, naphthalene ring, anthracene ring, fluorene ring, benzofluorene ring, phenalene ring, phenanthrene ring, or triphenylene ring), and n represents an integer of 1 to 4. A “benzimidazole-based substituent” is a substituent in which the pyridyl group in the “pyridine-based substituent” in the formulas (ETM-2), (ETM-2-1), and (ETM-2-2) is substituted by a benzimidazole group, and at least one hydrogen atom in the benzimidazole derivative may be substituted by a deuterium atom.

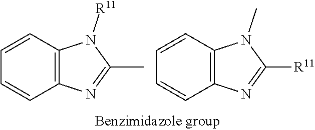

R¹¹ in the above benzimidazole represents a hydrogen atom, an alkyl having 1 to 24 carbon atoms, a cycloalkyl having 3 to 12 carbon atoms, or an aryl having 6 to 30 carbon atoms. The description of R¹¹ in the above formulas (ETM-2-1), and (ETM-2-2) can be cited.

Furthermore, y is preferably an anthracene ring or a fluorene ring. For the structure in this case, the description of the above formula (ETM-2-1) or (ETM-2-2) can be cited. For R¹ to R¹⁸ in each formula, the description of the above formula (ETM-2-1) or (ETM-2-2) can be cited. In the above formula (ETM-2-1) or (ETM-2-2), a form in which two pyridine-based substituents are bonded has been described. However, when these substituents are substituted by benzimidazole-based substituents, both the pyridine-based substituents may be substituted by benzimidazole-based substituents (that is, n=2), or one of the pyridine-based substituents may be substituted by a benzimidazole-based substituent and the other pyridine-based substituent may be substituted by any one of R¹ to R¹⁸ (that is, n=1). Furthermore, for example, at least one of R¹ to R¹⁸ in the above formula (ETM-2-1) may be substituted by a benzimidazole-based substituent and the “pyridine-based substituent” may be substituted by any one of R¹¹ to R¹⁸.

Specific examples of this benzimidazole derivative include 1-phenyl-2-(4-(10-phenylanthracen-9-yl)phenyl)-1H-benzo[d]imidazole, 2-(4-(10-(naphthalen-2-yl)anthracen-9-yl)phenyl)-1-phenyl-1H-benzo[d]imidazole, 2-(3-(10-(naphthalen-2-yl)anthracen-9-yl)phenyl)-1-phenyl-1H-benzo[d]imidazole, 5-(10-(naphthlen-2-yl)anthracen-9-yl)-1,2-diphenyl-1H-benzo[d]imidazole, 1-(4-(10-(naphthalen-2-yl)anthracen-9-yl)phenyl)-2-phenyl-1H-benzo[d]imidazole, 2-(4-(9,10-di(naphthalen-2-yl)anthracen-2-yl)phenyl)-1-phenyl-1H-benzo[d]imidazole, 1-(4-(9,10-di(naphthalen-2-yl)anthracen-2-yl)phenyl)-2-phenyl-1H-benzo[d]imidazole, and 5-(9,10-di(naphthalen-2-yl)anthracen-2-yl)-1,2-diphenyl-1H-benzo[d]imidazole.

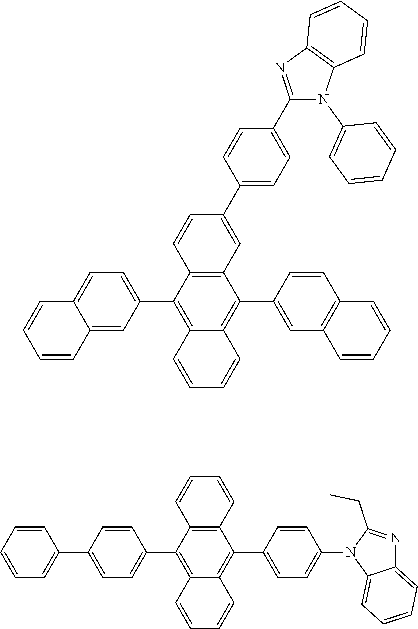 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3ccc(-c4ccccc4)cc3)c3ccccc23)cc1]

This benzimidazole derivative can be manufactured using known raw materials and known synthesis methods.

<Phenanthroline Derivative>

The phenanthroline derivative is, for example, a compound represented by the following formula (ETM-12) or (ETM-12-1). Details are described in WO 2006/021982 A.

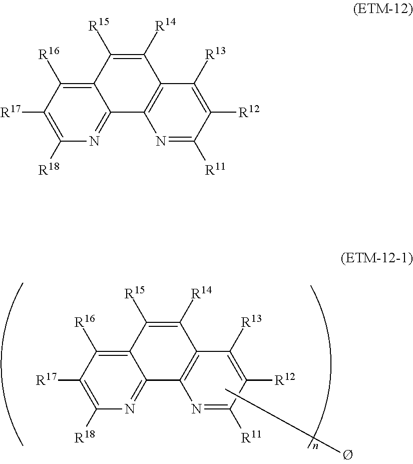

φ represents an n-valent aryl ring (preferably, an n-valent benzene ring, naphthalene ring, anthracene ring, fluorene ring, benzofluorene ring, phenalene ring, phenanthrene ring, or triphenylene ring), and n represents an integer of 1 to 4.

In each formula, R¹¹ to R¹⁸ each independently represent a hydrogen atom, an alkyl (preferably, an alkyl having 1 to 24 carbon atoms), a cycloalkyl (preferably, a cycloalkyl having 3 to 12 carbon atoms), or an aryl (preferably, an aryl having 6 to 30 carbon atoms). In the above formula (ETM-12-1), any one of R¹¹ to R¹⁸ is bonded to p which is an aryl ring.

At least one hydrogen atom in each phenanthroline derivative may be substituted by a deuterium atom.

For the alkyl, cycloalkyl, and aryl in R¹¹ to R¹⁸, the description of R^{1′} to R¹⁸ in the above formula (ETM-2) can be cited. In addition to the above examples, examples of the φ include those having the following structural formulas. Note that R's in the following structural formulas each independently represent a hydrogen atom, a methyl, an ethyl, an isopropyl, a cyclohexyl, a phenyl, a 1-naphthyl, a 2-naphthyl, a biphenylyl, or a terphenylyl.

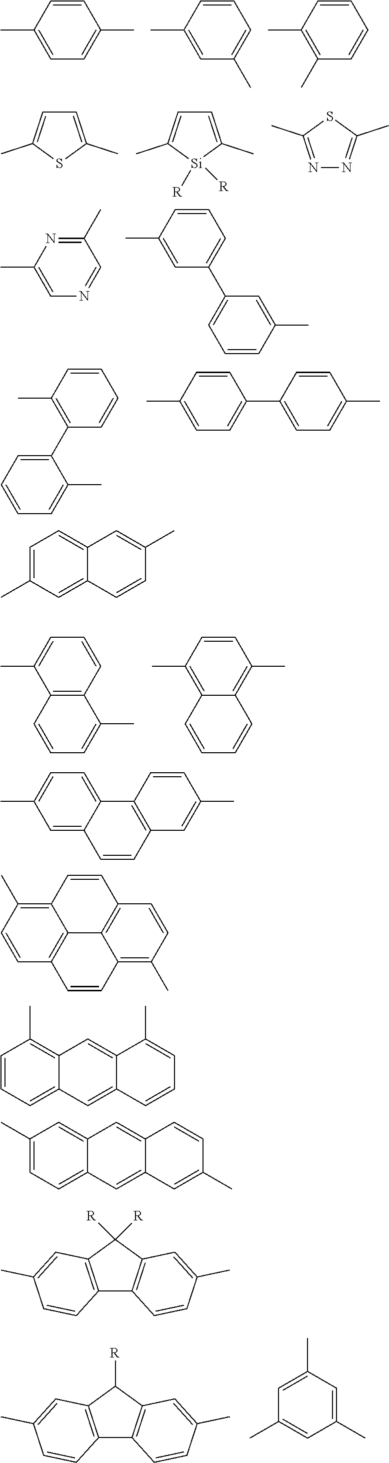

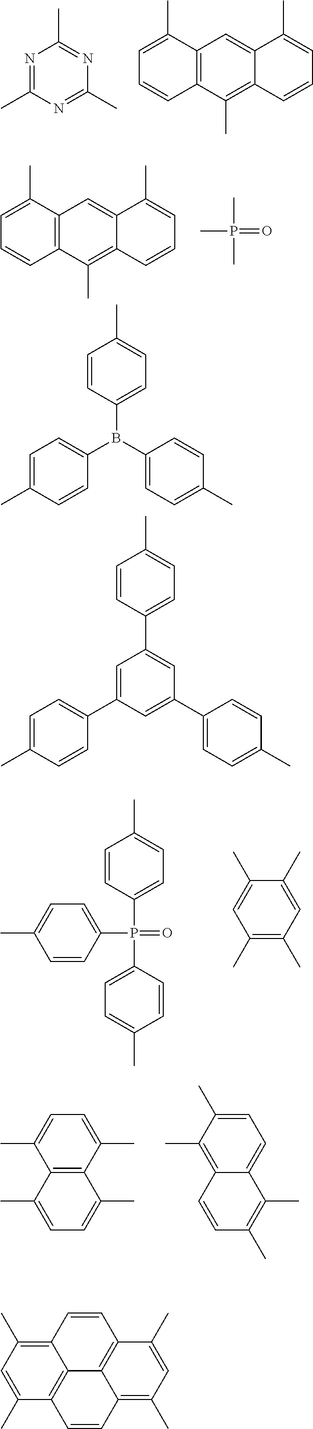

Specific examples of this phenanthroline derivative include 4,7-diphenyl-1,10-phenanthroline, 2,9-dimethyl-4,7-diphenyl-1,10-phenanthroline, 9,10-di(1,10-phenanthrolin-2-yl)anthracene, 2,6-di(1,10-phenanthrolin-5-yl)pyridine, 1,3,5-tri(1,10-phenanthrolin-5-yl)benzene, 9,9′-difluoro-bis(1,10-phenanthrolin-5-yl), bathocuproine, 1,3-bis(2-phenyl-1,10-phenanthrolin-9-yl)benzene, and the like.

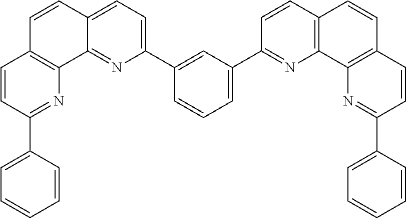 [SMILES: c1ccc(-c2ccc3ccc4ccc(-c5cccc(-c6ccc7ccc8ccc(-c9ccccc9)nc8c7n6)c5)nc4c3n2)cc1]

This phenanthroline derivative can be manufactured using known raw materials and known synthesis methods.

<Quinolinol-Based Metal Complex>

The quinolinol-based metal complex is, for example, a compound represented by the following general formula (ETM-13)

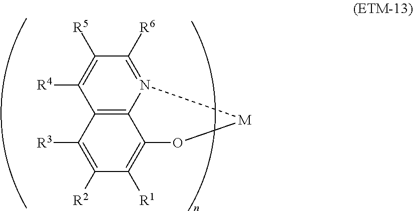

In the formula, R¹ to R⁶ each independently represent a hydrogen atom or substituents, M represents Li, Al, Ga, Be, or Zn, and n represents an integer of 1 to 3.

Specific examples of the quinolinol-based metal complex include 8-quinolinollithium, tris(8-quinolinolato)aluminum, tris(4-methyl-8-quinolinolato)aluminum, tris(5-methyl-8-quinolinolato)aluminum, tris(3,4-dimethyl-8-quiolinolato)aluminum, tris(4,5-dimethyl-8-quinolinolato)aluminum, tris(4,6-dimethyl-8-quinolinolato)aluminum, bis(2-methyl-8-quinolinolato) (phenolato)aluminum, bis(2-methyl-8-quinolinolato) (2-methylphenolato)aluminum, bis(2-methyl-8-quinolinolato) (3-methylphenolato)aluminum, bis(2-methyl-8-quinolinolato) (4-methylphenolato)aluminum, bis(2-methyl-8-quinolinolato) (2-phenylphenolato)aluminum, bis(2-methyl-8-quinolinolato) (3-phenylphenolato)aluminum, bis(2-methyl-8-quinolinolato) (4-phenylphenolato)aluminum, bis(2-methyl-8-quinolinolato) (2,3-dimethylphenolato)aluminum, bis(2-methyl-8-quinolinolato) (2,6-dimethylphenolato)aluminum, bis(2-methyl-8-quinolinolato) (3,4-dimethylphenolato)aluminum, bis(2-methyl-8-quinolinolato) (3,5-dimethylphenolato)aluminum, bis(2-methyl-8-quinolinolato) (3,5-di-t-butylphenolato)aluminum, bis(2-methyl-8-quinolinolato) (2,6-diphenylphenolato)aluminum, bis(2-methyl-8-quinolinolato) (2,4,6-triphenylphenolato)aluminum, bis(2-methyl-8-quinolinolato) (2,4,6-trimethylphenolato)aluminum, bis(2-methyl-8-quinolinolato) (2,4,5,6-tetramethylphenolato)aluminum, bis(2-methyl-8-quinolinolato) (1-naphtholato)aluminum, bis(2-methyl-8-quinolinolato) (2-naphtholato)aluminum, bis(2,4-dimethyl-8-quinolinolato) (2-phenylphenolato)aluminum, bis(2,4-dimethyl-8-quinolinolato) (3-phenylphenolato)aluminum, bis(2,4-dimethyl-8-quinolinolato) (4-phenylphenolato)aluminum, bis(2,4-dimethyl-8-quinolinolato) (3,5-dimethylphenolato)aluminum, bis(2,4-dimethyl-8-quinolinolato) (3,5-di-t-butylphenolato)aluminum, bis(2-methyl-8-quinolinolato)aluminum-μ-oxo-bis(2-methyl-8-quinolinolato)aluminum, bis(2,4-dimethyl-8-quinolinolato)aluminum-μ-oxo-bis(2,4-dimethyl-8-quinolinolato)aluminum, bis(2-methyl-4-ethyl-8-quinolinolato)aluminum-μ-oxo-bis(2-methyl-4-ethyl-8-quinolinolato)aluminum, bis(2-methyl-4-methoxy-8-quinolinolato)aluminum-μ-oxo-bis(2-methyl-4-methoxy-8-quinolinolato)aluminum, bis(2-methyl-5-cyano-8-quinolinolato)aluminum-μ-oxo-bis(2-methyl-5-cyano-8-quinolinolato)aluminum, bis(2-methyl-5-trifluoromethyl-8-quinolinolato)aluminum-μ-oxo-bis(2-methyl-5-trifluoromethyl-8-quiolinolato)aluminum, and bis(10-hydroxybenzo[h]quinoline)beryllium.

This quinolinol-based metal complex can be manufactured using known raw materials and known synthesis methods.

<Thiazole Derivative and Benzothiazole Derivative>

The thiazole derivative is, for example, a compound represented by the following formula (ETM-14-1).

ϕ-(Thiazole-based substituent)ₙ (ETM-14-1)

The benzothiazole derivative is, for example, a compound represented by the following formula (ETM-14-2).

ϕ-(Benzothiazole-based substituent)ₙ (ETM-14-2)

φ in each formula represents an n-valent aryl ring (preferably, an n-valent benzene ring, naphthalene ring, anthracene ring, fluorene ring, benzofluorene ring, phenalene ring, phenanthrene ring, or triphenylene ring), and n represents an integer of 1 to 4. A “thiazole-based substituent” or a “benzothiazole-based substituent” is a substituent in which the pyridyl group in the “pyridine-based substituent” in the formulas (ETM-2), (ETM-2-1), and (ETM-2-2) is substituted by the following thiazole group or benzothiazole group, and at least one hydrogen atom in the thiazole derivative and the benzothiazole derivative may be substituted by a deuterium atom.

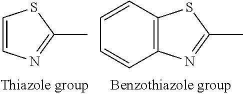

Furthermore, φ is preferably an anthracene ring or a fluorene ring. For the structure in this case, the description of the above formula (ETM-2-1) or (ETM-2-2) can be cited. For R¹¹ to R¹⁸ in each formula, the description of the above formula (ETM-2-1) or (ETM-2-2) can be cited. In the above formula (ETM-2-1) or (ETM-2-2), a form in which two pyridine-based substituents are bonded has been described. However, when these substituents are substituted by thiazole-based substituents (or benzothiazole-based substituents), both the pyridine-based substituents may be substituted by thiazole-based substituents (or benzothiazole-based substituents) (that is, n=2), or one of the pyridine-based substituents may be substituted by a thiazole-based substituent (or benzothiazole-based substituent) and the other pyridine-based substituent may be substituted by any one of R¹¹ to R¹⁸ (that is, n=1). Furthermore, for example, at least one of R¹¹ to R¹⁸ in the above formula (ETM-2-1) may be substituted by a thiazole-based substituent (or benzothiazole-based substituent) and the “pyridine-based substituent” may be substituted by any one of R¹¹ to R¹⁸.

These thiazole derivatives or benzothiazole derivatives can be manufactured using known raw materials and known synthesis methods.

The electron transport layer or the electron injection layer may further contain a substance capable of reducing a material to form the electron transport layer or the electron injection layer. As this reducing substance, various substances are used as long as having reducibility to a certain extent. For example, at least one selected from the group consisting of an alkali metal, an alkaline earth metal, a rare earth metal, an oxide of an alkali metal, a halide of an alkali metal, an oxide of an alkaline earth metal, a halide of an alkaline earth metal, an oxide of a rare earth metal, a halide of a rare earth metal, an organic complex of an alkali metal, an organic complex of an alkaline earth metal, and an organic complex of a rare earth metal, can be suitably used.

Preferable examples of the reducing substance include an alkali metal such as Na (work function 2.36 eV), K (work function 2.28 eV), Rb (work function 2.16 eV), or Cs (work function 1.95 eV); and an alkaline earth metal such as Ca (work function 2.9 eV), Sr (work function 2.0 to 2.5 eV), or Ba (work function 2.52 eV). A substance having a work function of 2.9 eV or less is particularly preferable. Among these substances, an alkali metal such as K, Rb, or Cs is a more preferable reducing substance, Rb or Cs is a still more preferable reducing substance, and Cs is the most preferable reducing substance. These alkali metals have particularly high reducing ability, and can enhance emission luminance of an organic EL element or can lengthen a lifetime thereof by adding the alkali metals in a relatively small amount to a material to form an electron transport layer or an electron injection layer. Furthermore, as the reducing substance having a work function of 2.9 eV or less, a combination of two or more kinds of these alkali metals is also preferable, and particularly, a combination including Cs, for example, a combination of Cs with Na, a combination of Cs with K, a combination of Cs with Rb, or a combination of Cs with Na and K, is preferable. By inclusion of Cs, reducing ability can be efficiently exhibited, and emission luminance of an organic EL element is enhanced or a lifetime thereof is lengthened by adding Cs to a material to form an electron transport layer or an electron injection layer.

<Negative Electrode in Organic Electroluminescent Element>

The negative electrode 108 plays a role of injecting an electron to the light emitting layer 105 through the electron injection layer 107 and the electron transport layer 106.

A material to form the negative electrode 108 is not particularly limited as long as being a substance capable of efficiently injecting an electron to an organic layer. However, a material similar to a material to form the positive electrode 102 can be used. Among these materials, a metal such as tin, indium, calcium, aluminum, silver, copper, nickel, chromium, gold, platinum, iron, zinc, lithium, sodium, potassium, cesium, or magnesium, and an alloy thereof (a magnesium-silver alloy, a magnesium-indium alloy, an aluminum-lithium alloy such as lithium fluoride/aluminum, or the like) are preferable. In order to enhance element characteristics by increasing an electron injection efficiency, lithium, sodium, potassium, cesium, calcium, magnesium, or an alloy containing these low work function-metals is effective. However, many of these low work function-metals are generally unstable in air. In order to ameliorate this problem, for example, a method using an electrode having high stability obtained by doping an organic layer with a trace amount of lithium, cesium, or magnesium is known. Other examples of a dopant that can be used include an inorganic salt such as lithium fluoride, cesium fluoride, lithium oxide, or cesium oxide. However, the dopant is not limited thereto.

Furthermore, in order to protect an electrode, a metal such as platinum, gold, silver, copper, iron, tin, aluminum, or indium, an alloy using these metals, an inorganic substance such as silica, titania, or silicon nitride, polyvinyl alcohol, vinyl chloride, a hydrocarbon-based polymer compound, or the like may be laminated as a preferable example. A method for manufacturing these electrodes is not particularly limited as long as being able to obtain conduction, such as resistance heating, electron beam deposition, sputtering, ion plating, or coating.

<Binder that May be Used in Each Layer>

The materials used in the above-described hole injection layer, hole transport layer, light emitting layer, electron transport layer, and electron injection layer can form each layer by being used singly. However, it is also possible to use the materials by dispersing the materials in a solvent-soluble resin such as polyvinyl chloride, polycarbonate, polystyrene, poly(N-vinylcarbazole), polymethyl methacrylate, polybutyl methacrylate, polyester, polysulfone, polyphenylene oxide, polybutadiene, a hydrocarbon resin, a ketone resin, a phenoxy resin, polyamide, ethyl cellulose, a vinyl acetate resin, an ABS resin, or a polyurethane resin; or a curable resin such as a phenolic resin, a xylene resin, a petroleum resin, a urea resin, a melamine resin, an unsaturated polyester resin, an alkyd resin, an epoxy resin, or a silicone resin, as a polymer binder.

<Method for Manufacturing Organic Electroluminescent Element>

Each layer constituting an organic EL element can be formed by forming thin films of the materials to constitute each layer by methods such as a vapor deposition method, resistance heating deposition, electron beam deposition, sputtering, a molecular lamination method, a printing method, a spin coating method, a casting method, and a coating method. The film thickness of each layer thus formed is not particularly limited, and can be appropriately set according to a property of a material, but is usually within a range of 2 nm to 5000 nm. The film thickness can be usually measured using a crystal oscillation type film thickness measuring apparatus or the like. In a case of forming a thin film using a vapor deposition method, vapor deposition conditions depend on the kind of a material, an intended crystal structure of a film, an association structure, and the like. It is preferable to appropriately set the vapor deposition conditions generally in ranges of a boat heating temperature of +50 to +400° C., a degree of vacuum of 10⁻⁶ to 10⁻³ Pa, a rate of vapor deposition of 0.01 to 50 nm/sec, a substrate temperature of −150 to +300° C., and a film thickness of 2 nm to 5 μm.

Next, as an example of a method for manufacturing an organic EL element, a method for manufacturing an organic EL element formed of positive electrode/hole injection layer/hole transport layer/light emitting layer including a host material and a dopant material/electron transport layer/electron injection layer/negative electrode will be described. A thin film of a positive electrode material is formed on an appropriate substrate by a vapor deposition method or the like to manufacture a positive electrode, and then thin films of a hole injection layer and a hole transport layer are formed on this positive electrode. A thin film is formed thereon by co-depositing a host material and a dopant material to obtain a light emitting layer. An electron transport layer and an electron injection layer are formed on this light emitting layer, and a thin film formed of a substance for a negative electrode is formed by a vapor deposition method or the like to obtain a negative electrode. An intended organic EL element is thereby obtained. Incidentally, in manufacturing the above organic EL element, it is also possible to manufacture the element by reversing the manufacturing order, that is, in order of a negative electrode, an electron injection layer, an electron transport layer, a light emitting layer, a hole transport layer, a hole injection layer, and a positive electrode.

In a case where a direct current voltage is applied to the organic EL element thus obtained, it is only required to apply the voltage by assuming a positive electrode as a positive polarity and assuming a negative electrode as a negative polarity. By applying a voltage of about 2 to 40 V, light emission can be observed from a transparent or semitransparent electrode side (the positive electrode or the negative electrode, or both the electrodes). This organic EL element also emits light even in a case where a pulse current or an alternating current is applied. Note that a waveform of an alternating current applied may be any waveform.

<Application Examples of Organic Electroluminescent Element>

The present invention can also be applied to a display apparatus including an organic EL element, a lighting apparatus including an organic EL element, or the like.

The display apparatus or lighting apparatus including an organic EL element can be manufactured by a known method such as connecting the organic EL element according to the present embodiment to a known driving apparatus, and can be driven by appropriately using a known driving method such as direct driving, pulse driving, or alternating driving.

Examples of the display apparatus include a panel display such as a color flat panel display; and a flexible display such as a flexible color organic electroluminescent (EL) display (see, for example, JP 10-335066 A, JP 2003-321546 A, and JP 2004-281086 A). Examples of a display method of the display include a matrix method and/or a segment method. Note that the matrix display and the segment display may co-exist in the same panel.

In the matrix, pixels for display are arranged two-dimensionally as in a lattice form or a mosaic form, and characters or images are displayed by an assembly of pixels. The shape or size of a pixel depends on intended use. For example, for display of images and characters of a personal computer, a monitor, or a television, square pixels each having a size of 300 μm or less on each side are usually used, and in a case of a large-sized display such as a display panel, pixels having a size in the order of millimeters on each side are used. In a case of monochromic display, it is only required to arrange pixels of the same color. However, in a case of color display, display is performed by arranging pixels of red, green, and blue. In this case, typically, delta type display and stripe type display are available. For this matrix driving method, either a line sequential driving method or an active matrix method may be employed. The line sequential driving method has an advantage of having a simpler structure. However, in consideration of operation characteristics, the active matrix method may be superior. Therefore, it is necessary to use the line sequential driving method and the active matrix method properly according to intended use.

In the segment method (type), a pattern is formed so as to display predetermined information, and a determined region emits light. Examples of the segment method include display of time or temperature in a digital clock or a digital thermometer, display of a state of operation in an audio instrument or an electromagnetic cooker, and panel display in an automobile.

Examples of the lighting apparatus include a lighting apparatuses for indoor lighting or the like, and a backlight of a liquid crystal display apparatus (see, for example, JP 2003-257621 A, JP 2003-277741 A, and JP 2004-119211 A). The backlight is mainly used for enhancing visibility of a display apparatus that is not self-luminous, and is used in a liquid crystal display apparatus, a timepiece, an audio apparatus, an automotive panel, a display plate, a sign, and the like.

Particularly, in a backlight for use in a liquid crystal display apparatus, among the liquid crystal display apparatuses, for use in a personal computer in which thickness reduction has been a problem to be solved, in consideration of difficulty in thickness reduction because a conventional type backlight is formed from a fluorescent lamp or a light guide plate, a backlight using the luminescent element according to the present embodiment is characterized by its thinness and lightweightness.

### EXAMPLES

Hereinafter, the present invention will be described more specifically by way of Examples, but the present invention is not limited thereto. First, synthesis examples of a compound represented by the formula (1) and a compound represented by the formula (2) will be described below.

### Synthesis Example (1)

### Synthesis of Compound (1-1152): 9-([1,1′-biphenyl]-4-yl)-5,12-diphenyl-5,9-dihydro-5,9-diaza-13b-boranaphtho[3,2,1-de]anthracene

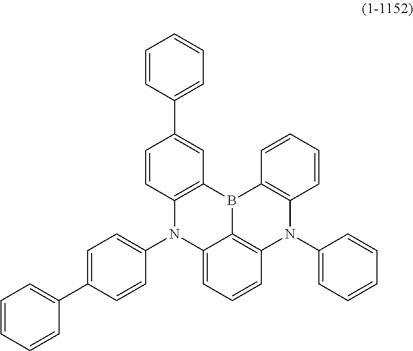 [SMILES: c1ccc(-c2ccc(N3c4ccc(-c5ccccc5)cc4B4c5ccccc5N(c5ccccc5)c5cccc3c54)cc2)cc1]

In a nitrogen atmosphere, a flask containing diphenylamine (37.5 g), 1-bromo-2,3-dichlorobenzene (50.0 g), Pd-132 (Johnson Matthey) (0.8 g), NaOtBu (32.0 g) and xylene (500 ml) was heated and stirred for 4 hours at 80° C., subsequently the temperature of the mixture was increased to 120° C., and the mixture was heated and stirred for three hours. The reaction liquid was cooled to room temperature, subsequently water and ethyl acetate were added thereto, and the mixture was partitioned. Subsequently, purification was performed by silica gel column chromatography (eluent: toluene/heptane=1/20 (volume ratio)), and thus 2,3-dichloro-N,N-diphenylaniline (63.0 g) was obtained.

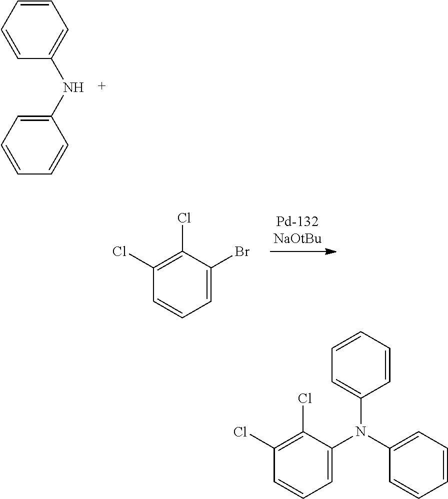 [SMILES: c1ccc(Nc2ccccc2)cc1 ; Clc1cccc(Br)c1Cl ; Clc1cccc(N(c2ccccc2)c2ccccc2)c1Cl]

In a nitrogen atmosphere, a flask containing 2,3-dichloro-N,N-diphenylaniline (16.2 g), di([1,1′-biphenyl]-4-yl)amine (15.0 g), Pd-132 (Johnson Matthey) (0.3 g), NaOtBu (6.7 g) and xylene (150 ml) was heated and stirred for one hour at 120° C. The reaction liquid was cooled to room temperature, subsequently water and ethyl acetate were added thereto, and the mixture was partitioned. Subsequently, purification was performed using a silica gel short pass column (eluent: heated toluene) and was further washed with a mixed solvent (heptane/ethyl acetate=1 (volume ratio)). Thus, N,N′-di([1,1′-bipheyl]-4-yl)-2-chloro-N³,N³-diphenylbenzene-1,3-diamine (22.0 g) was obtained.

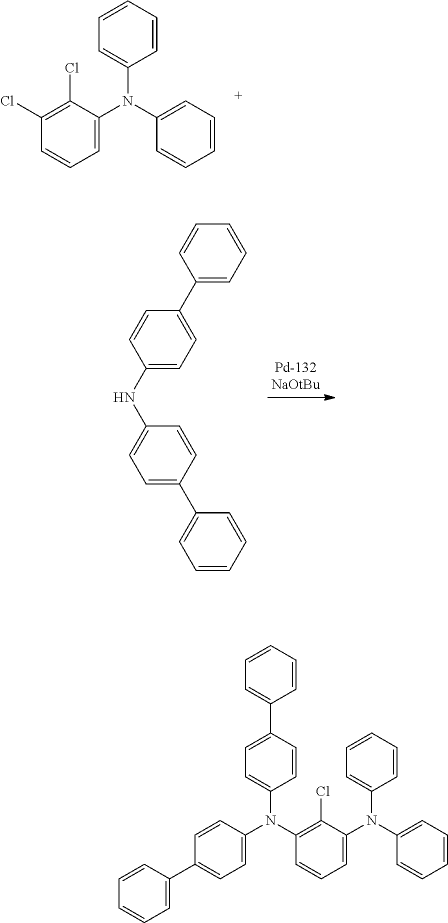 [SMILES: Clc1cccc(N(c2ccccc2)c2ccccc2)c1Cl ; c1ccc(-c2ccc(Nc3ccc(-c4ccccc4)cc3)cc2)cc1 ; Clc1c(N(c2ccccc2)c2ccccc2)cccc1N(c1ccc(-c2ccccc2)cc1)c1ccc(-c2ccccc2)cc1]

A 1.6 M tert-butyllithium pentane solution (37.5 ml) was put into a flask containing N′,N¹-di([1,1′-biphenyl]-4-yl)-2-chloro-N³,N³-diphenylbenzene-1,3-diamine (22.0 g) and tert-butylbenzene (130 ml) at −30° C. in a nitrogen atmosphere. After completion of dropwise addition, the temperature of the mixture was increased to 60° C., the mixture was stirred for one hour, and then components having boiling points lower than that of tert-butylbenzene were distilled off under reduced pressure. The residue was cooled to −30° C., boron tribromide (6.2 ml) was added thereto, the temperature of the mixture was raised to room temperature, and the mixture was stirred for 0.5 hours. Thereafter, the mixture was cooled again to 0° C., N,N-diisopropylethylamine (12.8 ml) was added thereto, and the mixture was stirred at room temperature until heat generation was settled. Subsequently, the temperature of the mixture was raised to 120° C., and the mixture was heated and stirred for two hours. The reaction liquid was cooled to room temperature, an aqueous solution of sodium acetate that had been cooled in an ice bath and then ethyl acetate were added thereto, and the mixture was partitioned. Subsequently, purification was performed using a silica gel short pass column (eluent: heated chlorobenzene). The purification product was washed with refluxed heptane and refluxed ethyl acetate, and then was reprecipitated from chlorobenzene. Thus, a compound (5.1 g) represented by formula (1-1152) was obtained.

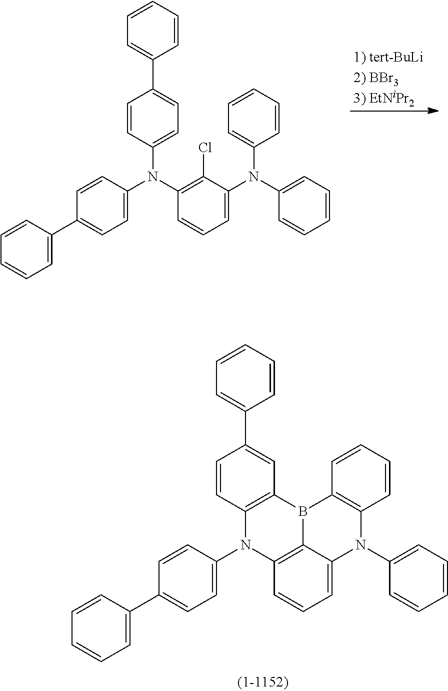 [SMILES: Clc1c(N(c2ccccc2)c2ccccc2)cccc1N(c1ccc(-c2ccccc2)cc1)c1ccc(-c2ccccc2)cc1 ; c1ccc(-c2ccc(N3c4ccc(-c5ccccc5)cc4B4c5ccccc5N(c5ccccc5)c5cccc3c54)cc2)cc1]

The structure of the compound thus obtained was identified by an NMR analysis.

¹H-NMR (400 MHz, CDCl₃): δ=9.17 (s, 1H), 8.99 (d, 1H), 7.95 (d, 2H), 7.68-7.78 (m, 7H), 7.60 (t, 1H), 7.40-7.56 (m, 10H), 7.36 (t, 1H), 7.30 (m, 2H), 6.95 (d, 1H), 6.79 (d, 1H), 6.27 (d, 1H), 6.18 (d, 1H).

### Synthesis Example (2)

### Synthesis of Compound (1-2679): 9-([1,1′-biphenyl]-4-yl)-N,N,5,12-tetraphenyl-5,9-dihydro-5,9-diaza-13b-boranaphtho[3,2,1-de]anthracene-3-amine

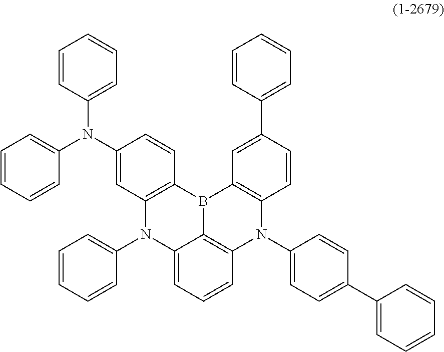 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1]

In a nitrogen atmosphere, a flask containing N¹,N¹,N³-triphenylbenzene-1,3-diamine (51.7 g), 1-bromo-2,3-dichlorobenzene (35.0 g), Pd-132 (0.6 g), NaOtBu (22.4 g), and xylene (350 ml) was heated and stirred for two hours at 90° C. The reaction liquid was cooled to room temperature, subsequently water and ethyl acetate were added thereto, and the mixture was partitioned. Subsequently, purification was performed by silica gel column chromatography (eluent: toluene/heptane=5/5 (volume ratio)), and thus N¹-(2,3-dichlorophenyl)-N¹,N³,N³-triphenylbenzene-1,3-diamine (61.8 g) was obtained.

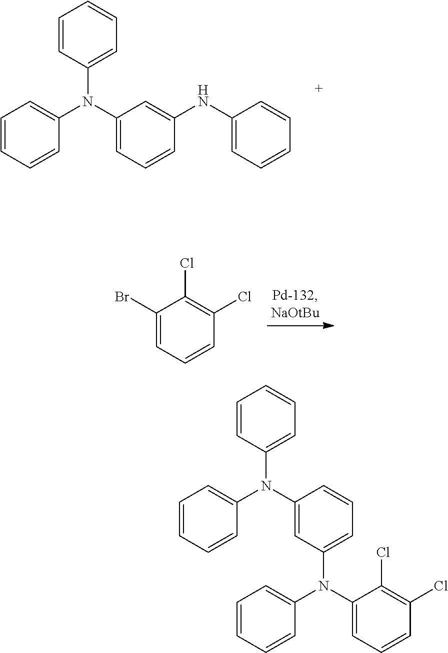 [SMILES: c1ccc(Nc2cccc(N(c3ccccc3)c3ccccc3)c2)cc1 ; Clc1cccc(Br)c1Cl ; Clc1cccc(N(c2ccccc2)c2cccc(N(c3ccccc3)c3ccccc3)c2)c1Cl]

In a nitrogen atmosphere, a flask containing N¹-(2,3-dichlorophenyl)-N¹,N³,N³-triphenylbenzene-1,3-diamine (15.0 g), di([1,1′-biphenyl]-4-yl)amine (10.0 g), Pd-132 (0.2 g), NaOtBu (4.5 g), and xylene (70 ml) was heated and stirred for one hour at 120° C. The reaction liquid was cooled to room temperature, subsequently water and toluene were added thereto, and the mixture was partitioned. Subsequently, purification was performed using a silica gel short pass column (eluent: toluene). An oily material thus obtained was reprecipitated with an ethyl acetate/heptane mixed solvent, and thus N,N-di([1,1′-biphenyl]-4-yl)-2-chloro-N³-(3-(diphenylamino)phenyl)-N³-phenylbenzene-1,3-diamine (18.5 q) was obtained.

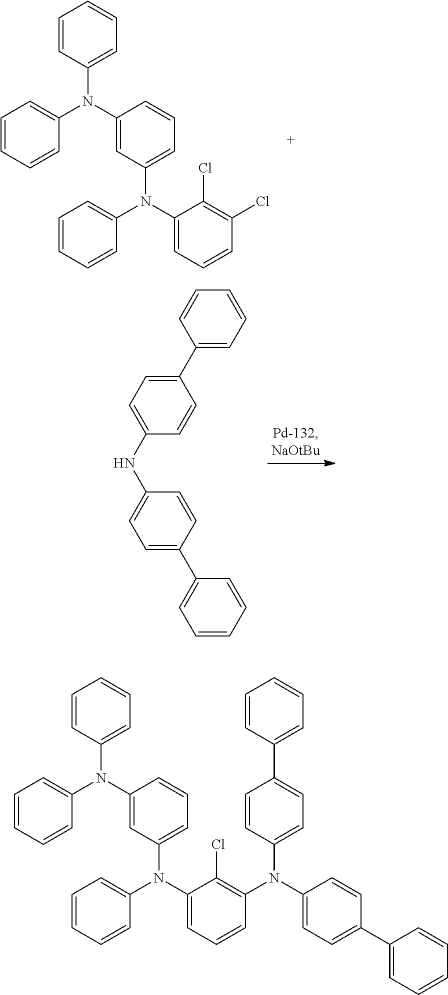

A 1.7 M t-butyllithium pentane solution (27.6 ml) was put into a flask containing N′,N′-di([1,1′-biphenyl]-4-yl)-2-chloro-N³-(3-(diphenylamino)phenyl)-N³-phenylbenzene-1,3-diamine (18.0 g) and t-butylbenzene (130 ml) in a nitrogen atmosphere, while the flask was cooled in an ice bath. After completion of dropwise addition, the temperature was increased to 60° C., the mixture was stirred for three hours, and then components having boiling points that were lower than that of t-butylbenzene were distilled off under reduced pressure. The residue was cooled to −50° C., boron tribromide (4.5 ml) was added thereto, the temperature of the mixture was raised to room temperature, and the mixture was stirred for 0.5 hours. Thereafter, the mixture was cooled again in an ice bath, and N,N-diisopropylethylamine (8.2 ml) was added thereto. The mixture was stirred at room temperature until heat generation was settled, subsequently the temperature of the mixture was raised to 120° C., and the mixture was heated and stirred for one hour. The reaction liquid was cooled to room temperature, an aqueous solution of sodium acetate that had been cooled in an ice bath and then ethyl acetate were added thereto, and the mixture was partitioned. Subsequently, dissolution in hot chlorobenzene was performed, and purification was performed using a silica gel short pass column (eluent: hot toluene). The purification product was further recrystallized from chlorobenzene, and thus a compound (3.0 g) represented by formula (1-2679) was obtained.

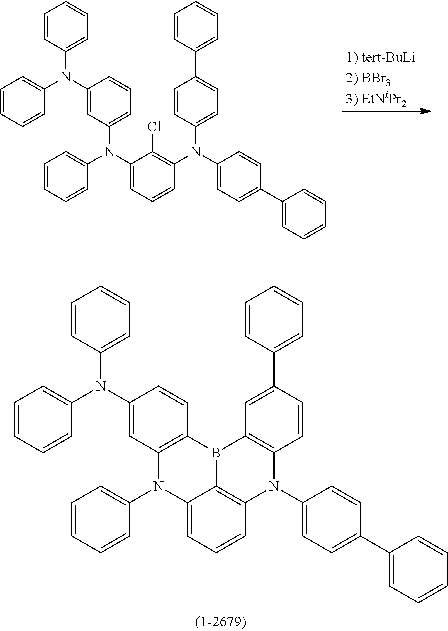 [SMILES: Clc1c(N(c2ccc(-c3ccccc3)cc2)c2ccc(-c3ccccc3)cc2)cccc1N(c1ccccc1)c1cccc(N(c2ccccc2)c2ccccc2)c1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1]

The structure of the compound thus obtained was identified by an NMR analysis.

¹H-NMR (400 MHz, CDCl₃): δ=9.09 (m, 1H), 8.79 (d, 1H), 7.93 (d, 2H), 7.75 (d, 2H), 7.72 (d, 2H), 7.67 (m, 1H), 7.52 (t, 2H), 7.40-7.50 (m, 7H), 7.27-7.38 (m, 2H), 7.19-7.26 (m, 7H), 7.11 (m, 4H), 7.03 (t, 2H), 6.96 (dd, 1H), 6.90 (d, 1H), 6.21 (m, 2H), 6.12 (d, 1H)

### Synthesis Example (3)

### Synthesis of Compound (1-2676): 9-([1,1′-biphenyl]-3-yl)-N,N,5,11-tetraphenyl-5,9-dihydro-5,9-diaza-13b-boranaphtho[3,2,1-de]anthracene-3-amine

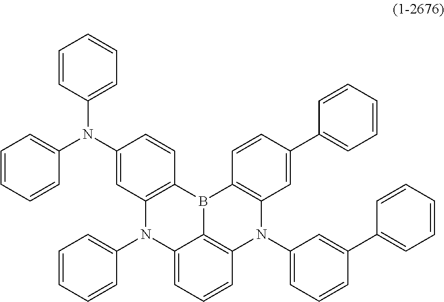 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1]

In a nitrogen atmosphere, a flask containing [1,1′-biphenyl]-3-amine (19.0 g), 4-bromo-1,1′-biphenyl (25.0 g), Pd-132 (0.8 g), NaOtBu (15.5 g) and xylene (200 ml) was heated and stirred for six hours at 120° C. The reaction liquid was cooled to room temperature, subsequently water and ethyl acetate were added thereto, and the mixture was partitioned. Subsequently, purification was performed by silica gel column chromatography (eluent: toluene/heptane=5/5 (volume ratio)). A solid obtained by distilling off the solvent under reduced pressure was washed with heptane, and thus di([1,1′-biphenyl]-3-yl)amine (30.0 g) was obtained.

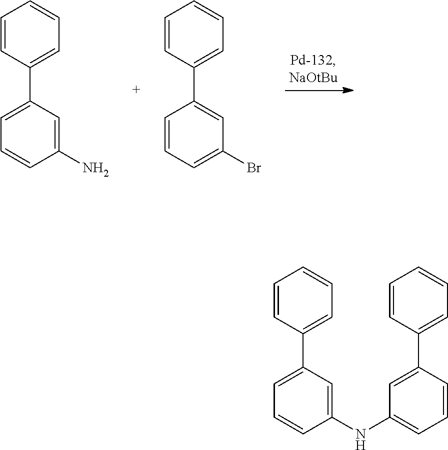 [SMILES: Nc1cccc(-c2ccccc2)c1 ; Brc1cccc(-c2ccccc2)c1 ; c1ccc(-c2cccc(Nc3cccc(-c4ccccc4)c3)c2)cc1]

In a nitrogen atmosphere, a flask containing N¹-(2,3-dichlorophenyl)-N¹,N³,N³-triphenylbenzene-1,3-diamine (15.0 g), di([1,1′-biphenyl]-3-yl)amine (10.0 g), Pd-132 (0.2 g), NaOtBu (4.5 g), and xylene (70 ml) was heated and stirred for one hour at 120° C. The reaction liquid was cooled to room temperature, subsequently water and ethyl acetate were added thereto, and the mixture was partitioned. Subsequently, purification was performed by silica gel column chromatography (eluent: toluene/heptane=5/5 (volume ratio)). A fraction containing a desired product was reprecipitated by distilling off the solvent under reduced pressure, and thus N′,N-di([1,1′-biphenyl]-3-yl)-2-chloro-N³-(3-(diphenylamino)phenyl)-N³-phenylbenzene-1,3-diamine (20.3 g) was obtained.

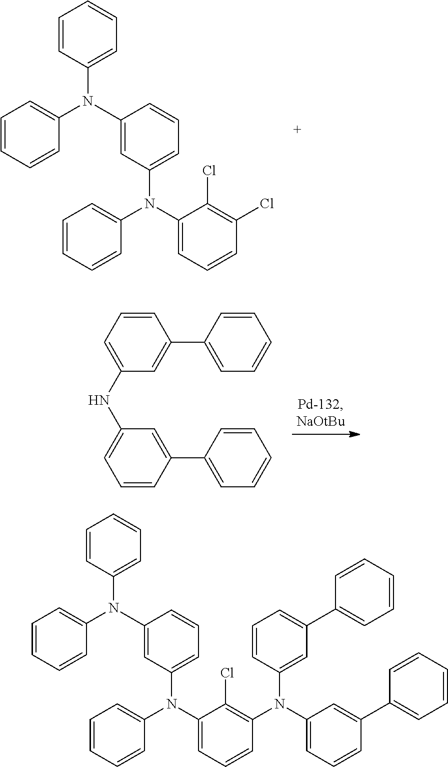

A 1.6 M t-butyllithium pentane solution (32.6 ml) was put into a flask containing N,N′-di([1,1′-biphenyl]-3-yl)-2-chloro-N³-(3-(diphenylamino)phenyl)-N³-phenylbenzene-1,3-diamine (20.0 g) and t-butylbenzene (150 ml) in a nitrogen atmosphere, while the flask was cooled in an ice bath. After completion of dropwise addition, the temperature was increased to 60° C., the mixture was stirred for two hours, and then the components having boiling points that were lower than that of t-butylbenzene were distilled off under reduced pressure. The residue was cooled to −50° C., boron tribromide (5.0 ml) was added thereto, the temperature of the mixture was raised to room temperature, and the mixture was stirred for 0.5 hours. Thereafter, the mixture was cooled again in an ice bath, and N,N-diisopropylethylamine (9.0 ml) was added thereto. The mixture was stirred at room temperature until heat generation was settled, subsequently the temperature was raised to 120° C., and the mixture was heated and stirred for 1.5 hours. The reaction liquid was cooled to room temperature, an aqueous solution of sodium acetate that had been cooled in an ice bath and then ethyl acetate were added thereto, and the mixture was partitioned. Subsequently, purification was performed by silica gel column chromatography (eluent: toluene/heptane=5/5). Furthermore, the purification product was reprecipitated using a toluene/heptane mixed solvent and a chlorobenzene/ethyl acetate mixed solvent, and thus a compound (5.0 g) represented by formula (1-2676) was obtained.

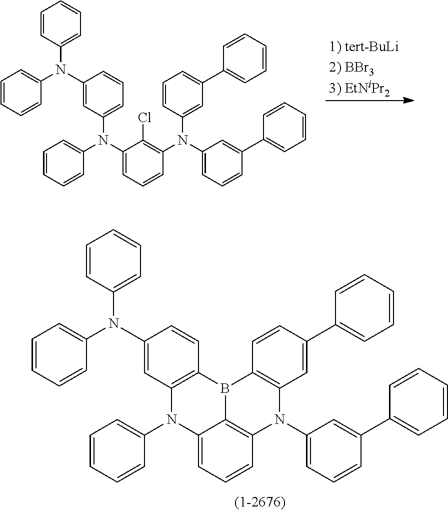 [SMILES: Clc1c(N(c2cccc(-c3ccccc3)c2)c2cccc(-c3ccccc3)c2)cccc1N(c1ccccc1)c1cccc(N(c2ccccc2)c2ccccc2)c1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

The structure of the compound thus obtained was identified by an NMR analysis.

¹H-NMR (400 MHz, CDCl₃): δ=8.93 (d, 1H), 8.77 (d, 1H), 7.84 (m, 1H), 7.77 (t, 1H), 7.68 (m, 3H), 7.33-7.50 (m, 12H), 7.30 (t, 1H), 7.22 (m, 7H), 7.11 (m, 4H), 7.03 (m, 3H), 6.97 (dd, 1H), 6.20 (m, 2H), 6.11 (d, 1H)).

### Synthesis Example (4)

### Synthesis of Compound (1-2680): N³,N³,N¹¹,N¹¹,5,9-hexaphenyl-5,9-diaza-13b-boranaphtho[3,2,1-de]anthracene-3,11-diamine

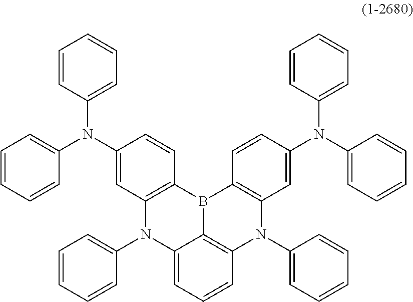 [SMILES: c1ccc(N(c2ccccc2)c2ccc3c(c2)N(c2ccccc2)c2cccc4c2B3c2ccc(N(c3ccccc3)c3ccccc3)cc2N4c2ccccc2)cc1]

In a nitrogen atmosphere, a flask containing 3-nitroaniline (25.0 g), iodobenzene (81.0 g), copper iodide (3.5 g), potassium carbonate (100.0 g) and ortho-dichlorobenzene (250 ml) was heated and stirred for 14 hours at a reflux temperature. The reaction liquid was cooled to room temperature, subsequently aqueous ammonia was added thereto, and the mixture was partitioned. Subsequently, purification was performed by silica gel column chromatography (eluent: toluene/heptane=3/7 (volume ratio)), and thus 3-nitro-N,N-diphenylaniline (44.0 g) was obtained.

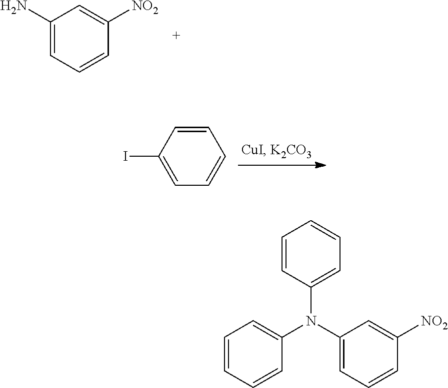 [SMILES: Nc1cccc([N+](=O)[O-])c1 ; Ic1ccccc1 ; O=[N+]([O-])c1cccc(N(c2ccccc2)c2ccccc2)c1]

In a nitrogen atmosphere, a flask containing acetic acid (440 mL) had been cooled in an ice bath, and zinc (50.0 g) was added and stirred. 3-Nitro-N,N-diphenylaniline (44.0 g) was added to this solution in divided portions such that the reaction temperature would not noticeably increase. After completion of the addition, the mixture was stirred for 30 minutes at room temperature, and any loss of the raw material was checked. After completion of the reaction, a supernatant was collected by decantation and was neutralized with sodium carbonate, and the resultant was extracted with ethyl acetate. Subsequently, the resultant was purified by silica gel column chromatography (eluent: toluene/heptane=9/1 (volume ratio)). A fraction containing an intended product was reprecipitated by distilling off the solvent under reduced pressure and adding heptane thereto. Thus, N′,N¹-diphenylbenzene-1,3-diamine (36.0 g) was obtained.

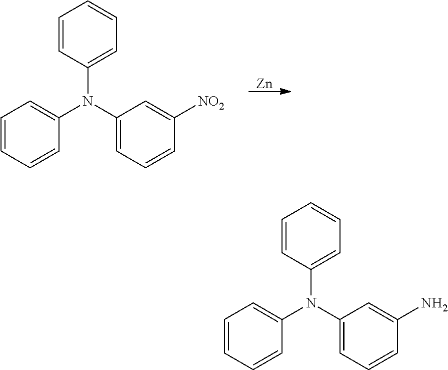

In a nitrogen atmosphere, a flask containing N′,N¹-diphenylbenzene-1,3-diamine (60.0 g), Pd-132 (1.3 g), NaOtBu (33.5 g) and xylene (300 ml) was heated and stirred at 120° C. To this solution, a xylene (50 ml) solution of bromobenzene (36.2 g) was slowly added dropwise, and after completion of the dropwise addition, the mixture was heated and stirred for one hour. The reaction liquid was cooled to room temperature, subsequently water and ethyl acetate were added thereto, and the mixture was partitioned. Subsequently, purification was performed by silica gel column chromatography (eluent: toluene/heptane=5/5 (volume ratio)), and thus N¹,N¹,N³-triphenylbenzene-1,3-diamine (73.0 g) was obtained.

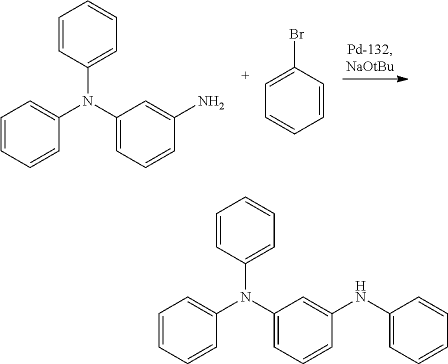 [SMILES: Nc1cccc(N(c2ccccc2)c2ccccc2)c1 ; Brc1ccccc1 ; c1ccc(Nc2cccc(N(c3ccccc3)c3ccccc3)c2)cc1]

In a nitrogen atmosphere, a flask containing N¹,N¹,N³-triphenylbenzene-1,3-diamine (20.0 g), 1-bromo-2,3-dichlorobenzene (6.4 g), Pd-132 (0.2 g), NaOtBu (6.8 g), and xylene (70 ml) was heated and stirred for two hours at 120° C. The reaction liquid was cooled to room temperature, subsequently water and ethyl acetate were added thereto, and the mixture was partitioned. Subsequently, purification was performed by silica gel column chromatography (eluent: toluene/heptane=4/6 (volume ratio)), and thus N¹,N¹′-(2-chloro-1,3-phenylene)bis (N¹,N³,N³-triphenylbenzene-1,3-diamine) (15.0 g) was obtained.

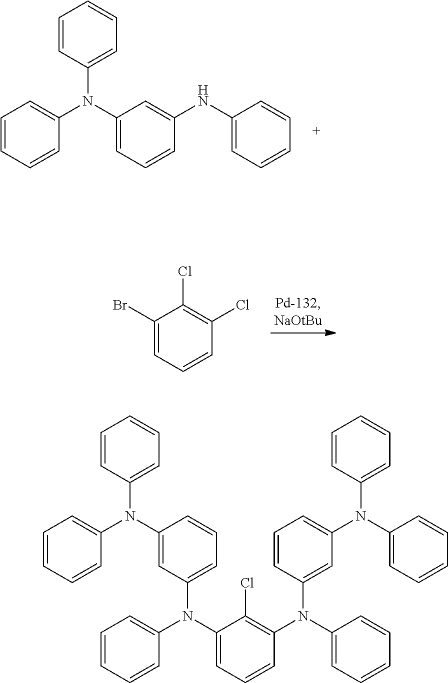 [SMILES: c1ccc(Nc2cccc(N(c3ccccc3)c3ccccc3)c2)cc1 ; Clc1cccc(Br)c1Cl ; Clc1c(N(c2ccccc2)c2cccc(N(c3ccccc3)c3ccccc3)c2)cccc1N(c1ccccc1)c1cccc(N(c2ccccc2)c2ccccc2)c1]

A 1.7 M t-butyllithium pentane solution (18.1 ml) was introduced into a flask containing N¹,N¹′-(2-chloro-1,3-phenylene)bis(N¹,N³,N³-triphenylbenzene-1,3-diamine) (12.0 g) and t-butylbenzene (100 ml) in a nitrogen atmosphere, while the flask was cooled in an ice bath. After completion of dropwise addition, the temperature was increased to 60° C., the mixture was stirred for two hours, and then the components having boiling points that were lower than that of t-butylbenzene were distilled off under reduced pressure. The residue was cooled to −50° C., boron tribromide (2.9 ml) was added thereto, the temperature of the mixture was increased to room temperature, and the mixture was stirred for 0.5 hours. Thereafter, the mixture was cooled again in an ice bath, and N,N-diisopropylethylamine (5.4 ml) was added thereto. The mixture was stirred at room temperature until heat generation was settled, subsequently the temperature of the mixture was increased to 120° C., and the mixture was heated and stirred for three hours. The reaction liquid was cooled to room temperature, and an aqueous solution of sodium acetate that had been cooled in an ice bath and then ethyl acetate were added to the reaction liquid. An insoluble solid was separated by filtration, and then the liquid was partitioned. Subsequently, purification was performed by silica gel column chromatography (eluent: toluene/heptane=5/5 (volume ratio)). The purification product was further washed with hot heptane and ethyl acetate, and then was reprecipitated with a toluene/ethyl acetate mixed solvent. Thus, a compound (2.0 g) represented by formula (1-2680) was obtained.

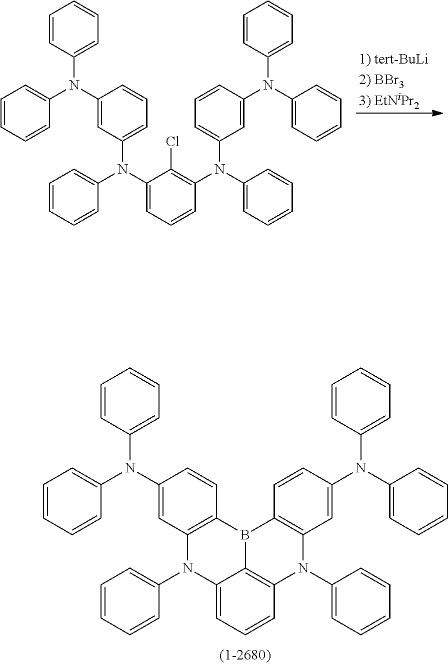 [SMILES: Clc1c(N(c2ccccc2)c2cccc(N(c3ccccc3)c3ccccc3)c2)cccc1N(c1ccccc1)c1cccc(N(c2ccccc2)c2ccccc2)c1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

The structure of the compound thus obtained was identified by an NMR analysis.

¹H-NMR (400 MHz, CDCl₃): δ=8.65 (d, 2H), 7.44 (t, 4H), 7.33 (t, 2H), 7.20 (m, 12H), 7.13 (t, 1H), 7.08 (m, 8H), 7.00 (t, 4H), 6.89 (dd, 2H), 6.16 (m, 2H), 6.03 (d, 2H).

### Synthesis Example (5)

### Synthesis of Compound (1-2621): 2,12-di-t-butyl-5,9-bis(4-(t-butyl)phenyl)-5,9-dihydro-5,9-diaza-13b-boranaphtho[3,2,1-de]anthracene

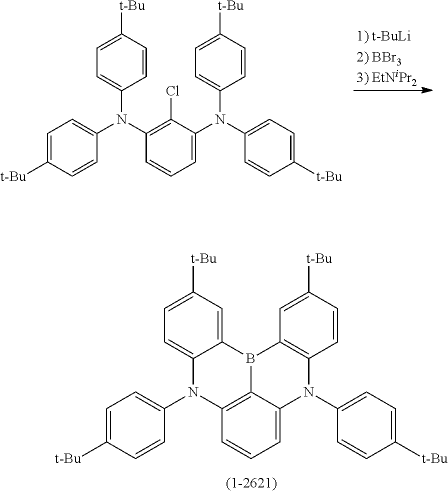 [SMILES: CC(C)(C)c1ccc(N(c2ccc(C(C)(C)C)cc2)c2cccc(N(c3ccc(C(C)(C)C)cc3)c3ccc(C(C)(C)C)cc3)c2Cl)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1]

The compound represented by formula (1-2621) was synthesized using a similar method to that in the Synthesis Example described above.

The structure of the compound thus obtained was identified by NMR measurement.

¹H-NMR (500 MHz, CDCl₃): δ=1.46 (s, 18H), 1.47 (s, 18H), 6.14 (d, 2H), 6.75 (d, 2H), 7.24 (t, 1H), 7.29 (d, 4H), 7.52 (dd, 2H), 7.67 (d, 4H), 8.99 (d, 2H).

### Synthesis Example (6)

### Synthesis of Compound (1-2619): 2,12-di-t-butyl-5,9-bis(4-(t-butyl)phenyl)-7-methyl-5,9-dihydro-5,9-diaza-13b-boranaphtho[3,2,1-de]anthracene

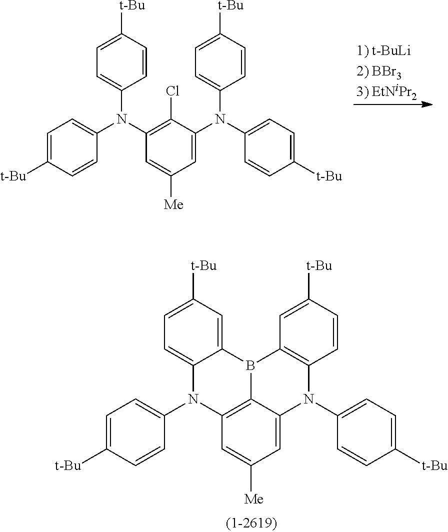 [SMILES: Cc1cc(N(c2ccc(C(C)(C)C)cc2)c2ccc(C(C)(C)C)cc2)c(Cl)c(N(c2ccc(C(C)(C)C)cc2)c2ccc(C(C)(C)C)cc2)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

The compound represented by formula (1-2619) was synthesized using a similar method to that in the Synthesis Example described above.

The structure of the compound thus obtained was identified by NMR measurement. ¹H-NMR (500 MHz, CDCl₃): δ=1.47 (s, 36H), 2.17 (s, 3H), 5.97 (s, 2H), 6.68 (d, 2H), 7.28 (d, 4H), 7.49 (dd, 2H), 7.67 (d, 4H), 8.97 (d, 2H).

### Synthesis Example (7)

### Synthesis of Compound (1-5101): 15,15-dimethyl-N,N-diphenyl-15H-5,9-dioxa-16b-boraindeno[1,2-b]naphtho[1,2,3-fg]anthracen-13-amine

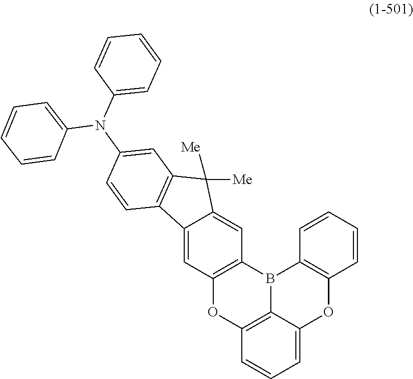 [SMILES: CC1(C)c2cc(N(c3ccccc3)c3ccccc3)ccc2-c2cc3c(cc21)B1c2ccccc2Oc2cccc(c21)O3]

In a nitrogen atmosphere, a flask containing methyl 4-methoxysalicylate (50.0 g) and pyridine (dehydrated) (350 ml) was cooled in an ice bath. Subsequently, trifluoromethanesulfonic anhydride (154.9 g) was dropwise added to this solution. After completion of the dropwise addition, the ice bath was removed, the solution was stirred at room temperature for two hours, and water was added thereto to stop the reaction. Toluene was added thereto, and the solution was partitioned. Thereafter, purification by silica gel short pass column chromatography (eluent: toluene) was performed to obtain methyl 4-methoxy-2-(((trifluoromethyl) sulfonyl) oxy) benzoate (86.0 g).

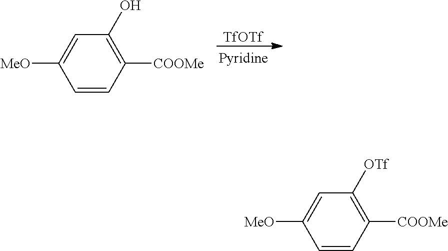 [SMILES: "TfOTf" ; COC(=O)c1ccc(OC)cc1O ; COC(=O)c1ccc(OC)cc1OS(=O)(=O)C(F)(F)F]

In a nitrogen atmosphere, Pd(PPh₃)₄ (2.5 g) was added to a suspension solution of methyl 4-methoxy-2-(((trifluoromethyl) sulfonyl) oxy) benzoate (23.0 g), (4-(diphenylamino) phenyl) boronic acid (25.4 g), tripotassium phosphate (31.1 g), toluene (184 ml), ethanol (27.6 ml), and water (27.6 ml), and the resulting mixture was stirred at a reflux temperature for three hours. The reaction liquid was cooled to room temperature, water and toluene were added thereto, and the solution was partitioned. A solvent of an organic layer was distilled off under reduced pressure. The obtained solid was purified by silica gel column chromatography (eluent: mixed solvent of heptane/toluene) to obtain methyl 4′-(diphenylamino)-5-methoxy-[1,1′-biphenyl]-2-carboxylate (29.7 g). In this case, referring to the method described on page 94 of “Guide to Organic Chemistry Experiment (1)-Substance Handling Method and Separation and Purification Method”, Kagaku-Dojin Publishing Company, INC., the proportion of toluene in a developing liquid was gradually increased, and an intended product was thereby eluted.

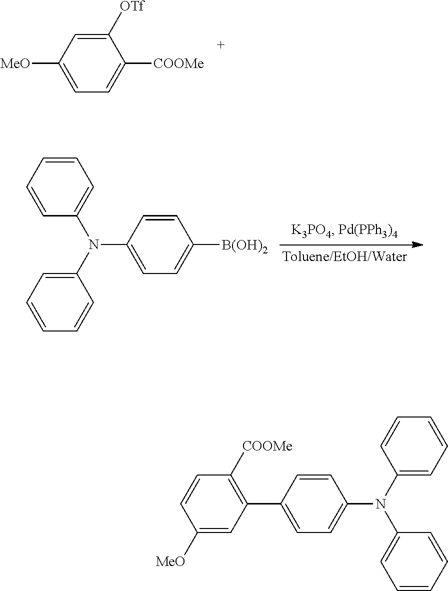 [SMILES: COC(=O)c1ccc(OC)cc1OS(=O)(=O)C(F)(F)F ; OB(O)c1ccc(N(c2ccccc2)c2ccccc2)cc1 ; COC(=O)c1ccc(OC)cc1-c1ccc(N(c2ccccc2)c2ccccc2)cc1]

In a nitrogen atmosphere, a THF (111.4 ml) solution having methyl 4′-(diphenylamino)-5-methoxy-[1,1′-biphenyl]-2-carboxylate (11.4 g) dissolved therein was cooled in a water bath. To the solution, a methyl magnesium bromide THF solution (1.0 M, 295 ml) was dropwise added. After completion of the dropwise addition, the water bath was removed, and the solution was heated to a reflux temperature, and stirred for four hours. Thereafter, the solution was cooled in an ice bath, an ammonium chloride aqueous solution was added thereto to stop the reaction, ethyl acetate was added thereto, and the solution was partitioned. Thereafter, a solvent was distilled off under reduced pressure. The obtained solid was purified by silica gel column chromatography (eluent: toluene) to obtain 2-(5′-(diphenylamino)-5-methoxy-[1,1′-biphenyl]-2-yl) propan-2-ol (8.3 g).

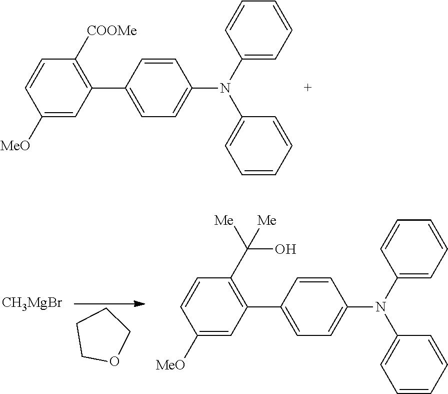 [SMILES: COC(=O)c1ccc(OC)cc1-c1ccc(N(c2ccccc2)c2ccccc2)cc1 ; "CH3MgBr" ; C1CCOC1 ; COc1ccc(C(C)(C)O)c(-c2ccc(N(c3ccccc3)c3ccccc3)cc2)c1]

In a nitrogen atmosphere, a flask containing 2-(5′-(diphenylamino)-5-methoxy-[1,1′-biphenyl]-2-yl) propan-2-ol (27.0 g), a solid acid catalyst (TAYCACURE-15 manufactured by TAYCA, acid value: 35 mg KOH/g, specific surface area: 260 m²/g, average pore diameter: 15 nm) (13.5 g), and toluene (162 ml) was stirred at a reflux temperature for two hours. The reaction liquid was cooled to room temperature and caused to pass through a silica gel short pass column (eluent: toluene) to remove TAYCACURE-15. Thereafter, a solvent was distilled off under reduced pressure to obtain 6-methoxy-9,9′-dimethyl-N,N-diphenyl-9H-fluoren-2-amine (25.8 g).

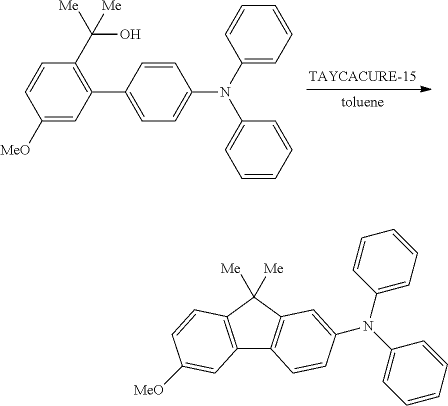 [SMILES: COc1ccc(C(C)(C)O)c(-c2ccc(N(c3ccccc3)c3ccccc3)cc2)c1 ; COc1ccc2c(c1)-c1ccc(N(c3ccccc3)c3ccccc3)cc1C2(C)C]

In a nitrogen atmosphere, a flask containing 6-methoxy-9,9′-dimethyl-N,N-diphenyl-9H-fluoren-2-amine (25.0 g), pyridine hydrochloride (36.9 g), and N-methyl-2-pyrrolidone (NMP) (22.5 ml) was stirred at a reflux temperature for six hours. The reaction liquid was cooled to room temperature, water and ethyl acetate were added thereto, and the resulting solution was partitioned. The solvent was distilled off under reduced pressure. Thereafter, the residue was purified by silica gel column chromatography (eluent: toluene) to obtain 7-(diphenylamino)-9,9′-dimethyl-9H-fluoren-3-ol (22.0 g).

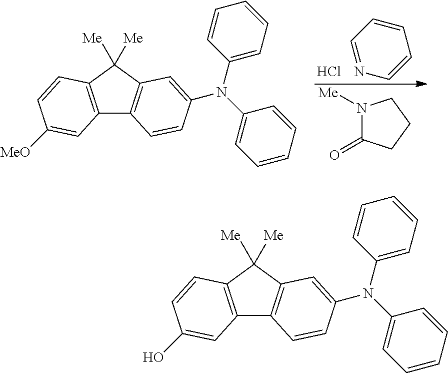

In a nitrogen atmosphere, a flask containing 7-(diphenylamino)-9,9′-dimethyl-9H-fluoren-3-ol (20.0 g), 2-bromo-1-fluoro-3-phenoxybenzene (15.6 g), potassium carbonate (18.3 g), and NMP (50 ml) was heated and stirred at a reflux temperature for four hours. After the reaction was stopped, the reaction liquid was cooled to room temperature, and water was added thereto. A precipitate thus precipitated was collected by suction filtration. The obtained precipitate was washed with water and then with Solmix and then purified by silica gel column chromatography (eluent: heptane/toluene=1/1 (volume ratio)) to obtain 30.0 g of 6-(2-bromo-3-phenoxyphenoxy)-9,9-dimethyl-N,N-diphenyl-9H-fluoren-2-amine (yield: 90.6%).

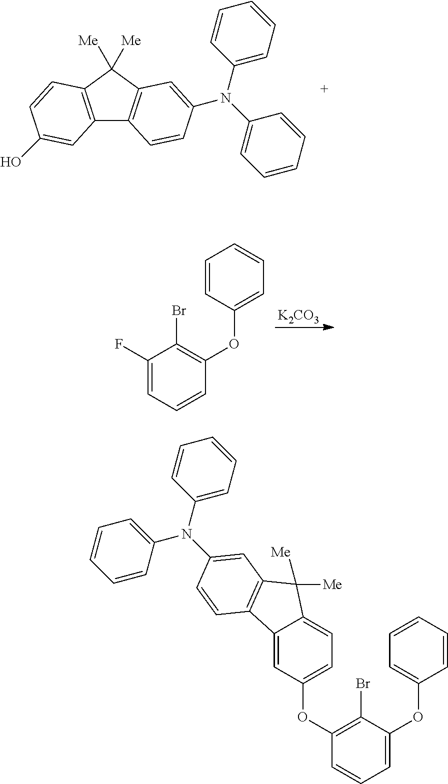

In a nitrogen atmosphere, a flask containing 6-(2-bromo-3-phenoxyphenoxy)-9,9-dimethyl-N,N-diphenyl-9H-fluoren-2-amine (28.0 g) and xylene (200 ml) was cooled to −30° C., and a 1.6 M n-butyllithium hexane solution (30.8 ml) was dropwise added thereto. After completion of the dropwise addition, the solution was stirred at room temperature for 0.5 hours. Thereafter, the reaction liquid was depressurized to distill off a component having a low boiling point. Thereafter, the residue was cooled to −30° C., and boron tribromide (16.8 g) was added thereto. The resulting solution was heated to room temperature, and stirred for 0.5 hours. Thereafter, the solution was cooled to 00° C., N-ethyl-N-isopropylpropan-2-amine (12.6 g) was added thereto, and the solution was stirred at room temperature for ten minutes. Subsequently, aluminum chloride (AlCl₃) (12.0 g) was added thereto, and the resulting mixture was heated at 90° C. for two hours. The reaction liquid was cooled to room temperature, and a potassium acetate aqueous solution was added thereto to stop the reaction. Thereafter, a precipitate thus precipitated was collected as a crude product 1 by suction filtration. The filtrate was extracted with ethyl acetate and dried with anhydrous sodium sulfate. Thereafter, the desiccant was removed, and a solvent was distilled off under reduced pressure to obtain a crude product 2. The crude products 1 and 2 were mixed with each other. The resulting mixture was reprecipitated several times with each of Solmix and heptane and then purified by NH2 silica gel column chromatography (eluent: ethyl acetate→toluene). Furthermore, sublimation purification was performed to obtain 6.4 g of a compound represented by formula (1-5101) (yield: 25.6%).

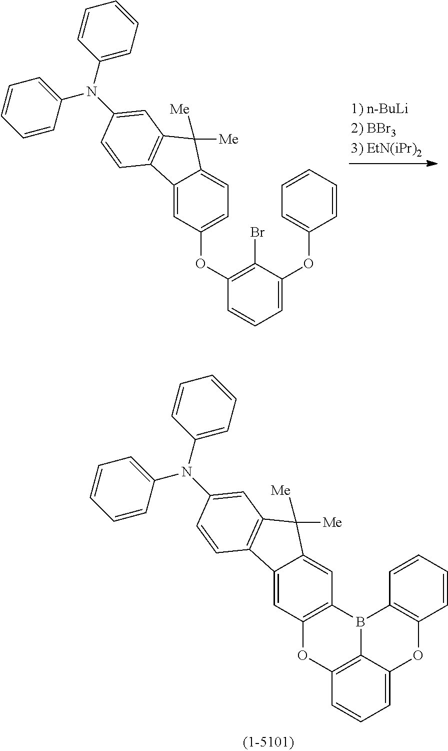 [SMILES: CC1(C)c2ccc(Oc3cccc(Oc4ccccc4)c3Br)cc2-c2ccc(N(c3ccccc3)c3ccccc3)cc21 ; CC1(C)c2cc(N(c3ccccc3)c3ccccc3)ccc2-c2cc3c(cc21)B1c2ccccc2Oc2cccc(c21)O3]

The structure of the compound thus obtained was identified by NMR measurement.

¹H-NMR (CDCl₃): δ=8.72 (d, 1H), 8.60 (s, 1H), 7.79-7.68 (m, 4H), 7.55 (d, 1H), 7.41 (t, 1H), 7.31-7.17 (m, 11H), 7.09-7.05 (m, 3H), 1.57 (s, 6H).

The compound thus obtained had a glass transition temperature (Tg) of 116.6° C.

[Measuring instrument: Diamond DSC (manufactured by PERKIN-ELMER); measurement conditions: cooling rate 200° C./min., heating rate 10° C./min.]

### Synthesis Example (8)

### Synthesis of Compound (1-5109): 15,15-dimethyl-N,N,5-triphenyl-5H,15H-9-oxa-5-aza-16b-boraindeno[1,2-b]naphtho[1,2,3-fg]anthracen-13-amine

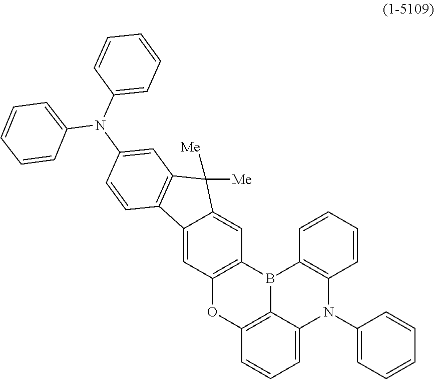 [SMILES: CC1(C)c2cc(N(c3ccccc3)c3ccccc3)ccc2-c2cc3c(cc21)B1c2ccccc2N(c2ccccc2)c2cccc(c21)O3]

In a nitrogen atmosphere, a flask containing 7-(diphenylamino)-9,9′-dimethyl-9H-fluoren-3-ol (100 g), 1-bromo-2-chloro-3-fluorobenzene (58.3 g), potassium carbonate (91.5 g), and NMP (500 ml) was heated and stirred at a reflux temperature for four hours. After the reaction was stopped, the reaction liquid was cooled to room temperature, and water was added thereto. A precipitate thus precipitated was collected by suction filtration. The obtained precipitate was washed with water and then with methanol and then purified by silica gel column chromatography (eluent: toluene) to obtain an intermediate 6-(3-bromo-2-chlorophenoxy)-9,9-dimethyl-N,N-diphenyl-9H-fluoren-2-amine (150 g).

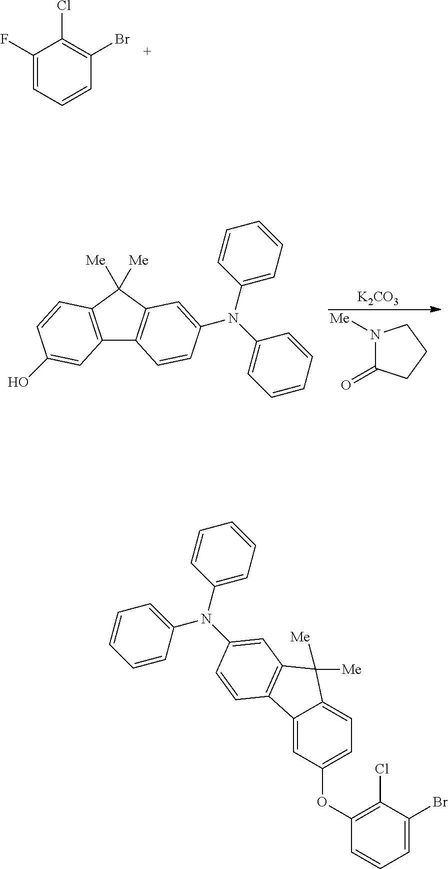

In a nitrogen atmosphere, a flask containing the intermediate 6-(3-bromo-2-chlorophenoxy)-9,9-dimethyl-N,N-diphenyl-9H-fluoren-2-amine (40 g), diphenylamine (12.5 g), Pd-132 (Johnson Matthey) (1.5 g), NaOtBu (17.0 g), and xylene (200 ml) was heated and stirred at 85° C. for two hours. The reaction liquid was cooled to room temperature, then water and toluene were added thereto, and the mixture was partitioned. A solvent of an organic layer was distilled off under reduced pressure. The obtained solid was washed several times with Solmix A-11 (trade name: Nippon Alcohol Trading Co., Ltd.) and then purified by silica gel column chromatography (eluent: toluene/heptane=1/2 (volume ratio)) to obtain an intermediate 6-(2-chloro-3-(diphenylamino) phenoxy)-9,9-dimethyl-N,N-diphenyl-9H-fluoren-2-amine (35.6 g).

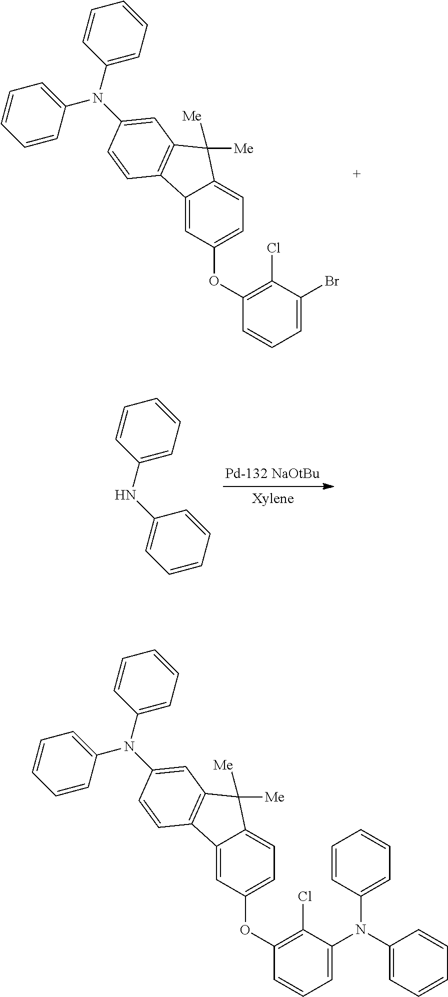 [SMILES: CC1(C)c2ccc(Oc3cccc(Br)c3Cl)cc2-c2ccc(N(c3ccccc3)c3ccccc3)cc21 ; c1ccc(Nc2ccccc2)cc1 ; CC1(C)c2ccc(Oc3cccc(N(c4ccccc4)c4ccccc4)c3Cl)cc2-c2ccc(N(c3ccccc3)c3ccccc3)cc21]

A flask containing the intermediate 6-(2-chloro-3-(diphenylamino) phenoxy)-9,9-dimethyl-N,N-diphenyl-9H-fluoren-2-amine (18.9 g) and toluene (150 ml) was heated to 70° C. in a nitrogen atmosphere, and the intermediate was completely dissolved therein. The flask was cooled to 0° C., and then a 2.6 M n-hexane solution of n-butyllithium (14.4 ml) was added thereto. The resulting solution was heated to 65° C. and stirred for three hours. Thereafter, the flask was cooled to −10° C., 2-isopropoxy-4,4,5,5-tetramethyl-1,3,2-dioxaborolane (13.4 g) was added thereto, and the resulting mixture was stirred at room temperature for two hours. Water and toluene were added thereto, and the mixture was partitioned. An organic layer was passed through a NH2 silica gel short column (eluent: toluene). A solvent was distilled off under reduced pressure to obtain an intermediate 6-(3-(diphenylamino)-2-(4,4,5,5-tetramethyl-1,3,2-dioxaborolan-2-yl) phenoxy)-9,9-dimethyl-N,N-diphenyl-9H-fluoren-2-amine (22 g).

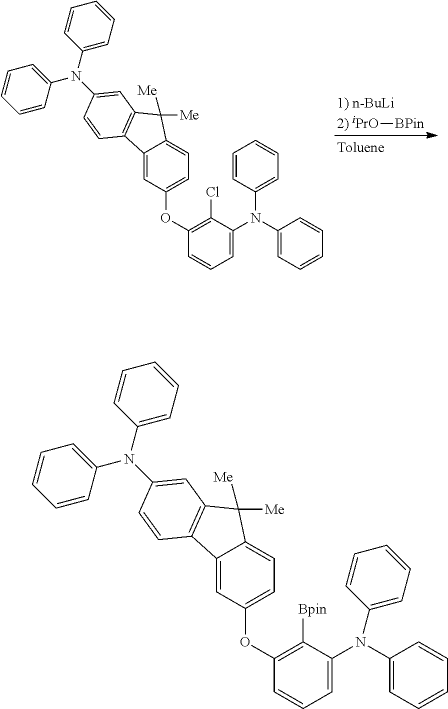

To a flask containing the intermediate 6-(3-(diphenylamino)-2-(4,4,5,5-tetramethyl-1,3,2-dioxaborolan-2-yl) phenoxy)-9,9-dimethyl-N,N-diphenyl-9H-fluoren-2-amine (21.5 g) and toluene (215 ml), aluminum chloride (19.2 g) and N,N-diisopropylethylamine (DIPEA) (3.7 g) were added. The resulting mixture was refluxed for three hours. Thereafter, the reaction mixture cooled to room temperature was poured into ice water (250 ml). Toluene was added thereto, and an organic layer was extracted. A solvent of the organic layer was distilled off under reduced pressure, and the obtained solid was subjected to short column purification (eluent: toluene/heptane=1/4 (volume ratio)) with NH2 silica gel and then reprecipitated several times with methanol. The obtained crude product was subjected to column purification with silica gel (eluent: toluene/heptane=1/2 (volume ratio)) and further subjected to sublimation purification to obtain a compound (4.1 g) represented by formula (1-5109).

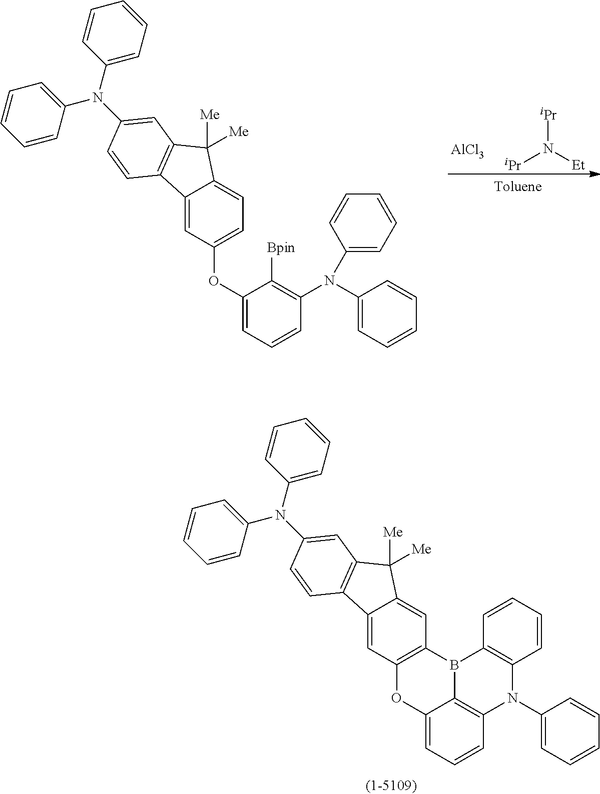

The structure of the compound thus obtained was identified by NMR measurement.

¹H-NMR (400 MHz, CDCl₃): δ=8.94 (dd, 1H), 8.70 (s, 1H), 7.74-7.69 (m, 4H), 7.62 (t, 1H), 7.53-7.47 (m, 2H), 7.38 (dd, 2H), 7.33-7.28 (m, 5H), 7.24 (d, 1H), 7.18 (dd, 4H), 7.09-7.05 (m, 4H), 6.80 (d, 1H), 6.30 (d, 1H), 1.58 (s, 6H).

### Synthesis Example (9)

### Synthesis of Compound (1-5001): 16,16,19,19-tetramethyl-N²,N²,N¹⁴,N¹⁴-tetraphenyl-16,19-dihydro-6,10-dioxa-17b-boraindeno[1,2-b]indeno[1′,2′:6,7]naphtho[1,2,3-fg]anthracene-2,14-diamine

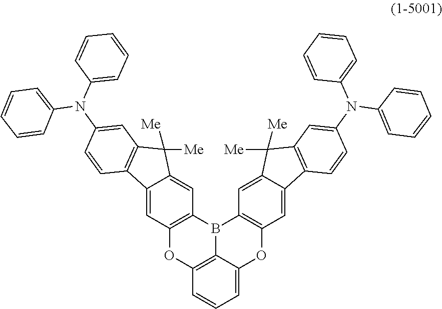 [SMILES: CC1(C)c2cc(N(c3ccccc3)c3ccccc3)ccc2-c2cc3c(cc21)B1c2cc4c(cc2Oc2cccc(c21)O3)-c1ccc(N(c2ccccc2)c2ccccc2)cc1C4(C)C]

In a nitrogen atmosphere, a flask containing 7-(diphenylamino)-9,9′-dimethyl-9H-fluoren-3-ol (14.1 g), 2-bromo-1,3-difluorobenzene (3.6 g), potassium carbonate (12.9 g), and NMP (30 ml) was heated and stirred at a reflux temperature for five hours. After the reaction was stopped, the reaction liquid was cooled to room temperature, and water was added thereto. A precipitate thus precipitated was collected by suction filtration. The obtained precipitate was washed with water and then with methanol and then purified by silica gel column chromatography (eluent: heptane/toluene mixed solvent) to obtain 6,6′-((2-bromo-1,3-phenylene) bis(oxy)) bis(9,9-dimethyl-N,N-diphenyl-9H-fluoren-2-amine) (12.6 g). At this time, the proportion of toluene in the eluent was gradually increased, and an intended product was thereby eluted.

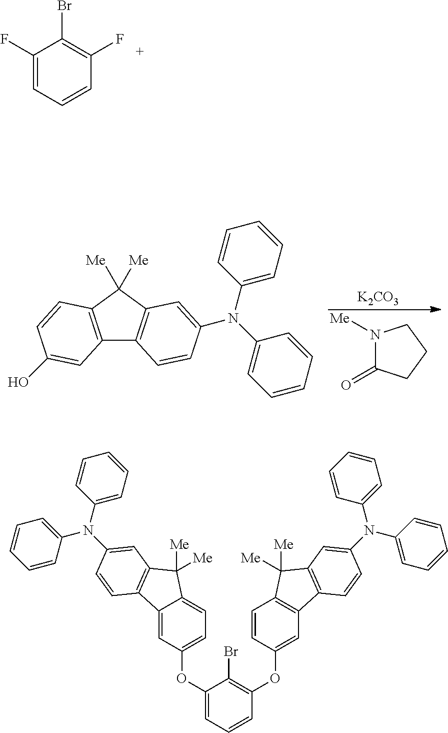

In a nitrogen atmosphere, a flask containing 6,6′-((2-bromo-1,3-phenylene) bis(oxy)) bis(9,9-dimethyl-N,N-diphenyl-9H-fluoren-2-amine) (11.0 g) and xylene (60.5 ml) was cooled to −40° C., and a 2.6 M n-butyllithium hexane solution (5.1 ml) was dropwise added thereto. After completion of the dropwise addition, the solution was stirred at this temperature for 0.5 hours. Thereafter, the solution was heated to 60° C., and stirred for three hours. Thereafter, the reaction liquid was depressurized to distill off a component having a low boiling point. Thereafter, the residue was cooled to −40° C., and boron tribromide (4.3 g) was added thereto. The solution was heated to room temperature, and stirred for 0.5 hours. Thereafter, the solution was cooled to 0° C., N-ethyl-N-isopropylpropan-2-amine (3.8 g) was added thereto, and the solution was heated and stirred at 125° C. for eight hours. The reaction liquid was cooled to room temperature, and a sodium acetate aqueous solution was added thereto to stop the reaction. Thereafter, toluene was added thereto, and the resulting solution was partitioned. An organic layer was purified with a silica gel short pass column, then by silica gel column chromatography (eluent: heptane/toluene=4/1 (volume ratio)), and further by activated carbon column chromatography (eluent: toluene) to obtain a compound represented by formula (1-5001) (1.2 q).

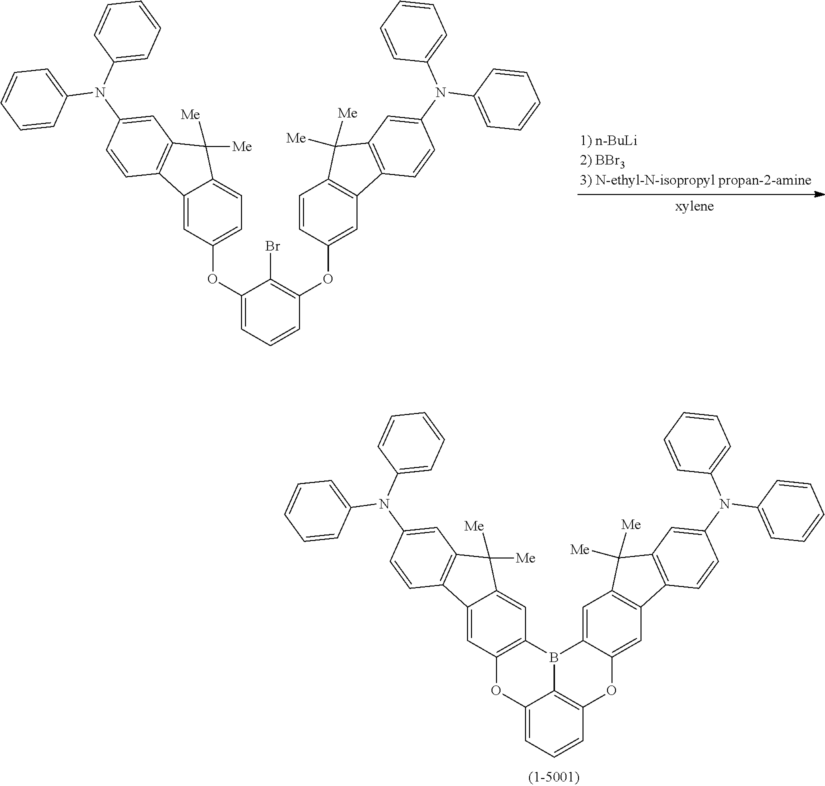 [SMILES: CC1(C)c2ccc(Oc3cccc(Oc4ccc5c(c4)-c4ccc(N(c6ccccc6)c6ccccc6)cc4C5(C)C)c3Br)cc2-c2ccc(N(c3ccccc3)c3ccccc3)cc21 ; CC1(C)c2cc(N(c3ccccc3)c3ccccc3)ccc2-c2cc3c(cc21)B1c2cc4c(cc2Oc2cccc(c21)O3)-c1ccc(N(c2ccccc2)c2ccccc2)cc1C4(C)C]

The structure of the compound thus obtained was identified by NMR measurement.

¹H-NMR (400 MHz, CDCl₃): δ=8.64 (s, 2H), 7.75 (m, 3H), 7.69 (d, 2H), 7.30 (t, 8H), 7.25 (s, 2H), 7.20 (m, 10H), 7.08 (m, 6H), 1.58 (s, 12H).

### Synthesis Example (10)

### Synthesis of Compound (1-5003): 16,16,19,19-tetramethyl-N²,N²,N¹⁴,N¹⁴-tetra-p-tolyl-16H,19H-6,10-dioxa-17b-boraindeno[1,2-b]indeno[1′,2′:6,7]naphtho[1,2,3-fg]anthracene-2,14-diamine

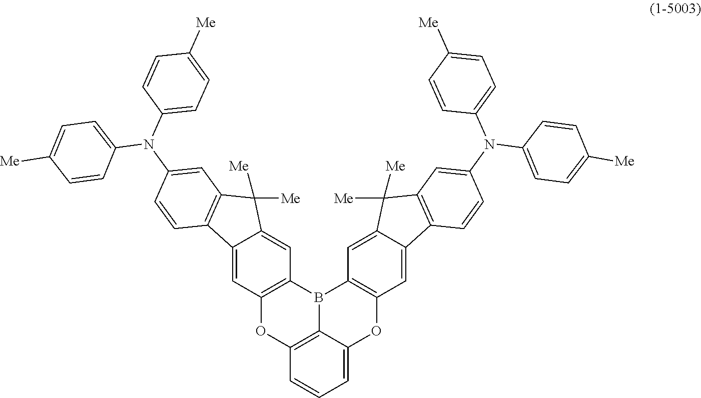 [SMILES: Cc1ccc(N(c2ccc(C)cc2)c2ccc3c(c2)C(C)(C)c2cc4c(cc2-3)Oc2cccc3c2B4c2cc4c(cc2O3)-c2ccc(N(c3ccc(C)cc3)c3ccc(C)cc3)cc2C4(C)C)cc1]

In a nitrogen atmosphere, a flask containing di-p-tolylamine (20.0 g), 2-chloro-6-methoxy-9,9-dimethyl-9H-fluorene (25.2 g), Pd-132 (Johnson Massey) (0.7 g), NaOtBu (14.0 g), and toluene (130 ml) was heated and refluxed for two hours. The reaction liquid was cooled to room temperature. Thereafter, water and toluene were added thereto, and the resulting mixture was partitioned. Subsequently, purification was performed by activated carbon column chromatography (eluent: toluene), and the purified product was further washed with Solmix to obtain 26.8 g of 4-(6-methoxy-9,9-dimethyl-N,N-di-p-tolyl-9H-fluoren-2-amine (yield: 66.1%).

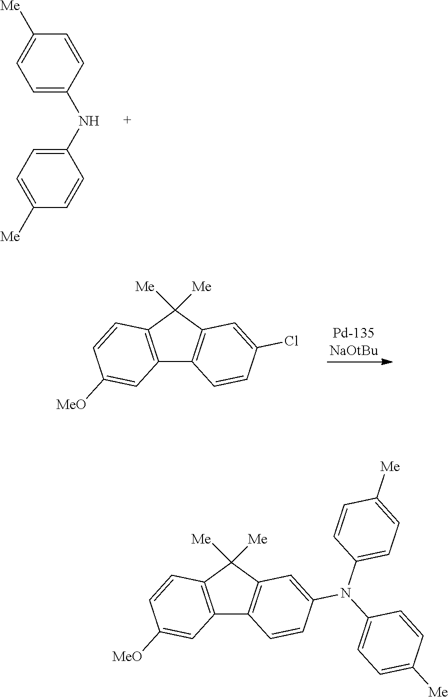 [SMILES: Cc1ccc(Nc2ccc(C)cc2)cc1 ; COc1ccc2c(c1)-c1ccc(Cl)cc1C2(C)C ; COc1ccc2c(c1)-c1ccc(N(c3ccc(C)cc3)c3ccc(C)cc3)cc1C2(C)C]

In a nitrogen atmosphere, 4-(6-methoxy-9,9-dimethyl-N,N-di-p-tolyl-9H-fluoren-2-amine (21.5 g), pyridine hydrochloride (29.6 g), and NMP (21.5 ml) were put in a flask and heated at 185° C. for five hours. After completion of heating, the reaction liquid was cooled to room temperature. Thereafter, water and toluene were added thereto, and the resulting solution was partitioned. Subsequently, an organic layer was dried with anhydrous sodium sulfate. Thereafter, the desiccant was removed, and a solvent was distilled off under reduced pressure to obtain a crude product. The crude product was purified with a short column (eluent: toluene) to obtain 20.8 g of 7-(di-p-tolylamino)-9,9-dimethyl-9H-fluoren-3-ol (yield: 100%).

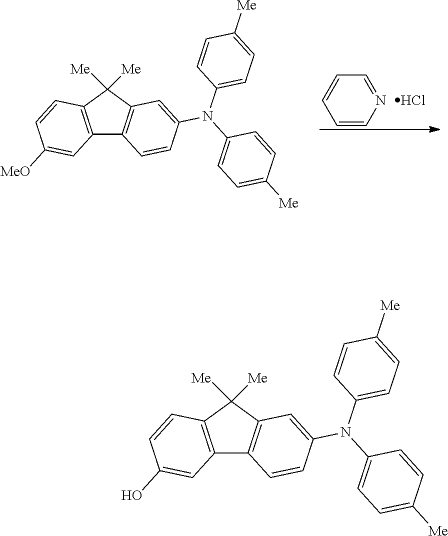 [SMILES: COc1ccc2c(c1)-c1ccc(N(c3ccc(C)cc3)c3ccc(C)cc3)cc1C2(C)C ; c1ccncc1 ; Cl ; Cc1ccc(N(c2ccc(C)cc2)c2ccc3c(c2)C(C)(C)c2ccc(O)cc2-3)cc1]

In a nitrogen atmosphere, a flask containing 7-(di-p-tolylamino)-9,9-dimethyl-9H-fluoren-3-ol (20.6 g), 2-bromo-1,3-difluorobenzene (4.9 g), potassium carbonate (17.5 g), and NMP (39 ml) was heated and stirred at a reflux temperature for two hours. After the reaction was stopped, the reaction liquid was cooled to room temperature, and water was added thereto. A precipitate thus precipitated was collected by suction filtration. The obtained precipitate was washed with water and then with Solmix and then purified by silica gel column chromatography (eluent: mixed solvent of heptane/toluene=2/1 (volume ratio) to obtain 17.3 g of 6,6′-((2-bromo-1,3-phenylene) bis(oxy)) bis(9,9-dimethyl-N,N-di-p-tolyl-9H-fluoren-2-amine) (yield: 70.7%).

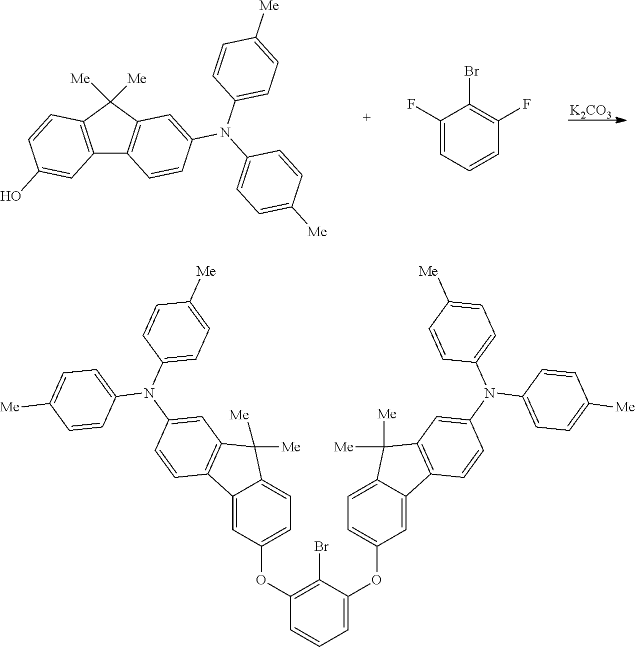

In a nitrogen atmosphere, a flask containing 6,6′-((2-bromo-1,3-phenylene) bis(oxy)) bis(9,9-dimethyl-N,N-di-p-tolyl-9H-fluoren-2-amine) (15.0 g) and xylene (100 ml) was cooled to −40° C., and a 1.6 M n-butyllithium hexane solution (10.7 ml) was dropwise added thereto. After completion of the dropwise addition, the solution was stirred at this temperature for 0.5 hours, and then heated to room temperature. Thereafter, the reaction liquid was depressurized to distill off a component having a low boiling point. Thereafter, the residue was cooled to −40° C., and boron tribromide (5.1 g) was added thereto. The solution was heated to room temperature, and stirred for 0.5 hours. Thereafter, the solution was cooled to 0° C., N-ethyl-N-isopropylpropan-2-amine (4.0 g) was added thereto, and the resulting solution was heated and stirred at 120° C. for five hours. The reaction liquid was cooled to room temperature, and a sodium acetate aqueous solution was added thereto to stop the reaction. Thereafter, toluene was added thereto, and the resulting solution was partitioned. An organic layer was purified with a silica gel short pass column (eluent: toluene) and then by NH2 silica gel column chromatography (eluent: ethyl acetate→toluene), and reprecipitation was performed several times with Solmix. Thereafter, purification was performed by silica gel column chromatography (eluent: heptane/toluene=3/1 (volume ratio)). Furthermore, sublimation purification was performed to obtain 1.5 g of a compound represented by formula (1-5003) (yield: 11%).

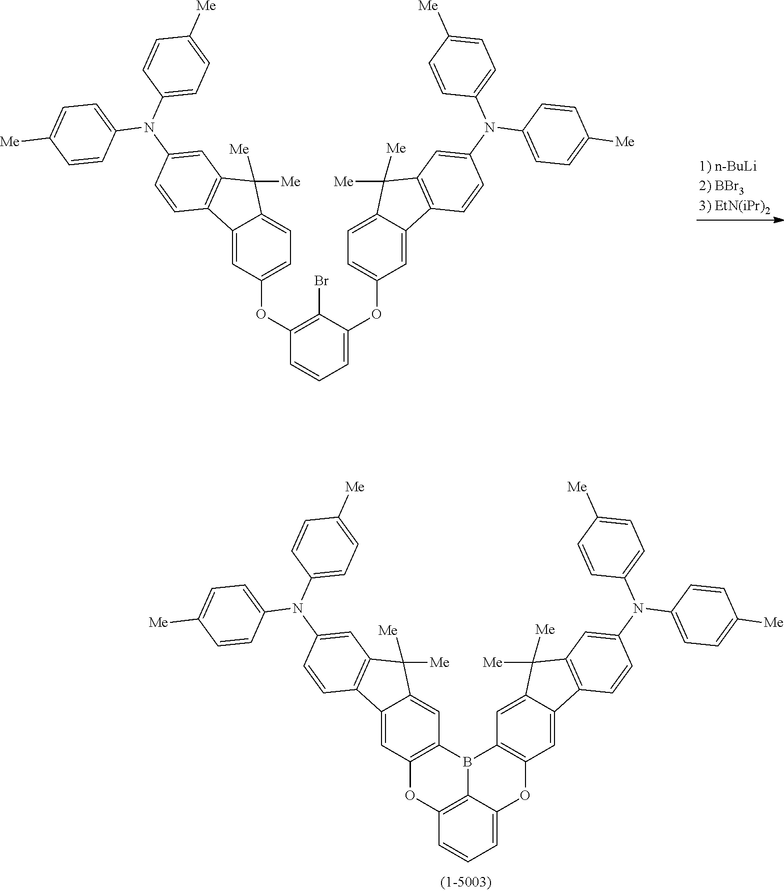 [SMILES: Cc1ccc(N(c2ccc(C)cc2)c2ccc3c(c2)C(C)(C)c2ccc(Oc4cccc(Oc5ccc6c(c5)-c5ccc(N(c7ccc(C)cc7)c7ccc(C)cc7)cc5C6(C)C)c4Br)cc2-3)cc1 ; Cc1ccc(N(c2ccc(C)cc2)c2ccc3c(c2)C(C)(C)c2cc4c(cc2-3)Oc2cccc3c2B4c2cc4c(cc2O3)-c2ccc(N(c3ccc(C)cc3)c3ccc(C)cc3)cc2C4(C)C)cc1]

The structure of the compound thus obtained was identified by NMR measurement.

¹H-NMR (CDCl₃): δ=8.62 (s, 2H), 7.74 (t, 1H), 7.72 (s, 2H), 7.65 (d, 2H), 7.25-7.06 (m, 20H), 7.00 (dd, 2H), 2.35 (s, 12H), 1.57 (s, 12H).

The obtained compound had a glass transition temperature (Tg) of 179.2° C.

[Measuring instrument: Diamond DSC (manufactured by PERKIN-ELMER); measurement conditions: cooling rate 200° C./min., heating rate 10° C./min.]

### Synthesis Example (11)

### Synthesis of Compound (1-5025): 8,16,16,19,19-pentamethyl-N²,N²,N¹⁴,N¹⁴-tetraphenyl-16H, 19H-6,10-dioxa-17b-boraindeno[1,2-b]indeno[1′,2′:6,7]naphtho[1,2,3-fg]anthracene-2,14-diamine

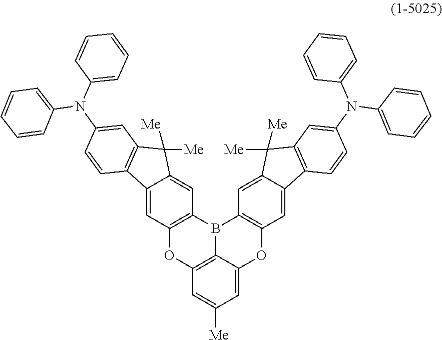 [SMILES: Cc1cc2c3c(c1)Oc1cc4c(cc1B3c1cc3c(cc1O2)-c1ccc(N(c2ccccc2)c2ccccc2)cc1C3(C)C)C(C)(C)c1cc(N(c2ccccc2)c2ccccc2)ccc1-4]

In a nitrogen atmosphere, a flask containing 7-(diphenylamino)-9,9′-dimethyl-9H-fluoren-3-ol (39.0 g), 1,3-difluoro-5-methylbenzene (6.6 g), tripotassium phosphate (54.8 g), and NMP (98 ml) was heated and stirred at a reflux temperature for 14 hours. After the reaction was stopped, the reaction liquid was cooled to room temperature, and water was added thereto. A precipitate thus precipitated was collected by suction filtration. The obtained precipitate was washed with water and then with Solmix and then purified by silica gel column chromatography (eluent: heptane/toluene=4/1-2/1 (volume ratio)) to obtain 41.0 g of 6,6′-((5-methyl-1,3-phenylene) bis(oxy)) bis(9,9-dimethyl-N,N-diphenyl-9H-fluoren-2-amine) (yield: 94.1%).

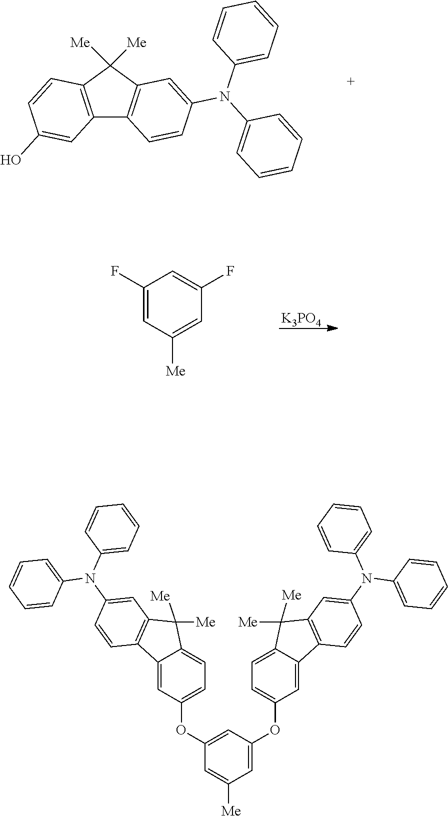

In a nitrogen atmosphere, a flask containing 6,6′-((5-methyl-1,3-phenylene) bis(oxy)) bis(9,9-dimethyl-N,N-diphenyl-9H-fluoren-2-amine) (41.0 g) and xylene (246 ml) was cooled to −10° C., and a 1.6 M n-butyllithium hexane solution (33.4 ml) was dropwise added thereto. After completion of the dropwise addition, the solution was stirred at this temperature for 0.5 hours. Thereafter, the solution was heated to 70° C., and stirred for two hours. Thereafter, the reaction liquid was depressurized to distill off a component having a low boiling point. Thereafter, the residue was cooled to −40° C., and boron tribromide (18.3 g) was added thereto. The resulting solution was heated to room temperature, and stirred for 0.5 hours. Thereafter, the solution was cooled to 0° C., N-ethyl-N-isopropylpropan-2-amine (12.6 g) was added thereto, and the solution was stirred at room temperature for ten minutes. Subsequently, aluminum chloride (AlCl₃) (13.0 g) was added thereto, and the resulting mixture was heated at 110° C. for three hours. The reaction liquid was cooled to room temperature, and a potassium acetate aqueous solution was added thereto to stop the reaction. Thereafter, toluene was added thereto, and the resulting solution was partitioned. An organic layer was purified with a silica gel short pass column (eluent: toluene) and then by NH2 silica gel column chromatography (eluent: ethyl acetate→toluene), and reprecipitation was performed several times with a mixed solvent of Solmix/heptane (volume ratio of 1/1). Thereafter, purification was performed by silica gel column chromatography (eluent: heptane/toluene=3/1 (volume ratio)). Furthermore, sublimation purification was performed to obtain 3.4 g of a compound represented by formula (1-5025) (yield: 8.2%).

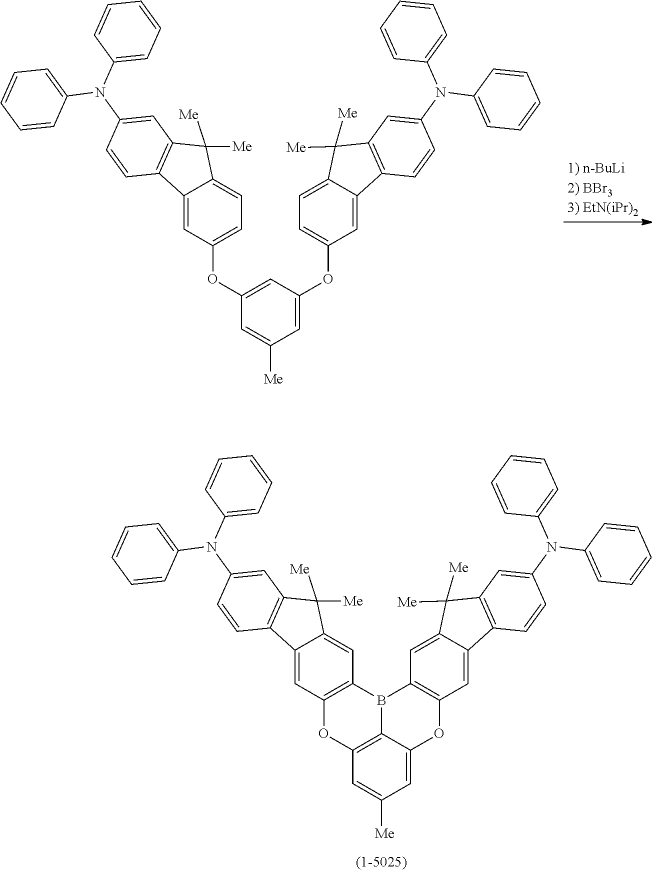 [SMILES: Cc1cc(Oc2ccc3c(c2)-c2ccc(N(c4ccccc4)c4ccccc4)cc2C3(C)C)cc(Oc2ccc3c(c2)-c2ccc(N(c4ccccc4)c4ccccc4)cc2C3(C)C)c1 ; Cc1cc2c3c(c1)Oc1cc4c(cc1B3c1cc3c(cc1O2)-c1ccc(N(c2ccccc2)c2ccccc2)cc1C3(C)C)C(C)(C)c1cc(N(c2ccccc2)c2ccccc2)ccc1-4]

The structure of the compound thus obtained was identified by NMR measurement.

¹H-NMR (CDCl₃): δ=8.62 (s, 2H), 7.72 (s, 2H), 7.68 (d, 2H), 7.30 (t, 8H), 7.25 (s, 2H), 7.18 (d, 8H), 7.08-7.03 (m, 8H), 2.58 (s, 3H), 1.57 (s, 12H).

The obtained compound had a glass transition temperature (Tg) of 182.5° C. [Measuring instrument: Diamond DSC (manufactured by PERKIN-ELMER); measurement conditions: cooling rate 200° C./min., heating rate 10° C./min.]

### Synthesis Example (12)

### Synthesis of Compound (1-5110): 5-([1,1′-biphenyl]-4-yl)-15,15-dimethyl-N,N,2-triphenyl-5H,15H-9-oxa-5-aza-16b-boraindeno[1,2-b]naphtho[1,2,3-fg]anthracen-13-amine

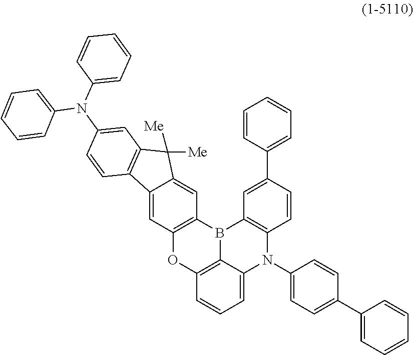 [SMILES: CC1(C)c2cc(N(c3ccccc3)c3ccccc3)ccc2-c2cc3c(cc21)B1c2cc(-c4ccccc4)ccc2N(c2ccc(-c4ccccc4)cc2)c2cccc(c21)O3]

In a nitrogen atmosphere, a flask containing 7-(diphenylamino)-9,9′-dimethyl-9H-fluoren-3-ol (9.0 g), 1,2-bromo-3-fluorobenzene (7.9 g), potassium carbonate (8.2 g), and NMP (45 ml) was heated and stirred at a reflux temperature for two hours. After the reaction was stopped, the reaction liquid was cooled to room temperature, and water was added thereto. A precipitate thus precipitated was collected by suction filtration. The obtained precipitate was washed with water and then with Solmix and then purified by silica gel column chromatography (eluent: heptane/toluene=3/1 (volume ratio)) to obtain 12.4 g of 6-(2,3-dibromophenoxy)-9,9-dimethyl-N,N-diphenyl-9H-fluoren-2-amine (yield: 84.8%).

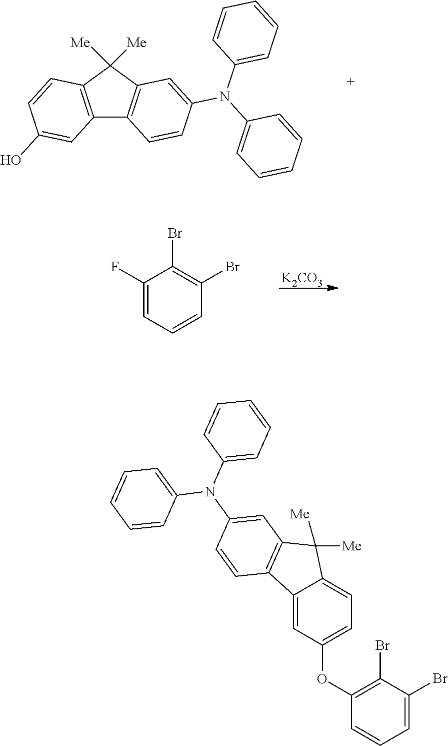

In a nitrogen atmosphere, a flask containing 6-(2,3-dibromophenoxy)-9,9-dimethyl-N,N-diphenyl-9H-fluoren-2-amine (10.0 g), di([1,1′-biphenyl]-4-yl) amine (5.3 g), palladium acetate (0.15 g), dicyclohexyl (2′,6′-diisopropoxy-[1,1′-biphenyl]-2-yl) phosphane (0.61 g), NaOtBu (2.4 g), and toluene (35 ml) was heated at 80° C. for six hours. The reaction liquid was cooled to room temperature. Thereafter, water and toluene were added thereto, and the resulting mixture was partitioned. Furthermore, purification was performed by silica gel column chromatography (eluent: heptane/toluene=2/1 (volume ratio)) to obtain 7.4 g of 6-(2-bromo-3-(di([1,1′-biphenyl]-4-yl) amino) phenoxy)-9,9-dimethyl-N,N-diphenyl-9H-fluoren-2-amine (yield: 53.1%).

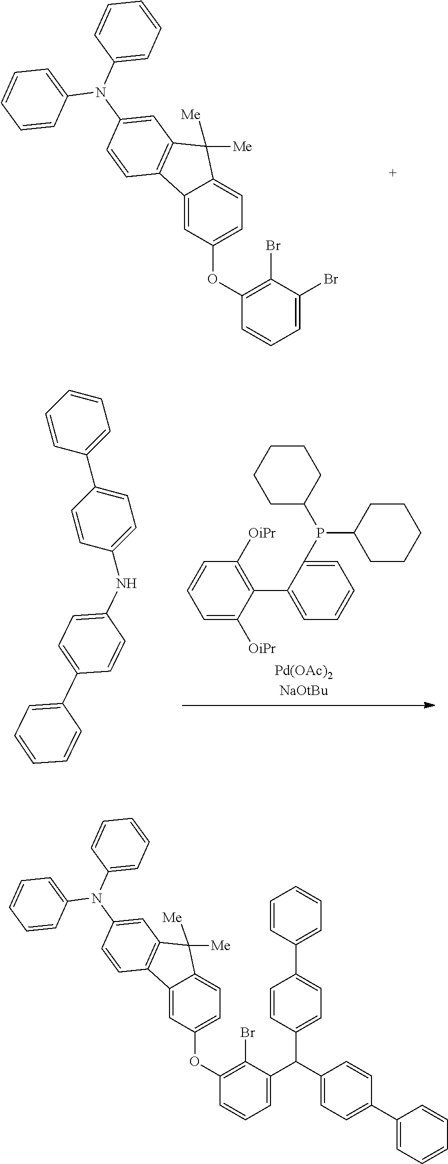 [SMILES: CC1(C)c2ccc(Oc3cccc(Br)c3Br)cc2-c2ccc(N(c3ccccc3)c3ccccc3)cc21 ; c1ccc(-c2ccc(Nc3ccc(-c4ccccc4)cc3)cc2)cc1 ; CC(C)Oc1cccc(OC(C)C)c1-c1ccccc1P(C1CCCCC1)C1CCCCC1 ; CC1(C)c2ccc(Oc3cccc(C(c4ccc(-c5ccccc5)cc4)c4ccc(-c5ccccc5)cc4)c3Br)cc2-c2ccc(N(c3ccccc3)c3ccccc3)cc21]

In a nitrogen atmosphere, 6-(2-bromo-3-(di([1,1′-biphenyl]-4-yl) amino) phenoxy)-9,9-dimethyl-N,N-diphenyl-9H-fluoren-2-amine (7.9 g) and tetrahydrofuran (42 ml) were put in a flask and cooled to −40° C. A 1.6 M n-butyllithium hexane solution (6 ml) was dropwise added thereto. After completion of the dropwise addition, the solution was stirred at this temperature for one hour. Thereafter, trimethylborate (1.7 g) was added thereto. The solution was heated to room temperature, and stirred for two hours. Thereafter, water (100 ml) was dropwise added slowly thereto. Subsequently, the reaction mixture was extracted with ethyl acetate and dried with anhydrous sodium sulfate. Thereafter, the desiccant was removed to obtain 7.0 g of dimethyl (2-(di([1,1′-biphenyl]-4-yl) amino)-6-((7-(diphenylamino)-9,9-dimethyl-9H-fluoren-3-yl) oxy) phenyl) boronate (yield: 100%).

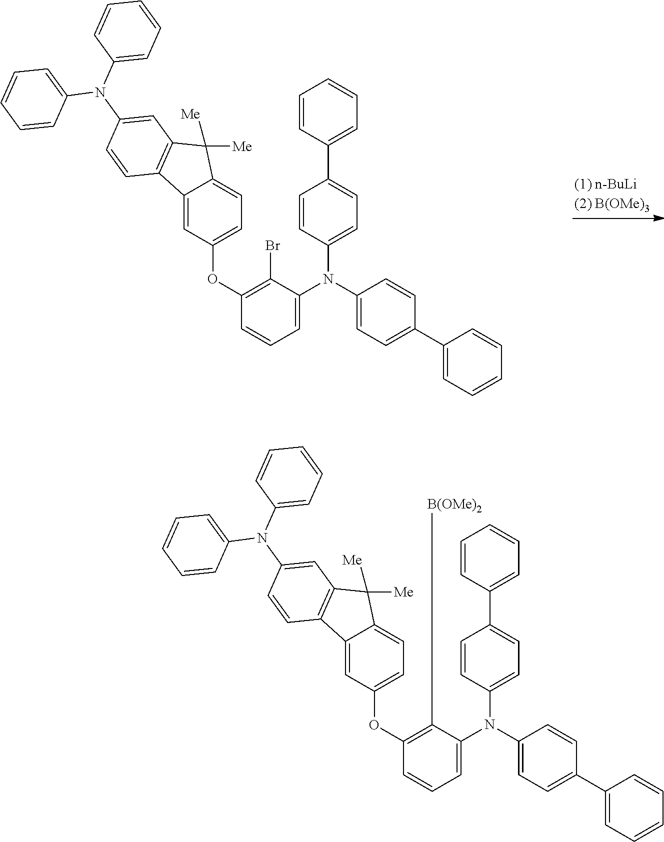

In a nitrogen atmosphere, dimethyl (2-(di([1,1′-biphenyl]-4-yl) amino)-6-((7-(diphenylamino)-9,9-dimethyl-9H-fluoren-3-yl) oxy) phenyl) boronate (6.5 g), aluminum chloride (10.3 g), and toluene (39 ml) were put in a flask and stirred for three minutes. Thereafter, N-ethyl-N-isopropylpropan-2-amine (2.5 g) was added thereto, and the resulting mixture was heated and stirred at 105° C. for one hour. After completion of heating, the reaction liquid was cooled, and ice water (20 ml) was added thereto. Thereafter, the reaction mixture was extracted with toluene. An organic layer was purified with a silica gel short pass column (eluent: toluene) and then by silica gel column chromatography (eluent: heptane/toluene=3/1 (volume ratio)). Thereafter, the purified product was reprecipitated with heptane, and the resulting precipitate was further purified with a NH2 silica gel column (solvent: heptane/toluene=1/1 (volume ratio)). Finally, sublimation purification was performed to obtain 0.74 g of a compound represented by formula (1-5110) (yield: 12.3%).

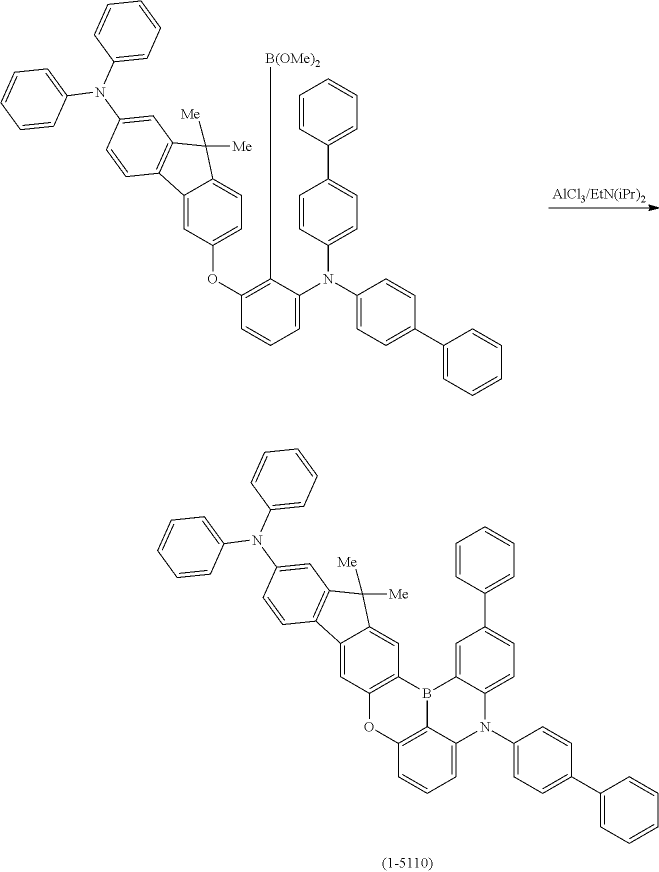

The structure of the compound thus obtained was identified by NMR measurement.

¹H-NMR (CDCl₃): δ=9.22 (s, 1H), 8.78 (s, 1H), 7.96 (d, 2H), 7.80-7.77 (m, 6H), 7.71 (d, 1H), 7.59-7.44 (m, 8H), 7.39 (t, 1H), 7.32-7.29 (m, 4H), 7.71 (d, 1H), 7.19 (dd, 4H), 7.12-7.06 (m, 4H), 7.00 (d, 1H), 6.45 (d, 1H), 1.57 (s, 6H).

The obtained compound had a glass transition temperature (Tg) of 165.6° C. [Measuring instrument: Diamond DSC (manufactured by PERKIN-ELMER); measurement conditions: cooling rate 200° C./min., heating rate 10° C./min.]

### Synthesis Example (13)

### Comparative Compound (3) was Synthesized According to the Method Described in JP 2013-080961 A (Manufacture Example 8 of Paragraph [0102])

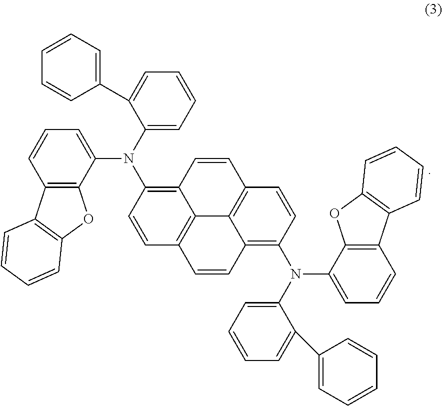 [SMILES: c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1]

### Synthesis Example (14)

### Compounds (2-301), (2-302), (2-383), (2-381), (2-382), (2-101), (2-202), and (2-303) were Synthesized According to the Method Described in JP 2011-006397 A

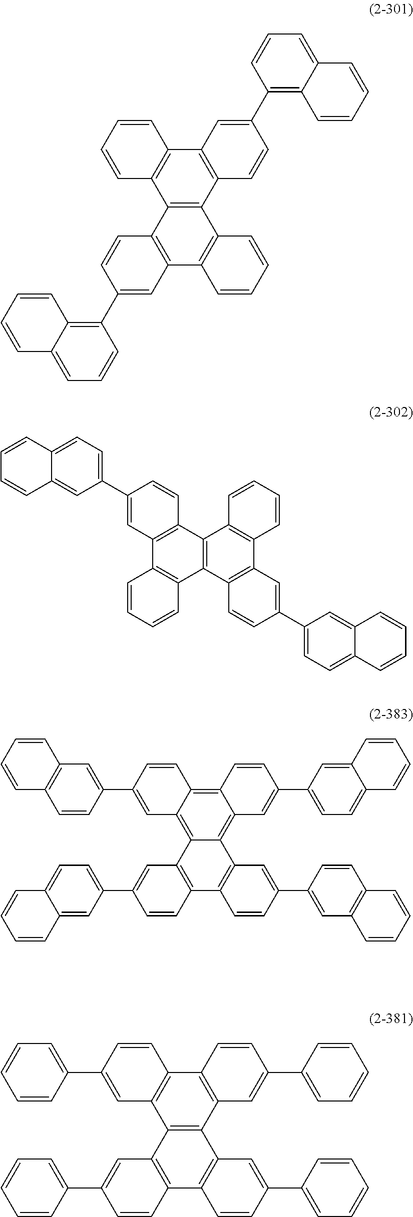

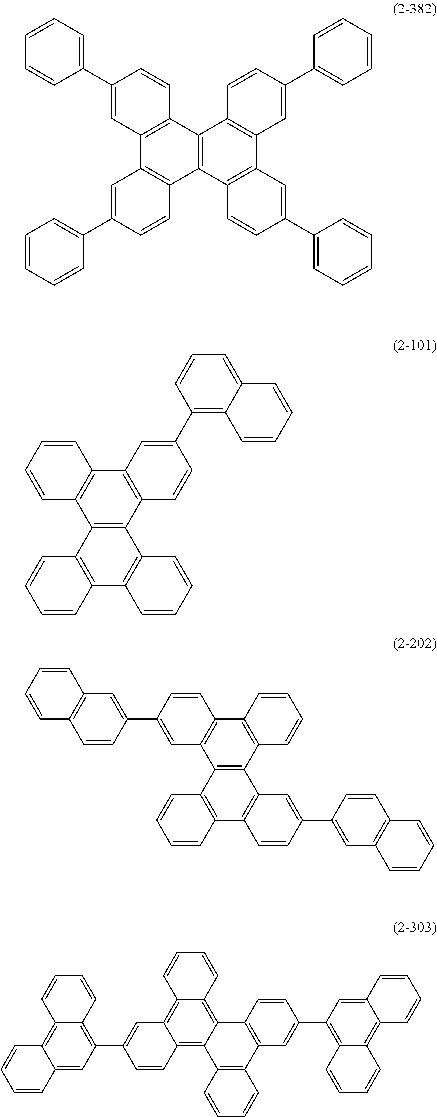

### Synthesis Example (15)

### Synthesis of Compound (2-401): 2-(dibenzo[g,p]chrysen-2-yl) naphtho[2,3-b]benzofuran

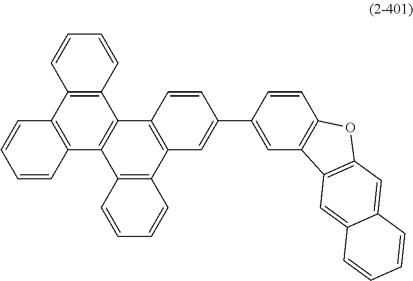 [SMILES: c1ccc2cc3c(cc2c1)oc1ccc(-c2ccc4c(c2)c2ccccc2c2c5ccccc5c5ccccc5c42)cc13]

In a nitrogen atmosphere, a 1.6 mol/L n-butyllithium/n-hexane solution (28 ml) was dropwise added to a THF (200 ml) suspension of 2-bromodibenzo[g,p]chrysene (14 g) at −70° C. The resulting solution was stirred for 0.5 h, and then 2-isopropoxy-4,4,5,5-tetramethyl-1,3,2-dioxaborolane (12.8 g) was added thereto. The resulting solution was heated to room temperature, and stirred for one hour. Thereafter, dilute hydrochloric acid was added thereto. Subsequently, toluene was added thereto, and extraction was performed. Oil obtained by concentrating an organic layer was purified by silica gel column chromatography (eluent: toluene) to obtain 2-(dibenzo[g,p]chrysen-2-yl)-4,4,5,5-tetramethyl-1,3,2-dioxaborolane (10 g).

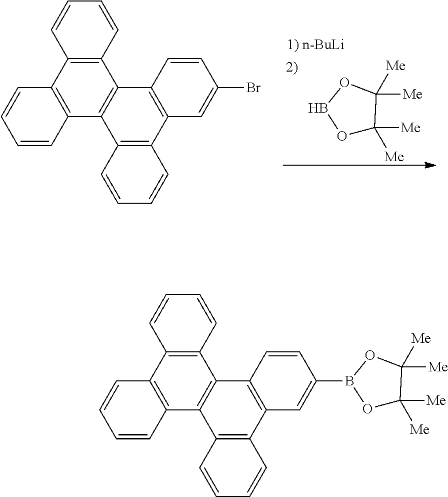

The structure of the compound thus obtained was identified by NMR measurement.

¹H-NMR (CDCl³): δ=1.44 (s, 12H), 7.61-7.71 (m, 6H), 8.03 (dd, 1H), 8.66-8.72 (m, 6H), 8.87 (dd, 1H), 9.19 (s, 1H).

In a nitrogen atmosphere, to 2-(dibenzo[g,p]chrysene-2-yl)-4,4,5,5-tetramethyl-1,3,2-dioxaborolane (1.0 g), 2-bromonaphtho[2,3-b]benzofuran (0.63 g), potassium phosphate (0.9 g), xylene (10 ml), t-butyl alcohol (3 ml), and water (2 ml), tetrakis(triphenylphosphine) palladium (62 mg) was added. The resulting mixture was heated and stirred at 110° C. for one hour. The mixture was cooled to room temperature. Thereafter, water and ethyl acetate were added thereto, and the resulting mixture was stirred for a while. Thereafter, a precipitate was filtered. This solid was recrystallized from chlorobenzene to obtain a compound (0.83 g) represented by formula (2-401).

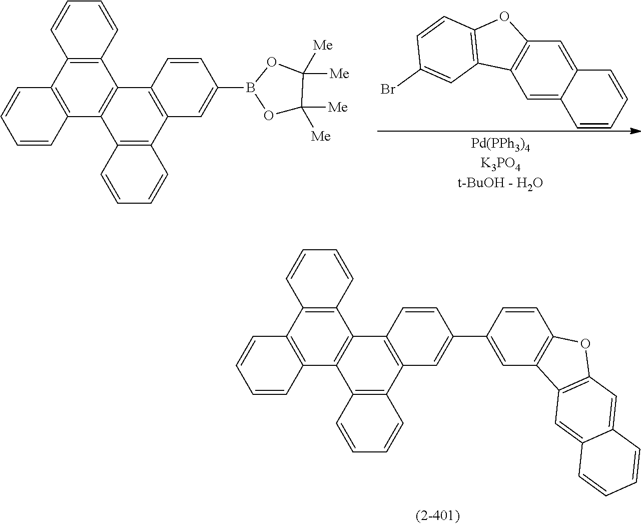 [SMILES: CC1(C)OB(c2ccc3c(c2)c2ccccc2c2c4ccccc4c4ccccc4c32)OC1(C)C ; Brc1ccc2oc3cc4ccccc4cc3c2c1 ; c1ccc2cc3c(cc2c1)oc1ccc(-c2ccc4c(c2)c2ccccc2c2c5ccccc5c5ccccc5c42)cc13]

The structure of the compound thus obtained was identified by NMR measurement.

¹H-NMR (CDCl₃): δ=7.51 (t, 1H), 7.56 (t, 1H), 7.65-7.76 (m, 7H), 7.98-8.02 (m, 4H), 8.09 (d, 1H), 8.51 (d, 1H), 8.56 (s, 1H), 8.73-8.79 (m, 5H), 8.83 (d, 1H), 8.88 (dd, 1H), 9.02 (d, 1H).

### Synthesis Example (16)

### Synthesis of Compound (2-427): 8-(dibenzo[g,p]chrysen-2-yl) naphtho[1,2-b]benzofuran

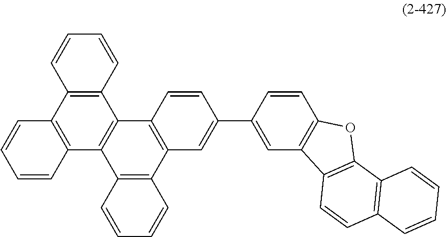 [SMILES: c1ccc2c(c1)ccc1c3cc(-c4ccc5c(c4)c4ccccc4c4c6ccccc6c6ccccc6c54)ccc3oc21]

Synthesis was performed according to Synthesis Example (15) except that 2-bromonaphtho[2,3-b]benzofuran was replaced with 8-bromonaphtho[1,2-b]benzofuran and tetrakis(triphenylphosphine) palladium was replaced with Pd-132 (Johnson Matthey) (16 mg) to obtain a compound (1.0 g) represented by formula (2-427).

The structure of the compound thus obtained was identified by NMR measurement.

¹H-NMR (CDCl₃): S=7.61 (t, 1H), 7.65-7.75 (m, 7H), 7.86 (d, 1H), 7.88 (d, 1H), 7.96 (dd, 1 h), 8.01 (dd, 1 h), 8.04 (d, 1H), 8.14 (d, 1H), 8.45 (dd, 1 h), 8.51 (d, 1H), 8.73-8.76 (m, 4H), 8.78 (dd, 1H), 8.83 (d, 1H), 8.87 (dd, 1H), 9.03 (d, 1H).

### Synthesis Example (17)

### Synthesis of Compound (2-419): 3-(dibenzo[g,p]chrysen-2-yl) naphtho[2,3-b]benzofuran

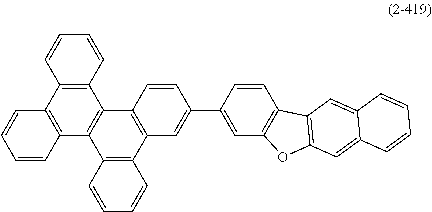 [SMILES: c1ccc2cc3c(cc2c1)oc1cc(-c2ccc4c(c2)c2ccccc2c2c5ccccc5c5ccccc5c42)ccc13]

Synthesis was performed according to Synthesis Example (15) except that 2-bromonaphtho[2,3-b]benzofuran was replaced with 3-bromonaphtho[2,3-b]benzofuran and tetrakis(triphenylphosphine) palladium was replaced with Pd-132 (Johnson Matthey) (16 mg) to obtain a compound (1.0 g) represented by formula (2-419).

The structure of the compound thus obtained was identified by NMR measurement.

¹H-NMR (CDCl₃): δ=7.49-7.57 (m, 2H), 7.64-7.76 (m, 6H), 7.89 (dd, 1H), 7.98-8.02 (m, 3H), 8.05 (d, 1H), 8.07 (d, 1H), 8.22 (d, 1H), 8.49 (s, 1H), 8.73-8.77 (m, 5H), 8.82-8.86 (m, 2H), 9.04 (d, 1H).

### Synthesis Example (18)

### Synthesis of Compound (2-411): 9-(dibenzo[g,p]chrysen-2-yl) naphtho[1,2-b]benzofuran

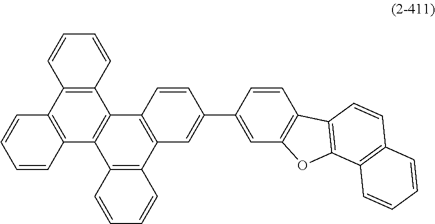 [SMILES: c1ccc2c(c1)ccc1c3ccc(-c4ccc5c(c4)c4ccccc4c4c6ccccc6c6ccccc6c54)cc3oc21]

Synthesis was performed according to Synthesis Example (15) except that 2-bromonaphtho[2,3-b]benzofuran was replaced with 9-bromonaphtho[1,2-b]benzofuran and tetrakis(triphenylphosphine) palladium was replaced with Pd-132 (Johnson Matthey) (16 mg) to obtain a compound (1.0 g) represented by formula (2-411).

The structure of the compound thus obtained was identified by NMR measurement.

¹H-NMR (CDCl₃): δ=7.60 (t, 1H), 7.64-7.76 (m, 7H), 7.84 (d, 1H), 7.92 (dd, 1H), 8.01-8.04 (m, 2H), 8.08 (d, 1H), 8.16 (d, 1H), 8.20 (d, 1H), 8.51 (dd, 1H), 8.73-8.76 (m, 4H), 8.77 (dd, 1H), 8.83 (d, 1H), 8.86 (dd, 1H), 9.05 (d, 1H).

### Synthesis Example (19)

### Synthesis of Compound (2-660): 9-(4-(dibenzo[g,p]chrysen-2-yl) naphthalen-1-yl)-9H-carbazole

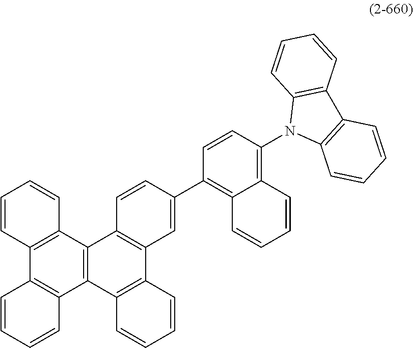 [SMILES: c1ccc2c(-n3c4ccccc4c4ccccc43)ccc(-c3ccc4c(c3)c3ccccc3c3c5ccccc5c5ccccc5c43)c2c1]

Synthesis was performed according to Synthesis Example (15) except that 2-bromonaphtho[2,3-b]benzofuran was replaced with 9-(4-bromonaphthalen-1-yl)-9H-carbazole and tetrakis(triphenylphosphine) palladium was replaced with dichlorobis(triphenylphosphine) palladium (II) to obtain a compound (0.9 g) represented by formula (2-660).

The structure of the compound thus obtained was identified by NMR measurement.

¹H-NMR (CDCl₃): δ=7.16 (d, 2H), 7.32-7.43 (m, 6H), 7.54 (t, 1H), 7.66-7.76 (m, 6H), 7.78 (d, 1H), 7.83 (d, 1H), 7.91 (dd, 1H), 8.22-8.26 (m, 3H), 8.75-8.78 (m, 5H), 8.86 (dd, 1H), 8.91 (d, 1H), 8.96 (d, 1H).

### Synthesis Example (20)

### Synthesis of Compound (2-643): 5-(dibenzo[g,p]chrysen-2-yl)-7,9-diphenyl-7H-benzo[c]carbazole

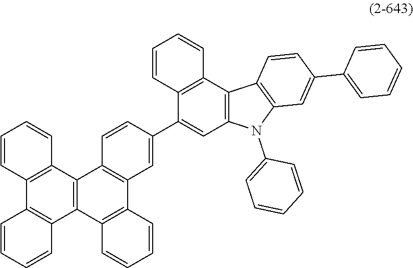 [SMILES: c1ccc(-c2ccc3c4c5ccccc5c(-c5ccc6c(c5)c5ccccc5c5c7ccccc7c7ccccc7c65)cc4n(-c4ccccc4)c3c2)cc1]

In a nitrogen atmosphere, to 2-bromodibenzo[g,p]chrysene (0.63 g), 7,9-diphenyl-5-(4,4,5,5-tetramethyl-1,3,2-dioxaborolan-2-yl)-7H-benzo[c]carbazole (0.8 g), potassium phosphate (0.7 g), xylene (10 ml), t-butyl alcohol (3 ml), and water (2 ml), dichlorobis(triphenylphosphine) palladium (23 mg) was added. The resulting mixture was heated and stirred at 110° C. for one hour. The resulting mixture was cooled to room temperature. Thereafter, water and then toluene were added thereto. Oil obtained by concentrating an organic layer was purified by silica gel column chromatography (eluent: toluene/heptane=3/7 (volume ratio)). Heptane was added to the obtained oil for reprecipitation to obtain a compound (0.7 g) represented by formula (2-643).

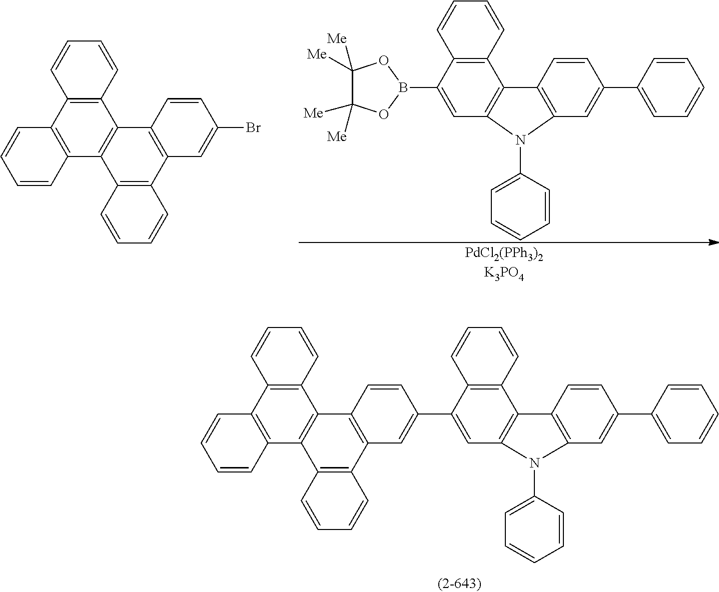 [SMILES: Brc1ccc2c(c1)c1ccccc1c1c3ccccc3c3ccccc3c21 ; CC1(C)OB(c2cc3c(c4ccccc24)c2ccc(-c4ccccc4)cc2n3-c2ccccc2)OC1(C)C ; c1ccc(-c2ccc3c4c5ccccc5c(-c5ccc6c(c5)c5ccccc5c5c7ccccc7c7ccccc7c65)cc4n(-c4ccccc4)c3c2)cc1]

The structure of the compound thus obtained was identified by NMR measurement.

¹H-NMR (CDCl₃): δ=7.43-7.57 (m, 8H), 7.63-7.73 (m, 10H), 7.80 (t, 1H), 7.90 (d, 1H), 7.95-7.97 (m, 2H), 8.04 (dd, 1H), 8.72-8.83 (m, 8H), 8.99-9.01 (m, 2H).

### Synthesis Example (21)

### Synthesis of Compound (2-662): 9-(dibenzo[g,p]chrysen-2-yl)-3,6-diphenyl-9H-carbazole

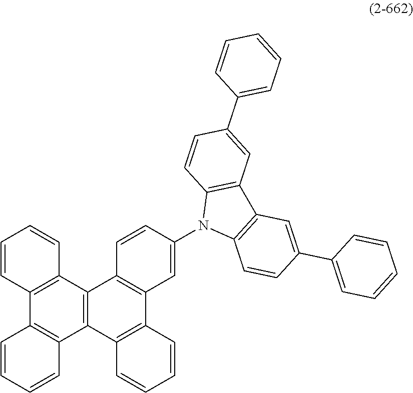 [SMILES: c1ccc(-c2ccc3c(c2)c2cc(-c4ccccc4)ccc2n3-c2ccc3c(c2)c2ccccc2c2c4ccccc4c4ccccc4c32)cc1]

In a nitrogen atmosphere, to 2-bromodibenzo[g,p]chrysene (0.6 g), 3,6-diphenyl-9H-carbazole (0.52 g), sodium t-butoxide (0.2 g), and 1,2,4-trimethylbenzene (10 ml), bis(dibenzylideneacetone) palladium (25 mg) and tri-t-butylphosphine (27 mg) were added. The resulting mixture was heated and stirred at 160° C. for one hour. The resulting mixture was cooled to room temperature. Thereafter, water and then ethyl acetate were added thereto. Oil obtained by concentrating an organic layer was purified by silica gel column chromatography (eluent: toluene/heptane=3/7 (volume ratio)). The obtained oil was dissolved in ethyl acetate, and heptane was added thereto for reprecipitation to obtain a compound (0.7 g) represented by formula (2-662).

The structure of the compound thus obtained was identified by NMR measurement.

¹H-NMR (CDCl₃): δ=7.37 (t, 2H), 7.51 (t, 4H), 7.66-7.78 (m, 14H), 7.89 (dd, 1H), 8.48 (d, 2H), 8.65-8.67 (m, 1H), 8.75-8.78 (m, 4H), 8.80 (dd, 1H), 8.96 (d, 1H), 8.98 (d, 1H).

Other compounds of the present invention can be synthesized by a method according to Synthesis Examples described above by appropriately changing the compounds of raw materials.

Hereinafter, Examples of an organic EL element using the compound of the present invention will be described in order to describe the present invention in more detail, but the present invention is not limited thereto.

Organic EL elements according to Examples 1 to 20, Examples 21 to 24 and Comparative Examples 1 to 6 were manufactured. Voltage (V), emission wavelength (nm), CIE chromaticity (x, y), and external quantum efficiency (%) thereof as characteristics at the time of emission of 1000 cd/m² were measured.

The quantum efficiency of a luminescent element includes an internal quantum efficiency and an external quantum efficiency. However, the internal quantum efficiency indicates a ratio at which external energy injected as electrons (or holes) into a light emitting layer of a luminescent element is purely converted into photons. Meanwhile, the external quantum efficiency is a value calculated based on the amount of photons emitted to an outside of the luminescent element. A part of the photons generated in the light emitting layer is absorbed or reflected continuously inside the luminescent element, and is not emitted to the outside of the luminescent element. Therefore, the external quantum efficiency is lower than the internal quantum efficiency.

A method for measuring the external quantum efficiency is as follows. Using a voltage/current generator R6144 manufactured by Advantest Corporation, a voltage at which luminance of an element was 1000 cd/m² was applied to cause the element to emit light. Using a spectral radiance meter SR-3AR manufactured by TOPCON Co., spectral radiance in a visible light region was measured from a direction perpendicular to a light emitting surface. Assuming that the light emitting surface is a perfectly diffusing surface, a numerical value obtained by dividing a spectral radiance value of each measured wavelength component by wavelength energy and multiplying the obtained value by n is the number of photons at each wavelength. Subsequently, the number of photons was integrated in the observed entire wavelength region, and this number was taken as the total number of photons emitted from the element. A numerical value obtained by dividing an applied current value by an elementary charge is taken as the number of carriers injected into the element. The external quantum efficiency is a numerical value obtained by dividing the total number of photons emitted from the element by the number of carriers injected into the element.

The following Tables 1a, 1b and 1c indicate a material composition of each layer and EL characteristic data in organic EL elements manufactured according to Examples 1 to 20, Examples 21 to 24 and Comparative Examples 1 to 6.

TABLE 1a

| Hole | Hole | Hole | Hole | | | Electron | Electron | Negative |
|---|---|---|---|---|---|---|---|---|
| injection | injection | transport | transport | Light emitting layer | | transport | transport | electrode |
| layer 1 | layer 2 | layer 1 | layer 2 | (25 nm) | | layer 1 | layer 2 | (1 nm/ |
| (40 nm) | (5 nm) | (15 nm) | (10 nm) | Host | Dopant | (5 nm) | (25 nm) | 100 nm) |

Examples

| | | | | | | | | | |
|---|---|---|---|---|---|---|---|---|---|
| 1 | HI | HAT-CN | HT-1 | HT-2 | 2-301 | 1-2619 | ET-1 | ET-3 + Liq | Liq/MgAg |
| 2 | HI | HAT-CN | HT-1 | HT-3 | 2-301 | 1-2619 | ET-1 | ET-3 + Liq | Liq/MgAg |
| 3 | HI | HAT-CN | HT-1 | HT-2 | 2-302 | 1-2619 | ET-1 | ET-3 + Liq | Liq/MgAg |
| 4 | HI | HAT-CN | HT-1 | HT-3 | 2-302 | 1-2619 | ET-1 | ET-3 + Liq | Liq/MgAg |
| 5 | HI | HAT-CN | HT-1 | HT-2 | 2-302 | 1-2621 | ET-2 | ET-4 + Liq | Liq/MgAg |
| 6 | HI | HAT-CN | HT-1 | HT-2 | 2-383 | 1-2621 | ET-2 | ET-4 + Liq | Liq/MgAg |
| 7 | HI | HAT-CN | HT-1 | HT-2 | 2-381 | 1-2621 | ET-2 | ET-4 + Liq | Liq/MgAg |
| 8 | HI | HAT-CN | HT-1 | HT-3 | 2-381 | 1-2619 | ET-1 | ET-3 + Liq | Liq/MgAg |
| 9 | HI | HAT-CN | HT-1 | HT-3 | 2-382 | 1-2619 | ET-1 | ET-3 + Liq | Liq/MgAg |
| 10 | HI | HAT-CN | HT-1 | HT-3 | 2-101 | 1-2619 | ET-1 | ET-3 + Liq | Liq/MgAg |
| 11 | HI | HAT-CN | HT-1 | HT-3 | 2-202 | 1-2619 | ET-1 | ET-3 + Liq | Liq/MgAg |
| 12 | HI | HAT-CN | HT-1 | HT-3 | 2-303 | 1-2619 | ET-1 | ET-3 + Liq | Liq/MgAg |
| 13 | HI | HAT-CN | HT-1 | HT-2 | 2-401 | 1-2619 | ET-1 | ET-3 + Liq | Liq/MgAg |
| 14 | HI | HAT-CN | HT-1 | HT-3 | 2-401 | 1-2619 | ET-1 | ET-3 + Liq | Liq/MgAg |
| 15 | HI | HAT-CN | HT-1 | HT-2 | 2-427 | 1-2619 | ET-1 | ET-3 + Liq | Liq/MgAg |
| 16 | HI | HAT-CN | HT-1 | HT-2 | 2-419 | 1-2619 | ET-1 | ET-3 + Liq | Liq/MgAg |
| 17 | HI | HAT-CN | HT-1 | HT-2 | 2-411 | 1-2619 | ET-1 | ET-3 + Liq | Liq/MgAg |
| 18 | HI | HAT-CN | HT-1 | HT-3 | 2-660 | 1-2619 | ET-1 | ET-3 + Liq | Liq/MgAg |
| 19 | HI | HAT-CN | HT-1 | HT-3 | 2-643 | 1-2619 | ET-1 | ET-3 + Liq | Liq/MgAg |
| 20 | HI | HAT-CN | HT-1 | HT-2 | 2-662 | 1-2619 | ET-1 | ET-3 + Liq | Liq/MgAg |

| | | | |
|---|---|---|---|
| Wavelength | Chromaticity | Voltage | |
| (nm) | (x, y) | (V) | External quantum efficiency |

Examples

| | | | | |
|---|---|---|---|---|
| 1 | 462 | (0.132, 0.085) | 4.0 | 6.7 |
| 2 | 461 | (0.132, 0.082) | 4.1 | 7.2 |
| 3 | 464 | (0.129, 0.108) | 3.9 | 5.8 |
| 4 | 463 | (0.129, 0.104) | 4.0 | 6.0 |
| 5 | 466 | (0.124, 0.118) | 3.9 | 6.0 |
| 6 | 467 | (0.123, 0.121) | 3.9 | 5.8 |
| 7 | 466 | (0.126, 0.108) | 4.1 | 5.9 |
| 8 | 463 | (0.130, 0.095) | 4.3 | 6.6 |
| 9 | 462 | (0.132, 0.100) | 4.1 | 6.0 |
| 10 | 462 | (0.131, 0.087) | 4.4 | 6.4 |
| 11 | 464 | (0.127, 0.101) | 4.3 | 7.1 |
| 12 | 462 | (0.130, 0.093) | 4.4 | 6.6 |
| 13 | 463 | (0.132, 0.087) | 3.9 | 6.2 |
| 14 | 463 | (0.130, 0.092) | 3.8 | 6.5 |
| 15 | 462 | (0.134, 0.079) | 3.8 | 6.8 |
| 16 | 463 | (0.130, 0.101) | 4.0 | 6.4 |
| 17 | 462 | (0.133, 0.087) | 4.0 | 6.2 |
| 18 | 462 | (0.132, 0.082) | 4.3 | 7.9 |
| 19 | 463 | (0.130, 0.095) | 3.4 | 5.9 |
| 20 | 461 | (0.136, 0.078) | 4.4 | 5.4 |

TABLE 1b

| Hole | Hole | Hole | Hole | | | Electron | Electron | Negative |
|---|---|---|---|---|---|---|---|---|
| injection | injection | transport | transport | Light emitting layer | | transport | transport | electrode |
| layer 1 | layer 2 | layer 1 | layer 2 | (25 nm) | | layer 1 | layer 2 | (1 nm/ |
| (40 nm) | (5 nm) | (15 nm) | (10 nm ) | Host | Dopant | (5 nm) | (25 nm) | 100 nm) |

Comparative Examples

| | | | | | | | | | |
|---|---|---|---|---|---|---|---|---|---|
| 1 | HI | HAT-CN | HT-1 | HT-2 | 2-301 | Compound (3) | ET-1 | ET-3 + Liq | Liq/MgAg |
| 2 | HI | HAT-CN | HT-1 | HT-3 | 2-301 | Compound (3) | ET-1 | ET-3 + Liq | Liq/MgAg |
| 3 | HI | HAT-CN | HT-1 | HT-2 | 2-302 | Compound (3) | ET-1 | ET-3 + Liq | Liq/MgAg |
| 4 | HI | HAT-CN | HT-1 | HT-3 | 2-302 | Compound (3) | ET-1 | ET-3 + Liq | Liq/MgAg |
| 5 | HI | HAT-CN | HT-1 | HT-3 | 2-381 | Compound (3) | ET-1 | ET-3 + Liq | Liq/MgAg |
| 6 | HI | HAT-CN | HT-1 | HT-3 | 2-401 | Compound (3) | ET-1 | ET-3 + Liq | Liq/MgAg |

| | | | |
|---|---|---|---|
| Wavelength | Chromaticity | Voltage | |
| (nm) | (x, y) | (V) | External quantum efficiency |

Comparative Examples

| | | | | |
|---|---|---|---|---|
| 1 | 460 | (0.134, 0.120) | 4.1 | 4.8 |
| 2 | 460 | (0.134, 0.120) | 4.1 | 4.8 |
| 3 | 462 | (0.133, 0.152) | 3.9 | 4.5 |
| 4 | 462 | (0.134, 0.145) | 4.0 | 4.8 |
| 5 | 460 | (0.133, 0.123) | 4.5 | 4.1 |
| 6 | 460 | (0.135, 0.116) | 4.1 | 4.1 |

TABLE 1c

| Hole | Hole | Hole | Hole | | | Electron | Electron | Negative |
|---|---|---|---|---|---|---|---|---|
| injection | injection | transport | transport | Light emitting layer | | transport | transport | electrode |
| layer 1 | layer 2 | layer 1 | layer 2 | (25 nm) | | layer 1 | layer 2 | (1 nm/ |
| (40 nm) | (5 nm) | (15 nm) | (10 nm) | Host | Dopant | (5 nm) | (25 nm) | 100 nm) |

Examples

| | | | | | | | | | |
|---|---|---|---|---|---|---|---|---|---|
| 21 | HI | HAT-CN | HT-1 | HT-2 | 2-301 | 1-5101 | ET-1 | ET-3 + 30 Liq | Liq/MgAg |
| 22 | HI | HAT-CN | HT-1 | HT-3 | 2-301 | 1-5101 | ET-1 | ET-3 + 30 Liq | Liq/MgAg |
| 23 | HI | HAT-CN | HT-1 | HT-2 | 2-301 | 1-5109 | ET-1 | ET-3 + 30 Liq | Liq/MgAg |
| 24 | HI | HAT-CN | HT-1 | HT-3 | 2-301 | 1-5109 | ET-1 | ET-3 + 30 Liq | Liq/MgAg |

| | | | |
|---|---|---|---|
| Wavelength | Chromaticity | Voltage | |
| (nm) | (x, y) | (V) | External quantum efficiency |

Examples

| | | | | |
|---|---|---|---|---|
| 21 | 454 | (0.142, 0.079) | 3.9 | 5.9 |
| 22 | 454 | (0.143, 0.077) | 3.9 | 6.5 |
| 23 | 454 | (0.140, 0.067) | 3.9 | 6.4 |
| 24 | 454 | (0.140, 0.066) | 4.0 | 6.9 |

In Table 1, “HI” (hole injection layer material) is N⁴,N⁴′-diphenyl-N⁴,N⁴′-bis (9-phenyl-9H-carbazol-3-yl)-[1,1′-biphenyl]-4,4′-diamine, “HAT-CN” (hole injection layer material) is 1,4,5,8,9,12-hexaazatriphenylene hexacarbonitrile, “HT-1” (hole transport layer material) is N-([1,1′-biphenyl]-4-yl)-N-(4-(9-phenyl-9H-carbazol-3-yl) phenyl)-[1,1′-biphenyl]-4-amine, “HT-2” (hole transport layer material) is N,N-bis(4-(dibenzo[b,d]furan-4-yl) phenyl)-[1,1′:4′,1″-terphenyl]-4-amine, “HT-3” (hole transport layer material) is N-([1,1′-biphenyl]-2-yl)-N-(9,9-dimethyl-9H-fluoren-2-yl)-9,9′-spirobi[fluorene]-4-amine, “ET-1” (electron transport layer material) is 4,6,8,10-tetraphenyl[1,4]benzoxabolinino[2,3,4-kl]phenoxabolinine, “ET-2” (electron transport layer material) is 9-(7-(dimesitylboranyl)-9,9-dimethyl-9H-fluoren-2-yl)-3,6-dimethyl-9H-carbazole, “ET-3” (electron transport layer material) is 3,3′-((2-phenylanthracene-9,10-diyl) bis(4,1-phenylene)) bis(4-methylpyridine), and “ET-4” (electron transport layer material) is 4-(3-(4-(10-phenylanthracen-9-yl) naphthalen-1-yl) phenyl) pyridine. The chemical structures thereof are illustrated below together with the chemical structures of “compound (3)” and “Liq”.

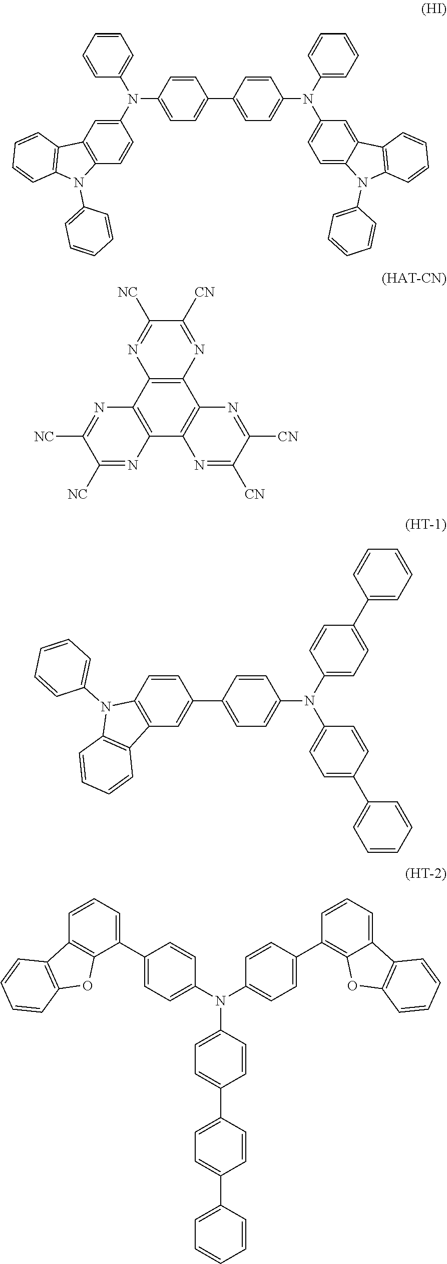 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

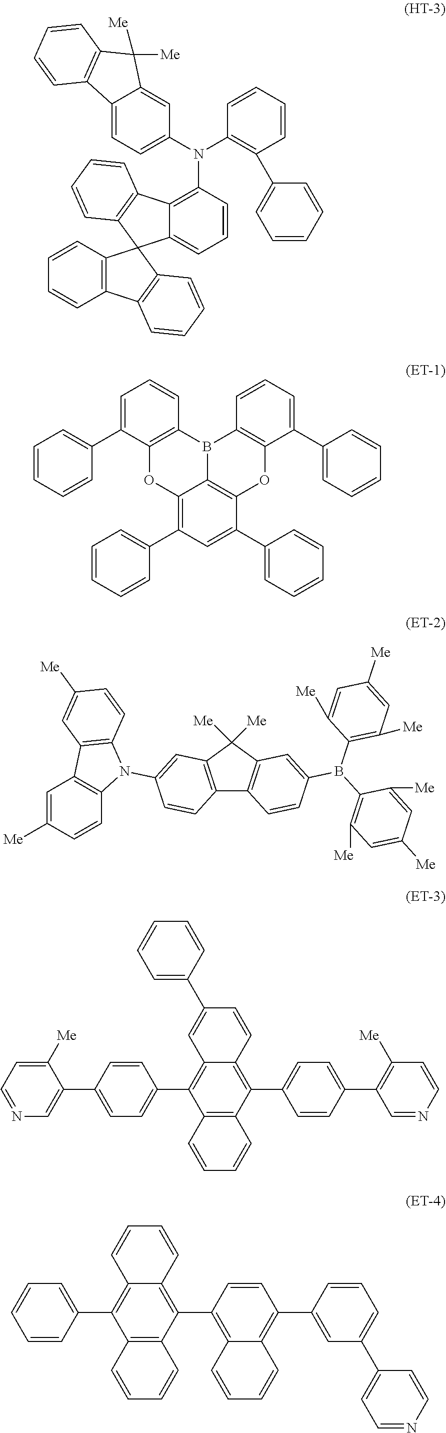 [SMILES: CC1(C)c2ccccc2-c2ccc(N(c3ccccc3-c3ccccc3)c3cccc4c3-c3ccccc3C43c4ccccc4-c4ccccc43)cc21 ; c1ccc(-c2cccc3c2Oc2c(-c4ccccc4)cc(-c4ccccc4)c4c2B3c2cccc(-c3ccccc3)c2O4)cc1 ; Cc1cc(C)c(B(c2ccc3c(c2)C(C)(C)c2cc(-n4c5ccc(C)cc5c5cc(C)ccc54)ccc2-3)c2c(C)cc(C)cc2C)c(C)c1 ; Cc1ccncc1-c1ccc(-c2c3ccccc3c(-c3ccc(-c4cnccc4C)cc3)c3cc(-c4ccccc4)ccc23)cc1 ; c1ccc(-c2c3ccccc3c(-c3ccc(-c4cccc(-c5ccncc5)c4)c4ccccc34)c3ccccc23)cc1]

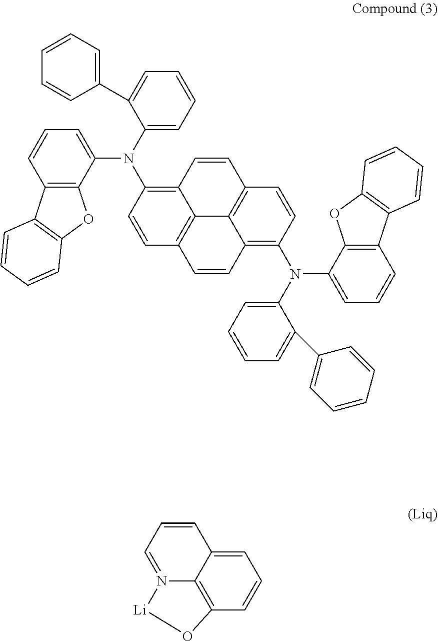

### Example 1

### Element of Host: Compound (2-301), Dopant: Compound (1-2619)

A glass substrate (manufactured by Opto Science, Inc.) having a size of 26 mm×28 mm×0.7 mm, which was obtained by forming a film of ITO having a thickness of 180 nm by sputtering, and polishing the ITO film to 150 nm, was used as a transparent supporting substrate. This transparent supporting substrate was fixed to a substrate holder of a commercially available vapor deposition apparatus (manufactured by Showa Shinku Co., Ltd.), and vapor deposition boats made of molybdenum and containing HI, HAT-CN, HT-1, HT-2, compound (2-301), compound (1-2619), ET-1 and ET-3 respectively, and deposition boats made of aluminum nitride and containing Liq, magnesium, and silver respectively, were mounted in the apparatus.

Layers as described below were formed sequentially on the ITO film of the transparent supporting substrate. The pressure in a vacuum chamber was reduced to 5×10⁻⁴ Pa. Thereafter, HI, HAT-CN, HT-1 and HT-2 were vapor-deposited in this order, to form a hole injection layer 1 (a film thickness of 40 nm), a hole injection layer 2 (a film thickness of 5 nm), a hole transport layer 1 (a film thickness of 15 nm), and a hole transport layer 2 (a film thickness of 10 nm). Subsequently, compound (2-301) and compound (1-2619) were heated simultaneously, and vapor deposition was performed so as to obtain a film thickness of 25 nm to form a light emitting layer. The rate of deposition was regulated such that a weight ratio between compound (2-301) and compound (1-2619) was approximately 98: 2. Subsequently, ET-1 was heated, and vapor deposition was performed so as to obtain a film thickness of 5 nm to form an electron transport layer 1. Subsequently, ET-3 and Liq were heated simultaneously, and vapor deposition was performed so as to obtain a film thickness of 25 nm to form an electron transport layer 2. The rate of deposition was regulated such that the weight ratio between ET-3 and Liq was approximately 50:50. The vapor deposition rate for each layer was 0.01 to 1 nm/sec.

Thereafter, Liq was heated, and vapor deposition was performed at a rate of deposition of 0.01 to 0.1 nm/sec so as to obtain a film thickness of 1 nm, subsequently, magnesium and silver were heated simultaneously, and vapor deposition was performed so as to obtain a film thickness of 100 nm to form a negative electrode, thereby obtaining an organic EL element. At this time, the vapor deposition rate was adjusted in a range between 0.1 nm to 10 nm/sec such that the ratio of the numbers of atoms between magnesium and silver was 10:1.

A direct current voltage was applied using an ITO electrode as a positive electrode and a magnesium/silver electrode as a negative electrode, and characteristics at the time of light emission at 1000 cd/m² were measured. As a result, blue light emission with a wavelength of 462 nm and CIE chromaticity (x, y)=(0.132, 0.085) was obtained. The driving voltage was 4.0 V, and the external quantum efficiency was 6.7%.

### Example 2

### Element of Host: Compound (2-301), Dopant: Compound (1-2619)

An organic EL element was obtained by a method equivalent to that of Example 1, except that material of the hole transport layer 2 was changed to HT-3. Characteristics at the time of light emission at 1000 cd/m² were measured, and blue light emission with a wavelength of 461 nm and CIE chromaticity (x, y)=(0.132, 0.082) was obtained. The driving voltage was 4.1 V, and the external quantum efficiency was 7.2%.

### Example 3

### Element of Host: Compound (2-302), Dopant: Compound (1-2619)

An organic EL element was obtained by a method equivalent to that of Example 1, except that the host material was changed to compound (2-302). Characteristics at the time of light emission at 1000 cd/m² were measured, and blue light emission with a wavelength of 464 nm and CIE chromaticity (x, y)=(0.129, 0.108) was obtained. The driving voltage was 3.9 V, and the external quantum efficiency was 5.8%.

### Example 4

### Element of Host: Compound (2-302), Dopant: Compound (1-2619)

An organic EL element was obtained by a method equivalent to that of Example 1, except that material of the hole transport layer 2 was changed to HT-3 and the host material was changed to compound (2-302). Characteristics at the time of light emission at 1000 cd/m² were measured, and blue light emission with a wavelength of 463 nm and CIE chromaticity (x, y)=(0.129, 0.104) was obtained. The driving voltage was 4.0 V, and the external quantum efficiency was 6.0%.

### Example 5

### Element of Host: Compound (2-302), Dopant: Compound (1-2621)

An organic EL element was obtained by a method equivalent to that of Example 1, except that the host material was changed to compound (2-302), the dopant material was changed to compound (1-2621), material of the electron transport layer 1 was changed to ET-2 and material of the electron transport layer 2 was changed to ET-4 and Liq. Characteristics at the time of light emission at 1000 cd/m² were measured, and blue light emission with a wavelength of 466 nm and CIE chromaticity (x, y)=(0.124, 0.118) was obtained. The driving voltage was 3.9 V, and the external quantum efficiency was 6.0%.

### Example 6

### Element of Host: Compound (2-383), Dopant: Compound (1-2621)

An organic EL element was obtained by a method equivalent to that of Example 1, except that the host material was changed to compound (2-383), the dopant material was changed to compound (1-2621), material of the electron transport layer 1 was changed to ET-2 and material of the electron transport layer 2 was changed to ET-4 and Liq. Characteristics at the time of light emission at 1000 cd/m² were measured, and blue light emission with a wavelength of 467 nm and CIE chromaticity (x, y)=(0.123, 0.121) was obtained. The driving voltage was 3.9 V, and the external quantum efficiency was 5.8%.

### Example 7

### Element of Host: Compound (2-381), Dopant: Compound (1-2621)

An organic EL element was obtained by a method equivalent to that of Example 1, except that the host material was changed to compound (2-381), the dopant material was changed to compound (1-2621), material of the electron transport layer 1 was changed to ET-2 and material of the electron transport layer 2 was changed to ET-4 and Liq. Characteristics at the time of light emission at 1000 cd/m² were measured, and blue light emission with a wavelength of 466 nm and CIE chromaticity (x, y)=(0.126, 0.108) was obtained. The driving voltage was 4.1 V, and the external quantum efficiency was 5.9%.

### Example 8

### Element of Host: Compound (2-381), Dopant: Compound (1-2619)

An organic EL element was obtained by a method equivalent to that of Example 1, except that material of the hole transport layer 2 was changed to HT-3 and the host material was changed to compound (2-381). Characteristics at the time of light emission at 1000 cd/m² were measured, and blue light emission with a wavelength of 463 nm and CIE chromaticity (x, y)=(0.130, 0.095) was obtained. The driving voltage was 4.3 V, and the external quantum efficiency was 6.6%.

### Example 9

### Element of Host: Compound (2-382), Dopant: Compound (1-2619)

An organic EL element was obtained by a method equivalent to that of Example 1, except that material of the hole transport layer 2 was changed to HT-3 and the host material was changed to compound (2-382). Characteristics at the time of light emission at 1000 cd/m² were measured, and blue light emission with a wavelength of 462 nm and CIE chromaticity (x, y)=(0.132, 0.100) was obtained. The driving voltage was 4.1 V, and the external quantum efficiency was 6.0%.

### Example 10

### Element of Host: Compound (2-101), Dopant: Compound (1-2619)

An organic EL element was obtained by a method equivalent to that of Example 1, except that material of the hole transport layer 2 was changed to HT-3 and the host material was changed to compound (2-101). Characteristics at the time of light emission at 1000 cd/m² were measured, and blue light emission with a wavelength of 462 nm and CIE chromaticity (x, y)=(0.131, 0.087) was obtained. The driving voltage was 4.4 V, and the external quantum efficiency was 6.4%.

### Example 11

### Element of Host: Compound (2-202), Dopant: Compound (1-2619)

An organic EL element was obtained by a method equivalent to that of Example 1, except that material of the hole transport layer 2 was changed to HT-3 and the host material was changed to compound (2-202). Characteristics at the time of light emission at 1000 cd/m² were measured, and blue light emission with a wavelength of 464 nm and CIE chromaticity (x, y)=(0.127, 0.101) was obtained. The driving voltage was 4.3 V, and the external quantum efficiency was 7.1%.

### Example 12

### Element of Host: Compound (2-303), Dopant: Compound (1-2619)

An organic EL element was obtained by a method equivalent to that of Example 1, except that material of the hole transport layer 2 was changed to HT-3 and the host material was changed to compound (2-303). Characteristics at the time of light emission at 1000 cd/m² were measured, and blue light emission with a wavelength of 462 nm and CIE chromaticity (x, y)=(0.130, 0.093) was obtained. The driving voltage was 4.4 V, and the external quantum efficiency was 6.6%.

### Example 13

### Element of Host: Compound (2-401), Dopant: Compound (1-2619)

An organic EL element was obtained by a method equivalent to that of Example 1, except that the host material was changed to compound (2-401). Characteristics at the time of light emission at 1000 cd/m² were measured, and blue light emission with a wavelength of 463 nm and CIE chromaticity (x, y)=(0.132, 0.087) was obtained. The driving voltage was 3.9 V, and the external quantum efficiency was 6.2%.

### Example 14

### Element of Host: Compound (2-401), Dopant: Compound (1-2619)

An organic EL element was obtained by a method equivalent to that of Example 1, except that material of the hole transport layer 2 was changed to HT-3 and the host material was changed to compound (2-401). Characteristics at the time of light emission at 1000 cd/m² were measured, and blue light emission with a wavelength of 463 nm and CIE chromaticity (x, y)=(0.130, 0.092) was obtained. The driving voltage was 3.8 V, and the external quantum efficiency was 6.5%.

### Example 15

### Element of Host: Compound (2-427), Dopant: Compound (1-2619)

An organic EL element was obtained by a method equivalent to that of Example 1, except that the host material was changed to compound (2-427). Characteristics at the time of light emission at 1000 cd/m² were measured, and blue light emission with a wavelength of 462 nm and CIE chromaticity (x, y)=(0.134, 0.079) was obtained. The driving voltage was 3.8 V, and the external quantum efficiency was 6.8%.

### Example 16

### Element of Host: Compound (2-419), Dopant: Compound (1-2619)

An organic EL element was obtained by a method equivalent to that of Example 1, except that the host material was changed to compound (2-419). Characteristics at the time of light emission at 1000 cd/m² were measured, and blue light emission with a wavelength of 463 nm and CIE chromaticity (x, y)=(0.130, 0.101) was obtained. The driving voltage was 4.0 V, and the external quantum efficiency was 6.4%.

### Example 17

### Element of Host: Compound (2-411), Dopant: Compound (1-2619)

An organic EL element was obtained by a method equivalent to that of Example 1, except that the host material was changed to compound (2-411). Characteristics at the time of light emission at 1000 cd/m² were measured, and blue light emission with a wavelength of 462 nm and CIE chromaticity (x, y)=(0.133, 0.087) was obtained. The driving voltage was 4.0 V, and the external quantum efficiency was 6.2%.

### Example 18

### Element of Host: Compound (2-660), Dopant: Compound (1-2619)

An organic EL element was obtained by a method equivalent to that of Example 1, except that material of the hole transport layer 2 was changed to HT-3 and the host material was changed to compound (2-660). Characteristics at the time of light emission at 1000 cd/m² were measured, and blue light emission with a wavelength of 462 nm and CIE chromaticity (x, y)=(0.132, 0.082) was obtained. The driving voltage was 4.3 V, and the external quantum efficiency was 7.9%.

### Example 19

### Element of Host: Compound (2-643), Dopant: Compound (1-2619)

An organic EL element was obtained by a method equivalent to that of Example 1, except that material of the hole transport layer 2 was changed to HT-3 and the host material was changed to compound (2-643). Characteristics at the time of light emission at 1000 cd/m² were measured, and blue light emission with a wavelength of 463 nm and CIE chromaticity (x, y)=(0.130, 0.095) was obtained. The driving voltage was 3.4 V, and the external quantum efficiency was 5.9%.

### Example 20

### Element of Host: Compound (2-662), Dopant: Compound (1-2619)

An organic EL element was obtained by a method equivalent to that of Example 1, except that the host material was changed to compound (2-662). Characteristics at the time of light emission at 1000 cd/m² were measured, and blue light emission with a wavelength of 461 nm and CIE chromaticity (x, y)=(0.136, 0.078) was obtained. The driving voltage was 4.4 V, and the external quantum efficiency was 5.4%.

### Comparative Example 1

### Element of Host: Compound (2-301), Dopant: Compound (3)

An organic EL element was obtained by a method equivalent to that of Example 1, except that the dopant material was changed to compound (3). Characteristics at the time of light emission at 1000 cd/m² were measured, and blue light emission with a wavelength of 460 nm and CIE chromaticity (x, y)=(0.134, 0.120) was obtained. The driving voltage was 4.1 V, and the external quantum efficiency was 4.8%.

### Comparative Example 2

### Element of Host: Compound (2-301), Dopant: Compound (3)

An organic EL element was obtained by a method equivalent to that of Example 1, except that material of the hole transport layer 2 was changed to HT-3 and the dopant material was changed to compound (3).

Characteristics at the time of light emission at 1000 cd/m² were measured, and blue light emission with a wavelength of 460 nm and CIE chromaticity (x, y)=(0.134, 0.120) was obtained. The driving voltage was 4.1 V, and the external quantum efficiency was 4.8%.

### Comparative Example 3

### Element of Host: Compound (2-302), Dopant: Compound (3)

An organic EL element was obtained by a method equivalent to that of Example 1, except that the host material was changed to compound (2-302) and the dopant material was changed to compound (3). Characteristics at the time of light emission at 1000 cd/m² were measured, and blue light emission with a wavelength of 462 nm and CIE chromaticity (x, y)=(0.133, 0.152) was obtained. The driving voltage was 3.9 V, and the external quantum efficiency was 4.5%.

### Comparative Example 4

### Element of Host: Compound (2-302), Dopant: Compound (3)

An organic EL element was obtained by a method equivalent to that of Example 1, except that material of the hole transport layer 2 was changed to HT-3, the host material was changed to compound (2-302) and the dopant material was changed to compound (3). Characteristics at the time of light emission at 1000 cd/m² were measured, and blue light emission with a wavelength of 462 nm and CIE chromaticity (x, y)=(0.134, 0.145) was obtained. The driving voltage was 4.0 V, and the external quantum efficiency was 4.8%.

### Comparative Example 5

### Element of Host: Compound (2-381), Dopant: Compound (3)

An organic EL element was obtained by a method equivalent to that of Example 1, except that material of the hole transport layer 2 was changed to HT-3, the host material was changed to compound (2-381) and the dopant material was changed to compound (3). Characteristics at the time of light emission at 1000 cd/m² were measured, and blue light emission with a wavelength of 460 nm and CIE chromaticity (x, y)=(0.133, 0.123) was obtained. The driving voltage was 4.5 V, and the external quantum efficiency was 4.1%.

### Comparative Example 6

### Element of Host: Compound (2-401), Dopant: Compound (3)

An organic EL element was obtained by a method equivalent to that of Example 1, except that material of the hole transport layer 1 was changed to HT-3, the host material was changed to compound (2-401) and the dopant material was changed to compound (3). Characteristics at the time of light emission at 1000 cd/m² were measured, and blue light emission with a wavelength of 460 nm and CIE chromaticity (x, y)=(0.135, 0.116) was obtained. The driving voltage was 4.1 V, and the external quantum efficiency was 4.1%.

### Example 21

### Element of Host: Compound (2-301), Dopant: Compound (1-5101)

A glass substrate (manufactured by Opto Science, Inc.) having a size of 26 mm×28 mm×0.7 mm, which was obtained by forming a film of ITO having a thickness of 180 nm by sputtering, and polishing the ITO film to 150 nm, was used as a transparent supporting substrate. This transparent supporting substrate was fixed to a substrate holder of a commercially available vapor deposition apparatus (manufactured by Showa Shinku Co., Ltd.), and vapor deposition boats made of molybdenum and containing HI, HAT-CN, HT-1, HT-2, compound (2-301), compound (1-5101), ET-1 and ET-3 respectively, and deposition boats made of aluminum nitride and containing Liq, magnesium, and silver respectively, were mounted in the apparatus.

Layers as described below were formed sequentially on the ITO film of the transparent supporting substrate. The pressure in a vacuum chamber was reduced to 5×10⁻⁴ Pa. Thereafter, HI, HAT-CN, HT-1 and HT-2 were vapor-deposited in this order, to form a hole injection layer 1 (a film thickness of 40 nm), a hole injection layer 2 (a film thickness of 5 nm), a hole transport layer 1 (a film thickness of 15 nm), and a hole transport layer 2 (a film thickness of 10 nm). Subsequently, compound (2-301) and compound (1-5101) were heated simultaneously, and vapor deposition was performed so as to obtain a film thickness of 25 nm to form a light emitting layer. The rate of deposition was regulated such that a weight ratio between compound (2-301) and compound (1-5101) was approximately 98:2. Subsequently, ET-1 was heated, and vapor deposition was performed so as to obtain a film thickness of 5 nm to form an electron transport layer 1. Subsequently, ET-3 and Liq were heated simultaneously, and vapor deposition was performed so as to obtain a film thickness of 25 nm to form an electron transport layer 2. The rate of deposition was regulated such that the weight ratio between ET-3 and Liq was approximately 50:50. The vapor deposition rate for each layer was 0.01 to 1 nm/sec.

Thereafter, Liq was heated, and vapor deposition was performed at a rate of deposition of 0.01 to 0.1 nm/sec so as to obtain a film thickness of 1 nm, subsequently, magnesium and silver were heated simultaneously, and vapor deposition was performed so as to obtain a film thickness of 100 nm to form a negative electrode, thereby obtaining an organic EL element. At this time, the vapor deposition rate was adjusted in a range between 0.1 nm to 10 nm/sec such that the ratio of the numbers of atoms between magnesium and silver was 10:1.

A direct current voltage was applied using an ITO electrode as a positive electrode and a magnesium/silver electrode as a negative electrode, and characteristics at the time of light emission at 1000 cd/m² were measured. As a result, blue light emission with a wavelength of 454 nm and CIE chromaticity (x, y)=(0.142, 0.079) was obtained. The driving voltage was 3.9 V, and the external quantum efficiency was 5.9%.

### Example 22

### Element of Host: Compound (2-301), Dopant: Compound (1-5101)

An organic EL element was obtained by a method equivalent to that of Example 21, except that material of the hole transport layer 2 was changed to HT-3. Characteristics at the time of light emission at 1000 cd/m² were measured, and blue light emission with a wavelength of 454 nm and CIE chromaticity (x, y)=(0.143, 0.077) was obtained. The driving voltage was 3.9 V, and the external quantum efficiency was 6.5%.

### Example 23

### Element of Host: Compound (2-301), Dopant: Compound (1-5109)

An organic EL element was obtained by a method equivalent to that of Example 21, except that the dopant material was changed to compound (1-5109). Characteristics at the time of light emission at 1000 cd/m² were measured, and blue light emission with a wavelength of 454 nm and CIE chromaticity (x, y)=(0.140, 0.067) was obtained. The driving voltage was 3.9 V, and the external quantum efficiency was 6.4%.

### Example 24

### Element of Host: Compound (2-301), Dopant: Compound (1-5109)

An organic EL element was obtained by a method equivalent to that of Example 21, except that material of the hole transport layer 2 was changed to HT-3 and the dopant material was changed to compound (1-5109). Characteristics at the time of light emission at 1000 cd/m² were measured, and blue light emission with a wavelength of 454 nm and CIE chromaticity (x, y)=(0.140, 0.066) was obtained. The driving voltage was 4.0 V, and the external quantum efficiency was 6.9%.

### INDUSTRIAL APPLICABILITY

According to a preferable embodiment of the present invention, it is possible to provide a compound represented by formula (1) and a compound represented by formula (2), capable of obtaining optimum light emitting characteristics in combination with the compound represented by formula (1). By manufacturing an organic EL element using a material for a light emitting layer obtained by combining these compounds, it is possible to provide an organic EL element that is excellent in at least one of chromaticity, driving voltage, quantum efficiency, and lifetime of element.

### REFERENCE SIGNS LIST

100 Organic electroluminescent element
101 Substrate
102 Positive electrode
103 Hole injection layer
104 Hole transport layer
105 Light emitting layer
106 Electron transport layer
107 Electron injection layer
108 Negative electrode

1. An organic electroluminescent element comprising a pair of electrodes composed of a positive electrode and a negative electrode and a light emitting layer disposed between the pair of electrodes, in which the light emitting layer comprises at least one selected from the group consisting of a compound represented by the following general formula (1) and a multimer having a plurality of structures represented by the following general formula (1), and a compound represented by the following general formula (2). 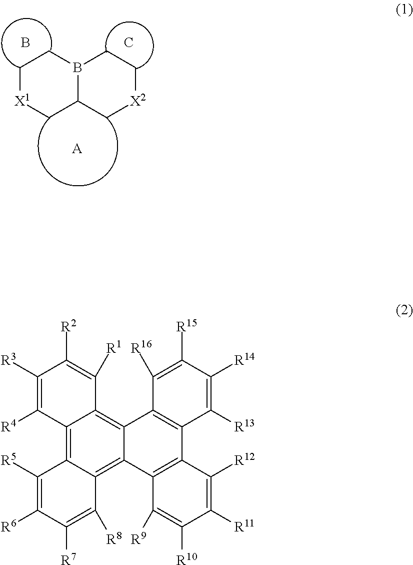
(In the above formula (1),
ring A, ring B and ring C each independently represent an aryl ring or a heteroaryl ring, while at least one hydrogen atom in these rings may be substituted,
X¹ and X² each independently represent O or N—R, R of the N—R is an optionally substituted aryl, an optionally substituted heteroaryl or an optionally substituted alkyl, R of the N—R may be bonded to the ring A, ring B, and/or ring C with a linking group or a single bond, and
at least one hydrogen atom in a compound or a structure represented by formula (1) may be substituted by a halogen atom, a cyano or a deuterium atom)
(In the above formula (2),
R¹ to R¹⁶ each independently represent a hydrogen atom, an aryl, a heteroaryl (the heteroaryl may be bonded to the dibenzochrysene skeleton in the above formula (2) via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl,
adjacent groups among R¹ to R¹⁶ may be bonded to each other to form a fused ring, and at least one hydrogen atom in the formed ring may be substituted by an aryl, a heteroaryl (the heteroaryl may be bonded to the formed ring via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, and
at least one hydrogen atom in the compound represented by formula (2) may be substituted by a halogen atom, a cyano, or a deuterium atom.)

2. The organic electroluminescent element described in claim 1, in which
in the above formula (2),
R¹, R⁴, R⁵, R⁸, R⁹, R¹², R¹³, and R¹⁶ each represent a hydrogen atom,
R², R³, R⁶, R⁷, R¹⁰, R¹¹, R¹⁴, and R¹⁵ each independently represent a hydrogen atom, an aryl, a heteroaryl (the heteroaryl may be bonded to the dibenzochrysene skeleton in the above formula (2) via a linking group), a diarylamino, a diheteroarylamino, an arylheteroarylamino, an alkyl, an alkenyl, an alkoxy, or an aryloxy, while at least one hydrogen atom in these may be substituted by an aryl, a heteroaryl, or an alkyl, and
at least one hydrogen atom in the compound represented by the above formula (2) may be substituted by a halogen atom, a cyano, or a deuterium atom.

3. The organic electroluminescent element described in claim 1, in which
in the above formula (2),
R¹, R⁴, R⁵, R⁸, R⁹, R¹², R¹³, and R¹⁶ each represent a hydrogen atom,
R², R³, R⁶, R⁷, R¹⁰, R¹¹, R¹⁴, and R¹⁵ each independently represent a hydrogen atom, an aryl having 6 to 30 carbon atoms, a heteroaryl having 2 to 30 carbon atoms (the heteroaryl may be bonded to the dibenzochrysene skeleton in the above formula (2) via a linking group), a diarylamino having 8 to 30 carbon atoms, a diheteroarylamino having 4 to 30 carbon atoms, an arylheteroarylamino having 4 to 30 carbon atoms, an alkyl having 1 to 30 carbon atoms, an alkenyl having 1 to 30 carbon atoms, an alkoxy having 1 to 30 carbon atoms, or an aryloxy having 1 to 30 carbon atoms, while at least one hydrogen atom in these may be substituted by an aryl having 6 to 14 carbon atoms, a heteroaryl having 2 to 20 carbon atoms, or an alkyl having 1 to 12 carbon atoms, and
at least one hydrogen atom in the compound represented by the above formula (2) may be substituted by a halogen atom, a cyano, or a deuterium atom.

4. The organic electroluminescent element described in claim 1, in which
in the above formula (2),
R¹, R⁴, R⁵, R⁸, R⁹, R¹², R¹³, and R¹⁶ each represent a hydrogen atom,
R², R³, R⁶, R⁷, R¹⁰, R¹¹, R¹⁴, and R¹⁵ each represent a hydrogen atom, a phenyl, a biphenylyl, a naphthyl, an anthracenyl, a phenanthrenyl, a monovalent group having a structure of the following formula (2-Ar1), (2-Ar2), (2-Ar3), (2-Ar4), or (2-Ar5) (the monovalent group having the structure may be bonded to the dibenzochrysene skeleton in the above formula (2) via a phenylene, a biphenylene, a naphthylene, an anthracenylene, a methylene, an ethylene, —OCH₂CH₂—, —CH₂CH₂O—, or —OCH₂CH₂O—), a methyl, an ethyl, a propyl, or a butyl, while at least one hydrogen atom in these may be substituted by a phenyl, a biphenylyl, a naphthyl, an anthracenyl, a phenanthrenyl, a monovalent group having a structure of the following formula (2-Ar1), (2-Ar2), (2-Ar3), (2-Ar4), or (2-Ar5), a methyl, an ethyl, a propyl, or a butyl, and
at least one hydrogen atom in the compound represented by the above formula (2) may be substituted by a halogen atom, a cyano, or a deuterium atom. 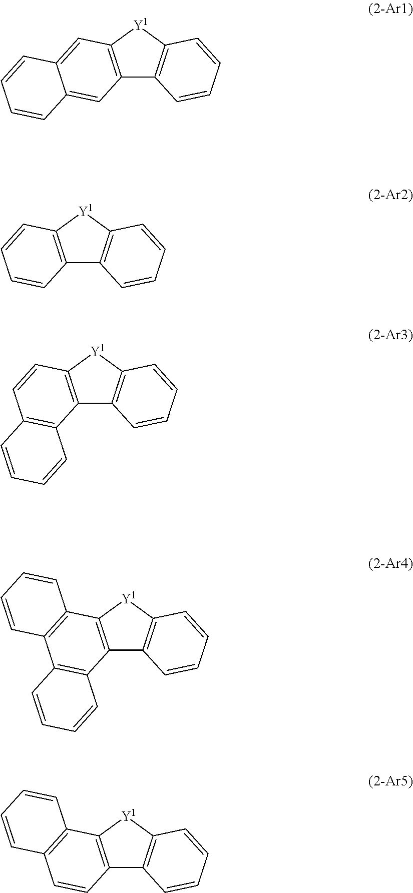
(In the above formulas (2-Ar1) to (2-Ar5), Y¹'s each independently represent O, S, or N—R, and R represents a phenyl, a biphenylyl, a naphthyl, an anthracenyl, or a hydrogen atom,
at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl, and
at least one hydrogen atom in the structures represented by the above formulas (2-Ar1) to (2-Ar5) may be bonded to any one of R¹ to R¹⁶ in the above formula (2) to form a single bond.)

5. The organic electroluminescent element described in claim 1, in which
in the above formula (2),
R¹, R², R⁴, R⁵, R⁷, R⁸, R⁹, R¹⁰, R¹², R¹³, R¹⁵, and R¹⁶ each represent a hydrogen atom,
at least one of R³, R⁶, R¹¹, and R¹⁴ represents a monovalent group having a structure of the following formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,
a group other than the at least one represents a hydrogen atom, a phenyl, a biphenylyl, a naphthyl, an anthracenyl, a methyl, an ethyl, a propyl, or a butyl, while at least one hydrogen atom in these may be substituted by a phenyl, a biphenylyl, a naphthyl, an anthracenyl, a methyl, an ethyl, a propyl, or a butyl, and
at least one hydrogen atom in the compound represented by the above formula (2) may be substituted by a halogen atom, a cyano, or a deuterium atom. 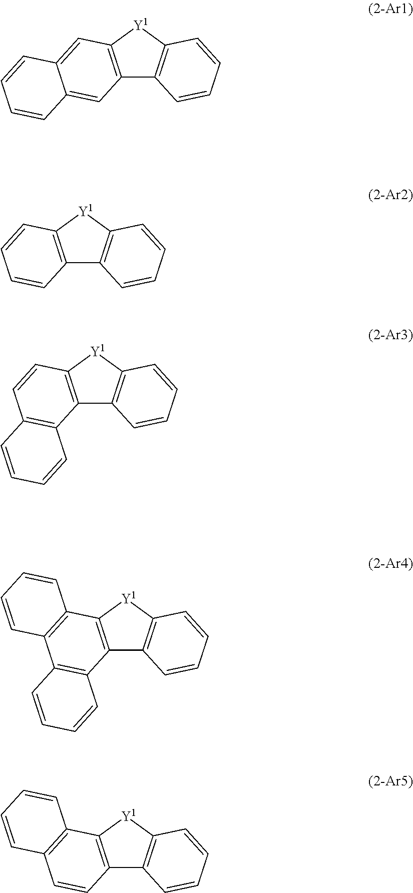
(In the formulas (2-Ar1) to (2-Ar5), Y¹'s each independently represent O, S, or N—R, and R represents a phenyl, a biphenylyl, a naphthyl, an anthracenyl, or a hydrogen atom, and
at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.)

6. The organic electroluminescent element described in claim 5, in which
in the above formula (2),
R¹, R², R⁴, R⁵, R⁷, R⁸, R⁹, R¹⁰, R¹², R¹³, R¹⁵, and R¹⁶ each represent a hydrogen atom,
at least one of R³, R⁶, R¹¹, and R¹⁴ represents a monovalent group having a structure of the above formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,
a group other than the at least one represents a hydrogen atom, a phenyl, a biphenylyl, a naphthyl, an anthracenyl, a methyl, an ethyl, a propyl, or a butyl,
at least one hydrogen atom in the compound represented by the above formula (2) may be substituted by a halogen atom, a cyano, or a deuterium atom,
in the above formulas (2-Ar1) to (2-Ar5), Y¹'s each independently represent O, S, or N—R, and R represents a phenyl, a biphenylyl, a naphthyl, an anthracenyl, or a hydrogen atom, and
at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.

7. The organic electroluminescent element described in claim 1, wherein the compound represented by the above formula (2) is a compound represented by any of the following structural formulas.

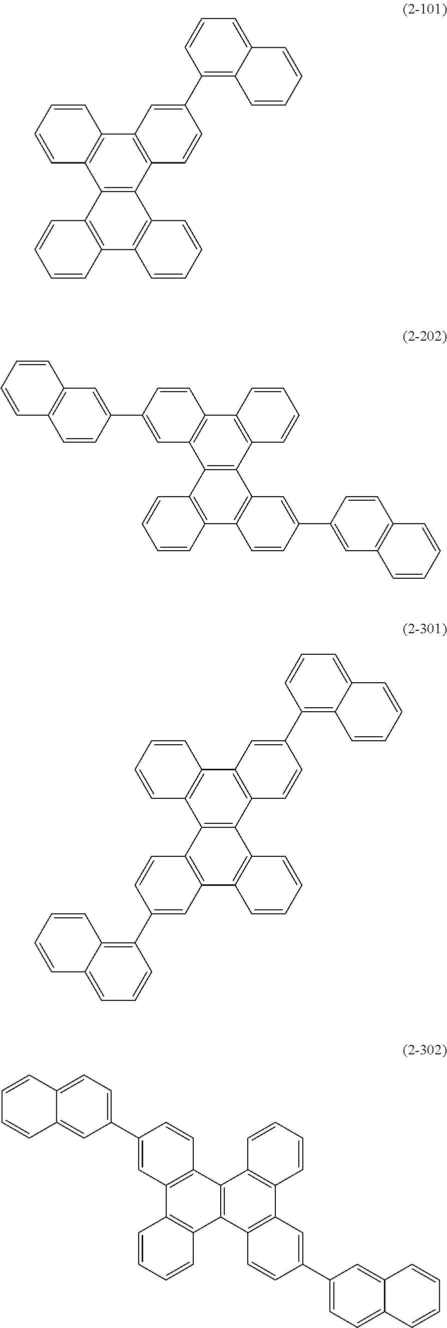

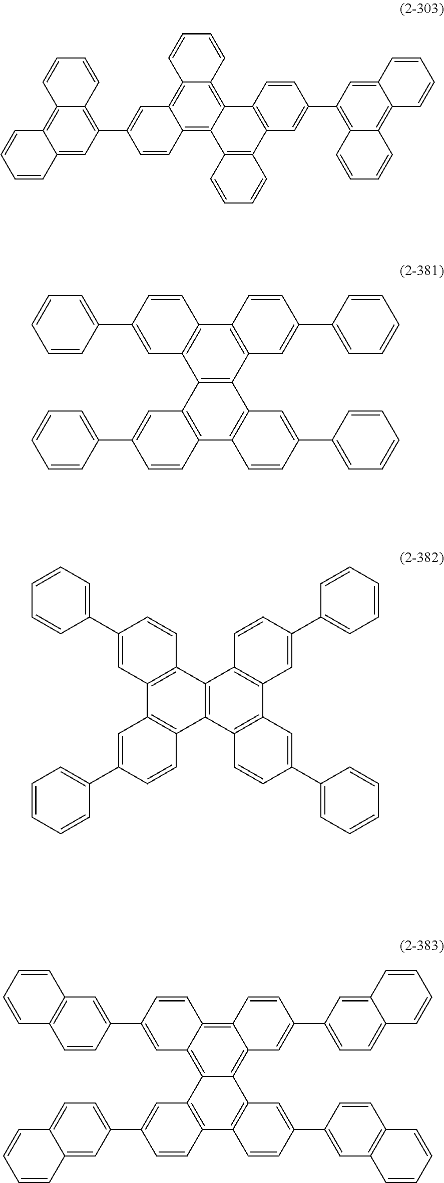

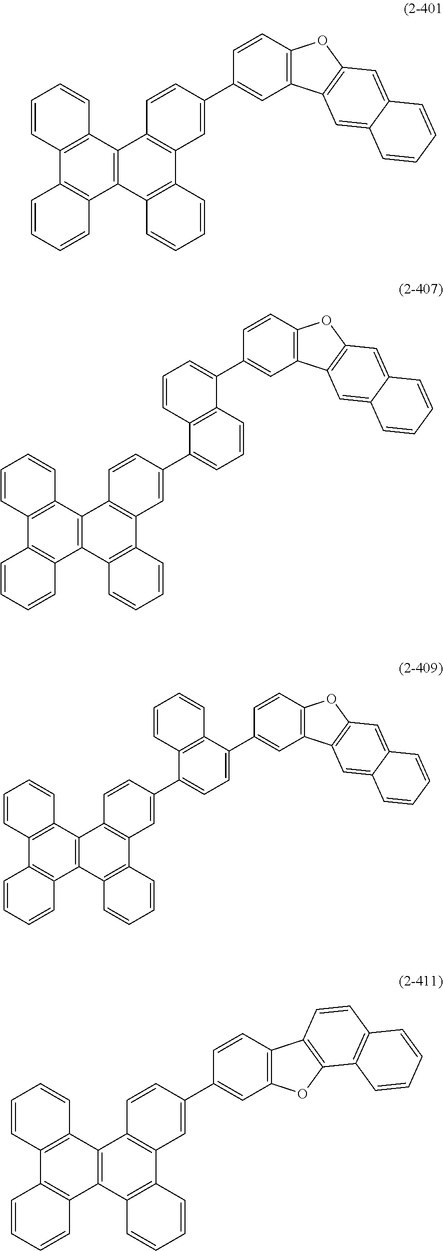 [SMILES: c1ccc2cc3c(cc2c1)oc1ccc(-c2ccc4c(c2)c2ccccc2c2c5ccccc5c5ccccc5c42)cc13 ; c1ccc2cc3c(cc2c1)oc1ccc(-c2cccc4c(-c5ccc6c(c5)c5ccccc5c5c7ccccc7c7ccccc7c65)cccc24)cc13 ; c1ccc2cc3c(cc2c1)oc1ccc(-c2ccc(-c4ccc5c(c4)c4ccccc4c4c6ccccc6c6ccccc6c54)c4ccccc24)cc13 ; c1ccc2c(c1)ccc1c3ccc(-c4ccc5c(c4)c4ccccc4c4c6ccccc6c6ccccc6c54)cc3oc21]

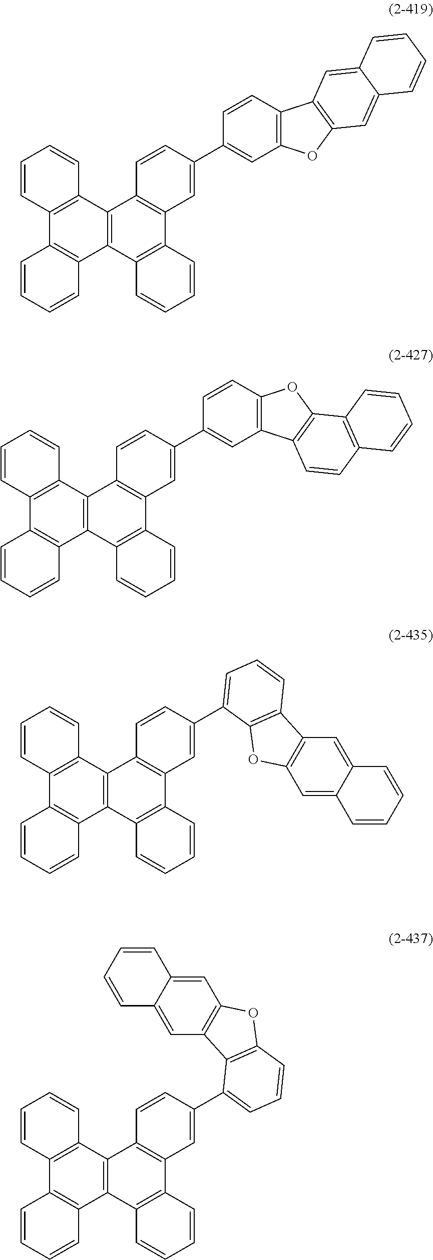 [SMILES: c1ccc2cc3c(cc2c1)oc1cc(-c2ccc4c(c2)c2ccccc2c2c5ccccc5c5ccccc5c42)ccc13 ; c1ccc2c(c1)ccc1c3cc(-c4ccc5c(c4)c4ccccc4c4c6ccccc6c6ccccc6c54)ccc3oc21 ; c1ccc2cc3c(cc2c1)oc1c(-c2ccc4c(c2)c2ccccc2c2c5ccccc5c5ccccc5c42)cccc13 ; c1ccc2cc3c(cc2c1)oc1cccc(-c2ccc4c(c2)c2ccccc2c2c5ccccc5c5ccccc5c42)c13]

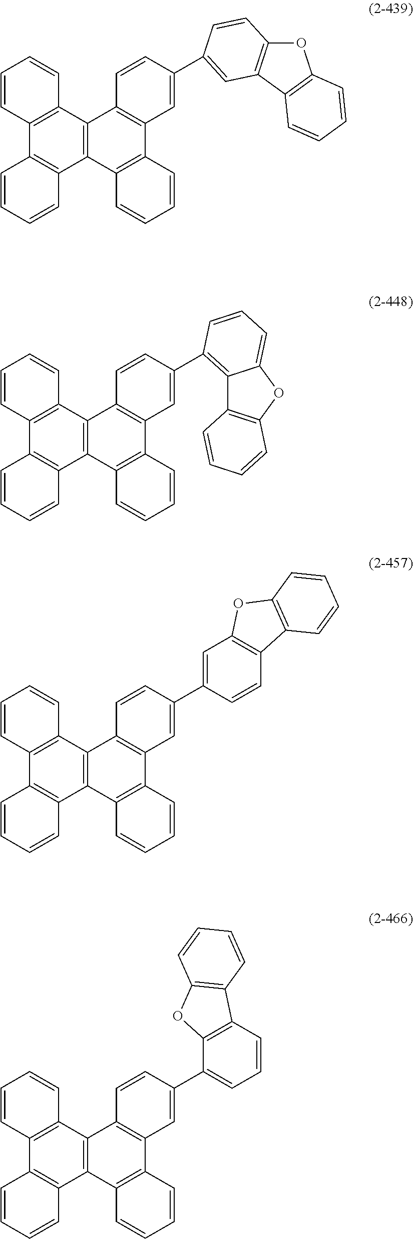 [SMILES: c1ccc2c(c1)oc1ccc(-c3ccc4c(c3)c3ccccc3c3c5ccccc5c5ccccc5c43)cc12 ; c1ccc2c(c1)oc1cccc(-c3ccc4c(c3)c3ccccc3c3c5ccccc5c5ccccc5c43)c12 ; c1ccc2c(c1)oc1cc(-c3ccc4c(c3)c3ccccc3c3c5ccccc5c5ccccc5c43)ccc12 ; c1ccc2c(c1)oc1c(-c3ccc4c(c3)c3ccccc3c3c5ccccc5c5ccccc5c43)cccc12]

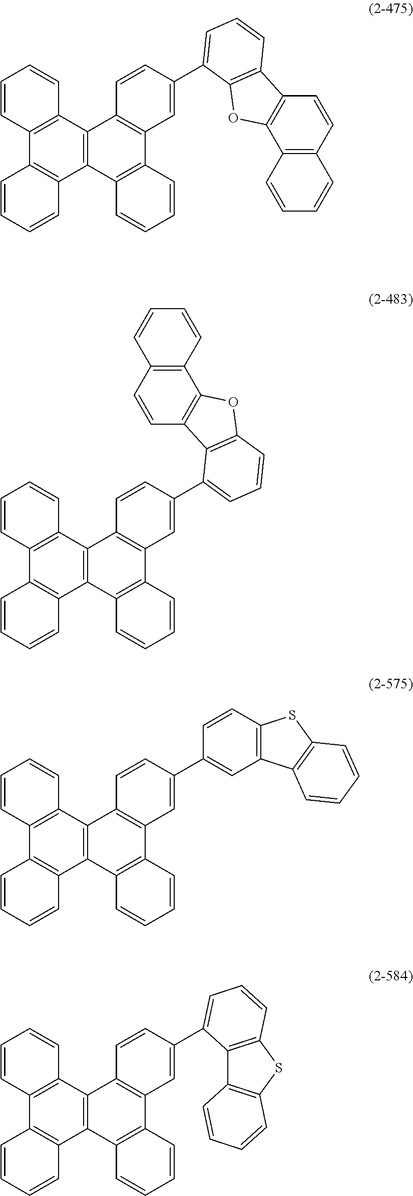 [SMILES: c1ccc2c(c1)ccc1c3cccc(-c4ccc5c(c4)c4ccccc4c4c6ccccc6c6ccccc6c54)c3oc21 ; c1ccc2c(c1)ccc1c2oc2cccc(-c3ccc4c(c3)c3ccccc3c3c5ccccc5c5ccccc5c43)c21 ; c1ccc2c(c1)sc1ccc(-c3ccc4c(c3)c3ccccc3c3c5ccccc5c5ccccc5c43)cc12 ; c1ccc2c(c1)sc1cccc(-c3ccc4c(c3)c3ccccc3c3c5ccccc5c5ccccc5c43)c12]

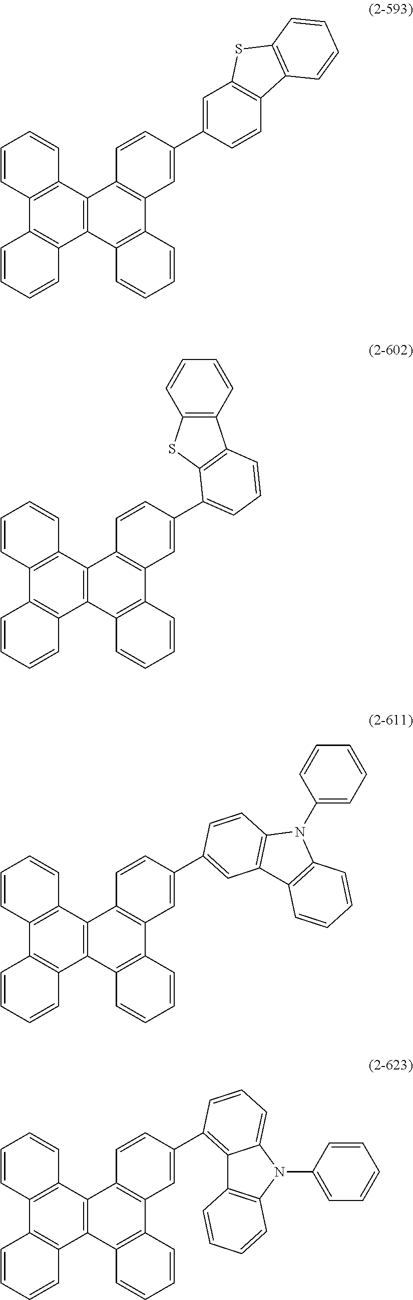 [SMILES: c1ccc2c(c1)sc1cc(-c3ccc4c(c3)c3ccccc3c3c5ccccc5c5ccccc5c43)ccc12 ; c1ccc2c(c1)sc1c(-c3ccc4c(c3)c3ccccc3c3c5ccccc5c5ccccc5c43)cccc12 ; c1ccc(-n2c3ccccc3c3cc(-c4ccc5c(c4)c4ccccc4c4c6ccccc6c6ccccc6c54)ccc32)cc1 ; c1ccc(-n2c3ccccc3c3c(-c4ccc5c(c4)c4ccccc4c4c6ccccc6c6ccccc6c54)cccc32)cc1]

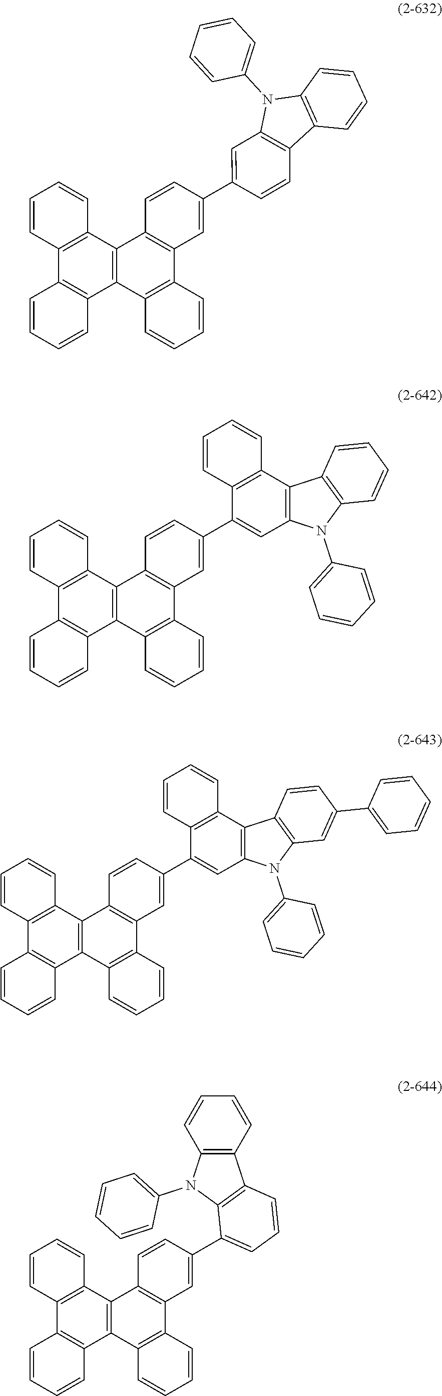 [SMILES: c1ccc(-n2c3ccccc3c3ccc(-c4ccc5c(c4)c4ccccc4c4c6ccccc6c6ccccc6c54)cc32)cc1 ; c1ccc(-n2c3ccccc3c3c4ccccc4c(-c4ccc5c(c4)c4ccccc4c4c6ccccc6c6ccccc6c54)cc32)cc1 ; c1ccc(-c2ccc3c4c5ccccc5c(-c5ccc6c(c5)c5ccccc5c5c7ccccc7c7ccccc7c65)cc4n(-c4ccccc4)c3c2)cc1 ; c1ccc(-n2c3ccccc3c3cccc(-c4ccc5c(c4)c4ccccc4c4c6ccccc6c6ccccc6c54)c32)cc1]

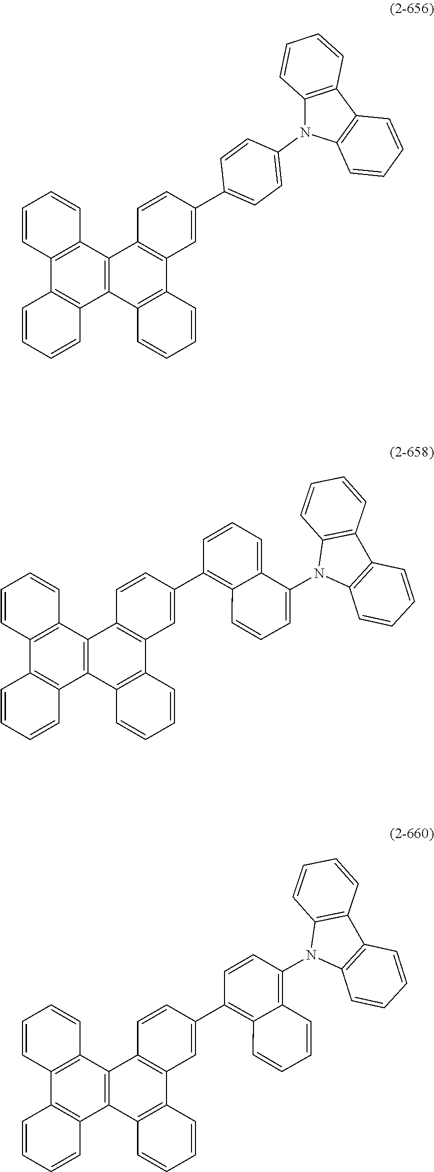 [SMILES: c1ccc2c(c1)c1ccccc1c1c3ccc(-c4ccc(-n5c6ccccc6c6ccccc65)cc4)cc3c3ccccc3c21 ; c1cc(-n2c3ccccc3c3ccccc32)c2cccc(-c3ccc4c(c3)c3ccccc3c3c5ccccc5c5ccccc5c43)c2c1 ; c1ccc2c(-n3c4ccccc4c4ccccc43)ccc(-c3ccc4c(c3)c3ccccc3c3c5ccccc5c5ccccc5c43)c2c1]

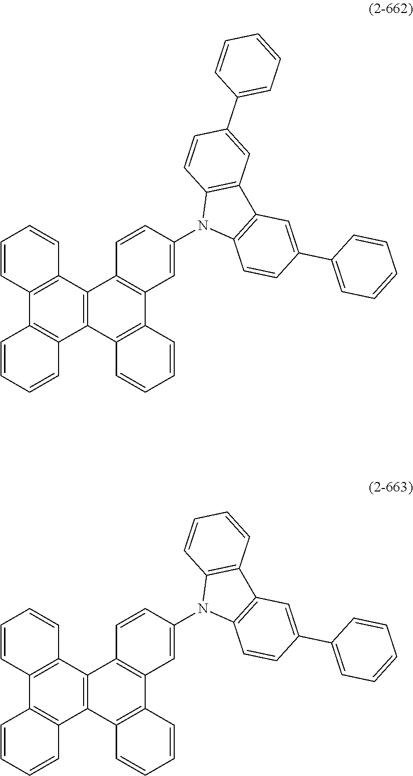 [SMILES: c1ccc(-c2ccc3c(c2)c2cc(-c4ccccc4)ccc2n3-c2ccc3c(c2)c2ccccc2c2c4ccccc4c4ccccc4c32)cc1 ; c1ccc(-c2ccc3c(c2)c2ccccc2n3-c2ccc3c(c2)c2ccccc2c2c4ccccc4c4ccccc4c32)cc1]

8. The organic electroluminescent element described in claim 1, in which
in the above formula (1),
the ring A, ring B, and ring C each independently represent an aryl ring or a heteroaryl ring, while at least one hydrogen atom in these rings may be substituted by a substituted or unsubstituted aryl, a substituted or unsubstituted heteroaryl, a substituted or unsubstituted diarylamino, a substituted or unsubstituted diheteroarylamino, a substituted or unsubstituted arylheteroarylamino, a substituted or unsubstituted alkyl, a substituted or unsubstituted alkoxy, or a substituted or unsubstituted aryloxy, each of these rings has a 5-membered or 6-membered ring sharing a bond with a fused bicyclic structure at the center of the above formula constructed by B, X¹, and X²,
X¹ and X² each independently represent O or N—R, R of the N—R each independently represents an aryl which may be substituted by an alkyl, a heteroaryl which may be substituted by an alkyl or alkyl, R of the N—R may be bonded to the ring A, ring B, and/or ring C with —O—, —S—, —C(—R)₂— or a single bond, R of the —C(—R)₂— represents a hydrogen atom or an alkyl,
at least one hydrogen atom in a compound or structure represented by formula (1) may be substituted by a halogen atom, a cyano or a deuterium atom, and
in a case of a multimer, the multimer is a dimer or a trimer having two or three structures represented by formula (1).

9. The organic electroluminescent element described in claim 1, wherein the compound represented by the above general formula (1) is a compound represented by the following general formula (1′). 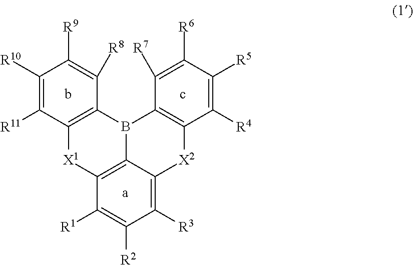
(In the above formula (1′),
R¹ to R¹¹ each independently represent a hydrogen atom, an aryl, a heteroaryl, a diarylamino, a diheteroarylamino, an arylheteroarylamino, an alkyl, an alkoxy, or an aryloxy, while at least one hydrogen atom in these may be substituted by an aryl, a heteroaryl, or an alkyl, adjacent groups among R¹ to R¹¹ may be bonded to each other to form an aryl ring or a heteroaryl ring together with ring a, ring b, or ring c, at least one hydrogen atom in the ring thus formed may be substituted by an aryl, a heteroaryl, a diarylamino, a diheteroarylamino, an arylheteroarylamino, an alkyl, an alkoxy, or an aryloxy, at least one hydrogen atom in these may be substituted by an aryl, a heteroaryl or an alkyl,
X¹ and X² each independently represent N—R, R of the N—R represents an aryl having 6 to 12 carbon atoms, a heteroaryl having 2 to 15 carbon atoms, or an alkyl having 1 to 6 carbon atoms, R of the N—R may be bonded to the ring a, ring b and/or ring c with —O—, —S—, —C(—R)₂—, or a single bond, R of the —C(—R)₂— represents an alkyl having 1 to 6 carbon atoms, and
at least one hydrogen atom in a compound represented by formula (1′) may be substituted by a halogen atom or a deuterium atom.)

10. The organic electroluminescent element described in claim 9, in which
in the above formula (1′),
R¹ to R¹¹ each independently represent a hydrogen atom, an aryl having 6 to 30 carbon atoms, a heteroaryl having 2 to 30 carbon atoms or a diarylamino (the aryl is an aryl having 6 to 12 carbon atoms), adjacent groups among R¹ to R¹¹ may be bonded to each other to form an aryl ring having 9 to 16 carbon atoms or a heteroaryl ring having 6 to 15 carbon atoms together with the ring a, ring b, or ring c, at least one hydrogen atom in the ring thus formed may be substituted by an aryl having 6 to 10 carbon atoms,
X¹ and X² each independently represent N—R, R of the N—R is an aryl having 6 to 10 carbon atoms, and
at least one hydrogen atom in a compound represented by formula (1′) may be substituted by a halogen atom or a deuterium atom.

11. The organic electroluminescent element described in claim 1, wherein the compound represented by the above formula (1) is a compound represented by any of the following structural formulas.

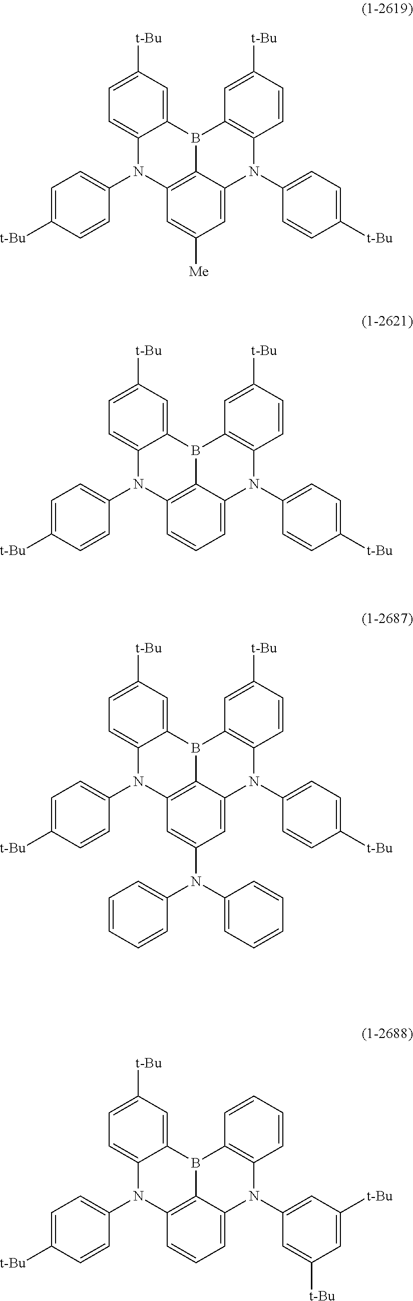 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; CC(C)(C)c1ccc(N2c3ccc(C(C)(C)C)cc3B3c4ccccc4N(c4cc(C(C)(C)C)cc(C(C)(C)C)c4)c4cccc2c43)cc1]

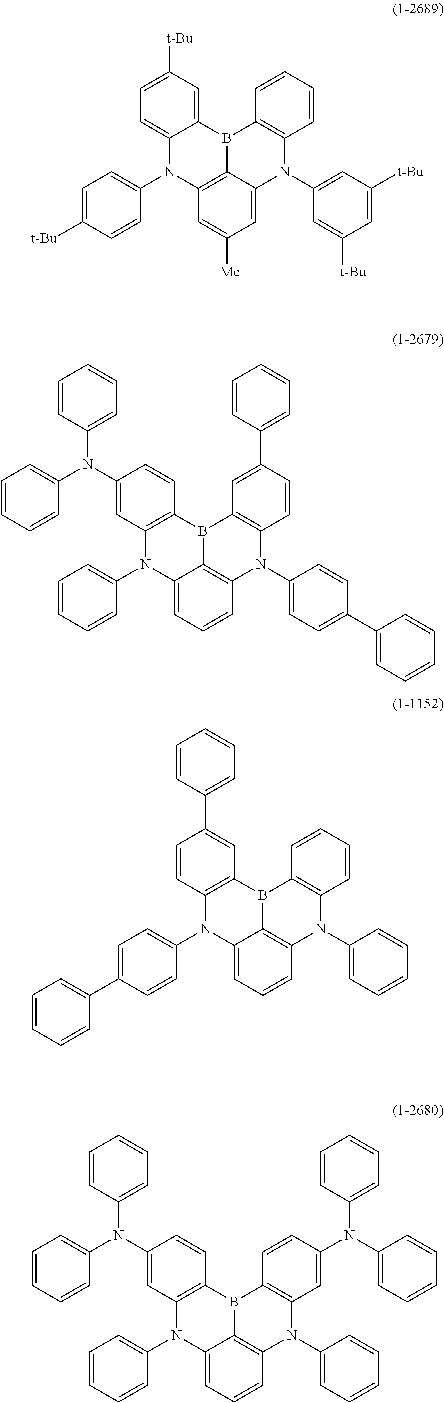 [SMILES: Cc1cc2c3c(c1)N(c1ccc(C(C)(C)C)cc1)c1ccc(C(C)(C)C)cc1B3c1ccccc1N2c1cc(C(C)(C)C)cc(C(C)(C)C)c1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

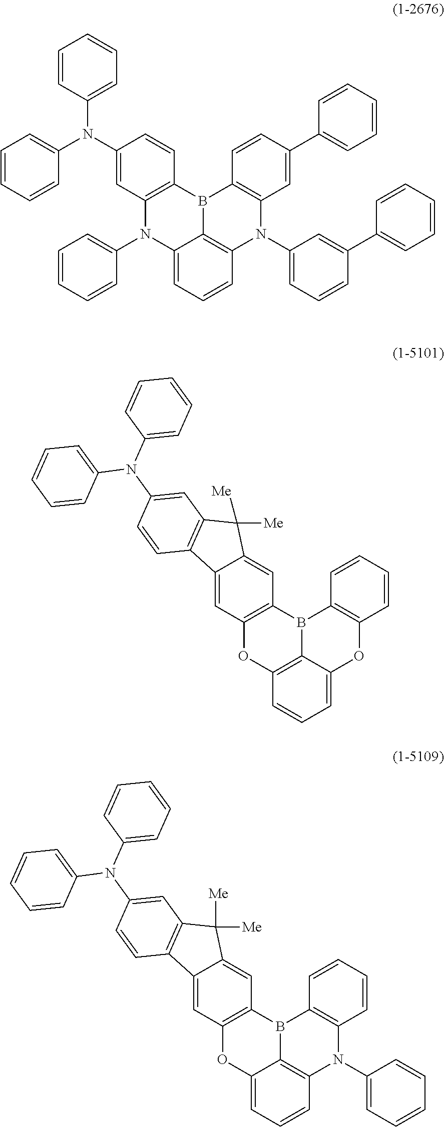 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; CC1(C)c2cc(N(c3ccccc3)c3ccccc3)ccc2-c2cc3c(cc21)B1c2ccccc2Oc2cccc(c21)O3 ; CC1(C)c2cc(N(c3ccccc3)c3ccccc3)ccc2-c2cc3c(cc21)B1c2ccccc2N(c2ccccc2)c2cccc(c21)O3]

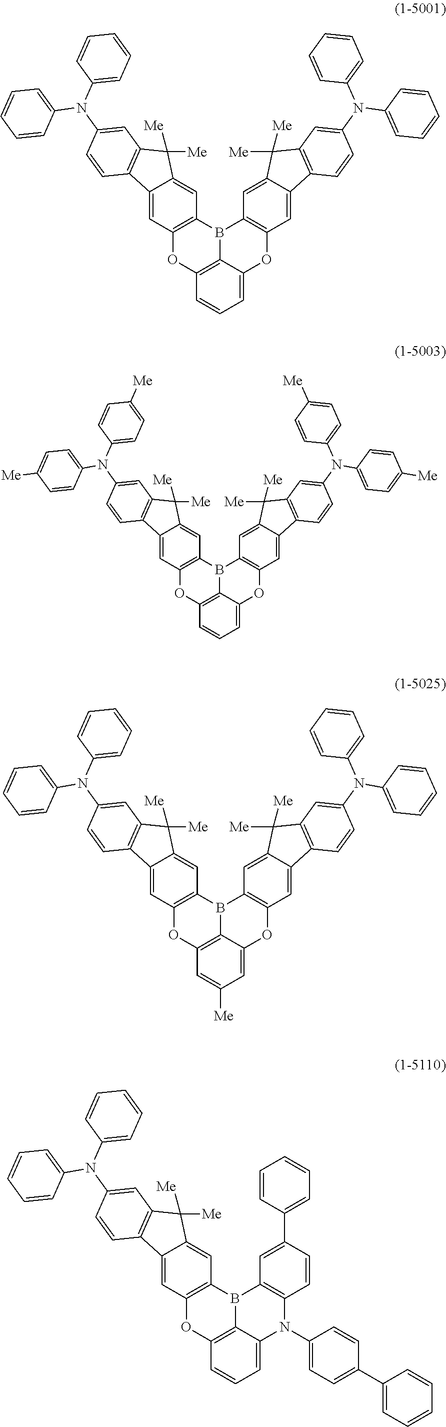 [SMILES: CC1(C)c2cc(N(c3ccccc3)c3ccccc3)ccc2-c2cc3c(cc21)B1c2cc4c(cc2Oc2cccc(c21)O3)-c1ccc(N(c2ccccc2)c2ccccc2)cc1C4(C)C ; Cc1ccc(N(c2ccc(C)cc2)c2ccc3c(c2)C(C)(C)c2cc4c(cc2-3)Oc2cccc3c2B4c2cc4c(cc2O3)-c2ccc(N(c3ccc(C)cc3)c3ccc(C)cc3)cc2C4(C)C)cc1 ; Cc1cc2c3c(c1)Oc1cc4c(cc1B3c1cc3c(cc1O2)-c1ccc(N(c2ccccc2)c2ccccc2)cc1C3(C)C)C(C)(C)c1cc(N(c2ccccc2)c2ccccc2)ccc1-4 ; CC1(C)c2cc(N(c3ccccc3)c3ccccc3)ccc2-c2cc3c(cc21)B1c2cc(-c4ccccc4)ccc2N(c2ccc(-c4ccccc4)cc2)c2cccc(c21)O3]

12. The organic electroluminescent element described in claim 1, further comprising an electron transport layer and/or an electron injection layer disposed between the negative electrode and the light emitting layer, in which at least one of the electron transport layer and the electron injection layer comprises at least one selected from the group consisting of a borane derivative, a pyridine derivative, a fluoranthene derivative, a BO-based derivative, an anthracene derivative, a benzofluorene derivative, a phosphine oxide derivative, a pyrimidine derivative, a carbazole derivative, a triazine derivative, a benzimidazole derivative, a phenanthroline derivative, and a quinolinol-based metal complex.

13. The organic electroluminescent element described in claim 12, in which the electron transport layer and/or electron injection layer further comprise/comprises at least one selected from the group consisting of an alkali metal, an alkaline earth metal, a rare earth metal, an oxide of an alkali metal, a halide of an alkali metal, an oxide of an alkaline earth metal, a halide of an alkaline earth metal, an oxide of a rare earth metal, a halide of a rare earth metal, an organic complex of an alkali metal, an organic complex of an alkaline earth metal, and an organic complex of a rare earth metal.

14. A display apparatus comprising the organic electroluminescent element described in claim 1.

15. A lighting apparatus comprising the organic electroluminescent element described in claim 1.

16. A compound represented by the following formula (2). 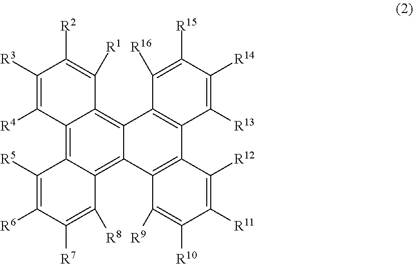
(In the above formula (2),
R¹, R², R⁴, R⁵, R⁷, R⁸, R⁹, R¹⁰, R¹², R¹³, R¹⁵, and R¹⁶ each represent a hydrogen atom,
at least one of R³, R⁶, R¹¹, and R¹⁴ represents a monovalent group having a structure of the following formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,
a group other than the at least one represents a hydrogen atom, a phenyl, a biphenylyl, a naphthyl, an anthracenyl, a methyl, an ethyl, a propyl, or a butyl, while at least one hydrogen atom in these may be substituted by a phenyl, a biphenylyl, a naphthyl, an anthracenyl, a methyl, an ethyl, a propyl, or a butyl, and
at least one hydrogen atom in the compound represented by the above formula (2) may be substituted by a halogen atom, a cyano, or a deuterium atom. 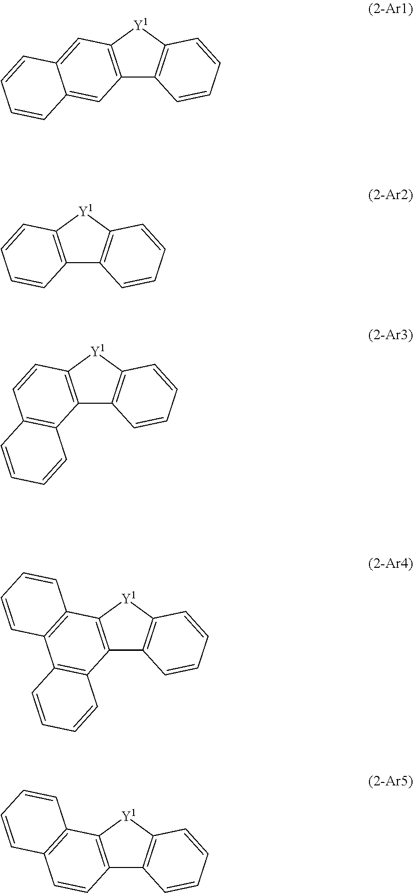
(In the formulas (2-Ar1) to (2-Ar5), Y¹'s each independently represent O, S, or N—R, and R represents a phenyl, a biphenylyl, a naphthyl, an anthracenyl, or a hydrogen atom, and
at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.)

17. The compound described in claim 16, in which
in the above formula (2),
R¹, R², R⁴, R⁵, R⁷, R⁸, R⁹, R¹⁰, R¹², R¹³, R¹⁵, and R¹⁶ each represent a hydrogen atom,
at least one of R³, R⁶, R¹¹, and R¹⁴ represents a monovalent group having a structure of the above formula (2-Ar1), (2-Ar2), (2-Ar3), (2-Ar4), or (2-Ar5) via a single bond, a phenylene, a biphenylene, a naphthylene, an anthracenylene, a methylene, an ethylene, —OCH₂CH₂—, —CH₂CH₂O—, or —OCH₂CH₂O—,
a group other than the at least one represents a hydrogen atom, a phenyl, a biphenylyl, a naphthyl, an anthracenyl, a methyl, an ethyl, a propyl, or a butyl,
at least one hydrogen atom in the compound represented by the above formula (2) may be substituted by a halogen atom, a cyano, or a deuterium atom,
in the above formulas (2-Ar1) to (2-Ar5), Y¹'s each independently represent O, S, or N—R, and R represents a phenyl, a biphenylyl, a naphthyl, an anthracenyl, or a hydrogen atom, and
at least one hydrogen atom in the structures of the above formulas (2-Ar1) to (2-Ar5) may be substituted by a phenyl, a biphenylyl, a naphthyl, an anthracenyl, a phenanthrenyl, a methyl, an ethyl, a propyl, or a butyl.

18. A compound represented by any of the following structural formulas. 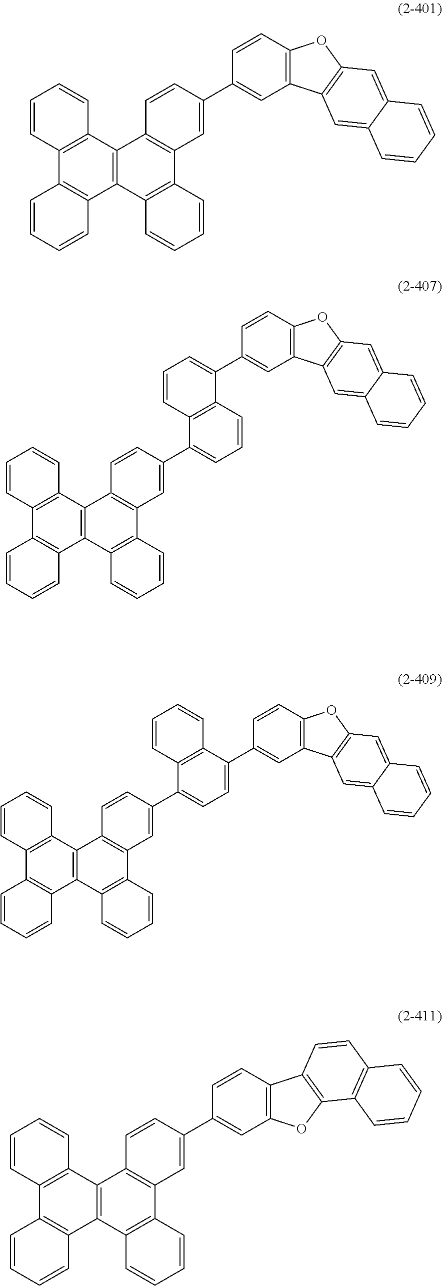 [SMILES: c1ccc2cc3c(cc2c1)oc1ccc(-c2ccc4c(c2)c2ccccc2c2c5ccccc5c5ccccc5c42)cc13 ; c1ccc2cc3c(cc2c1)oc1ccc(-c2cccc4c(-c5ccc6c(c5)c5ccccc5c5c7ccccc7c7ccccc7c65)cccc24)cc13 ; c1ccc2cc3c(cc2c1)oc1ccc(-c2ccc(-c4ccc5c(c4)c4ccccc4c4c6ccccc6c6ccccc6c54)c4ccccc24)cc13 ; c1ccc2c(c1)ccc1c3ccc(-c4ccc5c(c4)c4ccccc4c4c6ccccc6c6ccccc6c54)cc3oc21] 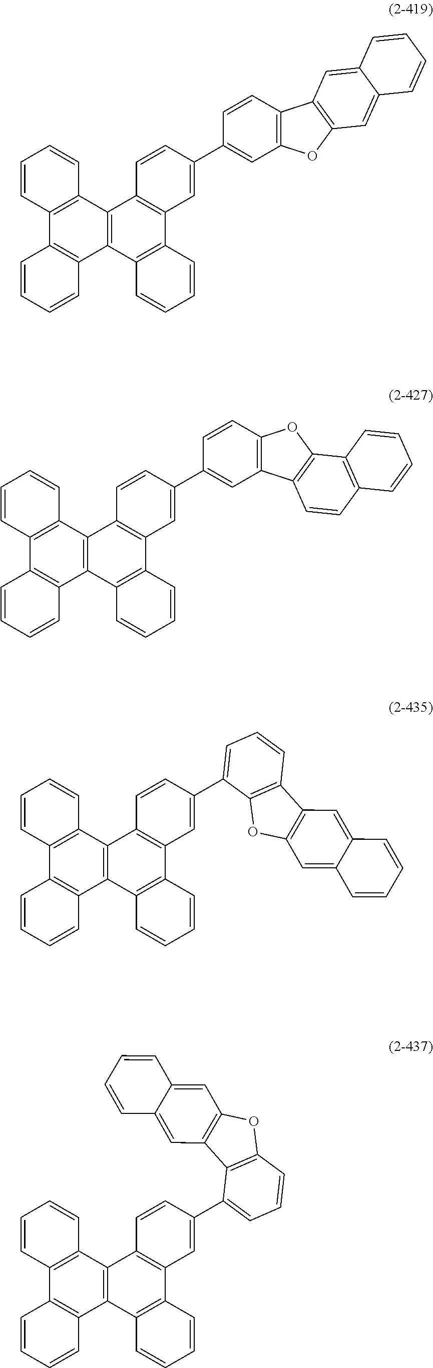 [SMILES: c1ccc2cc3c(cc2c1)oc1cc(-c2ccc4c(c2)c2ccccc2c2c5ccccc5c5ccccc5c42)ccc13 ; c1ccc2c(c1)ccc1c3cc(-c4ccc5c(c4)c4ccccc4c4c6ccccc6c6ccccc6c54)ccc3oc21 ; c1ccc2cc3c(cc2c1)oc1c(-c2ccc4c(c2)c2ccccc2c2c5ccccc5c5ccccc5c42)cccc13 ; c1ccc2cc3c(cc2c1)oc1cccc(-c2ccc4c(c2)c2ccccc2c2c5ccccc5c5ccccc5c42)c13] 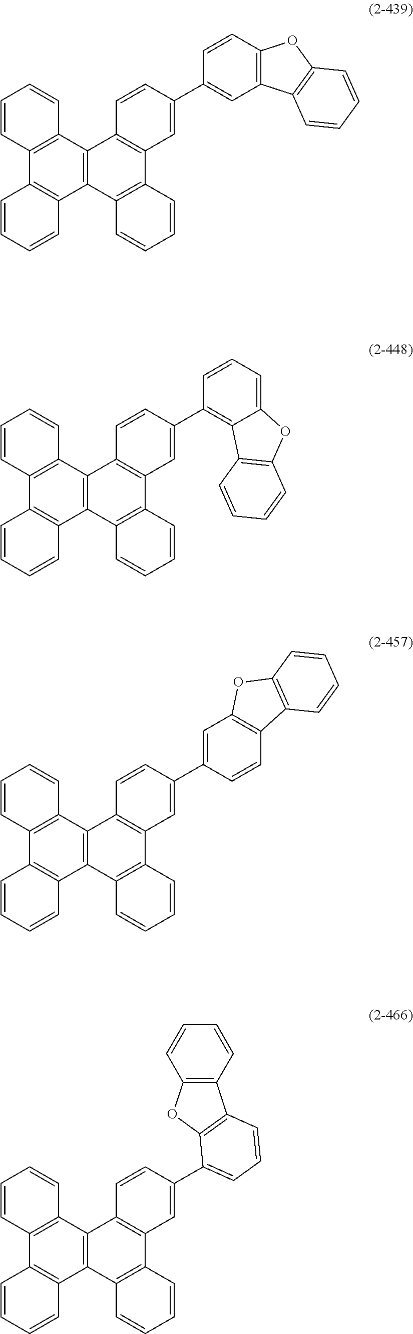 [SMILES: c1ccc2c(c1)oc1ccc(-c3ccc4c(c3)c3ccccc3c3c5ccccc5c5ccccc5c43)cc12 ; c1ccc2c(c1)oc1cccc(-c3ccc4c(c3)c3ccccc3c3c5ccccc5c5ccccc5c43)c12 ; c1ccc2c(c1)oc1cc(-c3ccc4c(c3)c3ccccc3c3c5ccccc5c5ccccc5c43)ccc12 ; c1ccc2c(c1)oc1c(-c3ccc4c(c3)c3ccccc3c3c5ccccc5c5ccccc5c43)cccc12] 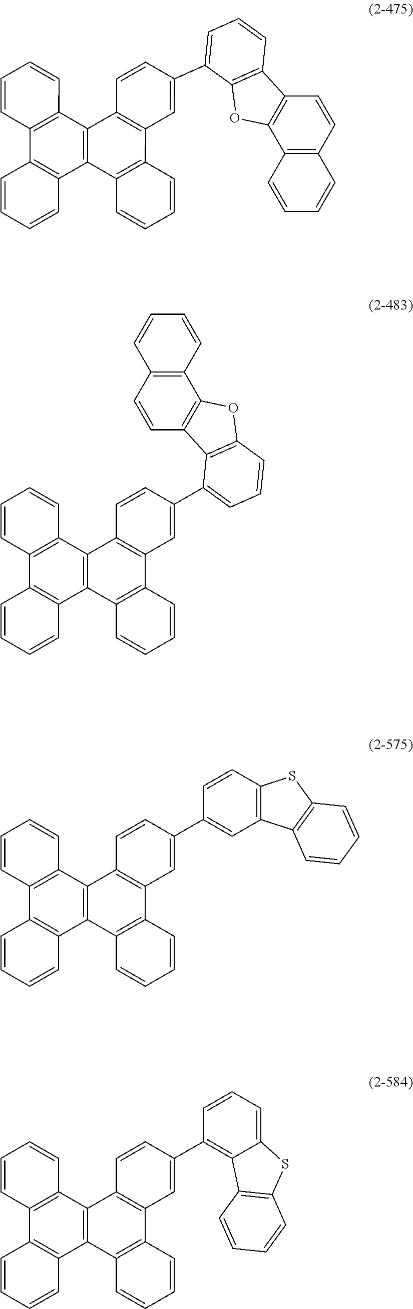 [SMILES: c1ccc2c(c1)ccc1c3cccc(-c4ccc5c(c4)c4ccccc4c4c6ccccc6c6ccccc6c54)c3oc21 ; c1ccc2c(c1)ccc1c2oc2cccc(-c3ccc4c(c3)c3ccccc3c3c5ccccc5c5ccccc5c43)c21 ; c1ccc2c(c1)sc1ccc(-c3ccc4c(c3)c3ccccc3c3c5ccccc5c5ccccc5c43)cc12 ; c1ccc2c(c1)sc1cccc(-c3ccc4c(c3)c3ccccc3c3c5ccccc5c5ccccc5c43)c12] 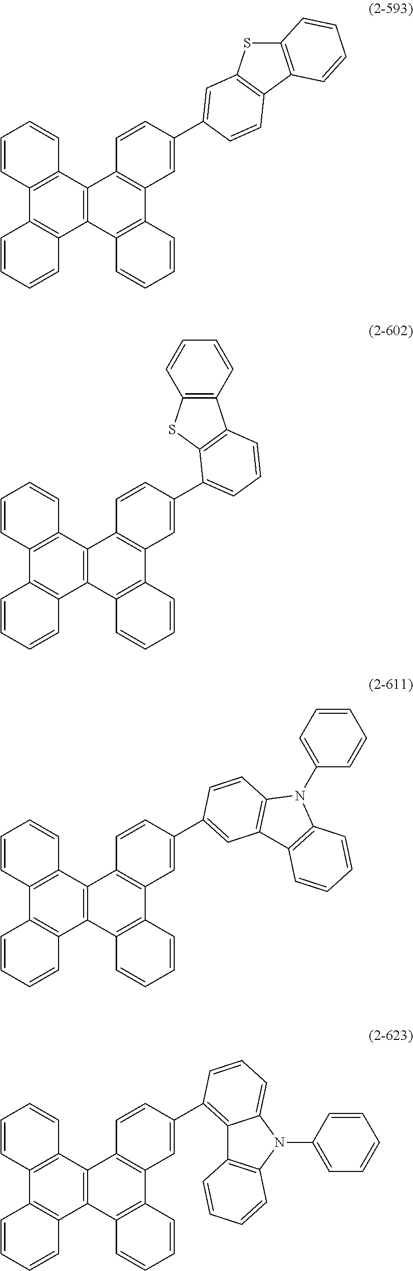 [SMILES: c1ccc2c(c1)sc1cc(-c3ccc4c(c3)c3ccccc3c3c5ccccc5c5ccccc5c43)ccc12 ; c1ccc2c(c1)sc1c(-c3ccc4c(c3)c3ccccc3c3c5ccccc5c5ccccc5c43)cccc12 ; c1ccc(-n2c3ccccc3c3cc(-c4ccc5c(c4)c4ccccc4c4c6ccccc6c6ccccc6c54)ccc32)cc1 ; c1ccc(-n2c3ccccc3c3c(-c4ccc5c(c4)c4ccccc4c4c6ccccc6c6ccccc6c54)cccc32)cc1] 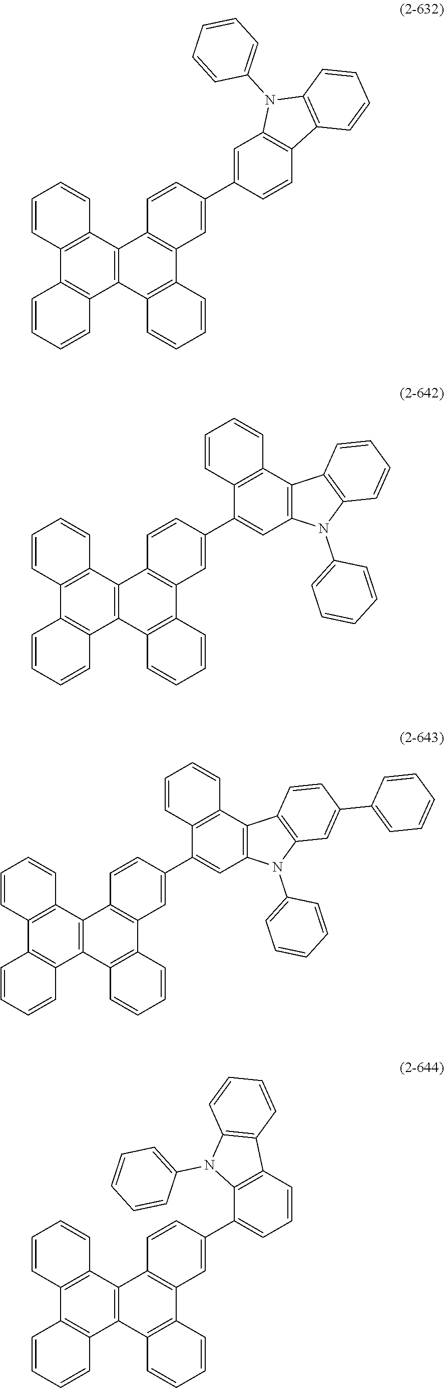 [SMILES: c1ccc(-n2c3ccccc3c3ccc(-c4ccc5c(c4)c4ccccc4c4c6ccccc6c6ccccc6c54)cc32)cc1 ; c1ccc(-n2c3ccccc3c3c4ccccc4c(-c4ccc5c(c4)c4ccccc4c4c6ccccc6c6ccccc6c54)cc32)cc1 ; c1ccc(-c2ccc3c4c5ccccc5c(-c5ccc6c(c5)c5ccccc5c5c7ccccc7c7ccccc7c65)cc4n(-c4ccccc4)c3c2)cc1 ; c1ccc(-n2c3ccccc3c3cccc(-c4ccc5c(c4)c4ccccc4c4c6ccccc6c6ccccc6c54)c32)cc1] 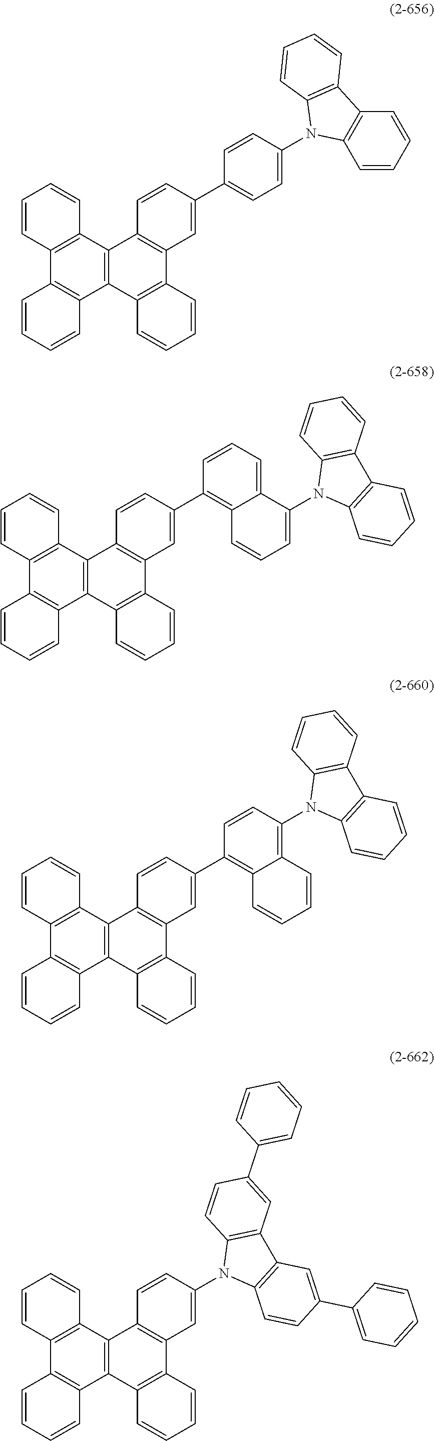 [SMILES: c1ccc2c(c1)c1ccccc1c1c3ccc(-c4ccc(-n5c6ccccc6c6ccccc65)cc4)cc3c3ccccc3c21 ; c1cc(-n2c3ccccc3c3ccccc32)c2cccc(-c3ccc4c(c3)c3ccccc3c3c5ccccc5c5ccccc5c43)c2c1 ; c1ccc2c(-n3c4ccccc4c4ccccc43)ccc(-c3ccc4c(c3)c3ccccc3c3c5ccccc5c5ccccc5c43)c2c1 ; c1ccc(-c2ccc3c(c2)c2cc(-c4ccccc4)ccc2n3-c2ccc3c(c2)c2ccccc2c2c4ccccc4c4ccccc4c32)cc1] 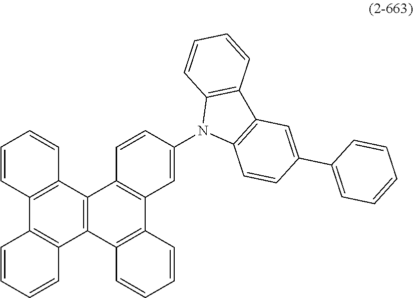 [SMILES: c1ccc(-c2ccc3c(c2)c2ccccc2n3-c2ccc3c(c2)c2ccccc2c2c4ccccc4c4ccccc4c32)cc1]
